SEQUENCE LISTING (7 sequences)

SEQ ID NO: 1 - protein, 637 aa, Homo sapiens
MAAMAVGGAGGSRVSSGRDLNCVPEIADTLGAVAKQGFDFLCMPVFHPRFKREFIQEPAKNRPGPQTRSDLLLSGRDWNTLIVGKLSPWIRPDSKVEKIRRNSEAAMLQELNFGAYLGLPAFLLPLNQEDNTNLARVLTNHIHTGHHSSMFWMRVPLVAPEDLRDDIIENAPTTHTEEYSGEEKTWMWWHNFRTLCDYSKRIAVALEIGADLPSNHVIDRWLGEPIKAAILPTSIFLTNKKGFPVLSKMHQRLIFRLLKLEVQFIITGTNHHSEKEFCSYLQYLEYLSQNRPPPNAYELFAKGYEDYLQSPLQPLMDNLESQTYEVFEKDPIKYSQYQQAIYKCLLDRVPEEEKDTNVQVLMVLGAGRGPLVNASLRAAKQADRRIKLYAVEKNPNAVVTLENWQFEEWGSQVTVVSSDMREWVAPEKADIIVSELLGSFADNELSPECLDGAQHFLKDDGVSIPGEYTSFLAPISSSKLYNEVRACREKDRDPEAQFEMPYVVRLHNFHQLSAPQPCFTFSHPNRDPMIDNNRYCTLEFPVEVNTVLHGFAGYFETVLYQDITLSIRPETHSPGMFSWFPILFPIKQPITVREGQTICVRFWRCSNSKKVWYEWAVTAPVCSAIHNPTGRSYTIGL

SEQ ID NO: 2 - protein, 620 aa, Homo sapiens
MRGPNSGTEKGRLVIPEKQGFDFLCMPVFHPRFKREFIQEPAKNRPGPQTRSDLLLSGRDWNTLIVGKLSPWIRPDSKVEKIRRNSEAAMLQELNFGAYLGLPAFLLPLNQEDNTNLARVLTNHIHTGHHSSMFWMRVPLVAPEDLRDDIIENAPTTHTEEYSGEEKTWMWWHNFRTLCDYSKRIAVALEIGADLPSNHVIDRWLGEPIKAAILPTSIFLTNKKGFPVLSKMHQRLIFRLLKLEVQFIITGTNHHSEKEFCSYLQYLEYLSQNRPPPNAYELFAKGYEDYLQSPLQPLMDNLESQTYEVFEKDPIKYSQYQQAIYKCLLDRVPEEEKDTNVQVLMVLGAGRGPLVNASLRAAKQADRRIKLYAVEKNPNAVVTLENWQFEEWGSQVTVVSSDMREWVAPEKADIIVSELLGSFADNELSPECLDGAQHFLKDDGVSIPGEYTSFLAPISSSKLYNEVRACREKDRDPEAQFEMPYVVRLHNFHQLSAPQPCFTFSHPNRDPMIDNNRYCTLEFPVEVNTVLHGFAGYFETVLYQDITLSIRPETHSPGMFSWFPILFPIKQPITVREGQTICVRFWRCSNSKKVWYEWAVTAPVCSAIHNPTGRSYTIGL

SEQ ID NO: 3 - protein, 16 aa, Artificial Sequence
  Synthetic Polypeptide
SGRGKGGKGLGKGGAK

SEQ ID NO: 4 - protein, 9 aa, Artificial Sequence
  Synthetic Polypeptide
MDYKDDDDK

SEQ ID NO: 5 - protein, 7 aa, Artificial Sequence
  Synthetic Polypeptide
MHHHHHH

SEQ ID NO: 6 - protein, 645 aa, Artificial Sequence
  Synthetic Polypeptide
MDYKDDDDKAAMAVGGAGGSRVSSGRDLNCVPEIADTLGAVAKQGFDFLCMPVFHPRFKREFIQEPAKNRPGPQTRSDLLLSGRDWNTLIVGKLSPWIRPDSKVEKIRRNSEAAMLQELNFGAYLGLPAFLLPLNQEDNTNLARVLTNHIHTGHHSSMFWMRVPLVAPEDLRDDIIENAPTTHTEEYSGEEKTWMWWHNFRTLCDYSKRIAVALEIGADLPSNHVIDRWLGEPIKAAILPTSIFLTNKKGFPVLSKMHQRLIFRLLKLEVQFIITGTNHHSEKEFCSYLQYLEYLSQNRPPPNAYELFAKGYEDYLQSPLQPLMDNLESQTYEVFEKDPIKYSQYQQAIYKCLLDRVPEEEKDTNVQVLMVLGAGRGPLVNASLRAAKQADRRIKLYAVEKNPNAVVTLENWQFEEWGSQVTVVSSDMREWVAPEKADIIVSELLGSFADNELSPECLDGAQHFLKDDGVSIPGEYTSFLAPISSSKLYNEVRACREKDRDPEAQFEMPYVVRLHNFHQLSAPQPCFTFSHPNRDPMIDNNRYCTLEFPVEVNTVLHGFAGYFETVLYQDITLSIRPETHSPGMFSWFPILFPIKQPITVREGQTICVRFWRCSNSKKVWYEWAVTAPVCSAIHNPTGRSYTIGL

SEQ ID NO: 7 - protein, 348 aa, Artificial Sequence
  Synthetic Polypeptide
MHHHHHHRKETPPPLVPPAAREWNLPPNAPACMERQLEAARYRSDGALLLGASSLSGRCWAGSLWLFKDPCAAPNEGFCSAGVQTEAGVADLTWVGERGILVASDSGAVELWELDENETLIVSKFCKYEHDDIVSTVSVLSSGTQAVSGSKDICIKVWDLAQQVVLSSYRAHAAQVTCVAASPHKDSVFLSCSEDNRILLWDTRCPKPASQIGCSAPGYLPTSLAWHPQQSEVFVFGDENGTVSLVDTKSTSCVLSSAVHSQCVTGLVFSPHSVPFLASLSEDCSLAVLDSSLSELFRSQAHRDFVRDATWSPLNHSLLTTVGWDHQVVHHVVPTEPLPAPGPASVTE

(19) United States
(12) Patent Application Publication
(10) Pub. No.: US 2017/0210751 A1
(43) Pub. Date: Jul. 27, 2017

(54) PRMT5 INHIBITORS AND USES THEREOF

(71) Applicant: Epizyme, Inc., Cambridge, MA (US)

(72) Inventors: Kenneth W. Duncan, Westwood, MA (US); Richard Chesworth, Concord, MA (US); Michael John Munchhol, Salem, CT (US); Gideon Shapiro, Gainesville, FL (US)

(73) Assignee: Epizyme, Inc., Cambridge, MA (US)

(21) Appl. No.: 15/321,270

(22) PCT Filed: Jun. 25, 2015
(86) PCT No.: PCT/US15/37759

§ 371 Date: Dec. 22, 2016

Related U.S. Application Data
(60) Provisional application No. 62/017,097, filed on Jun. 25, 2014.

(51) Int. Cl. C07D 495/04; C07D 487/04; C07D 498/04; C07D 513/04; C07D 471/04

(52) U.S. Cl. CPC C07D 495/04; C07D 513/04; C07D 471/04; C07D 498/04; C07D 487/04

(57) ABSTRACT

Described herein are compounds of Formula (A), pharmaceutically acceptable salts thereof, and pharmaceutical compositions thereof: wherein Y1 is of formula (x) or formula (y):Ring Y is a 5- to 6-membered heteroaryl ring; and V₄, V₅, R^{x}, x, y, and n are as defined herein. Compounds of the present invention are useful for inhibiting PRMT5 activity. Methods of using the compounds for treating PRMT5-mediated disorders are also described.

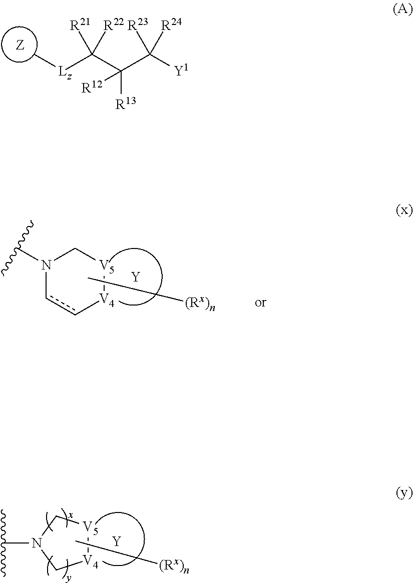

The present application claims priority under 35 U.S.C. §119(e) to U.S. provisional patent application, U.S. Ser. No. 62/017,097, filed Jun. 25, 2014, the entire contents of which is incorporated herein by reference.

### BACKGROUND OF THE INVENTION

Epigenetic regulation of gene expression is an important biological determinant of protein production and cellular differentiation and plays a significant pathogenic role in a number of human diseases.

Epigenetic regulation involves heritable modification of genetic material without changing its nucleotide sequence. Typically, epigenetic regulation is mediated by selective and reversible modification (e.g., methylation) of DNA and proteins (e.g., histones) that control the conformational transition between transcriptionally active and inactive states of chromatin. These covalent modifications can be controlled by enzymes such as methyltransferases (e.g., PRMT5), many of which are associated with specific genetic alterations that can cause human disease.

Disease-associated chromatin-modifying enzymes (e.g., PRMT5) play a role in diseases such as proliferative disorders, metabolic disorders, and blood disorders. Thus, there is a need for the development of small molecules that are capable of inhibiting the activity of PRMT5.

### DETAILED DESCRIPTION OF CERTAIN EMBODIMENTS

Protein arginine methyltransferase 5 (PRMT5) catalyzes the addition of two methyl groups to the two ω-guanidino nitrogen atoms of arginine, resulting in ω-NG, N′G symmetric dimethylation of arginine (sDMA) of the target protein. PRMT5 functions in the nucleus as well as in the cytoplasm, and its substrates include histones, spliceosomal proteins, transcription factors (See e.g., Sun et al., 2011, PNAS 108: 20538-20543). PRMT5 generally functions as part of a molecule weight protein complex. While the protein complexes of PRMT5 can have a variety of components, they generally include the protein MEP50 (methylosome protein 50). In addition, PRMT5 acts in conjunction with cofactor SAM (S-adenosyl methionine).

PRMT5 is an attractive target for modulation given its role in the regulation of diverse biological processes. It has now been found that compounds described herein, and pharmaceutically acceptable salts and compositions thereof, are effective as inhibitors of PRMT5. Such compounds have the general Formula (A):

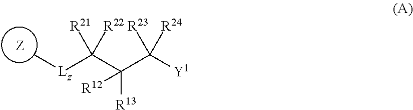

or a pharmaceutically acceptable salt thereof, wherein [symbol] , Ring Z, L_{z}, R²¹, R²², R²³, R²⁴, and Y¹ are as defined herein, R¹² is hydrogen, halogen, or optionally substituted C₁₋₃alkyl, and R¹³ is hydrogen, halogen, optionally substituted C₁₋₃alkyl, or OR¹, wherein R¹ is as defined herein.

In some embodiments, pharmaceutical compositions are provided which comprise a compound described herein (e.g., a compound of Formula (A)), or a pharmaceutically acceptable salt thereof, and optionally a pharmaceutically acceptable excipient.

In certain embodiments, compounds described herein inhibit activity of PRMT5. In certain embodiments, methods of inhibiting PRMT5 are provided which comprise contacting PRMT5 with an effective amount of a compound of Formula (A), or a pharmaceutically acceptable salt thereof. The PRMT5 may be purified or crude, and may be present in a cell, tissue, or a subject. Thus, such methods encompass inhibition of PRMT5 activity both in vitro and in vivo. In certain embodiments, the PRMT5 is wild-type PRMT5. In certain embodiments, the PRMT5 is overexpressed. In certain embodiments, the PRMT5 is a mutant. In certain embodiments, the PRMT5 is in a cell. In certain embodiments, the PRMT5 is in an animal, e.g., a human. In some embodiments, the PRMT5 is in a subject that is susceptible to normal levels of PRMT5 activity due to one or more mutations associated with a PRMT5 substrate. In some embodiments, the PRMT5 is in a subject known or identified as having abnormal PRMT5 activity (e.g., overexpression). In some embodiments, a provided compound is selective for PRMT5 over other methyltransferases. In certain embodiments, a provided compound is at least about 10-fold selective, at least about 20-fold selective, at least about 30-fold selective, at least about 40-fold selective, at least about 50-fold selective, at least about 60-fold selective, at least about 70-fold selective, at least about 80-fold selective, at least about 90-fold selective, or at least about 100-fold selective relative to one or more other methyltransferases.

In certain embodiments, methods of altering gene expression in a cell are provided which comprise contacting a cell with an effective amount of a compound of Formula (A), or a pharmaceutically acceptable salt thereof, or a pharmaceutical composition thereof. In certain embodiments, the cell in culture in vitro. In certain embodiments, cell is in an animal, e.g., a human.

In certain embodiments, methods of altering transcription in a cell are provided which comprise contacting a cell with an effective amount of a compound of Formula (A), or a pharmaceutically acceptable salt thereof, or a pharmaceutical composition thereof. In certain embodiments, the cell in culture in vitro. In certain embodiments, the cell is in an animal, e.g., a human.

In some embodiments, methods of treating a PRMT5-mediated disorder are provided which comprise administering to a subject suffering from a PRMT5-mediated disorder an effective amount of a compound described herein (e.g., a compound of Formula (A)), or a pharmaceutically acceptable salt thereof, or a pharmaceutical composition thereof. In certain embodiments, the PRMT5-mediated disorder is a proliferative disorder, a metabolic disorder, or a blood disorder. In certain embodiments, compounds described herein are useful for treating cancer. In certain embodiments, compounds described herein are useful for treating hematopoietic cancer, lung cancer, prostate cancer, melanoma, or pancreatic cancer. In certain embodiments, compounds described herein are useful for treating a hemoglobinopathy. In certain embodiments, compounds described herein are useful for treating sickle cell anemia. In certain embodiments, compounds described herein are useful for treating diabetes or obesity.

Compounds described herein are also useful for the study of PRMT5 in biological and pathological phenomena, the study of intracellular signal transduction pathways mediated by PRMT5, and the comparative evaluation of new PRMT5 inhibitors.

This application refers to various issued patent, published patent applications, journal articles, and other publications, all of which are incorporated herein by reference.

Definitions of specific functional groups and chemical terms are described in more detail below. The chemical elements are identified in accordance with the Periodic Table of the Elements, CAS version, Handbook of Chemistry and Physics, 75^{th} inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw Hill, N Y, 1962); and Wilen, Tables of Resolving Agents and Optical Resolutions p. 268 (E. L. Eliel, Ed., Univ. of Notre Dame Press, Notre Dame, Ind. 1972). The present disclosure additionally encompasses compounds described herein as individual isomers substantially free of other isomers, and alternatively, as mixtures of various isomers.

It is to be understood that the compounds of the present invention may be depicted as different tautomers. It should also be understood that when compounds have tautomeric forms, all tautomeric forms are intended to be included in the scope of the present invention, and the naming of any compound described herein does not exclude any tautomer form.

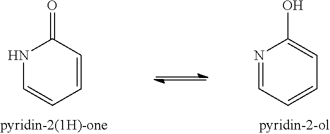 [SMILES: O=c1cccc[nH]1 ; Oc1ccccn1]

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such compounds are useful, for example, as analytical tools or probes in biological assays.

The term “aliphatic,” as used herein, includes both saturated and unsaturated, nonaromatic, straight chain (i.e., unbranched), branched, acyclic, and cyclic (i.e., carbocyclic) hydrocarbons. In some embodiments, an aliphatic group is optionally substituted with one or more functional groups. As will be appreciated by one of ordinary skill in the art, “aliphatic” is intended herein to include alkyl, alkenyl, alkynyl, cycloalkyl, and cycloalkenyl moieties.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Alkyl” refers to a radical of a straight-chain or branched saturated hydrocarbon group having from 1 to 20 carbon atoms (“C₁₋₂₀ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. In certain embodiments, each instance of an alkyl group is independently optionally substituted, e.g., unsubstituted (an “unsubstituted alkyl”) or substituted (a “substituted alkyl”) with one or more substituents. In certain embodiments, the alkyl group is unsubstituted C₁₋₁₀ alkyl (e.g., —CH₃). In certain embodiments, the alkyl group is substituted C₁₋₁₀ alkyl.

In some embodiments, an alkyl group is substituted with one or more halogens. “Perhaloalkyl” is a substituted alkyl group as defined herein wherein all of the hydrogen atoms are independently replaced by a halogen, e.g., fluoro, bromo, chloro, or iodo. In some embodiments, the alkyl moiety has 1 to 8 carbon atoms (“C₁₋₈ perhaloalkyl”). In some embodiments, the alkyl moiety has 1 to 6 carbon atoms (“C₁₋₆ perhaloalkyl”). In some embodiments, the alkyl moiety has 1 to 4 carbon atoms (“C₁₋₄ perhaloalkyl”). In some embodiments, the alkyl moiety has 1 to 3 carbon atoms (“C₁₋₃ perhaloalkyl”). In some embodiments, the alkyl moiety has 1 to 2 carbon atoms (“C₁₋₂ perhaloalkyl”). In some embodiments, all of the hydrogen atoms are replaced with fluoro. In some embodiments, all of the hydrogen atoms are replaced with chloro. Examples of perhaloalkyl groups include —CF₃, —CF₂CF₃, —CF₂CF₂CF₃, —CCl₃, —CFCl₂, —CF₂Cl, and the like.

“Alkenyl” refers to a radical of a straight-chain or branched hydrocarbon group having from 2 to 20 carbon atoms, one or more carbon-carbon double bonds, and no triple bonds (“C₂₋₂₀ alkenyl”). In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1 butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. In certain embodiments, each instance of an alkenyl group is independently optionally substituted, e.g., unsubstituted (an “unsubstituted alkenyl”) or substituted (a “substituted alkenyl”) with one or more substituents. In certain embodiments, the alkenyl group is unsubstituted C₂₋₁₀ alkenyl. In certain embodiments, the alkenyl group is substituted C₂₋₁₀ alkenyl.

“Alkynyl” refers to a radical of a straight-chain or branched hydrocarbon group having from 2 to 20 carbon atoms, one or more carbon-carbon triple bonds, and optionally one or more double bonds (“C₂₋₂₀ alkynyl”). In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. In certain embodiments, each instance of an alkynyl group is independently optionally substituted, e.g., unsubstituted (an “unsubstituted alkynyl”) or substituted (a “substituted alkynyl”) with one or more substituents. In certain embodiments, the alkynyl group is unsubstituted C₂₋₁₀ alkynyl. In certain embodiments, the alkynyl group is substituted C₂₋₁₀ alkynyl.

“Carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or is a fused, bridged or spiro-fused ring system such as a bicyclic system (“bicyclic carbocyclyl”) and can be saturated or can be partially unsaturated. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. In certain embodiments, each instance of a carbocyclyl group is independently optionally substituted, e.g., unsubstituted (an “unsubstituted carbocyclyl”) or substituted (a “substituted carbocyclyl”) with one or more substituents. In certain embodiments, the carbocyclyl group is unsubstituted C₃₋₁₀ carbocyclyl. In certain embodiments, the carbocyclyl group is a substituted C₃₋₁₀ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). In certain embodiments, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl.

“Heterocyclyl” or “heterocyclic” refers to a radical of a 3 to 14-membered non aromatic ring system having ring carbon atoms and 1 to 4 ring heteroatoms, wherein each heteroatom is independently selected from nitrogen, oxygen, and sulfur (“3-14-membered heterocyclyl”). In certain embodiments, heterocyclyl or heterocyclic refers to a radical of a 3-10 membered nonaromatic ring system having ring carbon atoms and 1-4 ring heteroatoms, wherein each heteroatom is independently selected from nitrogen, oxygen, and sulfur (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro-fused ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. In certain embodiments, each instance of heterocyclyl is independently optionally substituted, e.g., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered nonaromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered nonaromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered nonaromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms independently selected from nitrogen, oxygen, and sulfur. In some embodiments, the 5-6 membered heterocyclyl has 1-2 ring heteroatoms independently selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, and thiorenyl. Exemplary 4-membered heterocyclyl groups containing one heteroatom include, without limitation, azetidinyl, oxetanyl, and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5 membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, and dioxanyl. Exemplary 6 membered heterocyclyl groups containing three heteroatoms include, without limitation, triazinanyl, oxadiazinanyl, thiadiazinanyl, oxathiazinanyl, and dioxazinanyl. Exemplary 7 membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl, and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. In certain embodiments, each instance of an aryl group is independently optionally substituted, e.g., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

“Heteroaryl” refers to a radical of a 5-14 membered monocyclic or polycyclic (e.g., bicyclic or tricyclic) 4n+2 aromatic ring system (e.g., having 6 or 10π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In certain embodiments, heteroaryl refers to a radical of a 5-10 membered monocyclic or bicyclic 4n+2 aromatic ring system having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl).

In some embodiments, a heteroaryl group is a 5-14 membered aromatic ring system having ring carbon atoms and 1-4 ring heteroatoms provided in the aromatic ring system, wherein each heteroatom is independently selected from nitrogen, oxygen, and sulfur (“5-14 membered heteroaryl”). In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms independently selected from nitrogen, oxygen, and sulfur. In some embodiments, the 5-6 membered heteroaryl has 1-2 ring heteroatoms independently selected from nitrogen, oxygen, and sulfur. In some embodiments, the 5-6 membered heteroaryl has 1 ring heteroatom selected from nitrogen, oxygen, and sulfur. In certain embodiments, each instance of a heteroaryl group is independently optionally substituted, e.g., unsubstituted (“unsubstituted heteroaryl”) or substituted (“substituted heteroaryl”) with one or more substituents. In certain embodiments, the heteroaryl group is unsubstituted 5-14 membered heteroaryl. In certain embodiments, the heteroaryl group is substituted 5-14 membered heteroaryl.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

“Fused” or “ortho-fused” are used interchangeably herein, and refer to two rings that have two atoms and one bond in common, e.g.,

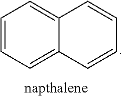

“Bridged” refers to a ring system containing (1) a bridgehead atom or group of atoms which connect two or more non-adjacent positions of the same ring; or (2) a bridgehead atom or group of atoms which connect two or more positions of different rings of a ring system and does not thereby form an ortho-fused ring, e.g.,

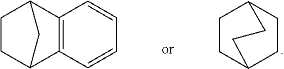 [SMILES: c1ccc2c(c1)C1CCC2C1 ; C1CC2CCC1CC2]

“Spiro” or “Spiro-fused” refers to a group of atoms which connect to the same atom of a carbocyclic or heterocyclic ring system (geminal attachment), thereby forming a ring, e.g.,

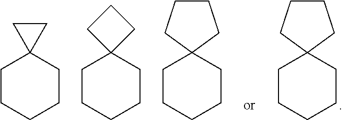

Spiro-fusion at a bridgehead atom is also contemplated.

“Partially unsaturated” refers to a group that includes at least one double or triple bond. The term “partially unsaturated” is intended to encompass rings having multiple sites of unsaturation, but is not intended to include aromatic groups (e.g., aryl or heteroaryl groups) as herein defined. Likewise, “saturated” refers to a group that does not contain a double or triple bond, i.e., contains all single bonds.

In some embodiments, aliphatic, alkyl, alkenyl, alkynyl, carbocyclyl, heterocyclyl, aryl, and heteroaryl groups, as defined herein, are optionally substituted (e.g., “substituted” or “unsubstituted” aliphatic, “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, including any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂, —NR^{bb}P(═O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups;

or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};

each instance of R^{aa} is, independently, selected from C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S;

each instance of R^{ee} is, independently, selected from C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups;

each instance of R^{ff} is, independently, selected from hydrogen, C₁₋₆ alkyl, C₁₋₆ perhaloalkyl, C₂₋₆ alkenyl, C₂₋₆ alkynyl, C₃₋₁₀ carbocyclyl, 3-10 membered heterocyclyl, C₆-10 aryl and 5-10 membered heteroaryl, or two R^{ff} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{gg} groups; and

each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂ (C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)₂(C₁₋₆ alkyl), —P(═O)(C₁₋₆ alkyl)₂, —OP(═O)(C₁₋₆ alkyl)₂, —OP(═O)(OC₁₋₆ alkyl)₂, C₁ alkyl, C₁₋₆ perhaloalkyl, C₂₋₆ alkenyl, C₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻is a counterion.

A “counterion” or “anionic counterion” is a negatively charged group associated with a cationic quaternary amino group in order to maintain electronic neutrality. Exemplary counterions include halide ions (e.g., F⁻, Cl⁻, Br⁻, I⁻), NO₃⁻, ClO₄⁻, OH⁻, H₂PO₄⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

“Halo” or “halogen” refers to fluorine (fluoro, —F), chlorine (chloro, —Cl), bromine (bromo, —Br), or iodine (iodo, —I).

Nitrogen atoms can be substituted or unsubstituted as valency permits, and include primary, secondary, tertiary, and quarternary nitrogen atoms. Exemplary nitrogen atom substitutents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})², —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on a nitrogen atom is a nitrogen protecting group (also referred to as an amino protecting group). Nitrogen protecting groups include, but are not limited to, —OH, —OR^{aa}, —N(R^{cc})², —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc}, and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Amide nitrogen protecting groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine, o-nitrobenzamide, and o-(benzoyloxymethyl)benzamide.

Carbamate nitrogen protecting groups (e.g., —C(═O)OR^{aa}) include, but are not limited to, methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Sulfonamide nitrogen protecting groups (e.g., —S(═O)₂R^{aa}) include, but are not limited to, p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N—1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to as a hydroxyl protecting group). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, Si(R^{aa})₃, —P(R^{cc})₂, —P(R^{cc})₃, —P(═O)₂R^{aa}, —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{CC} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodisulfuran-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), t-butyl carbonate (BOC), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on a sulfur atom is a sulfur protecting group (also referred to as a thiol protecting group). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃, —P(═O)₂R^{aa}, —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{CC} are as defined herein. Sulfur protecting groups are well known in the art and include those described in detail in Protecting Groups in Organic Synthesis, T. W. Greene and P. G. M. Wuts, 3rd edition, John Wiley & Sons, 1999, incorporated herein by reference.

As used herein, a “leaving group”, or “LG”, is a term understood in the art to refere to a molecular fragment that departs with a pair of electrons upon heterolytic bond cleavage, wherein the molecular fragment is an anion or neutral molecule. See, for example, Smith, March Advanced Organic Chemistry 6th ed. (501-502). Examples of suitable leaving groups include, but are not limited to, halides (such as chloride, bromide, or iodide), alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, haloformates, —NO₂, trialkylammonium, and aryliodonium salts. In some embodiments, the leaving group is a sulfonic acid ester. In some embodiments, the sulfonic acid ester comprises the formula —OSO₂R^{LG1} wherein R^{LG1} is selected from the group consisting alkyl optionally, alkenyl optionally substituted, heteroalkyl optionally substituted, aryl optionally substituted, heteroaryl optionally substituted, arylalkyl optionally substituted, and heterarylalkyl optionally substituted. In some embodiments, R^{LG1} is substituted or unsubstituted C₁-C₆ alkyl. In some embodiments, R^{LG1} is methyl. In some embodiments, R^{LG1} is —CF₃. In some embodiments, R^{LG1} is substituted or unsubstituted aryl. In some embodiments, R^{LG1} is substituted or unsubstituted phenyl. In some embodiments R^{LG1} is:

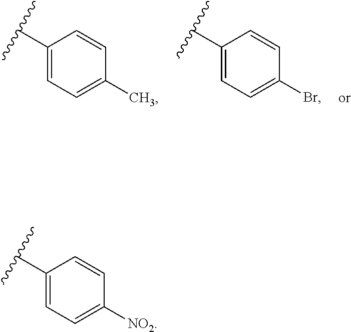 [SMILES: *c1ccc(C)cc1 ; *c1ccc(Br)cc1 ; "or" ; *c1ccc([N+](=O)[O-])cc1]

In some cases, the leaving group is toluenesulfonate (tosylate, Ts), methanesulfonate (mesylate, Ms), p-bromobenzenesulfonyl (brosylate, Bs), or trifluoromethanesulfonate (triflate, Tf). In some cases, the leaving group is a brosylate (p-bromobenzenesulfonyl). In some cases, the leaving group is a nosylate (2-nitrobenzenesulfonyl). In some embodiments, the leaving group is a sulfonate-containing group. In some embodiments, the leaving group is a tosylate group. The leaving group may also be a phosphineoxide (e.g., formed during a Mitsunobu reaction) or an internal leaving group such as an epoxide or cyclic sulfate.

These and other exemplary substituents are described in more detail in the Detailed Description, Examples, and claims. The present disclosure is not intended to be limited in any manner by the above exemplary listing of substituents.

“Pharmaceutically acceptable salt” refers to those salts which are, within the scope of sound medical judgment, suitable for use in contact with the tissues of humans and other animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences (1977) 66:1-19. Pharmaceutically acceptable salts of the compounds describe herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid, or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N+(C₁₋₄alkyl)₄ salts. Representative alkali or alkaline earth metal salts include sodium, lithium, potassium, calcium, magnesium, and the like. Further pharmaceutically acceptable salts include, when appropriate, quaternary salts.

A “subject” to which administration is contemplated includes, but is not limited to, humans (e.g., a male or female of any age group, e.g., a pediatric subject (e.g, infant, child, adolescent) or adult subject (e.g., young adult, middle-aged adult or senior adult)) and/or other non-human animals, for example, non-human mammals (e.g., primates (e.g., cynomolgus monkeys, rhesus monkeys); commercially relevant mammals such as cattle, pigs, horses, sheep, goats, cats, and/or dogs), birds (e.g., commercially relevant birds such as chickens, ducks, geese, and/or turkeys), rodents (e.g., rats and/or mice), reptiles, amphibians, and fish. In certain embodiments, the non-human animal is a mammal. The non-human animal may be a male or female at any stage of development. A non-human animal may be a transgenic animal.

“Condition,” “disease,” and “disorder” are used interchangeably herein.

“Treat,” “treating” and “treatment” encompasses an action that occurs while a subject is suffering from a condition which reduces the severity of the condition or retards or slows the progression of the condition (“therapeutic treatment”). “Treat,” “treating” and “treatment” also encompasses an action that occurs before a subject begins to suffer from the condition and which inhibits or reduces the severity of the condition (“prophylactic treatment”).

An “effective amount” of a compound refers to an amount sufficient to elicit the desired biological response, e.g., treat the condition. As will be appreciated by those of ordinary skill in this art, the effective amount of a compound described herein may vary depending on such factors as the desired biological endpoint, the pharmacokinetics of the compound, the condition being treated, the mode of administration, and the age and health of the subject. An effective amount encompasses therapeutic and prophylactic treatment.

A “therapeutically effective amount” of a compound is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms or causes of the condition, or enhances the therapeutic efficacy of another therapeutic agent.

A “prophylactically effective amount” of a compound is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

As used herein, the term “methyltransferase” represents transferase class enzymes that are able to transfer a methyl group from a donor molecule to an acceptor molecule, e.g., an amino acid residue of a protein or a nucleic base of a DNA molecule. Methytransferases typically use a reactive methyl group bound to sulfur in S-adenosyl methionine (SAM) as the methyl donor. In some embodiments, a methyltransferase described herein is a protein methyltransferase. In some embodiments, a methyltransferase described herein is a histone methyltransferase. Histone methyltransferases (HMT) are histone-modifying enzymes, (including histone-lysine N-methyltransferase and histone-arginine N-methyltransferase), that catalyze the transfer of one or more methyl groups to lysine and arginine residues of histone proteins. In certain embodiments, a methyltransferase described herein is a histone-arginine N-methyltransferase.

As generally described above, provided herein are compounds useful as PRMT5 inhibitors. In some embodiments, the present disclosure provides a compound of Formula (A):

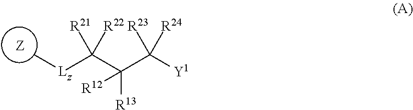

or a pharmaceutically acceptable salt thereof,
wherein:

R¹² is hydrogen, halogen, or optionally substituted C₁₋₃alkyl;

R¹³ is hydrogen, halogen, optionally substituted C₁₋₃alkyl, —NR^{A1}R^{A2}, or —OR¹;

R^{A1} and R^{A2} are each independently hydrogen, optionally substituted C₁₋₃ alkyl, a nitrogen protecting group, or R^{A1} and R^{A2} are taken together with the intervening nitrogen atom to form an optionally substituted 3-6 membered heterocyclic ring;

R¹ is hydrogen, R^{z}, or —C(O)R^{z}, wherein R^{z} is optionally substituted C₁₋₆ alkyl;

L_{z} is a linker or is absent;

Ring Z is an optionally substituted, monocyclic or bicyclic, saturated, partially unsaturated, or aromatic ring having 0-4 heteroatoms independently selected from nitrogen, oxygen, and sulfur;

R²¹, R²², R²³, and R²⁴ are independently hydrogen, halo, or optionally substituted aliphatic;

Y¹ is of formula (x) or formula (y)

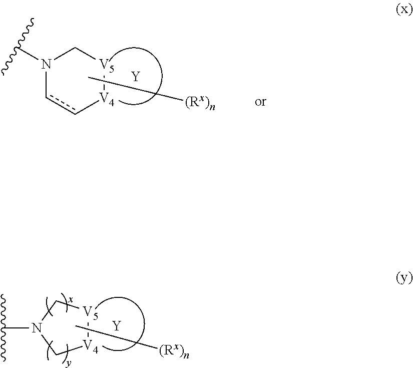

Ring Y is a 5- to 6-membered heteroaryl ring;

each instance of V₄ and V₅ is independently C or N;

each R^{x} is independently selected from the group consisting of halo, —CN, optionally substituted aliphatic, —OR′, —N(R″)₂, optionally substituted aryl, optionally substituted heteroaryl, and if attached to a nitrogen atom, a nitrogen protecting group;

R′ is hydrogen or optionally substituted aliphatic;

R″ is hydrogen or optionally substituted aliphatic, or two R″ are taken together with their intervening atoms to form a heterocyclic ring;

n is 0, 1, 2, 3, 4, 5, 6, 7, or 8;

[symbol] corresponds to a single or double bond; and

x is 0 and y is 2, 3, or 4; or

x is 1 and y is 1; or

x is 1 and y is 3.

In some embodiments, the carbon attached to R¹² has (S)-stereochemistry. In some embodiments, the carbon attached to R¹² has (R)-stereochemistry. In some embodiments, the carbon attached to R¹³ has (S)-stereochemistry. In some embodiments, the carbon attached to R¹³ has (R) stereochemistry.

As generally defined above, R¹² is hydrogen, halogen, or optionally substituted C₁₋₃alkyl. In certain embodiments, R¹² is hydrogen. In certain embodiments, R¹² is optionally substituted C₁₋₃alkyl, e.g., optionally substituted with halogen. In certain embodiments, R¹² is optionally substituted C₁alkyl, e.g., methyl or trifluoromethyl. In certain embodiments, R¹² is optionally substituted C₂ alkyl, e.g., ethyl. In certain embodiments, R¹² is optionally substituted C₃ alkyl, e.g., propyl. In certain embodiments, R¹² is fluoro, provided that R¹³ is not —OR¹. In certain embodiments, R¹² is chloro, provided that R¹³ is not —OR¹. In certain embodiments, R¹² is bromo, provided that R¹³ is not —OR¹. In certain embodiments, R¹² is iodo, provided that R¹³ is not —OR¹.

As generally defined above, R¹³ is hydrogen, halogen, optionally substituted C₁₋₃alkyl, —NR^{A1}R^{A2} or —OR¹. In certain embodiments, R¹³ is hydrogen. In certain embodiments, R¹³ is optionally substituted C₁₋₃alkyl, e.g., optionally substituted with halogen. In certain embodiments, R¹³ is optionally substituted C₁alkyl, e.g., methyl or trifluoromethyl. In certain embodiments, R¹³ is optionally substituted C₂ alkyl, e.g., ethyl. In certain embodiments, R¹³ is optionally substituted C₃ alkyl, e.g., propyl. In certain embodiments, R¹³ is fluoro. In certain embodiments, R¹³ is chloro. In certain embodiments, R¹³ is bromo. In certain embodiments, R¹³ is iodo.

In some embodiments, both R¹² and R¹³ are optionally substituted C₁₋₃alkyl. In some embodiments, R¹² is halogen e.g., fluoro, bromo, chloro, or iodo, provided that R¹³ is not —OR¹. In some embodiments, R¹³ is halogen e.g., fluoro, bromo, chloro, or iodo. In some embodiments, both R¹² and R¹³ are halogen e.g., fluoro, bromo, chloro, or iodo. In some embodiments, R¹² is halogen e.g., fluoro, bromo, chloro, or iodo and R¹³ is optionally substituted C₁₋₃alkyl. In some embodiments, R¹² is optionally substituted C₁₋₃alkyl and R¹³ is halogen e.g., fluoro, bromo, chloro, or iodo. In some embodiments, R¹³ is —OR¹. In some embodiments, R¹² is optionally substituted C₁₋₃alkyl and R¹³ is —OR¹. In some embodiments, R¹² is hydrogen and R¹³ is —OR¹. In some embodiments, R¹² is hydrogen and R¹³ optionally substituted C₁₋₃alkyl. In some embodiments, R¹² is optionally substituted C₁₋₃alkyl and R¹³ is hydrogen. In some embodiments, R¹² is halogen e.g., fluoro, bromo, chloro, or iodo and R¹³ is hydrogen. In some embodiments, R¹² is hydrogen and R¹³ is halogen e.g., fluoro, bromo, chloro, or iodo.

In some embodiments of Formula (A), wherein R¹² is hydrogen, the present disclosure provides a compound of Formula (A-1) or Formula (A-1′)

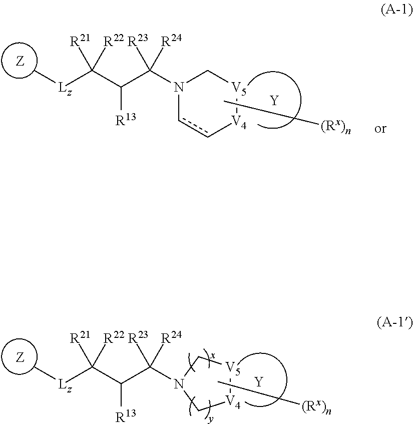

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In some embodiments of Formula (A), wherein both R¹² and R¹³ are hydrogen, the present disclosure provides a compound of Formula (A-2) or Formula (A-2′):

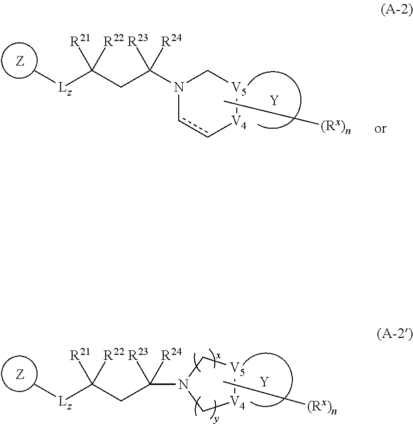

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In some embodiments of Formula (A), wherein R¹³ is —OR¹, the present disclosure provides a compound of Formula (A-3) or Formula (A-3′):

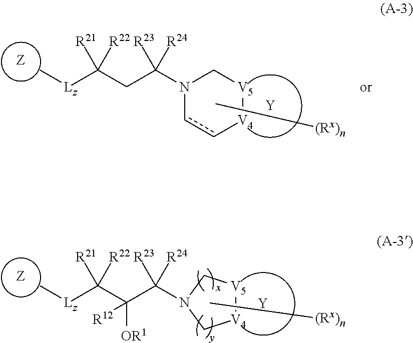

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In some embodiments of Formula (A), wherein R¹² is hydrogen and R¹³ is —OR¹, the present disclosure provides a compound of Formula (I) or Formula (I′):

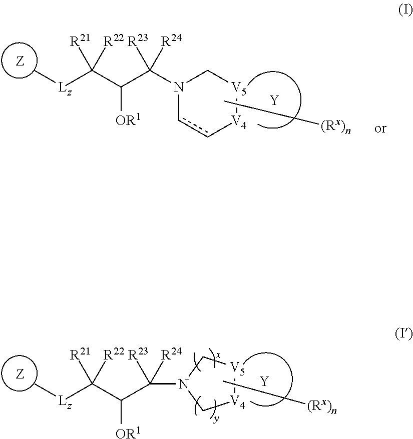

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

As defined generally above, L_{z} is a linker or is absent. For example, in certain embodiments, L_{z} is a linker —X_{A}—C(R^{2A})(R^{3A})C(═O)N(R)—, a linker L_{B} as defined herein, or a linker L_{D} as defined herein. Alternatively, in certain embodiments, L_{z} is absent, and the carbon substituted with R²¹ and R²² is directly attached to Ring Z.

In certain embodiments, L_{z} is a linker —X_{A}—C(R^{2A})(R^{3A})C(═O)N(R)— and Ring Z is a group Cy^{A}, as defined herein.

In certain embodiments, L_{z} is a linker L_{B} and Ring Z is a group Ar, as defined herein.

In certain embodiments, L_{z} is absent, and Ring Z is a group referred to herein as Ring C:

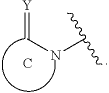

In certain embodiments, L_{z} is linker L_{D} (which encompasses linker L_{B} and other linkers) and Ring Z is a group referred to herein as Ring A:

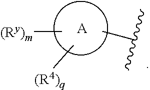

In certain embodiments of Formula (A), L_{z} is a linker —X_{A}—C(R^{2A})(R^{3A})C(═O)N(R)— and Ring Z is a group Cy^{A}, to provide a compound of Formula (A-I^{A}):

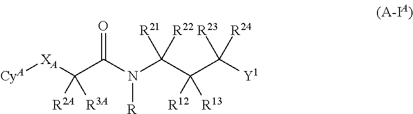

or a pharmaceutically acceptable salt thereof;
wherein:

X_{A} is a bond, —O—, —N(R)—, —CR^{4A}R^{5A}—, —O—CR^{4A}R^{5A}, —N(R)—CR^{4A}R^{5A}—, —O—CR^{4A}R^{5A}—O—, —N(R)—CR^{4A}R^{5A}—O, —N(R)—CR^{4A}R^{5A}—N(R)—, —O—CR^{4A}R^{5A}—N(R)—, —CR^{4A}R^{5A}—O—, —CR^{4A}R^{5A}—N(R)—, —O—CR^{4A}R^{5A}—CR^{6A}R^{7A}—, —N(R)—CR^{4A}R^{5A}—CR^{6A}R^{7A}—, —CR^{6A}R^{7A}—CR^{4A}R^{5A}—O—, —CR^{6A}R^{7A}—CR^{4A}R^{5A}—N(R)—, or —CR^{6A}R^{7A}—CR^{4A}R^{5A}—;

each R is independently hydrogen or optionally substituted C₁₋₆ aliphatic;

R^{4A} and R^{5A} are independently selected from the group consisting of hydrogen, halo, —CN, —NO₂, optionally substituted aliphatic, optionally substituted carbocyclyl, optionally substituted phenyl, optionally substituted heterocyclyl, optionally substituted heteroaryl, —OR^{A}, —N(R^{B})₂, —SR^{A}, —C(═O)R^{A}, —C(O)OR^{A}, —C(O)SR^{A}, —C(O)N(R^{B})₂, —C(O)N(R^{B})N(R^{B})₂, —OC(O)R^{A}, —OC(O)N(R^{B})₂, —NR^{B}C(O)R^{A}, —NR^{B}C(O)N(R^{B})₂, —NR^{B}C(O)N(R^{B})N(R^{B})₂, —NR^{B}C(O)OR^{A}, —SC(O)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(O)R^{A}, —OS(O)₂R^{A}, —SO₂R^{A}, —NR^{B}SO₂R^{A}, or —SO₂N(R^{B})₂; or R^{4A} and R^{5A} are taken together with their intervening atoms to form an optionally substituted carbocyclic or heterocyclic ring;

R^{6A} and R^{7A} are independently selected from the group consisting of hydrogen, halo, —CN, —NO₂, optionally substituted aliphatic, optionally substituted carbocyclyl, optionally substituted phenyl, optionally substituted heterocyclyl, optionally substituted heteroaryl, —OR^{A}, —N(R^{B})₂, —SR^{A}, —C(═O)R^{A}, —C(O)OR^{A}, —C(O)SR^{A}, —C(O)N(R^{B})₂, —C(O)N(R^{B})N(R^{B})₂, —OC(O)R^{A}, —OC(O)N(R^{B})₂, —NR^{B}C(O)R^{A}, —NR^{B}C(O)N(R^{B})₂, —NR^{B}C(O)N(R^{B})N(R^{B})₂, —NR^{B}C(O)OR^{A}, —SC(O)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(O)R^{A}, —OS(O)₂R^{A}, —SO₂R^{A}, —NR^{B}SO₂R^{A}, or —SO₂N(R^{B})₂; or R^{6A} and R^{7A} are taken together with their intervening atoms to form an optionally substituted carbocyclic or heterocyclic ring;

R^{2A} and R^{3A} are independently selected from the group consisting of hydrogen, halo, —CN, —NO₂, optionally substituted aliphatic, optionally substituted carbocyclyl, optionally substituted phenyl, optionally substituted heterocyclyl, optionally substituted heteroaryl, —OR^{A}, —N(R^{B})₂, —SR^{A}, —C(═O)R^{A}, —C(O)OR^{A}, —C(O)SR^{A}, —C(O)N(R^{B})₂, —C(O)N(R^{B})N(R^{B})₂, —OC(O)R^{A}, —OC(O)N(R^{B})₂, —NR^{B}C(O)R^{A}, —NR^{B}C(O)N(R^{B})₂, —NR^{B}C(O)N(R^{B})N(R^{B})₂, —NR^{B}C(O)OR^{A}, —SC(O)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(O)R^{A}, —OS(O)₂R^{A}, —SO₂R^{A}, —NR^{B}SO₂R^{A}, or —SO₂N(R^{B})₂; or R^{2A} and R^{3A} are taken together with their intervening atoms to form an optionally substituted carbocyclic or heterocyclic ring;

each R^{A} is independently selected from the group consisting of hydrogen, optionally substituted aliphatic, optionally substituted carbocyclyl, optionally substituted heterocyclyl, optionally substituted aryl, and optionally substituted heteroaryl;

each R^{B} is independently selected from the group consisting of hydrogen, optionally substituted aliphatic, optionally substituted carbocyclyl, optionally substituted heterocyclyl, optionally substituted aryl, and optionally substituted heteroaryl, or two R^{B} groups are taken together with their intervening atoms to form an optionally substituted heterocyclic ring;

Cy^{A} is a monocyclic or bicyclic, saturated, partially unsaturated, or aromatic ring having 0-4 heteroatoms independently selected from nitrogen, oxygen, and sulfur, wherein Cy^{A} is substituted with 0, 1, 2, 3, or 4 R^{y} groups; and

each R^{y} is independently selected from the group consisting of halo, —CN, —NO₂, optionally substituted aliphatic, optionally substituted carbocyclyl, optionally substituted aryl, optionally substituted heterocyclyl, optionally substituted heteroaryl, —OR^{A}, —N(R^{B})₂, —SR^{A}, —C(═O)R^{A}, —C(O)OR^{A}, —C(O)SR^{A}, —C(O)N(R^{B})₂, —C(O)N(R^{B})N(R^{B})₂, —OC(O)R^{A}, —OC(O)N(R^{B})₂, —NR^{B}C(O)R^{A}, —NR^{B}C(O)N(R^{B})₂, —NR^{B}C(O)N(R^{B})N(R^{B})₂, —NR^{B}C(O)OR^{A}, SC(O)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(O)R^{A}, —OS(O)₂R^{A}, —SO₂R^{A}, —NR^{B}SO₂R^{A}, or —SO₂N(R^{B})₂.

In certain embodiments of Formula (A-I^{A}), wherein R¹² is hydrogen, and R¹³ is —OR¹, a provided compound is of Formula (I^{A}) or Formula (I^{A′}):

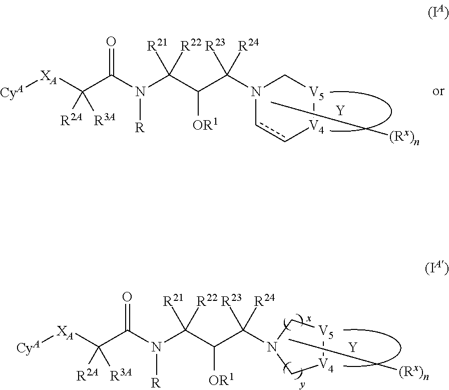

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (I^{A}) or Formula (I^{A′}), a provided compound is of Formula (I^{A}-a) or Formula (I^{A}-a′):

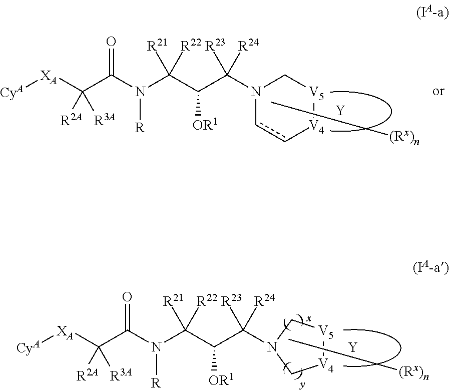

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (I^{A}) or Formula (I^{A′}), a provided compound is of Formula (I^{A}-b) or Formula (I^{A}-b′):

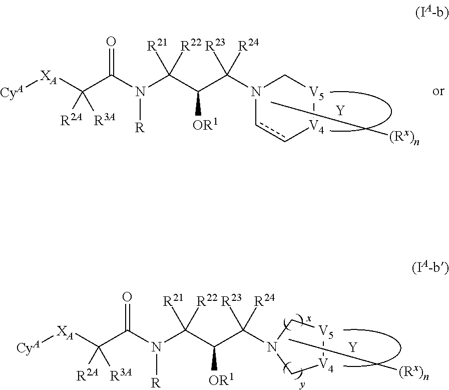

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (I^{A}) or Formula (I^{A′}), wherein R²¹-R²⁴ is hydrogen, a provided compound is of Formula (I^{A}-c) or Formula (I^{A}-c′):

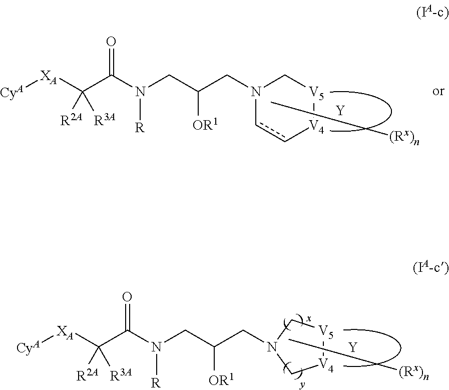

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (I^{A}) or Formula (I^{A′}), wherein R²¹-R²⁴ is hydrogen, a provided compound is of Formula (I^{A}-d) or Formula (I^{A}-d′):

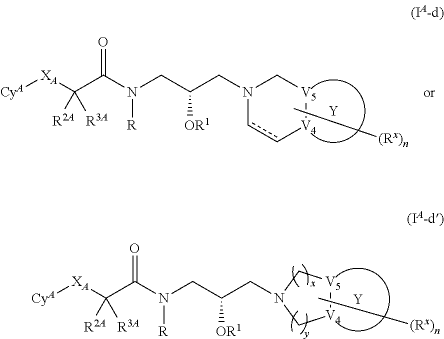

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (I^{A}) or Formula (I^{A′}), wherein R²¹-R²⁴ is hydrogen, a provided compound is of Formula (I^{A}-e) or Formula (I^{A}-e′):

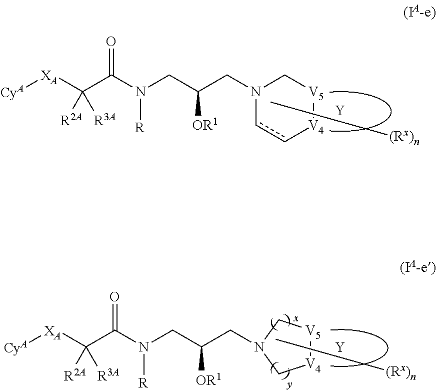

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (A-I^{A}), wherein X_{A} is —O— and R²¹-R²⁴ is hydrogen, a provided compound is of Formula (II^{A}) or Formula (II^{A′}):

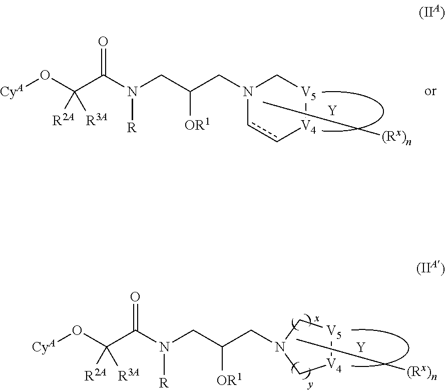

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (II^{A}) or Formula (II^{A′}), wherein X_{A} is —O— and R²¹-R²⁴ is hydrogen, a provided compound is of Formula (II^{A}-a) or Formula (II^{A}-a′):

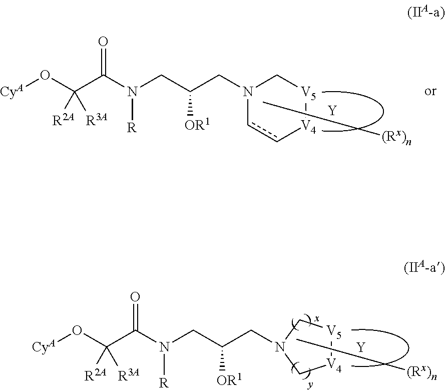

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (II^{A}) or Formula (II^{A′}), wherein X_{A} is —O— and R²¹-R²⁴ is hydrogen, a provided compound is of Formula (II^{A}-b) or Formula (II^{A}-b′):

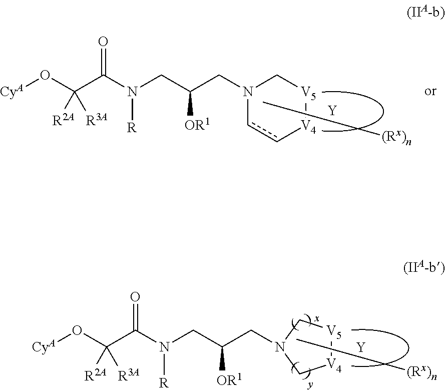

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (A-I^{A}), wherein X_{A} is —NR— and R²¹-R²⁴ is hydrogen, a provided compound is of Formula (III^{A}) or Formula (III^{A′}):

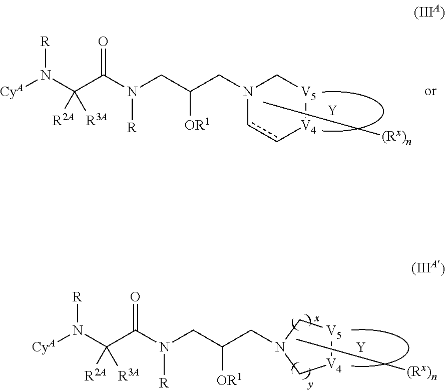

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (III^{A}) or Formula (III^{A′}), wherein X_{A} is —NR— and R²¹-R²⁴ is hydrogen, a provided compound is of Formula (III^{A}-a) or Formula (III^{A}-a′):

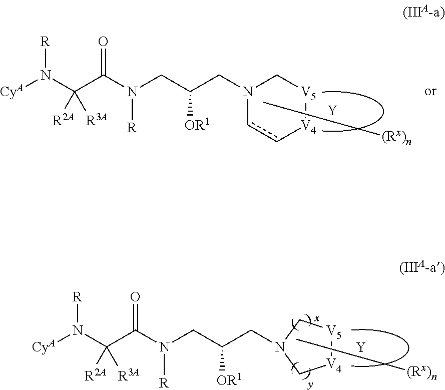

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (III^{A}) or Formula (III^{A′}), wherein X_{A} is —NR— and R²¹-R²⁴ is hydrogen, a provided compound is of Formula (III^{A}-b) or Formula (III^{A}-b′):

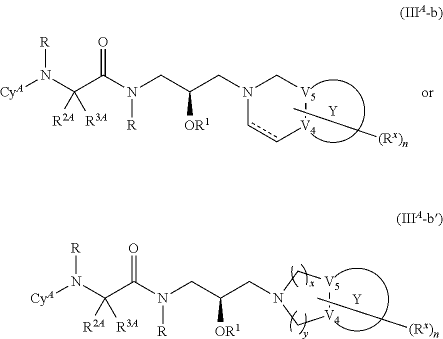

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (A-I^{A}), wherein X_{A} is —CR^{4A}R^{5A}— and R²¹-R²⁴ is hydrogen, a provided compound is of Formula (IV^{A}) or Formula (IV^{A′}):

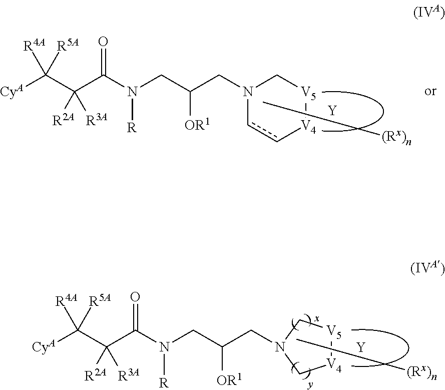

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (IV^{A}) or Formula (IV^{A′}), wherein X_{A} is —CR^{4A}R^{5A}— and R²¹-R²⁴ is hydrogen, a provided compound is of Formula (IV^{A}-a) or Formula (IV^{A}-a′):

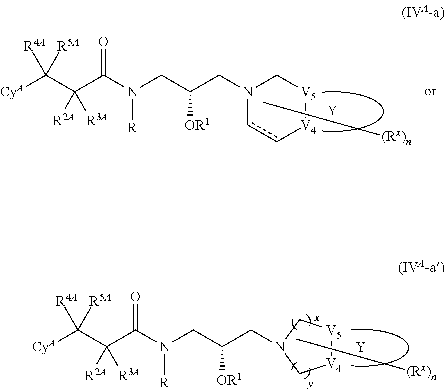

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (IV^{A}) or Formula (IV^{A′}), wherein X_{A} is —CR^{4A}R^{5A}— and R²¹-R²⁴ is hydrogen, a provided compound is of Formula (IV^{A}-b) or Formula (IV^{A}-b′):

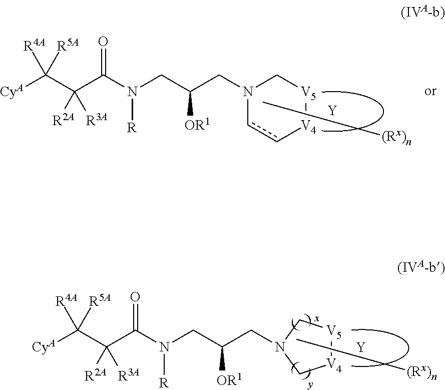

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (A-I^{A}), wherein X_{A} is a bond and R²¹R²⁴ is hydrogen, a provided compound is of Formula (V^{A}) or Formula (V^{A′}):

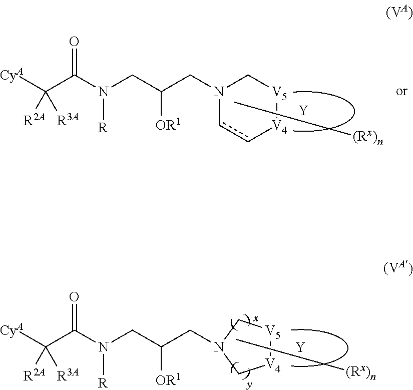

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{A}) or Formula (V^{A′}), wherein X_{A} is a bond and R²¹-R²⁴ is hydrogen, a provided compound is of Formula (V^{A}-a) or Formula (V^{A}-a′):

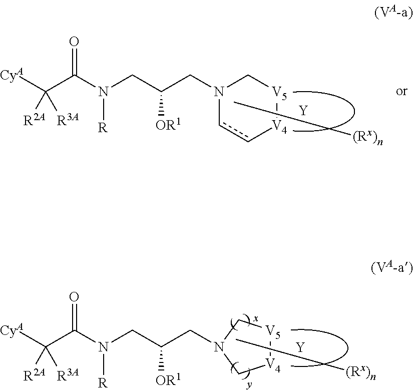

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{A}) or Formula (V^{A′}), wherein X_{A} is a bond and R²¹-R²⁴ is hydrogen, a provided compound is of Formula (V^{A}-b) or Formula (V^{A}-b′):

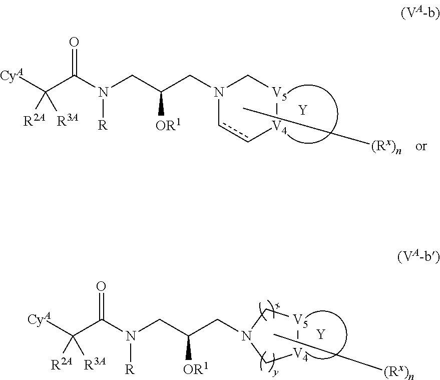

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (A), L_{z} is a linker L_{B} and Ring Z is a group Ar, to provide a compound of Formula (A-I^{B}):

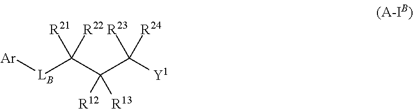

or a pharmaceutically acceptable salt thereof, wherein:

L_{B} is —N(R)C(O)—, —C(O)N(R)—, —N(R)C(O)N(R)—, —N(R)C(O)O—, or —OC(O)N(R)—;

each R is independently hydrogen or optionally substituted C₁₋₆ aliphatic;

Ar is a monocyclic or bicyclic aromatic ring having 0-4 heteroatoms independently selected from nitrogen, oxygen, and sulfur, wherein Ar is substituted with 0, 1, 2, 3, 4, or 5 R^{y} groups, as valency permits; or

Ar is a monocyclic or bicyclic heterocyclic ring having 1-4 heteroatoms independently selected from nitrogen, oxygen, and sulfur, wherein Ar is substituted with 0, 1, 2, 3, 4, or 5 R^{y} groups, as valency permits;

each R^{y} is independently selected from the group consisting of halo, —CN, —NO₂, optionally substituted aliphatic, optionally substituted carbocyclyl, optionally substituted aryl, optionally substituted heterocyclyl, optionally substituted heteroaryl, —OR^{A}, —N(R^{B})₂, —SR^{A}, —C(═O)R^{A}, —C(O)OR^{A}, —C(O)SR^{A}, —C(O)N(R^{B})₂, —C(O)N(R^{B})N(R^{B})₂, —OC(O)R^{A}, —OC(O)N(R^{B})₂, —NR^{B}C(O)R^{A}, —NR^{B}C(O)N(R^{B})₂, —NR^{B}C(O)N(R^{B})N(R^{B})₂, —NR^{B}C(O)OR^{A}, —SC(O)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(O)R^{A}, —OS(O)₂R^{A}, —SO₂R^{A}, —NR^{B}SO₂R^{A}, or —SO₂N(R^{B})₂;

each R^{A} is independently selected from the group consisting of hydrogen, optionally substituted aliphatic, optionally substituted carbocyclyl, optionally substituted heterocyclyl, optionally substituted aryl, and optionally substituted heteroaryl; and

each R^{B} is independently selected from the group consisting of hydrogen, optionally substituted aliphatic, optionally substituted carbocyclyl, optionally substituted heterocyclyl, optionally substituted aryl, and optionally substituted heteroaryl, or two R^{B} groups are taken together with their intervening atoms to form an optionally substituted heterocyclic ring.

In certain embodiments of Formula (A-I^{B}), wherein R¹² is hydrogen, and R¹³ is —OR¹, a provided compound is of Formula (I^{B}) or Formula (I^{B′}):

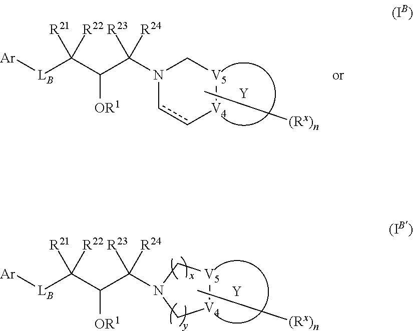

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (I^{B}) or Formula (I^{B′}), a provided compound is of Formula (I^{B}-a) or Formula (I^{B}-a′):

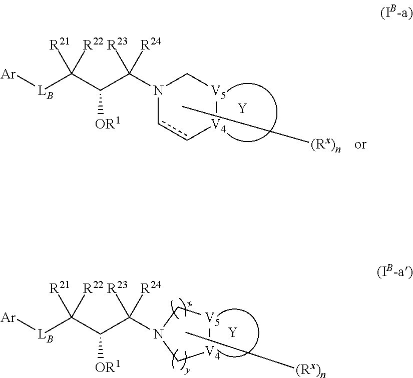

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (I^{B}) or Formula (I^{B′}), a provided compound is of Formula (I^{B}-b) or Formula (I^{B}-b′):

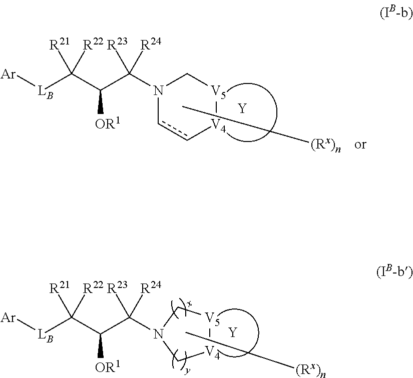

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (I^{B}) or Formula (I^{B′}), wherein R²¹-R²⁴ is hydrogen, a provided compound is of Formula (I^{B}-c) or Formula (I^{B}-c′):

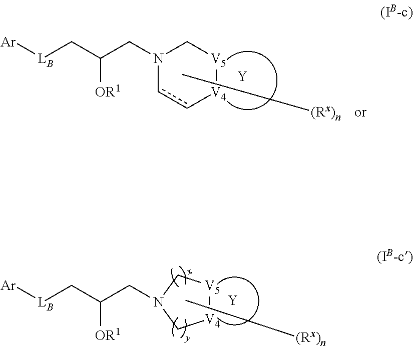

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (I^{B}) or Formula (I^{B′}), wherein R²¹-R²⁴ is hydrogen, a provided compound is of Formula (I^{B}-d) or Formula (I^{B}-d′):

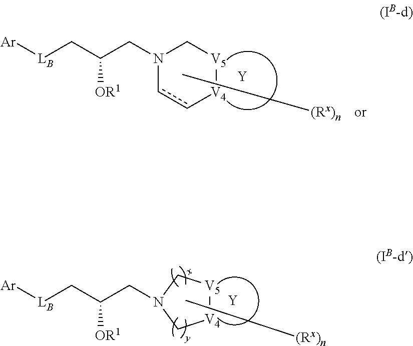

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (I^{B}) or Formula (I^{B′}), wherein R²¹-R²⁴ is hydrogen, a provided compound is of Formula (I^{B}-e) or Formula (I^{B}-e′):

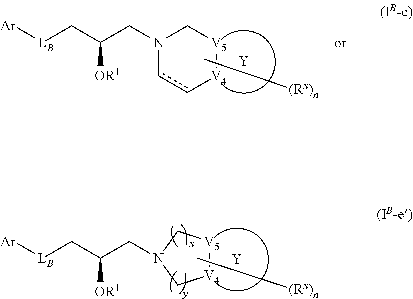

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (A-I^{B}), wherein L^{B} is —C(O)N(R)— and R²¹-R²⁴ is hydrogen, a provided compound is of Formula (II^{B}) or Formula (II^{B′}):

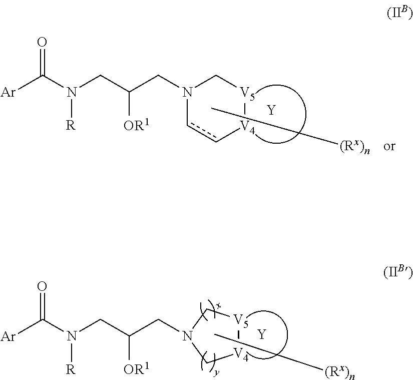

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (II^{B}) or Formula (II^{B′}), wherein L^{B} is —C(O)N(R)— and R²¹-R²⁴ is hydrogen, a provided compound is of Formula (II^{B}-a) or Formula (II^{B}-a′):

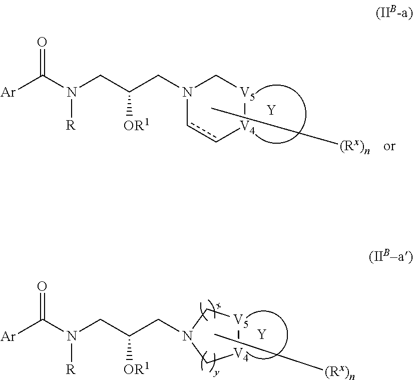

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (II^{B}) or Formula (II^{B′}), wherein L^{B} is —C(O)N(R)— and R²¹-R²⁴ is hydrogen, a provided compound is of Formula (II^{B}-b) or Formula (II^{B}-b′):

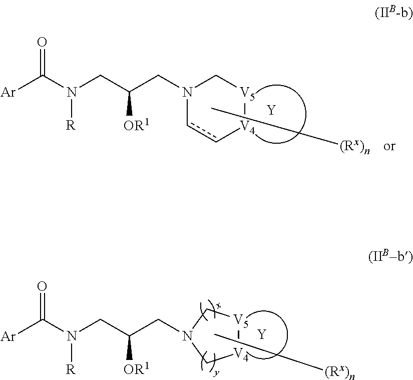

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (A-I^{B}), wherein Ar is optionally substituted phenyl, L^{B} is —C(O)N(R)— and R²¹-R²⁴ is hydrogen, a provided compound is of Formula (III^{B}) or Formula (III^{B′}):

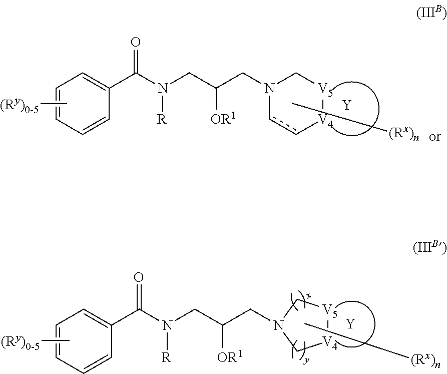

or a pharmaceutically acceptable salt thereof, comprising 0, 1, 2, 3, 4, or 5 R^{y} groups.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (III^{B}) or Formula (III^{B′}), wherein Ar is optionally substituted phenyl, L^{B} is —C(O)N(R)— and R²¹-R²⁴ is hydrogen, a provided compound is of Formula (III^{B}-a) or Formula (III^{B}-a′):

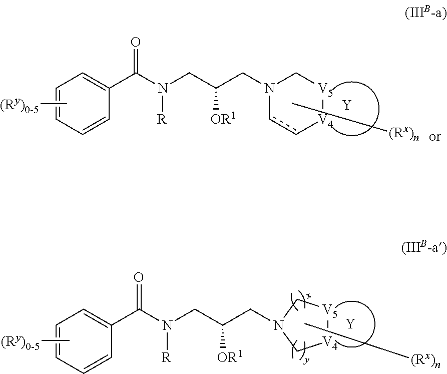

or a pharmaceutically acceptable salt thereof, comprising 0, 1, 2, 3, 4, or 5 R^{y} groups.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (III^{B}) or Formula (III^{B′}), wherein Ar is optionally substituted phenyl, L^{B} is —C(O)N(R)— and R²¹-R²⁴ is hydrogen, a provided compound is of Formula (III^{B}-b) or Formula (III^{B}-b′):

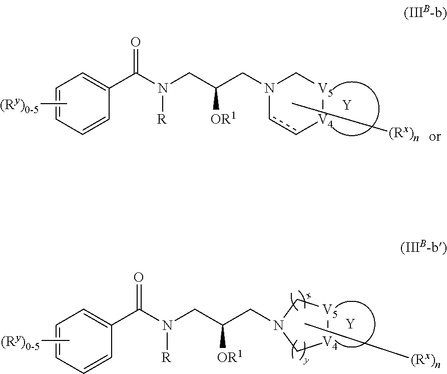

or a pharmaceutically acceptable salt thereof, comprising 0, 1, 2, 3, 4, or 5 R^{y} groups.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (A-I^{B}), wherein Ar is optionally substituted 2-, 3-, or 4-pyridinyl, L^{B} is —C(O)N(R)— and R²¹-R²⁴ is hydrogen, a provided compound is of Formula (IV^{B}), Formula (IV^{B′}), Formula (V^{B}), Formula (V^{B′}), Formula (VI^{B}), or Formula (VI^{B′}):

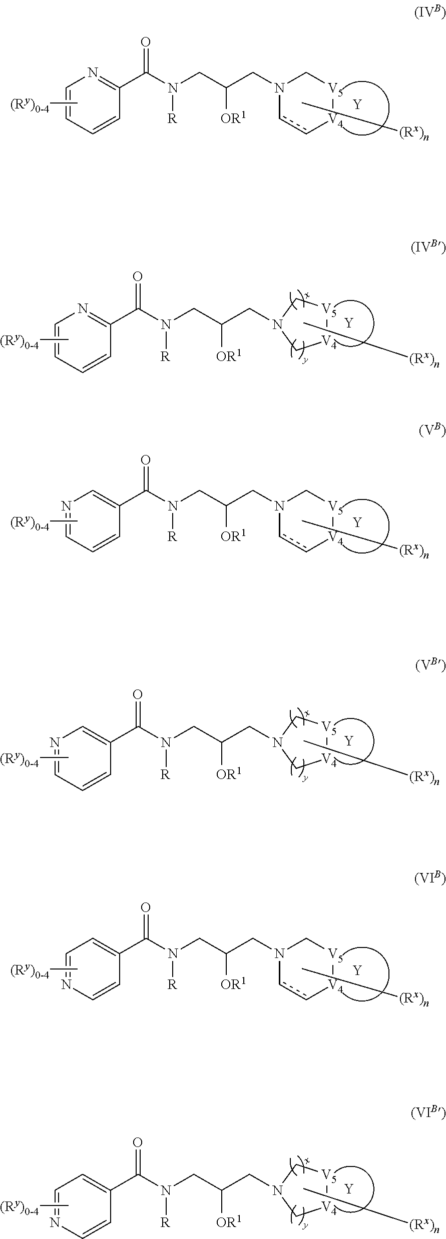

or a pharmaceutically acceptable salt thereof, comprising 0, 1, 2, 3, or 4 R^{y} groups.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (IV^{B}), Formula (IV^{B′}), Formula (V^{B}), Formula (V^{B′}), Formula (VI^{B}), or Formula (VI^{B′}), wherein Ar is optionally substituted 2-, 3-, or 4-pyridinyl, L^{B} is —C(O)N(R)— and R²¹-R²⁴ is hydrogen, a provided compound is of Formula Formula (IV^{B}-a), Formula (IV^{B′}-a), Formula (V^{B}-a), Formula (V^{B′}-a), Formula (VI^{B}-a), or Formula (VI^{B′}-a):

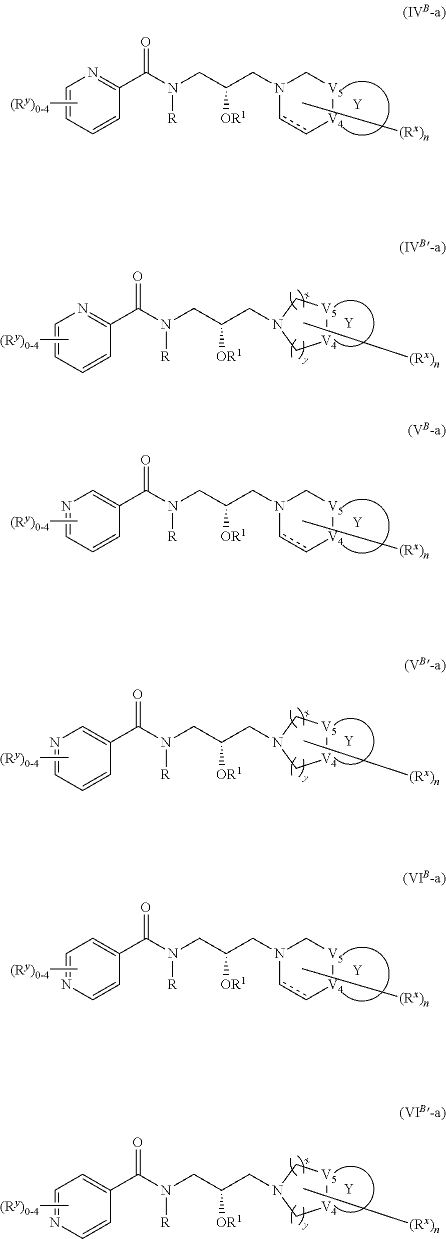

or a pharmaceutically acceptable salt thereof, comprising 0, 1, 2, 3, or 4 R^{y} groups.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (IV^{B}), Formula (IV^{B′}), Formula (V^{B}), Formula (V^{B′}), Formula (VI^{B}), or Formula (VI^{B′}), wherein Ar is optionally substituted 2-, 3-, or 4-pyridinyl, L^{B} is —C(O)N(R)— and R²¹-R²⁴ is hydrogen, a provided compound is of Formula Formula (IV^{B}-b), Formula (IV^{B′}-b), Formula (V^{B}-b), Formula (V^{B′}-b), Formula (VI^{B}-b), or Formula (VI^{B′}-b):

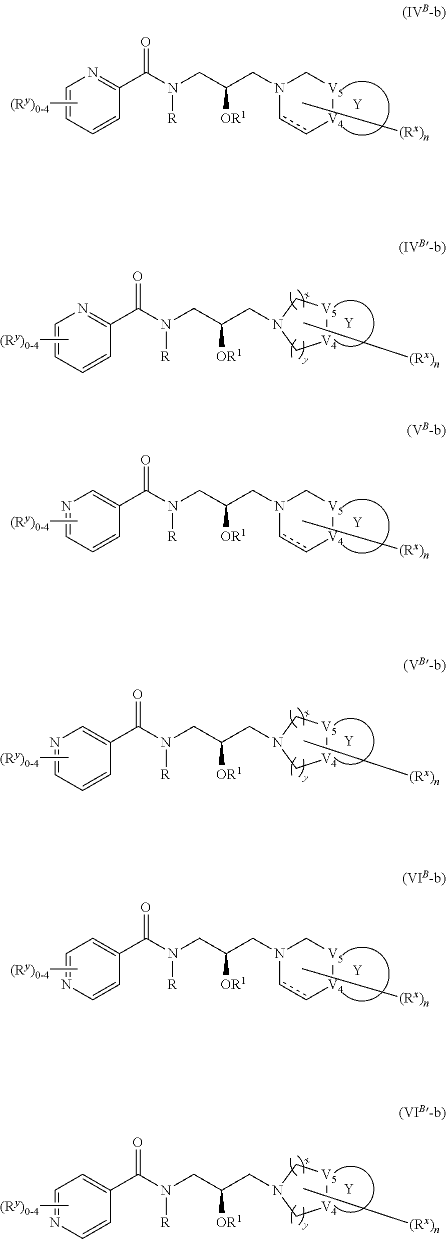

or a pharmaceutically acceptable salt thereof, comprising 0, 1, 2, 3, or 4 R^{y} groups.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (A-I^{B}), wherein Ar is optionally substituted pyridazinyl, L^{B} is —C(O)N(R)— and R²¹-R²⁴ is hydrogen, a provided compound is of Formula (VII^{B}), Formula (VII^{B}), Formula (VIII^{B}), or Formula (VIII^{B′}):

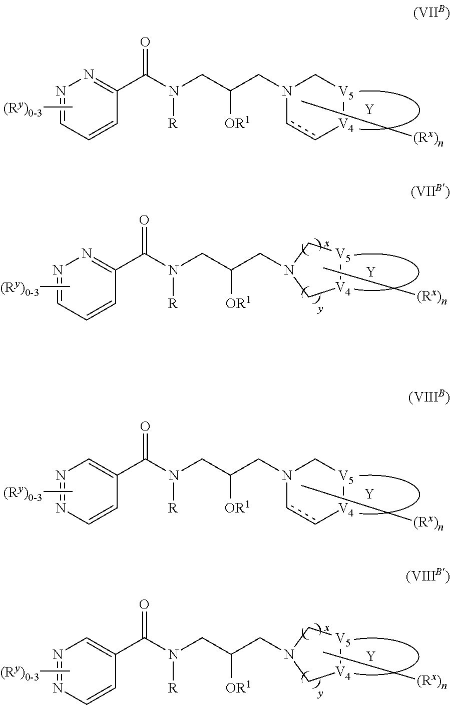

or a pharmaceutically acceptable salt thereof, comprising 0, 1, 2, or 3 R^{y} groups.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (VII^{B}), Formula (VII^{B′}), Formula (VIII^{B}), or Formula (VIII^{B′}), wherein Ar is optionally substituted pyridazinyl, L^{B} is —C(O)N(R)— and R²¹-R²⁴ is hydrogen, a provided compound is of Formula (VII^{B}-a), Formula (VII^{B′}-a), Formula (VIII^{B}-a), or Formula (VIII^{B′}-a):

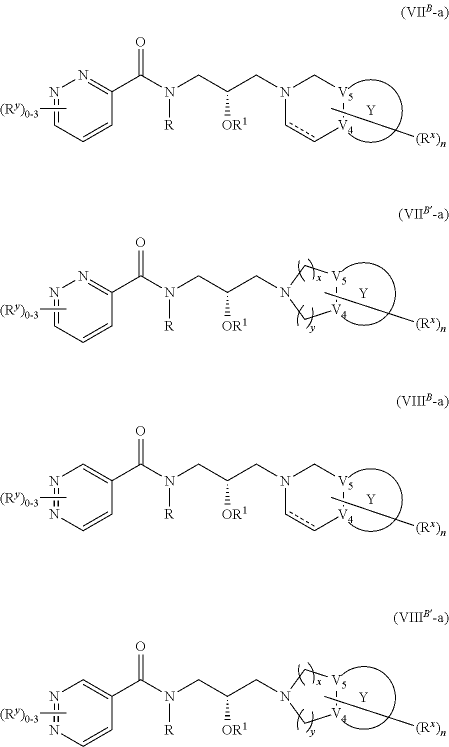

or a pharmaceutically acceptable salt thereof, comprising 0, 1, 2, or 3 R^{y} groups.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (VII^{B}), Formula (VII^{B′}), Formula (VIII^{B}), or Formula (VIII^{B′}), wherein Ar is optionally substituted pyridazinyl, L^{B} is —C(O)N(R)— and R²¹-R²⁴ is hydrogen, a provided compound is of Formula (VII^{B}-b), Formula (VII^{B′}-b), Formula (VIII^{B}-b), or Formula (VIII^{B′}-b):

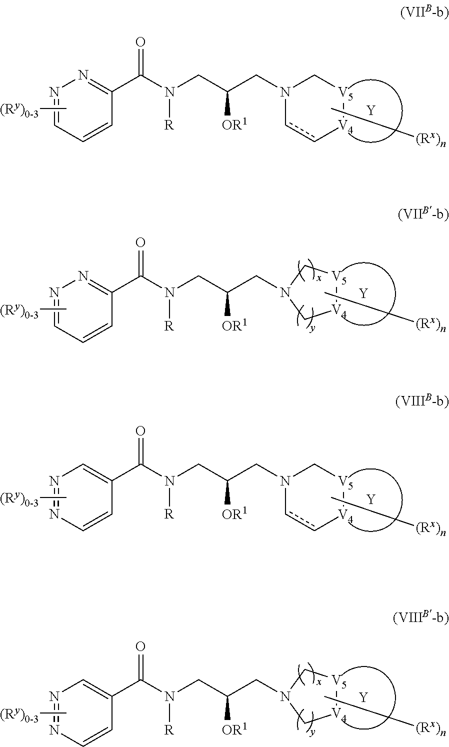

or a pharmaceutically acceptable salt thereof, comprising 0, 1, 2, or 3 R^{y} groups.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (A-I^{B}), wherein Ar is optionally substituted pyrazinyl, L^{B} is —C(O)N(R)— and R²¹-R²⁴ is hydrogen, a provided compound is of Formula (IX^{B}) or Formula (IX^{B′}):

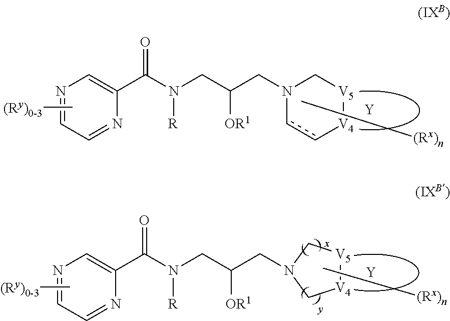

or a pharmaceutically acceptable salt thereof, comprising 0, 1, 2, or 3 R^{y} groups.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (A-I^{B}), wherein Ar is optionally substituted pyrazinyl, L^{B} is —C(O)N(R)— and R²¹-R²⁴ is hydrogen, a provided compound is of Formula (IX^{B}-a) or Formula (IX^{B′}-a):

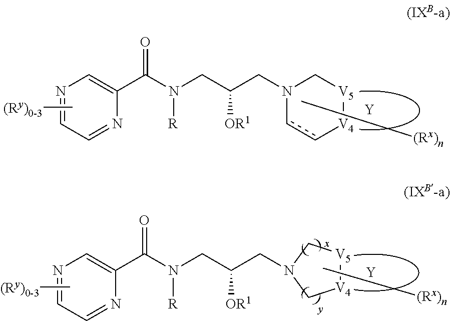

or a pharmaceutically acceptable salt thereof, comprising 0, 1, 2, or 3 R^{y} groups.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (A-I^{B}), wherein Ar is optionally substituted pyrazinyl, L^{B} is —C(O)N(R)— and R²¹-R²⁴ is hydrogen, a provided compound is of Formula (IX^{B}-b) or Formula (IX^{B′}-b):

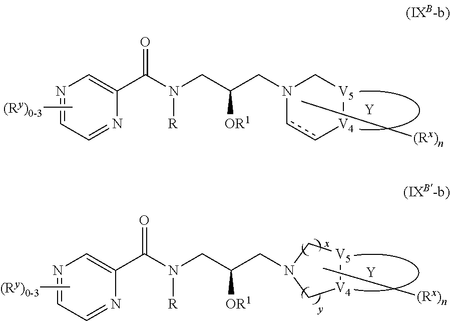

or a pharmaceutically acceptable salt thereof, comprising 0, 1, 2, or 3 R^{y} groups.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (A-I^{B}), wherein Ar is optionally substituted pyrimidinyl, L^{B} is —C(O)N(R)— and R²¹-R²⁴ is hydrogen, a provided compound is of Formula (X^{B}), Formula (X^{B′}), Formula (XI^{B}), Formula (XI^{B′}), Formula (XII^{B}), or Formula (XII^{B′}):

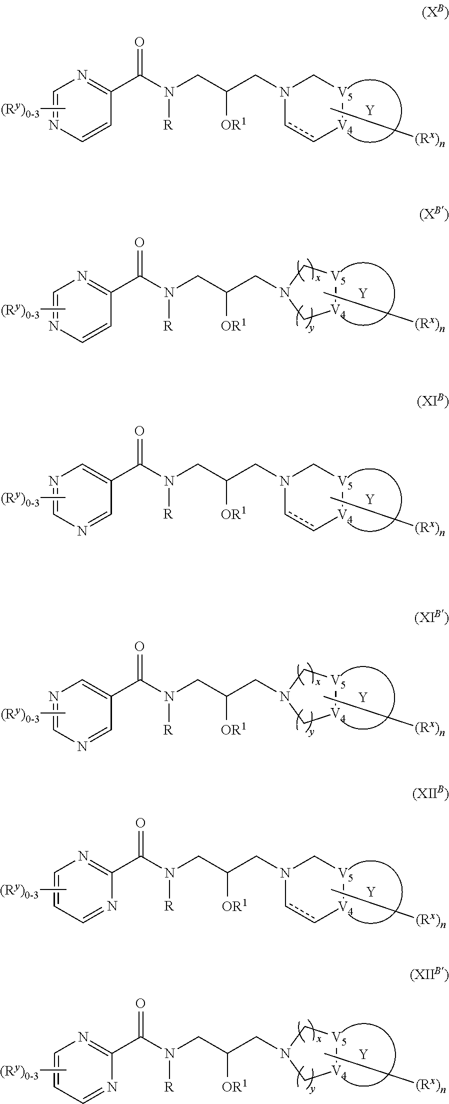

or a pharmaceutically acceptable salt thereof, comprising 0, 1, 2, or 3 R^{y} groups.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of (X^{B}), Formula (X^{B′}), Formula (XI^{B}), Formula (XI^{B′}), Formula (XII^{B}), or Formula (XII^{B′}), wherein Ar is optionally substituted pyrimidinyl, L^{B} is —C(O)N(R)— and R²¹-R²⁴ is hydrogen, a provided compound is of Formula (X^{B}-a), Formula (X^{B′}-a), Formula (XI^{B}-a), Formula (XI^{B′}-a), Formula (XII^{B}-a), or Formula (XII^{B′}-a):

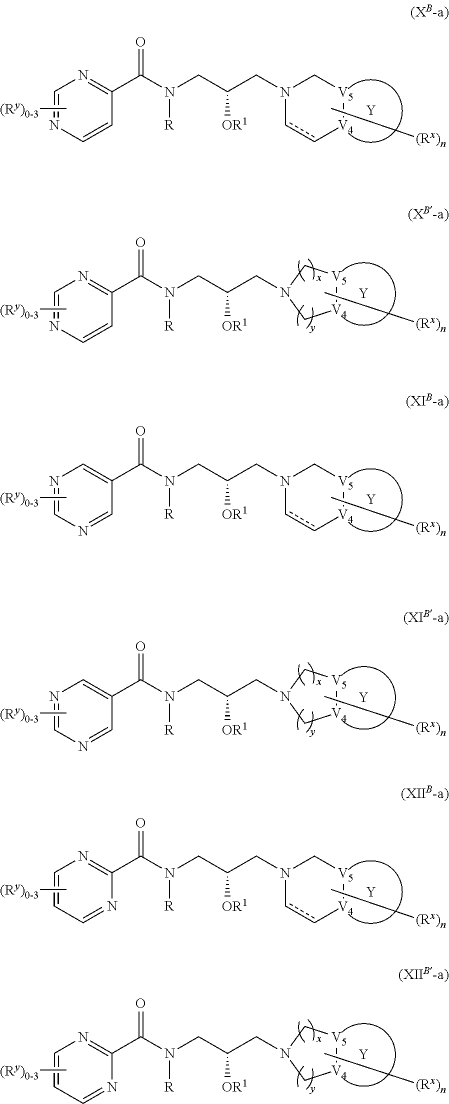

or a pharmaceutically acceptable salt thereof, comprising 0, 1, 2, or 3 R^{y} groups.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of (X^{B}), Formula (X^{B′}), Formula (XI^{B}), Formula (XI^{B′}), Formula (XII^{B}), or Formula (XII^{B′}), wherein Ar is optionally substituted pyrimidinyl, L^{B} is —C(O)N(R)— and R²¹-R²⁴ is hydrogen, a provided compound is of Formula (X^{B}-b), Formula (X^{B′}-b), Formula (XI^{B}-b), Formula (XI^{B′}-b), Formula (XII^{B}-b), or Formula (XII^{B′}-b):

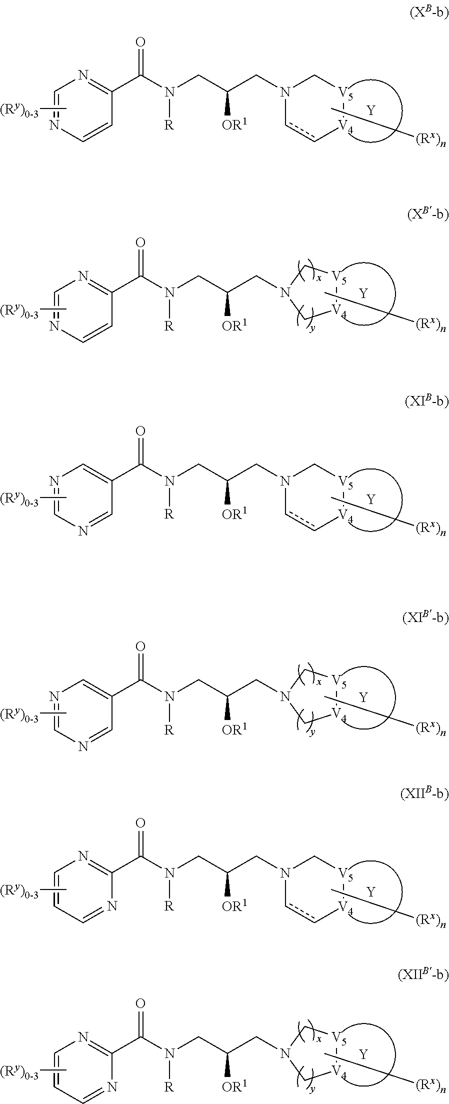

or a pharmaceutically acceptable salt thereof, comprising 0, 1, 2, or 3 R^{y} groups.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (A), wherein L_{z} is absent, and Ring Z is a group of formula (also referred to herein as Ring C):

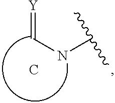

the present disclosure provides a compound of Formula (A-I^{C}):

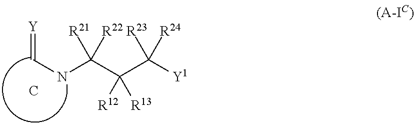

or a pharmaceutically acceptable salt thereof, wherein Ring C is an optionally substituted, 5- to 12-membered, monocyclic or bicyclic, heterocyclyl or heteroaryl having 1-4 heteroatoms independently selected from nitrogen, oxygen, and sulfur; and Y is O or S.

In certain embodiments of Formula (A-I^{C}), wherein R¹² is hydrogen, and R¹³ is —OR¹, a provided compound is of Formula (I^{C}) or Formula (I^{C′}):

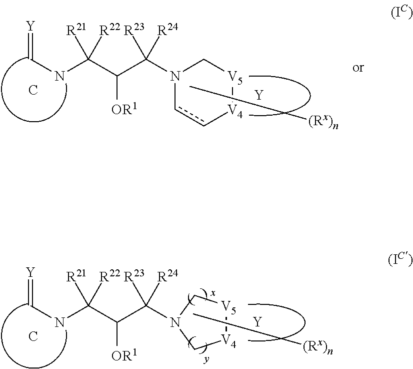

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (I^{C}) or Formula (I^{C′}), a provided compound is of Formula (I^{C}-a) or Formula (I^{C}-a′):

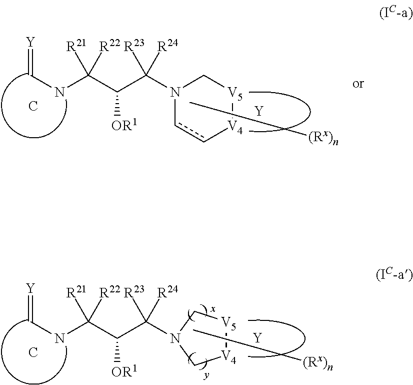

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (I^{C}) or Formula (I^{C′}), a provided compound is of Formula (I^{C}-b) or Formula (I^{C}-b′):

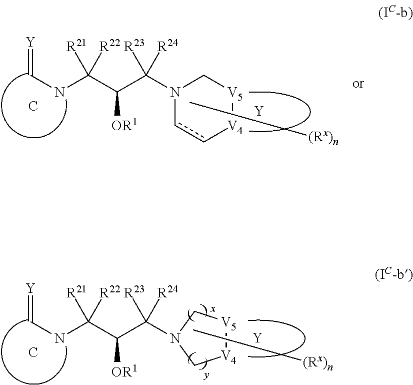

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (I^{C}) or Formula (I^{C′}), wherein R²¹-R²⁴ is hydrogen, a provided compound is of Formula (I^{C}-c) or Formula (I^{C}-c′):

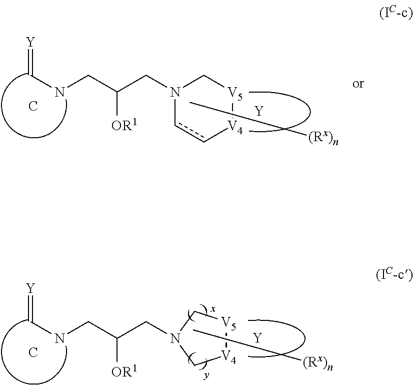

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (I^{C}) or Formula (I^{C′}), wherein R²¹-R²⁴ is hydrogen, a provided compound is of Formula (I^{C}-d) or Formula (I^{C}-d′):

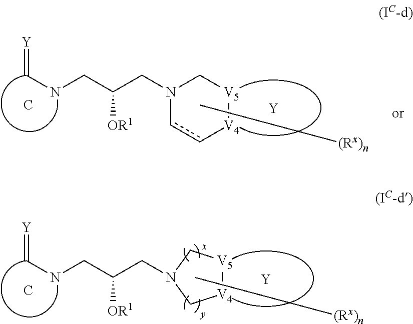

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (I^{C}) or Formula (I^{C′}), wherein R²¹-R²⁴ is hydrogen, a provided compound is of Formula (I^{C}-e) or Formula (I^{C}-e′):

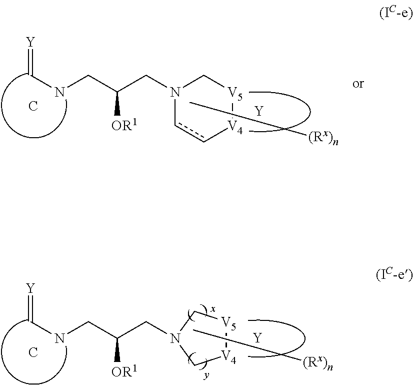

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (A-I^{C}), wherein R²¹-R²⁴ is hydrogen and wherein Ring C is a group of formula:

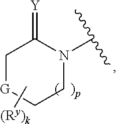

a provided compound is of Formula (A-II^{C}):

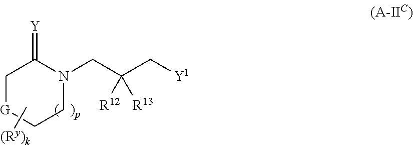

or a pharmaceutically acceptable salt thereof,
wherein:

G is NR^{2C}, CR^{3C}R^{4C}, O or S;

R^{2C} is selected from the group consisting of optionally substituted aliphatic, optionally substituted carbocyclyl, optionally substituted aryl, optionally substituted heterocyclyl, optionally substituted heteroaryl, —C(O)R^{A}, —C(O)OR^{A}, —C(O)SR^{A}, —C(O)N(R^{B})₂, —C(═NR^{B})R^{A}, —C(═NR^{B})N(R^{B})₂, —C(═S)R^{A}, —C(═S)N(R^{B})₂, —S(═O)R^{A}, —SO₂R^{A}, and —SO₂N(R^{B})₂;

R^{3C} is selected from the group consisting of hydrogen, halo, optionally substituted aliphatic, optionally substituted carbocyclyl, optionally substituted aryl, optionally substituted heterocyclyl, optionally substituted heteroaryl, —OR^{A}, —N(R^{B})₂, —SR^{A}, —C(═O)R^{A}, —C(O)OR^{A}, —C(O)SR^{A}, —C(O)N(R^{B})₂, —C(O)N(R^{B})N(R^{B})₂, —OC(O)R^{A}, —OC(O)N(R^{B})₂, —NR^{B}C(O)R^{A}, —NR^{B}C(O)N(R^{B})₂, —NR^{B}C(O)N(R^{B})N(R^{B})₂, —NR^{B}C(O)OR^{A}, —SC(O)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(O)R^{A}, —OS(O)₂R^{A}, —SO₂R^{A}, —NR^{B}SO₂R^{A}, or —SO₂N(R^{B})₂;

each R^{A} is independently selected from the group consisting of hydrogen, optionally substituted aliphatic, optionally substituted carbocyclyl, optionally substituted heterocyclyl, optionally substituted aryl, and optionally substituted heteroaryl;

each R^{B} is independently selected from the group consisting of hydrogen, optionally substituted aliphatic, optionally substituted carbocyclyl, optionally substituted heterocyclyl, optionally substituted aryl, and optionally substituted heteroaryl, or two R^{B} groups are taken together with their intervening atoms to form an optionally substituted heterocyclic ring;

R^{4C} is selected from the group consisting of hydrogen, halo, or optionally substituted aliphatic;

each R^{y} is independently selected from the group consisting of halo, —CN, —NO₂, optionally substituted aliphatic, optionally substituted carbocyclyl, optionally substituted aryl, optionally substituted heterocyclyl, optionally substituted heteroaryl, —OR^{A}, —N(R^{B})₂, —SR^{A}, —C(═O)R^{A}, —C(O)OR^{A}, —C(O)SR^{A}, —C(O)N(R^{B})₂, —C(O)N(R^{B})N(R^{B})₂, —OC(O)R^{A}, —OC(O)N(R^{B})₂, —NR^{B}C(O)R^{A}, —NR^{B}C(O)N(R^{B})₂, —NR^{B}C(O)N(R^{B})N(R^{B})₂, —NR^{B}C(O)OR^{A}, —SC(O)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(O)R^{A}, —OS(O)₂R^{A}, —SO₂R^{A}, —NR^{B}SO₂R^{A}, or —SO₂N(R^{B})₂,

or two adjacent R^{y} groups may be taken together with their intervening atoms to form a saturated, partially unsaturated, or aromatic ring having 0-4 heteroatoms independently selected from nitrogen, oxygen, and sulfur;

p is 0, 1, or 2; and

k is 0, 1, 2, 3, or 4.

In certain embodiments of Formula (A-II^{C}), wherein R¹² is hydrogen and R¹³ is —OR¹, a provided compound is of Formula (II^{C}) or Formula (II^{C′}):

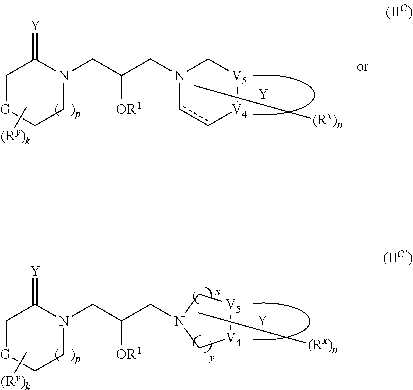

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (II^{C}) or Formula (II^{C′}), a provided compound is of Formula (II^{C}-a) or Formula (II^{C}-a′):

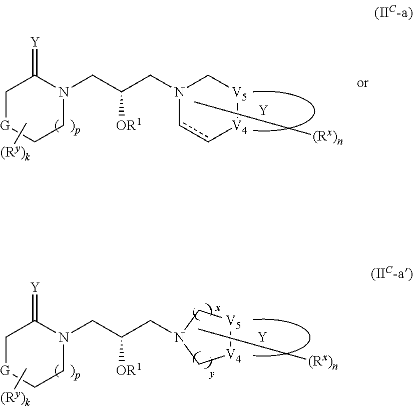

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (II^{C}) or Formula (II^{C}), a provided compound is of Formula (II^{C}-b) or Formula (II^{C}-b′):

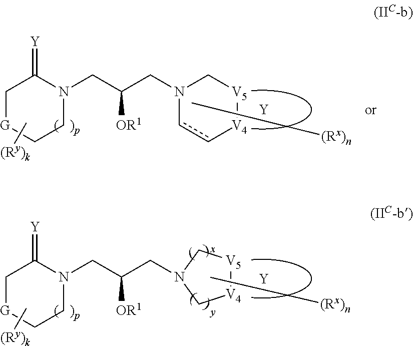

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (A-II^{C}), wherein G is NR^{2C}, a provided compound is of Formula (III^{C}) or Formula (III^{C′}):

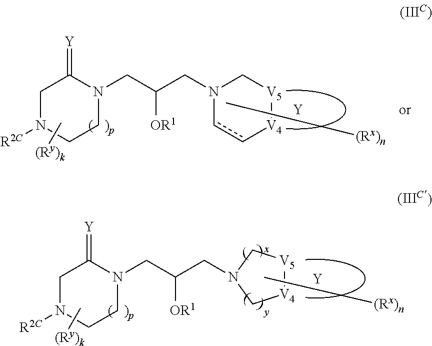

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (III^{C}) or Formula (III^{C}), wherein G is NR^{2C}, a provided compound is of Formula (III^{C}-a) or Formula (III^{C}-a′):

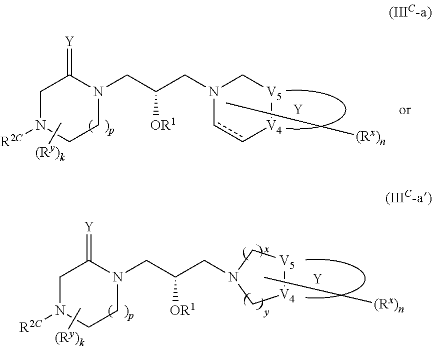

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (III^{C}) or Formula (III^{C′}), wherein G is NR^{2C}, a provided compound is of Formula (III^{C}-b) or Formula (III^{C}-b′):

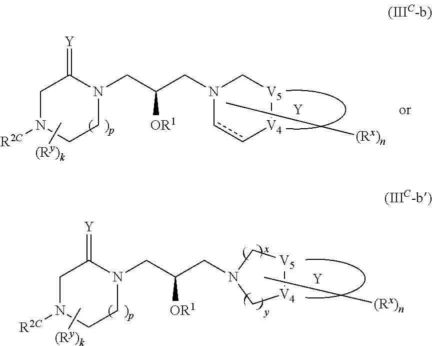

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (A-II^{C}), wherein Y is O, G is CR³CR^{4C}, and R^{4C} is hydrogen, a provided compound is of Formula (IV^{C}) or Formula (IV^{C′}):

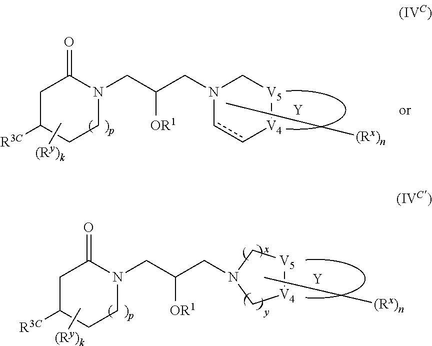

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (IV^{C}) or Formula (IV^{C′}), wherein Y is O, G is CR^{3C}R^{4C}, and R^{4C} is hydrogen, a provided compound is of Formula (IV^{C}-a) or Formula (IV^{C}-a′):

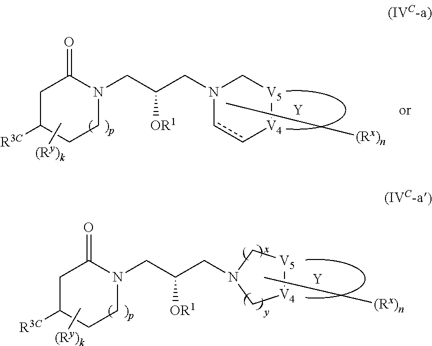

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (IV^{C}) or Formula (IV^{C′}), a provided compound is of Formula (IV^{C}-b) or Formula (IV^{C}-b′):

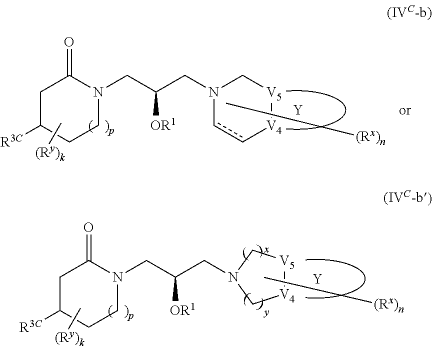

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (A-I^{C}), wherein R²¹-R²⁴ is hydrogen and wherein Ring C is a group of formula:

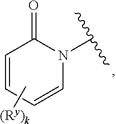

a provided compound is of Formula (V^{C}) or Formula (V^{C′}):

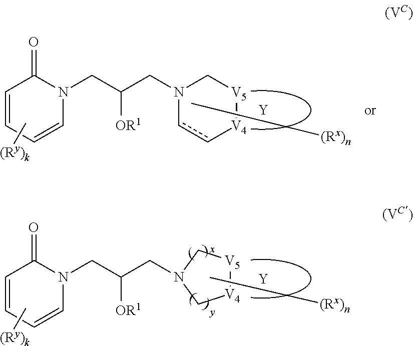

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{C}) or Formula (V^{C′}), a provided compound is of Formula (V^{C}-a) or Formula (V^{C}-a′):

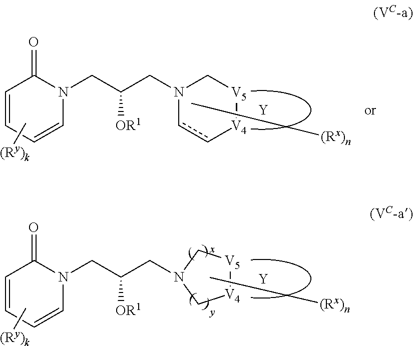

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{C}) or Formula (V^{C′}), a provided compound is of Formula (V^{C}-b) or Formula (V^{C}-b′):

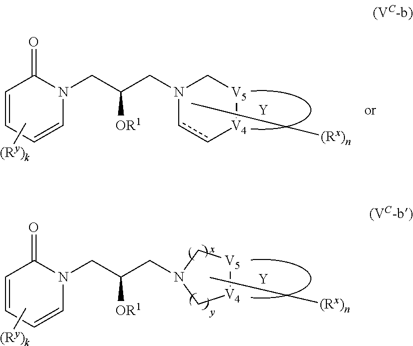

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (A-I^{C}), wherein R²¹-R²⁴ is hydrogen and wherein Ring C is a group of formula:

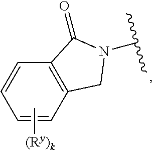

a provided compound is of Formula (VI^{C}) or Formula (VI^{C′}):

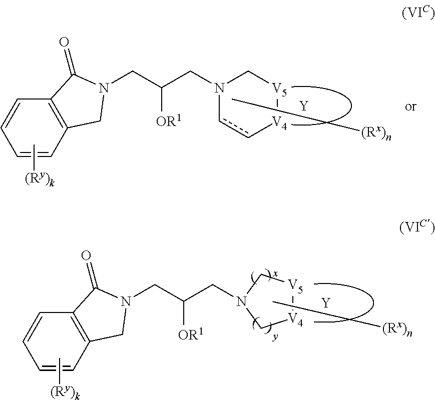

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (VI^{C}) or Formula (VI^{C′}), a provided compound is of Formula (VI^{C}-a) or Formula (VI^{C}-a′):

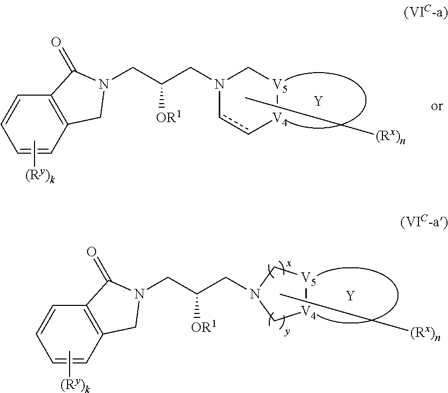

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (VI^{C}) or Formula (VI^{C′}), a provided compound is of Formula (VI^{C}-b) or Formula (VI^{C}-b′):

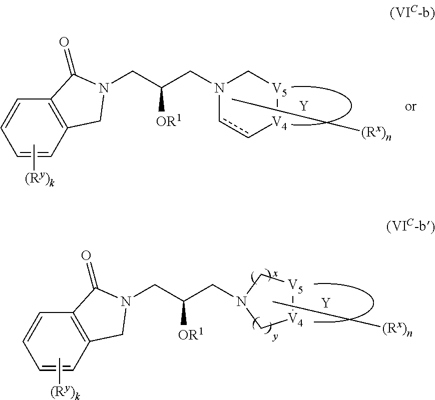

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (A), wherein L_{z} is L_{D}, and Ring Z is a group of formula (also referred to herein as Ring A):

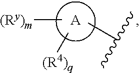

the present disclosure provides a compound of Formula (A-I^{D}):

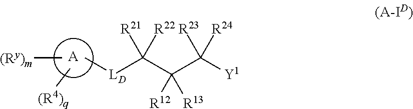

or a pharmaceutically acceptable salt thereof,
wherein:

L_{D} is the linker L_{B} wherein L_{B} is —N(R)C(O)—, —C(O)N(R)—, —N(R)C(O)N(R)—, —N(R)C(O)O—, or —OC(O)N(R)— and each R is independently hydrogen or optionally substituted C₁₋₆ aliphatic;

or

L_{D} is a linker selected from the group consisting of —O—, —N(R)—, —C(R^{2A})(R^{3A})—, —O—CR^{2A}R^{3A}, —N(R)—CR^{2A}R^{3A}—, —O—CR^{2A}R^{3A}—O—, —N(R)—CR^{2A}R^{3A}—O, —N(R)—CR^{2A}R^{3A}—N(R)—, —O—CR^{2A}R^{3A}, —N(R)—, —CR^{2A}R^{3A}—O—, —CR^{2A}R^{3A}—N(R)—, —O—CR^{2A}R^{3A}—CR⁹R¹⁰—, —N(R)—CR^{2A}R^{3A}—CR⁹R¹⁰—, —CR^{2A}R^{3A}—CR⁹R¹⁰—O—, —CR^{2A}R^{3A}—CR⁹R¹⁰—N(R)—, or —CR^{2A}R^{3A}—CR⁹R¹⁰—;

each R is independently hydrogen or optionally substituted C₁₋₆ aliphatic;

R^{2A} and R^{3A} are independently selected from the group consisting of hydrogen, halo, —CN, —NO₂, optionally substituted aliphatic, optionally substituted carbocyclyl; optionally substituted phenyl, optionally substituted heterocyclyl, optionally substituted heteroaryl, —OR^{A}, —N(R^{B})₂, —SR^{A}, —C(═O)R^{A}, —C(O)OR^{A}, —C(O)SR^{A}, —C(O)N(R^{B})₂, —C(O)N(R^{B})N(R^{B})₂, —OC(O)R^{A}, —OC(O)N(R^{B})₂, —NR^{B}C(O)R^{A}, —NR^{B}C(O)N(R^{B})₂, —NR^{B}C(O)N(R^{B})N(R^{B})₂, —NR^{B}C(O)OR^{A}, —SC(O)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(O)R^{A}, —OS(O)₂R^{A}, —SO₂R^{A}, —NR^{B}SO₂R^{A}, or —SO₂N(R^{B})₂; or R^{2A} and R^{3A} are taken together with their intervening atoms to form an optionally substituted carbocyclic or heterocyclic ring;

each R^{A} is independently selected from the group consisting of hydrogen, optionally substituted aliphatic, optionally substituted carbocyclyl, optionally substituted heterocyclyl, optionally substituted aryl, and optionally substituted heteroaryl;

each R^{B} is independently selected from the group consisting of hydrogen, optionally substituted aliphatic, optionally substituted carbocyclyl, optionally substituted heterocyclyl, optionally substituted aryl, and optionally substituted heteroaryl, or two R^{B} groups are taken together with their intervening atoms to form an optionally substituted heterocyclic ring;

Ring A is a monocyclic or bicyclic, saturated, partially unsaturated, or aromatic ring having 0-4 heteroatoms independently selected from nitrogen, oxygen, and sulfur;

R⁴ is -L₁-Cy^{D};

L₁ is a bond, —O—, —S—, —N(R)—, —C(O)—, —C(O)N(R)—, —N(R)C(O)N(R)—, —N(R)C(O)—, —N(R)C(O)O—, —OC(O)N(R)—, —SO₂—, —SO₂N(R)—, —N(R)SO₂—, —OC(O)—, —C(O)O—, or an optionally substituted, straight or branched, C₁₋₆ aliphatic chain wherein one, two, or three methylene units of L₁ are optionally and independently replaced by —O—, —S—, —N(R)—, —C(O)—, —C(O)N(R)—, —N(R)C(O)N(R)—, —N(R)C(O)—, —N(R)C(O)O—, —OC(O)N(R)—, —SO₂—, —SO₂N(R)—, —N(R)SO₂—, —OC(O)—, or —C(O)O—;

Cy^{D} is an optionally substituted, monocyclic, bicyclic or tricyclic, saturated, partially unsaturated, or aromatic ring having 0-4 heteroatoms independently selected from nitrogen, oxygen, and sulfur;

R⁹ and R¹⁰ are independently selected from the group consisting of hydrogen, halo, —CN, —NO₂, optionally substituted aliphatic, optionally substituted carbocyclyl; optionally substituted phenyl, optionally substituted heterocyclyl, optionally substituted heteroaryl, —OR^{A}, —N(R^{B})₂, —SR^{A}, —C(═O)R^{A}, —C(O)OR^{A}, —C(O)SR^{A}, —C(O)N(R^{B})₂, —C(O)N(R^{B})N(R^{B})₂, —OC(O)R^{A}, —OC(O)N(R^{B})₂, —NR^{B}C(O)R^{A}, —NR^{B}C(O)N(R^{B})₂, —NR^{B}C(O)N(R^{B})N(R^{B})₂, —NR^{B}C(O)OR^{A}, —SC(O)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(O)R^{A}, —OS(O)₂R^{A}, —SO₂R^{A}, —NR^{B}SO₂R^{A}, or —SO₂N(R^{B})₂; or R⁹ and R¹⁰ are taken together with their intervening atoms to form an optionally substituted carbocyclic or heterocyclic ring;

each R^{y} is independently selected from the group consisting of halo, —CN, —NO₂, optionally substituted aliphatic, optionally substituted carbocyclyl; optionally substituted aryl, optionally substituted heterocyclyl, optionally substituted heteroaryl, —OR^{A}, —N(R^{B})₂, —SR^{A}, —C(═O)R^{A}, —C(O)OR^{A}, —C(O)SR^{A}, —C(O)N(R^{B})₂, —C(O)N(R^{B})N(R^{B})₂, —OC(O)R^{A}, —OC(O)N(R^{B})₂, —NR^{B}C(O)R^{A}, —NR^{B}C(O)N(R^{B})₂, —NR^{B}C(O)N(R^{B})N(R^{B})₂, —NR^{B}C(O)OR^{A}, —SC(O)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(O)R^{A}, —OS(O)₂R^{A}, —SO₂R^{A}, —NR^{B}SO₂R^{A}, or —SO₂N(R^{B})₂;

m is 0, 1, 2, 3, 4, 5, 6, 7, or 8, as valency permits; and

q is 0 or 1.

In certain embodiments of Formula (A^{D}), wherein R¹² is hydrogen, and R¹³ is —OR¹, a provided compound is of Formula (I^{D}) or Formula (I^{D′}):

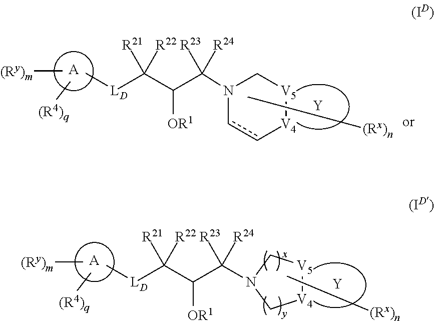

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (I^{D}) or Formula (I^{D′}), a provided compound is of Formula (I^{D}-a) or Formula (I^{D}-a′):

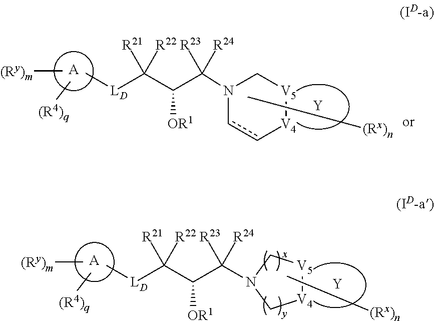

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (I^{D}) or Formula (I^{D′}), a provided compound is of Formula (I^{D}-b) Formula (I^{D}-b′):

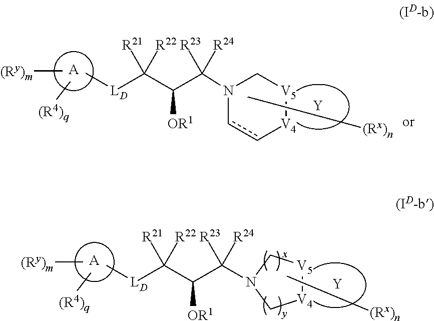

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (I^{D}) or Formula (I^{D′}), wherein R²¹-R²⁴ is hydrogen, a provided compound is of Formula (I^{D}-c) or Formula (I^{D}-c′):

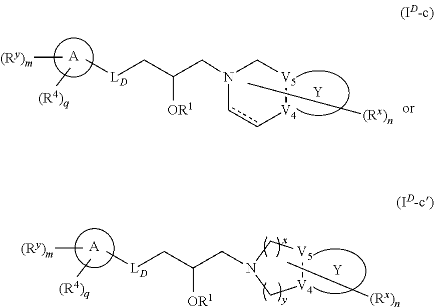

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (I^{D}) or Formula (I^{D′}), wherein R²¹-R²⁴ is hydrogen, a provided compound is of Formula (I^{D}-d) or Formula (I^{D}-d′):

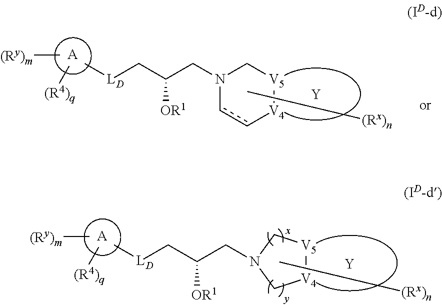

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (I^{D}) or Formula (I^{D′}), wherein R²¹-R²⁴ is hydrogen, a provided compound is of Formula (I^{D}-e) or Formula (I^{D}-e′):

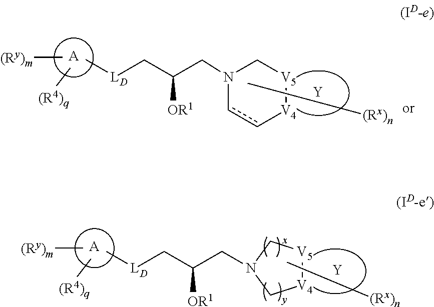

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (I^{D}) or Formula (I^{D′}), wherein L_{D} is —NR—, a provided compound is of Formula (II^{D}) or Formula (II^{D′}):

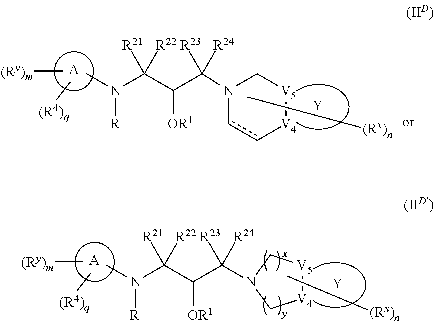

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (I^{D}) or Formula (I^{D′}), wherein L_{D} is —NR—, a provided compound is of Formula (II^{D}-a) or Formula (II^{D}-a′):

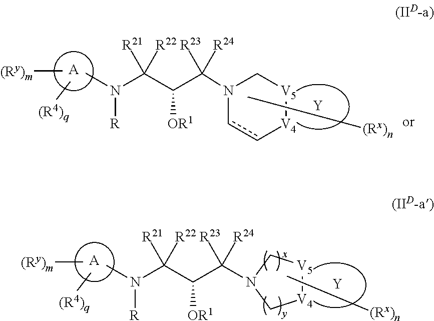

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (I^{D}) or Formula (I^{D′}), wherein L_{D} is —NR—, a provided compound is of Formula (II^{D}-b) or Formula (II^{D}-b′):

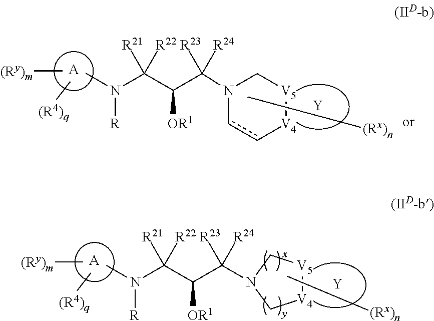

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (I^{D}) or Formula (I^{D′}), wherein L_{D} is —NR— and R²¹-R²⁴ is hydrogen, a provided compound is of Formula (II^{D}-c) or Formula (II^{D}-c′):

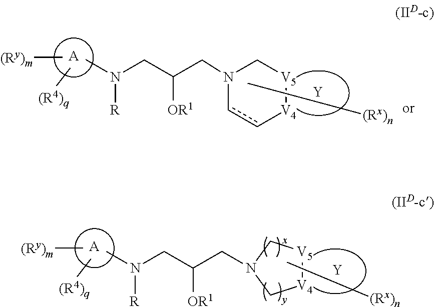

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (I^{D}) or Formula (I^{D′}), wherein L_{D} is —NR— and R²¹-R²⁴ is hydrogen, a provided compound is of Formula (II^{D}-d) or Formula (II^{D}-d′):

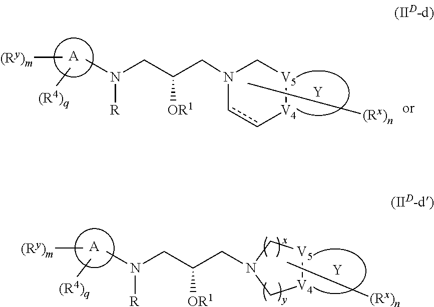

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (I^{D}) or Formula (I^{D′}), wherein L_{D} is —NR— and R²¹-R²⁴ is hydrogen, a provided compound is of Formula (II^{D}-e) or Formula (II^{D}-e′):

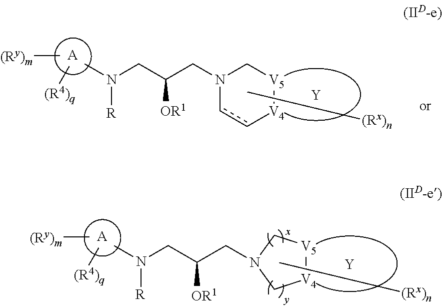

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (I^{D}) or Formula (I^{D′}), wherein L_{D} is —C(R^{2A})(R^{3A})—, a provided compound is of Formula (III^{D}) or Formula (III^{D′}):

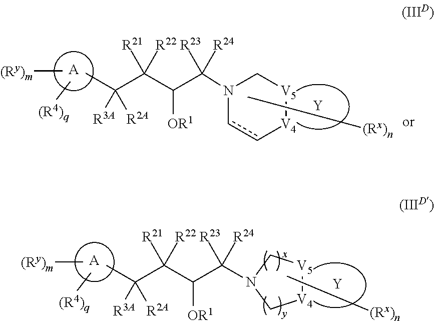

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (I^{D}) or Formula (I^{D′}), wherein L_{D} is —C(R^{2A})(R^{3A})—, provided compound is of Formula (III^{D}-a) or Formula (III^{D}-a′):

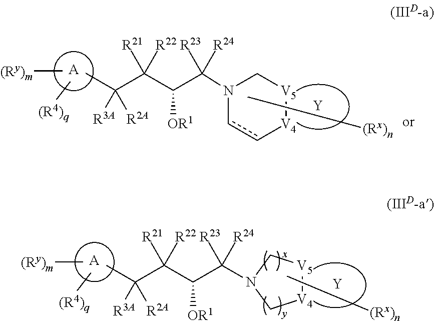

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (I^{D}) or Formula (I^{D′}), wherein L_{D} is —C(R^{2A})(R^{3A})—, a provided compound is of Formula (III^{D}-b) or Formula (III^{D}-b′):

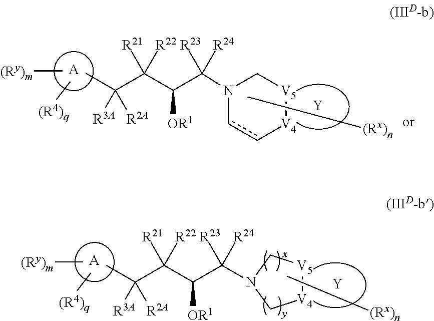

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (I^{D}) or Formula (I^{D′}), wherein L_{D} is —C(R^{2A})(R^{3A})— and R²¹-R²⁴ is hydrogen, a provided compound is of Formula (III^{D}-c) or Formula (III^{D}-c′):

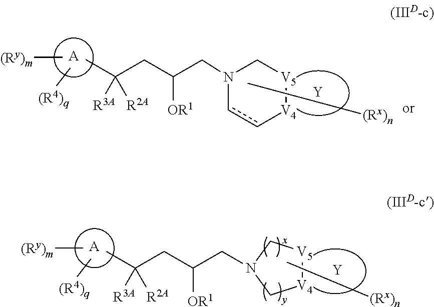

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (I^{D}) or Formula (I^{D′}), wherein L_{D} is —C(R^{2A})(R^{3A})— and R²¹-R²⁴ is hydrogen, a provided compound is of Formula (III^{D}-d) or Formula (III^{D}-d′):

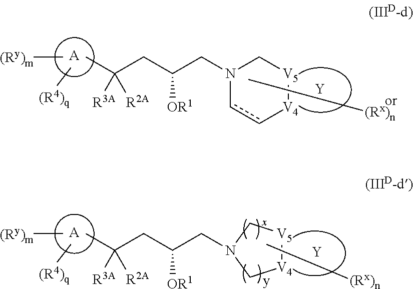

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (I^{D}) or Formula (I^{D′}), wherein L_{D} is —C(R^{2A})(R^{3A})— and R²¹-R²⁴ is hydrogen, a provided compound is of Formula (III^{D}-e) or Formula (III^{D}-e′):

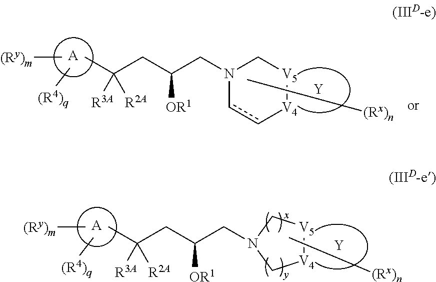

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (I^{D}) or Formula (I^{D′}), wherein L_{D} is —O—, a provided compound is of Formula (IV^{D}) or Formula (IV^{D′})

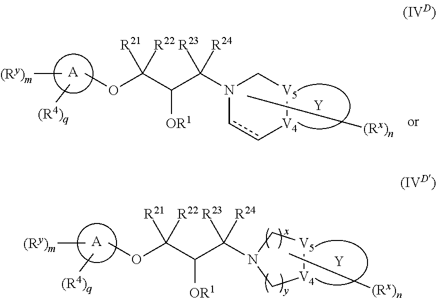

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (I^{D}) or Formula (I^{D′}), wherein L_{D} is —O—, a provided compound is of Formula (IV^{D}-a) or Formula (IV^{D}-a′):

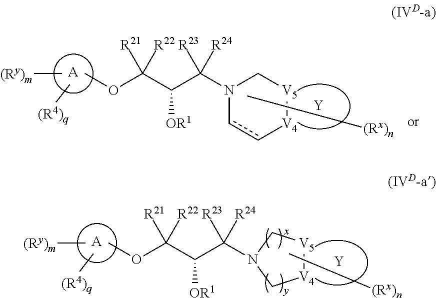

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (I^{D}) or Formula (I^{D′}), wherein L_{D} is —O—, a provided compound is of Formula (IV^{D}-b) or Formula (IV^{D}-b′):

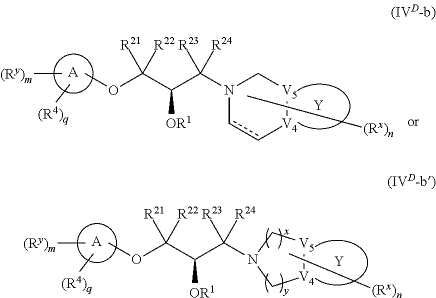

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (I^{D}) or Formula (I^{D′}), wherein L_{D} is —O— and R²¹-R²⁴ is hydrogen, a provided compound is of Formula (IV^{D}-c) or Formula (IV^{D}-c′):

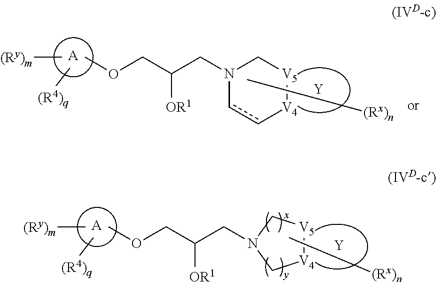

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (I^{D}) or Formula (I^{D′}), wherein L_{D} is —O— and R²¹-R²⁴ is hydrogen, a provided compound is of Formula (IV^{D}-d) or Formula (IV^{D}-d′):

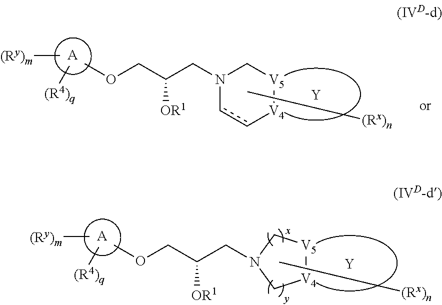

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (I^{D}) or Formula (I^{D′}), wherein L_{D} is —O— and R²¹-R²⁴ is hydrogen, a provided compound is of Formula (IV^{D}-e) or Formula (IV^{D}-e′):

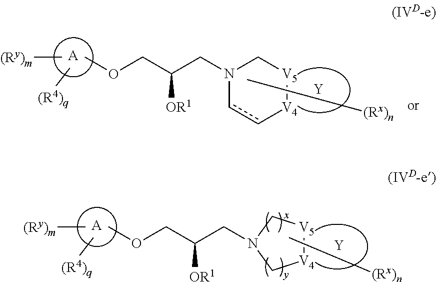

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (I^{D}) or Formula (I^{D′}), wherein L_{D} is —O— and R²¹-R²⁴ is hydrogen, a provided compound is of Formula (IV^{D}-e) or Formula (IV^{D}-e′):

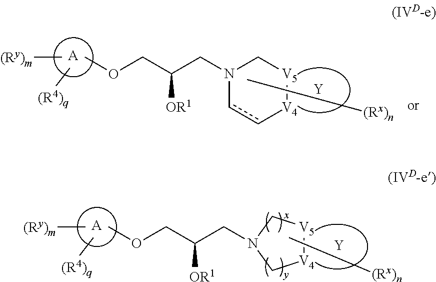

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (I^{D}) or Formula (I^{D′}), wherein L_{D} is linker L_{B}, and L_{B} is —C(O)NR—, a provided compound is of Formula (XX^{D}) or Formula (XX^{D′}):

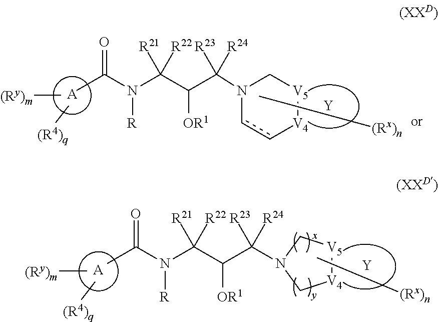

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (XX^{D}) or Formula (XX^{D′}), wherein L_{D} is linker L_{B}, and L_{B} is —C(O)NR—, a provided compound is of Formula (XX^{D}-a) or Formula (XX^{D}-a′):

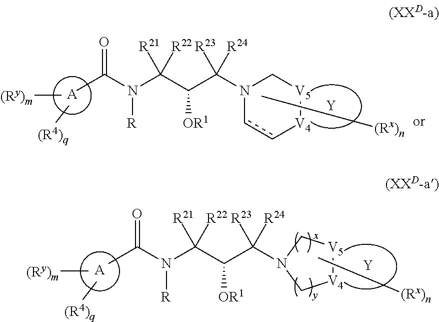

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (I^{D}) or Formula (I^{D′}), wherein L_{D} is linker L_{B}, and L_{B} is —C(O)NR—, a provided compound is of Formula (XX^{D}-b) or Formula (XX^{D}-b′):

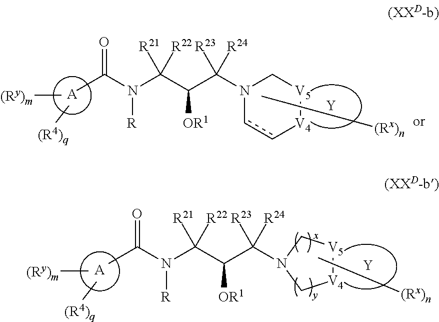

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (I^{D}) or Formula (I^{D′}), wherein L_{D} is linker L_{B}, and L_{B} is —C(O)NR—, and R²¹-R²⁴ is hydrogen, a provided compound is of Formula (IV^{D}-c) or Formula (IV^{D}-c′):

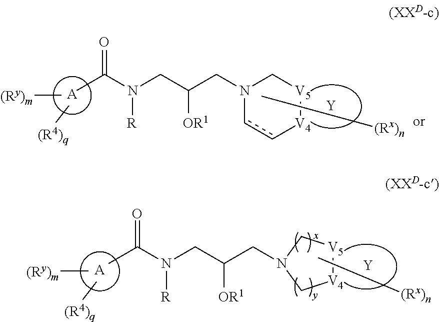

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (I^{D}) or Formula (I^{D′}), wherein L_{D} is —O— and R²¹-R²⁴ is hydrogen, a provided compound is of Formula (XX^{D}-d) or Formula (XX^{D}-d′):

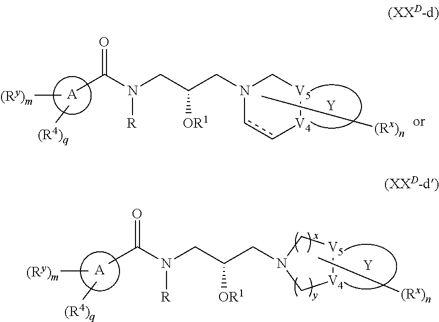

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (I^{D}) or Formula (I^{D′}), wherein L_{D} is —O— and R²¹-R²⁴ is hydrogen, a provided compound is of Formula (XX^{D}-e) or Formula (XX^{D}-e′):

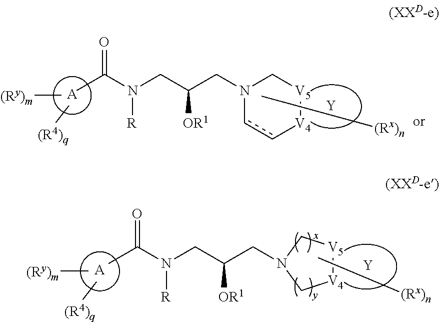

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (A-I^{D}), wherein Ring A is a monocyclic aromatic ring having 0, 1, 2, or 3 nitrogen heteroatoms:

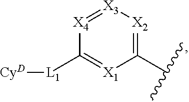

a provided compound is of Formula (A-V^{D}):

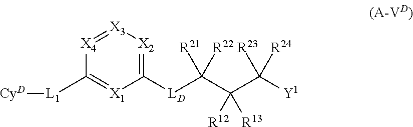

or a pharmaceutically acceptable salt thereof, wherein X₁, X₂, X₃, and X₄ are independently selected from the group consisting of N, CH, and CR^{y}, provided that at least one of X₂, X₃, and X₄ is not N.

In certain embodiments of Formula (A-V^{D}), wherein R¹² is hydrogen and R¹³ is —OR¹, a provided compound is of Formula (V^{D}) or Formula (V^{D′}):

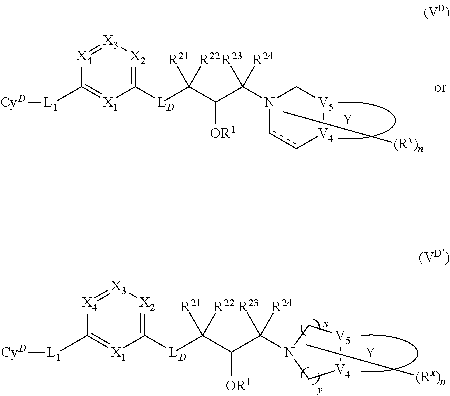

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{D}) or Formula (V^{D′}), a provided compound is of Formula (V^{D}-a) Formula (V^{D}-a′):

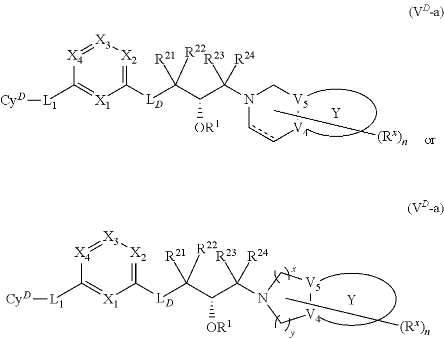

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{D}) or Formula (V^{D′}), a provided compound is of Formula (V^{D}-b) or Formula (V^{D}-b′):

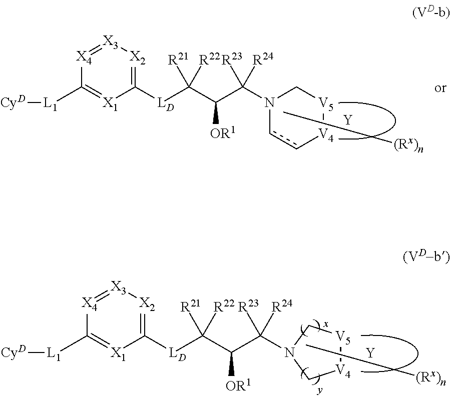

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{D}) or Formula (V^{D′}), wherein R²¹-R²⁴ is hydrogen, a provided compound is of Formula (V^{D}-c) or Formula (V^{D}-c′):

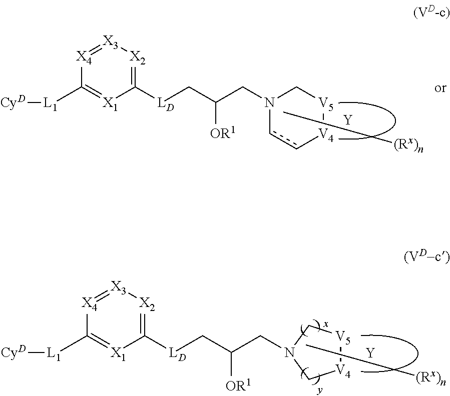

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{D}) or Formula (V^{D′}), wherein R²¹R²⁴ is hydrogen, a provided compound is of Formula (V^{D}-d) or Formula (V^{D}-d′):

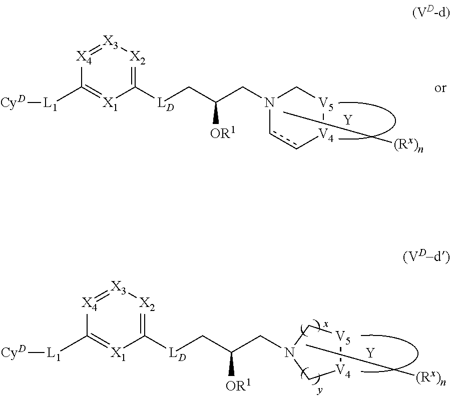

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{D}) or Formula (V^{D′}), wherein R²¹-R²⁴ is hydrogen, a provided compound is of Formula (V^{D}-e) or Formula (V^{D}-e′):

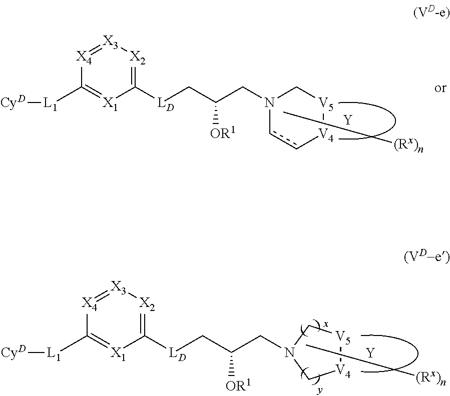

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{D}) or Formula (V^{D′}), wherein each of X₁, X₂, X₃, and X₄ is CH, a provided compound is of Formula (VI^{D}) or Formula (VI^{D′}):

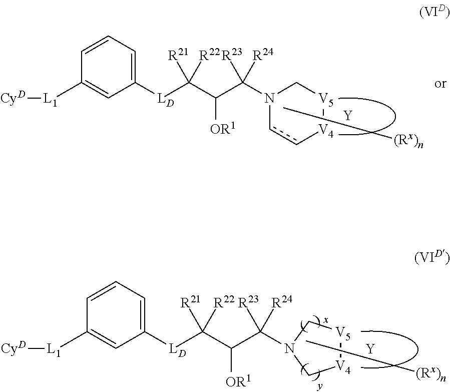

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{D}) or Formula (V^{D′}), wherein each of X₁, X₂, X₃, and X₄ is CH, a provided compound is of Formula (VI^{D}-a) or Formula (VI^{D}-a′):

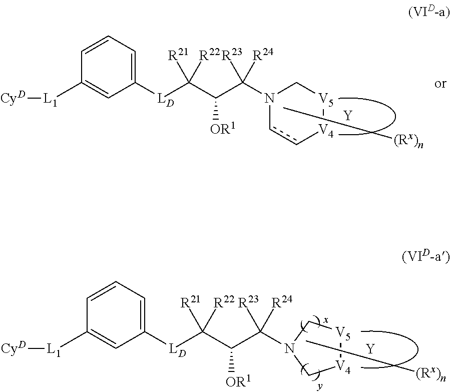

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{D}) or Formula (V^{D′}), wherein each of X₁, X₂, X₃, and X₄ is CH, a provided compound is of Formula (VI^{D}-b) or Formula (VI^{D}-b′):

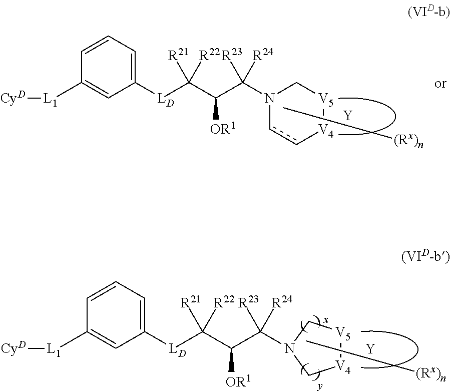

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{D}) or Formula (V^{D′}), wherein each of X₁, X₂, X₃, and X₄ is CH, and R²¹-R²⁴ is hydrogen, a provided compound is of Formula (VI^{D}-c) or Formula (VI^{D}-c′):

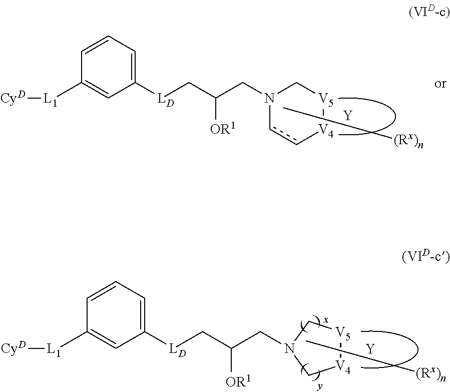

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{D}) or Formula (V^{D′}), wherein each of X₁, X₂, X₃, and X₄ is CH, and R²¹-R²⁴ is hydrogen, a provided compound is of Formula (VI^{D}-d) or Formula (VI^{D}-d′):

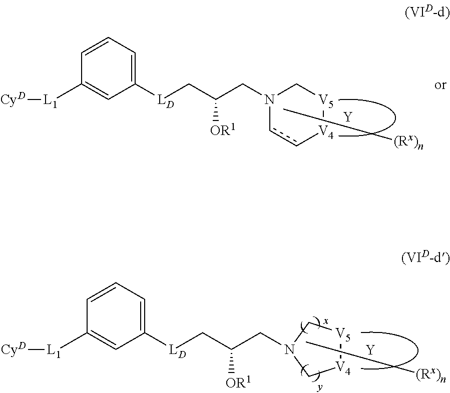

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{D}) or Formula (V^{D′}), wherein each of X₁, X₂, X₃, and X₄ is CH, and R²¹-R²⁴ is hydrogen, a provided compound is of Formula (VI^{D}-e) or Formula (VI^{D}-e′):

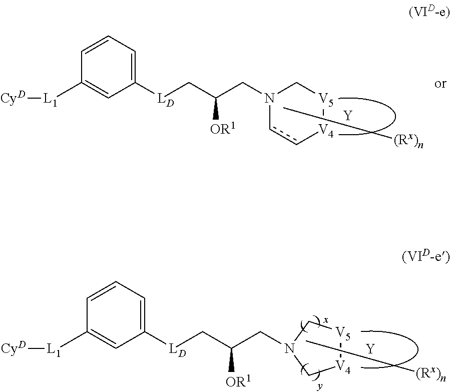

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{D}) or Formula (V^{D′}), wherein X₁ is N, and each of X₂, X₃, and X₄ is CH, a provided compound is of Formula (VII^{D}) or Formula (VII^{D′}):

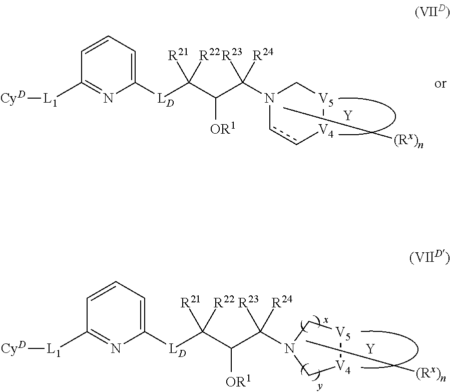

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{D}) or Formula (V^{D′}), wherein X₁ is N, and each of X₂, X₃, and X₄ is CH, a provided compound is of Formula (VII^{D}-a) or Formula (VII^{D}-a′):

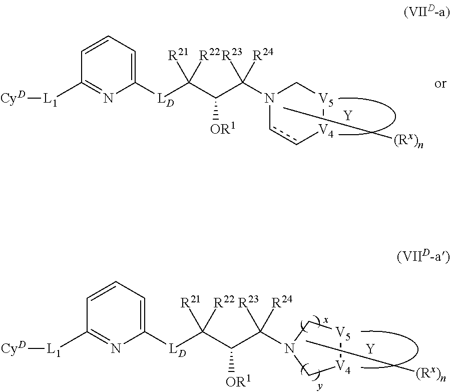

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{D}) or Formula (V^{D′}), wherein X₁ is N, and each of X₂, X₃, and X₄ is CH, a provided compound is of Formula (VII^{D}-b) or Formula (VII^{D}-b′):

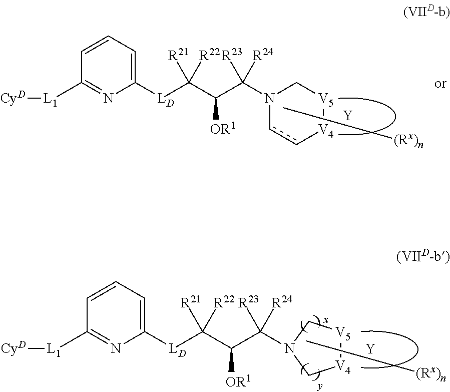

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{D}) or Formula (V^{D′}), wherein X₁ is N, each of X₂, X₃, and X₄ is CH, and R²¹-R²⁴ is hydrogen, a provided compound is of Formula (VII^{D}-c) or Formula (VII^{D}-c′):

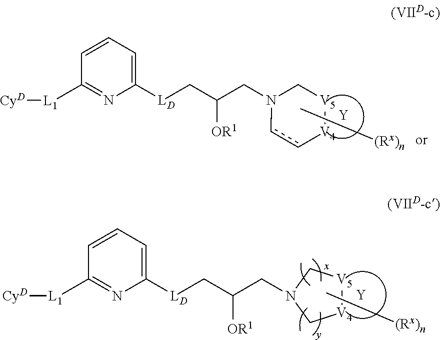

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{D}) or Formula (V^{D′}), wherein X₁ is N, each of X₂, X₃, and X₄ is CH, and R²¹-R²⁴ is hydrogen, a provided compound is of Formula (VII^{D-}d) or Formula (VII^{D}-d′):

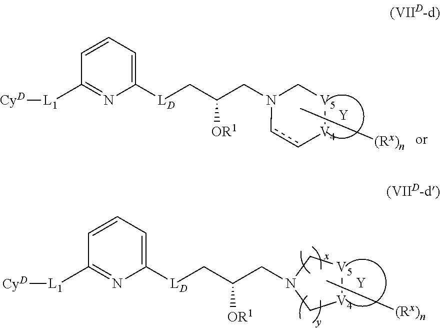

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{D}) or Formula (V^{D′}), wherein X₁ is N, each of X₂, X₃, and X₄ is CH, and R²¹-R²⁴ is hydrogen, a provided compound is of Formula (VII^{D-}e) or Formula (VII^{D}-e′):

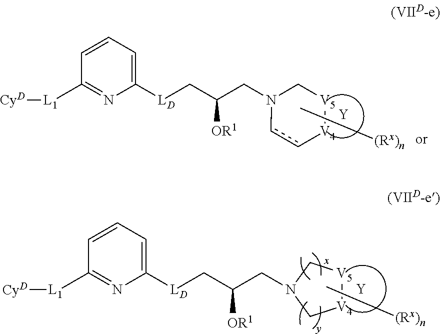

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{D}) or Formula (V^{D′}), wherein X₂ is N, and each of X₁, X₃, and X₄ is CH, a provided compound is of Formula (VIII^{D}) or Formula (VIII^{D′}):

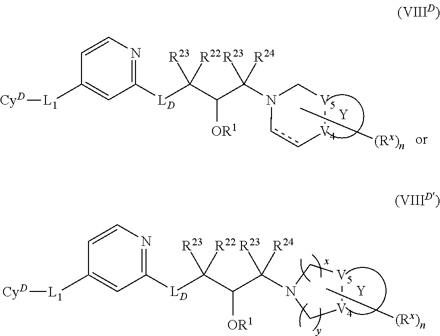

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{D}) or Formula (V^{D′}), wherein X₂ is N, and each of X₁, X₃, and X₄ is CH, a provided compound is of Formula (VIII^{D}-a) or Formula (VIII^{D}-a′):

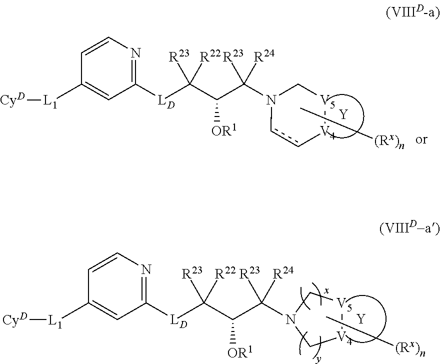

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{D}) or Formula (V^{D′}), wherein X₂ is N, and each of X₁, X₃, and X₄ is CH, a provided compound is of Formula (VIII^{D}-b) or Formula (VIII^{D}-b′):

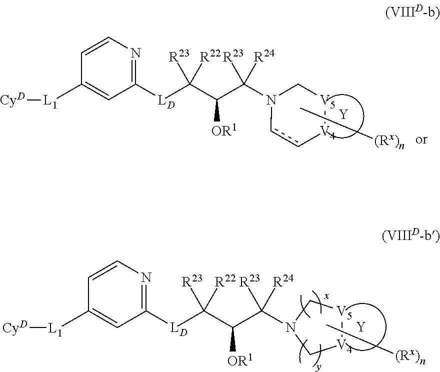

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{D}) or Formula (V^{D′}), wherein X₂ is N, each of X₁, X₃, and X₄ is CH, and R²¹-R²⁴ is hydrogen, a provided compound is of Formula (VIII^{D}-c) or Formula (VIII^{D}-c′):

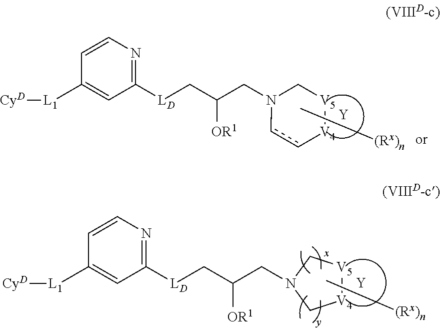

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{D}) or Formula (V^{D′}), wherein X₂ is N, each of X₁, X₃, and X₄ is CH, and R²¹-R²⁴ is hydrogen, a provided compound is of Formula (VIII^{D}-d) or Formula (VIII^{D}-d′):

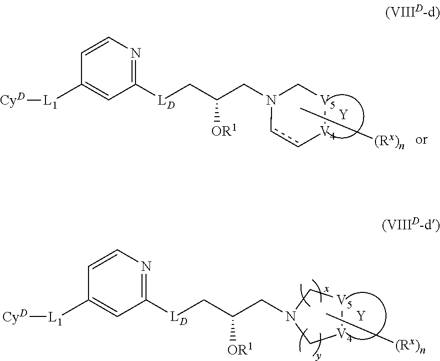

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{D}) or Formula (V^{D′}), wherein X₂ is N, each of X₁, X₃, and X₄ is CH, and R²¹-R²⁴ is hydrogen, a provided compound is of Formula (VIII^{D}-e) or Formula (VIII^{D}-e′):

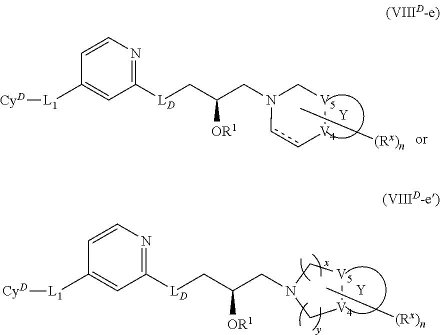

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{D}) or Formula (V^{D′}), wherein X₄ is N, and each of X₁, X₂, and X₃ is CH, a provided compound is of Formula (XIII^{D}) or Formula (XIII^{D′}):

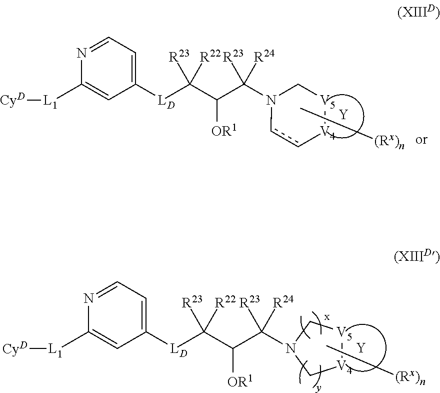

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{D}) or Formula (V^{D′}), wherein X₂ is N, and each of X₁, X₃, and X₄ is CH, a provided compound is of Formula (VIII^{D}-a) or Formula (VIII^{D}-a′):

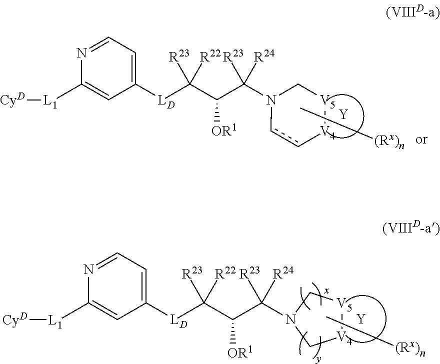

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{D}) or Formula (V^{D′}), wherein X₂ is N, and each of X₁, X₃, and X₄ is CH, a provided compound is of Formula (VIII^{D}-b) or Formula (VIII^{D}-b′):

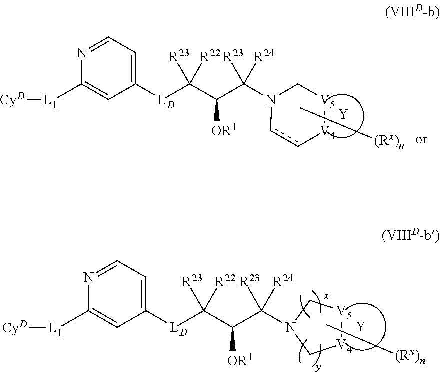

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{D}) or Formula (V^{D′}), wherein X₂ is N, each of X₁, X₃, and X₄ is CH, and R²¹-R²⁴ is hydrogen, a provided compound is of Formula (VIII^{D}-c) or Formula (VIII^{D}-c′):

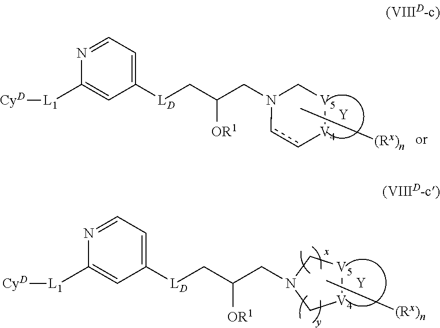

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{D}) or Formula (V^{D′}), wherein X₂ is N, each of X₁, X₃, and X₄ is CH, and R²¹-R²⁴ is hydrogen, a provided compound is of Formula (VIII^{D}-d) or Formula (VIII^{D}-d′):

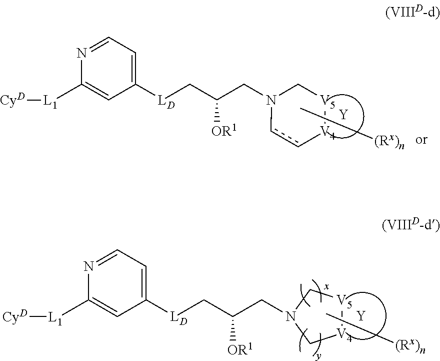

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{D}) or Formula (V^{D′}), wherein X₂ is N, each of X₁, X₃, and X₄ is CH, and R²¹-R²⁴ is hydrogen, a provided compound is of Formula (VIII^{D}-e) or Formula (VIII^{D}-e′):

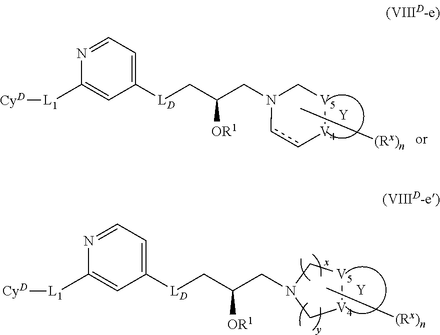

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{D}) or Formula (V^{D′}), wherein each of X₁ and X₂ is N, and each of X₃ and X₄ is CH, a provided compound is of Formula (IX^{D}) or Formula (IX^{D′}):

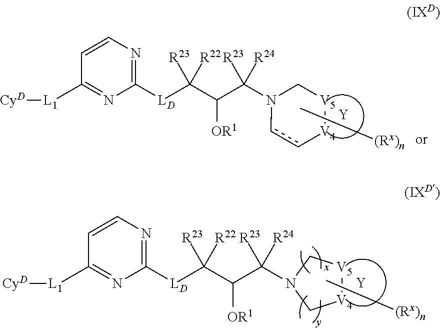

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{D}) or Formula (V^{D′}), wherein each of X₁ and X₂ is N, and each of X₃ and X₄ is CH, a provided compound is of Formula (IX^{D}-a) or Formula IX^{D}-a′):

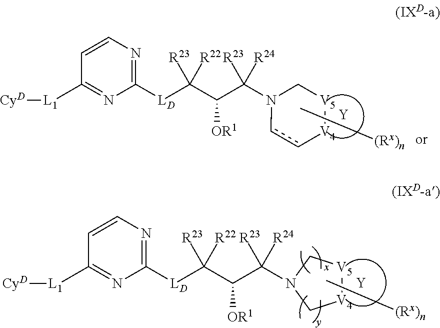

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{D}) or Formula (V^{D′}), wherein each of X₁ and X₂ is N, and each of X₃ and X₄ is CH, a provided compound is of Formula (IX^{D}-b) or Formula (IX^{D}-b′):

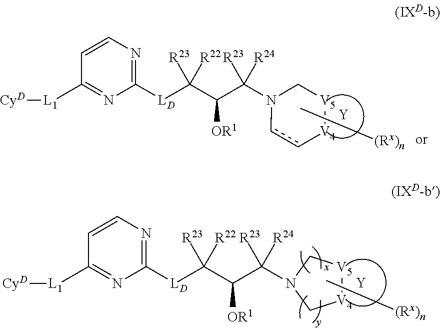

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{D}) or Formula (V^{D′}), wherein each of X₁ and X₂ is N, each of X₃ and X₄ is CH, and R²¹-R²⁴ is hydrogen, a provided compound is of Formula (IX^{D}-c) or Formula (IX^{D}-c′):

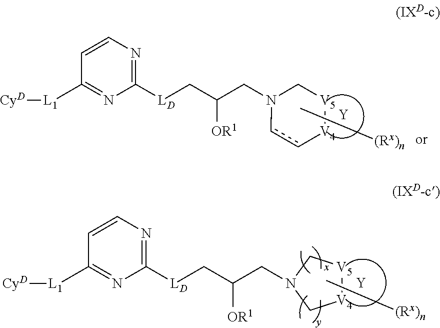

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{D}) or Formula (V^{D′}), wherein each of X₁ and X₂ is N, each of X₃ and X₄ is CH, and R²¹-R²⁴ is hydrogen, a provided compound is of Formula (IX^{D}-d) or Formula (IX^{D}-d′):

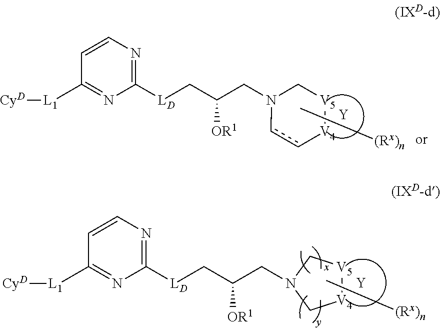

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{D}) or Formula (V^{D′}), wherein each of X₁ and X₂ is N, each of X₃ and X₄ is CH, and R²¹-R²⁴ is hydrogen, a provided compound is of Formula (IX^{D}-e) or Formula (IX^{D}-e′):

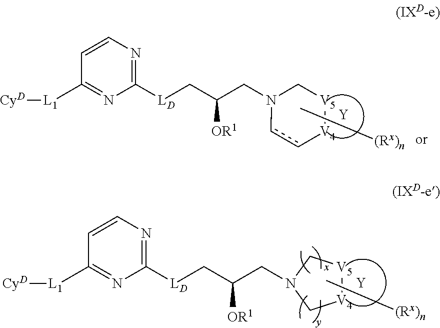

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{D}) or Formula (V^{D′}), wherein each of X₁ and X₃ is N, and each of X₂ and X₄ is CH, a provided compound is of Formula (X^{D}) or Formula (X^{D′}):

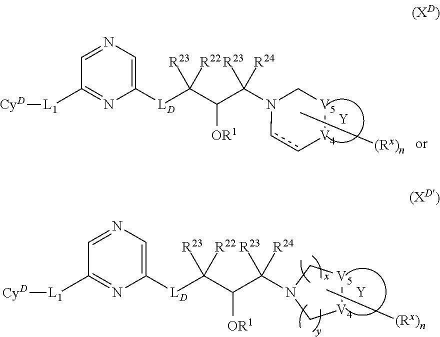

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{D}) or Formula (V^{D′}), wherein each of X₁ and X₃ is N, and each of X₂ and X₄ is CH, a provided compound is of Formula (X^{D}-a) or Formula (X^{D}-a′):

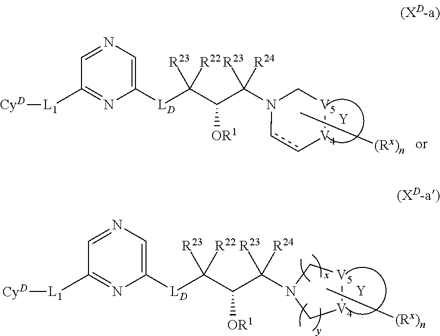

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{D}) or Formula (V^{D′}), wherein each of X₁ and X₃ is N, and each of X₂ and X₄ is CH, a provided compound is of Formula (X^{D}-b) or Formula (X^{D}-b′):

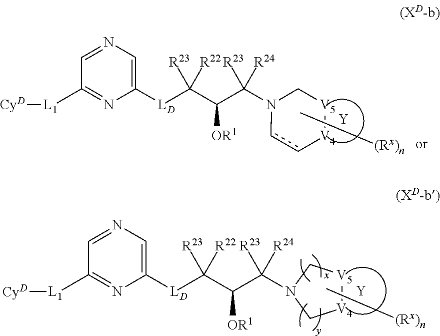

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{D}) or Formula (V^{D′}), wherein each of X₁ and X₃ is N, each of X₂ and X₄ is CH, and R²¹-R²⁴ is hydrogen, a provided compound is of Formula (X^{D}-c) or Formula (X^{D}-c′):

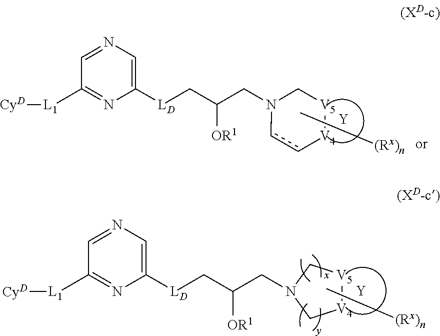

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{D}) or Formula (V^{D′}), wherein each of X₁ and X₃ is N, each of X₂ and X₄ is CH, and R²¹-R²⁴ is hydrogen, a provided compound is of Formula (X^{D}-d) or Formula (X^{D}-d′):

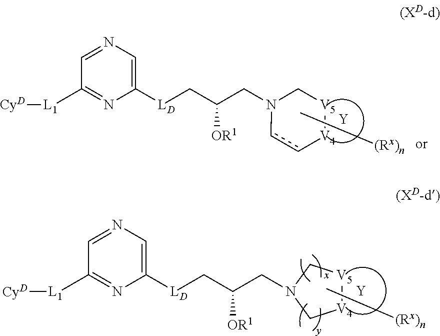

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{D}) or Formula (V^{D′}), wherein each of X₁ and X₃ is N, each of X₂ and X₄ is CH, and R²¹-R²⁴ is hydrogen, a provided compound is of Formula (X^{D}-e) or Formula (X^{D}-e′):

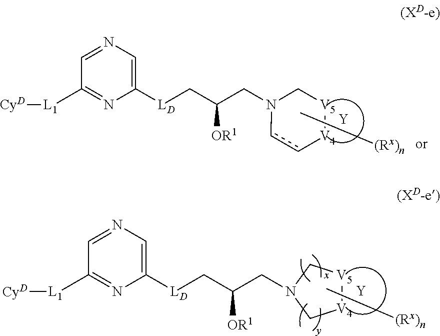

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{D}) or Formula (V^{D′}), wherein each of X₂ and X₄ is N, and each of X₁ and X₃ is CH, a provided compound is of Formula (XI^{D}) or Formula (XI^{D′}):

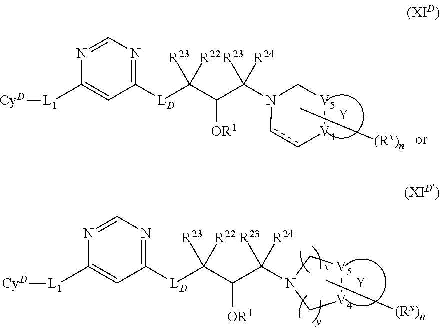

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{D}) or Formula (V^{D′}), wherein each of X₂ and X₄ is N, and each of X₁ and X₃ is CH, a provided compound is of Formula (XI^{D}-a) or Formula (XI^{D}-a′):

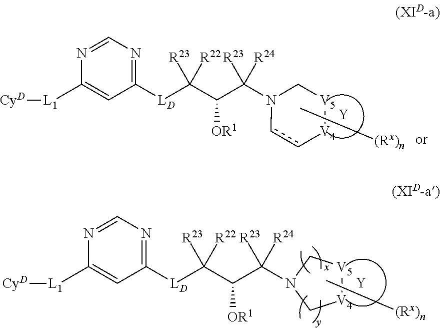

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{D}) or Formula (V^{D′}), wherein each of X₂ and X₄ is N, and each of X₁ and X₃ is CH, a provided compound is of Formula (XI^{D}-b) or Formula (XI^{D}-b′):

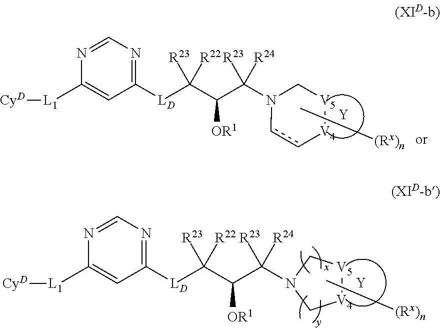

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{D}) or Formula (V^{D′}), wherein each of X₂ and X₄ is N, and each of X₁ and X₃ is CH, and R²¹-R²⁴ is hydrogen, a provided compound is of Formula (XI^{D}-c) or Formula (XI^{D}-c′):

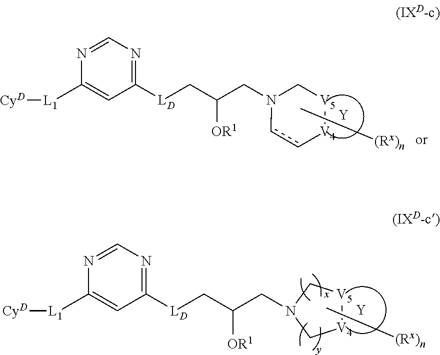

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{D}) or Formula (V^{D′}), wherein each of X₂ and X₄ is N, and each of X₁ and X₃ is CH, and R²¹-R²⁴ is hydrogen, a provided compound is of Formula (XI^{D}-d) or Formula (XI^{D}-d′):

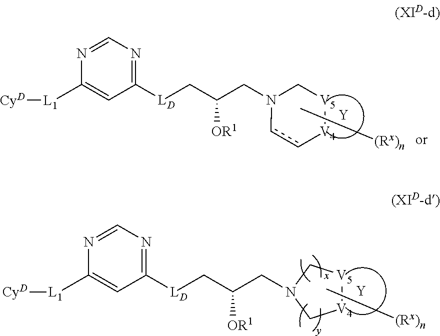

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{D}) or Formula (V^{D′}), wherein each of X₂ and X₄ is N, and each of X₁ and X₃ is CH, and R²¹-R²⁴ is hydrogen, a provided compound is of Formula (XI^{D}-e) or Formula (XI^{D}-e′):

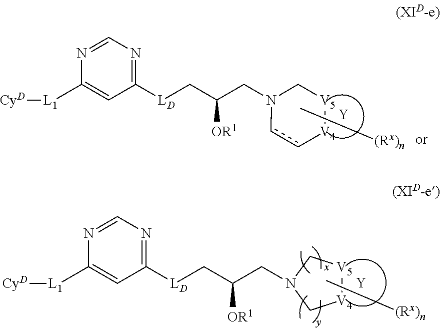

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{D}) or Formula (V^{D′}), wherein each of X₁ and X₄ is N, and each of X₂ and X₃ is CH, a provided compound is of Formula (XII^{D}) or Formula (XII^{D′}):

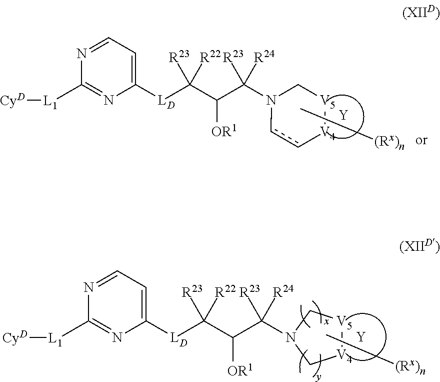

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{D}) or Formula (V^{D′}), wherein each of X₁ and X₄ is N, and each of X₂ and X₃ is CH, a provided compound is of Formula (XII^{D}-a) or Formula (XII^{D}-a′):

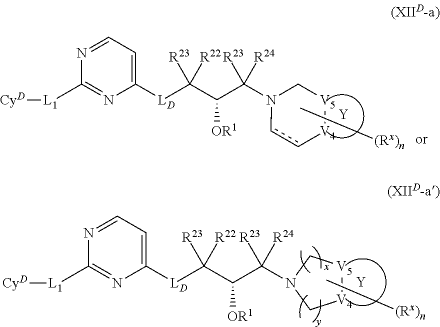

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{D}) or Formula (V^{D′}), wherein each of X₁ and X₄ is N, and each of X₂ and X₃ is CH, a provided compound is of Formula (XII^{D}-b) or Formula (XII^{D}-b′):

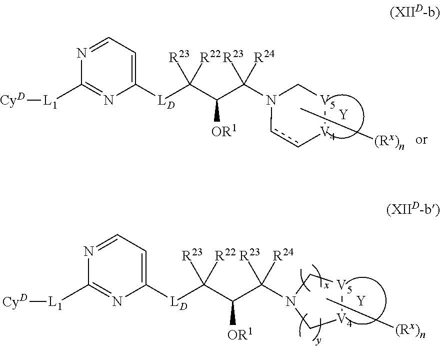

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{D}) or Formula (V^{D′}), wherein each of X₁ and X₄ is N, and each of X₂ and X₃ is CH, and R²¹-R²⁴ is hydrogen, a provided compound is of Formula (XII^{D}-c) or Formula (XII^{D}-c′):

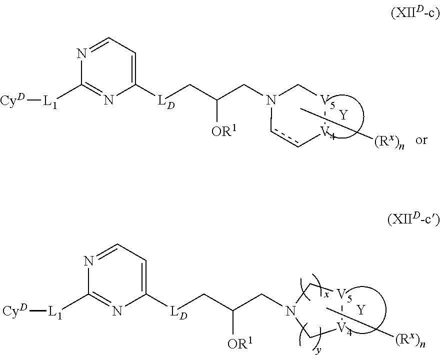

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{D}) or Formula (V^{D′}), wherein each of X₁ and X₄ is N, and each of X₂ and X₃ is CH, and R²¹-R²⁴ is hydrogen, a provided compound is of Formula (XII^{D}-d) or Formula (XII^{D}-d′):

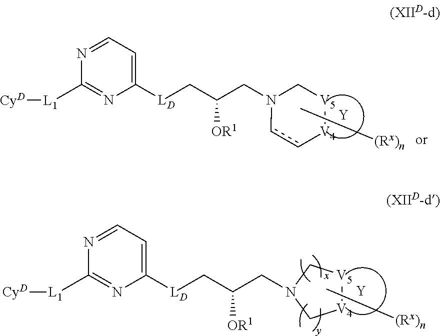

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

In certain embodiments of Formula (V^{D}) or Formula (V^{D′}), wherein each of X₁ and X₄ is N, and each of X₂ and X₃ is CH, and R²¹-R²⁴ is hydrogen, a provided compound is of Formula (IX^{D}-e) or Formula (IX^{D}-e′):

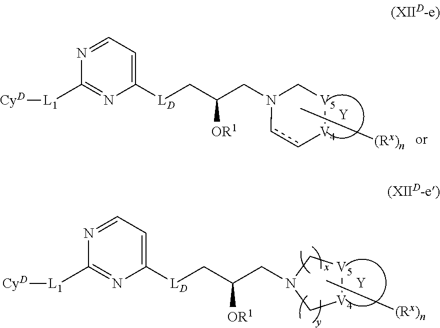

or a pharmaceutically acceptable salt thereof.

In certain embodiments, V₄ is C and V₅ is C. In certain embodiments, V₄ is C and V₅ is N. In certain embodiments, V₄ is N and V₅ is C. In certain embodiments, V₄ is N and V₅ is N.

As defined generally herein, Y¹ is of formula (x) or formula (y)

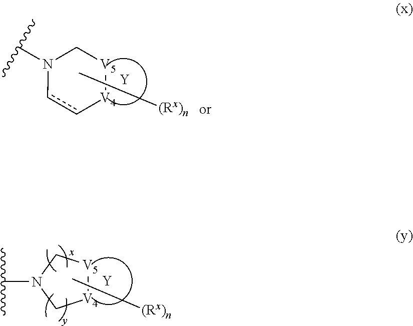

wherein V₄, V₅, Ring Y, R^{x} and n are as defined herein.

As defined generally herein, Ring Y is a 5- to 6-membered heteroaryl ring.

In some embodiments, Y¹ is of formula (x):

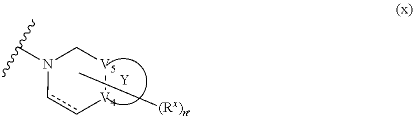

as provided in the above compounds of Formula (A), (I), and various subgenera thereof, and represents a 5,6-bicyclic ring system (wherein Ring Y is a 5-membered heteroaryl ring) or a 6,6-bicyclic ring system (wherein Ring Y is a 6-membered heteroaryl ring), and wherein V₄ and V₅ at the point of fusion between the two rings is each independently N or C.

In some embodiments, Y¹ is of formula (y):

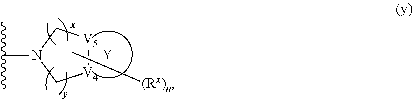

as provided in the above compounds of Formula (A), (I), and various subgenera thereof, and represents a 5,5-a 5,6-, or a 5,7-bicyclic ring system (wherein Ring Y is a 5-membered heteroaryl ring) or a 6,5-, 6,6-, or 6,7-bicyclic ring system (wherein Ring Y is a 6-membered heteroaryl ring), and wherein V₄ and V₅ at the point of fusion between the two rings is each independently N or C.

One of ordinary skill in the art will appreciate that an R^{x} group can be attached anywhere on the bicyclic ring system Y¹. In certain embodiments, one or more R^{x} groups are attached to the first ring of Y¹ (the first ring corresponds to the ring comprising the nitrogen point of attachment to the parent molecule, e.g., a 1,2-dihydropyridinyl ring, 1,2,3,6-tetrahydropyridinyl ring, 1,2,3,5-tetrahydropyrrolyl ring, 1,2,5,6-tetrahydropyrimidinyl ring, 1,2,3,6-tetrahydropyrazinyl ring, 3,4,5,6-tetrahydro-1,2,4-triazinyl ring, and the like). In certain embodiments, one or more R^{x} groups are attached to Ring Y. In certain embodiments, R^{x} groups are attached to both rings of the bicyclic ring system Y¹. In certain embodiments, the bicyclic ring system Y¹ is optionally substituted with (R^{x})ₙ, with the proviso that when the first ring of the bicyclic ring system Y¹ is substituted at one of the positions alpha to the nitrogen, R^{x} is not optionally substituted aryl, optionally substituted acyl, optionally substituted carboxylate, or optionally substituted amide. In certain embodiments, the first ring of the bicyclic ring system Y¹ does not comprise an R^{x} substituent. In certain embodiments, only Ring Y is optionally substituted with (R^{x})ₙ.

In certain embodiments, Y¹ is a bicyclic ring system of formula (x-1)

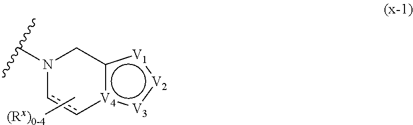

wherein:

each instance of V₁, V₂, and V₃ is independently O, S, N, NH, NR^{x}, CH, or CR^{x}; and

V₄ is N or C, wherein R^{x} is as defined herein.

In certain embodiments of formula (x-1), V₄ is N and Y¹ is of formula (x-1a):

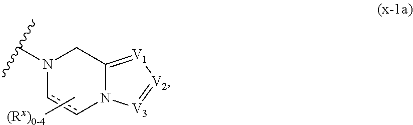

wherein R^{x}, V₁, V₂, and V₃ are as defined herein.

In certain embodiments of formula (x-1a), Y¹ is of formula (x-1b):

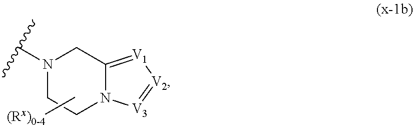

wherein R^{x}, V₁, V₂, and V₃ are as defined herein.

As generally defined herein, V₁ is independently O, S, N, NH, NR^{x}, CH, or CR^{x}, as valency permits. In certain embodiments of formula (x-1a) and formula (x-1b), V₁ is N. In certain embodiments of formula (x-1a) and formula (x-1b), V₁ is CH. In certain embodiments of formula (x-1a) and formula (x-1b), V₁ is CR^{x}.

As generally defined herein, V₂ is independently O, S, N, NH, NR^{x}, CH, or CR^{x}, as valency permits. In certain embodiments of formula (x-1a) and formula (x-1b), V₂ is N. In certain embodiments of formula (x-1a) and formula (x-1b), V₂ is CH. In certain embodiments of formula (x-1a) and formula (x-1b), V₂ is CR^{x}.

As generally defined herein, V₃ is independently O, S, N, NH, NR^{x}, CH, or CR^{x}, as valency permits. In certain embodiments of formula (x-1a) and formula (x-1b), V₃ is N. In certain of formula (x-1a) and formula (x-1b), V₃ is CH. In certain embodiments of formula (x-1a) and formula (x-1b), V₃ is CR^{x}.

In certain embodiments of formula (x-1a) and formula (x-1b), V₁ is N and V₂ is CR^{x}. In certain embodiments of formula (x-1a) and formula (x-1b), V₁ is N and V₂ is CH. In certain embodiments of formula (x-1a) and formula (x-1b), V₁ is N and V₂ is N. In certain embodiments of formula (x-1a) and formula (x-1b), V₁ is N and V₃ is CR^{x}. In certain embodiments of formula (x-1a) and formula (x-1b), V₁ is N and V₃ is CH. In certain embodiments of formula (x-1a) and formula (x-1b), V₁ is N and V₃ is N. In certain embodiments of formula (x-1a) and formula (x-1b), V₂ is N and V₃ is CR^{x}. In certain embodiments of formula (x-1a) and formula (x-1b), V₂ is N and V₃ is CH. In certain embodiments of formula (x-1a) and formula (x-1b), V₂ is N and V₃ is N.

In certain embodiments of formula (x-1a) and formula (x-1b), V₁ is N; V₂ is CR^{x}; and V₃ is N, CH, or CR^{x}. In certain embodiments of formula (x-1a) and formula (x-1b), V₁ is N; V₂ is CH; and V₃ is N, CH, or CR^{x}. In certain embodiments of formula (x-1a) and formula (x-1b), V₁ is N; V₂ is N; and V₃ is N, CH, or CR^{x}. In certain embodiments of formula (x-1a) and formula (x-1b), V₁ is N; V₃ is CR^{x}; and V₂ is N, CH, or CR^{x}. In certain embodiments of formula (x-1a) and formula (x-1b), V₁ is N; V₃ is CH; and V₂ is N, CH, or CR^{x}. In certain embodiments of formula (x-1a) and formula (x-1b), V₁ is N; V₃ is N; and V₂ is N, CH, or CR^{x}. In certain embodiments of formula (x-1a) and formula (x-1b), V₂ is N; V₃ is CR^{x}; and V₁ is N, CH, or CR^{x}. In certain embodiments of formula (x-1a) and formula (x-1b), V₂ is N; V₃ is CH; and V₁ is N, CH, or CR^{x}. In certain embodiments of formula (x-1a) and formula (x-1b), V₂ is N; V₃ is N; and V₁ is N, CH, or CR^{x}.

Exemplary compounds of formula (x-1a) and formula (x-1b) include, but are not limited to:

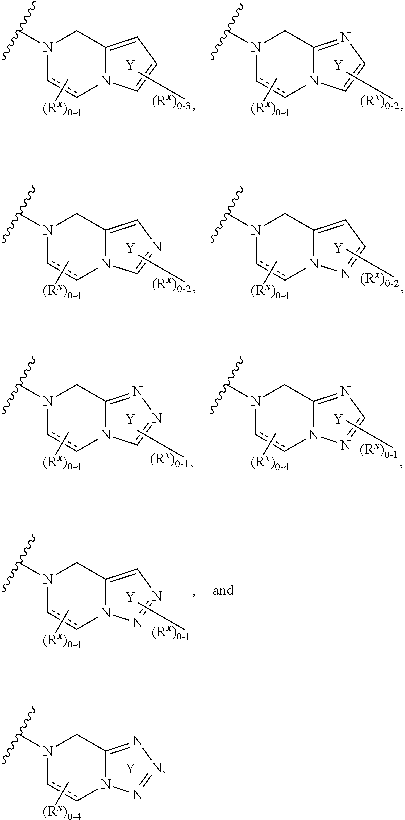

wherein the 4,5,6,7-tetrahydro-pyrazinyl ring or 4,5-dihydro-pyrazineyl ring of the 5,6-fused ring system comprises 0, 1, 2, 3, or 4 R^{x} substituents, and Ring Y of the 5,5-fused ring system comprises 0, 1, 2, or 3 R^{x} substituents.

Exemplary compounds of formula (x-1a) and formula (x-1b) include, but are not limited to:

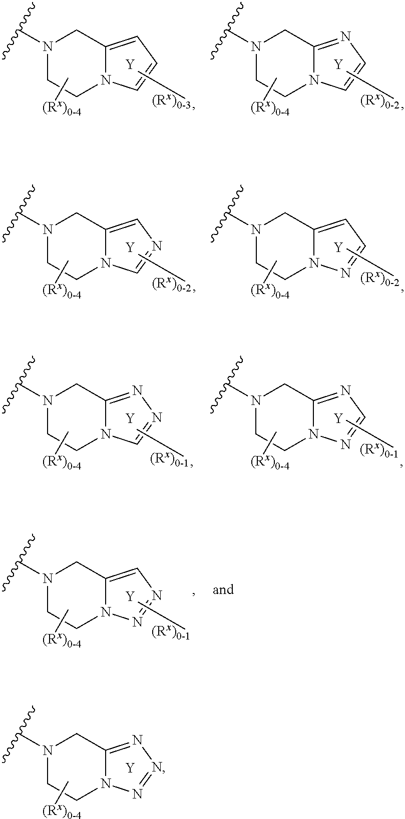

wherein the 4,5,6,7-tetrahydro-pyrazinyl ring of the bicyclic ring system comprises 0, 1, 2, 3, or 4 R^{x} substituents, and Ring Y of the bicyclic ring system comprises 0, 1, 2, or 3 R^{x} substituents.

In certain embodiments of formula (x-1), V₄ is C and Y¹ is of formula (i), (ii), or (iii):

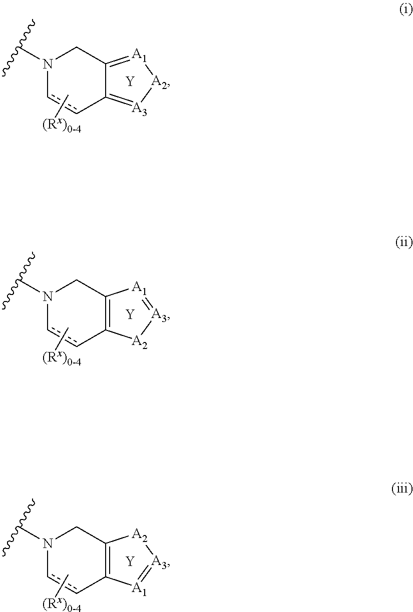

wherein each instance of A₁ and A₃ is independently N, CH, or CR^{x}, and A₂ is O, S, NH, or NR^{x}, wherein R^{x} is as defined herein.

In certain embodiments, [symbol] represents a double bond. However, in certain embodiments, [symbol] represents a single bond, as provided in formula (i-a), (ii-a), and (iii-a):

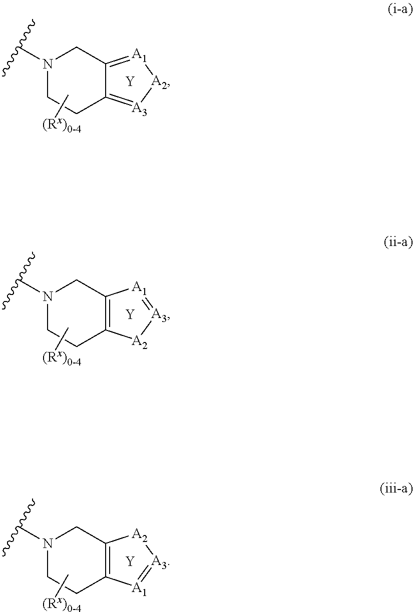

In certain embodiments of formula (i), (ii), or (iii), A₁ is N. In certain embodiments, Aₜ is CH. In certain embodiments, Aₜ is CR^{x}. In certain embodiments, A₃ is N. In certain embodiments, A₃ is CH. In certain embodiments, A₃ is CR^{x}. In certain embodiments, A₂ is O. In certain embodiments, A₂ is S. In certain embodiments, A₂ is NH. In certain embodiments, A₂ is NR^{x}.

In certain embodiments, A₁ is N and A₂ is O. In certain embodiments, A₁ is CH or CR^{x} and A₂ is O. In certain embodiments, A₁ is N and A₂ is S. In certain embodiments, A₁ is CH or CR^{x} and A₂ is S. In certain embodiments, A₁ is N and A₂ is NH or NR^{x}. In certain embodiments, A₁ is CH or CR^{x} and A₂ is NH or NR^{x}. In certain embodiments, A₁ is N and A₃ is N. In certain embodiments, A₁ is CH or CR^{x} and A₃ is N. In certain embodiments, A₁ is N and A₃ is CH or CR^{x}. In certain embodiments, A₁ is CH or CR^{x} and A₃ is CH or CR^{x}. In certain embodiments, A₃ is N and A₂ is O. In certain embodiments, A₃ is CH or CR^{x} and A₂ is O. In certain embodiments, A₃ is N and A₂ is S. In certain embodiments, A₃ is CH or CR^{x} and A₂ is S. In certain embodiments, A₃ is N and A₂ is NH or NR^{x}. In certain embodiments, A₃ is CH or CR^{x} and A₂ is NH or NR^{x}.

In certain embodiments, Ring Y is unsubstituted (i.e., does not comprise an R^{x} substituent). However, in certain embodiments, Ring Y is substituted with at least one R^{x} group. In certain embodiments, Ring Y is substituted with two R^{x} groups. In certain embodiments, Ring Y is substituted with three R^{x} groups.

Exemplary ring systems of formula (i) include, but are not limited to:

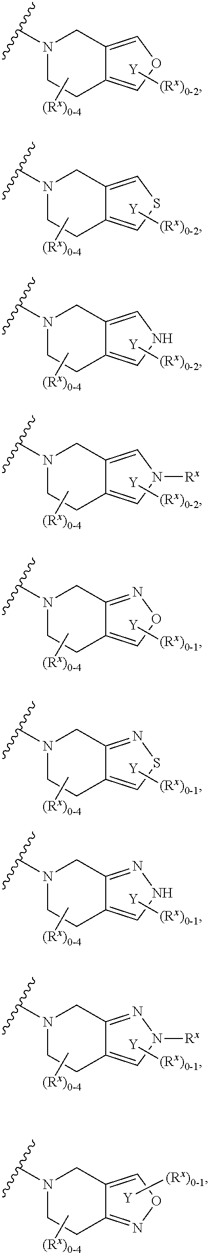

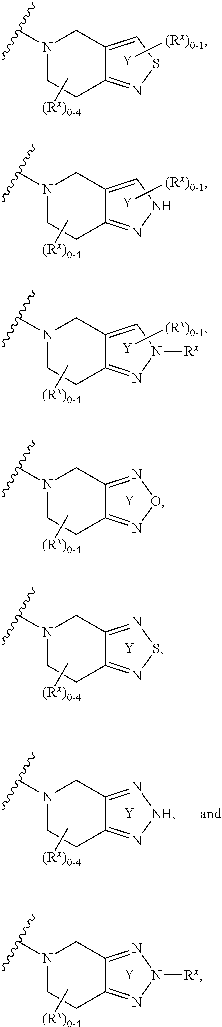

wherein the 1,2,3,6-tetrahydropyridinyl ring of the bicyclic ring system comprises 0, 1, 2, 3, or 4 R^{x} substituents, and Ring Y of the bicyclic ring system comprises 0, 1, 2, or 3 R^{x} substituents.

Exemplary ring systems of formula (ii) include, but are not limited to:

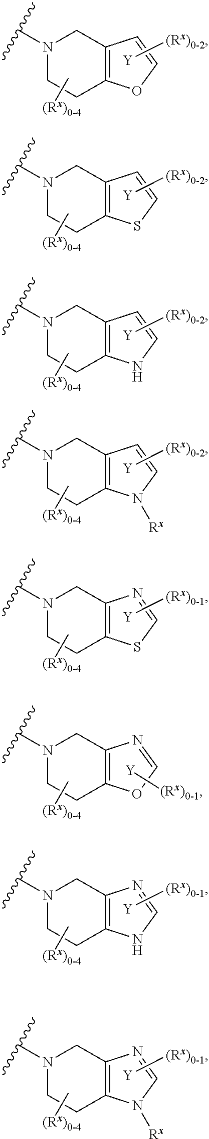

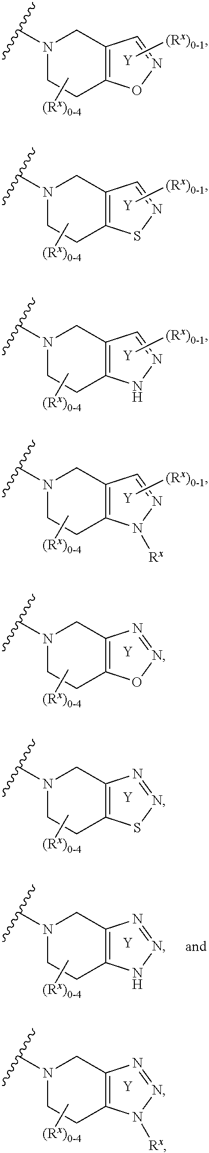

wherein the 1,2,3,6-tetrahydropyridinyl ring of the bicyclic ring system comprises 0, 1, 2, 3, or 4 R^{x} substituents, and Ring Y of the bicyclic ring system comprises 0, 1, 2, or 3 R^{x} substituents.

Exemplary ring systems of formula (iii) include, but are not limited to:

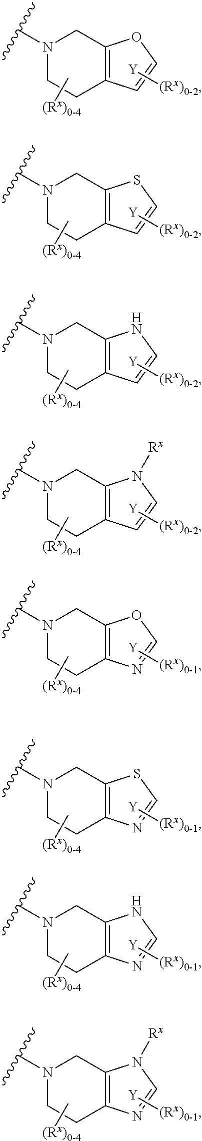

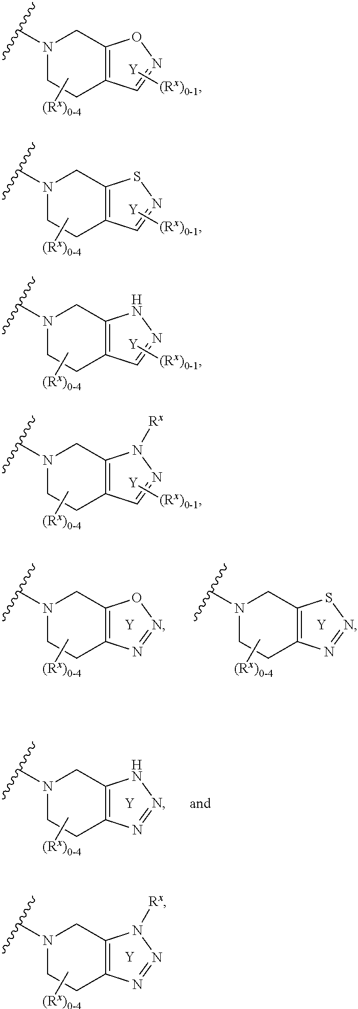

wherein the 1,2,3,6-tetrahydropyridinyl ring of the bicyclic ring system comprises 0, 1, 2, 3, or 4 R^{x} substituents, and Ring Y of the bicyclic ring system comprises 0, 1, 2, or 3 R^{x} substituents.

In certain embodiments, Y¹ is a bicyclic ring system of formula (iv):

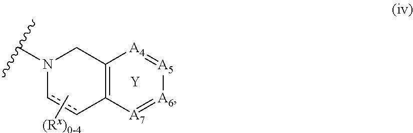

wherein each instance of A₄, A₅, A₆, and A₇ is independently N, CH, or CR^{x}, provided at least one of A₄, A₅, A₆, and A₇ is N, and wherein R^{x} is as defined herein.

In certain embodiments, [symbol] represents a double bond. However, in certain embodiments, [symbol] represents a single bond, as provided in formula (iv-a):

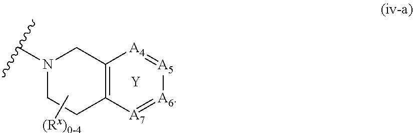

In certain embodiments, one of A₄, A₅, A₆, and A₇ is N. In certain embodiments, two of A₄, A₅, A₆, and A₇ is N.

In certain embodiments, A₄ is N. In certain embodiments, A₄ is CH. In certain embodiments, A₄ is CR^{x}. In certain embodiments, A₅ is N. In certain embodiments, A₅ is CH. In certain embodiments, A₅ is CR^{x}. In certain embodiments, A₆ is N. In certain embodiments, A₆ is CH. In certain embodiments, A₆ is CR^{x}. In certain embodiments, A₇ is N. In certain embodiments, A₇ is CH. In certain embodiments, A₇ is CR^{x}.

In certain embodiments, A₄ is N, and A₅, A₆, and A₇ are each independently CH or CR^{x}. In certain embodiments, A₅ is N, and A₄, A₆, and A₇ are each independently CH or CR^{x}. In certain embodiments, A₆ is N, and A₄, A₅, and A₇ are each independently CH or CR^{x}. In certain embodiments, A₇ is N, and A₄, A₅, and A₆ are each independently CH or CR^{x}. In certain embodiments, each of A₄ and A₅ is N, and A₆ and A₇ are each independently CH or CR^{x}. In certain embodiments, each of A₄ and A₆ is N, and A₅ and A₇ are each independently CH or CR^{x}. In certain embodiments, each of A₄ and A₇ is N, and A₅ and A₆ are each independently CH or CR^{x}. In certain embodiments, each of A₅ and A₆ is N, and A₄ and A₇ are each independently CH or CR^{x}. In certain embodiments, each of A₆ and A₇ is N, and A₄ and A₅ are each independently CH or CR^{x}. In certain embodiments, each of A₅ and A₇ is N, and A₄ and A₆ are each independently CH or CR^{x}.

In certain embodiments, Ring Y is unsubstituted (i.e., does not comprise an R^{x} substituent). However, in certain embodiments, Ring Y is substituted with at least one R^{x} group. In certain embodiments, Ring Y is substituted with two R^{x} groups. In certain embodiments, Ring Y is substituted with three R^{x} groups.

Exemplary ring systems of formula (iv) include, but are not limited to:

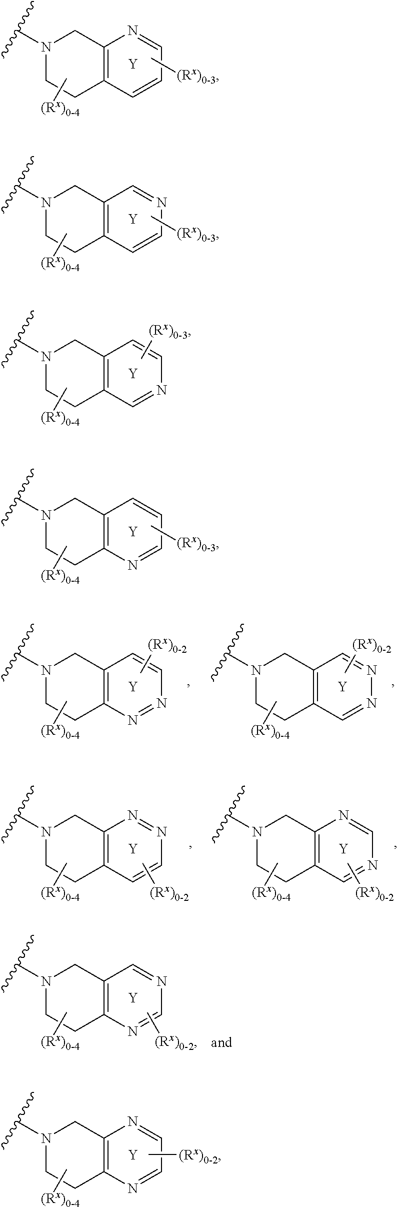

wherein Ring Y of the bicyclic ring system comprises 0, 1, 2, or 3 R^{x} substituents, and the 1,2,3,6-tetrahydropyridinyl ring of the bicyclic ring system comprises 0, 1, 2, 3, or 4 R^{x} substituents.

In certain embodiments, Y¹ is of formula (y):

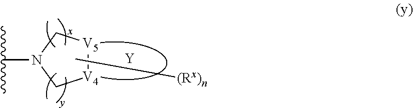

wherein V₄, V₅, Ring Y, n, x, y, and R^{x} are as defined herein.

In some embodiments of formula (y), wherein x is 0 and y is 2, 3, or 4, provided is a compound of Formula (y-a), (y-b), or (y-c):

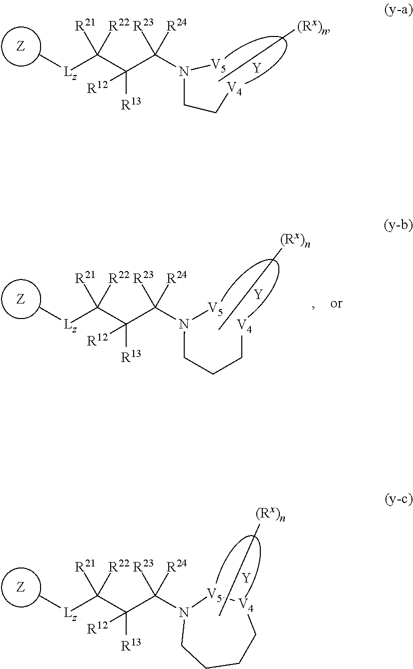

or a pharmaceutically acceptable salt thereof, wherein V₄, V₅, Ring Z, L_{z}, R², R³, R², R²², R²³, R²⁴, R^{x}, and n are as described herein.

In some embodiments of formula (y), wherein x is 1 and y is 1, provided is a compound of Formula (y-d):

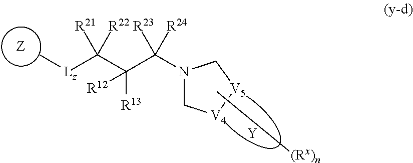

or a pharmaceutically acceptable salt thereof, wherein V₄, V₅, Ring Z, L_{z}, R¹², R¹³, R²¹, R²², R²³, R²⁴, R^{x}, and n are as described herein.

In some embodiments of formula (y), wherein x is 1 and y is 3, provided is a compound of Formula (y-e):

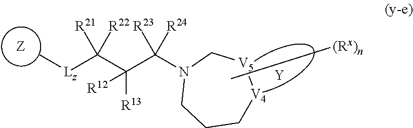

or a pharmaceutically acceptable salt thereof, wherein V₄, V₅, Ring Z, L_{z}, R¹², R¹³, R²¹, R²², R²³, R²⁴, R^{x}, and n are as described herein.

In certain embodiments of formula (A), Y¹ is a bicyclic ring system of formula (y-1)

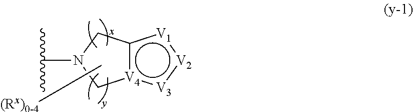

wherein

each instance of V₁, V₂, and V₃ is independently O, S, N, NH, NR^{x}, CH, or CR^{x};

V₄ is N or C; and

x is 0 and y is 2, 3, or 4; or

x is 1 and y is 1; or

x is 1 and y is 3.

In certain embodiments of formula (y-1), V₄ is N. In certain embodiments of formula (y-1), V₄ is C.

In certain embodiments of formula (y-1), V₄ is N and Y¹ is of formula (x-1a):

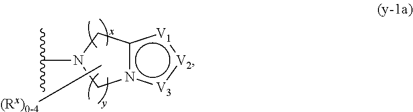

wherein x, y, R^{x}, V₁, V₂, and V₃ are as defined herein.

As generally defined herein, V₁ is independently O, S, N, NH, NR^{x}, CH, or CR^{x}, as valency permits. In certain embodiments of formula (y-1a), V₁ is N. In certain embodiments of formula (x-1a), V₁ is CH. In certain embodiments of formula (y-1a), V₁ is CR^{x}.

As generally defined herein, V₂ is independently O, S, N, NH, NR^{x}, CH, or CR^{x}, as valency permits. In certain embodiments of formula (y-1a), V₂ is N. In certain embodiments of formula (y-1a), V₂ is CH. In certain embodiments of formula (y-1a), V₂ is CR^{x}.

As generally defined herein, V₃ is independently O, S, N, NH, NR^{x}, CH, or CR^{x}, as valency permits. In certain embodiments of formula (y-1a), V₃ is N. In certain of formula (y-1a), V₃ is CH. In certain embodiments of formula (y-1a), V₃ is CR^{x}.

In certain embodiments of formula (y-1a), V₁ is N and V₂ is CR^{x}. In certain embodiments of formula (y-1a), V₁ is N and V₂ is CH. In certain embodiments of formula (y-1a), V₁ is N and V₂ is N. In certain embodiments of formula (y-1a), V₁ is N and V₃ is CR^{x}. In certain embodiments of formula (y-1a), V₁ is N and V₃ is CH. In certain embodiments of formula (y-1a), V₁ is N and V₃ is N. In certain embodiments of formula (y-1a), V₂ is N and V₃ is CR^{x}. In certain embodiments of formula (y-1a), V₂ is N and V₃ is CH. In certain embodiments of formula (y-1a), V₂ is N and V₃ is N.

In certain embodiments of formula (y-1a), V₁ is N; V₂ is CR^{x}; and V₃ is N, CH, or CR^{x}. In certain embodiments of formula (y-1a), V₁ is N; V₂ is CH; and V₃ is N, CH, or CR^{x}. In certain embodiments of formula (y-1a), V₁ is N; V₂ is N; and V₃ is N, CH, or CR^{x}. In certain embodiments of formula (y-1a), V₁ is N; V₃ is CR^{x}; and V₂ is N, CH, or CR^{x}. In certain embodiments of formula (y-1a), V₁ is N; V₃ is CH; and V₂ is N, CH, or CR^{x}. In certain embodiments of formula (y-1a), V₁ is N; V₃ is N; and V₂ is N, CH, or CR^{x}. In certain embodiments of formula (y-1a), V₂ is N; V₃ is CR^{x}; and V₁ is N, CH, or CR^{x}. In certain embodiments of formula (y-1a), V₂ is N; V₃ is CH; and V₁ is N, CH, or CR^{x}. In certain embodiments of formula (y-1a), V₂ is N; V₃ is N; and V₁ is N, CH, or CR^{x}.

Exemplary compounds of formula (y-1a), include, but are not limited to:

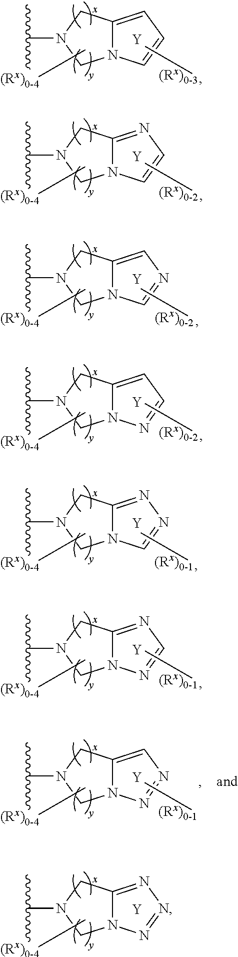

wherein the dihydro-imidazolyl ring of the bicyclic ring system comprises 0, 1, 2, 3, or 4 R^{x} substituents, and Ring Y of the bicyclic ring system comprises 0, 1, 2, or 3 R^{x} substituents.

In certain embodiments of formula (y-1), V₄ is C and Y¹ is a bicyclic ring system of formula (y-1b):

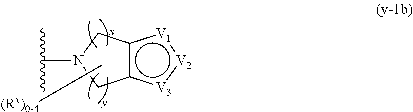

wherein V₄, V₅, x, y, R^{x}, V₁, V₂, and V₃ are as defined herein.

In certain embodiments of formula (y-1b), Y¹ is of formula (y-1b-i), (y-1b-ii), or (y-1b-iii),

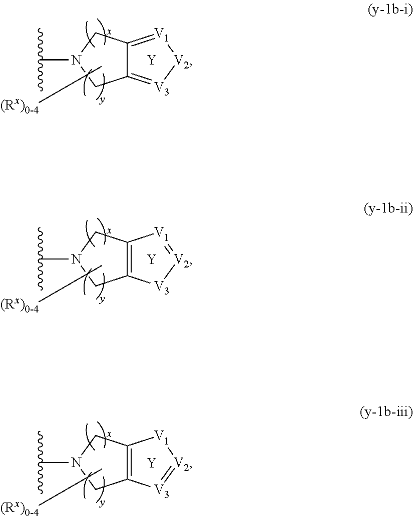

wherein each instance of V₁ and V₃ is independently N, CH, or CR^{x}, and V₂ is O, S, NH, or NR^{x}, wherein R^{x} is as defined herein.

In certain embodiments of formula (y-1b-i), (y-1b-ii), or (y-1b-iii), V₁ is N. In certain embodiments of formula (y-1b-i), (y-1b-ii), or (y-1b-iii), V₁ is CH. In certain embodiments of formula (y-1b-i), (y-1b-ii), or (y-1b-iii), V₁ is CR^{x}. In certain embodiments of formula (y-1b-i), (y-1b-ii), or (y-1b-iii), V₃ is N. In certain embodiments of formula (y-1b-i), (y-1b-ii), or (y-1b-iii), V₃ is CH. In certain embodiments of formula (y-1b-i), (y-1b-ii), or (y-1b-iii), V₃ is CR^{x}. In certain embodiments of formula (y-1b-i), (y-1b-ii), or (y-1b-iii), V₂ is O. In certain embodiments of formula (y-1b-i), (y-1b-ii), or (y-1b-iii), V₂ is S. In certain embodiments of formula (y-1b-i), (y-1b-ii), or (y-1b-iii), V₂ is NH. In certain embodiments of formula (y-1b-i), (y-1b-ii), or (y-1b-iii), V₂ is NR^{x}.

In certain embodiments of formula (y-1b-i), (y-1b-ii), or (y-1b-iii), V₁ is N and V₂ is O. In certain embodiments, V₁ is CH or CR^{x} and V₂ is O. In certain embodiments, V₁ is N and V₂ is S. In certain embodiments, V₁ is CH or CR^{x} and V₂ is S. In certain embodiments, V₁ is N and V₂ is NH or NR^{x}. In certain embodiments, V₁ is CH or CR^{x} and V₂ is NH or NR^{x}. In certain embodiments, V₁ is N and V₃ is N. In certain embodiments, V₁ is CH or CR^{x} and V₃ is N. In certain embodiments, V₁ is N and V₃ is CH or CR^{x}. In certain embodiments, V₁ is CH or CR^{x} and V₃ is CH or CR^{x}. In certain embodiments, V₃ is N and V₂ is O. In certain embodiments, V₃ is CH or CR^{x} and V₂ is O. In certain embodiments, V₃ is N and V₂ is S. In certain embodiments, V₃ is CH or CR^{x} and V₂ is S. In certain embodiments, V₃ is N and V₂ is NH or NR^{x}. In certain embodiments, V₃ is CH or CR^{x} and V₂ is NH or NR^{x}.

In certain embodiments of formula (y-1), Ring Y is unsubstituted (i.e., does not comprise an R^{x} substituent). However, in certain embodiments, Ring Y is substituted with at least one R^{x} group. In certain embodiments, Ring Y is substituted with two R^{x} groups. In certain embodiments, Ring Y is substituted with three R^{x} groups.

Exemplary ring systems of formula (y-1) include, but are not limited to:

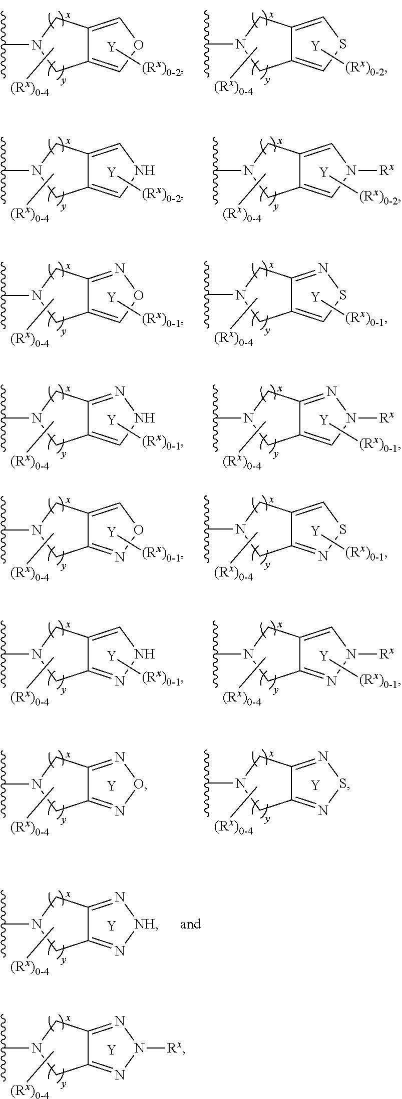

wherein the ring at the point of attachment comprises 0, 1, 2, 3, or 4 R^{x} substituents, and Ring Y comprises 0, 1, or 2 R^{x} substituents.

Exemplary ring systems of formula (y-1) include, but are not limited to:

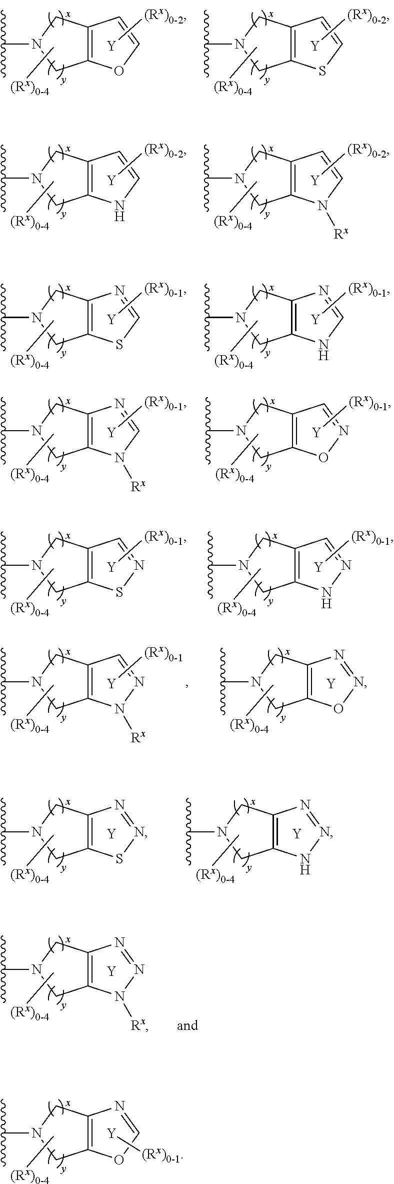

wherein the ring at the point of attachment comprises 0, 1, 2, 3, or 4 R^{x} substituents, and Ring Y comprises 0, 1, or 2 R^{x} substituents.

Exemplary ring systems of formula (y-1) include, but are not limited to:

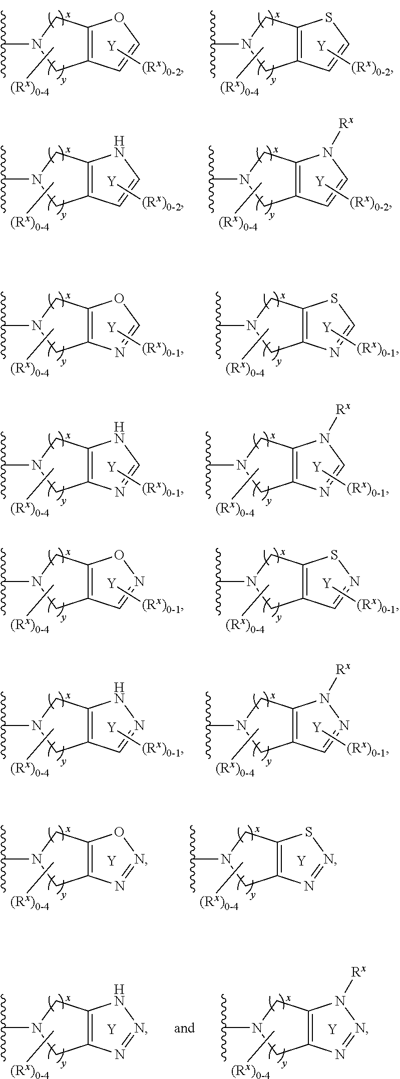

wherein the ring at the point of attachment comprises 0, 1, 2, 3, or 4 R^{x} substituents, and Ring Y comprises 0, 1, or 2 R^{x} substituents.

In certain embodiments of Formula (A), Y¹ is a bicyclic ring system of formula (y-1c):

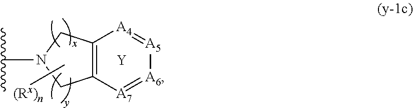

wherein each instance of A₄, A₅, A₆, and A₇ is independently N, CH, or CR^{x}, provided at least one of A₄, A₅, A₆, and A₇ is N, and wherein R^{x} is as defined herein.

In certain embodiments of formula (y-1c), one of A₄, A₅, A₆, and A₇ is N. In certain embodiments, two of A₄, A₅, A₆, and A₇ is N.

In certain embodiments of formula (y-1c), A₄ is N. In certain embodiments, A₄ is CH. In certain embodiments, A₄ is CR^{x}. In certain embodiments, A₅ is N. In certain embodiments, A₅ is CH. In certain embodiments, A₅ is CR^{x}. In certain embodiments, A₆ is N. In certain embodiments, A₆ is CH. In certain embodiments, A₆ is CR^{x}. In certain embodiments, A₇ is N. In certain embodiments, A₇ is CH. In certain embodiments, A₇ is CR^{x}.

In certain embodiments of formula (y-1c), A₄ is N, and A₅, A₆, and A₇ are each independently CH or CR^{x}. In certain embodiments, A₅ is N, and A₄, A₆, and A₇ are each independently CH or CR^{x}. In certain embodiments, A₆ is N, and A₄, A₅, and A₇ are each independently CH or CR^{x}. In certain embodiments, A₇ is N, and A₄, A₅, and A₆ are each independently CH or CR^{x}. In certain embodiments, each of A₄ and A₅ is N, and A₆ and A₇ are each independently CH or CR^{x}. In certain embodiments, each of A₄ and A₆ is N, and A₅ and A₇ are each independently CH or CR^{x}. In certain embodiments, each of A₄ and A₇ is N, and A₅ and A₆ are each independently CH or CR^{x}. In certain embodiments, each of A₅ and A₆ is N, and A₄ and A₇ are each independently CH or CR^{x}. In certain embodiments, each of A₆ and A₇ is N, and A₄ and A₅ are each independently CH or CR^{x}. In certain embodiments, each of A₅ and A₇ is N, and A₄ and A₆ are each independently CH or CR^{x}.

In certain embodiments of formula (y-1c), Ring Y is unsubstituted (i.e., does not comprise an R^{x} substituent). However, in certain embodiments, Ring Y is substituted with at least one R^{x} group. In certain embodiments, Ring Y is substituted with two R^{x} groups. In certain embodiments, Ring Y is substituted with three R^{x} groups.

Exemplary ring systems of formula (y-1c) include, but are not limited to:

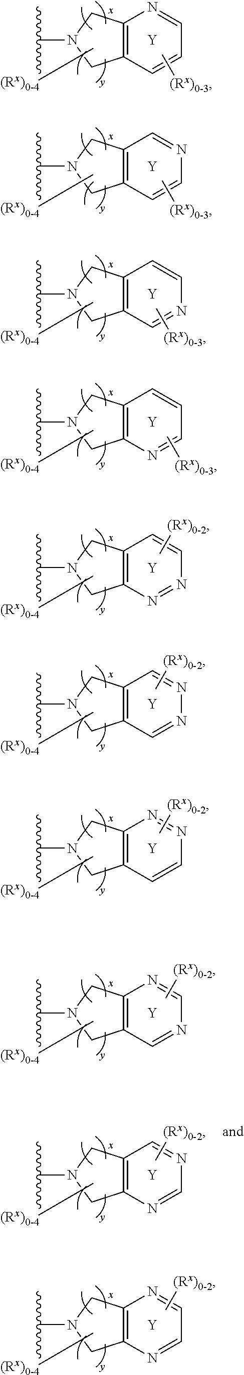

wherein the ring at the point of attachment comprises 0, 1, 2, 3, or 4 R^{x} substituents, and Ring Y comprises 0, 1, 2, or 3 R^{x} substituents.

As defined generally above, R¹ is hydrogen, R^{z}, or —C(O)R^{z}, wherein R^{z} is optionally substituted C₁₋₆ alkyl. In certain embodiments, R¹ is hydrogen. In some embodiments, R¹ is optionally substituted C₁₋₆ alkyl. In certain embodiments, R¹ is unsubstituted C₁₋₆ alkyl. In certain embodiments, R¹ is methyl, ethyl, or propyl. In some embodiments, R¹ is —C(O)R^{z}, wherein R^{z} is optionally substituted C₁₋₆ alkyl. In certain embodiments, R¹ is —C(O)R^{z}, wherein R^{z} is unsubstituted C₁₋₆ alkyl. In certain embodiments, R¹ is acetyl.

As defined generally above, L_{z} is a linker or is absent. In certain embodiments, L_{z} is —X_{A}—C(R^{2A})(R^{3A})C(═O)N(R)—, L_{B}, or L_{D} as described herein.

As defined generally above, Ring Z is an optionally substituted, monocyclic or bicyclic, saturated, partially unsaturated, or aromatic ring having 0-4 heteroatoms independently selected from nitrogen, oxygen, and sulfur. In certain embodiments, Ring Z is Ring A, Ring C, Cy^{A}, or Ar as described herein.

In certain embodiments, Ring Z is not a phenyl ring monosubstituted by optionally substituted 5,6-bicyclic heteroaryl or optionally substituted 5,5,-bicyclic heteroaryl. In certain embodiments, Ring Z is not a phenyl ring monosubstituted by 5,6-bicyclic heteroaryl or 5,5,-bicyclic heteroaryl. In certain embodiments, Ring Z is not a phenyl ring monosubstituted by 5,6-bicyclic heteroaryl or 5,5,-bicyclic heteroaryl, wherein the 5,6-bicyclic heteroaryl or 5,5,-bicyclic heteroaryl has an isoxazole ring. In certain embodiments, Ring Z is not a phenyl ring monosubstituted by 5,6-bicyclic heteroaryl or 5,5,-bicyclic heteroaryl, wherein the 5,6-bicyclic heteroaryl or 5,5,-bicyclic heteroaryl is an isoxazole ring fused to a monosubstituted phenyl ring or a thiophene ring. In certain embodiments, Ring Z is not

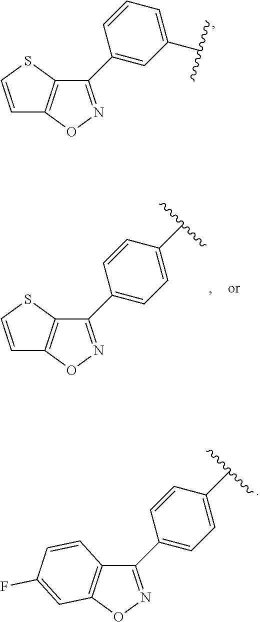 [SMILES: *c1cccc(-c2noc3ccsc23)c1 ; *c1ccc(-c2noc3ccsc23)cc1 ; *c1ccc(-c2noc3cc(F)ccc23)cc1]

As defined generally above, X_{A} is a bond, —O—, —N(R)—, —CR^{4A}R^{5A}—, —O—CR^{4A}R^{5A}—N(R)—CR^{4A}R^{5A}—, —O—CR^{4A}R^{5A}—O—, —N(R)—CR^{4A}R^{5A}—O, —N(R)—CR^{4A}R^{5A}—N(R)—, —O—CR^{4A}R^{5A}—N(R)—, —CR^{4A}R^{5A}—O—, —CR^{4A}R^{5A}—N(R)—, —O—CR^{4A}R^{5A}—CR^{6A}R^{7A}—, —N(R)—CR^{4A}R^{5A}—CR^{6A}R^{7A}—, —CR^{6A}R^{7A}—CR^{4A}R^{5A}—O—, —CR^{6A}R^{7A}—CR^{4A}R^{5A}—N(R)—, or —CR^{6A}R^{7A}—CR^{4A}R^{5A}—. In certain embodiments, X_{A} is a bond, —O—, —N(R)—, or —CR⁴R⁵—, wherein R, R⁴, and R⁵ are as described herein. In certain embodiments, X_{A} is a bond. In certain embodiments, X_{A} is —O—. In some embodiments, X_{A} is —N(R)—. In certain embodiments, X_{A} is —NH—. In certain embodiments, X_{A} is —N(R)—, wherein R is optionally substituted C₁₋₆ aliphatic. In certain embodiments, X_{A} is —N(R)—, wherein R is optionally substituted C₁₋₆ alkyl. In certain embodiments, X_{A} is —N(R)—, wherein R is unsubstituted C₁₋₆ alkyl. In certain embodiments, X_{A} is —N(Me)-. In some embodiments, X_{A} is —CR^{4A}R^{5A}—. In certain embodiments, X_{A} is —CH₂—. In certain embodiments, X_{A} is —CH₂—O—.

As defined generally above, each R is independently hydrogen or optionally substituted C₁₋₆ aliphatic. In certain embodiments, R is hydrogen. In some embodiments, R is optionally substituted C₁₋₆ aliphatic. In some embodiments, R is substituted C₁₋₆ aliphatic. In some embodiments, R is unsubstituted C₁₋₆ aliphatic. In some embodiments, R is optionally substituted C₁₋₆ alkyl. In some embodiments, R is substituted C₁₋₆ alkyl. In some embodiments, R is unsubstituted C₁₋₆ alkyl. In some embodiments, R is methyl, ethyl, or propyl.

As defined generally above, R^{2A} and R^{3A} are independently selected from the group consisting of hydrogen, halo, —CN, —NO₂, optionally substituted aliphatic, optionally substituted carbocyclyl, optionally substituted phenyl, optionally substituted heterocyclyl, optionally substituted heteroaryl, —OR^{A}, —N(R^{B})₂, —SR^{A}, —C(═O)R^{A}, —C(O)OR^{A}, —C(O)SR^{A}, —C(O)N(R^{B})₂, —C(O)N(R^{B})N(R^{B})₂, —OC(O)R^{A}, —OC(O)N(R^{B})₂, —NR^{B}C(O)R^{A}, —NR^{B}C(O)N(R^{B})₂, —NR^{B}C(O)N(R^{B})N(R^{B})₂, —NR^{B}C(O)OR^{A}, —SC(O)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(O)R^{A}, —OS(O)₂R^{A}, —SO₂R^{A}, —NR^{B}SO₂R^{A}, or —SO₂N(R^{B})₂; or R^{2A} and R^{3A} are taken together with their intervening atoms to form an optionally substituted carbocyclic or heterocyclic ring.

In certain embodiments, R^{2A} and R^{3A} are independently selected from the group consisting of hydrogen, halo, —CN, —NO₂, optionally substituted aliphatic, optionally substituted carbocyclyl, optionally substituted phenyl, optionally substituted heterocyclyl, optionally substituted heteroaryl, —OR^{A}, —N(R^{B})₂, —SR^{A}, —C(═O)R^{A}, —C(O)OR^{A}, —C(O)SR^{A}, —C(O)N(R^{B})₂, —OC(O)R^{A}, —NR^{B}C(O)R^{A}, —NR^{B}C(O)N(R^{B})₂, —SC(O)R^{A}, —C(═NR^{B})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(O)R^{A}, —SO₂R^{A}, —NR^{B}SO₂R^{A}, and —SO₂N(R^{B})₂; or R^{2A} and R^{3A} are taken together with their intervening atoms to form an optionally substituted carbocyclic or heterocyclic ring.

In certain embodiments, R^{2A} is hydrogen. In some embodiments, R^{2A} is not hydrogen. In some embodiments, R^{2A} is halo. In certain embodiments, R^{2A} is fluoro. In some embodiments, R^{2A} is optionally substituted aliphatic. In certain embodiments, R^{2A} is optionally substituted C₁₋₆ aliphatic. In certain embodiments, R^{2A} is optionally substituted C₁₋₆ alkyl. In certain embodiments, R^{2A} is substituted C₁₋₆ alkyl. In certain embodiments, R^{2A} is —CF₃, CHF₂, or CH₂F. In certain embodiments, R^{2A} is unsubstituted C₁₋₆ alkyl. In certain embodiments, R^{2A} is methyl, ethyl, or propyl. In certain embodiments, R^{3A} is hydrogen. In some embodiments, R^{3A} is not hydrogen. In some embodiments, R^{3A} is halo. In certain embodiments, R^{3A} is fluoro.

In some embodiments, R^{3A} is optionally substituted aliphatic. In certain embodiments, R³ is optionally substituted C₁₋₆ aliphatic. In certain embodiments, R^{3A} is optionally substituted C₁₋₆ alkyl. In certain embodiments, R^{3A} is substituted C₁₋₆ alkyl. In certain embodiments, R^{3A} is —CF₃, CHF₂, or CH₂F. In certain embodiments, R^{3A} is unsubstituted C₁₋₆ alkyl. In certain embodiments, R^{3A} is methyl, ethyl, or propyl.

In some embodiments, R^{2A} and R^{3A} are the same. In some embodiments, R^{2A} and R^{3A} are different. In some embodiments, R^{2A} and R^{3A} are each hydrogen. In some embodiments, R^{2A} is hydrogen and R^{3A} is not hydrogen. In some embodiments, R^{2A} is hydrogen and R^{3A} is optionally substituted aliphatic. In some embodiments, R^{2A} is hydrogen and R^{3A} is C₁₋₆ alkyl. In some embodiments, R^{2A} is hydrogen and R^{3A} is methyl.

As defined generally above, R^{4A} and R^{5A} are independently selected from the group consisting of hydrogen, halo, —CN, —NO₂, optionally substituted aliphatic, optionally substituted carbocyclyl, optionally substituted phenyl, optionally substituted heterocyclyl, optionally substituted heteroaryl, —OR^{A}, —N(R^{B})₂, —SR^{A}, —C(═O)R^{A}, —C(O)OR^{A}, —C(O)SR^{A}, —C(O)N(R^{B})₂, —OC(O)R^{A}, —NR^{B}C(O)R^{A}, —NR^{B}C(O)N(R^{B})₂, —SC(O)R^{A}, —C(═NR^{B})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(O)R^{A}, —SO₂R^{A}, —NR^{B}SO₂R^{A}, and —SO₂N(R^{B})₂; or R^{4A} and R^{5A} are taken together with their intervening atoms to form an optionally substituted carbocyclic or heterocyclic ring.

In certain embodiments, R^{4A} and R^{5A} are independently selected from the group consisting of hydrogen, halo, —CN, —NO₂, optionally substituted aliphatic, optionally substituted carbocyclyl, optionally substituted phenyl, optionally substituted heterocyclyl, optionally substituted heteroaryl, —OR^{A}, —N(R^{B})₂, —SR^{A}, —C(═O)R^{A}, —C(O)OR^{A}, —C(O)SR^{A}, —C(O)N(R^{B})₂, —OC(O)R^{A}, —NR^{B}C(O)R^{A}, —NR^{B}C(O)N(R^{B})₂, —SC(O)R^{A}, —C(═NR^{B})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(O)R^{A}, —SO₂R^{A}, —NR^{B}SO₂R^{A}, and —SO₂N(R^{B})₂; or R^{4A} and R^{5A} are taken together with their intervening atoms to form an optionally substituted carbocyclic or heterocyclic ring.

In certain embodiments, R^{4A} is hydrogen. In some embodiments, R^{4A} is not hydrogen. In some embodiments, R^{4A} is halo. In certain embodiments, R^{4A} is fluoro. In some embodiments, R^{4A} is optionally substituted aliphatic. In certain embodiments, R^{4A} is optionally substituted C₁₋₆ aliphatic. In certain embodiments, R^{4A} is optionally substituted C₁₋₆ alkyl. In certain embodiments, R^{4A} is substituted C₁₋₆ alkyl. In certain embodiments, R^{4A} is —CF₃, CHF₂, or CH₂F. In certain embodiments, R^{4A} is unsubstituted C₁₋₆ alkyl. In certain embodiments, R^{4A} is methyl, ethyl, or propyl.

In certain embodiments, R^{5A} is hydrogen. In some embodiments, R^{5A} is not hydrogen. In some embodiments, R^{5A} is halo. In certain embodiments, R^{5A} is fluoro. In some embodiments, R^{5A} is optionally substituted aliphatic. In certain embodiments, R^{5A} is optionally substituted C₁₋₆ aliphatic. In certain embodiments, R^{5A} is optionally substituted C₁₋₆ alkyl. In certain embodiments, R^{5A} is substituted C₁₋₆ alkyl. In certain embodiments, R^{5A} is —CF₃, CHF₂, or CH₂F. In certain embodiments, R^{5A} is unsubstituted C₁₋₆ alkyl. In certain embodiments, R^{5A} is methyl, ethyl, or propyl.

In some embodiments, R^{4A} and R^{5A} are the same. In some embodiments, R^{4A} and R^{5A} are different. In some embodiments, R^{4A} and R^{5A} are each hydrogen. In some embodiments, R^{4A} is hydrogen and R^{5A} is not hydrogen. In some embodiments, R^{4A} is hydrogen and R^{5A} is optionally substituted aliphatic. In some embodiments, R^{4A} is hydrogen and R^{5A} is C₁₋₆ alkyl. In some embodiments, R^{4A} is hydrogen and R^{5A} is methyl.

As defined generally above, R^{6A} and R^{7A} are independently selected from the group consisting of hydrogen, halo, —CN, —NO₂, optionally substituted aliphatic, optionally substituted carbocyclyl, optionally substituted phenyl, optionally substituted heterocyclyl, optionally substituted heteroaryl, —OR^{A}, —N(R^{B})₂, —SR^{A}, —C(═O)R^{A}, —C(O)OR^{A}, —C(O)SR^{A}, —C(O)N(R^{B})₂, —C(O)N(R^{B})N(R^{B})₂, —OC(O)R^{A}, —OC(O)N(R^{B})₂, —NR^{B}C(O)R^{A}, —NR^{B}C(O)N(R^{B})₂, —NR^{B}C(O)N(R^{B})N(R^{B})₂, —NR^{B}C(O)OR^{A}, —SC(O)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(O)R^{A}, —OS(O)₂R^{A}, —SO₂R^{A}, —NR^{B}SO₂R^{A}, or —SO₂N(R^{B})₂; or R^{6A} and R^{7A} are taken together with their intervening atoms to form an optionally substituted carbocyclic or heterocyclic ring.

In certain embodiments, R^{6A} and R^{7A} are independently selected from the group consisting of hydrogen, halo, —CN, —NO₂, optionally substituted aliphatic, optionally substituted carbocyclyl, optionally substituted phenyl, optionally substituted heterocyclyl, optionally substituted heteroaryl, —OR^{A}, —N(R^{B})₂, —SR^{A}, —C(═O)R^{A}, —C(O)OR^{A}, —C(O)SR^{A}, —C(O)N(R^{B})₂, —OC(O)R^{A}, —NR^{B}C(O)R^{A}, —NR^{B}C(O)N(R^{B})₂, —SC(O)R^{A}, —C(═NR^{B})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(O)R^{A}, —SO₂R^{A}, —NR^{B}SO₂R^{A}, and —SO₂N(R^{B})₂; or R^{6A} and R^{7A} are taken together with their intervening atoms to form an optionally substituted carbocyclic or heterocyclic ring.

In certain embodiments, R^{6A} is hydrogen. In some embodiments, R^{6A} is not hydrogen. In some embodiments, R^{6A} is halo. In certain embodiments, R^{6A} is fluoro. In some embodiments, R^{6A} is optionally substituted aliphatic. In certain embodiments, R^{6A} is optionally substituted C₁₋₆ aliphatic. In certain embodiments, R^{6A} is optionally substituted C₁₋₆ alkyl. In certain embodiments, R^{6A} is substituted C₁₋₆ alkyl. In certain embodiments, R^{6A} is —CF₃, CHF₂, or CH₂F. In certain embodiments, R^{6A} is unsubstituted C₁₋₆ alkyl. In certain embodiments, R^{6A} is methyl, ethyl, or propyl.

In certain embodiments, R^{7A} is hydrogen. In some embodiments, R^{7A} is not hydrogen. In some embodiments, R^{7A} is halo. In certain embodiments, R^{7A} is fluoro. In some embodiments, R^{7A} is optionally substituted aliphatic. In certain embodiments, R^{7A} is optionally substituted C₁₋₆ aliphatic. In certain embodiments, R^{7A} is optionally substituted C₁₋₆ alkyl. In certain embodiments, R^{7A} is substituted C₁₋₆ alkyl. In certain embodiments, R^{7A} is —CF₃, CHF₂, or CH₂F. In certain embodiments, R^{7A} is unsubstituted C₁₋₆ alkyl. In certain embodiments, R^{7A} is methyl, ethyl, or propyl.

In some embodiments, R^{6A} and R^{7A} are the same. In some embodiments, R^{6A} and R^{7A} are different. In some embodiments, R^{6A} and R^{7A} are each hydrogen. In some embodiments, R^{6A} is hydrogen and R^{7A} is not hydrogen. In some embodiments, R^{6A} is hydrogen and R^{7A} is optionally substituted aliphatic. In some embodiments, R^{6A} is hydrogen and R^{7A} is C₁₋₆ alkyl. In some embodiments, R^{6A} is hydrogen and R^{7A} is methyl.

As generally defined above, R¹² is hydrogen, halogen, or optionally substituted C₁₋₃alkyl. In certain embodiments, R¹² is hydrogen. In certain embodiments, R¹² is optionally substituted C₁₋₃alkyl, e.g., optionally substituted with halogen. In certain embodiments, R¹² is optionally substituted C₁alkyl, e.g., methyl or trifluoromethyl. In certain embodiments, R¹² is optionally substituted C₂ alkyl, e.g., ethyl. In certain embodiments, R¹² is optionally substituted C₃ alkyl, e.g., propyl. In certain embodiments, R¹² is fluoro, provided that R¹³ is not —OR¹. In certain embodiments, R¹² is chloro, provided that R¹³ is not —OR¹. In certain embodiments, R¹² is bromo, provided that R¹³ is not —OR¹. In certain embodiments, R¹² is iodo, provided that R¹³ is not —OR¹.

As generally defined above, R¹³ is hydrogen, halogen, optionally substituted C₁₋₃alkyl, or —OR¹. In certain embodiments, R¹³ is hydrogen. In certain embodiments, R¹³ is optionally substituted C₁₋₃alkyl, e.g., optionally substituted with halogen. In certain embodiments, R¹³ is optionally substituted C₁alkyl, e.g., methyl or trifluoromethyl. In certain embodiments, R¹³ is optionally substituted C₂ alkyl, e.g., ethyl. In certain embodiments, R¹³ is optionally substituted C₃ alkyl, e.g., propyl. In certain embodiments, R¹³ is fluoro. In certain embodiments, R¹³ is chloro. In certain embodiments, R¹³ is bromo. In certain embodiments, R¹³ is iodo.

As defined generally above, R²¹, R²², R²³, and R²⁴ are independently hydrogen, halo, or optionally substituted aliphatic. In some embodiments, R²¹, R²², R²³, and R²⁴ are hydrogen. In some embodiments, R²², R²³, and R²⁴ are hydrogen, and R²¹ is optionally substituted aliphatic. In some embodiments, R²², R²³, and R²⁴ are hydrogen, and R²¹ is optionally substituted C₁₋₆ aliphatic. In some embodiments, R²², R²³, and R²⁴ are hydrogen, and R²¹ is optionally substituted C₁₋₃ aliphatic. In some embodiments, R²², R²³, and R²⁴ are hydrogen, and R²¹ is methyl. In some embodiments, R²¹, R²², and R²³ are hydrogen, and R²⁴ is optionally substituted aliphatic. In some embodiments, R²¹, R²², and R²³ are hydrogen, and R²⁴ is optionally substituted C₁₋₆ aliphatic. In some embodiments, R²¹, R²², and R²³ are hydrogen, and R²⁴ is optionally substituted C₁₋₃ aliphatic. In some embodiments, R²¹, R²², and R²³ are hydrogen, and R²⁴ is methyl.

As defined generally above, L_{B} is —N(R)C(O)—, —C(O)N(R)—, —N(R)C(O)N(R)—, —N(R)C(O)O—, or —OC(O)N(R)—, wherein R is as described herein. In some embodiments, L_{B} is —N(R)C(O)—. In some embodiments, L_{B} is —NHC(O)—. In some embodiments, L_{B} is —N(C₁₋₆ alkyl)C(O)—. In some embodiments, L_{B} is —N(CH₃)C(O)—. In some embodiments, L_{B} is —C(O)N(R)—. In some embodiments, L_{B} is —C(O)NH—. In some embodiments, L_{B} is —C(O)N(C₁₋₆ alkyl)-. In some embodiments, L_{B} is —C(O)N(CH₃)—. In some embodiments, L_{B} is —N(R)C(O)N(R)—. In some embodiments, L_{B} is —NHC(O)NH—. In some embodiments, L_{B} is —NHC(O)N(R)—. In some embodiments, L_{B} is —N(R)C(O)NH—. In some embodiments, L_{B} is —N(CH₃)C(O)N(R)—. In some embodiments, L_{B} is —N(R)C(O)N(CH₃)—. In some embodiments, L_{B} is —N(CH₃)C(O)N(CH₃)—. In some embodiments, L_{B} is —N(R)C(O)O—. In some embodiments, L_{B} is —NHC(O)O—. In some embodiments, L_{B} is —N(C₁₋₆ alkyl)C(O)O—. In some embodiments, L_{B} is —N(CH₃)C(O)O—. In some embodiments, L_{B} is —OC(O)N(R)—. In some embodiments, L_{B} is —OC(O)NH—. In some embodiments, L_{B} is —OC(O)N(C₁₋₆ alkyl)-. In some embodiments, L_{B} is —OC(O)N(CH₃)—.

For avoidance of confusion, though Ar is sometimes used to denote the element argon, as used herein Ar denotes a monocyclic or bicyclic aromatic ring having 0-4 heteroatoms independently selected from nitrogen, oxygen, and sulfur, wherein Ar is substituted with 0, 1, 2, 3, 4, or 5 R^{y} groups, as valency permits, and various embodiments thereof as described herein, or Ar is a monocyclic or bicyclic heterocyclic ring having 1-4 heteroatoms independently selected from nitrogen, oxygen, and sulfur, wherein Ar is substituted with 0, 1, 2, 3, 4, or 5 R^{y} groups, as valency permits, and various embodiments thereof as described herein. In certain embodiments, Ar is unsubstituted. In certain embodiments, Ar is substituted with one or two R^{y} groups. In certain embodiments, Ar is substituted with one R^{y} group. In certain embodiments, Ar is substituted with two R^{y} groups. In certain embodiments, Ar is substituted with three R^{y} groups. In certain embodiments, Ar is substituted with four R^{y} groups. In certain embodiments, Ar is substituted with five R^{y} groups.

In certain embodiments, Ar is phenyl substituted with 0, 1, 2, 3, 4, or 5 R^{y} groups. In certain embodiments, Ar is phenyl substituted with one or two R^{y} groups. In certain embodiments, Ar is unsubstituted phenyl. In certain embodiments, Ar is phenyl substituted with one R^{y} group. In certain embodiments, Ar is phenyl substituted with two R^{y} groups. In certain embodiments, Ar is phenyl substituted with three R^{y} groups. In certain embodiments, Ar is phenyl substituted with four R^{y} groups. In certain embodiments, Ar is phenyl substituted with five R^{y} groups.

In certain embodiments, Ar is heteroaryl substituted with 0, 1, 2, 3, 4, or 5 R^{y} groups, as valency permits. In certain embodiments, Ar is a 5- to 6-membered heteroaryl having 1-3 heteroatoms independently selected from nitrogen, oxygen, and sulfur, and is substituted with 0, 1, 2, 3, or 4 R^{y} groups. In certain embodiments, Ar is an unsubstituted 5- to 6-membered heteroaryl having 1-3 heteroatoms independently selected from nitrogen, oxygen, and sulfur. In certain embodiments, Ar is a 5- to 6-membered heteroaryl having 1-3 heteroatoms independently selected from nitrogen, oxygen, and sulfur, and is substituted with one or two R^{y} groups. In certain embodiments, Ar is a 5- to 6-membered heteroaryl having 1-3 heteroatoms independently selected from nitrogen, oxygen, and sulfur, and is substituted with one R^{y} group. In certain embodiments, Ar is a 5-membered heteroaryl having 1-3 heteroatoms independently selected from nitrogen, oxygen, and sulfur (e.g., furanyl, thienyl, pyrrolyl, oxazolyl, isoxazolyl, thiazolyl, imidazolyl, pyrazolyl, isothiazolyl, triazolyl, oxadiazolyl, thiadiazolyl), and is substituted with 0, 1, 2, 3, or 4 R^{y} groups. In certain embodiments, Ar is a 6-membered heteroaryl having 1-3 nitrogens (e.g., pyridyl, pyrimidyl, pyridazinyl, pyrazinyl, triazinyl), and is substituted with 0, 1, 2, 3, 4, or 5 R^{y} groups.

In certain embodiments, Ar is a bicyclic aromatic ring having 0-4 heteroatoms independently selected from nitrogen, oxygen, and sulfur, wherein Ar is substituted with 0, 1, 2, 3, or 4 R^{y} groups. In certain embodiments, Ar is an 8- to 12-membered bicyclic aromatic ring having 0-4 heteroatoms independently selected from nitrogen, oxygen, and sulfur, wherein Ar is substituted with 0, 1, 2, 3, or 4 R^{y} groups. In certain embodiments, Ar is an unsubstituted bicyclic aromatic ring having 0-4 heteroatoms independently selected from nitrogen, oxygen, and sulfur. In certain embodiments, Ar is a bicyclic aromatic ring having 0-4 heteroatoms independently selected from nitrogen, oxygen, and sulfur, wherein Ar is substituted with one or two R^{y} groups. In certain embodiments, Ar is a bicyclic aromatic ring having 0-4 heteroatoms independently selected from nitrogen, oxygen, and sulfur, wherein Ar is substituted with one R^{y} group. In certain embodiments, Ar is a bicyclic aromatic ring having 0-4 heteroatoms independently selected from nitrogen, oxygen, and sulfur, wherein Ar is substituted with two R^{y} groups. In certain embodiments, Ar is a bicyclic aromatic ring having 0-4 heteroatoms independently selected from nitrogen, oxygen, and sulfur, wherein Ar is substituted with three R^{y} groups. In certain embodiments, Ar is a bicyclic aromatic ring having 0-4 heteroatoms independently selected from nitrogen, oxygen, and sulfur, wherein Ar is substituted with four R^{y} groups. In certain embodiments, Ar is a bicyclic aromatic ring having 0-4 heteroatoms independently selected from nitrogen, oxygen, and sulfur, wherein Ar is substituted with five R^{y} groups. In certain embodiments, Ar is naphthalene substituted with 0, 1, 2, 3, 4, or 5 R^{y} groups.

In certain embodiments, Ar is an 8- to 10-membered bicyclic heteroaryl having 1-4 heteroatoms independently selected from nitrogen, oxygen, and sulfur, wherein Ar is substituted with 0, 1, 2, 3, or 4 R^{y} groups. In certain embodiments, Ar is a 9-membered bicyclic heteroaryl having 1-3 heteroatoms independently selected from nitrogen, oxygen, and sulfur (e.g., indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl), wherein Ar is substituted with 0, 1, 2, 3, 4, or 5 R^{y} groups. In certain embodiments, Ar is a 10-membered bicyclic heteroaryl having 1-3 heteroatoms independently selected from nitrogen, oxygen, and sulfur (e.g., naphthyridinyl, quinolinyl, isoquinolinyl, quinoxalinyl, quinazolinyl), wherein Ar is substituted with 0, 1, 2, 3, 4, or 5 R^{y} groups. In certain embodiments, Ar is selected from the group consisting of quinoline, benzimidazole, benzopyrazole, quinoxaline, tetrahydroquinoline, tetrahydroisoquinoline, naphthalene, tetrahydronaphthalene, 2,3-dihydrobenzo[b][1,4]dioxine, isoindole, 2H-benzo[b][1,4]oxazin-3 (4H)-one, 3,4-dihydro-2H-benzo[b][1,4]oxazine, and quinoxalin-2(1H)-one, wherein Ar is substituted with 0, 1, 2, 3, or 4 R^{y} groups.

As generally defined above, in certain embodiments, Ar is a monocyclic or bicyclic heterocyclic ring having 1-4 heteroatoms independently selected from nitrogen, oxygen, and sulfur, wherein Ar is substituted with 0, 1, 2, 3, 4, or 5 R^{y} groups, as valency permits. In certain embodiments, Ar is a monocyclic heterocyclic ring, e.g., a monocyclic 5-membered or 6-membered heterocyclic ring substituted with 0, 1, 2, 3, 4, or 5 R^{y} groups, as valency permits. In certain embodiments, Ar is a bicyclic heterocyclic ring, e.g., a 6,6-bicyclic or 5,6-bicyclic heterocyclic ring substituted with 0, 1, 2, 3, 4, or 5 R^{y} groups, as valency permits. In certain embodiments, Ar is a 5,6-bicyclic heterocyclic ring wherein the point of attachment is on the 6-membered ring. In certain embodiments, wherein Ar is a 5,6-bicyclic heterocyclic ring, Ar is an optionally substituted dihydroimidazo pyrimidinyl ring.

In certain embodiments, Ring Z, e.g., Ar, Cy^{A}, Ring A, and the like, is selected from the group consisting of:

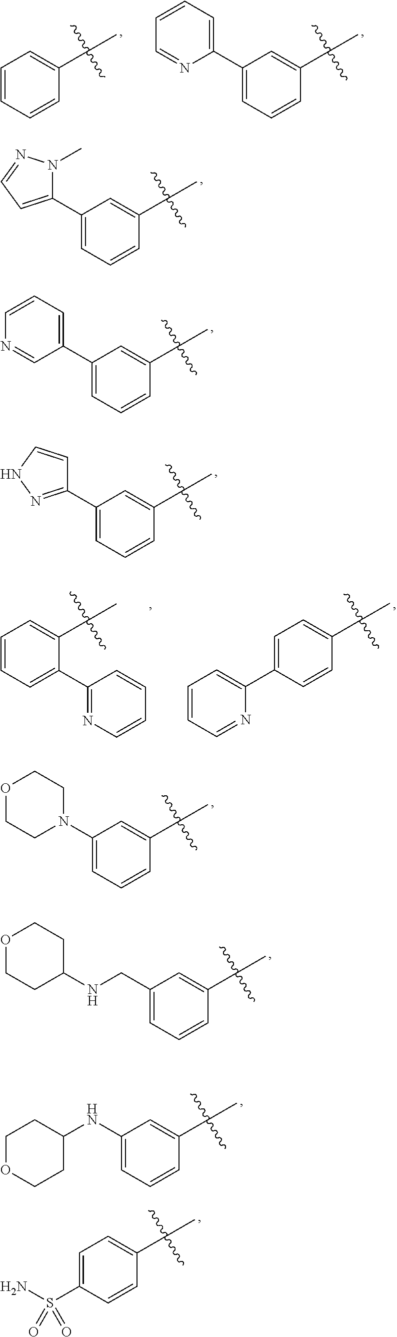

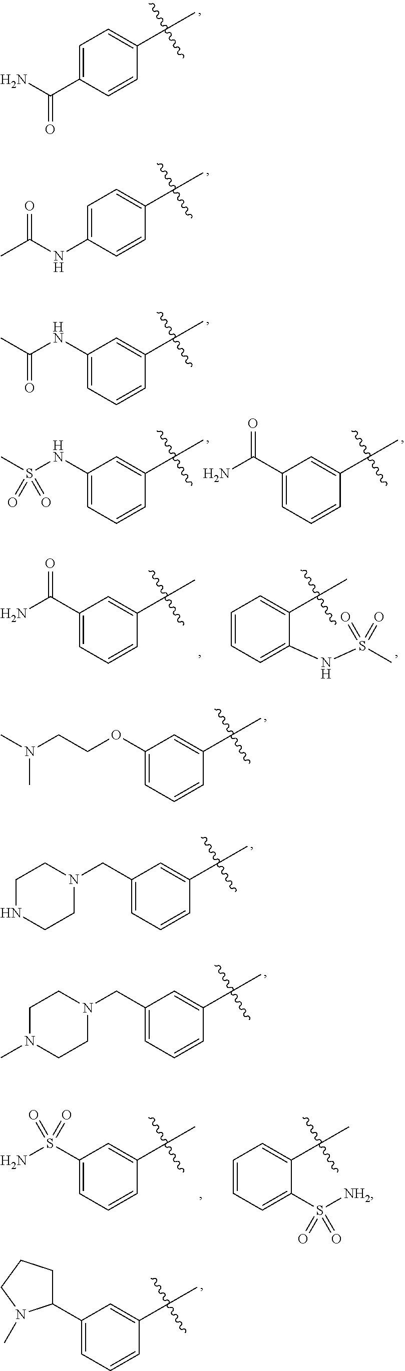

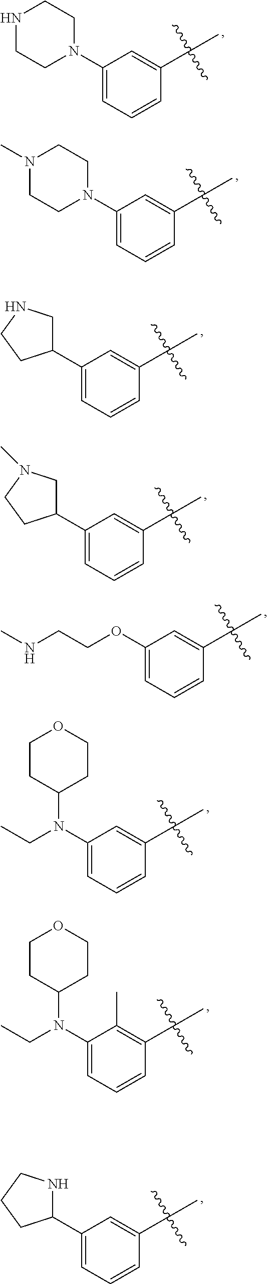

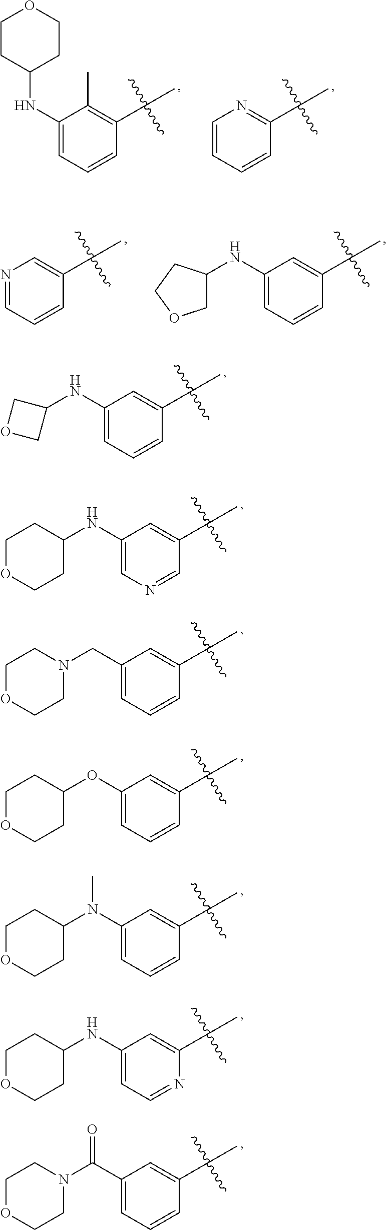

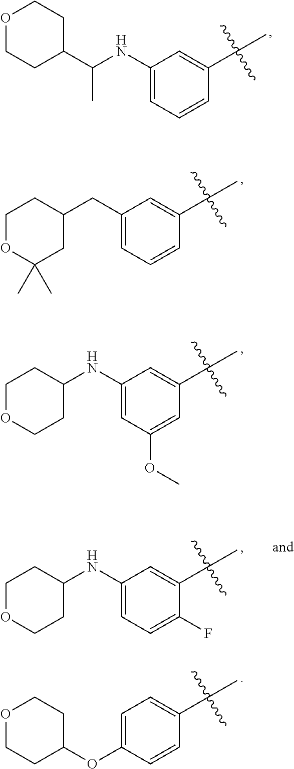 [SMILES: *c1cccc(NC(C)C2CCOCC2)c1 ; *c1cccc(CC2CCOC(C)(C)C2)c1 ; *c1cc(NC2CCOCC2)cc(OC)c1 ; *c1cc(NC2CCOCC2)ccc1F ; *c1ccc(OC2CCOCC2)cc1]

In certain embodiments, Ring Z, e.g., Ar, Cy^{A}, Ring A, and the like, is selected from the group consisting of:

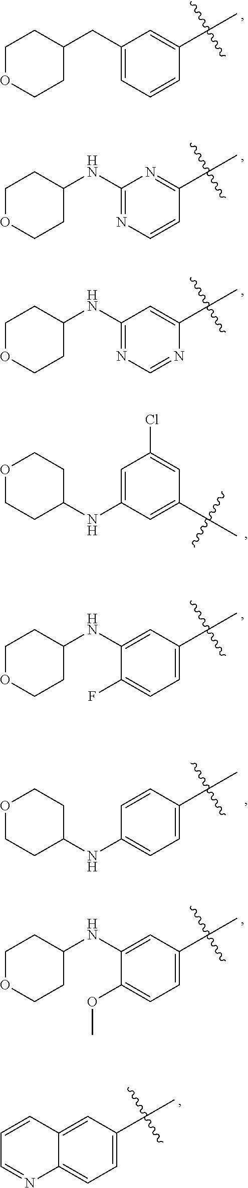

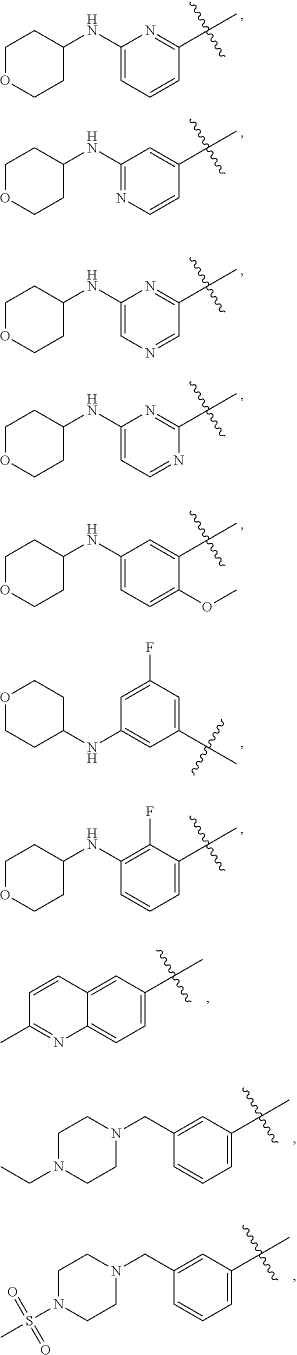

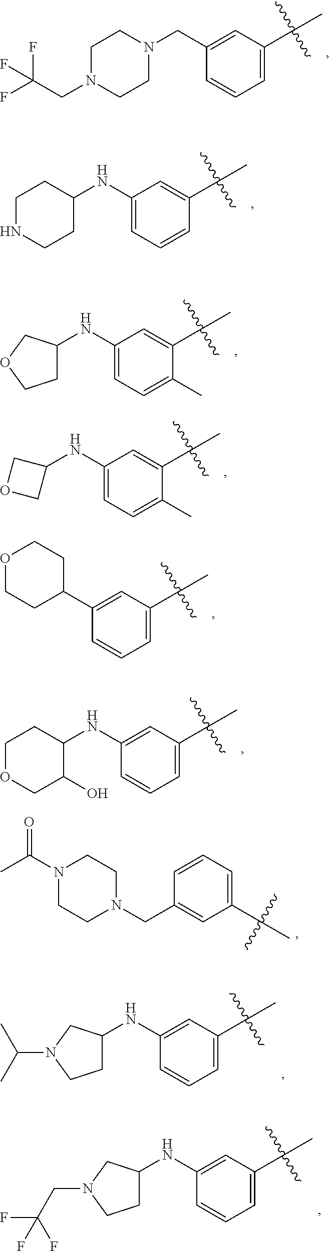

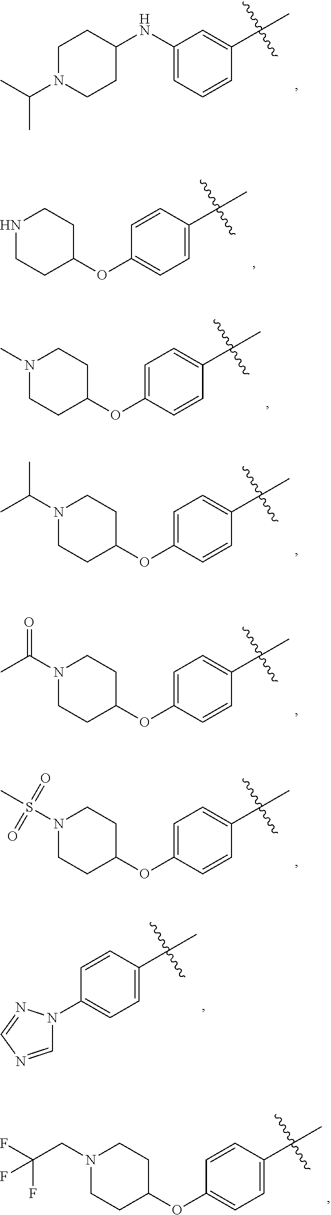

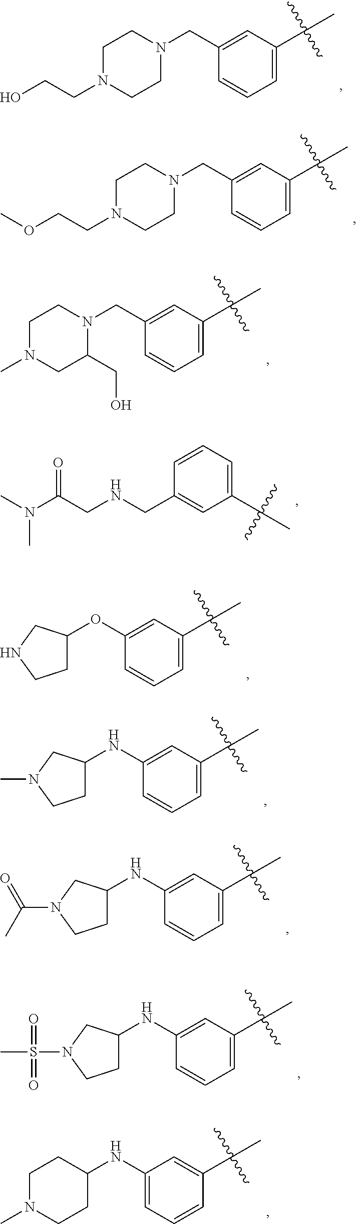

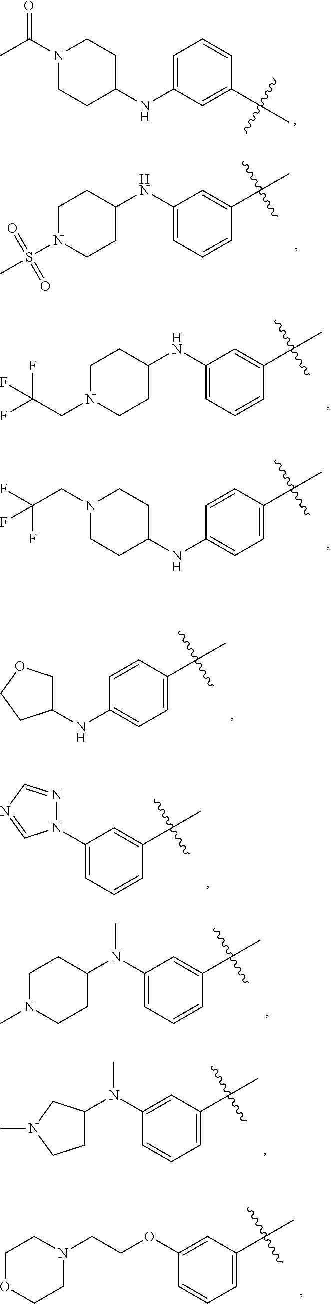

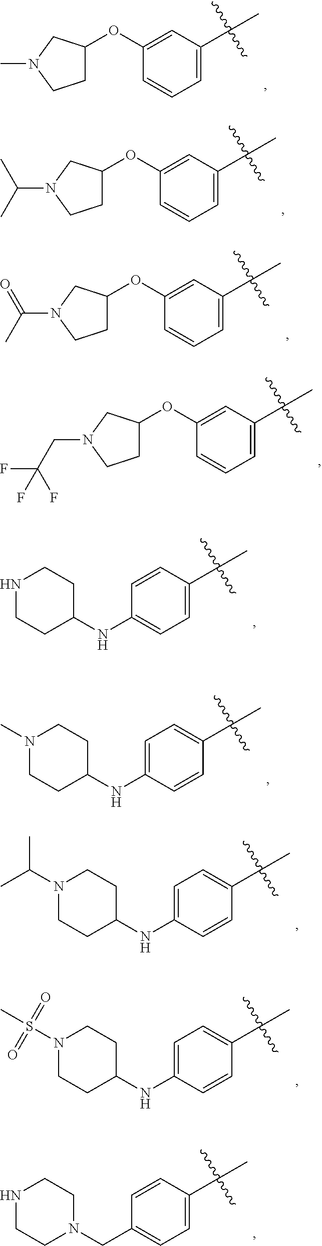

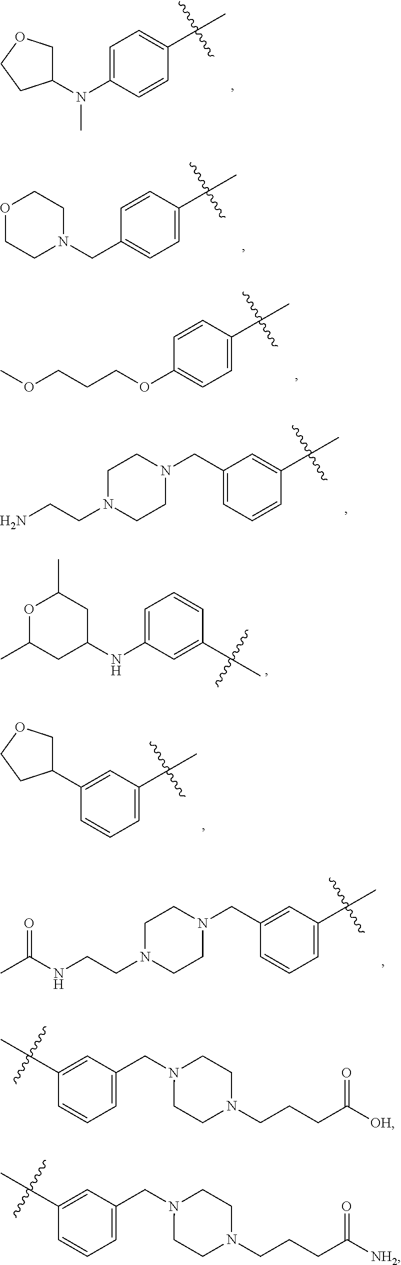

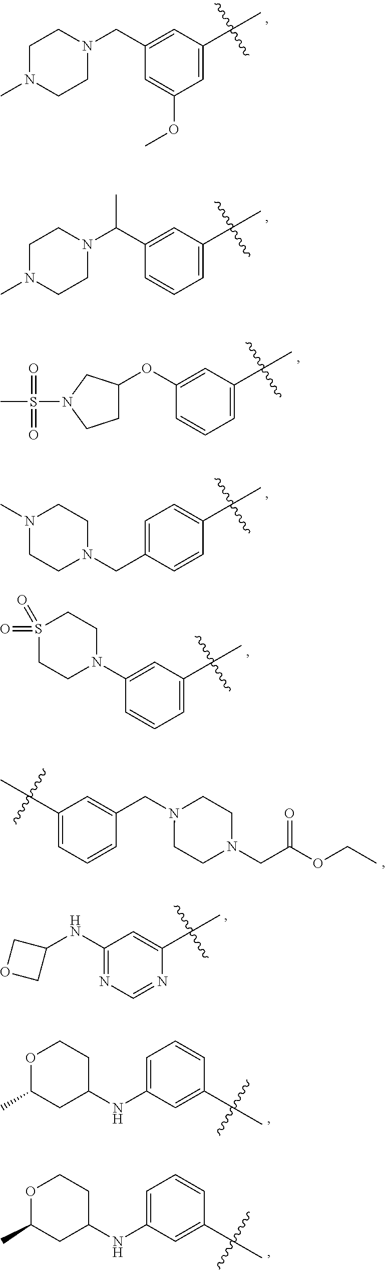

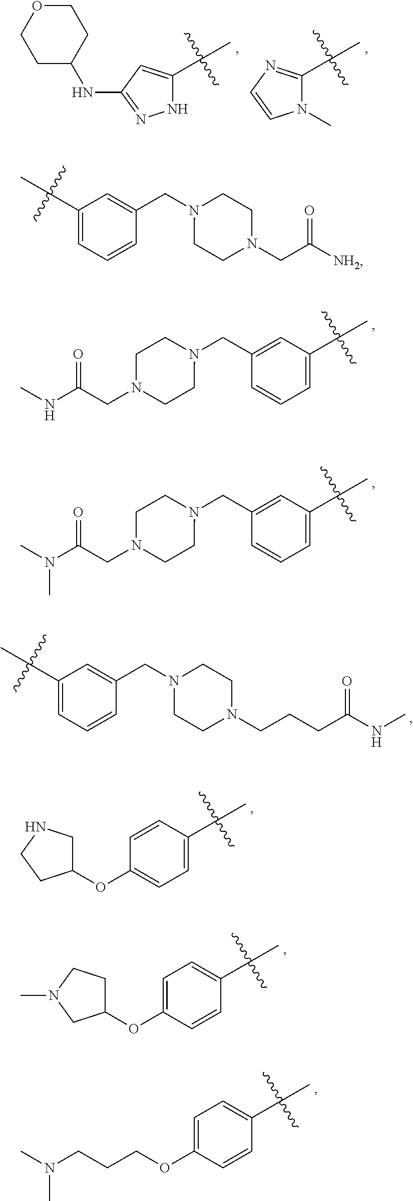

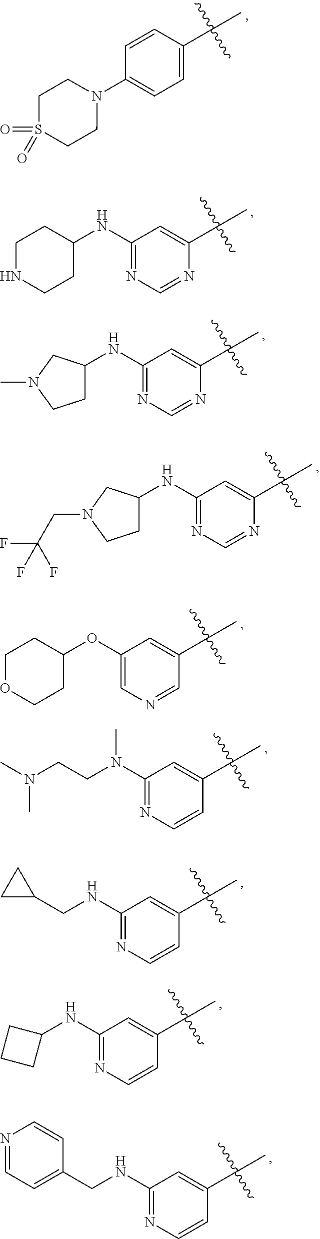

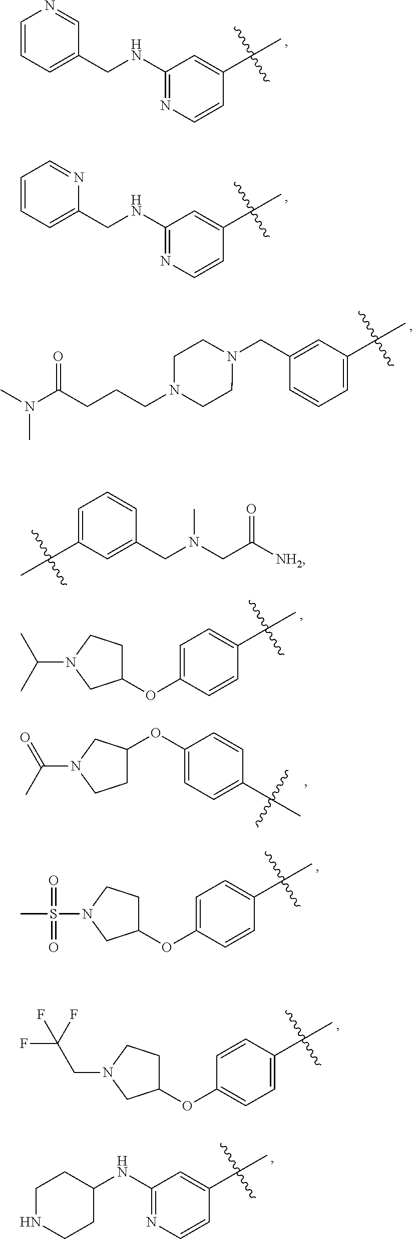

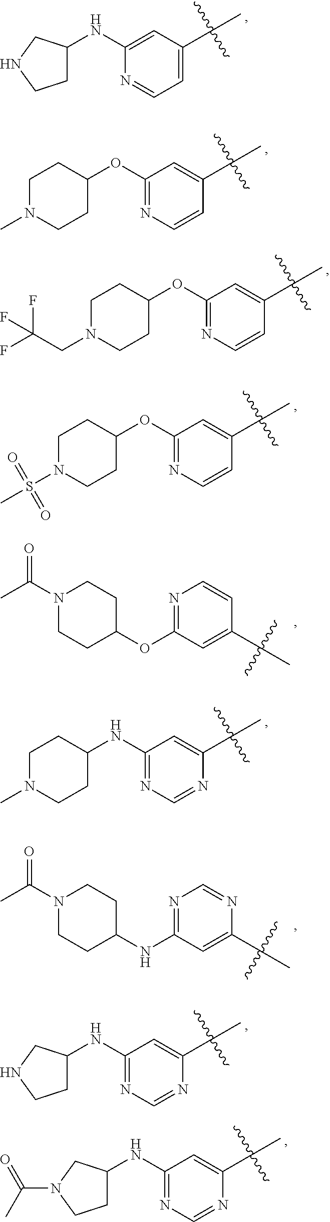

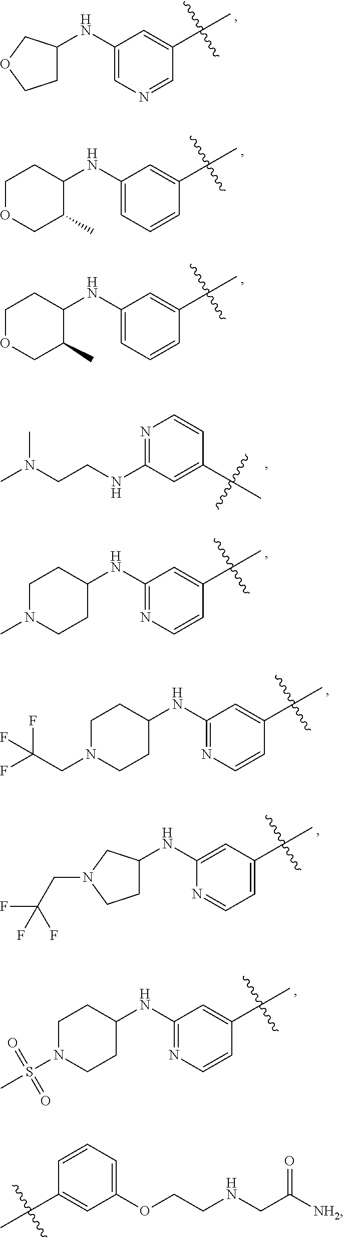

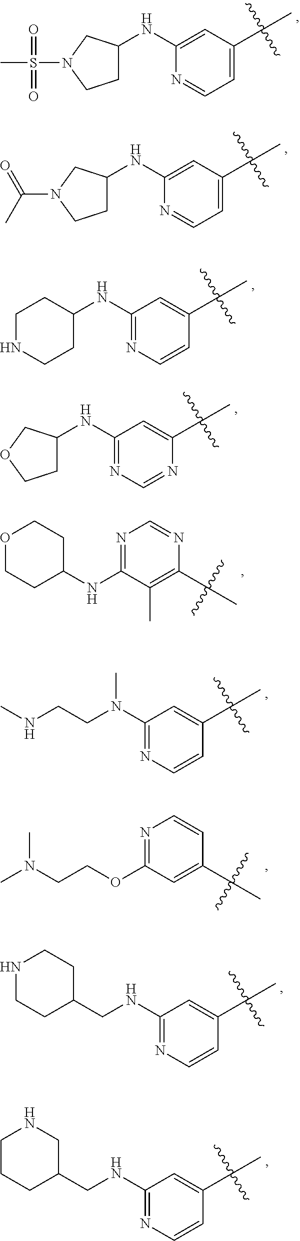

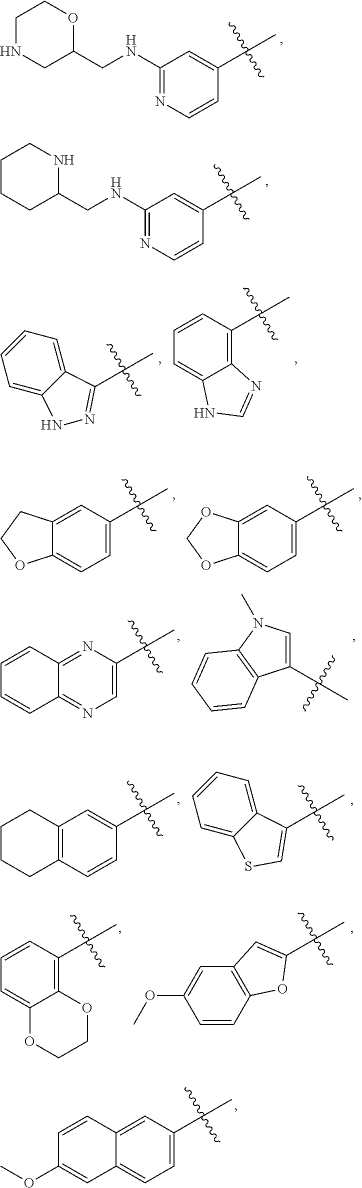

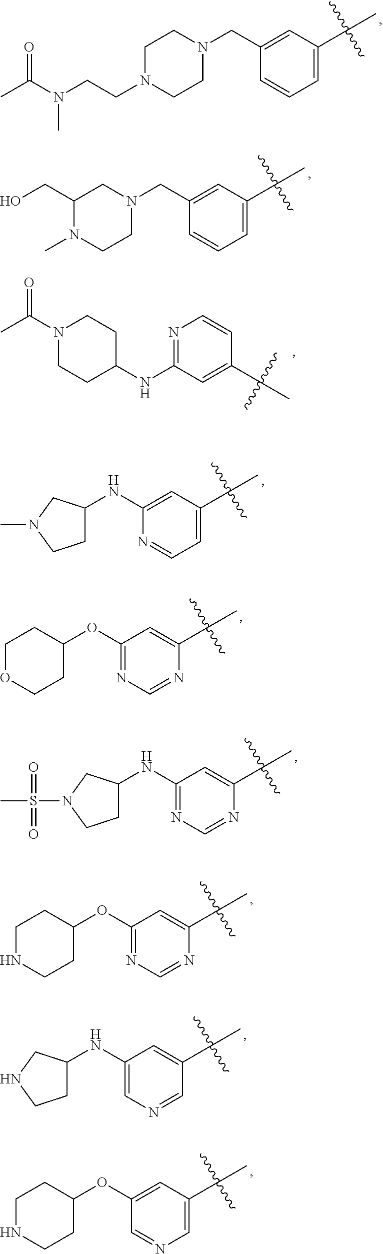

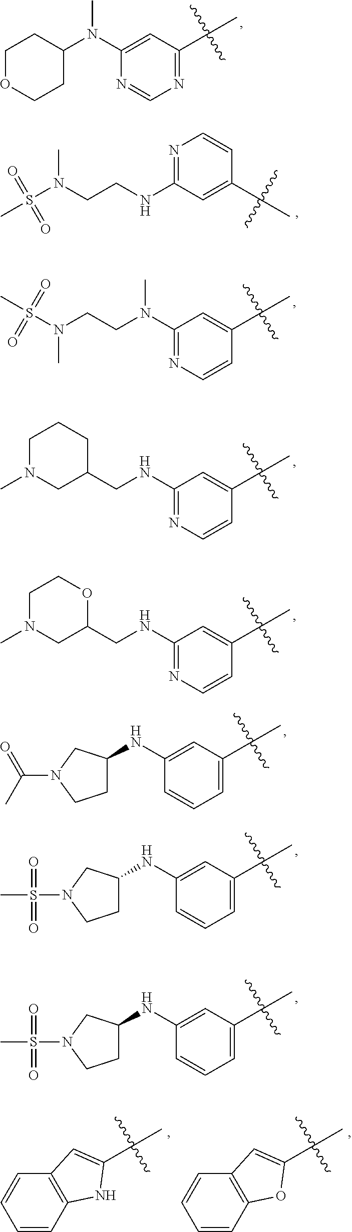

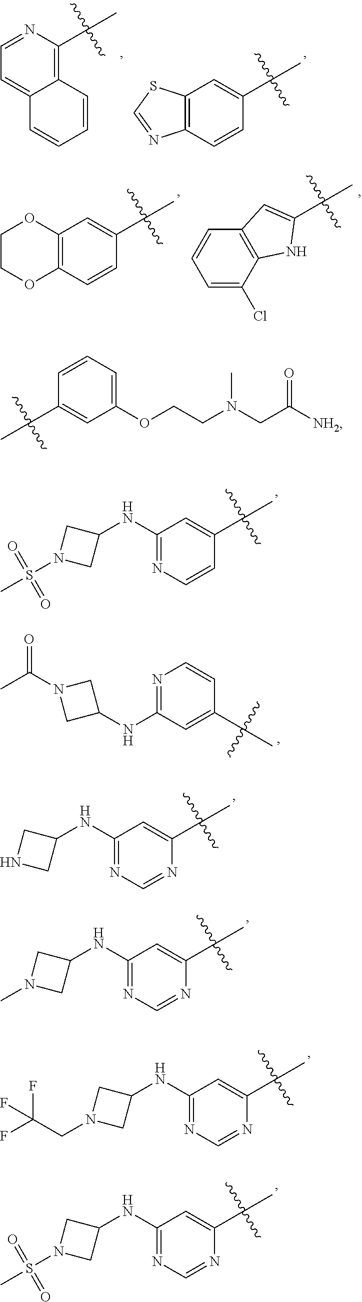

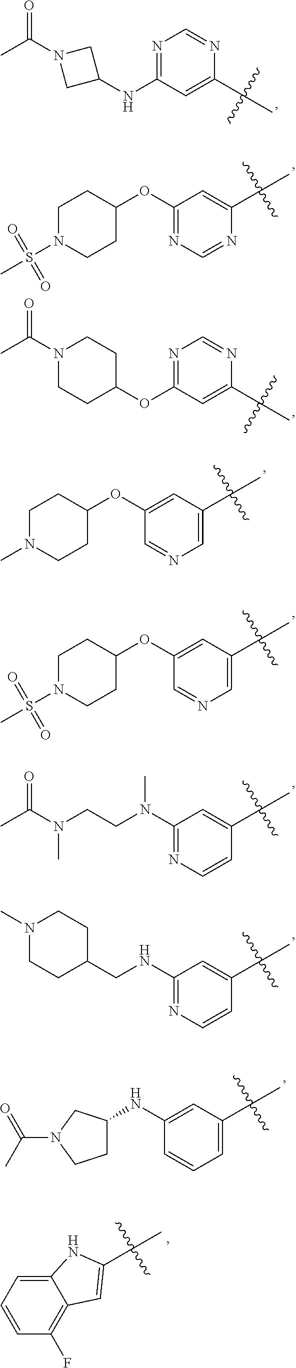

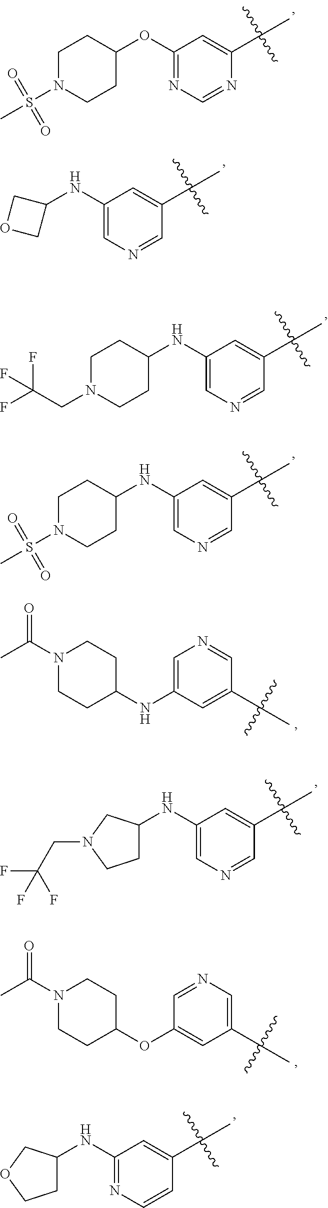

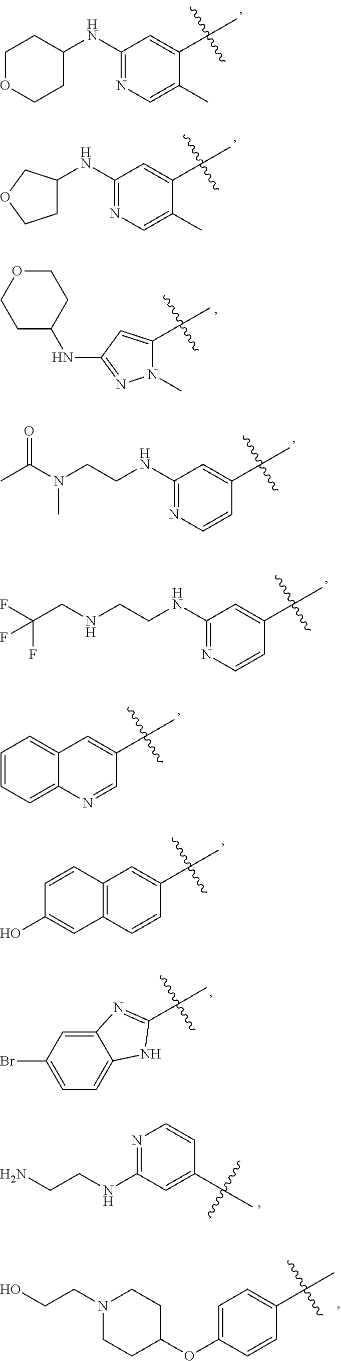

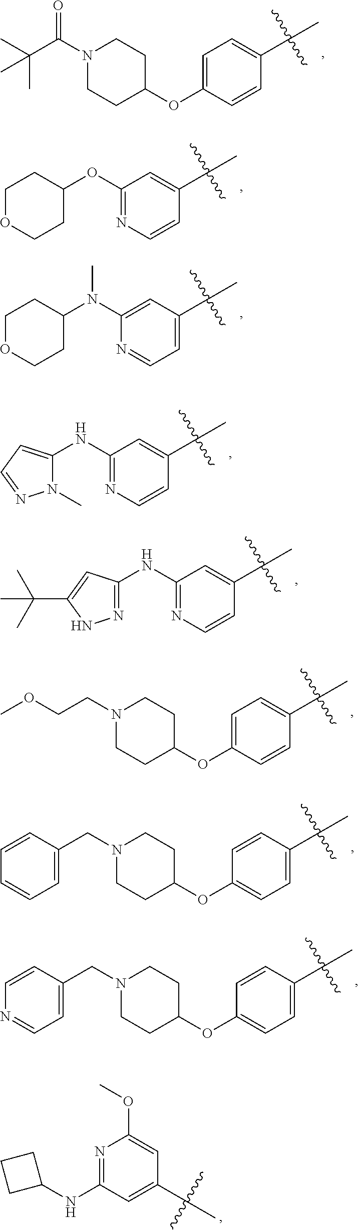

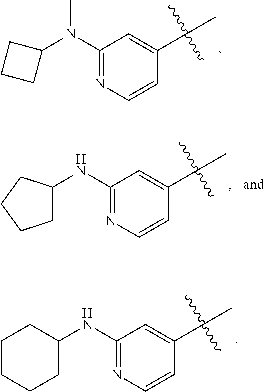 [SMILES: *c1ccnc(N(C)C2CCC2)c1 ; *c1ccnc(NC2CCCC2)c1 ; *c1ccnc(NC2CCCCC2)c1]

In certain embodiments, Ring Z, e.g., Ar, Cy^{A}, Ring A, and the like, is selected from the group consisting of:

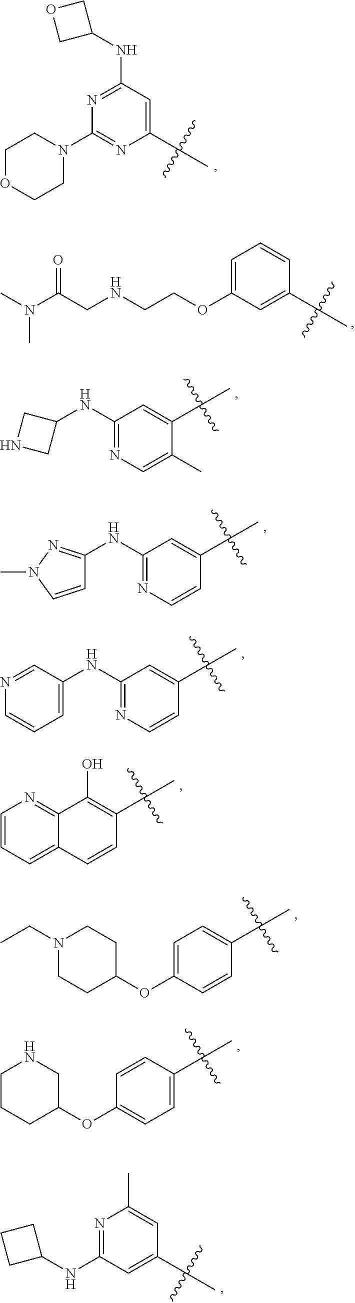

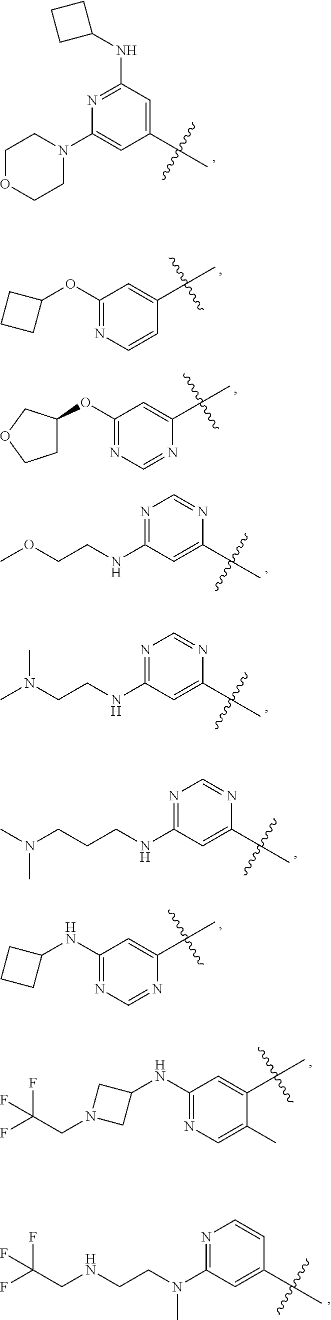

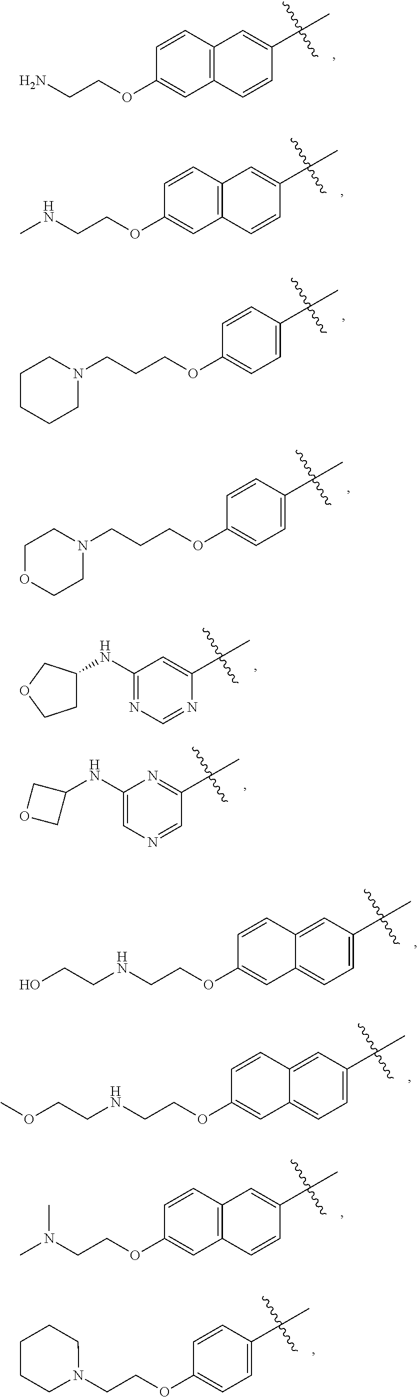

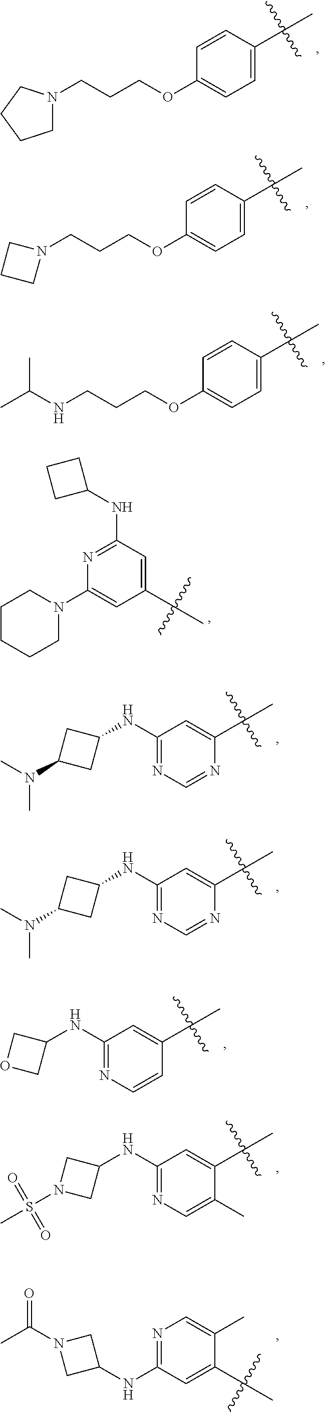

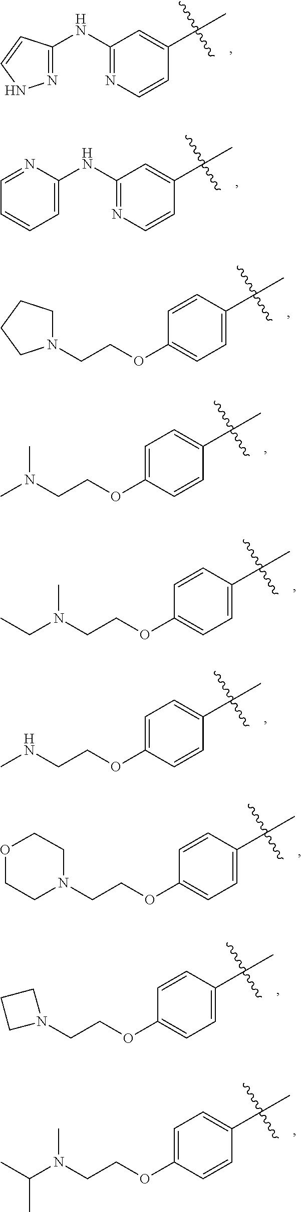

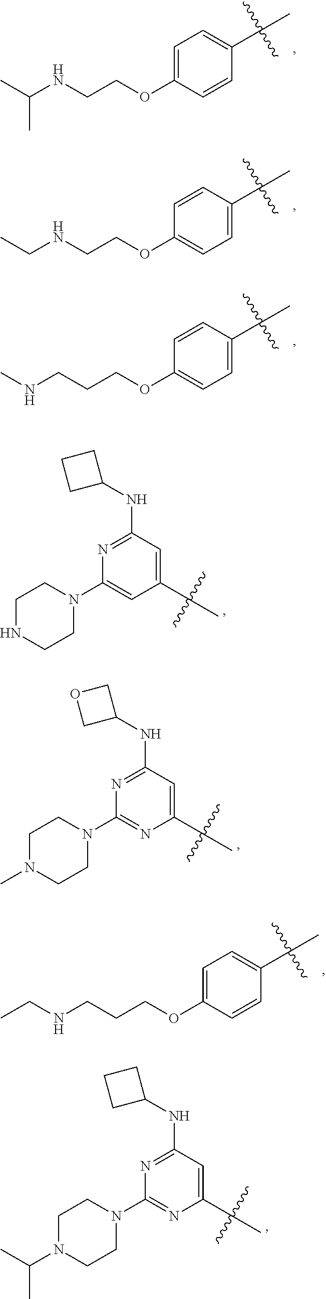

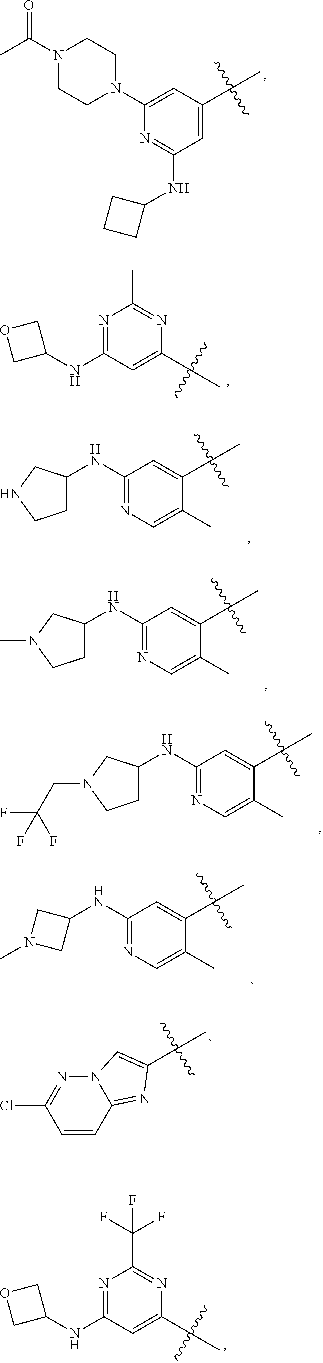

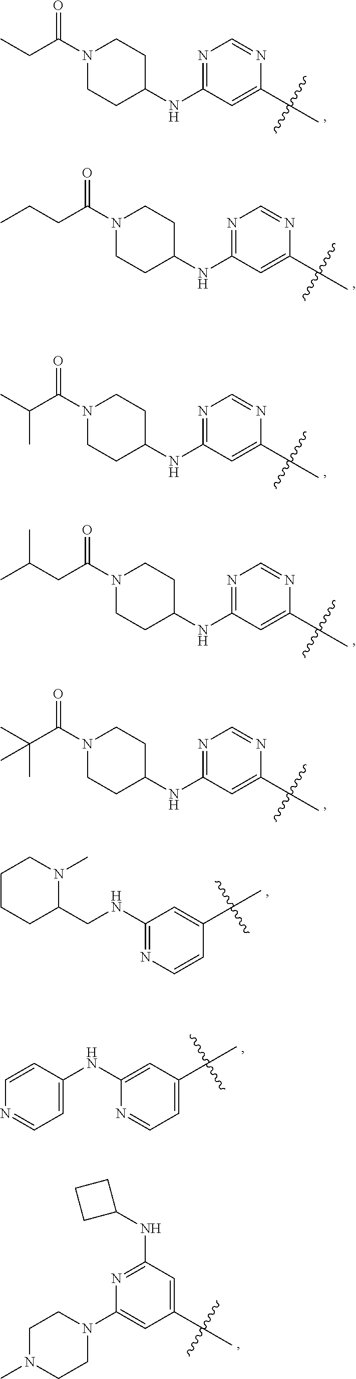

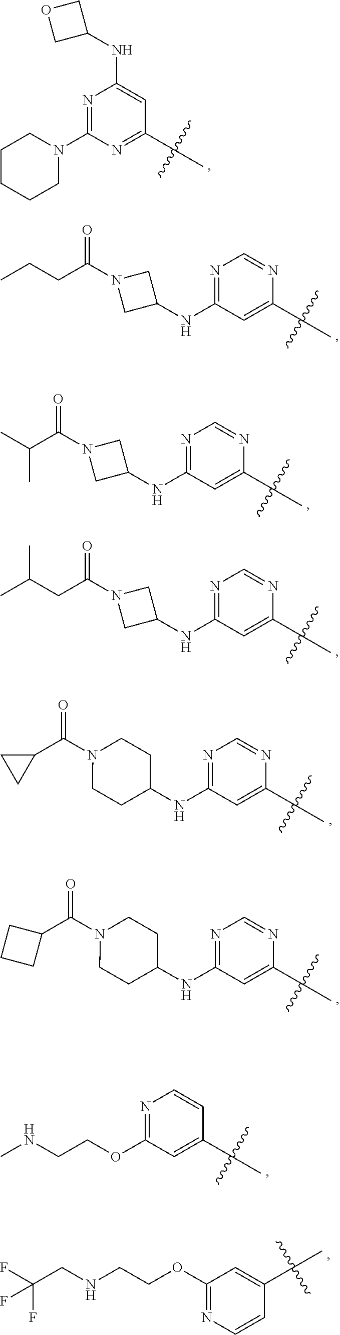

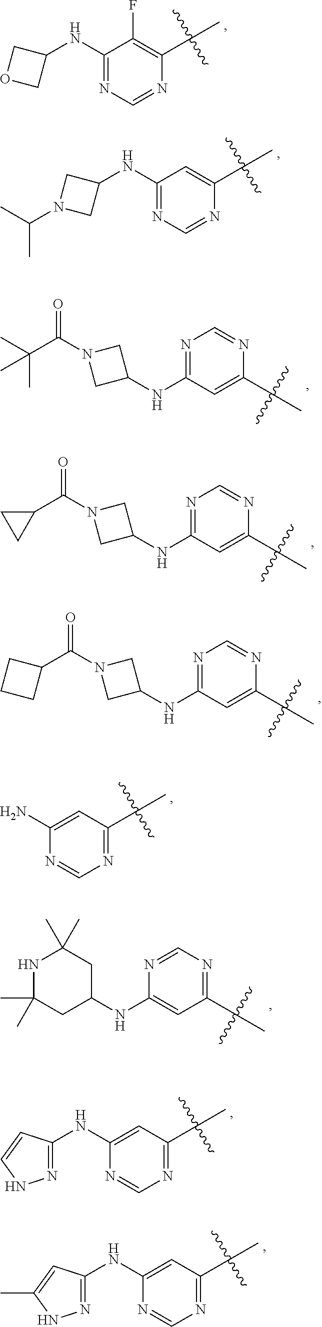

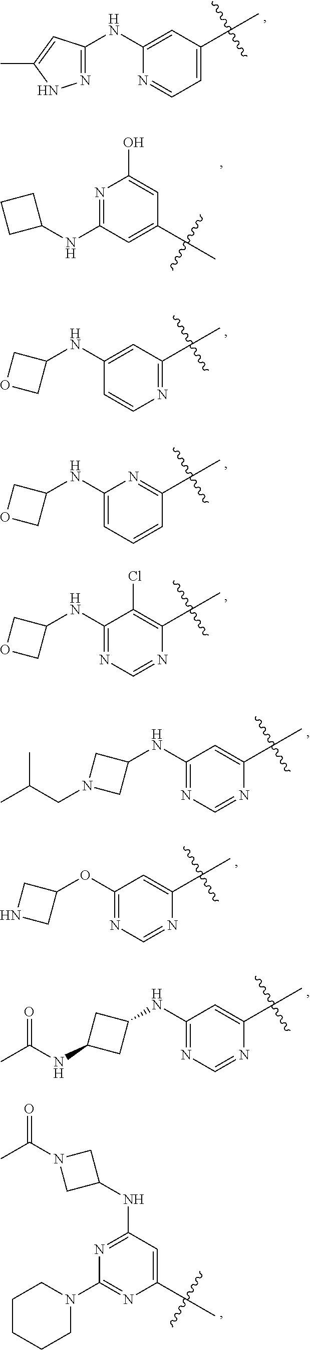

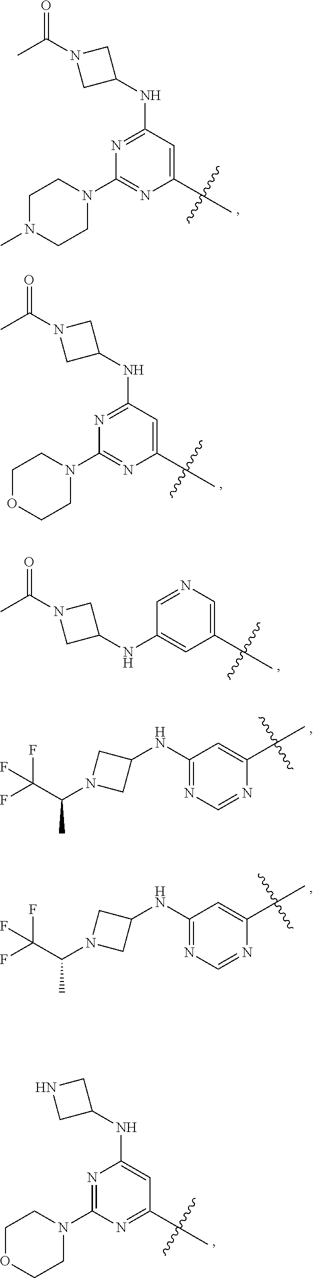

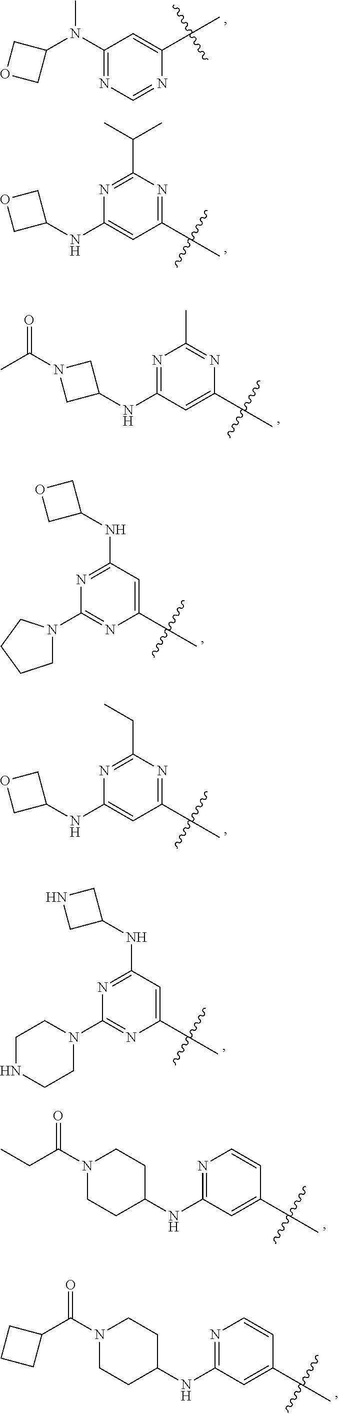

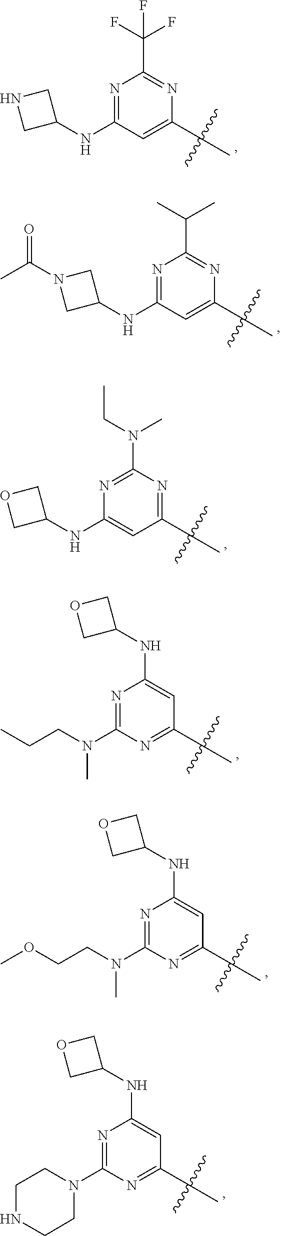

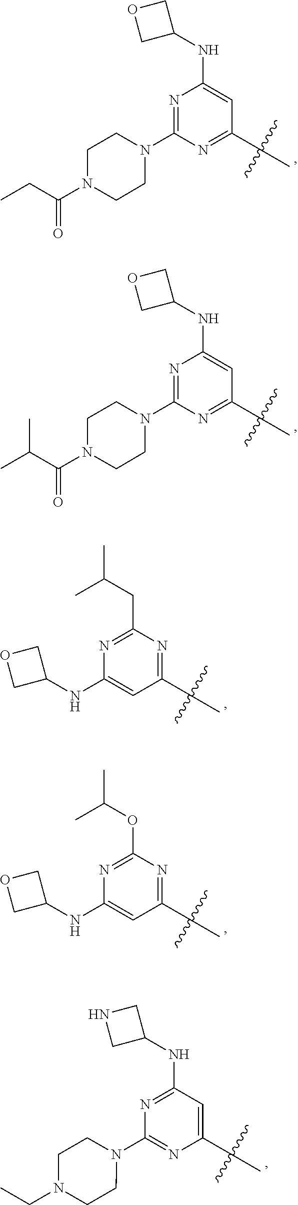 [SMILES: *c1cc(NC2COC2)nc(N2CCN(C(=O)CC)CC2)n1 ; *c1cc(NC2COC2)nc(N2CCN(C(=O)C(C)C)CC2)n1 ; *c1cc(NC2COC2)nc(CC(C)C)n1 ; *c1cc(NC2COC2)nc(OC(C)C)n1 ; *c1cc(NC2CNC2)nc(N2CCN(CC)CC2)n1]

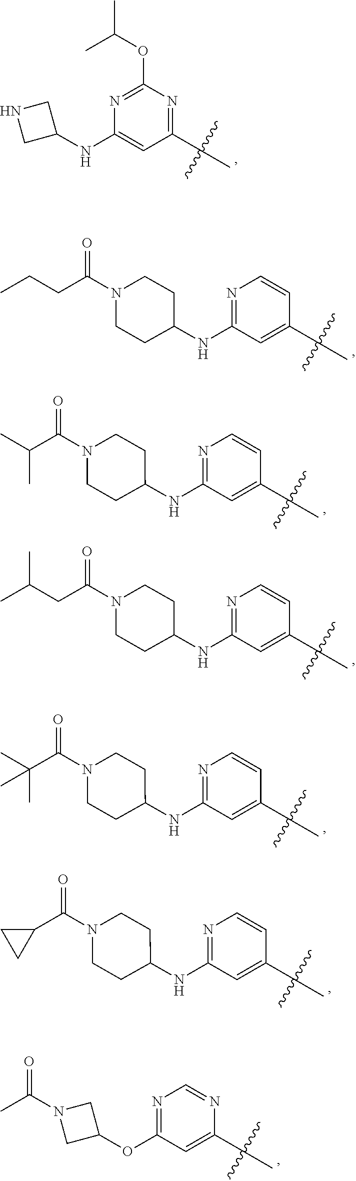

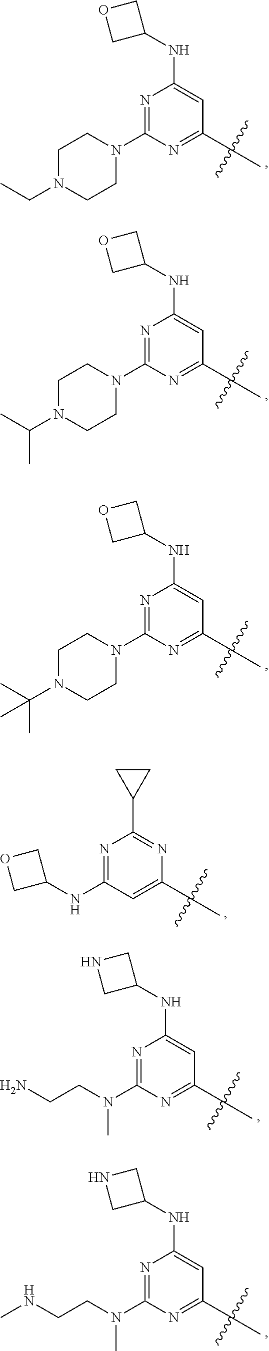

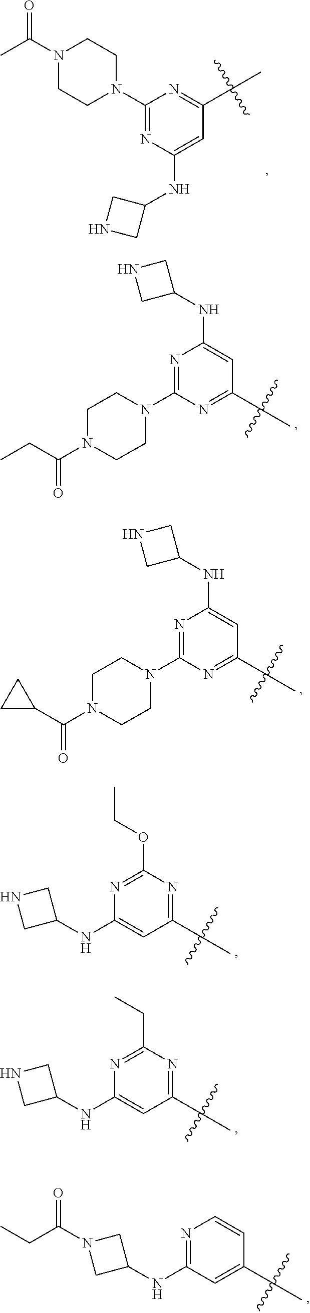

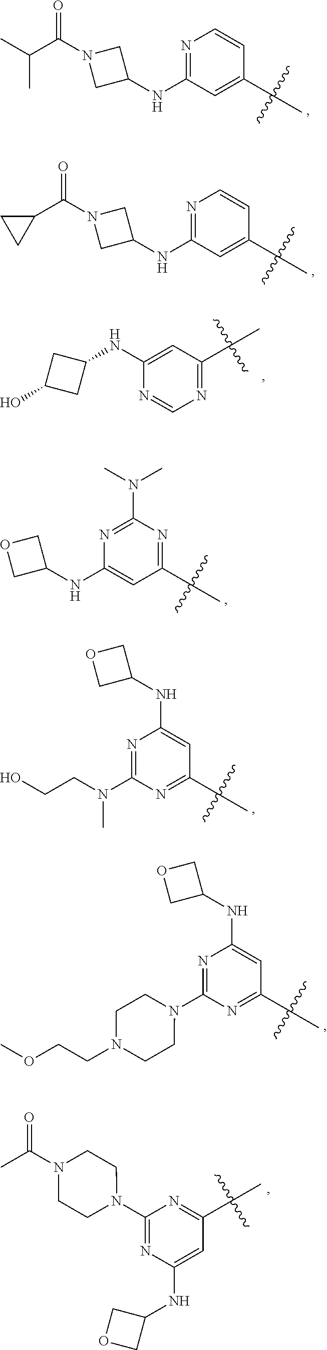

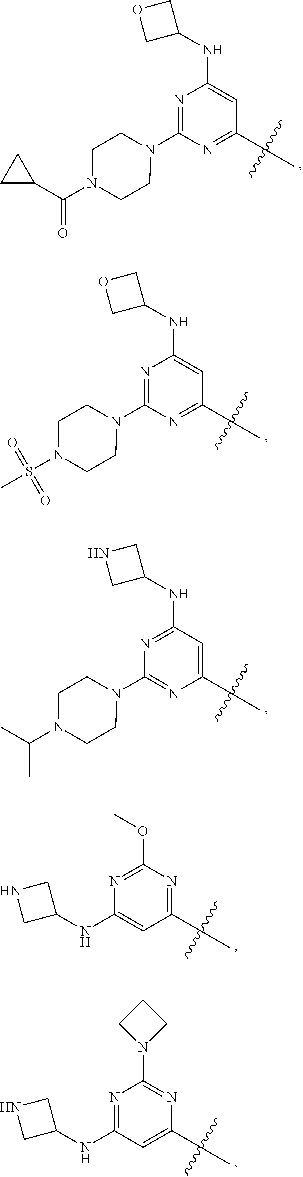 [SMILES: *c1cc(NC2COC2)nc(N2CCN(C(=O)C3CC3)CC2)n1 ; *c1cc(NC2COC2)nc(N2CCN(S(C)(=O)=O)CC2)n1 ; *c1cc(NC2CNC2)nc(N2CCN(C(C)C)CC2)n1 ; *c1cc(NC2CNC2)nc(OC)n1 ; *c1cc(NC2CNC2)nc(N2CCC2)n1]

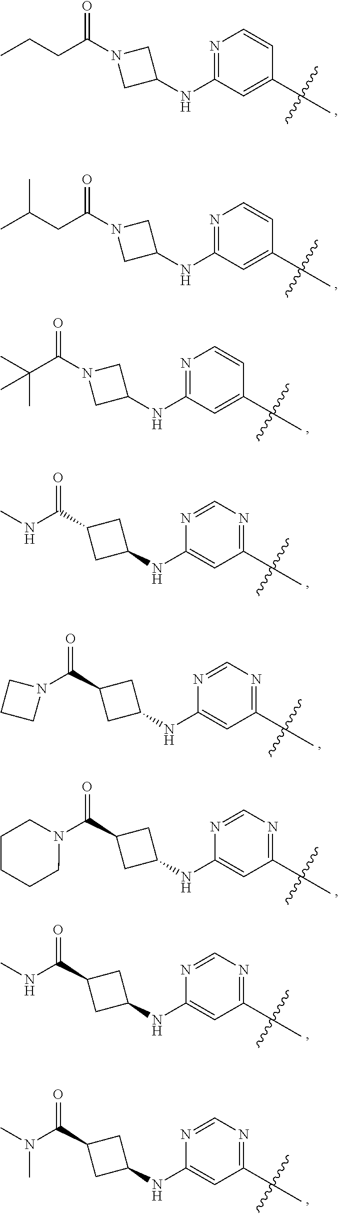

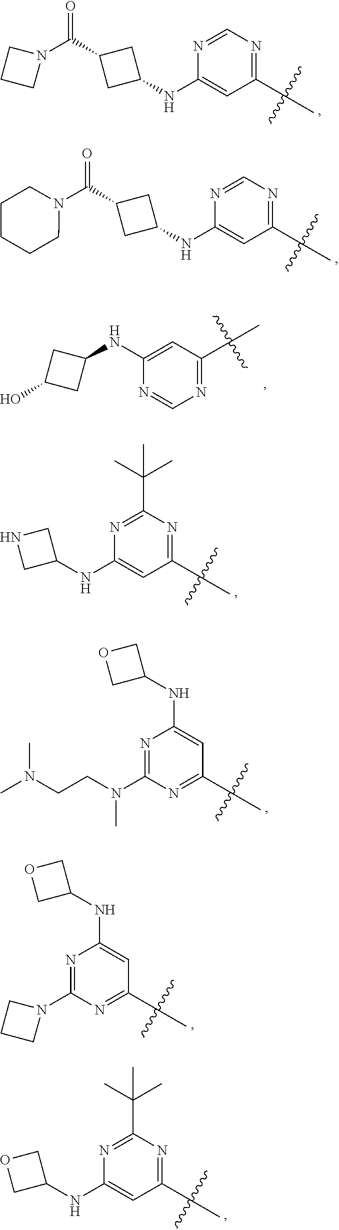

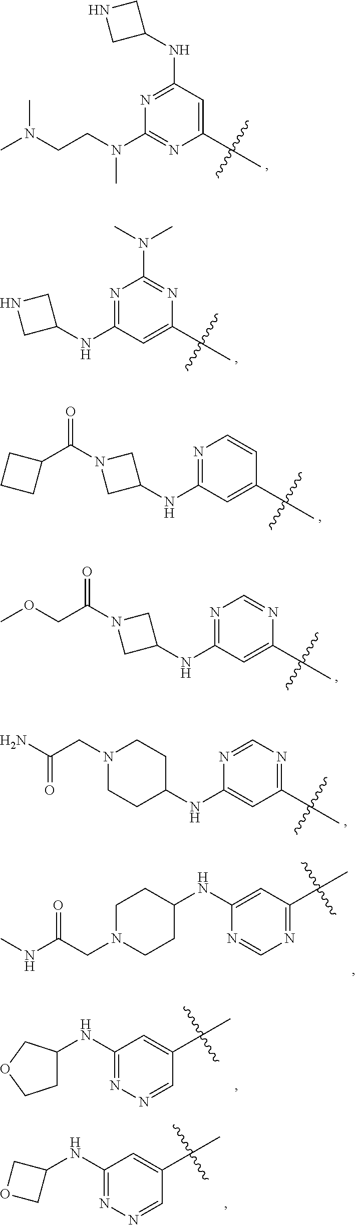

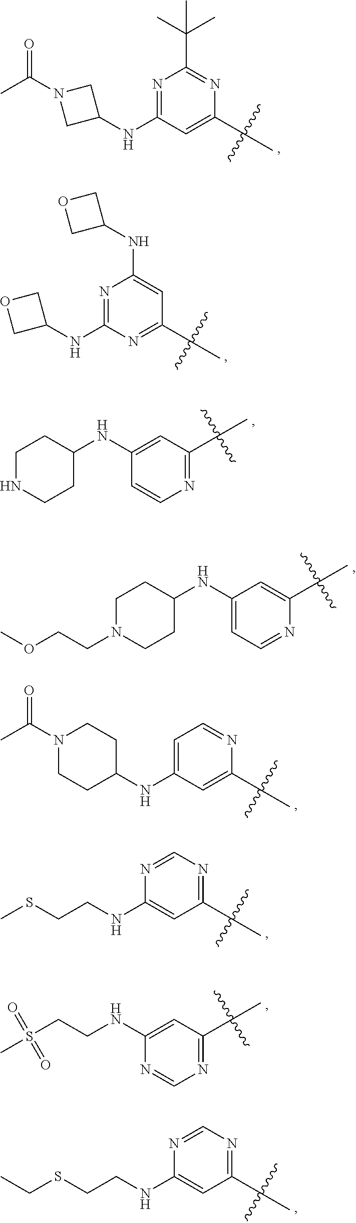

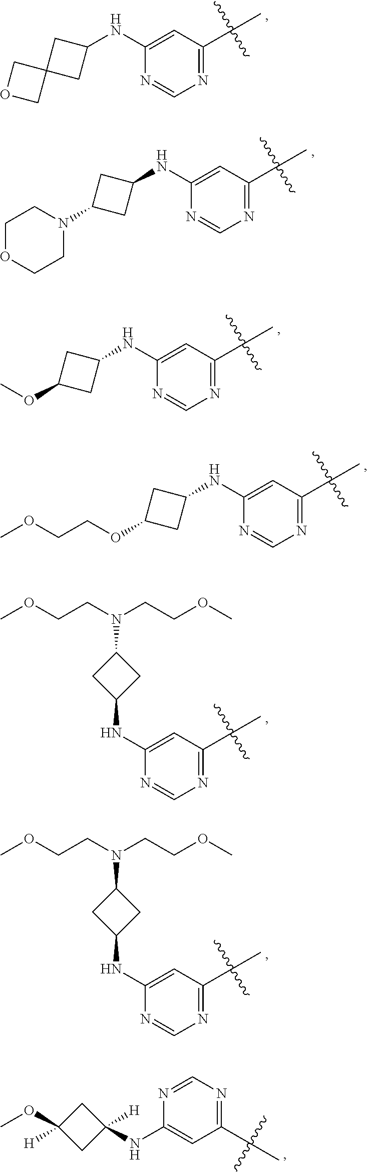

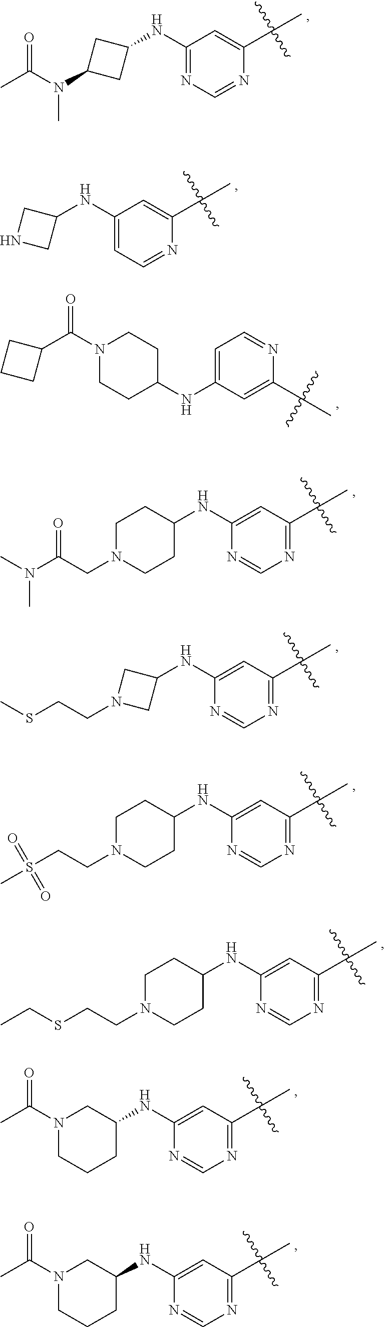

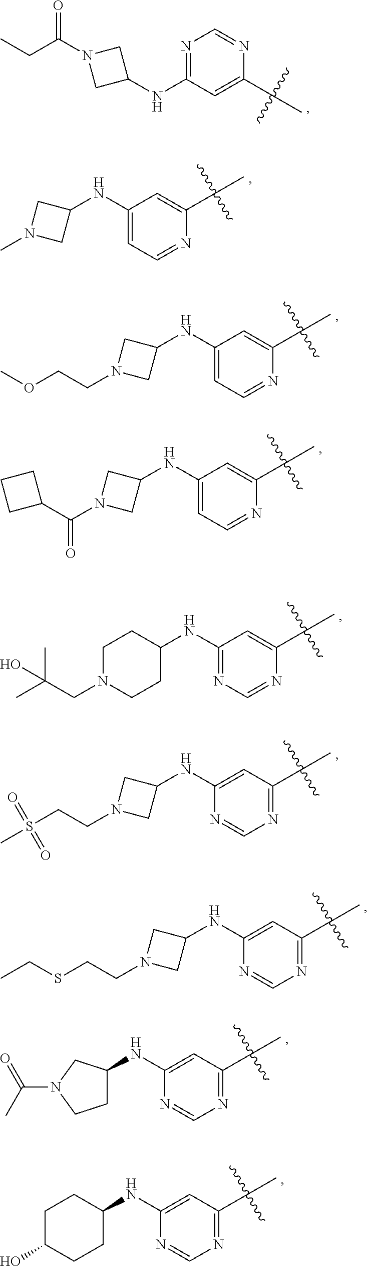

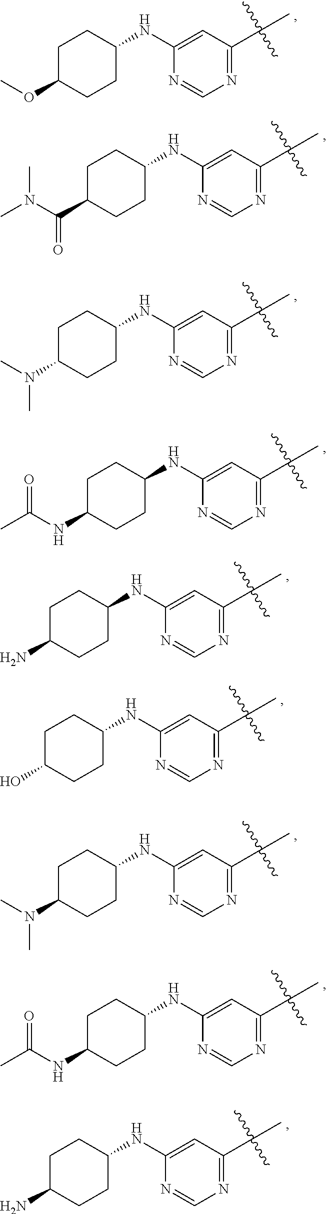

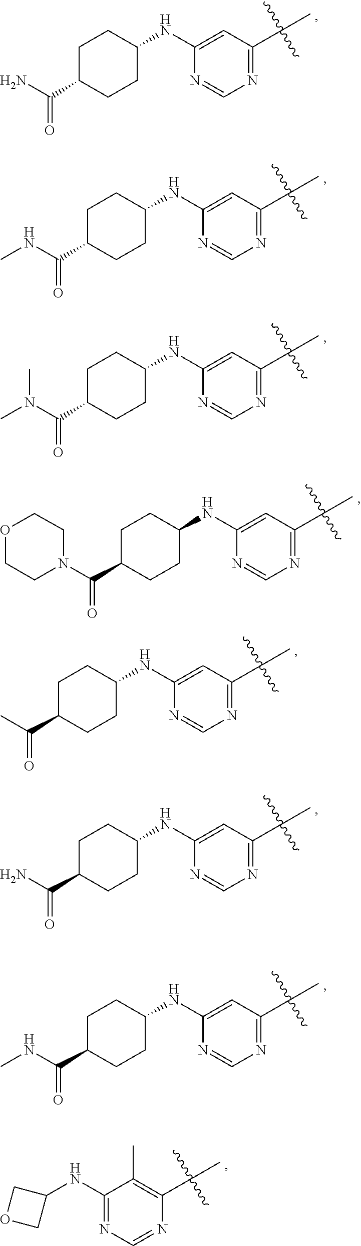

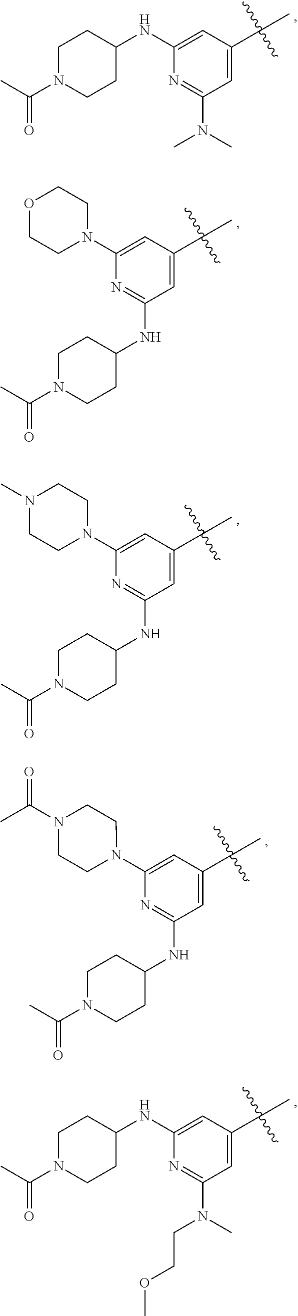

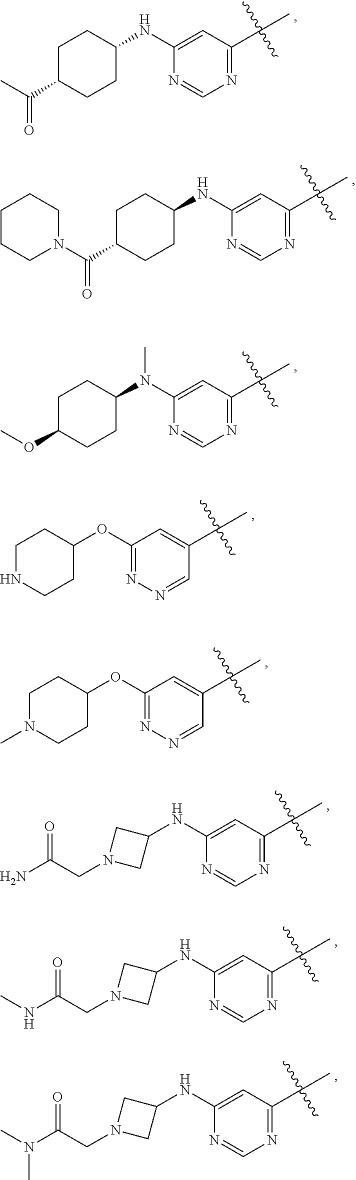

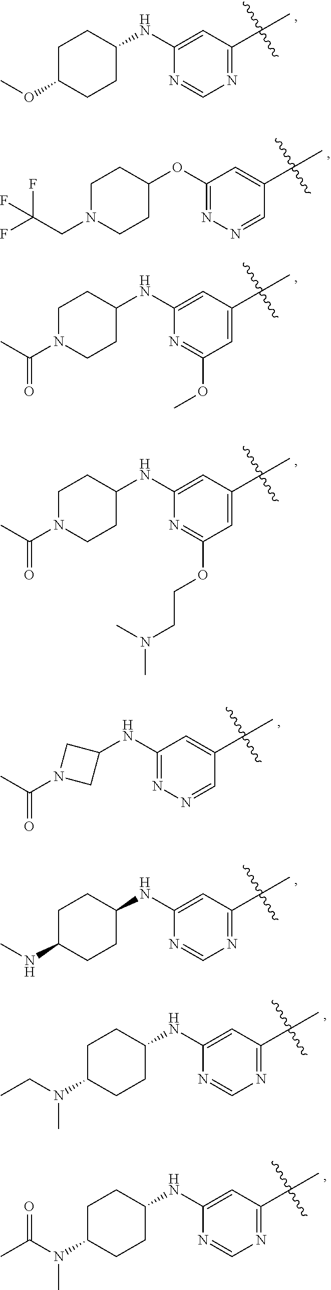

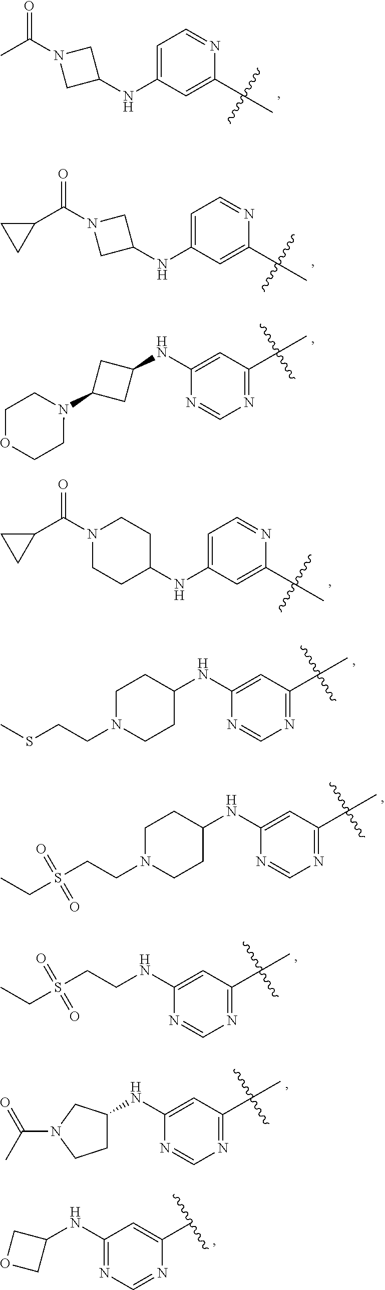

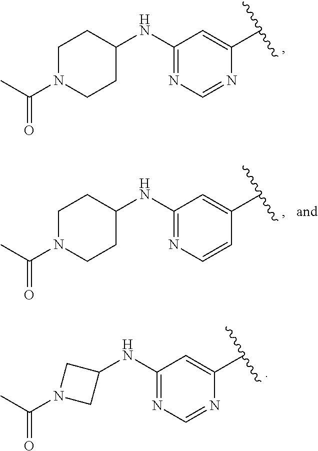 [SMILES: *c1cc(NC2CCN(C(C)=O)CC2)ncn1 ; *c1ccnc(NC2CCN(C(C)=O)CC2)c1 ; *c1cc(NC2CN(C(C)=O)C2)ncn1]

In certain embodiments, Ring Z, e.g., Ar, Cy^{A}, Ring A, and the like, is selected from the group consisting of:

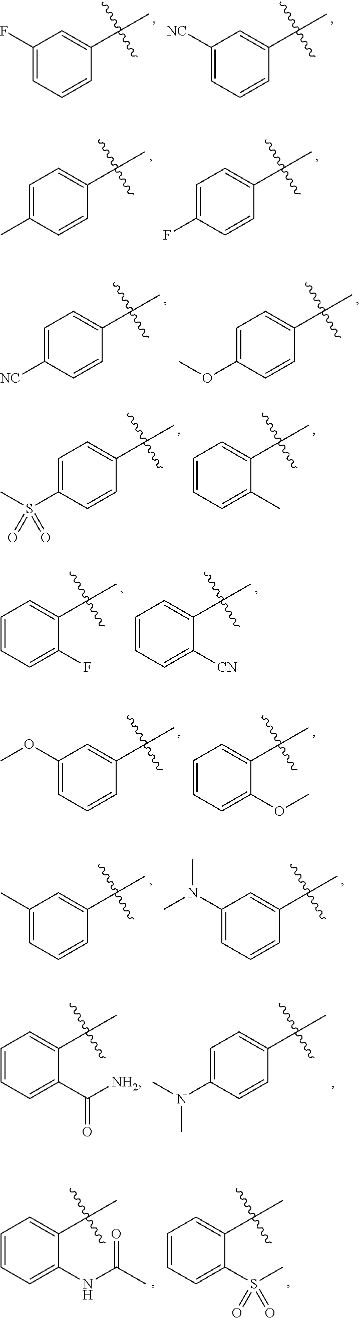

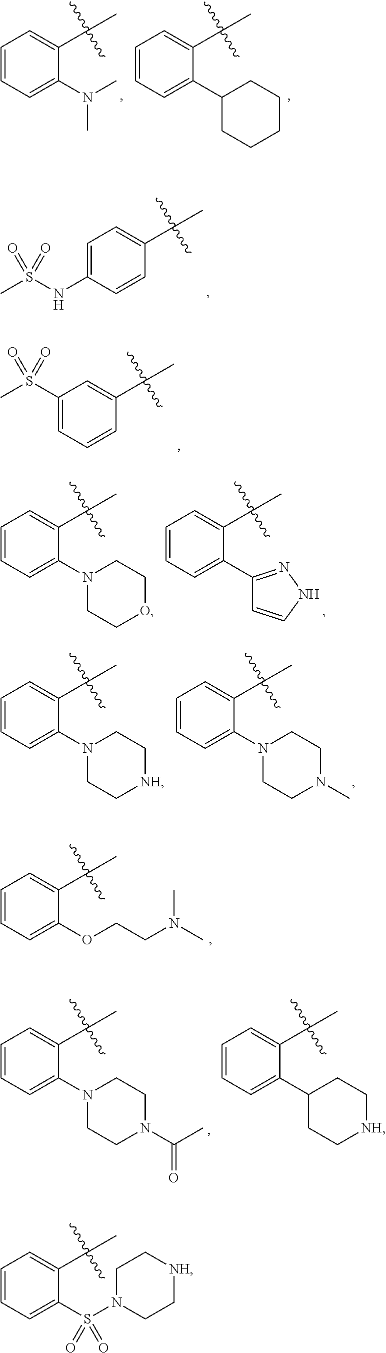

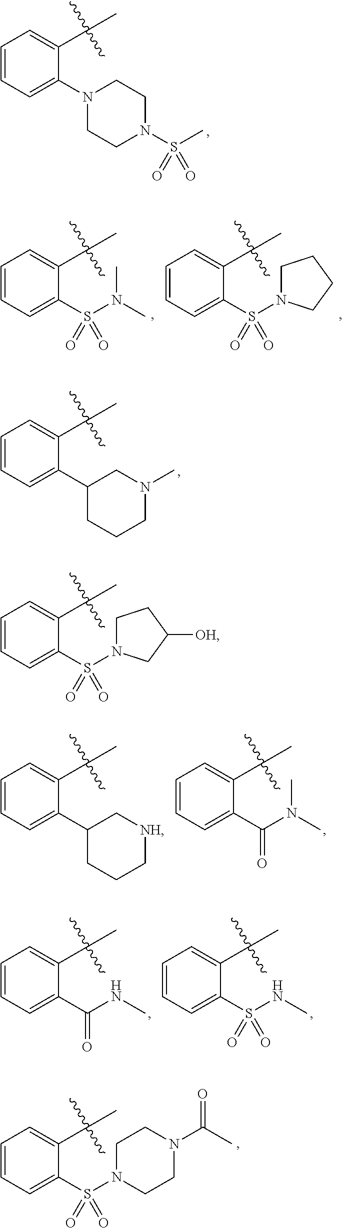

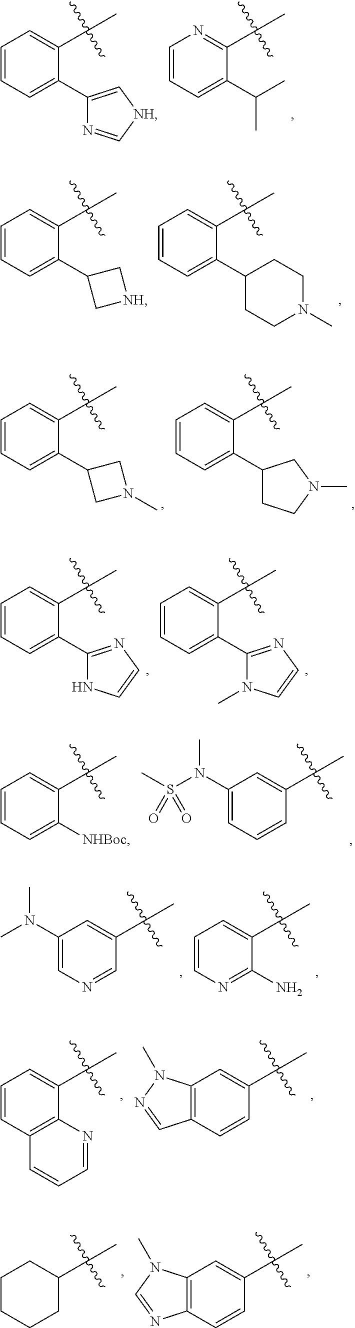

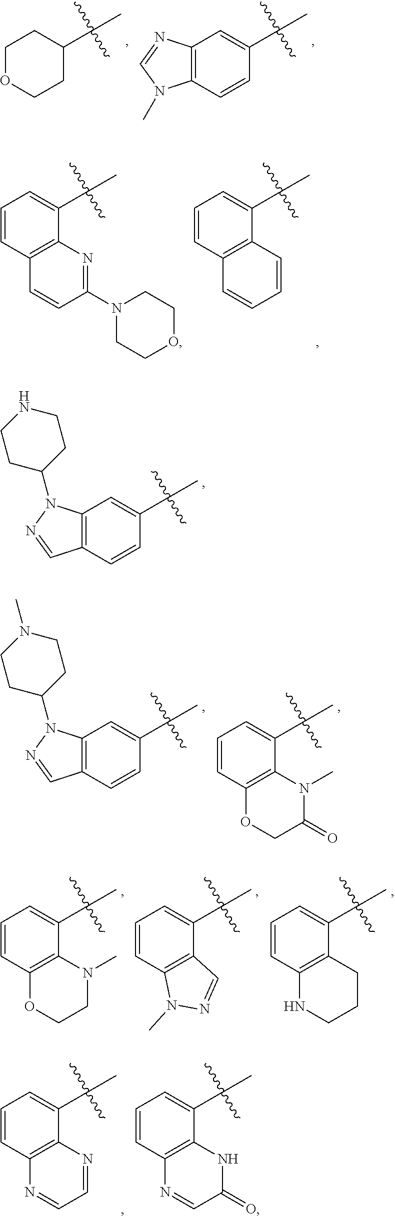

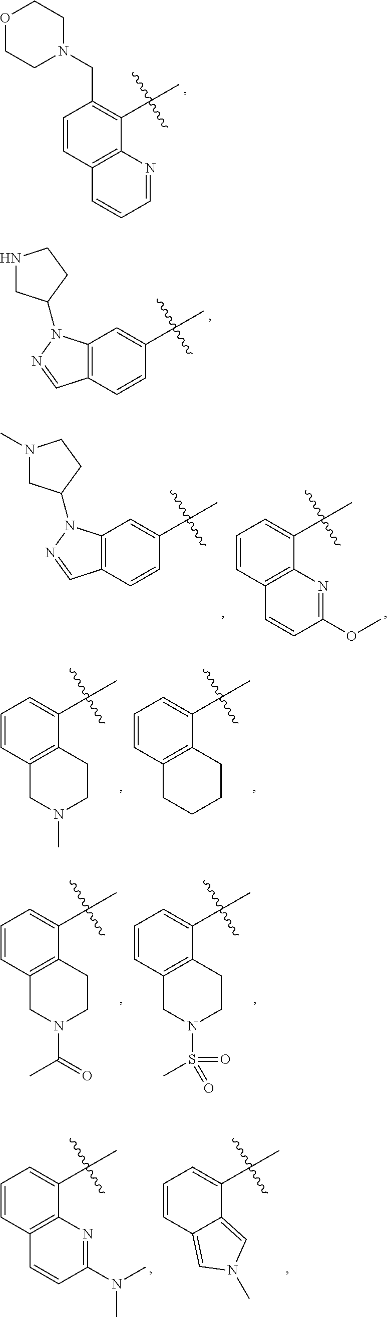

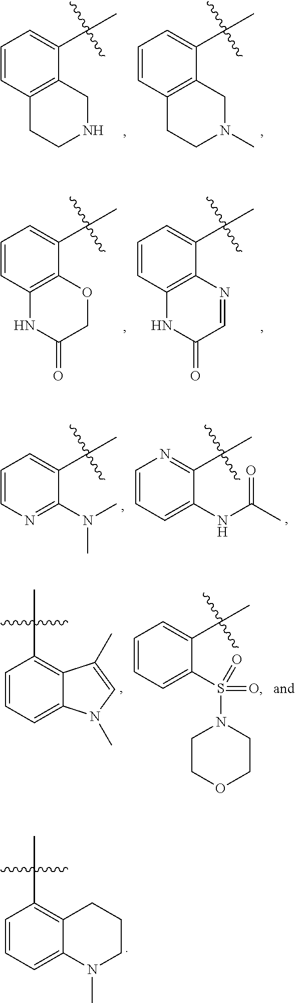

In certain embodiments, Ring Z, e.g., Ar, Cy^{A}, Ring A, and the like, is selected from the group consisting of:

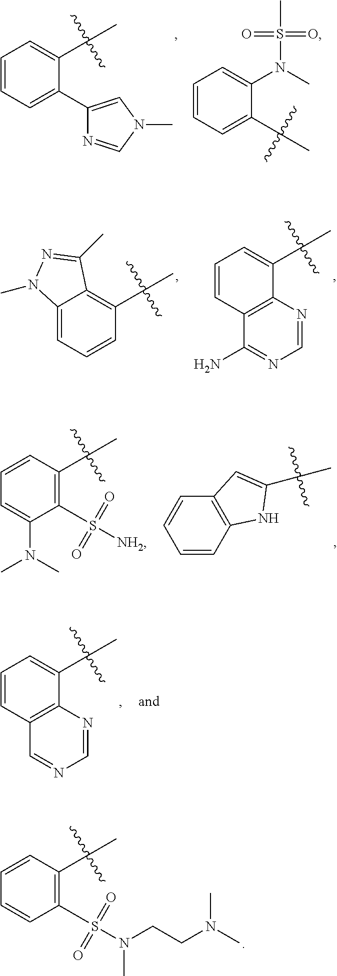

In certain embodiments, Ring Z, e.g., Ar, Cy^{A}, Ring A, and the like, is selected from the group consisting of:

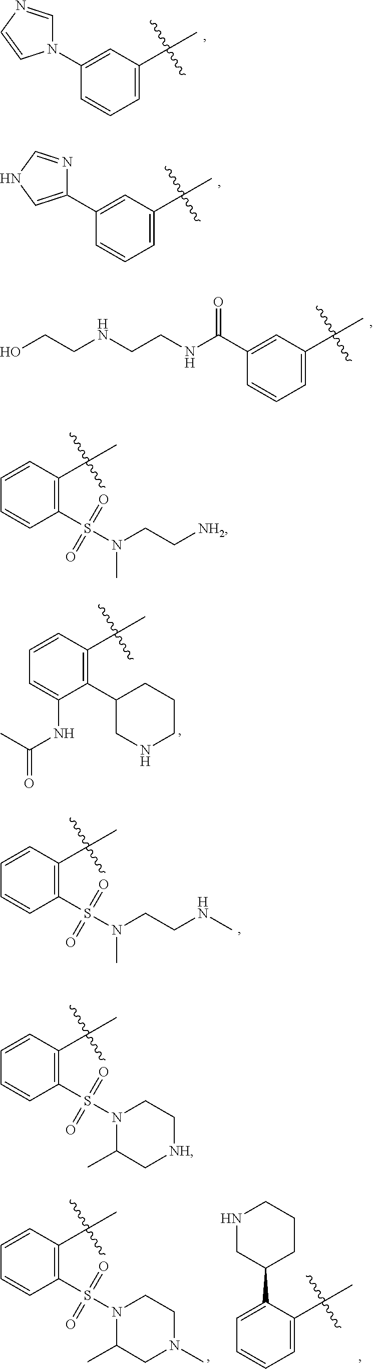

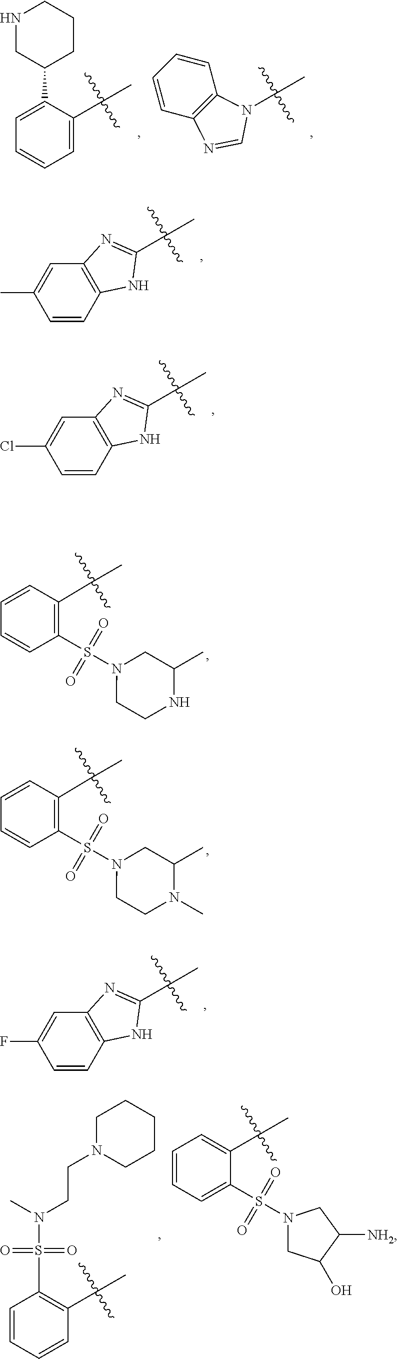

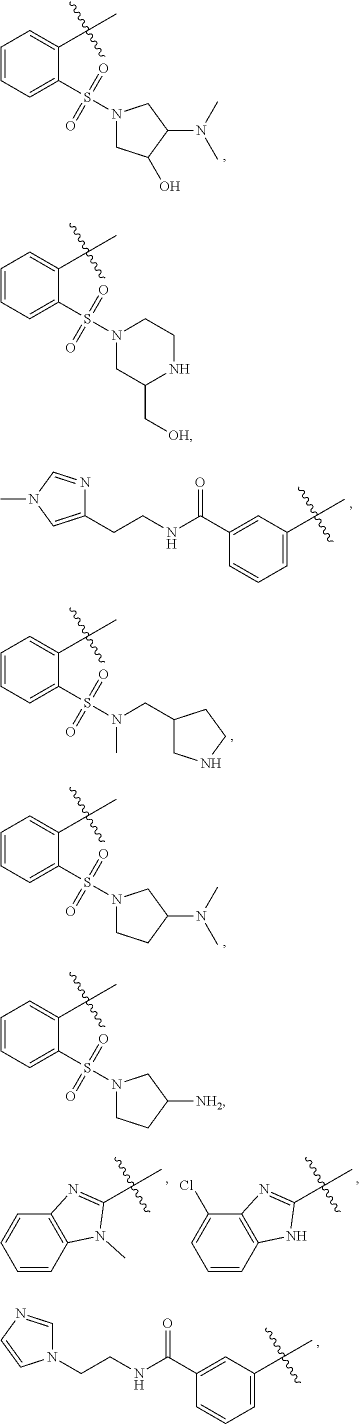

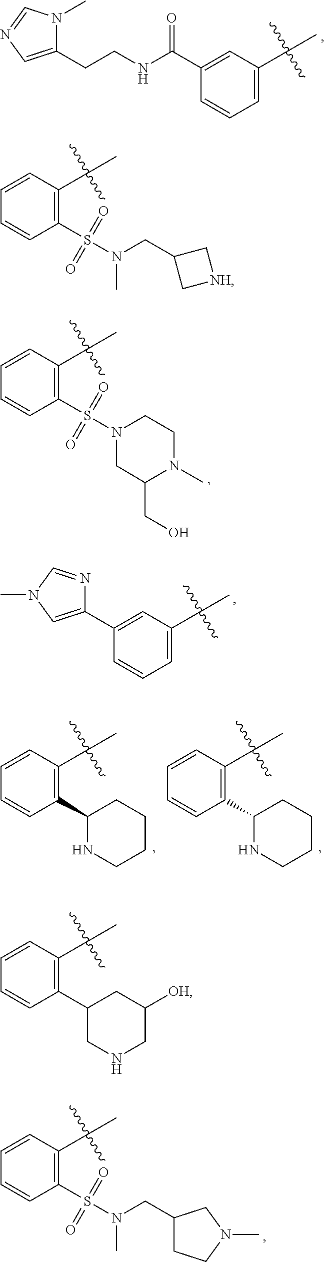

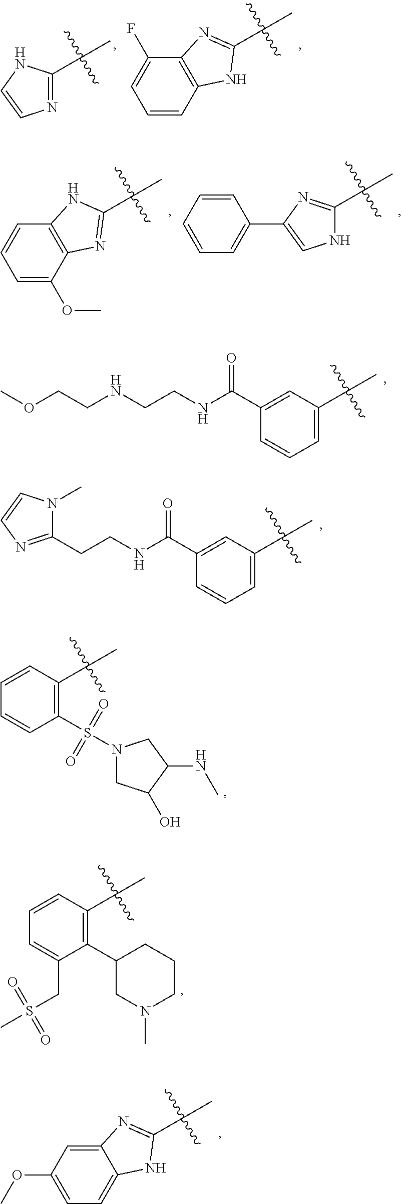

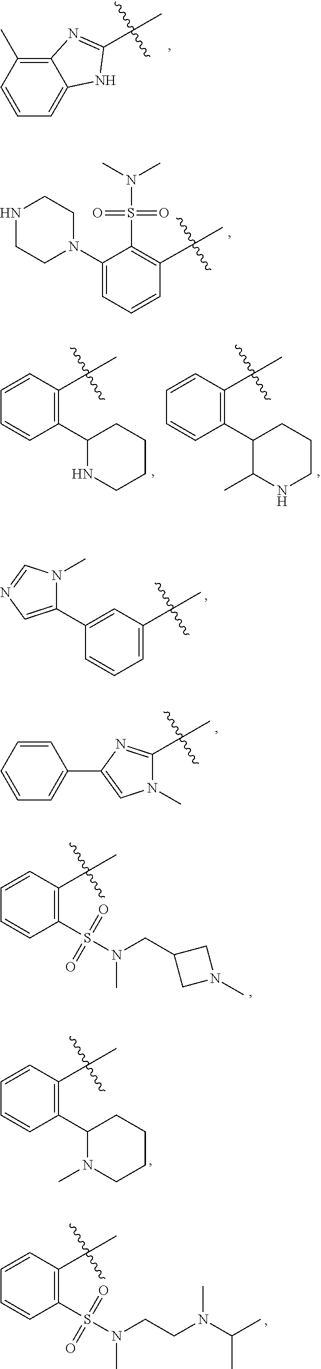

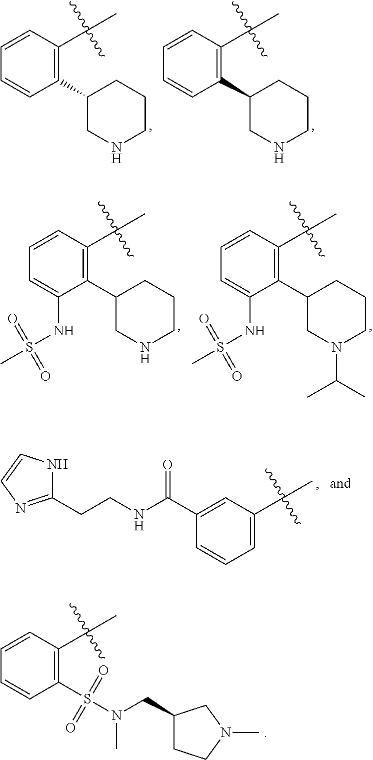

In certain embodiments, Ring Z, e.g., Ar, Cy^{A}, Ring A, and the like, is selected from the group consisting of:

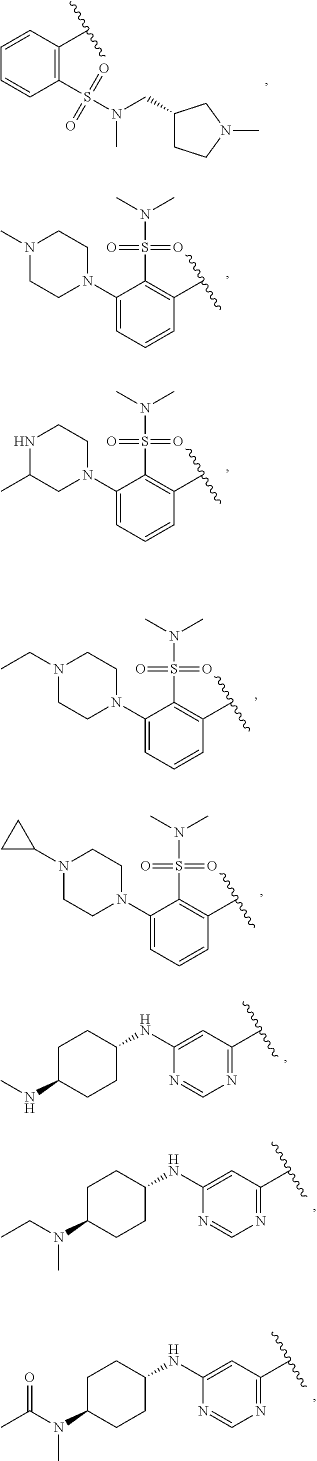

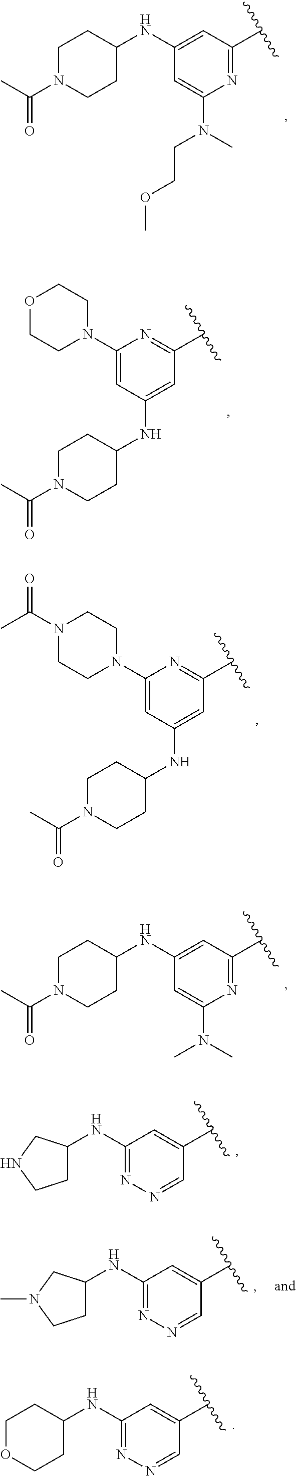

In certain embodiments, Ring Z, e.g., Ar, Cy^{A}, Ring A, and the like, is selected from the group consisting of:

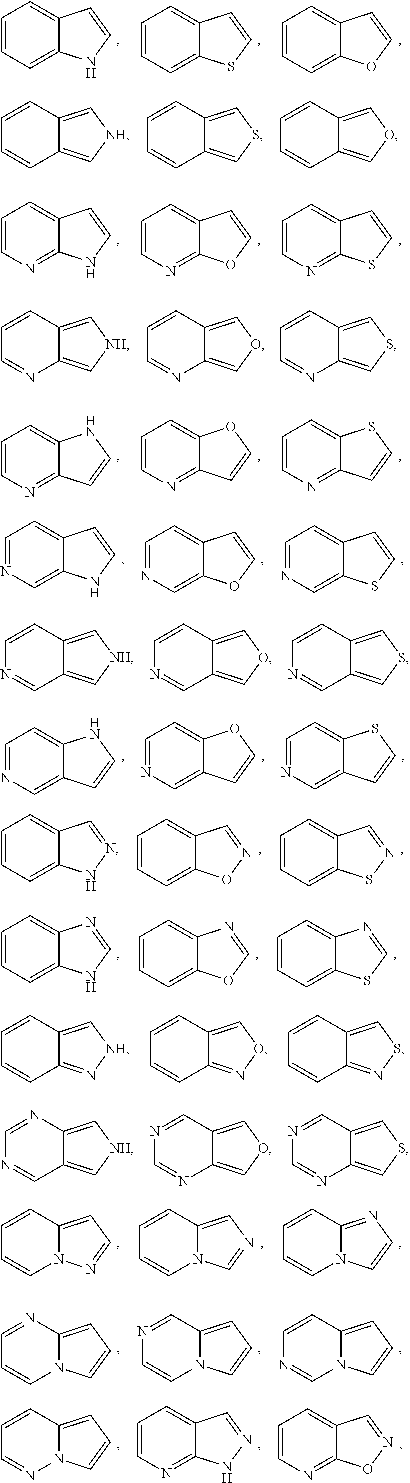

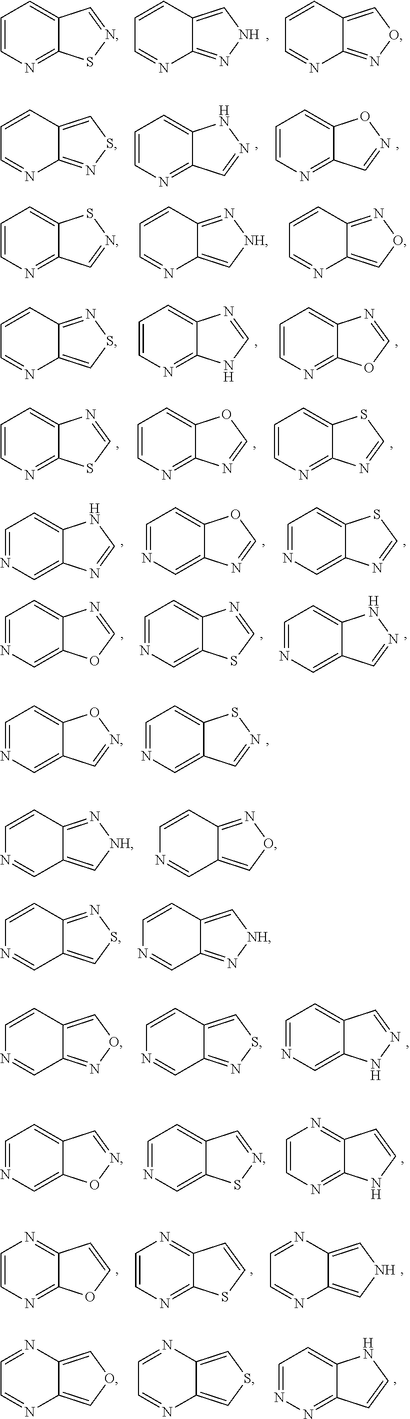

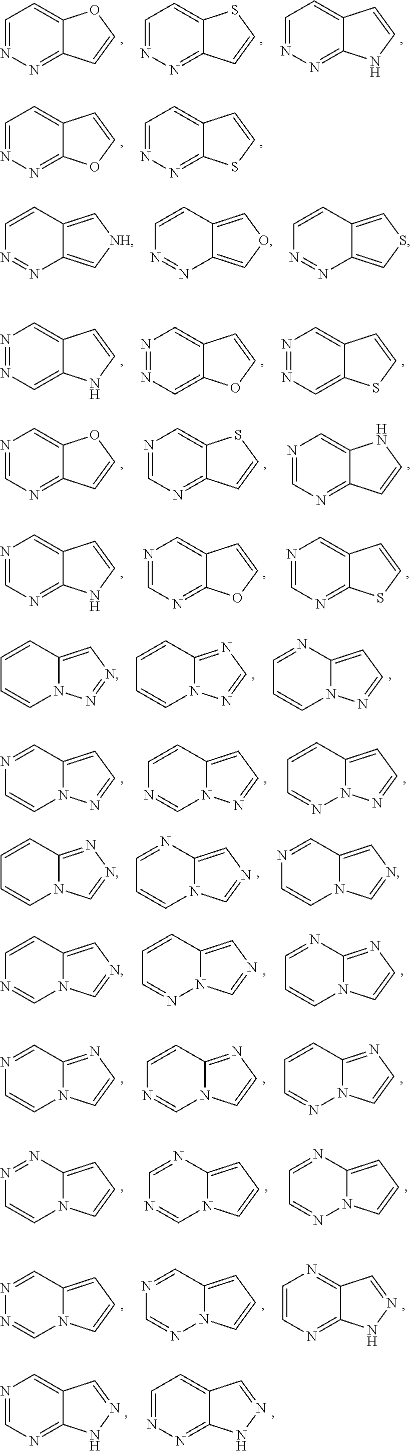

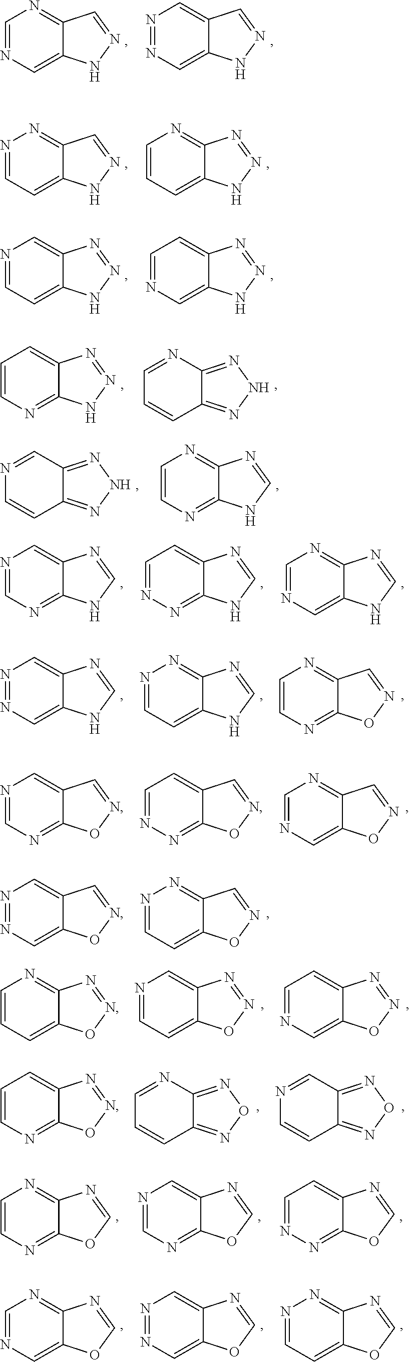

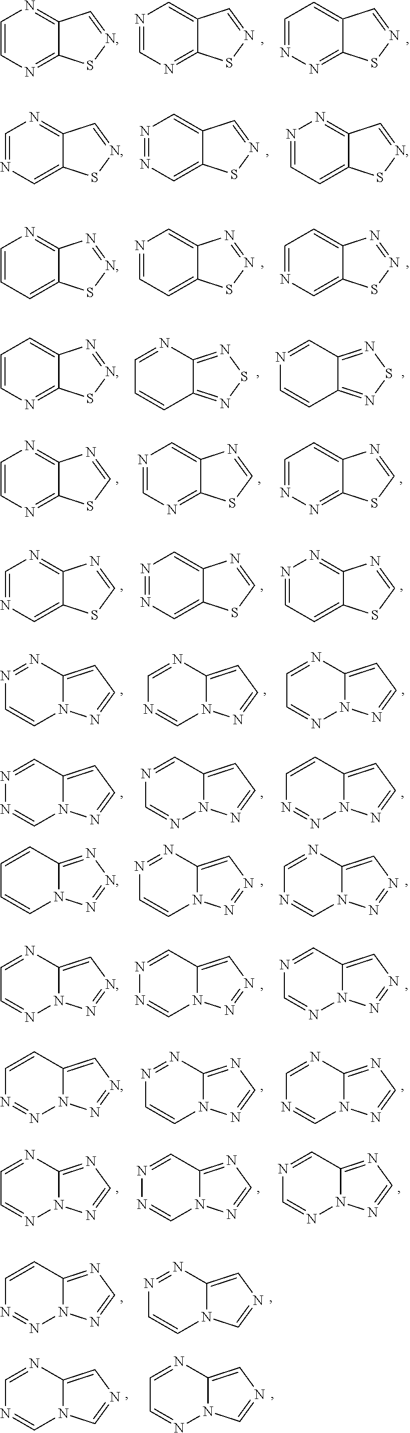

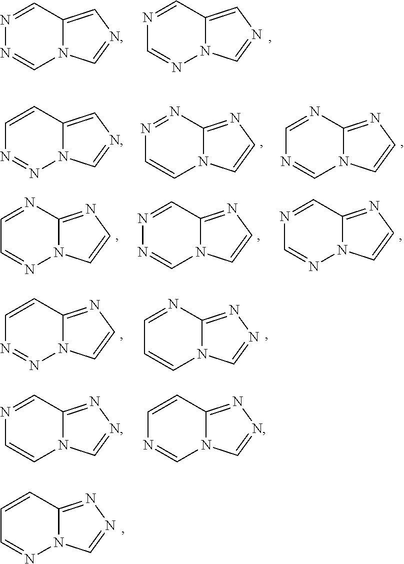

wherein the point of attachment can be any carbon or nitrogen atom, as valency permits, and the ring may be substituted with 0, 1, 2, 3, 4, or 5 R^{y} groups, as valency permits.

In certain embodiments, Ring Z, e.g., Ar, Cy^{A}, Ring A, and the like, is selected from the group consisting of:

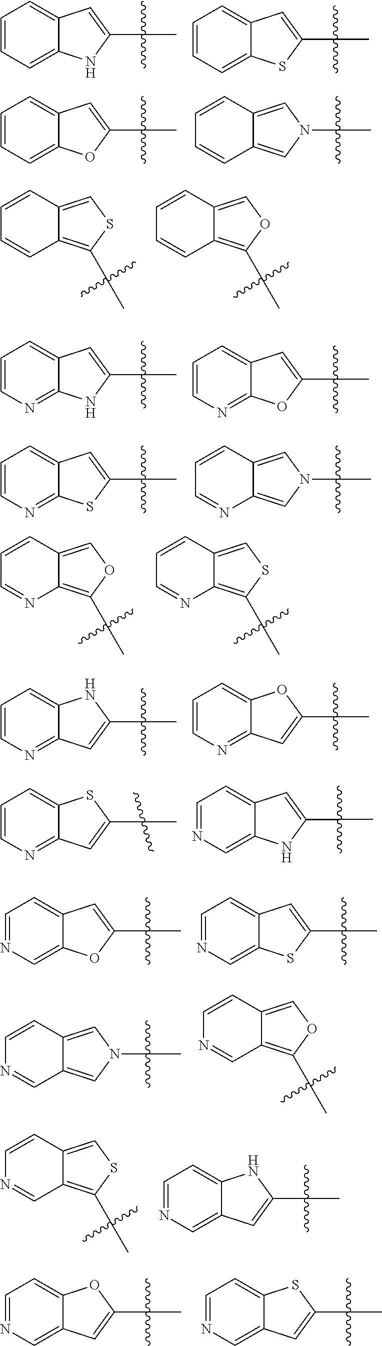

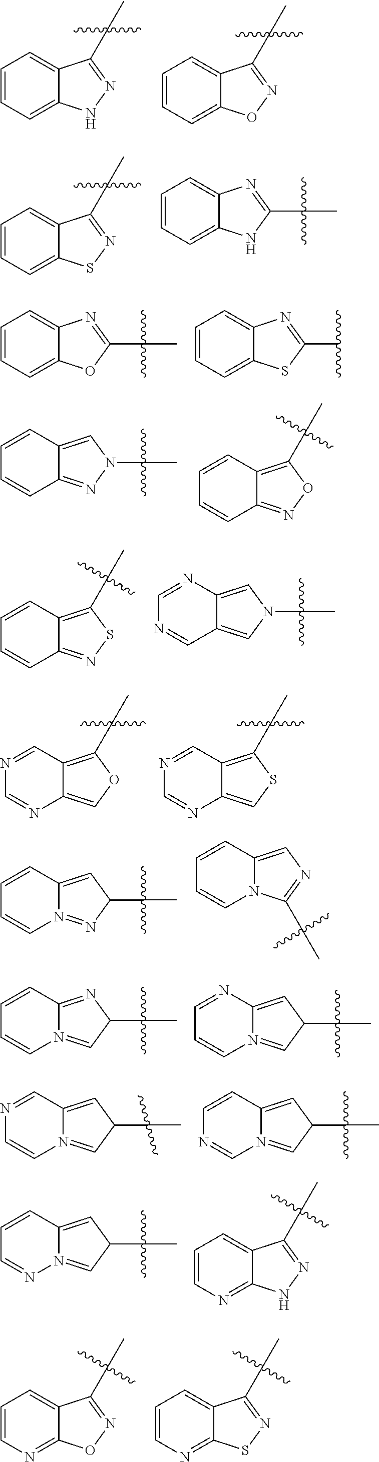

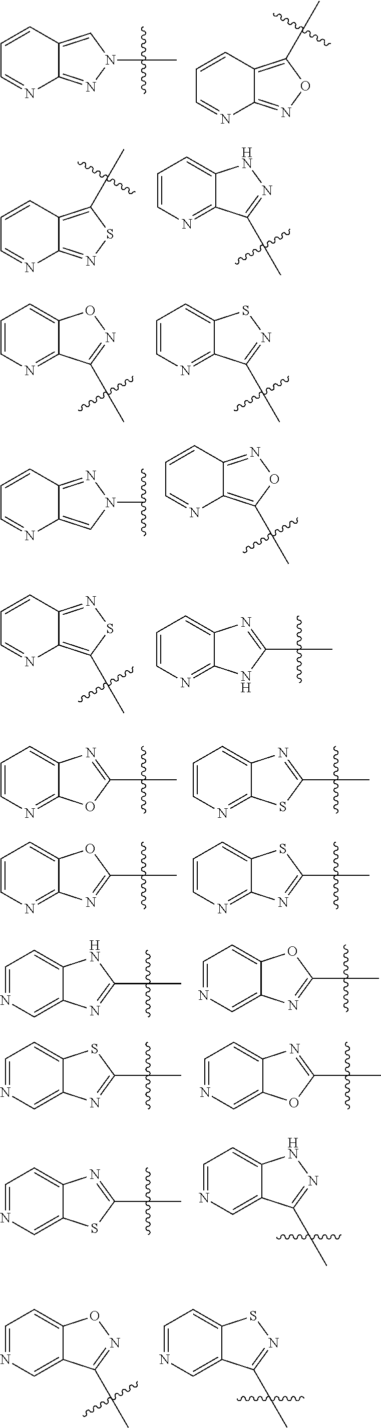

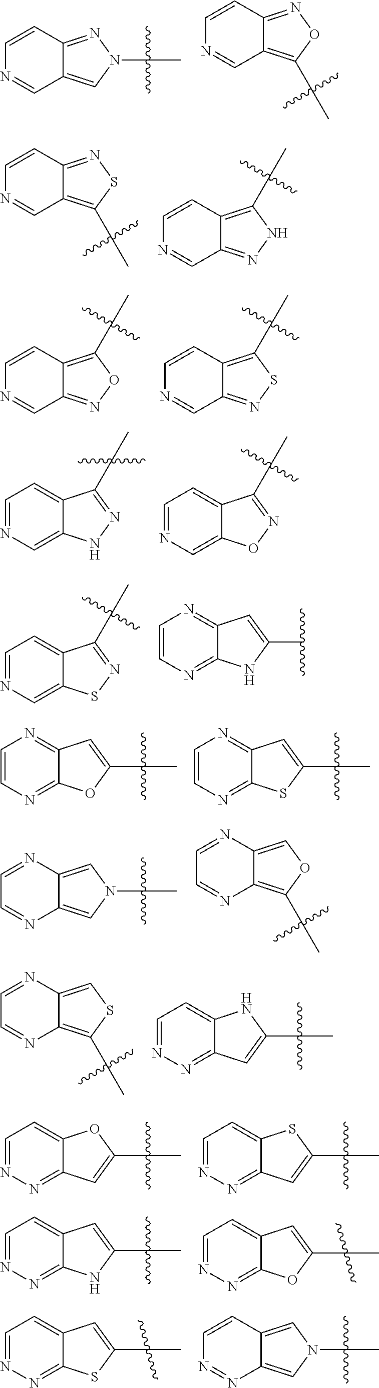

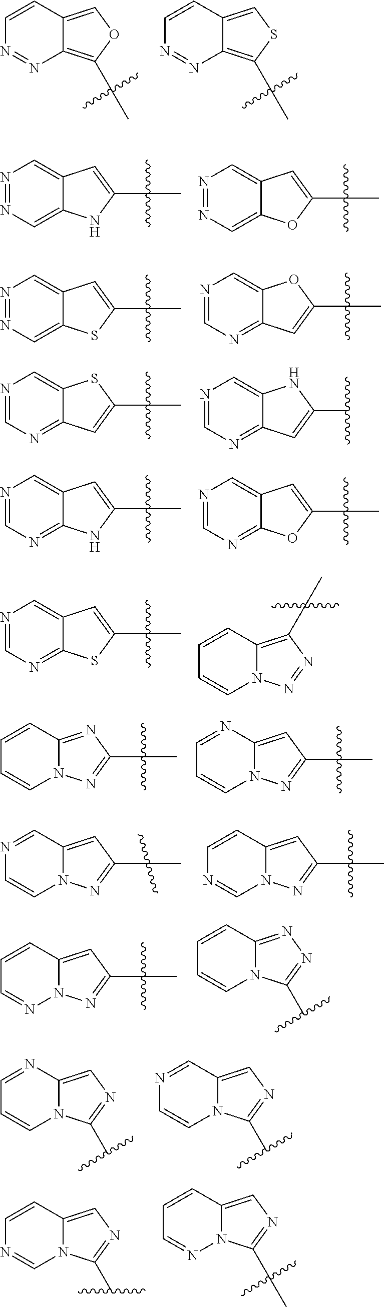

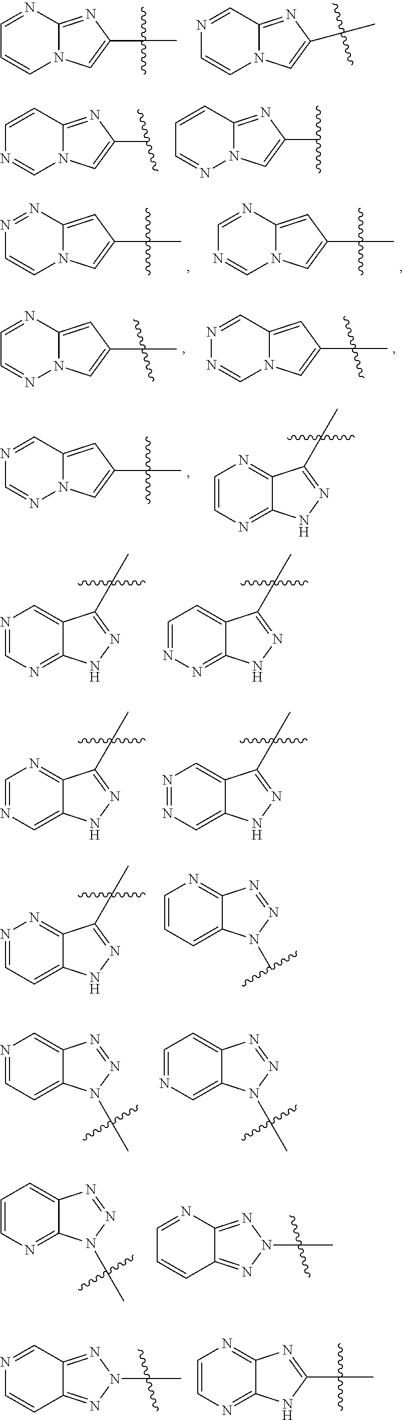

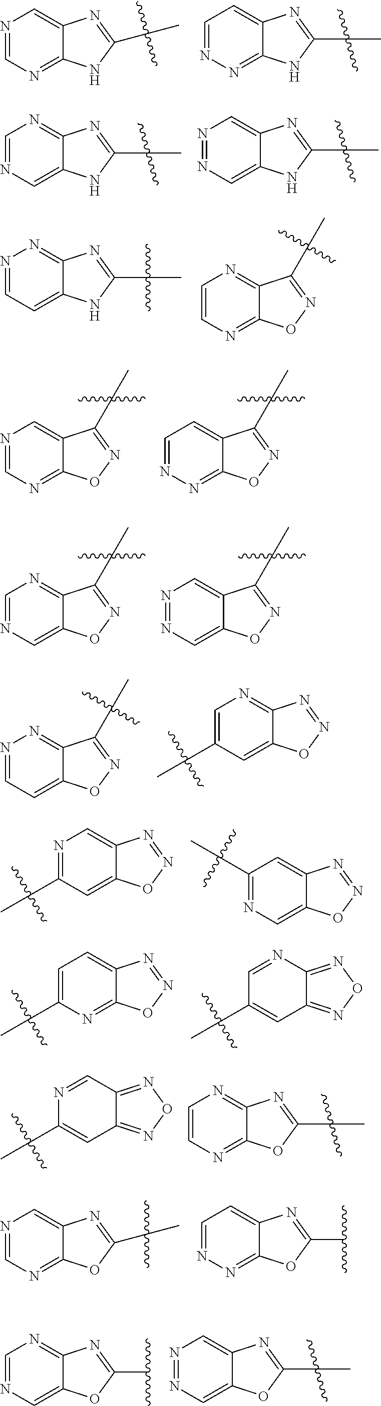

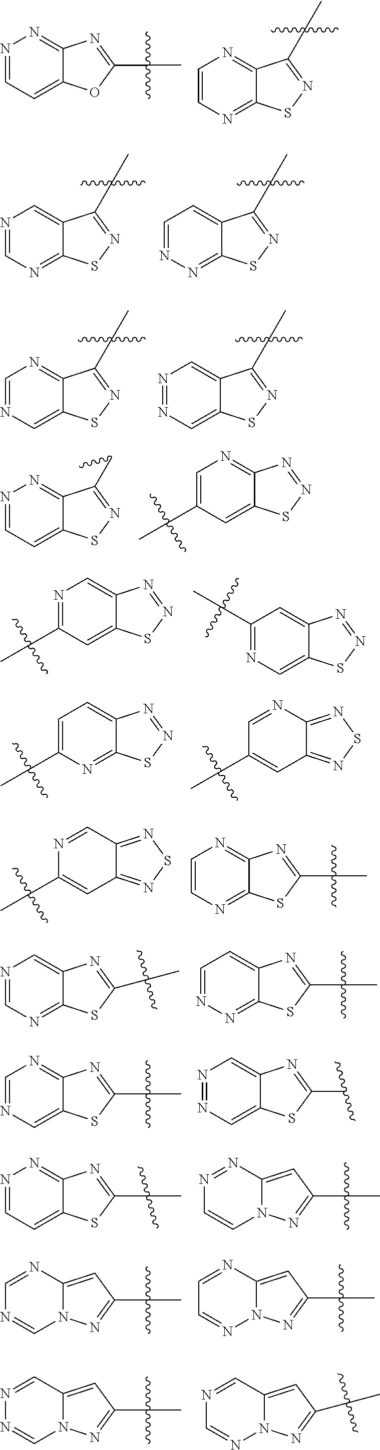

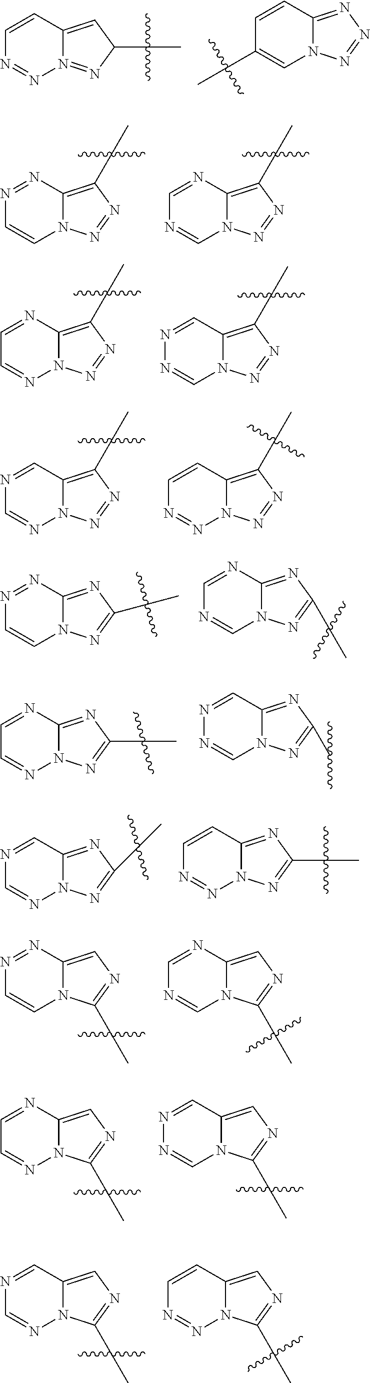

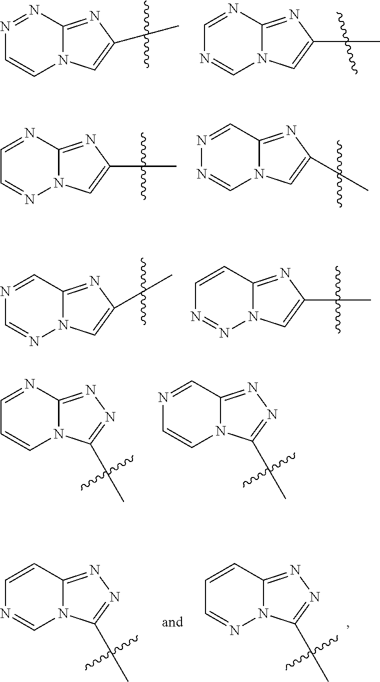

each of which may be optionally substituted with 1, 2, 3, 4, or 5 R^{y} groups as valency permits.

In certain embodiments, Ring Z, e.g., Cy^{A}, Ring A, and the like, is an optionally substituted heterocyclyl (i.e., an optionally substituted dihydroimidazo pyrimidinyl) selected from the group consisting of:

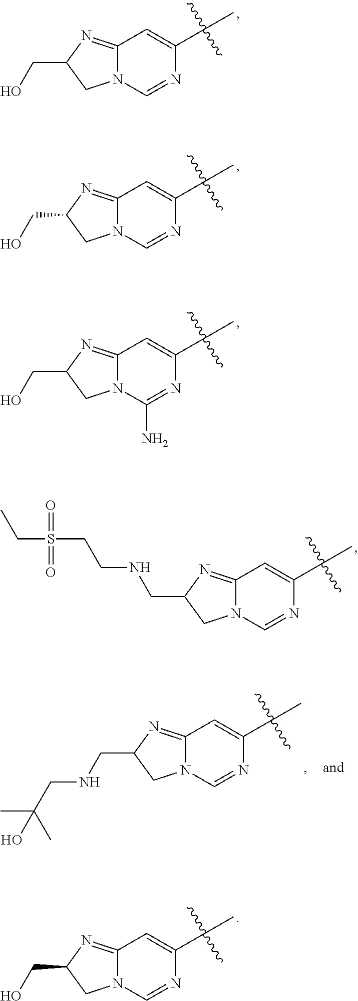 [SMILES: *C1=CC2=NC(CO)CN2C=N1 ; *C1=CC2=N[C@@H](CO)CN2C=N1 ; *C1=CC2=NC(CO)CN2C(N)=N1 ; *C1=CC2=NC(CNCCS(=O)(=O)CC)CN2C=N1 ; *C1=CC2=NC(CNCC(C)(C)O)CN2C=N1 ; *C1=CC2=N[C@H](CO)CN2C=N1]

In certain embodiments, Ring Z, e.g., Ar, Cy^{A}, Ring A, and the like, is selected from the group consisting of:

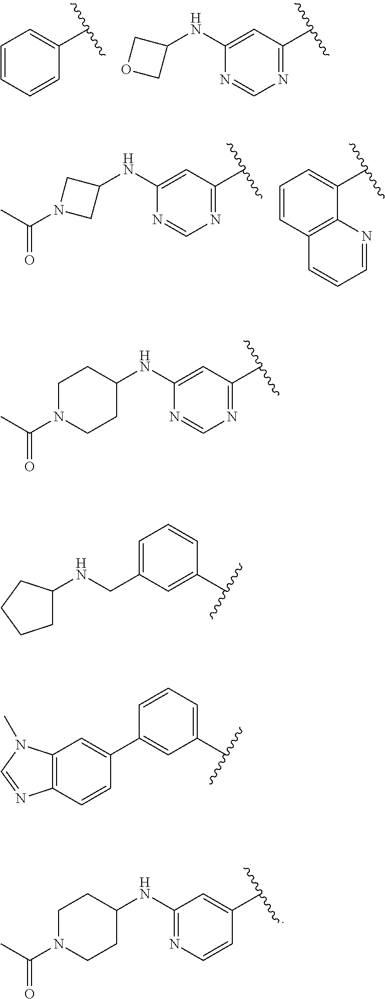

As defined generally above, Cy^{A} is a monocyclic or bicyclic, saturated, partially unsaturated, or aromatic ring having 0-4 heteroatoms independently selected from nitrogen, oxygen, and sulfur, wherein Cy^{A} is substituted with 0, 1, 2, 3, or 4 R^{y} groups. In certain embodiments, Cy^{A} is unsubstituted. In certain embodiments, Cy^{A} is substituted with one or two R^{y} groups. In certain embodiments, Cy^{A} is substituted with one R^{y} group. In certain embodiments, Cy^{A} is substituted with two R^{y} groups. In certain embodiments, Cy^{A} is substituted with three R^{y} groups. In certain embodiments, Cy^{A} is substituted with four R^{y} groups.

In certain embodiments, Cy^{A} is phenyl substituted with 0, 1, 2, 3, or 4 R^{y} groups. In certain embodiments, Cy^{A} is phenyl substituted with one or two R^{y} groups. In certain embodiments, Cy^{A} is unsubstituted phenyl. In certain embodiments, Cy^{A} is phenyl substituted with one R^{y} group. In certain embodiments, Cy^{A} is phenyl substituted with two R′ groups. In certain embodiments, Cy^{A} is phenyl substituted with three R^{y} groups. In certain embodiments, Cy^{A} is phenyl substituted with four R^{y} groups.

In certain embodiments, Cy^{A} is a 5- to 6-membered heteroaryl having 1-3 heteroatoms independently selected from nitrogen, oxygen, and sulfur, and is substituted with 0, 1, 2, 3, or 4 R^{y} groups. In certain embodiments, Cy^{A} is an unsubstituted 5- to 6-membered heteroaryl having 1-3 heteroatoms independently selected from nitrogen, oxygen, and sulfur. In certain embodiments, Cy^{A} is a 5- to 6-membered heteroaryl having 1-3 heteroatoms independently selected from nitrogen, oxygen, and sulfur, and is substituted with one or two R^{y} groups. In certain embodiments, Cy^{A} is a 5- to 6-membered heteroaryl having 1-3 heteroatoms independently selected from nitrogen, oxygen, and sulfur, and is substituted with one R^{y} group. In certain embodiments, Cy^{A} is a 5-membered heteroaryl having 1-3 heteroatoms independently selected from nitrogen, oxygen, and sulfur (e.g., furanyl, thienyl, pyrrolyl, oxazolyl, isoxazolyl, thiazolyl, imidazolyl, pyrazolyl, isothiazolyl, triazolyl, oxadiazolyl, thiadiazolyl), and is substituted with 0, 1, 2, 3, or 4 R^{y} groups. In certain embodiments, Cy^{A} is a 6-membered heteroaryl having 1-3 nitrogens (e.g., pyridyl, pyrimidyl, pyridazinyl, pyrazinyl, triazinyl), and is substituted with 0, 1, 2, 3, or 4 R^{y} groups.

In certain embodiments, Cy^{A} is a bicyclic saturated, partially unsaturated, or aromatic ring having 0-4 heteroatoms independently selected from nitrogen, oxygen, and sulfur, wherein Cy^{A} is substituted with 0, 1, 2, 3, or 4 R^{y} groups. In certain embodiments, Cy^{A} is an 8- to 12-membered bicyclic saturated, partially unsaturated, or aromatic ring having 0-4 heteroatoms independently selected from nitrogen, oxygen, and sulfur, wherein Cy^{A} is substituted with 0, 1, 2, 3, or 4 R^{y} groups. In certain embodiments, Cy^{A} is an unsubstituted bicyclic saturated, partially unsaturated, or aromatic ring having 0-4 heteroatoms independently selected from nitrogen, oxygen, and sulfur. In certain embodiments, Cy^{A} is a bicyclic saturated, partially unsaturated, or aromatic ring having 0-4 heteroatoms independently selected from nitrogen, oxygen, and sulfur, wherein Cy^{A} is substituted with one or two R^{y} groups. In certain embodiments, Cy^{A} is a bicyclic saturated, partially unsaturated, or aromatic ring having 0-4 heteroatoms independently selected from nitrogen, oxygen, and sulfur, wherein Cy^{A} is substituted with one R^{y} group. In certain embodiments, Cy^{A} is a bicyclic saturated, partially unsaturated, or aromatic ring having 0-4 heteroatoms independently selected from nitrogen, oxygen, and sulfur, wherein Cy is substituted with two R^{y} groups. In certain embodiments, Cy^{A} is a bicyclic saturated, partially unsaturated, or aromatic ring having 0-4 heteroatoms independently selected from nitrogen, oxygen, and sulfur, wherein Cy^{A} is substituted with three R^{y} groups. In certain embodiments, Cy^{A} is a bicyclic saturated, partially unsaturated, or aromatic ring having 0-4 heteroatoms independently selected from nitrogen, oxygen, and sulfur, wherein Cy^{A} is substituted with four R^{y} groups.

In certain embodiments, Cy^{A} is an 8- to 10-membered bicyclic heteroaryl having 1-4 heteroatoms independently selected from nitrogen, oxygen, and sulfur, wherein Cy^{A} is substituted with 0, 1, 2, 3, or 4 R^{y} groups. In certain embodiments, Cy^{A} is a 9-membered bicyclic heteroaryl having 1-3 heteroatoms independently selected from nitrogen, oxygen, and sulfur (e.g., indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl), wherein Cy^{A} is substituted with 0, 1, 2, 3, or 4 R^{y} groups. In certain embodiments, Cy^{A} is a 10-membered bicyclic heteroaryl having 1-3 heteroatoms independently selected from nitrogen, oxygen, and sulfur (e.g., naphthyridinyl, quinolinyl, isoquinolinyl, quinoxalinyl, quinazolinyl), wherein Cy^{A} is substituted with 0, 1, 2, 3, or 4 R^{y} groups. In certain embodiments, Cy^{A} is selected from the group consisting of quinoline, benzimidazole, benzopyrazole, quinoxaline, tetrahydroquinoline, tetrahydroisoquinoline, naphthalene, tetrahydronaphthalene, 2,3-dihydrobenzo[b][1,4]dioxine, isoindole, 2H-benzo[b][1,4]oxazin-3 (4H)-one, 3,4-dihydro-2H-benzo[b][1,4]oxazine, and quinoxalin-2(1H)-one, wherein Cy^{A} is substituted with 0, 1, 2, 3, or 4 R^{y} groups.

As defined generally above, each R^{y} is independently selected from the group consisting of halo, —CN, —NO₂, optionally substituted aliphatic, optionally substituted carbocyclyl, optionally substituted phenyl, optionally substituted heterocyclyl, optionally substituted heteroaryl, —OR^{A}, —N(R^{B})₂, —SR^{A}, —C(═O)R^{A}, —C(O)OR^{A}, —C(O)SR^{A}, —C(O)N(R^{B})₂, —C(O)N(R^{B})N(R^{B})₂, —OC(O)R^{A}, —OC(O)N(R^{B})₂, —NR^{B}C(O)R^{A}, —NR^{B}C(O)N(R^{B})₂, —NR^{B}C(O)N(R^{B})N(R^{B})₂, —NR^{B}C(O)OR^{A}, —SC(O)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(O)R^{A}, —OS(O)₂R^{A}, —SO₂R^{A}, —NR^{B}SO₂R^{A}, and —SO₂N(R^{B})₂, wherein R^{A} and R^{B} are described herein. In certain embodiments, each R^{y} is independently selected from the group consisting of halo, —CN, —NO₂, optionally substituted aliphatic, optionally substituted carbocyclyl, optionally substituted phenyl, optionally substituted heterocyclyl, optionally substituted heteroaryl, —OR^{A}, —N(R^{B})₂, —SR^{A}, —C(═O)R^{A}, —C(O)OR^{A}, —C(O)SR^{A}, —C(O)N(R^{B})₂, —OC(O)R^{A}, —NR^{B}C(O)R^{A}, —NR^{B}C(O)N(R^{B})₂, —SC(O)R^{A}, —C(═NR^{B})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(O)R^{A}, —SO₂R^{A}, —NR^{B}SO₂R^{A}, and —SO₂N(R^{B})₂, wherein R^{A} and R^{B} are described herein.

In some embodiments, at least one R^{y} is halo. In certain embodiments, at least one R^{y} is fluoro. In certain embodiments, at least one R^{y} is chloro. In some embodiments, at least one R^{y} is —CN. In some embodiments, at least one R^{y} is —OR^{A}, wherein R^{A} is optionally substituted aliphatic. In some embodiments, at least one R^{y} is —OR^{A}, wherein R^{A} is unsubstituted C₁₋₆ alkyl. In certain embodiments, at least one R^{y} is methoxy, ethoxy, or propoxy. In certain embodiments, at least one R^{y} is methoxy. In some embodiments, at least one R^{y} is —OR^{A}, wherein R^{A} is substituted C₁₋₆ alkyl. In certain embodiments, at least one R^{y} is —OCH₂CH₂N(CH₃)₂. In some embodiments, R^{y} is —OR^{A}. In some embodiments, R^{y} is —OR^{A}, wherein R^{A} is optionally substituted heterocyclyl. In some embodiments, R^{y} is —OR^{A}, wherein R^{A} is optionally substituted heteroaryl. In some embodiments, R^{y} is —OR^{A}, wherein R^{A} is optionally substituted cycloalkyl.

In some embodiments, at least one R^{y} is —N(R^{B})₂. In some embodiments, at least one R^{y} is —N(R^{B})₂, wherein each R^{B} is independently selected from hydrogen or C₁₋₆ alkyl. In some embodiments, at least one R^{y} is —NHR^{B}. In some embodiments, at least one R^{y} is —N(C₁₋₆ alkyl)₂, —NH(C₁₋₆ alkyl), or —NH₂. In certain embodiments, at least one R^{y} is —NH₂. In certain embodiments, at least one R^{y} is —NHCH₃. In certain embodiments, at least one R^{y} is —N(CH₃)₂. In some embodiments, R^{y} is —NHR^{B}, wherein R^{B} is optionally substituted heterocyclyl. In some embodiments, R^{y} is —NHR^{B}, wherein R^{B} is optionally substituted heteroaryl. In some embodiments, R^{y} is —NHR^{B}, wherein R^{B} is optionally substituted cycloalkyl. In some embodiments, R^{y} is —N(R^{B})₂, wherein one R^{B} is optionally substituted heterocyclyl, and the other R^{B} is C₁₋₄ alkyl. In some embodiments, R^{y} is —N(R^{B})₂, wherein one R^{B} is optionally substituted heteroaryl, and the other R^{B} is C₁₋₄ alkyl. In some embodiments, R^{y} is —N(R^{B})₂, wherein one R^{B} is optionally substituted cycloalkyl, and the other R^{B} is C₁₋₄ alkyl.

In some embodiments, at least one R^{y} is optionally substituted aliphatic. In certain embodiments, at least one R^{y} is substituted aliphatic. In certain embodiments, at least one R^{y} is unsubstituted aliphatic. In some embodiments, at least one R^{y} is optionally substituted C₁₋₆ alkyl. In certain embodiments, at least one R^{y} is unsubstituted C₁₋₆ alkyl. In certain embodiments, at least one R^{y} is substituted C₁₋₆ alkyl. In certain embodiments, at least one R^{y} is methyl, ethyl, or propyl. In certain embodiments, at least one R^{y} is methyl. In certain embodiments, at least one R^{y} is —CF₃, CHF₂, or CH₂F. In certain embodiments, at least one R^{y} is C₁₋₆ alkyl substituted with aryl, heteroaryl, or heterocyclyl. In certain embodiments, at least one R^{y} is benzyl. In certain embodiments, at least one R^{y} is —(C₁₋₆ alkyl)-heteroaryl. In certain embodiments, at least one R^{y} is —(C₁₋₆ alkyl)-heterocyclyl. In certain embodiments, at least one R^{y} is —CH₂-heteroaryl. In certain embodiments, at least one R^{y} is —CH₂— heterocyclyl.

In some embodiments, at least one R^{y} is —C(O)N(R^{B})₂. In certain embodiments, at least one R^{y} is —C(O)NHR^{B}. In certain embodiments, at least one R^{y} is —C(O)NH₂. In certain embodiments, at least one R^{y} is —C(O)N(R^{B})₂, wherein the R^{B} groups are taken together with their intervening atoms to form an optionally substituted 5- to 6-membered heterocyclyl. In certain embodiments, at least one R^{y} is —C(O)N(R^{B})₂, wherein the R^{B} groups are taken together with their intervening atoms to form an optionally substituted morpholinyl.

In some embodiments, at least one R^{y} is —SO₂N(R^{B})₂. In certain embodiments, at least one R^{y} is —SO₂NHR^{B}. In certain embodiments, at least one R^{y} is —SO₂NH₂. In certain embodiments, at least one R^{y} is —SO₂N(R^{B})₂, wherein neither R^{B} is hydrogen. In certain embodiments, at least one R^{y} is —SO₂NH(C₁₋₆ alkyl) or —SO₂N(C₁₋₆ alkyl)₂. In certain embodiments, at least one R^{y} is —SO₂N(CH₃)₂. In certain embodiments, at least one R^{y} is —SO₂N(R^{B})₂, wherein the R^{B} groups are taken together with their intervening atoms to form an optionally substituted 5- to 6-membered heterocyclyl. In certain embodiments, at least one R^{y} is —SO₂-morpholinyl. In certain embodiments, at least one R^{y} is —SO₂-piperidinyl, —SO₂— piperazinyl, or —SO₂-piperidinyl.

In some embodiments, at least one R^{y} is —SO₂R^{A}. In some embodiments, at least one R^{y} is —SO₂R^{A}, wherein R^{A} is optionally substituted aliphatic. In some embodiments, at least one R^{y} is —SO₂(C₁₋₆ alkyl). In some embodiments, at least one R^{y} is —SO₂CH₃. In some embodiments, at least one R^{y} is —C(O)R^{A}. In some embodiments, at least one R^{y} is —C(O)R^{A}, wherein R^{A} is optionally substituted aliphatic. In some embodiments, at least one R^{y} is —C(O)(C₁₋₆ alkyl). In some embodiments, at least one R^{y} is —C(O)CH₃.

In some embodiments, at least one R^{y} is —N(R^{B})C(O)R^{A}. In certain embodiments, at least one R^{y} is —NHC(O)R^{A}. In certain embodiments, at least one R^{y} is —NHC(O)(C₁₋₆ alkyl). In certain embodiments, at least one R^{y} is —NHC(O)CH₃.

In some embodiments, at least one R^{y} is —N(R^{B})SO₂R^{A}. In some embodiments, at least one R^{y} is —NHSO₂R^{A}. In some embodiments, at least one R^{y} is —N(C₁₋₆ alkyl)SO₂R^{A} In certain embodiments, at least one R^{y} is —NHSO₂(C₁₋₆ alkyl) or —N(C₁₋₆ alkyl)SO₂(C₁₋₆ alkyl). In certain embodiments, at least one R^{y} is —NHSO₂CH₃. In certain embodiments, at least one R^{y} is —N(CH₃)SO₂CH₃.

In some embodiments, at least one R^{y} is optionally substituted heterocyclyl, optionally substituted carbocyclyl, optionally substituted aryl, or optionally substituted heteroaryl. In certain embodiments, at least one R^{y} is an optionally substituted 5- to 6-membered heterocyclyl having 1-2 heteroatoms independently selected from nitrogen, oxygen, and sulfur. In certain embodiments, at least one R^{y} is an optionally substituted 5-membered heterocyclyl having one heteroatom selected from nitrogen, oxygen, and sulfur. In certain embodiments, at least one R^{y} is optionally substituted pyrrolidinyl. In certain embodiments, at least one R^{y} is pyrroldinyl, hydroxypyrrolidinyl, or methylpyrrolidinyl. In certain embodiments, at least one R^{y} is an optionally substituted 6-membered heterocyclyl having 1-2 heteroatoms independently selected from nitrogen, oxygen, and sulfur. In certain embodiments, at least one R^{y} is an optionally substituted 6-membered heterocyclyl having one heteroatom selected from nitrogen, oxygen, and sulfur. In certain embodiments, at least one R^{y} is optionally substituted piperidinyl. In certain embodiments, at least one R^{y} is an optionally substituted 6-membered heterocyclyl having two heteroatoms independently selected from nitrogen, oxygen, and sulfur. In certain embodiments, at least one R^{y} is optionally substituted piperdinyl, optionally substituted piperazinyl, or optionally substituted morpholinyl. In certain embodiments, at least one R^{y} is morpholinyl, tetrahydropyranyl, piperidinyl, methylpiperidinyl, piperazinyl, methylpiperazinyl, acetylpiperazinyl, methylsulfonylpiperazinyl, aziridinyl, or methylaziridinyl. In some embodiments, at least one R^{y} is an optionally substituted 5- to 6-membered heteroaryl having 1-3 heteroatoms independently selected from nitrogen, oxygen, and sulfur. In certain embodiments, at least one R^{y} is an optionally substituted 5-membered heteroaryl having 1-3 heteroatoms independently selected from nitrogen, oxygen, and sulfur. In certain embodiments, at least one R^{y} is an optionally substituted 5-membered heteroaryl having one heteroatom selected from nitrogen, oxygen, and sulfur. In certain embodiments, at least one R^{y} is an optionally substituted 5-membered heteroaryl having two heteroatoms independently selected from nitrogen, oxygen, and sulfur. In certain embodiments, at least one R^{y} is an optionally substituted 6-membered heteroaryl having 1-3 nitrogens. In certain embodiments, at least one R^{y} is an optionally substituted pyrazolyl. In certain embodiments, at least one R^{y} is an optionally substituted imidazolyl. In certain embodiments, at least one R^{y} is an optionally substituted pyridyl. In certain embodiments, at least one R^{y} is an optionally substituted pyrimidyl. In certain embodiments, at least one R^{y} is pyrazolyl, methylpyrazolyl, imidazolyl, or methylimidazolyl.

In some embodiments, two adjacent R^{y} groups may be taken together with their intervening atoms to form a saturated, partially unsaturated, or aromatic ring having 0-4 heteroatoms independently selected from nitrogen, oxygen, and sulfur. In some embodiments, two adjacent R^{y} groups may be taken together with their intervening atoms to form a saturated carbocyclic ring. In some embodiments, two adjacent R^{y} groups may be taken together with their intervening atoms to form a partially unsaturated carbocyclic ring. In some embodiments, two adjacent R^{y} groups may be taken together with their intervening atoms to form a benzene ring. In some embodiments, two adjacent R^{y} groups may be taken together with their intervening atoms to form a saturated ring having 1-2 heteroatoms independently selected from nitrogen, oxygen, and sulfur. In some embodiments, two adjacent R^{y} groups may be taken together with their intervening atoms to form a partially unsaturated ring having 1-2 heteroatoms independently selected from nitrogen, oxygen, and sulfur. In some embodiments, two adjacent R^{y} groups may be taken together with their intervening atoms to form an aromatic ring having 1-3 heteroatoms independently selected from nitrogen, oxygen, and sulfur.

As defined generally above, Ring C is an optionally substituted, 5- to 12-membered, monocyclic or bicyclic, heterocyclyl or heteroaryl having 1-4 heteroatoms independently selected from nitrogen, oxygen, and sulfur. One of ordinary skill in the art will understand that Ring C comprises an amide or thioamide. In certain embodiments, Ring C is an optionally substituted, 5- to 6-membered, monocyclic heteroaryl having 1-4 heteroatoms independently selected from nitrogen, oxygen, and sulfur. In certain embodiments, Ring C is an optionally substituted, 5- to 7-membered, monocyclic heterocyclyl having 1-4 heteroatoms independently selected from nitrogen, oxygen, and sulfur. In certain embodiments, Ring C is an optionally substituted, 8- to 10-membered, bicyclic heteroaryl having 1-4 heteroatoms independently selected from nitrogen, oxygen, and sulfur. In certain embodiments, Ring C is an optionally substituted, 8- to 12-membered, bicyclic heterocyclyl having 1-4 heteroatoms independently selected from nitrogen, oxygen, and sulfur. In certain embodiments, Ring C is an optionally substituted piperdinone. In certain embodiments, Ring C is an optionally substituted pyridinone. In certain embodiments, Ring C is an optionally substituted piperazinone. In certain embodiments, Ring C is an optionally substituted isoindolinone. In certain embodiments, Ring C is an optionally substituted 2H-benzo[b][1,4]oxazin-3(4H)-one. In some embodiments, Ring C is:

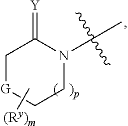

wherein G, R^{y}, m, and p are as described herein.

In certain embodiments, Y is O. In certain embodiments, Y is S.

As defined generally above, G is NR^{2C}, CR^{3C}R^{4C}, O or S. In certain embodiments, G is NR^{2C}. In certain embodiments, G is CR^{3C}R^{4C}. In certain embodiments, G is O. In certain embodiments, G is S.

As defined generally above, R^{2C} is selected from the group consisting of optionally substituted aliphatic, optionally substituted carbocyclyl, optionally substituted aryl, optionally substituted heterocyclyl, optionally substituted heteroaryl, —C(O)R^{A}, —C(O)OR^{A}, —C(O)SR^{A}, —C(O)N(R^{B})₂, —C(═NR^{B})R^{A}, —C(═NR^{B})N(R^{B})₂, —C(═S)R^{A}, —C(═S)N(R^{B})₂, —S(═O)R^{A}, —SO₂R^{A}, and —SO₂N(R^{B})₂. In some embodiments, R^{2C} is optionally substituted aryl. In certain embodiments, R^{2C} is optionally substituted phenyl. In certain embodiments, R^{2C} is unsubstituted phenyl. In certain embodiments, R^{2C} is halophenyl. In certain embodiments, R^{2C} is fluorophenyl. In certain embodiments, R^{2C} is chlorophenyl. In some embodiments, R^{2C} is phenyl substituted with optionally substituted C₁₋₆ alkyl. In some embodiments, R^{2C} is phenyl substituted with optionally substituted C₁₋₃ alkyl. In certain embodiments, R^{2C} is phenyl substituted with methyl. In certain embodiments, R^{2C} is phenyl substituted with —CH₂OH. In some embodiments, R^{2C} is phenyl substituted with a heterocyclic ring. In certain embodiments, R^{2C} is phenyl substituted with morpholinyl. In certain embodiments, R^{2C} is phenyl substituted with tetrahydropyranyl. In some embodiments, R^{2C} is optionally substituted heteroaryl. In certain embodiments, R^{2C} is optionally substituted quinoline. In certain embodiments, R^{2C} is unsubstituted quinoline. In certain embodiments, R^{2C} is substituted quinoline. In certain embodiments, R^{2C} is optionally substituted pyridine. In certain embodiments, R^{2C} is pyridine substituted with a heterocyclic ring. In some embodiments, R^{2C} is optionally substituted aliphatic. In certain embodiments, R^{2C} is unsubstituted aliphatic. In certain embodiments, R^{2C} is —CH₂-aryl. In certain embodiments, R^{2C} is benzyl. In certain embodiments, R^{2C} is —CH₂-heteroaryl. In certain embodiments, R^{2C} is —CH₂-pyridyl. In some embodiments, R^{2C} is —C(═O)R^{A}. In certain embodiments, R^{2C} is —C(═O)R^{A}, wherein R^{A} is optionally substituted aliphatic. In certain embodiments, R² is acetyl. In certain embodiments, R^{2C} is —SO₂R^{A}. In certain embodiments, R^{2C} is —SO₂R^{A}, wherein R^{A} is optionally substituted aliphatic. In certain embodiments, R^{2C} is —SO₂CH₃.

In certain embodiments, R^{2C} is selected from, but is not limited to, any one of the following aryl groups:

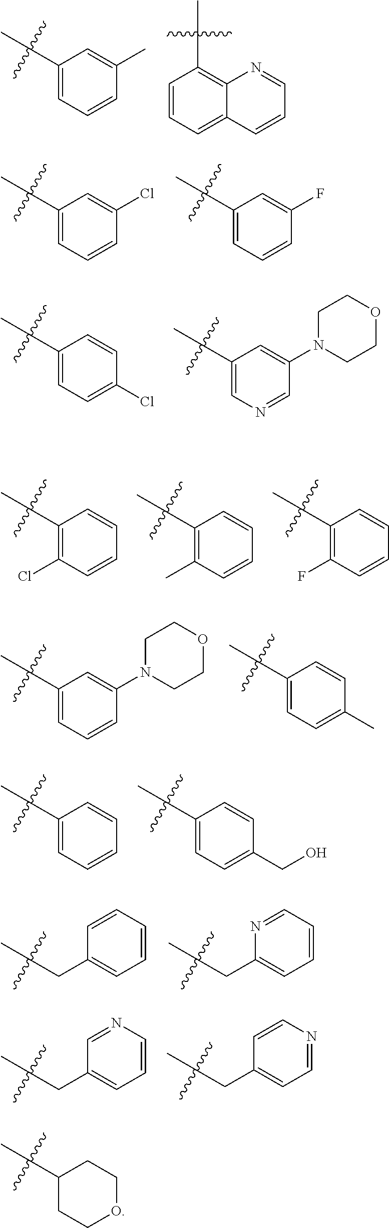

As defined generally above, R^{3C} is selected from the group consisting of hydrogen, halo, optionally substituted aliphatic, optionally substituted carbocyclyl, optionally substituted aryl, optionally substituted heterocyclyl, optionally substituted heteroaryl, —OR^{A}, —N(R^{B})₂, —SR^{A}, —C(═O)R^{A}, —C(O)OR^{A}, —C(O)SR^{A}, —C(O)N(R^{B})₂, —C(O)N(R^{B})N(R^{B})₂, —OC(O)R^{A}, —OC(O)N(R^{B})₂, —NR^{B}C(O)R^{A}, —NR^{B}C(O)N(R^{B})₂, —NR^{B}C(O)N(R^{B})N(R^{B})₂, —NR^{B}C(O)OR^{A}, —SC(O)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(O)R^{A}, —OS(O)₂R^{A}, —SO₂R^{A}, —NR^{B}SO₂R^{A}, and —SO₂N(R^{B})₂. In certain embodiments, R^{3C} is selected from the group consisting of hydrogen, halo, optionally substituted aliphatic, optionally substituted carbocyclyl, optionally substituted aryl, optionally substituted heterocyclyl, optionally substituted heteroaryl, —OR^{A}, —N(R^{B})₂, —SR^{A}, —C(O)R^{A}, —C(O)OR^{A}, —C(O)SR^{A}, —C(O)N(R^{B})₂, —OC(O)R^{A}, —NR^{B}C(═O)R^{A}, —NR^{B}C(═O)N(R^{B})₂, —SC(═O)R^{A}, —C(═NR^{B})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(═O)R^{A}, —SO₂R^{A}, —NR^{B}SO₂R^{A}, and —SO₂N(R^{B})₂.

In certain embodiments, R^{3C} is hydrogen. In some embodiments, R^{3C} is not hydrogen. In some embodiments, R^{3C} is halo. In certain embodiments, R^{3C} is fluoro. In some embodiments, R^{3C} is optionally substituted aliphatic. In certain embodiments, R^{3C} is optionally substituted C₁₋₆ aliphatic. In certain embodiments, R^{3C} is optionally substituted C₁₋₆ alkyl. In certain embodiments, R^{3C} is substituted C₁₋₆ alkyl. In certain embodiments, R^{3C} is —CF₃, —CHF₂, or —CH₂F. In certain embodiments, R^{3C} is unsubstituted C₁₋₆ alkyl. In certain embodiments, R^{3C} is methyl, ethyl, or propyl. In some embodiments, R^{3C} is —CN or —NO₂. In some embodiments, R^{3C} is optionally substituted carbocyclyl, optionally substituted phenyl, optionally substituted heterocyclyl, or optionally substituted heteroaryl. In some embodiments, R^{3C} is —OR^{A}, —N(R^{B})₂, —SR^{A}, —C(═O)R^{A}, —C(O)OR^{A}, —C(O)SR^{A}, —C(O)N(R^{B})₂, —OC(O)R^{A}, —NR^{B}C(O)R^{A}, —NR^{B}C(O)N(R^{B})₂, —SC(O)R^{A}, —C(═NR^{B})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(O)R^{A}, —SO₂R^{A}, —NR^{B}SO₂R^{A}, or —SO₂N(R^{B})₂. In some embodiments, R^{3C} is optionally substituted aryl. In certain embodiments, R^{3C} is optionally substituted phenyl. In certain embodiments, R^{3C} is unsubstituted phenyl. In certain embodiments, R^{3C} is halophenyl. In certain embodiments, R^{3C} is fluorophenyl. In certain embodiments, R^{3C} is chlorophenyl. In some embodiments, R^{3C} is phenyl substituted with optionally substituted C₁₋₆ alkyl. In some embodiments, R^{3C} is phenyl substituted with optionally substituted C₁₋₃ alkyl. In certain embodiments, R^{3C} is phenyl substituted with methyl. In certain embodiments, R^{3C} is phenyl substituted with —CH₂OH. In some embodiments, R^{3C} is phenyl substituted with a heterocyclic ring. In certain embodiments, R^{3C} is phenyl substituted with morpholinyl. In certain embodiments, R^{3C} is phenyl substituted with tetrahydropyranyl. In some embodiments, R^{3C} is optionally substituted heteroaryl. In certain embodiments, R^{3C} is optionally substituted quinoline. In certain embodiments, R^{3C} is unsubstituted quinoline. In certain embodiments, R^{3C} is substituted quinoline. In certain embodiments, R^{3C} is optionally substituted pyridine. In certain embodiments, R^{3C} is pyridine substituted with a heterocyclic ring. In some embodiments, R^{3C} is optionally substituted aliphatic. In certain embodiments, R^{3C} is unsubstituted aliphatic. In certain embodiments, R^{3C} is —CH₂-aryl. In certain embodiments, R^{3C} is benzyl. In certain embodiments, R^{3C} is —CH₂-heteroaryl. In certain embodiments, R^{3C} is —CH₂-pyridyl.

As defined generally above, R^{4C} is selected from the group consisting of hydrogen, halo, or optionally substituted aliphatic. In certain embodiments, R^{4C} is hydrogen. In some embodiments, R^{4C} is not hydrogen. In some embodiments, R^{4C} is halo. In certain embodiments, R^{4C} is fluoro. In some embodiments, R^{4C} is optionally substituted aliphatic. In certain embodiments, R^{4C} is optionally substituted C₁₋₆ aliphatic. In certain embodiments, R^{4C} is optionally substituted C₁₋₆ alkyl. In certain embodiments, R^{4C} is substituted C₁₋₆ alkyl. In certain embodiments, R^{4C} is unsubstituted C₁₋₆ alkyl. In certain embodiments, R^{4C} is methyl, ethyl, or propyl.

As defined generally above, p is 0, 1, or 2. In certain embodiments, p is 0. In certain embodiments, p is 1. In certain embodiments, p is 2.

As defined generally above, L_{D} is the linker L_{B} as defined herein, or L_{D} is —O—, —N(R)—, —C(R^{2A})(R^{3A})—, —O—CR^{2A}R^{3A}, —N(R)—CR^{2A}R^{3A}—, —O—CR^{2A}R^{3A}—O—, —N(R)—CR^{2A}R^{3A}-O, —N(R)—CR^{2A}R^{3A}—N(R)—, —O—CR^{2A}R^{3A}—N(R)—, —CR^{2A}R^{3A}—O—, —CR^{2A}R^{3A}—N(R)—, —O—CR^{2A}R^{3A}—CR⁹R¹⁰—, —N(R)—CR^{2A}R^{3A}—CR⁹R¹⁰—, —CR^{2A}R^{3A}—CR⁹R¹⁰—O—, —CR^{2A}R^{3A}-CR⁹R¹⁰—N(R)—, or —CR^{2A}R^{3A}—CR⁹R¹⁰—. In certain embodiments, L_{D} is —O—, —N(R)—, or —CR^{2A}R^{3A}—, wherein R, R^{2A}, and R^{3A} are as described herein. In certain embodiments, L_{D} is —O—. In some embodiments, L_{D} is —N(R)—. In certain embodiments, L_{D} is —NH—. In certain embodiments, L_{D} is —N(R)—, wherein R is optionally substituted C₁₋₆ aliphatic. In certain embodiments, L_{D} is —N(R)—, wherein R is optionally substituted C₁₋₆ alkyl. In certain embodiments, L_{D} is —N(R)—, wherein R is unsubstituted C₁₋₆ alkyl. In certain embodiments, L_{D} is —N(R)—, wherein R is acetyl. In certain embodiments, L_{D} is —CH₂—O—. In certain embodiments, L_{D} is —CR^{2A}R^{3A}—O—. In certain embodiments, L_{D} is —CR^{2A}R^{3A}—N(R)—. In certain embodiments, L_{D} is —CH₂—NH—.

In certain embodiments, L_{D} is —O—, —N(R)—, —C(R^{2A})(R^{3A})—, —O—CR^{2A}R^{3A}, —N(R)—CR^{2A}R^{3A}—, —O—CR^{2A}R^{3A}—O—, —N(R)—CR^{2A}R^{3A}—O, —N(R)—CR^{2A}R^{3A}—N(R)—, —O—CR^{2A}R^{3A}—N(R)—, —CR^{2A}R^{3A}—N(R), —O—CR^{2A}R^{3A}—CR⁹R¹⁰—, —N(R)—CR^{2A}R^{3A}—CR⁹R¹⁰—, —CR^{2A}R^{3A}—CR⁹R¹⁰—O—, —CR^{2A}R^{3A}—CR⁹R¹⁰—N(R)—, or —CR^{2A}R^{3A}—CR⁹R—, wherein R, R^{2A}, and R^{3A} are as described herein.

In certain embodiments, when L_{D} is —CR^{2A}R^{3A}—O—, R^{2A}, and R^{3A} are not taken together with their intervening atoms to form optionally substituted phenylene. In certain embodiments, when L_{D} is —CR^{2A}R^{3A}—O—, R^{2A}, and R^{3A} are not taken together with their intervening atoms to form phenyl. In certain embodiments, L_{D} is not

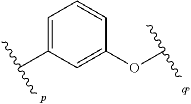 [SMILES: *Oc1cccc(*)c1]

wherein p indicates point of attachment to Ring Z, and q indicates point of attachment to the carbon substituted by R²¹ and R²².

As defined generally above, Ring A is a monocyclic or bicyclic, saturated, partially unsaturated, or aromatic ring having 0-4 heteroatoms independently selected from nitrogen, oxygen, and sulfur. In certain embodiments, Ring A is aromatic. In certain embodiments, Ring A is saturated. In certain embodiments, Ring A is monocyclic. In certain embodiments, Ring A is bicyclic.

In certain embodiments, Ring A is phenyl. In certain embodiments, Ring A is a monocyclic heteroaryl having 1-3 heteroatoms independently selected from nitrogen, oxygen, and sulfur. In certain embodiments, Ring A is a 5- to 6-membered heteroaryl having 1-3 heteroatoms independently selected from nitrogen, oxygen, and sulfur. In certain embodiments, Ring A is a 5-membered heteroaryl having 1-3 heteroatoms independently selected from nitrogen, oxygen, and sulfur (e.g., furanyl, thienyl, pyrrolyl, oxazolyl, isoxazolyl, thiazolyl, imidazolyl, pyrazolyl, isothiazolyl, triazolyl, oxadiazolyl, thiadiazolyl. In certain embodiments, Ring A is a 6-membered heteroaryl having 1-3 nitrogens (e.g., pyridyl, pyrimidyl, pyridazinyl, pyrazinyl, triazinyl). In certain embodiments, Ring A is pyridyl. In certain embodiments, Ring A is pyrimidyl. In certain embodiments, Ring A is pyridazinyl. In some embodiments, Ring A is a carbocyclic ring. In some embodiments, Ring A is a 3- to 8-membered saturated carbocyclic ring. In some embodiments, Ring A is a 3- to 8-membered heterocyclic ring having 1-2 heteroatoms independently selected from nitrogen, oxygen, and sulfur.

In certain embodiments, Ring A is a bicyclic saturated, partially unsaturated, or aromatic ring having 0-4 heteroatoms independently selected from nitrogen, oxygen, and sulfur. In certain embodiments, Ring A is an 8- to 12-membered bicyclic saturated, partially unsaturated, or aromatic ring having 0-4 heteroatoms independently selected from nitrogen, oxygen, and sulfur. In certain embodiments, Ring A is an 8- to 10-membered bicyclic heteroaryl having 1-4 heteroatoms independently selected from nitrogen, oxygen, and sulfur. In certain embodiments, Ring A is a 9-membered bicyclic heteroaryl having 1-3 heteroatoms independently selected from nitrogen, oxygen, and sulfur (e.g., indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl). In certain embodiments, Ring A is a 10-membered bicyclic heteroaryl having 1-3 heteroatoms independently selected from nitrogen, oxygen, and sulfur (e.g., naphthyridinyl, quinolinyl, isoquinolinyl, quinoxalinyl, quinazolinyl. In certain embodiments, Ring A is selected from the group consisting of quinoline, benzimidazole, benzopyrazole, quinoxaline, tetrahydroquinoline, tetrahydroisoquinoline, naphthalene, tetrahydronaphthalene, 2,3-dihydrobenzo[b][1,4]dioxine, isoindole, 2H-benzo[b][1,4]oxazin-3 (4H)-one, 3,4-dihydro-2H-benzo[b][1,4]oxazine, and quinoxalin-2(1H)-one.

In some embodiments, q is 0. In some embodiments, q is 1. In certain embodiments, q is 0 and m is 1. In certain embodiments, q is 0 and m is 2. In certain embodiments, q is 1 and m is 1. In certain embodiments, q is 1 and m is 2.

As defined generally above, L₁ is a bond, —O—, —S—, —N(R)—, —C(O)—, —C(O)N(R)—, —N(R)C(O)N(R)—, —N(R)C(O)—, —N(R)C(O)O—, —OC(O)N(R)—, —SO₂—, —SO₂N(R)—, —N(R)SO₂—, —OC(O)—, —C(O)O—, or an optionally substituted, straight or branched, C₁₋₆ aliphatic chain wherein one, two, or three methylene units of L₁ are optionally and independently replaced by —O—, —S—, —N(R)—, —C(O)—, —C(O)N(R)—, —N(R)C(O)N(R)—, —N(R)C(O)—, —N(R)C(O)O—, —OC(O)N(R)—, —SO₂—, —SO₂N(R)—, —N(R)SO₂—, —OC(O)—, or —C(O)O—. In some embodiments, L₁ is a bond. In some embodiments, L₁ is —O—, —S—, or —N(R)—. In some embodiments, L₁ is —C(O)—, —C(O)N(R)—, or —N(R)C(O)—. In some embodiments, L₁ is a C₁₋₆ aliphatic chain wherein one, two, or three methylene units of L₁ are optionally and independently replaced by —O—, —S—, —N(R)—, —C(O)—, —C(O)N(R)—, —N(R)C(O)N(R)—, —N(R)C(O)—, —N(R)C(O)O—, —OC(O)N(R)—, —SO₂—, —SO₂N(R)—, —N(R)SO₂—, —OC(O)—, or —C(O)O—. In some embodiments, L₁ is a C₁₋₃ aliphatic chain wherein one methylene unit of L₁ is optionally replaced by —O—, —S—, —N(R)—, —C(O)—, —C(O)N(R)—, —N(R)C(O)N(R)—, —N(R)C(O)—, —N(R)C(O)O—, —OC(O)N(R)—, —SO₂—, —SO₂N(R)—, —N(R)SO₂—, —OC(O)—, or —C(O)O—. In some embodiments, L₁ is —CHNH—.

As defined generally above, Cy^{D} is an optionally substituted, monocyclic, bicyclic or tricyclic, saturated, partially unsaturated, or aromatic ring having 0-4 heteroatoms independently selected from nitrogen, oxygen, and sulfur. In certain embodiments, Cy^{D} is aromatic. In certain embodiments, Cy^{D} is saturated. In certain embodiments, Cy^{D} is monocyclic. In certain embodiments, Cy^{D} is bicyclic. In certain embodiments, Cy^{D} is tricyclic.

In certain embodiments, Cy^{D} is optionally substituted phenyl. In certain embodiments, Cy^{D} is an optionally substituted 5- to 6-membered heteroaryl having 1-3 heteroatoms independently selected from nitrogen, oxygen, and sulfur. In certain embodiments, Cy^{D} is an optionally substituted 5-membered heteroaryl having 1-3 heteroatoms independently selected from nitrogen, oxygen, and sulfur (e.g., furanyl, thienyl, pyrrolyl, oxazolyl, isoxazolyl, thiazolyl, imidazolyl, pyrazolyl, isothiazolyl, triazolyl, oxadiazolyl, thiadiazolyl. In certain embodiments, Cy^{D} is an optionally substituted 6-membered heteroaryl having 1-3 nitrogens (e.g., pyridyl, pyrimidyl, pyridazinyl, pyrazinyl, triazinyl). In certain embodiments, Cy^{D} is optionally substituted pyrazole, optionally substituted pyridyl, or optionally substituted pyrimidyl. In some embodiments, Cy^{D} is an optionally substituted carbocyclic ring. In some embodiments, Cy^{D} is an optionally substituted 3- to 8-membered saturated carbocyclic ring. In some embodiments, Cy^{D} is an optionally substituted 3- to 8-membered heterocyclic ring having 1-2 heteroatoms independently selected from nitrogen, oxygen, and sulfur.

In certain embodiments, Cy^{D} is an optionally substituted bicyclic saturated, partially unsaturated, or aromatic ring having 0-4 heteroatoms independently selected from nitrogen, oxygen, and sulfur. In certain embodiments, Cy^{D} is an optionally substituted 8- to 12-membered bicyclic saturated, partially unsaturated, or aromatic ring having 0-4 heteroatoms independently selected from nitrogen, oxygen, and sulfur. In certain embodiments, Cy^{D} is an optionally substituted 8- to 10-membered bicyclic heteroaryl having 1-4 heteroatoms independently selected from nitrogen, oxygen, and sulfur. In certain embodiments, Cy^{D} is an optionally substituted 9- to 10-membered bicyclic heteroaryl having 1-4 heteroatoms independently selected from nitrogen, oxygen, and sulfur. In certain embodiments, Cy^{D} is an optionally substituted 9-membered bicyclic heteroaryl having 1-3 heteroatoms independently selected from nitrogen, oxygen, and sulfur (e.g., indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl). In certain embodiments, Cy^{D} is an optionally substituted 10-membered bicyclic heteroaryl having 1-3 heteroatoms independently selected from nitrogen, oxygen, and sulfur (e.g., naphthyridinyl, quinolinyl, isoquinolinyl, quinoxalinyl, quinazolinyl. In certain embodiments, Cy^{D} is optionally substituted indazole, optionally substituted quinoline, optionally substituted benzimidazole, optionally substituted benzothiazole, optionally substituted deazapurine, optionally substituted indole, optionally substituted purine, optionally substituted pyrazolopyridine, optionally substituted pyrrolopyridine, optionally substituted pyrroloprimidine, optionally substituted imidazopyridine, or optionally substituted imidazopyridine.

As defined generally above, R⁹ and R¹⁰ are independently selected from the group consisting of hydrogen, halo, —CN, —NO₂, optionally substituted aliphatic, optionally substituted carbocyclyl, optionally substituted phenyl, optionally substituted heterocyclyl, optionally substituted heteroaryl, —OR^{A}, —N(R^{B})₂, —SR^{A}, —C(═O)R^{A}, —C(O)OR^{A}, —C(O)SR^{A}, —C(O)N(R^{B})₂, —C(O)N(R^{B})N(R^{B})₂, —OC(O)R^{A}, —OC(O)N(R^{B})₂, —NR^{B}C(O)R^{A}, —NR^{B}C(O)N(R^{B})₂, —NR^{B}C(O)N(R^{B})N(R^{B})₂, —NR^{B}C(O)OR^{A}, —SC(O)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(O)R^{A}, —OS(O)₂R^{A}, —SO₂R^{A}, —NR^{B}SO₂R^{A}, and —SO₂N(R^{B})₂; or R⁹ and R¹⁰ are taken together with their intervening atoms to form an optionally substituted carbocyclic or heterocyclic ring. In certain embodiments, R⁹ and R¹⁰ are independently selected from the group consisting of hydrogen, halo, —CN, —NO₂, optionally substituted aliphatic, optionally substituted carbocyclyl, optionally substituted phenyl, optionally substituted heterocyclyl, optionally substituted heteroaryl, —OR^{A}, —N(R^{B})₂, —SR^{A}, —C(═O)R^{A}, —C(O)OR^{A}, —C(O)SR^{A}, —C(O)N(R^{B})₂, —OC(O)R^{A}, —NR^{B}C(O)R^{A}, —NR^{B}C(O)N(R^{B})₂, —SC(O)R^{A}, —C(═NR^{B})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(O)R^{A}, —SO₂R^{A}, —NR^{B}SO₂R^{A}, and —SO₂N(R^{B})₂; or R⁹ and R¹⁰ are taken together with their intervening atoms to form an optionally substituted carbocyclic or heterocyclic ring.

In certain embodiments, R⁹ is hydrogen. In some embodiments, R⁹ is not hydrogen. In some embodiments, R⁹ is halo. In certain embodiments, R⁹ is fluoro. In some embodiments, R⁹ is optionally substituted aliphatic. In certain embodiments, R⁹ is optionally substituted C₁₋₆ aliphatic. In certain embodiments, R⁹ is optionally substituted C₁₋₆ alkyl. In certain embodiments, R⁹ is substituted C₁₋₆ alkyl. In certain embodiments, R⁹ is —CF₃, CHF₂, or CH₂F. In certain embodiments, R⁹ is unsubstituted C₁₋₆ alkyl. In certain embodiments, R⁹ is methyl, ethyl, or propyl. In some embodiments, R⁹ is —CN or —NO₂. In some embodiments, R⁹ is optionally substituted carbocyclyl, optionally substituted phenyl, optionally substituted heterocyclyl, or optionally substituted heteroaryl. In some embodiments, R⁹ is —OR^{A}, —N(R^{B})₂, —SR^{A}, —C(═O)R^{A}, —C(O)OR^{A}, —C(O)SR^{A}, —C(O)N(R^{B})₂, —OC(O)R^{A}, —NR^{B}C(O)R^{A}, —NR^{B}C(O)N(R^{B})₂, —SC(O)R^{A}, —C(═NR^{B})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(O)R^{A}, —SO₂R^{A}, —NR^{B}SO₂R^{A}, or —SO₂N(R^{B})₂. In certain embodiments, R⁹ is —N(R^{B})₂. In certain embodiments, R⁹ is —NHR^{B}. In certain embodiments, R⁹ is —NH₂. In certain embodiments, R⁹ is —OR^{A}. In certain embodiments, R⁹ is —OH.

In certain embodiments, R¹⁰ is hydrogen. In some embodiments, R¹⁰ is not hydrogen. In some embodiments, R¹⁰ is halo. In certain embodiments, R¹⁰ is fluoro. In some embodiments, R¹⁰ is optionally substituted aliphatic. In certain embodiments, R¹⁰ is optionally substituted C₁₋₆ aliphatic. In certain embodiments, R¹⁰ is optionally substituted C₁₋₆ alkyl. In certain embodiments, R¹⁰ is substituted C₁₋₆ alkyl. In certain embodiments, R¹⁰ is —CF₃, CHF₂, or CH₂F. In certain embodiments, R¹⁰ is unsubstituted C₁₋₆ alkyl. In certain embodiments, R¹⁰ is methyl, ethyl, or propyl. In some embodiments, R¹⁰ is —CN or —NO₂. In some embodiments, R¹⁰ is optionally substituted carbocyclyl, optionally substituted phenyl, optionally substituted heterocyclyl, or optionally substituted heteroaryl. In some embodiments, R¹⁰ is —OR^{A}, —N(R^{B})₂, —SR^{A}, —C(═O)R^{A}, —C(O)OR^{A}, —C(O)SR^{A}, —C(O)N(R^{B})₂, —OC(O)R^{A}, —NR^{B}C(O)R^{A}, —NR^{B}C(O)N(R^{B})₂, —SC(O)R^{A}, —C(═NR^{B})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(O)R^{A}, —SO₂R^{A}, —NR^{B}SO₂R^{A}, or —SO₂N(R^{B})₂. In certain embodiments, R¹⁰ is —N(R^{B})₂. In certain embodiments, R¹⁰ is —NHR^{B}. In certain embodiments, R¹⁰ is —NH₂. In certain embodiments, R¹⁰ is —OR^{A}. In certain embodiments, R¹⁰ is —OH.

In some embodiments, R⁹ and R¹⁰ are the same. In some embodiments, R⁹ and R¹⁰ are different. In some embodiments, R⁹ and R¹⁰ are each hydrogen. In some embodiments, R⁹ is hydrogen and R¹⁰ is not hydrogen. In some embodiments, R⁹ is hydrogen and R¹⁰ is optionally substituted aliphatic. In some embodiments, R⁹ is hydrogen and R¹⁰ is C₁₋₆ alkyl. In some embodiments, R⁹ is hydrogen and R¹⁰ is methyl. In some embodiments, R⁹ is hydrogen and R¹⁰ is ethyl or propyl. In certain embodiments, R⁹ and hydrogen and R¹⁰ is —CF₃, CHF₂, or CH₂F. In some embodiments, R⁹ is hydrogen and R¹⁰ is —N(R^{B})₂ or —OR^{A}. In some embodiments, R⁹ is hydrogen and R¹⁰ is —NH₂. In some embodiments, R⁹ is hydrogen and R¹⁰ is —OH. In some embodiments, R⁹ and R¹⁰ are not hydrogen. In some embodiments, R⁹ and R¹⁰ are independently optionally substituted aliphatic. In some embodiments, R⁹ and R¹⁰ are methyl. In some embodiments, R⁹ and R¹⁰ are taken together with their intervening atoms to form an optionally substituted carbocyclic or heterocyclic ring.

As defined generally above, each R^{x} is independently selected from the group consisting of halo, —CN, optionally substituted aliphatic, —OR′, and —N(R″)₂. In certain embodiments, at least one R^{x} is halo. In certain embodiments, at least one R^{x} is fluoro. In certain embodiments, at least one R^{x} is —CN. In certain embodiments, at least one R^{x} is optionally substituted aliphatic. In certain embodiments, at least one R^{x} is optionally substituted C₁₋₆ alkyl. In certain embodiments, at least one R^{x} is methyl. In certain embodiments, at least one R^{x} is —CF₃. In certain embodiments, at least one R^{x} is optionally substituted aryl. In certain embodiments, at least one R^{x} is phenyl. In certain embodiments, only one R^{x} is phenyl. In certain embodiments, at least one R^{x} is —OR′. In certain embodiments, R^{x} is not —OR′. In certain embodiments, at least one R^{x} is —OCH₃. In certain embodiments, R^{x} is not —OCH₃. In certain embodiments, at least one R^{x} is —N(R″)₂, wherein each instance of R″ is independently hydrogen or optionally substituted aliphatic. In certain embodiments, at least one R^{x} is —NHR″, wherein R″ is independently hydrogen or optionally substituted aliphatic. In certain embodiments, at least one R^{x} is —NH₂. In certain embodiments, at least one R^{x} is —NHR″, wherein R″ is optionally substituted alkyl. In certain embodiments, R^{x} is not —N(R″)₂.

As defined generally above, n is 0, 1, 2, 3, 4, 5, 6, 7, or 8. In certain embodiments, n is 0. In certain embodiments, n is 1. In certain embodiments, n is 2.

As defined generally above, k is 0, 1, 2, 3, or 4. In some embodiments, k is 0. In some embodiments, k is 1. In some embodiments, k is 2.

As defined generally above, X₁, X₂, X₃, and X₄ are independently selected from the group consisting of N, CH, and CR^{y}, provided that at least one of X₂, X₃, and X₄ is not N.

In certain embodiments, X₁ is N. In certain embodiments, X₁ is CH or CR^{y}. In certain embodiments, X₂ is N. In certain embodiments, X₂ is CH or CR^{y}. In certain embodiments, X₃ is N. In certain embodiments, X₃ is CH or CR^{y}. In certain embodiments, X₄ is N. In certain embodiments, X₄ is CH or CR^{y}.

In certain embodiments, each of X₁ and X₂ is N, and each of X₃ and X₄ is independently CH or CR^{y}. In certain embodiments, each of X₁ and X₃ is N, and each of X₂ and X₄ is independently CH or CR^{y}. In certain embodiments, each of X₁ and X₄ is N, and each of X₂ and X₃ is independently CH or CR^{y}. In certain embodiments, each of X₂ and X₄ is N, and each of X₁ and X₃ is independently CH or CR^{y}. In certain embodiments, each of X₂ and X₃ is N, and each of X₁ and X₄ is independently CH or CR^{y}. In certain embodiments, each of X₃ and X₄ is N, and each of X₁ and X₂ is independently CH or CR^{y}.

As generally defined above, R^{A1} and R^{A2} are independently hydrogen, substituted or unsubstituted C₁₋₃ alkyl, substituted or unsubstituted acyl, or a nitrogen protecting group. In some embodiments, R^{A1} is hydrogen. In some embodiments, R^{A1} is substituted or unsubstituted C₁₋₃ alkyl. In some embodiments, R^{A1} is unsubstituted C₁₋₃ alkyl. In some embodiments, R^{A1} is methyl, ethyl, n-propyl, or isopropyl. In some embodiments, R^{A1} is substituted C₁₋₃ alkyl. In some embodiments, R^{A1} is —CF₃, —CHF₂, —CH₂F, or —CH(CF₃)CH₃. In some embodiments, R^{A1} is substituted or unsubstituted acyl. In some embodiments, R^{A1} is acetyl. In some embodiments, R^{A1} is a nitrogen protecting group. In some embodiments, R^{A1} is CH₃SO₂—. In some embodiments, R^{A2} is hydrogen. In some embodiments, R^{A2} is substituted or unsubstituted C₁₋₃ alkyl. In some embodiments, R^{A2} is unsubstituted C₁₋₃ alkyl. In some embodiments, R^{A2} is methyl, ethyl, n-propyl, or isopropyl. In some embodiments, R^{A2} is substituted C₁₋₃ alkyl. In some embodiments, R^{A2} is —CF₃, —CHF₂, —CH₂F, or —CH(CF₃)CH₃. In some embodiments, R^{A2} is substituted or unsubstituted acyl. In some embodiments, R^{A2} is acetyl. In some embodiments, R^{A2} is a nitrogen protecting group. In some embodiments, R^{A2} is CH₃SO₂—. In some embodiments, R^{A1} is hydrogen, and R^{A2} is hydrogen. In some embodiments, R^{A1} is hydrogen, and R^{A2} is substituted or unsubstituted C₁₋₃ alkyl. In some embodiments, R^{A1} is hydrogen, and R^{A2} is methyl, ethyl, n-propyl, or isopropyl. In some embodiments, R^{A1} is hydrogen, and R^{A2} is —CF₃, —CHF₂, —CH₂F, or —CH(CF₃)CH₃. In some embodiments, R^{A1} is hydrogen, and R^{A2} is substituted or unsubstituted acyl. In some embodiments, R^{A1} is hydrogen, and R^{A2} is acetyl. In some embodiments, R^{A1} is hydrogen, and R^{A2} is a nitrogen protecting group. In some embodiments, R^{A1} is hydrogen and R^{A2} is CH₃SO₂—. In some embodiments, R^{A1} is substituted or unsubstituted C₁₋₃ alkyl, and R^{A2} is substituted or unsubstituted C₁₋₃ alkyl. In some embodiments, R^{A1} is substituted or unsubstituted C₁₋₃ alkyl, and R^{A2} is methyl. In some embodiments, R^{A1} is substituted or unsubstituted C₁₋₃ alkyl, and R^{A2} is ethyl. In some embodiments, R^{A1} is substituted or unsubstituted C₁₋₃ alkyl, and R^{A2} is n-propyl. In some embodiments, R^{A1} is substituted or unsubstituted C₁₋₃ alkyl, and R^{A2} is isopropyl. In some embodiments, R^{A1} is substituted or unsubstituted C₁₋₃ alkyl, and R^{A2} is substituted or unsubstituted acyl. In some embodiments, R^{A1} is substituted or unsubstituted C₁₋₃ alkyl, and R^{A2} is a nitrogen protecting group. In some embodiments, R^{A1} is methyl, and R^{A2} is substituted or unsubstituted C₁₋₃ alkyl. In some embodiments, R^{A1} is methyl, and R^{A2} is methyl. In some embodiments, R^{A1} is methyl, and R^{A2} is ethyl. In some embodiments, R^{A1} is methyl, and R^{A2} is n-propyl. In some embodiments, R^{A1} is methyl, and R^{A2} is isopropyl. In some embodiments, R^{A1} is methyl, and R^{A2} is substituted or unsubstituted acyl. In some embodiments, R^{A1} is methyl, and R^{A2} is a nitrogen protecting group. In some embodiments, R^{A1} is ethyl, and R^{A2} is substituted or unsubstituted C₁₋₃ alkyl. In some embodiments, R^{A1} is ethyl, and R^{A2} is methyl. In some embodiments, R^{A1} is ethyl, and R^{A2} is ethyl. In some embodiments, R^{A1} is ethyl, and R^{A2} is n-propyl. In some embodiments, R^{A1} is ethyl, and R^{A2} is isopropyl. In some embodiments, R^{A1} is ethyl, and R^{A2} is substituted or unsubstituted acyl. In some embodiments, R^{A1} is ethyl, and R^{A2} is a nitrogen protecting group. In some embodiments, R^{A1} is n-propyl, and R^{A2} is substituted or unsubstituted C₁₋₃ alkyl. In some embodiments, R^{A1} is n-propyl, and R^{A2} is methyl. In some embodiments, R^{A1} is n-propyl, and R^{A2} is ethyl. In some embodiments, R^{A1} is n-propyl, and R^{A2} is n-propyl. In some embodiments, R^{A1} is n-propyl and R^{A2} is isopropyl. In some embodiments, R^{A1} is n-propyl, and R^{A2} is substituted or unsubstituted acyl. In some embodiments, R^{A1} is n-propyl and R^{A2} is a nitrogen protecting group. In some embodiments, R^{A1} is isopropyl and R^{A2} is substituted or unsubstituted C₁₋₃ alkyl. In some embodiments, R^{A1} is isopropyl and R^{A2} is methyl. In some embodiments, R^{A1} is isopropyl and R^{A2} is ethyl. In some embodiments, R^{A1} is isopropyl, and R^{A2} is n-propyl. In some embodiments, R^{A1} is isopropyl, and R^{A2} is isopropyl. In some embodiments, R^{A1} is isopropyl, and R^{A2} is substituted or unsubstituted acyl. In some embodiments, R^{A1} is isopropyl, and R^{A2} is a nitrogen protecting group. In some embodiments, R^{A1} is substituted or unsubstituted acyl, and R^{A2} is substituted or unsubstituted C₁₋₃ alkyl. In some embodiments, R^{A1} is a nitrogen protecting group, and R^{A2} is substituted or unsubstituted C₁₋₃ alkyl. In some embodiments, R^{A1} is a nitrogen protecting group and R^{A2} is methyl. In some embodiments, R^{A1} is a nitrogen protecting group, and R^{A2} is ethyl. In some embodiments, R^{A1} is a nitrogen protecting group, and R^{A2} is n-propyl. In some embodiments, R^{A1} is a nitrogen protecting group, and R^{A2} is isopropyl. In some embodiments, R^{A1} is a nitrogen protecting group, and R^{A2} is a nitrogen protecting group.

As generally defined above, R^{A1} and R^{A2} can be taken together with the intervening nitrogen atom to form a substituted or unsubstituted 3-6 membered heterocyclic ring. In certain embodiments, R^{A1} and R^{A2} can be taken together with the intervening nitrogen atom to form a substituted or unsubstituted azetidine. In certain embodiments, R^{A1} and R^{A2} can be taken together with the intervening nitrogen atom to form a substituted or unsubstituted pyrrolidine. In certain embodiments, R^{A1} and R^{A2} can be taken together with the intervening nitrogen atom to form a substituted or unsubstituted piperidine. In certain embodiments, R^{A1} and R^{A2} can be taken together with the intervening nitrogen atom to form a substituted or unsubstituted piperazine. In certain embodiments, R^{A1} and R^{A2} can be taken together with the intervening nitrogen atom to form a substituted or unsubstituted morpholine.

Various combinations of certain above-described embodiments are further envisioned herein.

For example, in certain embodiments of formula (A-V^{D}), wherein the right hand ring system of formula (x) is a bicyclic ring system of formula (i), provided is a compound of formula (A-V^{D}-i):

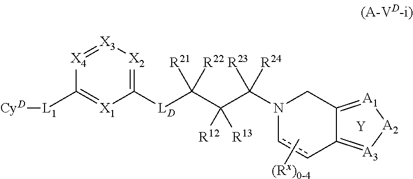

or a pharmaceutically acceptable salt thereof. In certain embodiments, Ring Y does not comprise any R^{x} substituents. In certain embodiments, the heterocyclic ring fused to Ring Y does not comprise any R^{x} substituents, i.e., —(R^{x})₀₋₄ is —(R^{x})₀ and may be depicted absent. In certain embodiments, A₃ is N and A₁ is CH or CR^{x}. In certain embodiments, A₁ is N and A₃ is CH or CR^{x}. In certain embodiments, A₂ is NH or NR^{x}. In certain embodiments, A₃ is N, A₁ is CH or CR^{x}, and A₂ is NH or NR^{x}. In certain embodiments, A₁ is N, A₃ is CH or CR^{x}, and A₂ is NH or NR^{x}. In certain embodiments, R^{x} is optionally substituted alkyl (e.g., —CH₃ or —CF₃) or optionally substituted aryl (e.g., optionally substituted phenyl). In certain embodiments, R^{x} attached to a nitrogen atom on Ring Y is optionally substituted alkyl, e.g., —CH₃. In certain embodiments, each of R²¹, R²², R²³, and R²⁴ is hydrogen. In certain embodiments, R¹ is hydrogen. In some embodiments, carbon attached to —OR¹ has (S)-stereochemistry. In some embodiments, carbon attached to —OR¹ has (R)-stereochemistry. In certain embodiments, X₁ is CH. In certain embodiments, X₂ is N. In certain embodiments, X₃ is CH. In certain embodiments, X₄ is N. In certain embodiments, X₂ and X₄ is N. In certain embodiments, X₁ and X₃ are CH. In certain embodiments, X₂ and X₄ is N and X₁ and X₃ are CH. In certain embodiments, each of X₁, X₂, X₃, and X₄ is CH. In certain embodiments, L₁ is a bond. In certain embodiments, L₁ is NH. In certain embodiments, L₁ is —N(H)CH₂—. In certain embodiments, Cy^{D} is an optionally substituted heterocyclyl (e.g., optionally substituted oxetanyl, optionally substituted azetidinyl, optionally substituted piperidinyl). In certain embodiments, Cy^{D} is an optionally substituted carbocyclyl (e.g., optionally substituted cyclopentyl). In certain embodiments, Cy^{D} is an optionally substituted heteroaryl (e.g., optionally substituted benzoimidazolyl). In certain embodiments, L_{D} is a linker group L_{B}, e.g., L_{D} is —C(O)N(R)—. In certain embodiments, L_{D} is —O—. In certain embodiments, [symbol] represents a single bond.

In certain embodiments of formula (A-V^{D}-i), wherein R¹² is hydrogen and R¹³ is —OR¹, provided is a compound of formula (V^{D}-i-a):

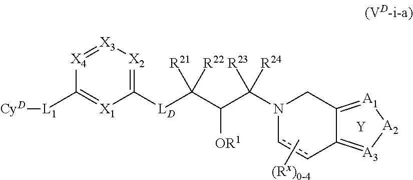

or a pharmaceutically acceptable salt thereof.

In certain embodiments of formula (V^{D}-i-a), wherein R²¹-R²⁴ is hydrogen, the heterocyclic ring fused to Ring Y does not comprise any R^{x} substituents, and [symbol] represents a single bond, provided is a compound of formula (V^{D}-i-b) or (V^{D}-i-c):

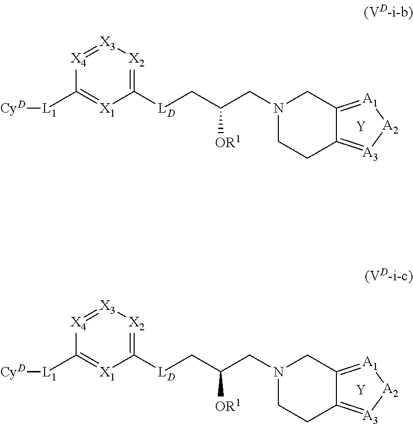

or a pharmaceutically acceptable salt thereof.

In certain embodiments of formula (A-V^{D}), wherein the right hand ring system of formula (x) is a bicyclic ring system of formula (ii), provided is a compound of formula (A-V^{D}-ii):

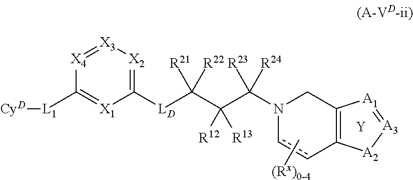

or a pharmaceutically acceptable salt thereof. In certain embodiments, Ring Y does not comprise any R^{x} substituents. In certain embodiments, the heterocyclic ring fused to Ring Y does not comprise any R^{x} substituents, i.e., —(R^{x})₀₋₄ is —(R^{x})₀ and may be depicted absent. In certain embodiments, A₂ is S. In certain embodiments, A₂ is NH or NR^{x}. In certain embodiments, A₁ is CH or CR^{x}. In certain embodiments, A₁ is N. In certain embodiments A₂ is S, and A₁ and A₃ are CH or CR^{x}. In certain embodiments A₂ is S, and A₁ and A₃ are CH. In certain embodiments, A₁ is N and A₂ is NH or NR^{x}. In certain embodiments, A₁ is CH or CR^{x} and A₂ is NH or NR^{x}. In certain embodiments, A₃ is CH or CR^{x}. In certain embodiments, A₃ is N. In certain embodiments, A₃ is N and A₂ is NH or NR^{x}. In certain embodiments, A₃ is CH or CR^{x} and A₂ is NH or NR^{x}. In certain embodiments A₂ is NH or NR^{x}, and A₁ and A₃ are CH or CR^{x}. In certain embodiments A₂ is NH or NR^{x}, A₁ is CR^{x}, and A₃ is CH. In certain embodiments A₂ is NH or NR^{x}, A₁ is CH, and A₃ is CR^{x}. In certain embodiments A₂ is NH or NR^{x}, A₁ is N, and A₃ is CH or CR^{x}. In certain embodiments A₂ is NH or NR^{x}, A₁ is N, and A₃ is CH. In certain embodiments A₂ is NH or NR^{x}, A₁ is N, and A₃ is CR^{x}. In certain embodiments A₂ is NH or NR^{x}, A₃ is N, and A₁ is CH or CR^{x}. In certain embodiments A₂ is NH or NR^{x}, A₃ is N, and A₁ is CH. In certain embodiments A₂ is NH or NR^{x}, A₃ is N, and A₁ is CR^{x}. In certain embodiments, R^{x} is optionally substituted alkyl (e.g., —CH₃ or —CF₃) or optionally substituted aryl (e.g., optionally substituted phenyl). In certain embodiments, R^{x} attached to a nitrogen atom on Ring Y is optionally substituted alkyl, e.g., —CH₃. In certain embodiments, each of R²¹, R²², R²³, and R²⁴ is hydrogen. In certain embodiments, R¹ is hydrogen. In some embodiments, carbon attached to —OR¹ has (S)-stereochemistry. In some embodiments, carbon attached to —OR¹ has (R)-stereochemistry. In certain embodiments, X₁ is CH. In certain embodiments, X₂ is N. In certain embodiments, X₃ is CH. In certain embodiments, X₄ is N. In certain embodiments, X₂ and X₄ is N. In certain embodiments, X₁ and X₃ are CH. In certain embodiments, X₂ and X₄ is N and X₁ and X₃ are CH. In certain embodiments, each of X₁, X₂, X₃, and X₄ is CH. In certain embodiments, L₁ is a bond. In certain embodiments, L₁ is NH. In certain embodiments, L₁ is —N(H)CH₂—. In certain embodiments, Cy^{D} is an optionally substituted heterocyclyl (e.g., optionally substituted oxetanyl, optionally substituted azetidinyl, optionally substituted piperidinyl). In certain embodiments, Cy^{D} is an optionally substituted carbocyclyl (e.g., optionally substituted cyclopentyl). In certain embodiments, Cy^{D} is an optionally substituted heteroaryl (e.g., optionally substituted benzoimidazolyl). In certain embodiments, L_{D} is a linker group L_{B}, e.g., L_{D} is —C(O)N(R)—. In certain embodiments, L_{D} is —O—. In certain embodiments, [symbol] represents a single bond.

In certain embodiments of formula (A-V^{D}-ii), wherein R¹² is hydrogen and R¹³ is —OR¹, provided is a compound of formula (V^{D}-ii-a):

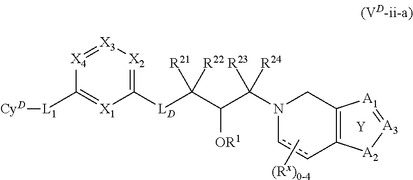

or a pharmaceutically acceptable salt thereof.

In certain embodiments, of formula (V^{D}-ii-a), wherein R²¹-R²⁴ is hydrogen, the heterocyclic ring fused to Ring Y does not comprise any R^{x} substituents, and [symbol] represents a single bond, provided is a compound of formula (V^{D}-ii-b) or (V^{D}-ii-c):

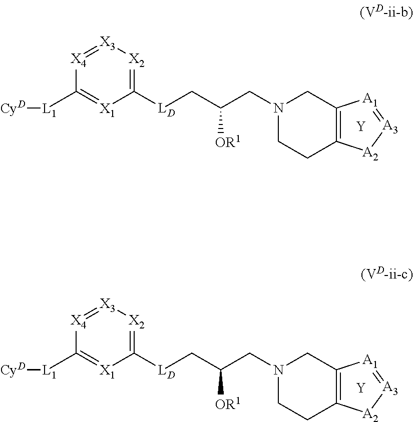

or a pharmaceutically acceptable salt thereof.

In certain embodiments of formula (A-V^{D}), wherein the right hand ring system of formula (x) is a bicyclic ring system of formula (iii), provided is a compound of formula (A-V^{D}-iii):

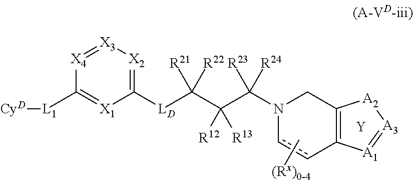

or a pharmaceutically acceptable salt thereof. In certain embodiments, Ring Y does not comprise any R^{x} substituents. In certain embodiments, the heterocyclic ring fused to Ring Y does not comprise any R^{x} substituents, i.e., —(R^{x})₀₋₄ is —(R^{x})₀ and may be depicted absent. In certain embodiments, A₂ is S. In certain embodiments, A₂ is NH or NR^{x}. In certain embodiments, A₁ is CH. In certain embodiments, A₁ is N. In certain embodiments, A₃ is CH or CR^{x}. In certain embodiments, A₂ is S, and A₁ and A₃ are CH or CR^{x}. In certain embodiments, A₂ is NH or NR^{x}, and A₁ and A₃ are CH or CR^{x}. In certain embodiments, A₂ is NH or NR^{x}, A₁ is N, and A₃ is CH or CR^{x}. In certain embodiments, R^{x} is optionally substituted alkyl (e.g., —CH₃ or —CF₃) or optionally substituted aryl (e.g., optionally substituted phenyl). In certain embodiments, R^{x} attached to a nitrogen atom on Ring Y is optionally substituted alkyl, e.g., —CH₃. In certain embodiments, each of R²¹, R²², R²³, and R²⁴ is hydrogen. In certain embodiments, R¹ is hydrogen. In some embodiments, carbon attached to —OR¹ has (S)-stereochemistry. In some embodiments, carbon attached to —OR¹ has (R)-stereochemistry. In certain embodiments, X₁ is CH. In certain embodiments, X₂ is N. In certain embodiments, X₃ is CH. In certain embodiments, X₄ is N. In certain embodiments, X₂ and X₄ is N. In certain embodiments, X₁ and X₃ are CH. In certain embodiments, X₂ and X₄ is N and X₁ and X₃ are CH. In certain embodiments, each of X₁, X₂, X₃, and X₄ is CH. In certain embodiments, L₁ is a bond. In certain embodiments, L₁ is NH. In certain embodiments, L₁ is —N(H)CH₂—. In certain embodiments, Cy^{D} is an optionally substituted heterocyclyl (e.g., optionally substituted oxetanyl, optionally substituted azetidinyl, optionally substituted piperidinyl). In certain embodiments, Cy^{D} is an optionally substituted carbocyclyl (e.g., optionally substituted cyclopentyl). In certain embodiments, Cy^{D} is an optionally substituted heteroaryl (e.g., optionally substituted benzoimidazolyl). In certain embodiments, L_{D} is a linker group L_{B}, e.g., L_{D} is —C(O)N(R)—. In certain embodiments, L_{D} is —O—. In certain embodiments, [symbol] represents a single bond.

In certain embodiments of formula (A-V^{D}-iii), wherein R¹² is hydrogen and R¹³ is —OR¹, provided is a compound of formula (V^{D}-iii-a):

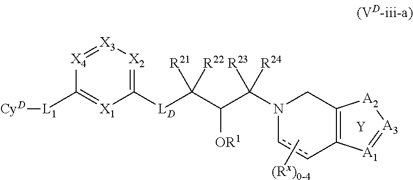

or a pharmaceutically acceptable salt thereof.

In certain embodiments, of formula (V^{D}-iii-a), wherein R²¹-R²⁴ is hydrogen, the heterocyclic ring fused to Ring Y does not comprise any R^{x} substituents, and [symbol] represents a single bond, provided is a compound of formula (V^{D}-iii-b) or (V^{D}-iii-c):

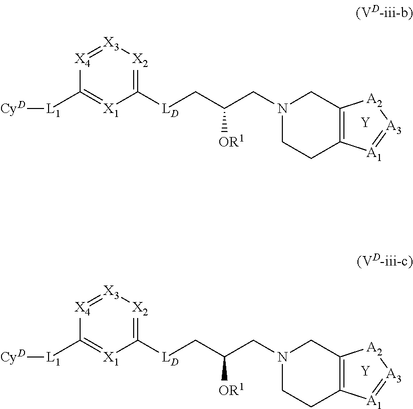

or a pharmaceutically acceptable salt thereof.

In certain embodiments of formula (V^{D}), wherein the right hand ring system of formula (x) is a bicyclic ring system of formula (iv), provided is a compound of formula (V^{D}-iv):

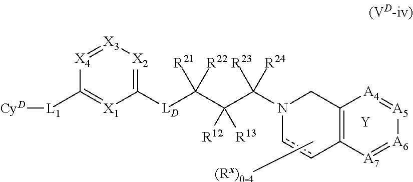

or a pharmaceutically acceptable salt thereof. In certain embodiments, Ring Y does not comprise any R^{x} substituents. In certain embodiments, the heterocyclic ring fused to Ring Y does not comprise any R^{x} substituents, i.e., —(R^{x})₀₋₄ is —(R^{x})₀ and may be depicted absent. In certain embodiments, A₇ is N and A₄, A₅, and A₆ are CH. In certain embodiments, A₆ is N and A₄, A₅, and A₇ are CH. In certain embodiments, A₅ is N and A₄, A₆, and A₇ are CH. In certain embodiments, A₄ is N and A₅, A₆, and A₇ are CH. In certain embodiments, each of R²¹, R²², R²³, and R²⁴ is hydrogen. In certain embodiments, R¹ is hydrogen. In some embodiments, carbon attached to —OR¹ has (S)-stereochemistry. In some embodiments, carbon attached to —OR¹ has (R)-stereochemistry. In certain embodiments, X₁ is CH. In certain embodiments, X₂ is N. In certain embodiments, X₃ is CH. In certain embodiments, X₄ is N. In certain embodiments, X₂ and X₄ is N. In certain embodiments, X₁ and X₃ are CH. In certain embodiments, X₂ and X₄ is N and X₁ and X₃ are CH. In certain embodiments, each of X₁, X₂, X₃, and X₄ is CH. In certain embodiments, L₁ is a bond. In certain embodiments, L₁ is NH. In certain embodiments, L₁ is —N(H)CH₂—. In certain embodiments, Cy^{D} is an optionally substituted heterocyclyl (e.g., optionally substituted oxetanyl, optionally substituted azetidinyl, optionally substituted piperidinyl). In certain embodiments, Cy^{D} is an optionally substituted carbocyclyl (e.g., optionally substituted cyclopentyl). In certain embodiments, Cy^{D} is an optionally substituted heteroaryl (e.g., optionally substituted benzoimidazolyl). In certain embodiments, L_{D} is a linker group L_{B}, e.g., L_{D} is —C(O)N(R)—. In certain embodiments, L_{D} is —O—. In certain embodiments, [symbol] represents a single bond.

In certain embodiments of formula (A-V^{D}-iv), wherein R¹² is hydrogen and R¹³ is —OR¹, provided is a compound of formula (V^{D}-iv-a):

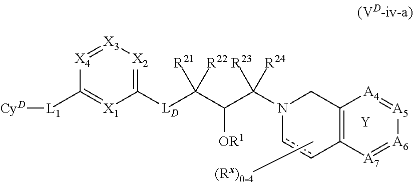

or a pharmaceutically acceptable salt thereof.

In certain embodiments, of formula (V^{D}-iv-a), wherein R²¹-R²⁴ is hydrogen, the heterocyclic ring fused to Ring Y does not comprise any R^{x} substituents, and [symbol] represents a single bond, provided is a compound of formula (V^{D}-iv-b) or (V^{D}-iv-b′):

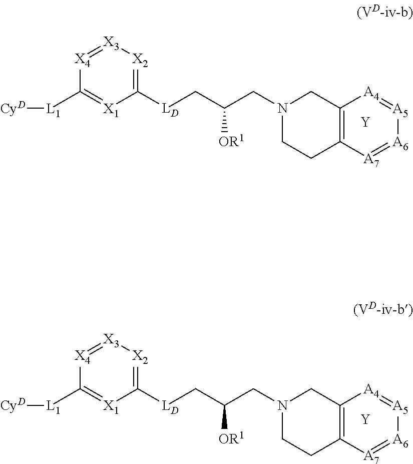

or a pharmaceutically acceptable salt thereof.

In certain embodiments of formula (V^{D}), wherein the right hand ring system is a bicyclic ring system of formula (x-1), provided is a compound of formula (V^{D}-x-1):

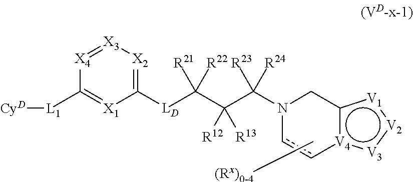

or a pharmaceutically acceptable salt thereof. In certain embodiments, V₄ is N. In certain embodiments, V₁ is N. In certain embodiments, V₂ is N. In certain embodiments, V₃ is CH or CR^{x}. In certain embodiments, V₁, V₂, and V₄ are N, and V₃ is CH or CR^{x}. In certain embodiments, R^{x} is optionally substituted alkyl (e.g., —CH₃ or —CF₃). In certain embodiments, each of R²¹, R²², R²³, and R²⁴ is hydrogen. In certain embodiments, R¹ is hydrogen. In some embodiments, carbon attached to —OR¹ has (S)-stereochemistry. In some embodiments, carbon attached to —OR¹ has (R)-stereochemistry. In certain embodiments, X₁ is CH. In certain embodiments, X₂ is N. In certain embodiments, X₃ is CH. In certain embodiments, X₄ is N. In certain embodiments, X₂ and X₄ is N. In certain embodiments, X₁ and X₃ are CH. In certain embodiments, X₂ and X₄ is N and X₁ and X₃ are CH. In certain embodiments, each of X₁, X₂, X₃, and X₄ is CH. In certain embodiments, L₁ is a bond. In certain embodiments, L₁ is NH. In certain embodiments, L₁ is —N(H)CH₂—. In certain embodiments, Cy^{D} is an optionally substituted heterocyclyl (e.g., optionally substituted oxetanyl, optionally substituted azetidinyl, optionally substituted piperidinyl). In certain embodiments, Cy^{D} is an optionally substituted carbocyclyl (e.g., optionally substituted cyclopentyl). In certain embodiments, Cy^{D} is an optionally substituted heteroaryl (e.g., optionally substituted benzoimidazolyl). In certain embodiments, L_{D} is a linker group L_{B}, e.g., L_{D} is —C(O)N(R)—. In certain embodiments, L_{D} is —O—. In certain embodiments, [symbol] represents a single bond.

In certain embodiments of formula (V^{D}-x-1), wherein R¹² is hydrogen and R¹³ is —OR¹, provided is a compound of formula (V^{D}-x-1-a):

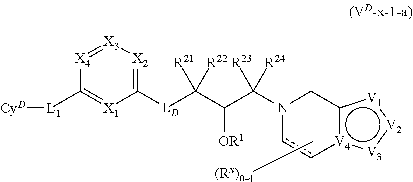

or a pharmaceutically acceptable salt thereof.

In certain embodiments, of formula (V^{D}-x-1-a), wherein R²¹-R²⁴ is hydrogen, the heterocyclic ring fused to Ring Y does not comprise any R^{x} substituents, and [symbol] represents a single bond, provided is a compound of formula (V^{D}-x-1-b) or (V^{D}-x-1-b′).

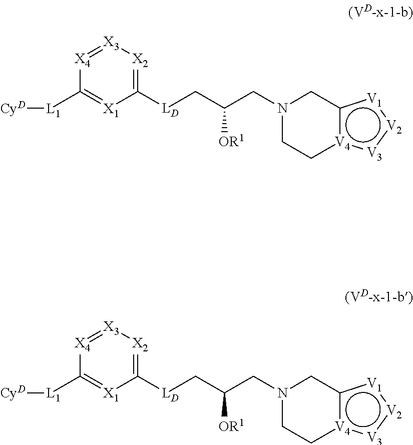

or a pharmaceutically acceptable salt thereof.

In certain embodiments of formula (V^{D}), wherein the right hand ring system is a bicyclic ring system of formula (y-d), provided is a compound of formula (V^{D}-y-d):

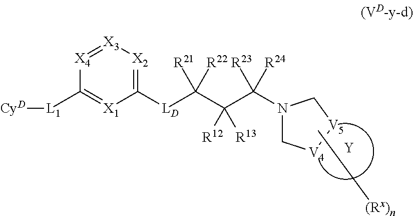

or a pharmaceutically acceptable salt thereof. In certain embodiments, the right hand ring system is a ring system of formula:

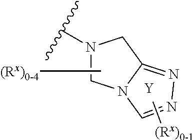

In certain embodiments, R^{x} is optionally substituted alkyl (e.g., —CH₃ or —CF₃). In certain embodiments, each of R²¹, R²², R²³, and R²⁴ is hydrogen. In certain embodiments, R¹ is hydrogen. In some embodiments, carbon attached to —OR¹ has (S)-stereochemistry. In some embodiments, carbon attached to —OR¹ has (R)-stereochemistry. In certain embodiments, X₁ is CH. In certain embodiments, X₂ is N. In certain embodiments, X₃ is CH. In certain embodiments, X₄ is N. In certain embodiments, X₂ and X₄ is N. In certain embodiments, X₁ and X₃ are CH. In certain embodiments, X₂ and X₄ is N and X₁ and X₃ are CH. In certain embodiments, each of X₁, X₂, X₃, and X₄ is CH. In certain embodiments, L₁ is a bond. In certain embodiments, L₁ is NH. In certain embodiments, L₁ is —N(H)CH₂—. In certain embodiments, Cy^{D} is an optionally substituted heterocyclyl (e.g., optionally substituted oxetanyl, optionally substituted azetidinyl, optionally substituted piperidinyl). In certain embodiments, Cy^{D} is an optionally substituted carbocyclyl (e.g., optionally substituted cyclopentyl). In certain embodiments, Cy^{D} is an optionally substituted heteroaryl (e.g., optionally substituted benzoimidazolyl). In certain embodiments, L_{D} is a linker group L_{B}, e.g., L_{D} is —C(O)N(R)—. In certain embodiments, L_{D} is —O—. In certain embodiments, [symbol] represents a single bond.

In certain embodiments of formula (V^{D}-y-d), wherein R¹² is hydrogen and R¹³ is —OR¹, provided is a compound of formula (V^{D}-y-d-a):

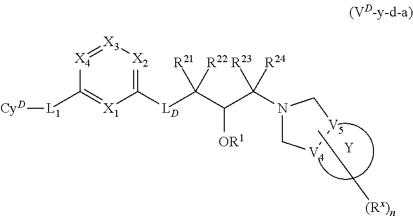

or a pharmaceutically acceptable salt thereof.

In certain embodiments, of formula (V^{D}-x-1-a), wherein R²¹-R²⁴ is hydrogen, provided is a compound of formula (V^{D}-y-d-b) or (V^{D}-y-d-b′):

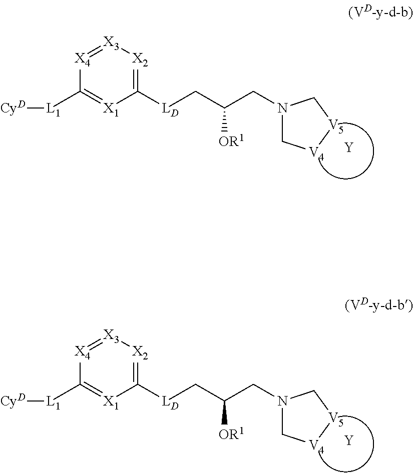

or a pharmaceutically acceptable salt thereof.

In certain embodiments of formula (A-I^{A}), wherein the right hand ring system of formula (x) is a bicyclic ring system of formula (i), provided is a compound of formula (A-I^{A}-i)

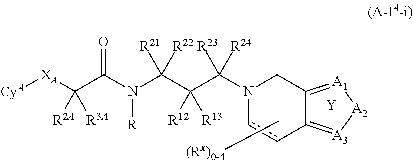

or a pharmaceutically acceptable salt thereof. In certain embodiments, Ring Y does not comprise any R^{x} substituents. In certain embodiments, the heterocyclic ring fused to Ring Y does not comprise any R^{x} substituents, i.e., —(R^{x})₀₋₄ is —(R^{x})₀ and may be depicted absent. In certain embodiments, A₃ is N and A₁ is CH or CR^{x}. In certain embodiments, A₁ is N and A₃ is CH or CR^{x}. In certain embodiments, A₂ is NH or NR^{x}. In certain embodiments, A₃ is N, A₁ is CH or CR^{x}, and A₂ is NH or NR^{x}. In certain embodiments, A₁ is N, A₃ is CH or CR^{x}, and A₂ is NH or NR^{x}. In certain embodiments, R^{x} is optionally substituted alkyl (e.g., —CH₃ or —CF₃) or optionally substituted aryl (e.g., optionally substituted phenyl). In certain embodiments, R^{x} attached to a nitrogen atom on Ring Y is optionally substituted alkyl, e.g., —CH₃. In certain embodiments, each of R²¹, R²², R²³, and R²⁴ is hydrogen. In certain embodiments, R¹ is hydrogen. In some embodiments, carbon attached to —OR¹ has (S)-stereochemistry. In some embodiments, carbon attached to —OR¹ has (R)-stereochemistry. In certain embodiments, R is hydrogen. In certain embodiments, X^{A} is O. In certain embodiments, R^{2A} and R^{3A} are hydrogen. In certain embodiments, Cy^{A} is a bicyclic, aromatic ring having 1 nitrogen heteroatom (e.g., optionally substituted quinolone). In certain embodiments, [symbol] represents a single bond.

In certain embodiments of formula (A-I^{A}-i), wherein R¹² is hydrogen and R¹³ is —OR¹, provided is a compound of formula (I^{A}-i-a):

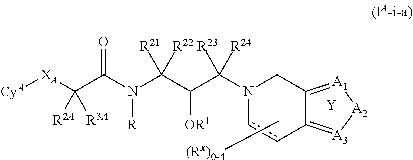

or a pharmaceutically acceptable salt thereof.

In certain embodiments, of formula (I^{A}-i-a), wherein R²¹-R²⁴ is hydrogen, the heterocyclic ring fused to Ring Y does not comprise any R^{x} substituents, and [symbol] represents a single bond, provided is a compound of formula (I^{A}-i-b) or (I^{A}-i-c)

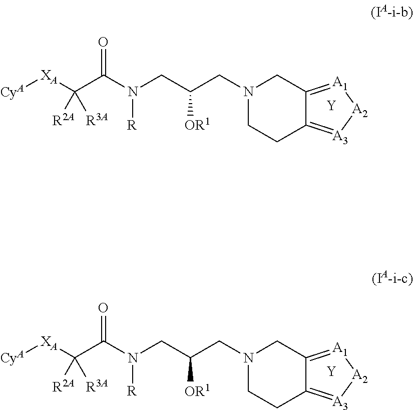

or a pharmaceutically acceptable salt thereof.

In certain embodiments of formula (A-I^{A}), wherein the right hand ring system of formula (x) is a bicyclic ring system of formula (ii), provided is a compound of formula (A-I^{A}-ii):

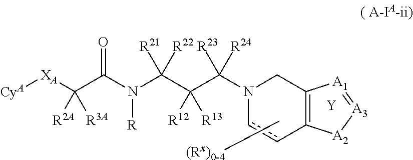

or a pharmaceutically acceptable salt thereof. In certain embodiments, Ring Y does not comprise any R^{x} substituents. In certain embodiments, the heterocyclic ring fused to Ring Y does not comprise any R^{x} substituents, i.e., —(R^{x})₀₋₄ is —(R^{x})₀ and may be depicted absent. In certain embodiments, A₂ is S. In certain embodiments, A₂ is NH or NR^{x}. In certain embodiments, A₁ is CH or CR^{x}. In certain embodiments, A₁ is N. In certain embodiments A₂ is S, and A₁ and A₃ are CH or CR^{x}. In certain embodiments A₂ is S, and A₁ and A₃ are CH. In certain embodiments, A₁ is N and A₂ is NH or NR^{x}. In certain embodiments, A₁ is CH or CR^{x} and A₂ is NH or NR^{x}. In certain embodiments, A₃ is CH or CR^{x}. In certain embodiments, A₃ is N. In certain embodiments, A₃ is N and A₂ is NH or NR^{x}. In certain embodiments, A₃ is CH or CR^{x} and A₂ is NH or NR^{x}. In certain embodiments A₂ is NH or NR^{x}, and A₁ and A₃ are CH or CR^{x}. In certain embodiments A₂ is NH or NR^{x}, A₁ is CR^{x}, and A₃ is CH. In certain embodiments A₂ is NH or NR^{x}, A₁ is CH, and A₃ is CR^{x}. In certain embodiments A₂ is NH or NR^{x}, A₁ is N, and A₃ is CH or CR^{x}. In certain embodiments A₂ is NH or NR^{x}, A₁ is N, and A₃ is CH. In certain embodiments A₂ is NH or NR^{x}, A₁ is N, and A₃ is CR^{x}. In certain embodiments A₂ is NH or NR^{x}, A₃ is N, and A₁ is CH or CR^{x}. In certain embodiments A₂ is NH or NR^{x}, A₃ is N, and A₁ is CH. In certain embodiments A₂ is NH or NR^{x}, A₃ is N, and A₁ is CR^{x}. In certain embodiments, R^{x} is optionally substituted alkyl (e.g., —CH₃ or —CF₃) or optionally substituted aryl (e.g., optionally substituted phenyl). In certain embodiments, R^{x} attached to a nitrogen atom on Ring Y is optionally substituted alkyl, e.g., —CH₃. In certain embodiments, each of R²¹, R²², R²³, and R²⁴ is hydrogen. In certain embodiments, R¹ is hydrogen. In some embodiments, carbon attached to —OR¹ has (S)-stereochemistry. In some embodiments, carbon attached to —OR¹ has (R)-stereochemistry. In certain embodiments, R is hydrogen. In certain embodiments, X^{A} is O. In certain embodiments, R^{2A} and R^{3A} are hydrogen. In certain embodiments, Cy^{A} is a bicyclic, aromatic ring having 1 nitrogen heteroatom (e.g., optionally substituted quinolone). In certain embodiments, [symbol] represents a single bond.

In certain embodiments of formula (A-I^{A}-ii), wherein R¹² is hydrogen and R¹³ is —OR¹, provided is a compound of formula (I^{A}-ii-a):

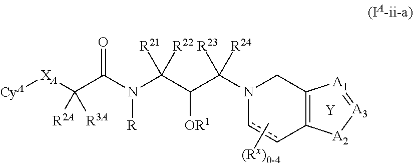

or a pharmaceutically acceptable salt thereof.

In certain embodiments, of formula (I^{A}-ii-a), wherein R²¹-R²⁴ is hydrogen, the heterocyclic ring fused to Ring Y does not comprise any R^{x} substituents, and [symbol] represents a single bond, provided is a compound of formula (I^{A}-ii-b) or (I^{A}-ii-c):

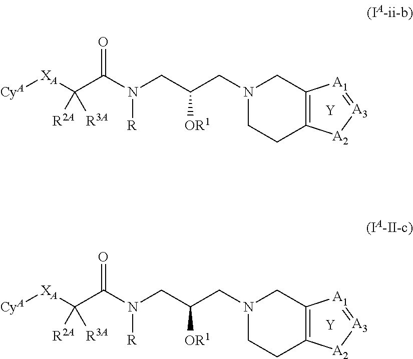

or a pharmaceutically acceptable salt thereof.

In certain embodiments of formula (A-I^{A}), wherein the right hand ring system of formula (x) is a bicyclic ring system of formula (iii), provided is a compound of formula (A-I^{A}-iii):

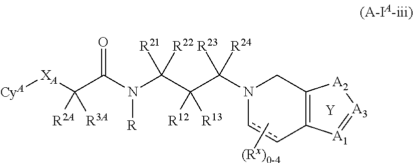

or a pharmaceutically acceptable salt thereof. In certain embodiments, Ring Y does not comprise any R^{x} substituents. In certain embodiments, the heterocyclic ring fused to Ring Y does not comprise any R^{x} substituents, i.e., —(R^{x})₀₋₄ is —(R^{x})₀ and may be depicted absent. In certain embodiments, A₂ is S. In certain embodiments, A₂ is NH or NR^{x}. In certain embodiments, A₁ is CH. In certain embodiments, A₁ is N. In certain embodiments, A₃ is CH or CR^{x}. In certain embodiments, A₂ is S, and A₁ and A₃ are CH or CR^{x}. In certain embodiments, A₂ is NH or NR^{x}, and A₁ and A₃ are CH or CR^{x}. In certain embodiments, A₂ is NH or NR^{x}, A₁ is N, and A₃ is CH or CR^{x}. In certain embodiments, R^{x} is optionally substituted alkyl (e.g., —CH₃ or —CF₃) or optionally substituted aryl (e.g., optionally substituted phenyl). In certain embodiments, R^{x} attached to a nitrogen atom on Ring Y is optionally substituted alkyl, e.g., —CH₃. In certain embodiments, each of R²¹, R²², R²³, and R²⁴ is hydrogen. In certain embodiments, R¹ is hydrogen. In some embodiments, carbon attached to —OR¹ has (S)-stereochemistry. In some embodiments, carbon attached to —OR¹ has (R)-stereochemistry. In certain embodiments, R is hydrogen. In certain embodiments, X^{A} is O. In certain embodiments, R^{2A} and R^{3A} are hydrogen. In certain embodiments, Cy^{A} is a bicyclic, aromatic ring having 1 nitrogen heteroatom (e.g., optionally substituted quinolone). In certain embodiments, [symbol] represents a single bond.

In certain embodiments of formula (A-I^{A}-iii), wherein R¹² is hydrogen and R¹³ is —OR¹, provided is a compound of formula (I^{A}-iii-a):

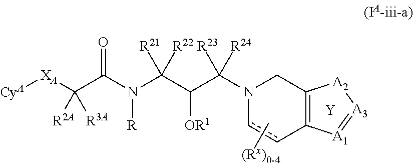

or a pharmaceutically acceptable salt thereof.

In certain embodiments, of formula (I^{A}-iii-a), wherein R²¹-R²⁴ is hydrogen, the heterocyclic ring fused to Ring Y does not comprise any R^{x} substituents, and [symbol] represents a single bond, provided is a compound of formula (I^{A}-iii-b) or (I^{A}-iii-c):

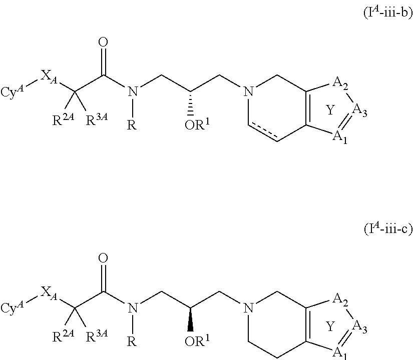

or a pharmaceutically acceptable salt thereof.

In certain embodiments of formula (A-I^{A}), wherein the right hand ring system of formula (x) is a bicyclic ring system of formula (iv), provided is a compound of formula (A-I^{A}-iv):

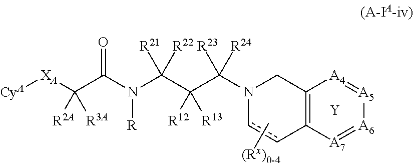

or a pharmaceutically acceptable salt thereof. In certain embodiments, Ring Y does not comprise any R^{x} substituents. In certain embodiments, the heterocyclic ring fused to Ring Y does not comprise any R^{x} substituents, i.e., —(R^{x})₀₋₄ is —(R^{x})₀ and may be depicted absent. In certain embodiments, A₇ is N and A₄, A₅, and A₆ are CH. In certain embodiments, A₆ is N and A₄, A₅, and A₇ are CH. In certain embodiments, A₅ is N and A₄, A₆, and A₇ are CH. In certain embodiments, A₄ is N and A₅, A₆, and A₇ are CH. In certain embodiments, each of R²¹, R²², R²³, and R²⁴ is hydrogen. In certain embodiments, R¹ is hydrogen. In some embodiments, carbon attached to —OR¹ has (S)-stereochemistry. In some embodiments, carbon attached to —OR¹ has (R)-stereochemistry. In certain embodiments, R is hydrogen. In certain embodiments, X^{A} is O. In certain embodiments, R^{2A} and R^{3A} are hydrogen. In certain embodiments, Cy^{A} is a bicyclic, aromatic ring having 1 nitrogen heteroatom (e.g., optionally substituted quinolone). In certain embodiments, [symbol] represents a single bond.

In certain embodiments of Formula (A-I^{A}-iv), wherein R¹² is hydrogen and R¹³ is —OR¹, provided is a compound of Formula (I^{A}-iv-a):

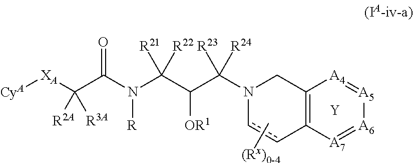

or a pharmaceutically acceptable salt thereof.

In certain embodiments, of Formula (I^{A}-iv-a), wherein R²¹-R²⁴ is hydrogen, the heterocyclic ring fused to Ring Y does not comprise any R^{x} substituents, and [symbol] represents a single bond, provided is a compound of formula (I^{A}-iv-b) or (I^{A}-iv-c):

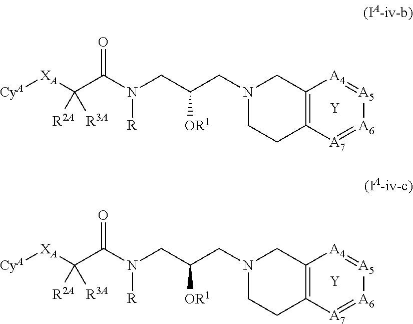

or a pharmaceutically acceptable salt thereof.

In certain embodiments of Formula (A-I^{A}), wherein the right hand ring system is a bicyclic ring system of formula (x-1), provided is a compound of formula (A-I^{A}-x-1):

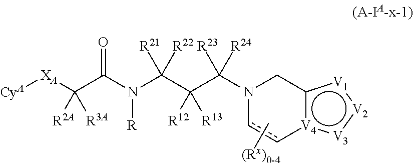

or a pharmaceutically acceptable salt thereof. In certain embodiments, V₄ is N. In certain embodiments, V₁ is N. In certain embodiments, V₂ is N. In certain embodiments, V₃ is CH or CR^{x}. In certain embodiments, V₁, V₂, and V₄ are N, and V₃ is CH or CR^{x}. In certain embodiments, R^{x} is optionally substituted alkyl (e.g., —CH₃ or —CF₃). In certain embodiments, each of R²¹, R²², R²³, and R²⁴ is hydrogen. In certain embodiments, R¹ is hydrogen. In some embodiments, carbon attached to —OR¹ has (S)-stereochemistry. In some embodiments, carbon attached to —OR¹ has (R)-stereochemistry. In certain embodiments, R is hydrogen. In certain embodiments, X^{A} is O. In certain embodiments, R^{2A} and R^{3A} are hydrogen. In certain embodiments, Cy^{A} is a bicyclic, aromatic ring having 1 nitrogen heteroatom (e.g., optionally [symbol] substituted quinolone). In certain embodiments, [symbol] represents a single bond.

In certain embodiments of Formula (A-I^{A}-x-1), wherein R¹² is hydrogen and R¹³ is —OR¹, provided is a compound of formula (I^{A}-x-1-a):

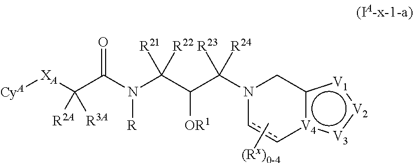

or a pharmaceutically acceptable salt thereof.

In certain embodiments, of Formula (I^{A}-x-1-a), wherein R²¹-R²⁴ is hydrogen, the heterocyclic ring fused to Ring Y does not comprise any R^{x} substituents, and [symbol] represents a single bond, provided is a compound of formula (I^{A}-x-1-b) or (I^{A}-x-1-c):

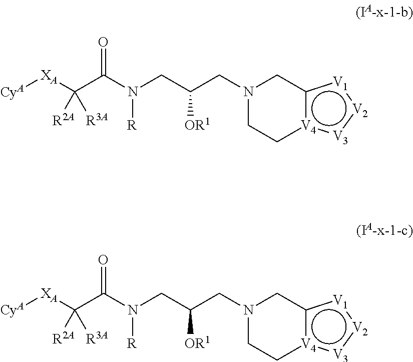

or a pharmaceutically acceptable salt thereof.

In certain embodiments of Formula (A-I^{A}), wherein the right hand ring system is a bicyclic ring system of formula (y-d), provided is a compound of formula (A-I^{A}-y-d-1):

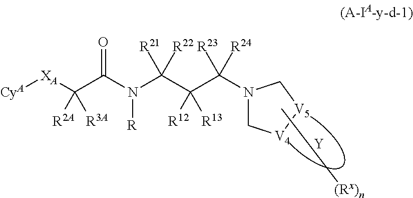

or a pharmaceutically acceptable salt thereof. In certain embodiments, the right hand ring system is a ring system of formula:

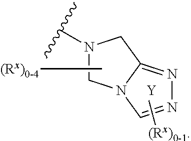

In certain embodiments, R^{x} is optionally substituted alkyl (e.g., —CH₃ or —CF₃). In certain embodiments, each of R²¹, R²², R²³, and R²⁴ is hydrogen. In certain embodiments, R¹ is hydrogen. In some embodiments, carbon attached to —OR¹ has (S)-stereochemistry. In some embodiments, carbon attached to —OR¹ has (R)-stereochemistry. In certain embodiments, R is hydrogen. In certain embodiments, X^{A} is O. In certain embodiments, R^{2A} and R^{3A} are hydrogen. In certain embodiments, Cy^{A} is a bicyclic, aromatic ring having 1 nitrogen heteroatom (e.g., optionally substituted quinolone). In certain embodiments, [symbol] represents a single bond.

In certain embodiments of Formula (A-I^{A}-y-d), wherein R¹² is hydrogen and R¹³ is —OR¹, provided is a compound of formula (I^{A}-y-d-a):

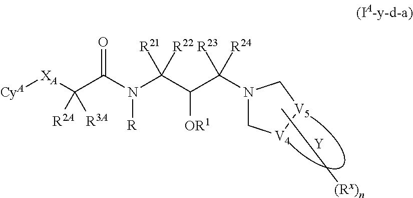

or a pharmaceutically acceptable salt thereof.

In certain embodiments, of Formula (I^{A}-y-d-a), wherein R²¹-R²⁴ is hydrogen, provided is a compound of formula (I^{A}-y-d-b) or (I^{A}-y-d-c):

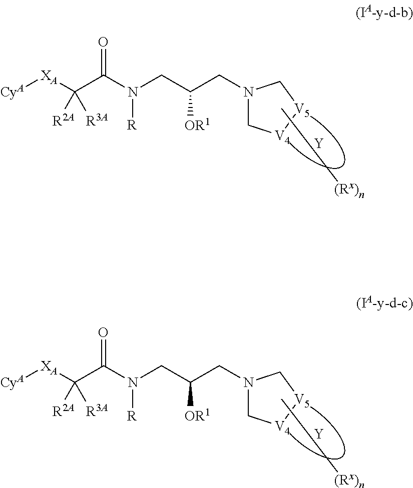

or a pharmaceutically acceptable salt thereof.

In certain embodiments of Formula (A-I^{B}), a provided compound is of Formula:

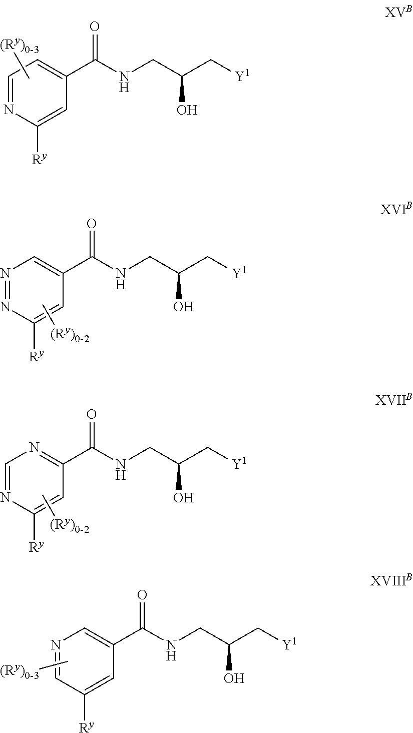

or a pharmaceutically acceptable salt thereof, wherein each Y¹ and R^{y} for Formula (XV^{B}), (XVI^{B}), (XVII^{B}), or (XVIII^{B}) is independently as described herein.

In some embodiments of Formula (XV^{B}), (XVI^{B}), (XVII^{B}), or (XVIII^{B}), when the nitrogen-containing heteroaryl moiety has only one substituent R^{y}, R^{y} is not halo (e.g., F or Cl) or optionally substituted alkyl. In some embodiments of Formula (XV^{B}), (XVI^{B}), (XVII^{B}), or (XVIII^{B}), when the nitrogen-containing heteroaryl moiety has only one substituent R^{y}, R^{y} is not halo (e.g., F or Cl) or C₁₋₃ alkyl (e.g. methyl, ethyl, n-propyl, or iso-propyl). In some embodiments of Formula (XV^{B}), (XVI^{B}), (XVII^{B}), or (XVIII^{B}), when the nitrogen-containing heteroaryl has only one substituent R^{y}, R^{y} is —N(R^{B})₂, wherein R^{B} is as generally defined herein. In some embodiments of Formula (XV^{B}), (XVI^{B}), (XVII^{B}), or (XVIII^{B}), when the nitrogen-containing heteroaryl has only one substituent R^{y}, R^{y} is —N(R^{B})₂, and at least one R^{B} is optionally substituted heterocyclyl. In some embodiments of Formula (XV^{B}), (XVI^{B}), (XVII^{B}), or (XVIII^{B}), when the nitrogen-containing heteroaryl has only one substituent R^{y}, R^{y} is —NHR^{B}, wherein R^{B} is as generally defined herein. In some embodiments of Formula (XV^{B}), (XVI^{B}), (XVII^{B}), or (XVIII^{B}), when the nitrogen-containing heteroaryl has only one substituent R^{y}, R^{y} is —NHR^{B}, wherein R^{B} is optionally substituted heterocyclyl.

In certain embodiments, a provided compound is of Formula (XV^{B}-a), (XVI^{B}-a), (XVII^{B}-a), or (XVIII^{B}-a):

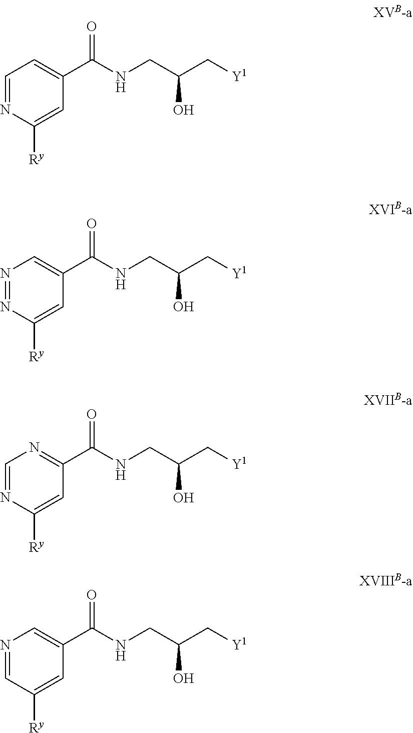

or a pharmaceutically acceptable salt thereof, wherein R^{y} for Formula (XV^{B}-a), (XVI^{B}-a), (XVII^{B}-a), or (XVIII^{B}-a) is as generally described herein. In some embodiments, e.g. for Formula (XV^{B}-a), (XVI^{B}-a), (XVII^{B}-a), or (XVIII^{B}-a), R^{y} is —OR^{A}, wherein R^{A} is optionally substituted alkyl, optionally substituted carbocyclyl, optionally substituted heterocyclyl, optionally substituted aryl, or optionally substituted heteroaryl. In some embodiments, e.g. for Formula (XV^{B}-a), (XVI^{B}-a), (XVII^{B}-a), or (XVIII^{B}-a), R^{y} is —OR^{A}, wherein R^{A} is -(optionally substituted alkyl)-(optionally substituted carbocyclyl), -(optionally substituted alkyl)-(optionally substituted heterocyclyl), or -(optionally substituted alkyl)-(optionally substituted heteroaryl). In some embodiments, e.g. for Formula (XV^{B}-a), (XVI^{B}-a), (XVII^{B}-a), or (XVIII^{B}-a), R^{y} is —OR^{A}, wherein R^{A} is optionally substituted heterocyclyl. In some embodiments, e.g. for Formula (XV^{B}-a), (XVI^{B}-a), (XVII^{B}-a), or (XVIII^{B}-a), R^{y} is —OR^{A}, wherein R^{A} is optionally substituted heteroaryl. In some embodiments, e.g. for Formula (XV^{B}-a), (XVI^{B}-a), (XVII^{B}-a), or (XVIII^{B}-a), R^{y} is —OR^{A}, wherein R^{A} is optionally substituted carbocyclyl. In some embodiments, e.g. for Formula (XV^{B}-a), (XVI^{B}-a), (XVII^{B}-a), or (XVIII^{B}-a), R^{y} is —N(R^{B})₂, wherein R^{B} is hydrogen, optionally substituted alkyl, optionally substituted carbocyclyl, optionally substituted heterocyclyl, optionally substituted aryl, or optionally substituted heteroaryl. In some embodiments, e.g. for Formula (XV^{B}-a), (XVI^{B}-a), (XVII^{B}-a), or (XVIII^{B}-a), R^{y} is —NHR^{B}. In some embodiments, e.g. for Formula (XV^{B}-a), (XVI^{B}-a), (XVII^{B}-a), or (XVIII^{B}-a), R^{y} is —NHR^{B}, wherein R^{B} is optionally substituted alkyl, optionally substituted carbocyclyl, optionally substituted heterocyclyl, optionally substituted aryl, or optionally substituted heteroaryl. In some embodiments, e.g. for Formula (XV^{B}-a), (XVI^{B}-a), (XVII^{B}-a), or (XVIII^{B}-a), R^{y} is —NHR^{B}, wherein R^{B} is -(optionally substituted alkyl)-(optionally substituted carbocyclyl)-, -(optionally substituted alkyl)-(optionally substituted heterocyclyl)-, or -(optionally substituted alkyl)-(optionally substituted heteroaryl)-. In some embodiments, e.g. for Formula (XV^{B}-a), (XVI^{B}-a), (XVII^{B}-a), or (XVIII^{B}-a), R^{y} is —NHR^{B}, wherein R^{B} is optionally substituted heterocyclyl. In some embodiments, e.g. for Formula (XV^{B}-a), (XVI^{B}-a), (XVII^{B}-a), or (XVIII^{B}-a), R^{y} is —NHR^{B}, wherein R^{B} is optionally substituted heteroaryl. In some embodiments, e.g. for Formula (XV^{B}-a), (XVI^{B}-a), (XVII^{B}-a), or (XVIII^{B}-a), R^{y} is —NHR^{B}, wherein R^{B} is optionally substituted cycloalkyl. In some embodiments, e.g. for Formula (XV^{B}-a), (XVI^{B}-a), (XVII^{B}-a), or (XVIII^{B}-a), R^{y} is —N(CH₃)R^{B}. In some embodiments, e.g. for Formula (XV^{B}-a), (XVI^{B}-a), (XVII^{B}-a), or (XVIII^{B}-a), R^{y} is —N(CH₃)R^{B}, wherein R^{B} is optionally substituted alkyl, optionally substituted carbocyclyl, optionally substituted heterocyclyl, optionally substituted aryl, or optionally substituted heteroaryl. In some embodiments, e.g. for Formula (XV^{B}-a), (XVI^{B}-a), (XVII^{B}-a), or (XVIII^{B}-a), R^{y} is —N(CH₃)R^{B}, wherein R^{B} is -(optionally substituted alkyl)-(optionally substituted carbocyclyl)-, -(optionally substituted alkyl)-(optionally substituted heterocyclyl)-, or -(optionally substituted alkyl)-(optionally substituted heteroaryl)-. In some embodiments, e.g. for Formula (XV^{B}-a), (XVI^{B}-a), (XVII^{B}-a), or (XVIII^{B}-a), R^{y} is —N(R^{B})₂, wherein one R^{B} is optionally substituted heterocyclyl, and the other R^{B} is C₁₋₄ alkyl. In some embodiments, e.g. for Formula (XV^{B}-a), (XVI^{B}-a), (XVII^{B}-a), or (XVIII^{B}-a), R^{y} is —N(R^{B})₂, wherein one R^{B} is optionally substituted heteroaryl, and the other R^{B} is C₁₋₄ alkyl. In some embodiments, e.g. for Formula (XV^{B}-a), (XVI^{B}-a), (XVII^{B}-a), or (XVIII^{B}-a), R^{y} is —N(R^{B})₂, wherein one R^{B} is optionally substituted cycloalkyl, and the other R^{B} is C₁₋₄ alkyl.

In certain embodiments of Formula (XV^{B}-a), wherein R^{y} is —N(R^{B})₂, provided is a compound of Formula (XV^{B}-a-1):

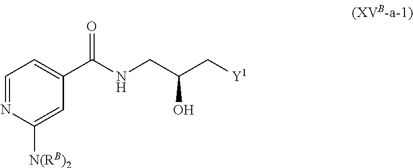

or a pharmaceutically acceptable salt thereof, wherein Y¹ and R^{B} is as generally defined herein. In certain embodiments, at least one R^{B} is an optionally substituted carbocyclic ring or optionally substituted heterocyclic ring, e.g., a 4- to 6-membered optionally substituted carbocyclic ring or a 4- to 6-membered optionally substituted heterocyclic ring.

In certain embodiments of Formula (XV^{B}-a-1), wherein at least one R^{B} is a hydrogen, provided is a compound of Formula (XV^{B}-a-2):

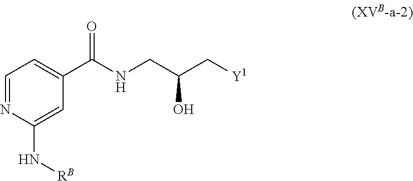

or a pharmaceutically acceptable salt thereof, wherein Y¹ and R^{B} is as generally defined herein. In certain embodiments, R^{B} is an optionally substituted carbocyclic ring or optionally substituted heterocyclic ring. In certain embodiments, R^{B} is an optionally substituted carbocyclic ring, e.g., a 4- to 6-membered optionally substituted carbocyclic ring. In certain embodiments, R^{B} is an optionally substituted heterocyclic ring, e.g., or a 4- to 6-membered optionally substituted heterocyclic ring.

In certain embodiments of Formula (XV^{B}-a-2), wherein R^{B} is an optionally substituted heterocyclic ring, provided is a compound of Formula (XV^{B}-a-3):

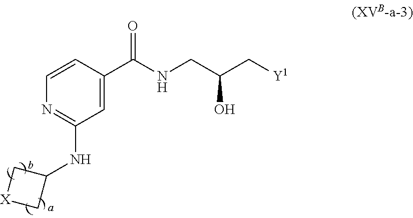

or a pharmaceutically acceptable salt thereof, wherein Y¹ is as generally defined herein, and wherein each instance of a and b is independently 1 or 2, and X is —C(R^{XC})₂—, —O—, —S—, or —NR^{XN}—, wherein each instance of R^{XC} is independently hydrogen, optionally substituted alkyl, optionally substituted carbocyclyl, optionally substituted heterocyclyl, optionally substituted aryl, or optionally substituted heteroaryl; R^{XN} is independently hydrogen, optionally substituted alkyl, optionally substituted carbocyclyl, optionally substituted heterocyclyl, optionally substituted aryl, optionally substituted heteroaryl, —C(═O)R^{XA}, or a nitrogen protecting group; R^{XA} is optionally substituted alkyl, optionally substituted carbocyclyl, optionally substituted heterocyclyl, optionally substituted aryl, or optionally substituted heteroaryl. In certain embodiments, a and b are both 1. In certain embodiments, a and b are both 2. In certain embodiments, X is —O—. In certain embodiments, X is —NR^{XN}—, wherein R^{XN} is as generally defined above. In certain embodiments, X is —NR—N, wherein R^{XN} is optionally substituted alkyl, —C(═O)R^{XA}, or a nitrogen protecting group. In certain embodiments, X is —NR^{XN}—, wherein R^{XN} is —C(═O)R^{A}, wherein R^{XA} is optionally substituted alkyl or optionally substituted carbocyclyl. In certain embodiments, X is —NR^{XN}—, wherein R^{XN} is —C(═O)R^{XA}, wherein R^{XA} is methyl, ethyl, n-propyl, iso-propyl, cyclopropyl, or cyclobutyl. In certain embodiments, a and b are each independently 1 or 2; and X is —O— or —NR^{XN}—, wherein R^{XN} is as generally defined above. In certain embodiments, a and b are each independently 1 or 2; and X is —O— or —NC(═O)R^{XA}, wherein R^{XA} is as generally defined above. In certain embodiments, a and b are both 1; and X is —O— or —NR^{XN}—, wherein R^{XN} is as generally defined above. In certain embodiments, a and b are both 1; and X is —O— or —NC(═O)R^{XA}, wherein R^{XA} is as generally defined above. In certain embodiments, a and b are both 1; and X is —O— or —NC(═O)CH₃. In certain embodiments, a and b are both 1; and X is —O—. In certain embodiments, a and b are both 2; and X is —O— or —NC(═O)CH₃. In certain embodiments, a and b are both 2; and X is —NC(═O)CH₃.

In certain embodiments of Formula (XV^{B}-a-3), wherein a and b are 2, provided is a compound of Formula (XV^{B}-a-4):

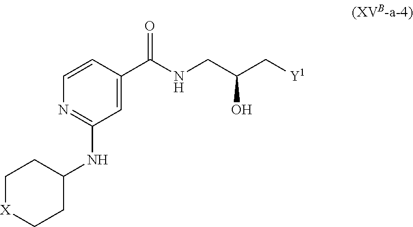

or a pharmaceutically acceptable salt thereof, wherein Y¹ is as generally defined herein, and wherein X is —C(R^{XC})₂—, —O—, —S—, or —NR^{XN}—; each instance of R^{XC} is independently hydrogen, optionally substituted alkyl, optionally substituted carbocyclyl, optionally substituted heterocyclyl, optionally substituted aryl, or optionally substituted heteroaryl; R^{XN} is independently hydrogen, optionally substituted alkyl, optionally substituted carbocyclyl, optionally substituted heterocyclyl, optionally substituted aryl, optionally substituted heteroaryl, —C(═O)R^{XA}, or a nitrogen protecting group; R^{XA} is optionally substituted alkyl, optionally substituted carbocyclyl, optionally substituted heterocyclyl, optionally substituted aryl, or optionally substituted heteroaryl. In certain embodiments, X is —O—. In certain embodiments, X is —NR^{XN}—, wherein R^{XN} is as generally defined above. In certain embodiments, X is —NR^{XN}—, wherein R^{XN} is optionally substituted alkyl, —C(═O)R^{XA}, or a nitrogen protecting group. In certain embodiments, X is —NR^{XN}—, wherein R^{XN} is —C(═O)R^{XA}, wherein R^{XA} is optionally substituted alkyl or optionally substituted carbocyclyl. In certain embodiments, X is —NR^{XN}—, wherein R^{XN} is —C(═O)R^{A}, wherein R^{XA} is methyl, ethyl, n-propyl, iso-propyl, cyclopropyl, or cyclobutyl. In certain embodiments, X is —NC(═O)CH₃.

In certain embodiments of Formula (XV^{B}-a-4), wherein X is —NR^{XN}—, provided is a compound of Formula (XV^{B}-a-5):

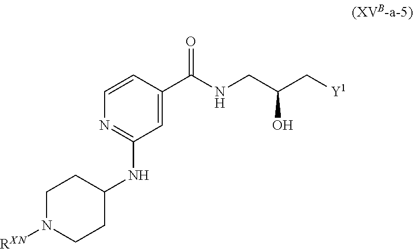

or a pharmaceutically acceptable salt thereof, wherein Y¹ is as generally defined herein, and wherein R^{XN} is independently hydrogen, optionally substituted alkyl, optionally substituted carbocyclyl, optionally substituted heterocyclyl, optionally substituted aryl, optionally substituted heteroaryl, —C(═O)R^{XA}, or a nitrogen protecting group; R^{XA} is optionally substituted alkyl, optionally substituted carbocyclyl, optionally substituted heterocyclyl, optionally substituted aryl, or optionally substituted heteroaryl. In certain embodiments, R^{XN} is optionally substituted alkyl, —C(═O)R^{XA}, or a nitrogen protecting group. In certain embodiments, R^{XN} is —C(═O)R^{XA}, wherein R^{XA} is optionally substituted alkyl or optionally substituted carbocyclyl. In certain embodiments, R^{XN} is —C(═O)R^{XA}, wherein R^{XA} is methyl, ethyl, n-propyl, iso-propyl, cyclopropyl, or cyclobutyl. In certain embodiments, R^{XN} is —C(═O)R^{XA}, wherein R^{XA} is methyl.

In certain embodiments of Formula (XV^{B}-a-5), wherein —NR^{XN}— is —C(═O)R^{XA} provided is a compound of Formula (XV^{B}-a-6):

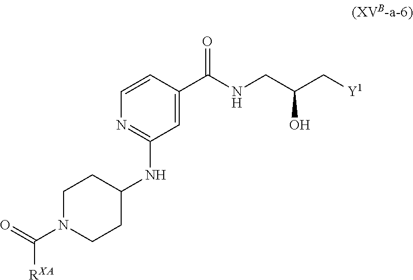

or a pharmaceutically acceptable salt thereof, wherein Y¹ is as generally defined herein, and wherein R^{XA} is optionally substituted alkyl, optionally substituted carbocyclyl, optionally substituted heterocyclyl, optionally substituted aryl, or optionally substituted heteroaryl. In certain embodiments, R^{XA} is optionally substituted alkyl or optionally substituted carbocyclyl. In certain embodiments, R^{XA} is methyl, ethyl, n-propyl, iso-propyl, cyclopropyl, or cyclobutyl. In certain embodiments, R^{XA} is methyl.

In certain embodiments of Formula (XVII^{B}-a), wherein R^{y} is —N(R^{B})₂, provided is a compound of Formula (XVII^{B}-a-1):

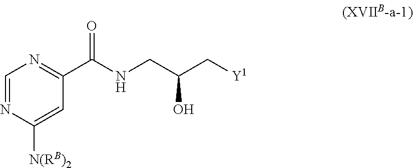

or a pharmaceutically acceptable salt thereof, wherein Y and R^{B} is as generally defined herein. In certain embodiments, at least one R^{B} is an optionally substituted carbocyclic ring or optionally substituted heterocyclic ring, e.g., a 4- to 6-membered optionally substituted carbocyclic ring or a 4- to 6-membered optionally substituted heterocyclic ring.

In certain embodiments of Formula (XVII-a-1), wherein at least one R^{B} is a hydrogen, provided is a compound of Formula (XVII-a-2):

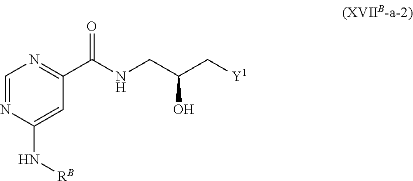

or a pharmaceutically acceptable salt thereof, wherein Y is as generally defined herein, and wherein R^{B} is an optionally substituted carbocyclic ring or optionally substituted heterocyclic ring. In certain embodiments, R^{B} is an optionally substituted carbocyclic ring, e.g., a 4- to 6-membered optionally substituted carbocyclic ring. In certain embodiments, R^{B} is an optionally substituted heterocyclic ring, e.g., or a 4- to 6-membered optionally substituted heterocyclic ring.

In certain embodiments of Formula (XVII^{B}-a-2), wherein R^{B} is an optionally substituted heterocyclic ring, provided is a compound of Formula (XVII^{B}-a-3):

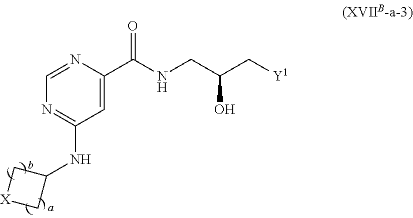

or a pharmaceutically acceptable salt thereof, wherein Y¹ is as generally defined herein, and wherein each instance of a and b is independently 1 or 2, and X is —C(R^{XC})₂—, —O—, —S—, or —NR^{XN}—, wherein each instance of R^{XC} is independently hydrogen, optionally substituted alkyl, optionally substituted carbocyclyl, optionally substituted heterocyclyl, optionally substituted aryl, or optionally substituted heteroaryl; R^{XN} is independently hydrogen, optionally substituted alkyl, optionally substituted carbocyclyl, optionally substituted heterocyclyl, optionally substituted aryl, optionally substituted heteroaryl, —C(═O)R^{XA}, or a nitrogen protecting group; R^{XA} is optionally substituted alkyl, optionally substituted carbocyclyl, optionally substituted heterocyclyl, optionally substituted aryl, or optionally substituted heteroaryl. In certain embodiments, a and b are both 1. In certain embodiments, a and b are both 2. In certain embodiments, X is —O—. In certain embodiments, X is —NR^{XN}—, wherein R^{XN} is as generally defined above. In certain embodiments, X is —NR^{XN}—, wherein R^{XN} is optionally substituted alkyl, —C(═O)R^{XA}, or a nitrogen protecting group. In certain embodiments, X is —NR^{XN}—, wherein R^{XN} is —C(═O)R^{XA}, wherein R^{XA} is optionally substituted alkyl or optionally substituted carbocyclyl. In certain embodiments, X is —NR^{x}—, wherein R^{XN} is —C(═O)R^{XA}, wherein R^{XA} is methyl, ethyl, n-propyl, iso-propyl, cyclopropyl, or cyclobutyl. In certain embodiments, a and b are each independently 1 or 2; and X is —O— or —NR^{XN}—, wherein R^{XN} is as generally defined above. In certain embodiments, a and b are each independently 1 or 2; and X is —O— or —NC(═O)R^{XA}, wherein R^{XA} is as generally defined above. In certain embodiments, a and b are both 1; and X is —O— or —NR^{XN}—, wherein R^{XN} is as generally defined above. In certain embodiments, a and b are both 1; and X is —O— or —NC(═O)R^{XA}, wherein R^{XA} is as generally defined above. In certain embodiments, a and b are both 1; and X is —O— or —NC(═O)CH₃. In certain embodiments, a and b are both 1; and X is —O—. In certain embodiments, a and b are both 2; and X is —O— or —NC(═O)CH₃. In certain embodiments, a and b are both 2; and X is —NC(═O)CH₃.

In certain embodiments of Formula (XVII^{B}-a-3), wherein a and b are 1, provided is a compound of Formula (XVII^{B}-a-4):

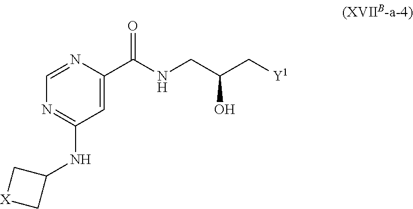

or a pharmaceutically acceptable salt thereof, wherein Y¹ is as generally defined herein, and wherein X is —C(R^{XC})₂—, —O—, —S—, or —NR^{XN}—, wherein each instance of R^{XC} is independently hydrogen, optionally substituted alkyl, optionally substituted carbocyclyl, optionally substituted heterocyclyl, optionally substituted aryl, or optionally substituted heteroaryl; R^{XN} is independently hydrogen, optionally substituted alkyl, optionally substituted carbocyclyl, optionally substituted heterocyclyl, optionally substituted aryl, optionally substituted heteroaryl, —C(═O)R^{XA}, or a nitrogen protecting group; R^{XA} is optionally substituted alkyl, optionally substituted carbocyclyl, optionally substituted heterocyclyl, optionally substituted aryl, or optionally substituted heteroaryl. In certain embodiments, X is —O—. In certain embodiments, X is —NR^{XN}—, wherein R^{XN} is as generally defined above. In certain embodiments, X is —NR^{XN}—, wherein R^{XN} is optionally substituted alkyl, —C(═O)R^{XA}, or a nitrogen protecting group. In certain embodiments, X is —NR^{XN}—, wherein R^{XN} is —C(═O)R^{XA}, wherein R^{XA} is optionally substituted alkyl or optionally substituted carbocyclyl. In certain embodiments, X is —NR^{XN}—, wherein R^{XN} is —C(═O)R^{XA}, wherein R^{XA} is methyl, ethyl, n-propyl, iso-propyl, cyclopropyl, or cyclobutyl. In certain embodiments, X is —NC(═O)CH₃.

In certain embodiments of Formula (XVII^{B}-a-4), wherein X is —NR^{XN}—, provided is a compound of Formula (XVII^{B}-a-5):

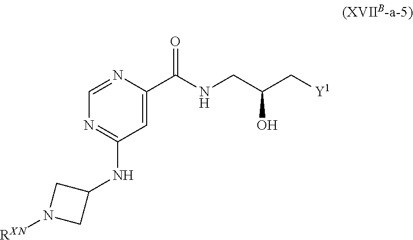

or a pharmaceutically acceptable salt thereof, wherein Y¹ is as generally defined herein, and wherein R^{XN} is independently hydrogen, optionally substituted alkyl, optionally substituted carbocyclyl, optionally substituted heterocyclyl, optionally substituted aryl, optionally substituted heteroaryl, —C(═O)R^{XA}, or a nitrogen protecting group; R^{XA} is optionally substituted alkyl, optionally substituted carbocyclyl, optionally substituted heterocyclyl, optionally substituted aryl, or optionally substituted heteroaryl. In certain embodiments, R^{XN} is optionally substituted alkyl, —C(═O)R^{XA}, or a nitrogen protecting group. In certain embodiments, R^{XN} is —C(═O)R^{XA}, wherein R^{XA} is optionally substituted alkyl or optionally substituted carbocyclyl. In certain embodiments, R^{XN} is —C(═O)R^{XA}, wherein R^{XA} is methyl, ethyl, n-propyl, iso-propyl, cyclopropyl, or cyclobutyl. In certain embodiments, R^{XN} is —C(═O)R^{XA}, wherein R^{XA} is methyl.

In certain embodiments of Formula (XVII^{B}-a-5), wherein —NR^{XN}— is —C(═O)R^{XA} provided is a compound of Formula (XVII^{B}-a-6):

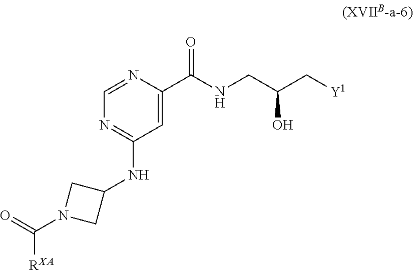

or a pharmaceutically acceptable salt thereof, wherein Y¹ is as generally defined herein, and wherein R^{XA} is optionally substituted alkyl, optionally substituted carbocyclyl, optionally substituted heterocyclyl, optionally substituted aryl, or optionally substituted heteroaryl. In certain embodiments, R^{XA} is optionally substituted alkyl or optionally substituted carbocyclyl. In certain embodiments, R^{XA} is methyl, ethyl, n-propyl, iso-propyl, cyclopropyl, or cyclobutyl. In certain embodiments, R^{XA} is methyl.

In certain embodiments of Formula (XVII^{B}-a-4), wherein X is —NR^{XN}—, provided is a compound of Formula (XVII^{B}-a-7):

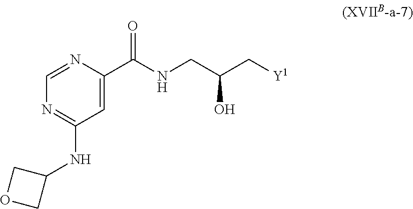

or a pharmaceutically acceptable salt thereof.

In certain embodiments of Formula (XVII^{B}-a-3), wherein a and b are 2, provided is a compound of Formula (XVII^{B}-a-8):

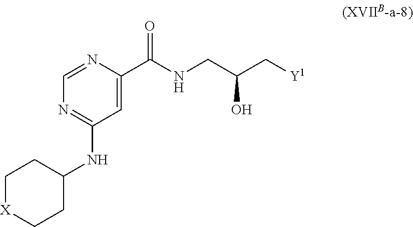

or a pharmaceutically acceptable salt thereof, wherein Y¹ is as generally defined herein, and wherein X is —C(R^{XC})₂—, —O—, —S—, or —NR^{XN}—, wherein each instance of R^{XC} is independently hydrogen, optionally substituted alkyl, optionally substituted carbocyclyl, optionally substituted heterocyclyl, optionally substituted aryl, or optionally substituted heteroaryl; R^{XN} is independently hydrogen, optionally substituted alkyl, optionally substituted carbocyclyl, optionally substituted heterocyclyl, optionally substituted aryl, optionally substituted heteroaryl, —C(═O)R^{XA}, or a nitrogen protecting group; R^{XA} is optionally substituted alkyl, optionally substituted carbocyclyl, optionally substituted heterocyclyl, optionally substituted aryl, or optionally substituted heteroaryl. In certain embodiments, X is —O—. In certain embodiments, X is —NR^{XN}—, wherein R^{XN} is as generally defined above. In certain embodiments, X is —NR^{XN}—, wherein R^{XN} is optionally substituted alkyl, —C(═O)R^{XA}, or a nitrogen protecting group. In certain embodiments, X is —NR^{XN}—, wherein R^{XN} is —C(═O)R^{XA}, wherein R^{XA} is optionally substituted alkyl or optionally substituted carbocyclyl. In certain embodiments, X is —NR^{XN}—, wherein R^{XN} is —C(═O)R^{XA}, wherein R^{XA} is methyl, ethyl, n-propyl, iso-propyl, cyclopropyl, or cyclobutyl. In certain embodiments, X is —NC(═O)CH₃.

In certain embodiments of Formula (XVII^{B}-a-8), wherein X is —NR^{x}—, provided is a compound of Formula (XVII^{B}-a-9):

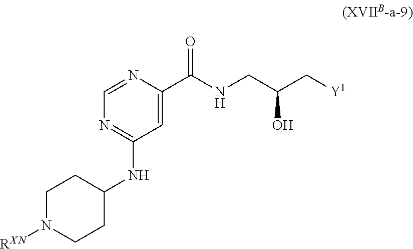

or a pharmaceutically acceptable salt thereof, wherein Y¹ is as generally defined herein, and wherein R^{XN} is independently hydrogen, optionally substituted alkyl, optionally substituted carbocyclyl, optionally substituted heterocyclyl, optionally substituted aryl, optionally substituted heteroaryl, —C(═O)R^{XA}, or a nitrogen protecting group; R^{XA} is optionally substituted alkyl, optionally substituted carbocyclyl, optionally substituted heterocyclyl, optionally substituted aryl, or optionally substituted heteroaryl. In certain embodiments, R^{XN} is optionally substituted alkyl, —C(═O)R^{XA}, or a nitrogen protecting group. In certain embodiments, R^{XN} is —C(═O)R^{XA}, wherein R^{XA} is optionally substituted alkyl or optionally substituted carbocyclyl. In certain embodiments, R^{XN} is —C(═O)R^{XA}, wherein R^{XA} is methyl, ethyl, n-propyl, iso-propyl, cyclopropyl, or cyclobutyl. In certain embodiments, R^{XN} is —C(═O)R^{XA}, wherein R^{XA} is methyl.

In certain embodiments of Formula (XVII^{B}-a-9), wherein —NR^{XN}— is —C(═O)R^{XA} provided is a compound of Formula (XVII^{B}-a-10):

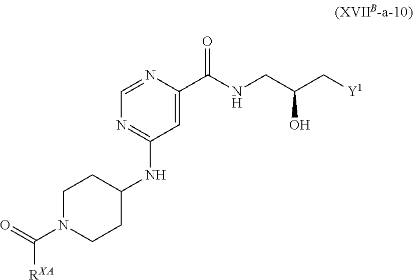

or a pharmaceutically acceptable salt thereof, wherein Y¹ is as generally defined herein, and wherein R^{XA} is optionally substituted alkyl, optionally substituted carbocyclyl, optionally substituted heterocyclyl, optionally substituted aryl, or optionally substituted heteroaryl. In certain embodiments, R^{XA} is optionally substituted alkyl or optionally substituted carbocyclyl. In certain embodiments, R^{XA} is methyl, ethyl, n-propyl, iso-propyl, cyclopropyl, or cyclobutyl. In certain embodiments, R^{XA} is methyl.

In certain embodiments, a provided compound is of Formula (XVII^{B}-b):

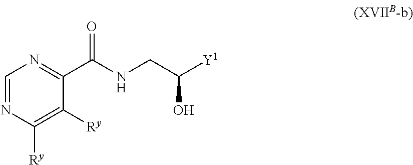

or a pharmaceutically acceptable salt thereof, wherein Y¹ and each instance of R^{y} is as generally defined herein.

In certain embodiments of Formula (XVII^{B}-b), wherein at least one of R^{y} is —N(R^{B})₂, provided is a compound of Formula (XVII^{B}-b-1):

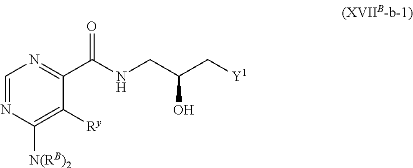

or a pharmaceutically acceptable salt thereof, wherein Y¹, R^{y} and each instance of R are as generally defined herein. In certain embodiments, at least one R^{B} is an optionally substituted carbocyclic ring or optionally substituted heterocyclic ring, e.g., a 4- to 6-membered optionally substituted carbocyclic ring or a 4- to 6-membered optionally substituted heterocyclic ring.

In certain embodiments of Formula (XVII^{B}-b-1), wherein at least one R^{B} is a hydrogen, provided is a compound of Formula (XVII^{B}-b-2):

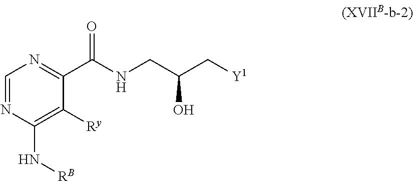

or a pharmaceutically acceptable salt thereof, wherein Y¹, R^{y} and R^{B} are as generally defined herein. In certain embodiments, R^{B} is an optionally substituted carbocyclic ring or optionally substituted heterocyclic ring. In certain embodiments, R^{B} is an optionally substituted carbocyclic ring, e.g., a 4- to 6-membered optionally substituted carbocyclic ring. In certain embodiments, R^{B} is an optionally substituted heterocyclic ring, e.g., or a 4- to 6-membered optionally substituted heterocyclic ring.

In certain embodiments of Formula (XVII^{B}-b-2), wherein R^{B} is an optionally substituted heterocyclic ring, provided is a compound of Formula (XVII^{B}-b-3):

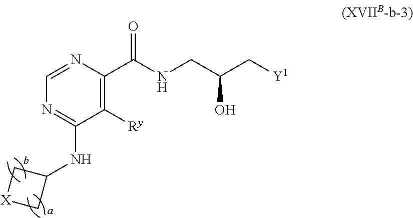

or a pharmaceutically acceptable salt thereof, wherein Y¹ is as generally defined herein, and wherein each instance of a and b is independently 1 or 2, and X is —C(R^{XC})₂—, —O—, —S—, or —NR^{XN}—, wherein each instance of R^{XC} is independently hydrogen, optionally substituted alkyl, optionally substituted carbocyclyl, optionally substituted heterocyclyl, optionally substituted aryl, or optionally substituted heteroaryl; R^{XN} is independently hydrogen, optionally substituted alkyl, optionally substituted carbocyclyl, optionally substituted heterocyclyl, optionally substituted aryl, optionally substituted heteroaryl, —C(═O)R^{XA}, or a nitrogen protecting group; R^{XA} is optionally substituted alkyl, optionally substituted carbocyclyl, optionally substituted heterocyclyl, optionally substituted aryl, or optionally substituted heteroaryl. In certain embodiments, a and b are both 1. In certain embodiments, a and b are both 2. In certain embodiments, X is —O—. In certain embodiments, X is —NR^{XN}—, wherein R^{XN} is as generally defined above. In certain embodiments, X is —NR^{XN}—, wherein R^{XN} is optionally substituted alkyl, —C(═O)R^{XA}, or a nitrogen protecting group. In certain embodiments, X is —NR^{XN}—, wherein R^{XN} is —C(═O)R^{A}, wherein R^{XA} is optionally substituted alkyl or optionally substituted carbocyclyl. In certain embodiments, X is —NR^{XN}—, wherein R^{XN} is —C(═O)R^{XA}, wherein R^{XA} is methyl, ethyl, n-propyl, iso-propyl, cyclopropyl, or cyclobutyl. In certain embodiments, a and b are each independently 1 or 2; and X is —O— or —NR^{XN}—, wherein R^{XN} is as generally defined above. In certain embodiments, a and b are each independently 1 or 2; and X is —O— or —NC(═O)R^{XA}, wherein R^{XA} is as generally defined above. In certain embodiments, a and b are both 1; and X is —O— or —NR^{XN}—, wherein R^{XN} is as generally defined above. In certain embodiments, a and b are both 1; and X is —O— or —NC(═O)R^{XA}, wherein R^{XA} is as generally defined above. In certain embodiments, a and b are both 1; and X is —O— or —NC(═O)CH₃. In certain embodiments, a and b are both 1; and X is —O—. In certain embodiments, a and b are both 2; and X is —O— or —NC(═O)CH₃. In certain embodiments, a and b are both 2; and X is —NC(═O)CH₃.

In certain embodiments, a provided compound is of Formula (XV^{B}-b):

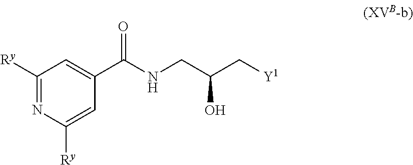

or a pharmaceutically acceptable salt thereof, wherein Y¹ and each R^{y} is as generally described herein.

In certain embodiments of Formula (XV^{B}-b), wherein at least one of R^{y} is —N(R^{B})₂, provided is a compound of Formula (XV^{B}-b-1):

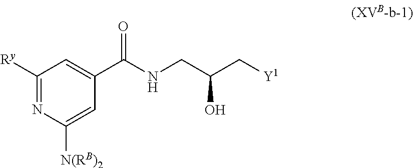

or a pharmaceutically acceptable salt thereof, wherein Y¹, R^{y} and R^{B} are as generally described herein. In certain embodiments, at least one R^{B} is an optionally substituted carbocyclic ring or optionally substituted heterocyclic ring, e.g., a 4- to 6-membered optionally substituted carbocyclic ring or a 4- to 6-membered optionally substituted heterocyclic ring.

In certain embodiments of Formula (XV^{B}-b-1), wherein at least one R^{B} is a hydrogen, provided is a compound of Formula (XV^{B}-b-2):

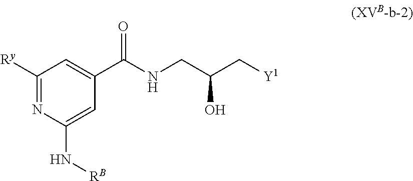

or a pharmaceutically acceptable salt thereof, wherein Y¹, R^{y} and R^{B} are as generally described herein. In certain embodiments, R^{B} is an optionally substituted carbocyclic ring or optionally substituted heterocyclic ring. In certain embodiments, R^{B} is an optionally substituted carbocyclic ring, e.g., a 4- to 6-membered optionally substituted carbocyclic ring. In certain embodiments, R^{B} is an optionally substituted heterocyclic ring, e.g., or a 4- to 6-membered optionally substituted heterocyclic ring.

In certain embodiments of Formula (XV^{B}-b-2), wherein R^{B} is an optionally substituted heterocyclic ring, provided is a compound of Formula (XV^{B}-b-3):

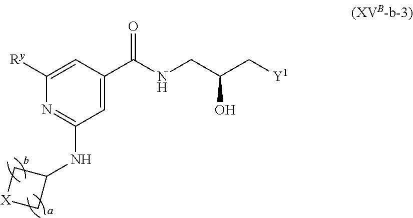

or a pharmaceutically acceptable salt thereof, wherein Y¹ is as generally defined herein, and wherein each instance of a and b is independently 1 or 2, and X is —C(R^{XC})₂—, —O—, —S—, or —NR^{XN}—, wherein each instance of R^{XC} is independently hydrogen, optionally substituted alkyl, optionally substituted carbocyclyl, optionally substituted heterocyclyl, optionally substituted aryl, or optionally substituted heteroaryl; R^{XN} is independently hydrogen, optionally substituted alkyl, optionally substituted carbocyclyl, optionally substituted heterocyclyl, optionally substituted aryl, optionally substituted heteroaryl, —C(═O)R^{XA}, or a nitrogen protecting group; R^{XA} is optionally substituted alkyl, optionally substituted carbocyclyl, optionally substituted heterocyclyl, optionally substituted aryl, or optionally substituted heteroaryl. In certain embodiments, a and b are both 1. In certain embodiments, a and b are both 2. In certain embodiments, X is —O—. In certain embodiments, X is —NR^{XN}—, wherein R^{XN} is as generally defined above. In certain embodiments, X is —NR—N, wherein R^{XN} is optionally substituted alkyl, —C(═O)R^{XA}, or a nitrogen protecting group. In certain embodiments, X is —NR^{XN}—, wherein R^{XN} is —C(═O)R^{A}, wherein R^{XA} is optionally substituted alkyl or optionally substituted carbocyclyl. In certain embodiments, X is —NR^{XN}—, wherein R^{XN} is —C(═O)R^{XN}, wherein R^{XA} is methyl, ethyl, n-propyl, iso-propyl, cyclopropyl, or cyclobutyl. In certain embodiments, a and b are each independently 1 or 2; and X is —O— or —NR^{XN}—, wherein R^{x} is as generally defined above. In certain embodiments, a and b are each independently 1 or 2; and X is —O— or —NC(═O)R^{XA}, wherein R^{XA} is as generally defined above. In certain embodiments, a and b are both 1; and X is —O— or —NR^{XN}—, wherein R^{XN} is as generally defined above. In certain embodiments, a and b are both 1; and X is —O— or —NC(═O)CH₃. In certain embodiments, a and b are both 1; and X is —O—. In certain embodiments, a and b are both 2; and X is —O— or —NC(═O)CH₃. In certain embodiments, a and b are both 2; and X is —NC(═O)CH₃.

In certain embodiments, a provided compound is of Formula (XV^{B}-c):

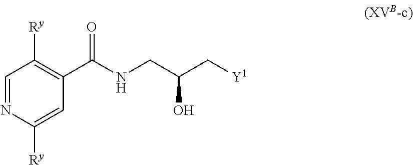

or a pharmaceutically acceptable salt thereof, wherein Y¹ and each R^{y} is as generally described herein.

In certain embodiments of Formula (XV^{B}-c), wherein at least one of R^{y} is —N(R^{B})₂, provided is a compound of Formula (XV^{B}-c-1):

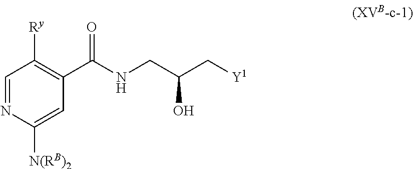

or a pharmaceutically acceptable salt thereof, wherein Y¹, R^{y} and R^{B} are as generally described herein. In certain embodiments, at least one R^{B} is an optionally substituted carbocyclic ring or optionally substituted heterocyclic ring, e.g., a 4- to 6-membered optionally substituted carbocyclic ring or a 4- to 6-membered optionally substituted heterocyclic ring.

In certain embodiments of Formula (XV^{B}-c-1), wherein at least one R^{B} is a hydrogen, provided is a compound of Formula (XV^{B}-c-2):

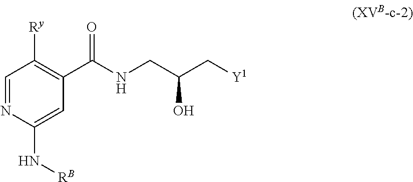

or a pharmaceutically acceptable salt thereof, wherein Y¹, R^{y} and R^{B} are as generally described herein. In certain embodiments, R^{B} is an optionally substituted carbocyclic ring or optionally substituted heterocyclic ring. In certain embodiments, R^{B} is an optionally substituted carbocyclic ring, e.g., a 4- to 6-membered optionally substituted carbocyclic ring. In certain embodiments, R^{B} is an optionally substituted heterocyclic ring, e.g., or a 4- to 6-membered optionally substituted heterocyclic ring.

In certain embodiments of Formula (XV^{B}-c-2), wherein R^{B} is an optionally substituted heterocyclic ring, provided is a compound of Formula (XV^{B}-c-3):

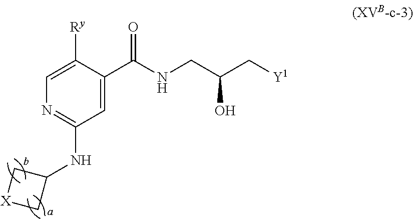

or a pharmaceutically acceptable salt thereof, wherein Y¹ is as generally defined herein, and wherein each instance of a and b is independently 1 or 2, and X is —C(R^{XC})₂—, —O—, —S—, or —NR^{XN}—, wherein each instance of R^{XC} is independently hydrogen, optionally substituted alkyl, optionally substituted carbocyclyl, optionally substituted heterocyclyl, optionally substituted aryl, or optionally substituted heteroaryl; R^{XN} is independently hydrogen, optionally substituted alkyl, optionally substituted carbocyclyl, optionally substituted heterocyclyl, optionally substituted aryl, optionally substituted heteroaryl, —C(═O)R^{XA}, or a nitrogen protecting group; R^{XA} is optionally substituted alkyl, optionally substituted carbocyclyl, optionally substituted heterocyclyl, optionally substituted aryl, or optionally substituted heteroaryl. In certain embodiments, a and b are both 1. In certain embodiments, a and b are both 2. In certain embodiments, X is —O—. In certain embodiments, X is —NR^{XN}—, wherein R^{XN} is as generally defined herein. In certain embodiments, X is —NR^{XN}—, wherein R^{XN} is optionally substituted alkyl, —C(═O)R^{XA}, or a nitrogen protecting group. In certain embodiments, X is —NR^{XN}—, wherein R^{XN} is —C(═O)R^{A}, wherein R^{XA} is optionally substituted alkyl or optionally substituted carbocyclyl. In certain embodiments, X is —NR^{XN}—, wherein R^{XN} is —C(═O)R^{x}, wherein R^{XA} is methyl, ethyl, n-propyl, iso-propyl, cyclopropyl, or cyclobutyl. In certain embodiments, a and b are each independently 1 or 2; and X is —O— or —NR^{XN}—, wherein R^{XN} is as generally defined herein. In certain embodiments, a and b are each independently 1 or 2; and X is —O— or —NC(═O)R^{XA}, wherein R^{XA} is as generally defined herein. In certain embodiments, a and b are both 1; and X is —O— or —NR—N, wherein R^{XN} is as generally defined herein. In certain embodiments, a and b are both 1; and X is —O— or —NC(═O)CH₃. In certain embodiments, a and b are both 1; and X is —O—. In certain embodiments, a and b are both 2; and X is —O— or —NC(═O)CH₃. In certain embodiments, a and b are both 2; and X is —NC(═O)CH₃.

In certain embodiments, a provided compound is a compound listed in Table 1A, or a pharmaceutically acceptable salt thereof.

TABLE 1A
Exemplary Compounds

| # | Compound | Exact Mass | LCMS |
|---|---|---|---|
| 1-1 | 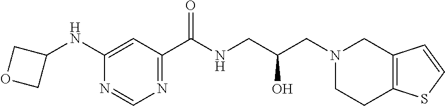 [SMILES: O=C(NC[C@H](O)CN1CCc2sccc2C1)c1cc(NC2COC2)ncn1] | 389.1522 | 390.1 |
| 1-2 | 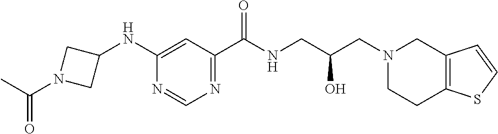 [SMILES: CC(=O)N1CC(Nc2cc(C(=O)NC[C@H](O)CN3CCc4sccc4C3)ncn2)C1] | 430.1787 | 431.2 |
| 1-3 | 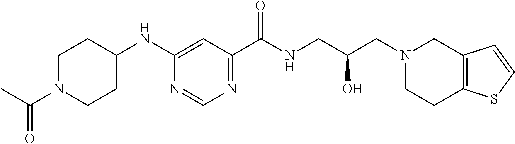 [SMILES: CC(=O)N1CCC(Nc2cc(C(=O)NC[C@H](O)CN3CCc4sccc4C3)ncn2)CC1] | 458.21 | 459.2 |
| 1-4 | 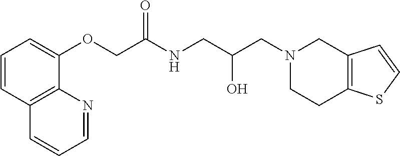 [SMILES: O=C(COc1cccc2cccnc12)NCC(O)CN1CCc2sccc2C1] | 397.146 | 398.2 |
| 1-5 | 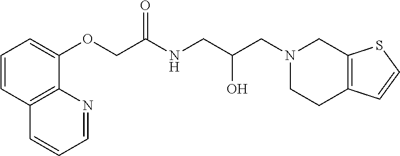 [SMILES: O=C(COc1cccc2cccnc12)NCC(O)CN1CCc2ccsc2C1] | 397.146 | 398.2 |
| 1-6 | 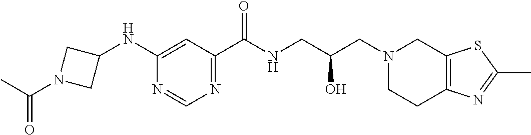 [SMILES: CC(=O)N1CC(Nc2cc(C(=O)NC[C@H](O)CN3CCc4nc(C)sc4C3)ncn2)C1] | 445.1896 | 446.2 |
| 1-7 | 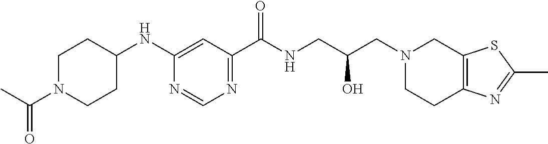 [SMILES: CC(=O)N1CCC(Nc2cc(C(=O)NC[C@H](O)CN3CCc4nc(C)sc4C3)ncn2)CC1] | 473.2209 | 474.3 |
| 1-8 | 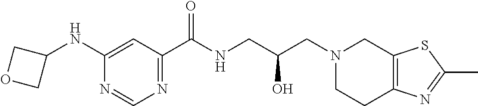 [SMILES: Cc1nc2c(s1)CN(C[C@@H](O)CNC(=O)c1cc(NC3COC3)ncn1)CC2] | 404.1631 | 405.2 |
| 1-9 | 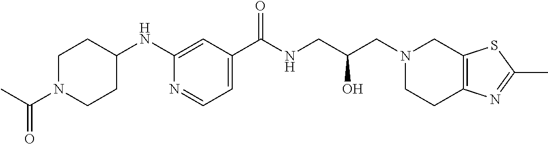 [SMILES: CC(=O)N1CCC(Nc2cc(C(=O)NC[C@H](O)CN3CCc4nc(C)sc4C3)ccn2)CC1] | 472.2257 | 473.2 |
| 1-10 | 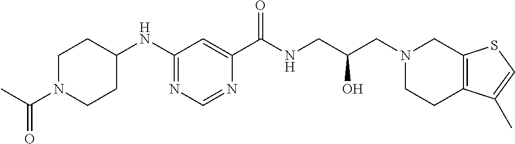 [SMILES: CC(=O)N1CCC(Nc2cc(C(=O)NC[C@H](O)CN3CCc4c(C)csc4C3)ncn2)CC1] | 472.2257 | 473.2 |
| 1-11 | 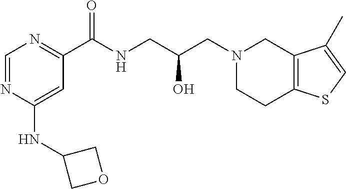 [SMILES: Cc1csc2c1CN(C[C@@H](O)CNC(=O)c1cc(NC3COC3)ncn1)CC2] | 403.1678 | 404.1 |
| 1-12 | 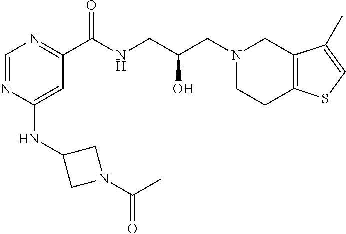 [SMILES: CC(=O)N1CC(Nc2cc(C(=O)NC[C@H](O)CN3CCc4scc(C)c4C3)ncn2)C1] | 444.1944 | 445.2 |
| 1-13 | 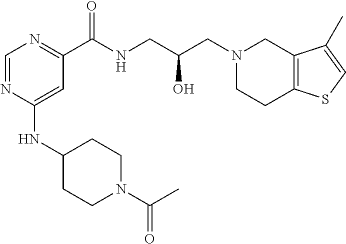 [SMILES: CC(=O)N1CCC(Nc2cc(C(=O)NC[C@H](O)CN3CCc4scc(C)c4C3)ncn2)CC1] | 472.2257 | 473.2 |
| 1-14 | 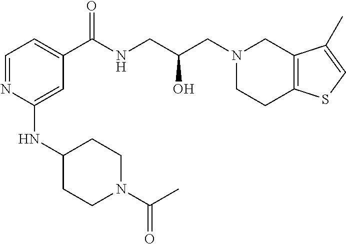 [SMILES: CC(=O)N1CCC(Nc2cc(C(=O)NC[C@H](O)CN3CCc4scc(C)c4C3)ccn2)CC1] | 471.2304 | 472.3 |
| 1-15 | 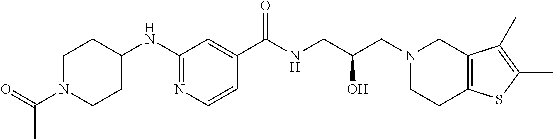 [SMILES: CC(=O)N1CCC(Nc2cc(C(=O)NC[C@H](O)CN3CCc4sc(C)c(C)c4C3)ccn2)CC1] | 485.2461 | 486.3 |
| 1-16 | 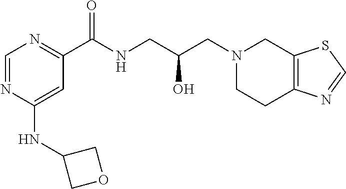 [SMILES: O=C(NC[C@H](O)CN1CCc2ncsc2C1)c1cc(NC2COC2)ncn1] | 390.1474 | 391.2 |
| 1-17 | 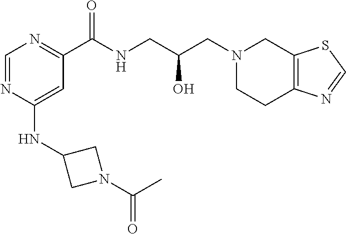 [SMILES: CC(=O)N1CC(Nc2cc(C(=O)NC[C@H](O)CN3CCc4ncsc4C3)ncn2)C1] | 431.1740 | 432.2 |
| 1-18 | 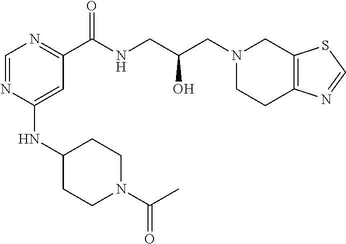 [SMILES: CC(=O)N1CCC(Nc2cc(C(=O)NC[C@H](O)CN3CCc4ncsc4C3)ncn2)CC1] | 459.2053 | 460.2 |
| 1-19 | 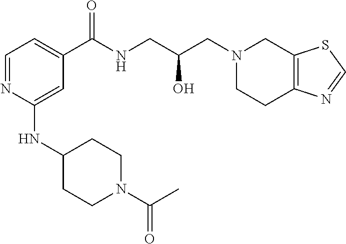 [SMILES: CC(=O)N1CCC(Nc2cc(C(=O)NC[C@H](O)CN3CCc4ncsc4C3)ccn2)CC1] | 458.2100 | 459.1 |
| 1-20 | 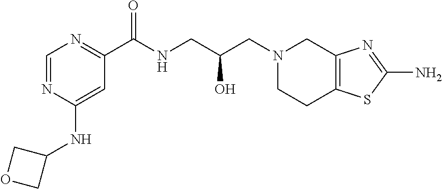 [SMILES: Nc1nc2c(s1)CCN(C[C@@H](O)CNC(=O)c1cc(NC3COC3)ncn1)C2] | 405.1583 | 406.2 |
| 1-21 | 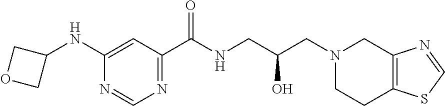 [SMILES: O=C(NC[C@H](O)CN1CCc2scnc2C1)c1cc(NC2COC2)ncn1] | 390.1474 | 391.1 |
| 1-22 | 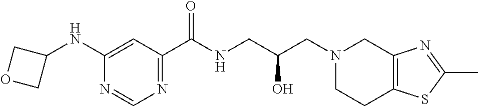 [SMILES: Cc1nc2c(s1)CCN(C[C@@H](O)CNC(=O)c1cc(NC3COC3)ncn1)C2] | 404.1631 | 405.2 |
| 1-23 | 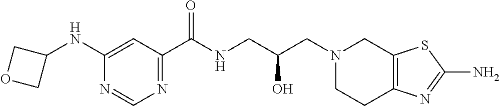 [SMILES: Nc1nc2c(s1)CN(C[C@@H](O)CNC(=O)c1cc(NC3COC3)ncn1)CC2] | 405.1583 | 406.2 |
| 1-24 | 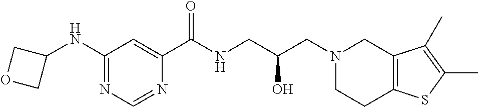 [SMILES: Cc1sc2c(c1C)CN(C[C@@H](O)CNC(=O)c1cc(NC3COC3)ncn1)CC2] | 417.1835 | 418.2 |
| 1-25 | 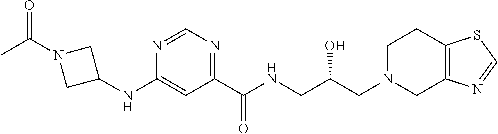 [SMILES: CC(=O)N1CC(Nc2cc(C(=O)NC[C@H](O)CN3CCc4scnc4C3)ncn2)C1] | 431.174 | 432.1 |
| 1-26 | 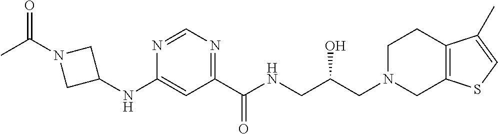 [SMILES: CC(=O)N1CC(Nc2cc(C(=O)NC[C@H](O)CN3CCc4c(C)csc4C3)ncn2)C1] | 444.1944 | 445.1 |
| 1-27 | 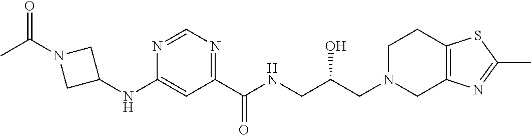 [SMILES: CC(=O)N1CC(Nc2cc(C(=O)NC[C@H](O)CN3CCc4sc(C)nc4C3)ncn2)C1] | 445.1896 | 446.2 |
| 1-28 | 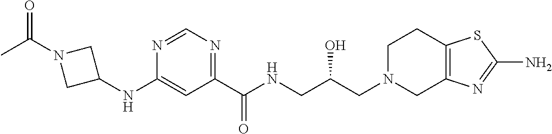 [SMILES: CC(=O)N1CC(Nc2cc(C(=O)NC[C@H](O)CN3CCc4sc(N)nc4C3)ncn2)C1] | 446.1849 | 447.2 |
| 1-29 | 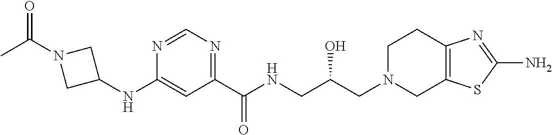 [SMILES: CC(=O)N1CC(Nc2cc(C(=O)NC[C@H](O)CN3CCc4nc(N)sc4C3)ncn2)C1] | 446.1849 | 447.2 |
| 1-30 | 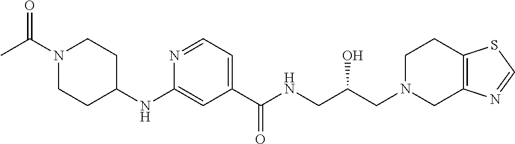 [SMILES: CC(=O)N1CCC(Nc2cc(C(=O)NC[C@H](O)CN3CCc4scnc4C3)ccn2)CC1] | 458.21 | 459.2 |
| 1-31 | 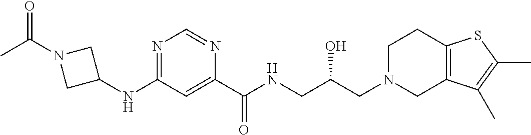 [SMILES: CC(=O)N1CC(Nc2cc(C(=O)NC[C@H](O)CN3CCc4sc(C)c(C)c4C3)ncn2)C1] | 458.21 | 459.2 |
| 1-32 | 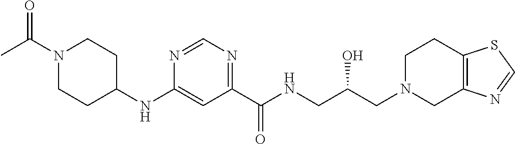 [SMILES: CC(=O)N1CCC(Nc2cc(C(=O)NC[C@H](O)CN3CCc4scnc4C3)ncn2)CC1] | 459.2053 | 460.2 |
| 1-33 | 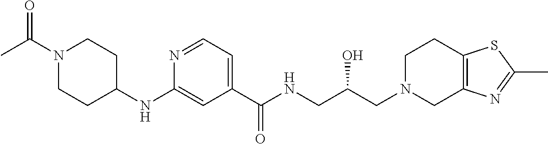 [SMILES: CC(=O)N1CCC(Nc2cc(C(=O)NC[C@H](O)CN3CCc4sc(C)nc4C3)ccn2)CC1] | 472.2257 | 473.3 |
| 1-34 | 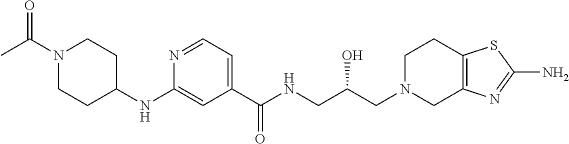 [SMILES: CC(=O)N1CCC(Nc2cc(C(=O)NC[C@H](O)CN3CCc4sc(N)nc4C3)ccn2)CC1] | 473.2209 | 474.2 |
| 1-35 | 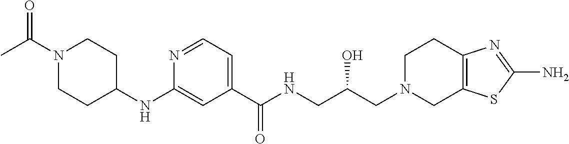 [SMILES: CC(=O)N1CCC(Nc2cc(C(=O)NC[C@H](O)CN3CCc4nc(N)sc4C3)ccn2)CC1] | 473.2209 | 474.2 |
| 1-36 | 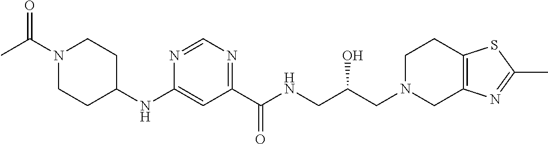 [SMILES: CC(=O)N1CCC(Nc2cc(C(=O)NC[C@H](O)CN3CCc4sc(C)nc4C3)ncn2)CC1] | 473.2209 | 474.3 |
| 1-37 | 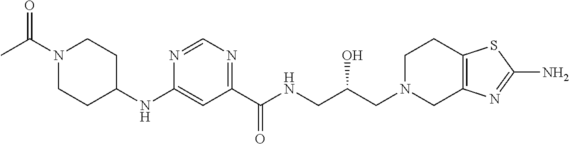 [SMILES: CC(=O)N1CCC(Nc2cc(C(=O)NC[C@H](O)CN3CCc4sc(N)nc4C3)ncn2)CC1] | 474.2162 | 475.3 |
| 1-38 | 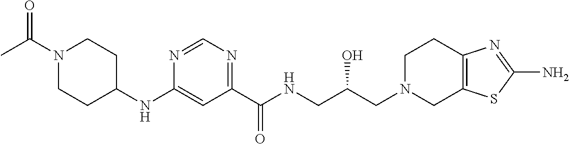 [SMILES: CC(=O)N1CCC(Nc2cc(C(=O)NC[C@H](O)CN3CCc4nc(N)sc4C3)ncn2)CC1] | 474.2162 | 475.2 |
| 1-39 | 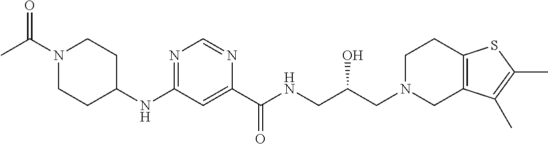 [SMILES: CC(=O)N1CCC(Nc2cc(C(=O)NC[C@H](O)CN3CCc4sc(C)c(C)c4C3)ncn2)CC1] | 486.2413 | 487.3 |

In certain embodiments, a provided compound is a compound listed in Table 1B, or a pharmaceutically acceptable salt thereof.

TABLE 1B
Exemplary Compounds

| | | Exact | |
|---|---|---|---|
| # | Compound | Mass | LCMS |
| 2-1 | 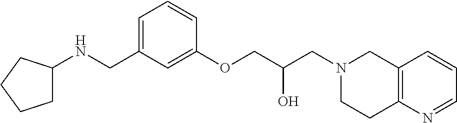 [SMILES: OC(COc1cccc(CNC2CCCC2)c1)CN1CCc2ncccc2C1] | 381.2416 | 382.0 |
| 2-2 | 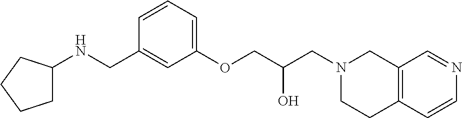 [SMILES: OC(COc1cccc(CNC2CCCC2)c1)CN1CCc2ccncc2C1] | 381.2416 | 382.1 |
| 2-3 | 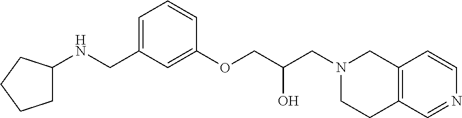 [SMILES: OC(COc1cccc(CNC2CCCC2)c1)CN1CCc2cnccc2C1] | 381.2416 | 382.2 |
| 2-4 | 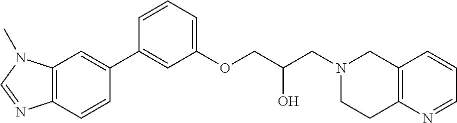 [SMILES: Cn1cnc2ccc(-c3cccc(OCC(O)CN4CCc5ncccc5C4)c3)cc21] | 414.2056 | 415.2 |
| 2-5 | 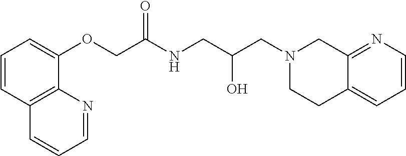 [SMILES: O=C(COc1cccc2cccnc12)NCC(O)CN1CCc2cccnc2C1] | 392.1848 | 393.2 |
| 2-6 | 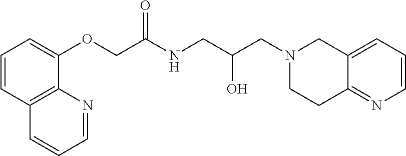 [SMILES: O=C(COc1cccc2cccnc12)NCC(O)CN1CCc2ncccc2C1] | 392.1848 | 393.2 |
| 2-7 | 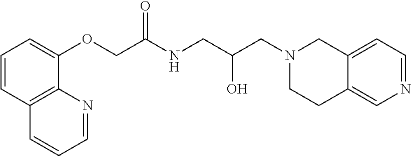 [SMILES: O=C(COc1cccc2cccnc12)NCC(O)CN1CCc2cnccc2C1] | 392.1848 | 393.3 |
| 2-8 | 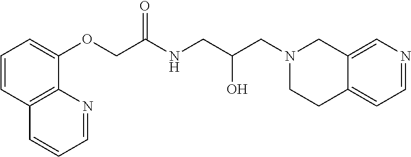 [SMILES: O=C(COc1cccc2cccnc12)NCC(O)CN1CCc2ccncc2C1] | 392.1848 | 393.3 |
| 2-9 | 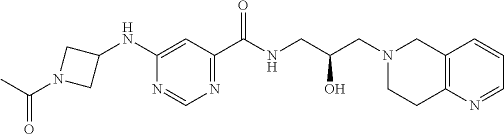 [SMILES: CC(=O)N1CC(Nc2cc(C(=O)NC[C@H](O)CN3CCc4ncccc4C3)ncn2)C1] | 425.2175 | 426.2 |
| 2-10 | 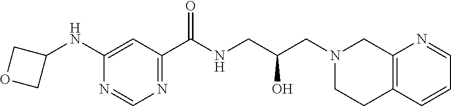 [SMILES: O=C(NC[C@H](O)CN1CCc2cccnc2C1)c1cc(NC2COC2)ncn1] | 384.191 | 385.0 |
| 2-11 | 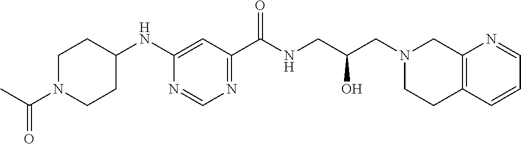 [SMILES: CC(=O)N1CCC(Nc2cc(C(=O)NC[C@H](O)CN3CCc4cccnc4C3)ncn2)CC1] | 453.2488 | 454.1 |
| 2-12 | 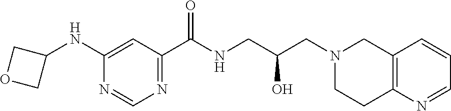 [SMILES: O=C(NC[C@H](O)CN1CCc2ncccc2C1)c1cc(NC2COC2)ncn1] | 384.191 | 385.1 |
| 2-13 | 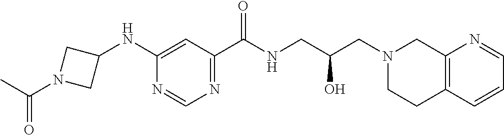 [SMILES: CC(=O)N1CC(Nc2cc(C(=O)NC[C@H](O)CN3CCc4cccnc4C3)ncn2)C1] | 425.2175 | 426.1 |
| 2-14 | 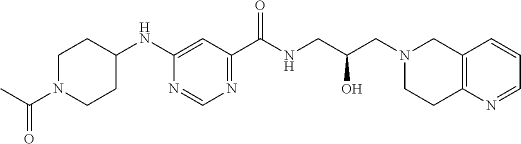 [SMILES: CC(=O)N1CCC(Nc2cc(C(=O)NC[C@H](O)CN3CCc4ncccc4C3)ncn2)CC1] | 453.2488 | 454.2 |
| 2-15 | 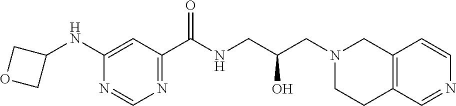 [SMILES: O=C(NC[C@H](O)CN1CCc2cnccc2C1)c1cc(NC2COC2)ncn1] | 384.191 | 385.0 |
| 2-16 | 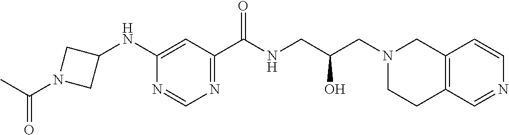 [SMILES: CC(=O)N1CC(Nc2cc(C(=O)NC[C@H](O)CN3CCc4cnccc4C3)ncn2)C1] | 425.2175 | 426.2 |
| 2-17 | 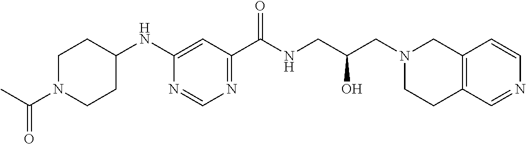 [SMILES: CC(=O)N1CCC(Nc2cc(C(=O)NC[C@H](O)CN3CCc4cnccc4C3)ncn2)CC1] | 453.2488 | 454.2 |
| 2-18 | 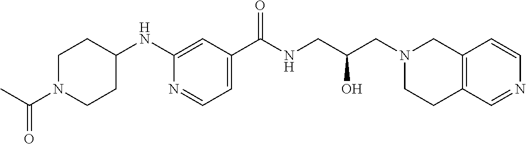 [SMILES: CC(=O)N1CCC(Nc2cc(C(=O)NC[C@H](O)CN3CCc4cnccc4C3)ccn2)CC1] | 452.2536 | 453.3 |
| 2-19 | 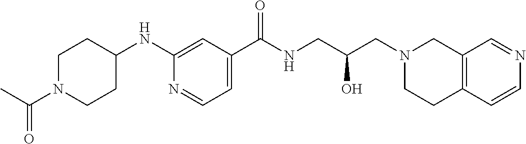 [SMILES: CC(=O)N1CCC(Nc2cc(C(=O)NC[C@H](O)CN3CCc4ccncc4C3)ccn2)CC1] | 452.2536 | 453.3 |
| 2-20 | 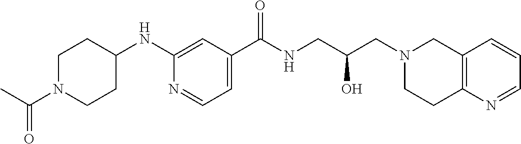 [SMILES: CC(=O)N1CCC(Nc2cc(C(=O)NC[C@H](O)CN3CCc4ncccc4C3)ccn2)CC1] | 452.2536 | 453.3 |
| 2-21 | 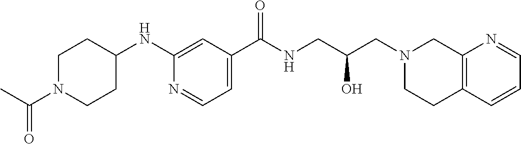 [SMILES: CC(=O)N1CCC(Nc2cc(C(=O)NC[C@H](O)CN3CCc4cccnc4C3)ccn2)CC1] | 452.2536 | 453.3 |
| 2-22 | 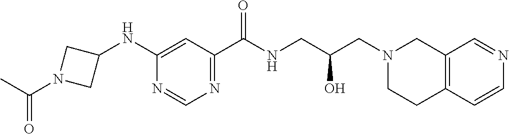 [SMILES: CC(=O)N1CC(Nc2cc(C(=O)NC[C@H](O)CN3CCc4ccncc4C3)ncn2)C1] | 425.2175 | 426.2 |
| 2-23 | 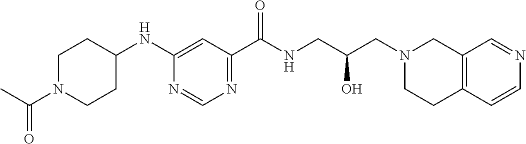 [SMILES: CC(=O)N1CCC(Nc2cc(C(=O)NC[C@H](O)CN3CCc4ccncc4C3)ncn2)CC1] | 453.2488 | 454.3 |
| 2-24 | 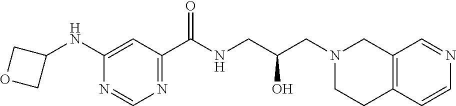 [SMILES: O=C(NC[C@H](O)CN1CCc2ccncc2C1)c1cc(NC2COC2)ncn1] | 384.191 | 385.2 |

In certain embodiments, a provided compound is a compound listed in Table 1C, or a pharmaceutically acceptable salt thereof.

TABLE 1C
Exemplary Compounds

| # | Compound | Exact Mass | LCMS |
|---|---|---|---|
| 3-1 | 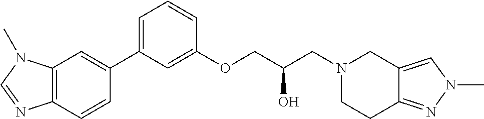 [SMILES: Cn1cc2c(n1)CCN(C[C@@H](O)COc1cccc(-c3ccc4ncn(C)c4c3)c1)C2] | 417.2165 | 418.2 |
| 3-2 | 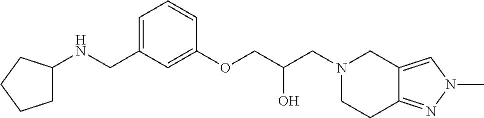 [SMILES: Cn1cc2c(n1)CCN(CC(O)COc1cccc(CNC3CCCC3)c1)C2] | 384.2525 | 385.3 |
| 3-3 | 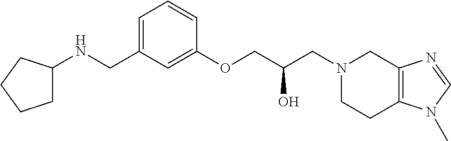 [SMILES: Cn1cnc2c1CCN(C[C@@H](O)COc1cccc(CNC3CCCC3)c1)C2] | 384.2525 | 385.2 |
| 3-4 | 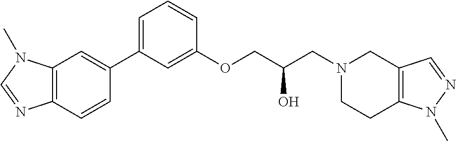 [SMILES: Cn1ncc2c1CCN(C[C@@H](O)COc1cccc(-c3ccc4ncn(C)c4c3)c1)C2] | 417.2165 | 418.3 |
| 3-5 | 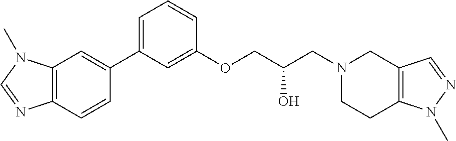 [SMILES: Cn1ncc2c1CCN(C[C@H](O)COc1cccc(-c3ccc4ncn(C)c4c3)c1)C2] | 417.2165 | 418.3 |
| 3-6 | 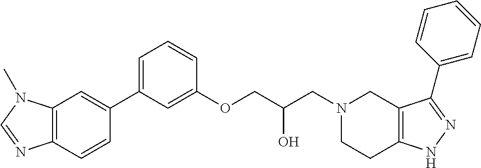 [SMILES: Cn1cnc2ccc(-c3cccc(OCC(O)CN4CCc5[nH]nc(-c6ccccc6)c5C4)c3)cc21] | 479.2321 | 480.3 |
| 3-7 | 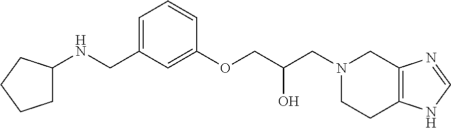 [SMILES: OC(COc1cccc(CNC2CCCC2)c1)CN1CCc2[nH]cnc2C1] | 370.2369 | 371.0 |
| 3-8 | 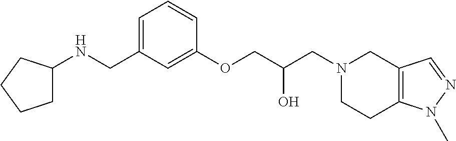 [SMILES: Cn1ncc2c1CCN(CC(O)COc1cccc(CNC3CCCC3)c1)C2] | 384.2525 | 385.3 |
| 3-9 | 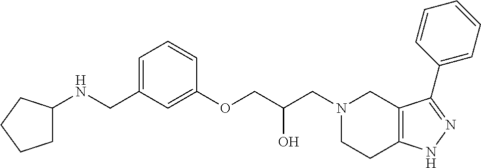 [SMILES: OC(COc1cccc(CNC2CCCC2)c1)CN1CCc2[nH]nc(-c3ccccc3)c2C1] | 446.2682 | 446.9 |
| 3-10 | 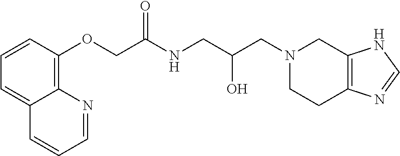 [SMILES: O=C(COc1cccc2cccnc12)NCC(O)CN1CCc2nc[nH]c2C1] | 381.1801 | 382.2 |
| 3-11 | 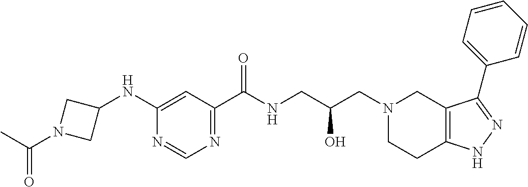 [SMILES: CC(=O)N1CC(Nc2cc(C(=O)NC[C@H](O)CN3CCc4[nH]nc(-c5ccccc5)c4C3)ncn2)C1] | 490.2441 | 491.2 |
| 3-12 | 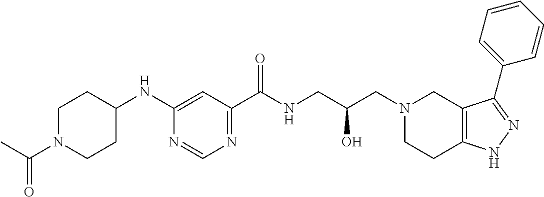 [SMILES: CC(=O)N1CCC(Nc2cc(C(=O)NC[C@H](O)CN3CCc4[nH]nc(-c5ccccc5)c4C3)ncn2)CC1] | 518.2754 | 519.2 |
| 3-13 | 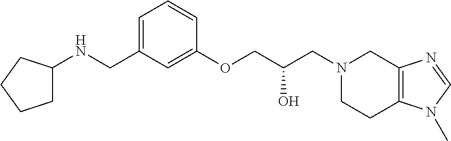 [SMILES: Cn1cnc2c1CCN(C[C@H](O)COc1cccc(CNC3CCCC3)c1)C2] | 384.2525 | 385.2 |
| 3-14 | 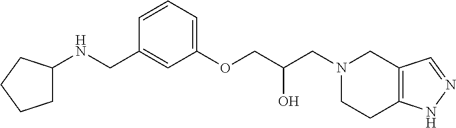 [SMILES: OC(COc1cccc(CNC2CCCC2)c1)CN1CCc2[nH]ncc2C1] | 370.2369 | 371.1 |
| 3-15 | 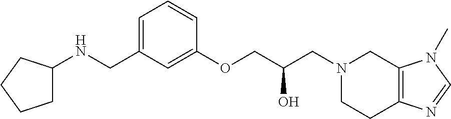 [SMILES: Cn1cnc2c1CN(C[C@@H](O)COc1cccc(CNC3CCCC3)c1)CC2] | 384.2525 | 385.2 |
| 3-16 | 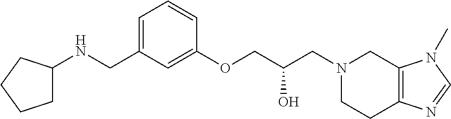 [SMILES: Cn1cnc2c1CN(C[C@H](O)COc1cccc(CNC3CCCC3)c1)CC2] | 384.2525 | 385.2 |
| 3-17 | 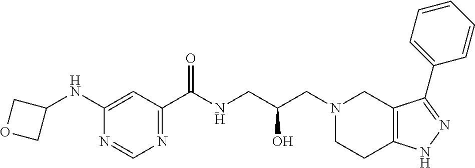 [SMILES: O=C(NC[C@H](O)CN1CCc2[nH]nc(-c3ccccc3)c2C1)c1cc(NC2COC2)ncn1] | 449.2175 | 450.2 |
| 3-18 | 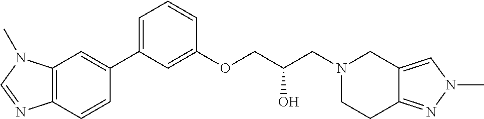 [SMILES: Cn1cc2c(n1)CCN(C[C@H](O)COc1cccc(-c3ccc4ncn(C)c4c3)c1)C2] | 417.2165 | 418.3 |
| 3-19 | 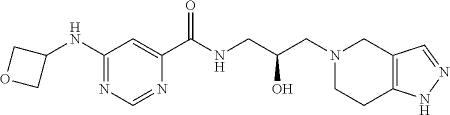 [SMILES: O=C(NC[C@H](O)CN1CCc2[nH]ncc2C1)c1cc(NC2COC2)ncn1] | 373.1862 | 374.2 |
| 3-20 | 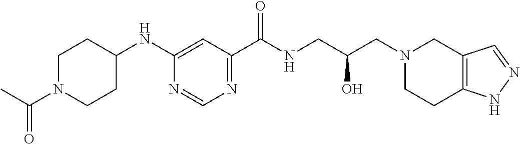 [SMILES: CC(=O)N1CCC(Nc2cc(C(=O)NC[C@H](O)CN3CCc4[nH]ncc4C3)ncn2)CC1] | 442.2441 | 443.3 |
| 3-21 | 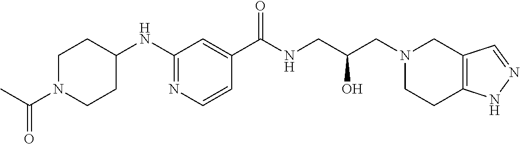 [SMILES: CC(=O)N1CCC(Nc2cc(C(=O)NC[C@H](O)CN3CCc4[nH]ncc4C3)ccn2)CC1] | 441.2488 | 442.3 |
| 3-22 | 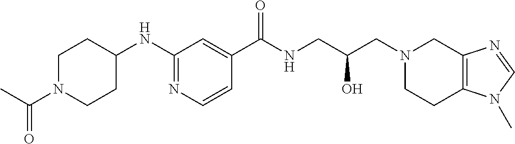 [SMILES: CC(=O)N1CCC(Nc2cc(C(=O)NC[C@H](O)CN3CCc4c(ncn4C)C3)ccn2)CC1] | 455.2645 | 456.3 |
| 3-23 | 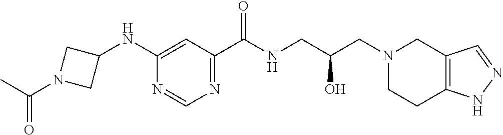 [SMILES: CC(=O)N1CC(Nc2cc(C(=O)NC[C@H](O)CN3CCc4[nH]ncc4C3)ncn2)C1] | 414.2128 | 415.2 |
| 3-24 | 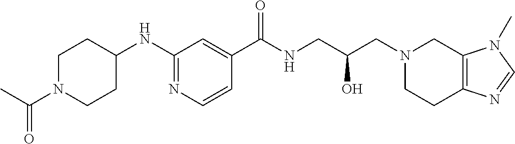 [SMILES: CC(=O)N1CCC(Nc2cc(C(=O)NC[C@H](O)CN3CCc4ncn(C)c4C3)ccn2)CC1] | 455.2645 | 456.3 |
| 3-25 | 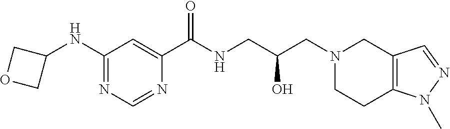 [SMILES: Cn1ncc2c1CCN(C[C@@H](O)CNC(=O)c1cc(NC3COC3)ncn1)C2] | 387.2019 | 388.1 |
| 3-26 | 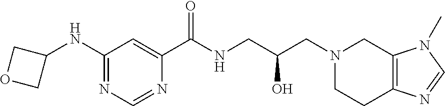 [SMILES: Cn1cnc2c1CN(C[C@@H](O)CNC(=O)c1cc(NC3COC3)ncn1)CC2] | 387.2019 | 388.2 |
| 3-27 | 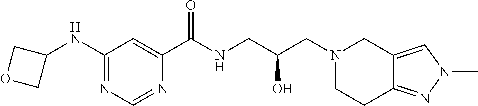 [SMILES: Cn1cc2c(n1)CCN(C[C@@H](O)CNC(=O)c1cc(NC3COC3)ncn1)C2] | 387.2019 | 388.1 |
| 3-28 | 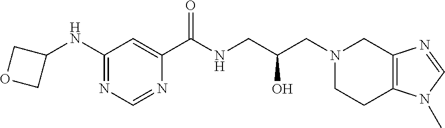 [SMILES: Cn1cnc2c1CCN(C[C@@H](O)CNC(=O)c1cc(NC3COC3)ncn1)C2] | 387.2019 | 388.2 |
| 3-29 | 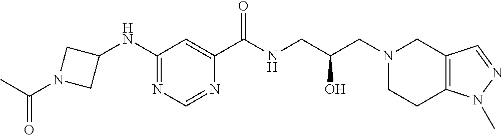 [SMILES: CC(=O)N1CC(Nc2cc(C(=O)NC[C@H](O)CN3CCc4c(cnn4C)C3)ncn2)C1] | 428.2284 | 429.2 |
| 3-30 | 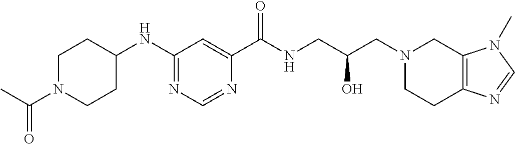 [SMILES: CC(=O)N1CCC(Nc2cc(C(=O)NC[C@H](O)CN3CCc4ncn(C)c4C3)ncn2)CC1] | 456.2597 | 457.3 |
| 3-31 | 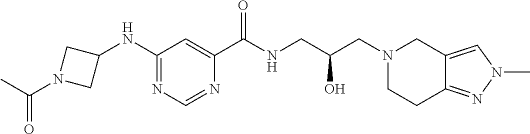 [SMILES: CC(=O)N1CC(Nc2cc(C(=O)NC[C@H](O)CN3CCc4nn(C)cc4C3)ncn2)C1] | 428.2284 | 429.2 |
| 3-32 | 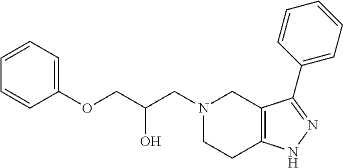 [SMILES: OC(COc1ccccc1)CN1CCc2[nH]nc(-c3ccccc3)c2C1] | 349.1790 | 349.0 |
| 3-33 | 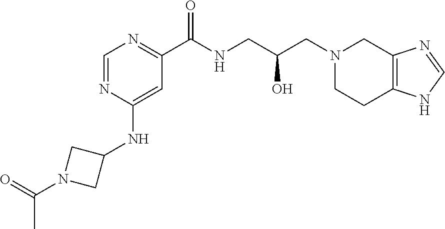 [SMILES: CC(=O)N1CC(Nc2cc(C(=O)NC[C@H](O)CN3CCc4[nH]cnc4C3)ncn2)C1] | 414.2128 | 415.3 |
| 3-34 | 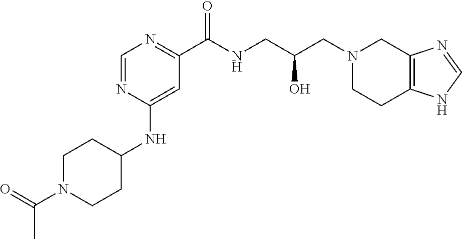 [SMILES: CC(=O)N1CCC(Nc2cc(C(=O)NC[C@H](O)CN3CCc4[nH]cnc4C3)ncn2)CC1] | 442.2441 | 443.3 |
| 3-35 | 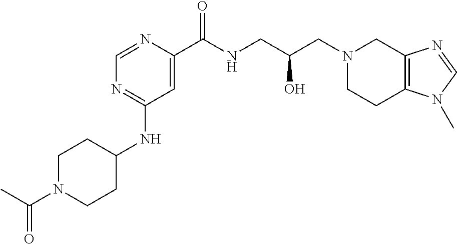 [SMILES: CC(=O)N1CCC(Nc2cc(C(=O)NC[C@H](O)CN3CCc4c(ncn4C)C3)ncn2)CC1] | 456.2597 | 457.3 |
| 3-36 | 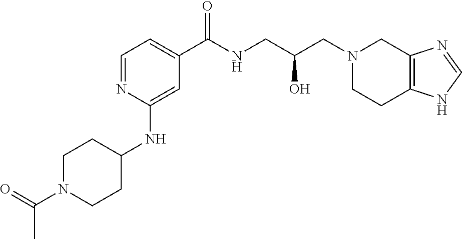 [SMILES: CC(=O)N1CCC(Nc2cc(C(=O)NC[C@H](O)CN3CCc4[nH]cnc4C3)ccn2)CC1] | 441.2488 | 442.3 |
| 3-37 | 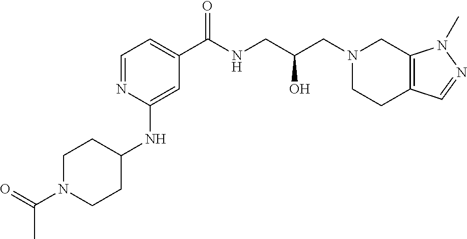 [SMILES: CC(=O)N1CCC(Nc2cc(C(=O)NC[C@H](O)CN3CCc4cnn(C)c4C3)ccn2)CC1] | 455.2645 | 456.3 |
| 3-38 | 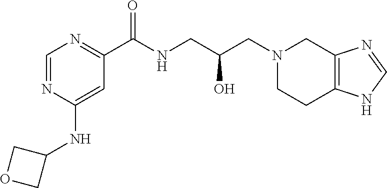 [SMILES: O=C(NC[C@H](O)CN1CCc2[nH]cnc2C1)c1cc(NC2COC2)ncn1] | 373.1862 | 374.2 |
| 3-39 | 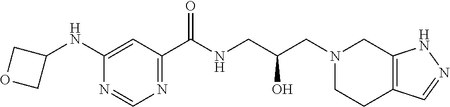 [SMILES: O=C(NC[C@H](O)CN1CCc2cn[nH]c2C1)c1cc(NC2COC2)ncn1] | 373.1862 | 373.41 |
| 3-40 | 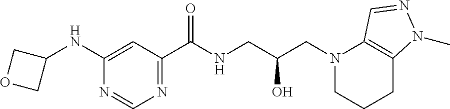 [SMILES: Cn1ncc2c1CCCN2C[C@@H](O)CNC(=O)c1cc(NC2COC2)ncn1] | 387.2019 | 388.2 |
| 3-41 | 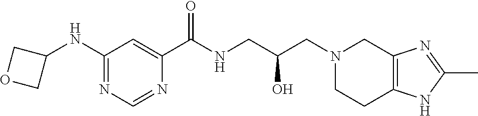 [SMILES: Cc1nc2c([nH]1)CCN(C[C@@H](O)CNC(=O)c1cc(NC3COC3)ncn1)C2] | 387.2019 | 388.2 |
| 3-42 | 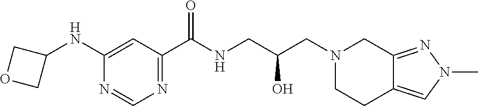 [SMILES: Cn1cc2c(n1)CN(C[C@@H](O)CNC(=O)c1cc(NC3COC3)ncn1)CC2] | 387.2019 | 388.2 |
| 3-43 | 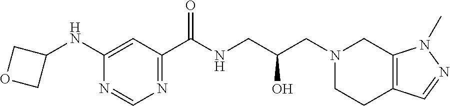 [SMILES: Cn1ncc2c1CN(C[C@@H](O)CNC(=O)c1cc(NC3COC3)ncn1)CC2] | 387.2019 | 388.2 |
| 3-44 | 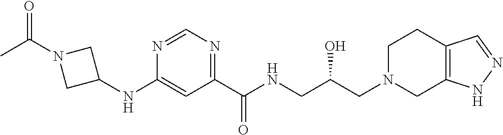 [SMILES: CC(=O)N1CC(Nc2cc(C(=O)NC[C@H](O)CN3CCc4cn[nH]c4C3)ncn2)C1] | 414.2128 | 415.2 |
| 3-45 | 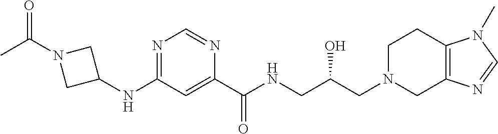 [SMILES: CC(=O)N1CC(Nc2cc(C(=O)NC[C@H](O)CN3CCc4c(ncn4C)C3)ncn2)C1] | 428.2284 | 429.3 |
| 3-46 | 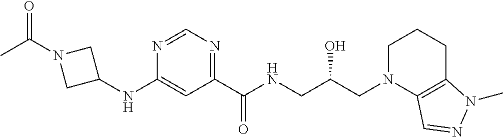 [SMILES: CC(=O)N1CC(Nc2cc(C(=O)NC[C@H](O)CN3CCCc4c3cnn4C)ncn2)C1] | 428.2284 | 429.3 |
| 3-47 | 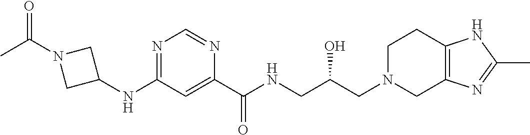 [SMILES: CC(=O)N1CC(Nc2cc(C(=O)NC[C@H](O)CN3CCc4[nH]c(C)nc4C3)ncn2)C1] | 428.2284 | 429.1 |
| 3-48 | 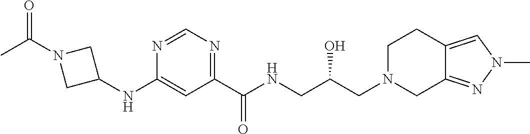 [SMILES: CC(=O)N1CC(Nc2cc(C(=O)NC[C@H](O)CN3CCc4cn(C)nc4C3)ncn2)C1] | 428.2284 | 429.1 |
| 3-49 | 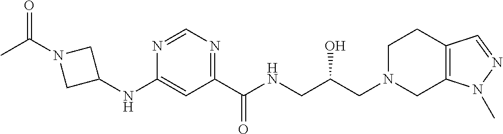 [SMILES: CC(=O)N1CC(Nc2cc(C(=O)NC[C@H](O)CN3CCc4cnn(C)c4C3)ncn2)C1] | 428.2284 | 429.3 |
| 3-50 | 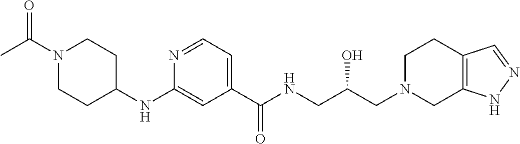 [SMILES: CC(=O)N1CCC(Nc2cc(C(=O)NC[C@H](O)CN3CCc4cn[nH]c4C3)ccn2)CC1] | 441.2488 | 442.3 |
| 3-51 | 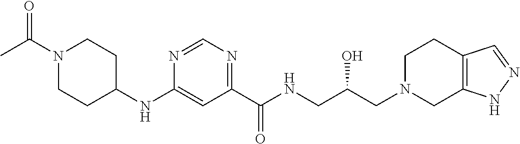 [SMILES: CC(=O)N1CCC(Nc2cc(C(=O)NC[C@H](O)CN3CCc4cn[nH]c4C3)ncn2)CC1] | 442.2441 | 443.3 |
| 3-52 | 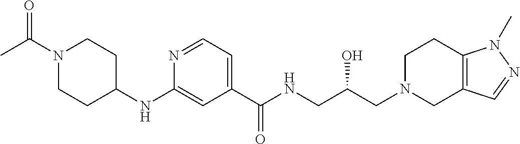 [SMILES: CC(=O)N1CCC(Nc2cc(C(=O)NC[C@H](O)CN3CCc4c(cnn4C)C3)ccn2)CC1] | 455.2645 | 456.3 |
| 3-53 | 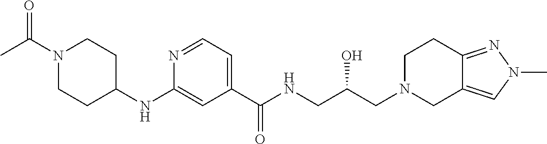 [SMILES: CC(=O)N1CCC(Nc2cc(C(=O)NC[C@H](O)CN3CCc4nn(C)cc4C3)ccn2)CC1] | 455.2645 | 456.3 |
| 3-54 | 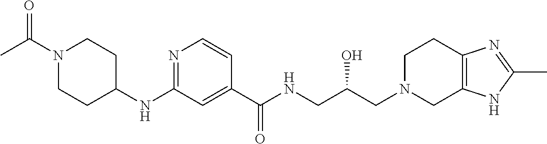 [SMILES: CC(=O)N1CCC(Nc2cc(C(=O)NC[C@H](O)CN3CCc4nc(C)[nH]c4C3)ccn2)CC1] | 455.2645 | 456.3 |
| 3-55 | 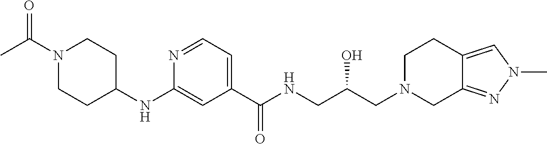 [SMILES: CC(=O)N1CCC(Nc2cc(C(=O)NC[C@H](O)CN3CCc4cn(C)nc4C3)ccn2)CC1] | 455.2645 | 456.3 |
| 3-56 | 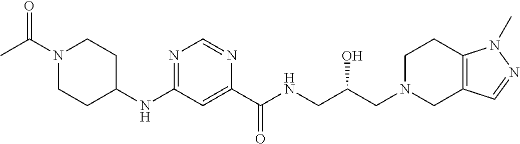 [SMILES: CC(=O)N1CCC(Nc2cc(C(=O)NC[C@H](O)CN3CCc4c(cnn4C)C3)ncn2)CC1] | 456.2597 | 457.2 |
| 3-57 | 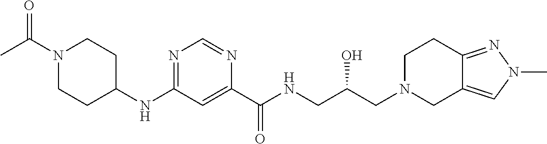 [SMILES: CC(=O)N1CCC(Nc2cc(C(=O)NC[C@H](O)CN3CCc4nn(C)cc4C3)ncn2)CC1] | 456.2597 | 457.1 |
| 3-58 | 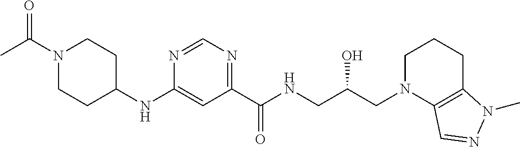 [SMILES: CC(=O)N1CCC(Nc2cc(C(=O)NC[C@H](O)CN3CCCc4c3cnn4C)ncn2)CC1] | 456.2597 | 457.1 |
| 3-59 | 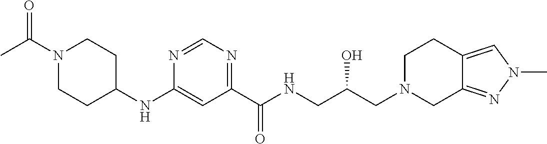 [SMILES: CC(=O)N1CCC(Nc2cc(C(=O)NC[C@H](O)CN3CCc4cn(C)nc4C3)ncn2)CC1] | 456.2597 | 457.3 |
| 3-60 | 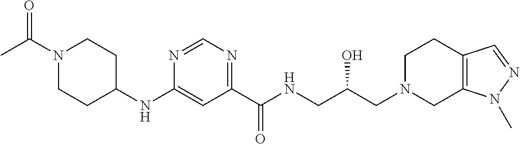 [SMILES: CC(=O)N1CCC(Nc2cc(C(=O)NC[C@H](O)CN3CCc4cnn(C)c4C3)ncn2)CC1] | 456.2597 | 457.3 |

In certain embodiments, a provided compound is a compound listed in Table 1D, or a pharmaceutically acceptable salt thereof.

TABLE 1D
Exemplary Compounds

| # | Compound | Exact Mass | LCMS |
|---|---|---|---|
| 4-1 | 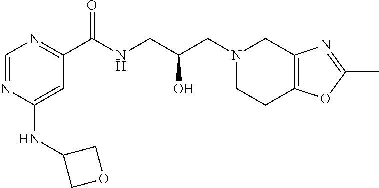 [SMILES: Cc1nc2c(o1)CCN(C[C@@H](O)CNC(=O)c1cc(NC3COC3)ncn1)C2] | 388.1859 | 389.2 |
| 4-2 | 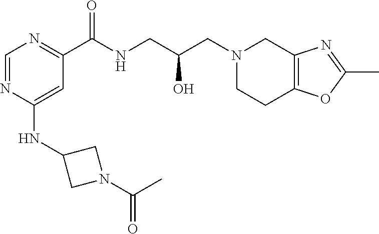 [SMILES: CC(=O)N1CC(Nc2cc(C(=O)NC[C@H](O)CN3CCc4oc(C)nc4C3)ncn2)C1] | 429.2125 | 430.2 |
| 4-3 | 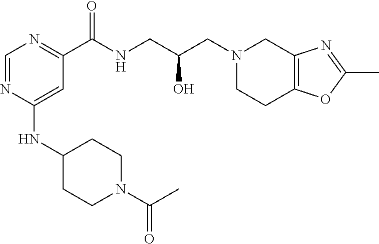 [SMILES: CC(=O)N1CCC(Nc2cc(C(=O)NC[C@H](O)CN3CCc4oc(C)nc4C3)ncn2)CC1] | 457.2438 | 458.3 |
| 4-4 | 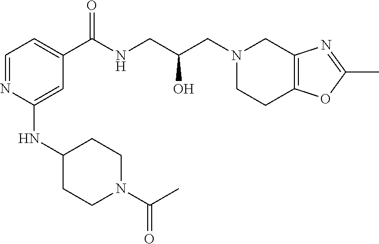 [SMILES: CC(=O)N1CCC(Nc2cc(C(=O)NC[C@H](O)CN3CCc4oc(C)nc4C3)ccn2)CC1] | 456.2485 | 457.3 |

In certain embodiments, a provided compound is a compound listed in Table 1E, or a pharmaceutically acceptable salt thereof.

TABLE 1E
Exemplary compounds

| # | Compound | Exact Mass | LCMS |
|---|---|---|---|
| 5-1 | 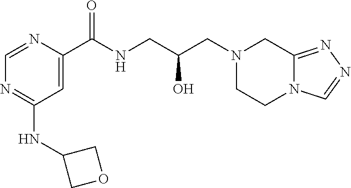 [SMILES: O=C(NC[C@H](O)CN1CCn2cnnc2C1)c1cc(NC2COC2)ncn1] | 374.1815 | — |
| 5-2 | 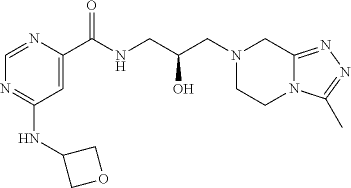 [SMILES: Cc1nnc2n1CCN(C[C@@H](O)CNC(=O)c1cc(NC3COC3)ncn1)C2] | 388.1971 | — |
| 5-3 | 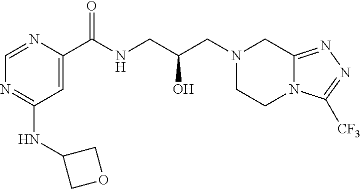 [SMILES: O=C(NC[C@H](O)CN1CCn2c(nnc2C(F)(F)F)C1)c1cc(NC2COC2)ncn1] | 442.1689 | — |
| 5-4 | 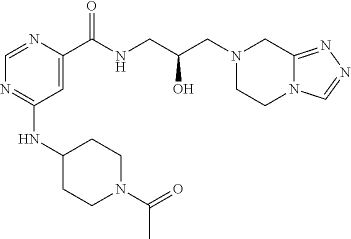 [SMILES: CC(=O)N1CCC(Nc2cc(C(=O)NC[C@H](O)CN3CCn4cnnc4C3)ncn2)CC1] | 443.2393 | — |
| 5-5 | 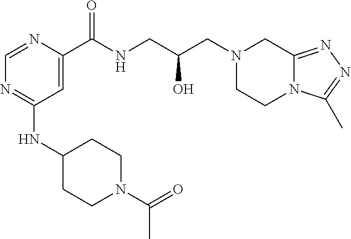 [SMILES: CC(=O)N1CCC(Nc2cc(C(=O)NC[C@H](O)CN3CCn4c(C)nnc4C3)ncn2)CC1] | 457.2550 | — |
| 5-6 | 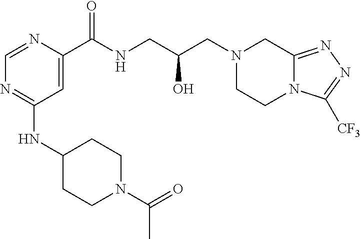 [SMILES: CC(=O)N1CCC(Nc2cc(C(=O)NC[C@H](O)CN3CCn4c(nnc4C(F)(F)F)C3)ncn2)CC1] | 511.2267 | — |
| 5-7 | 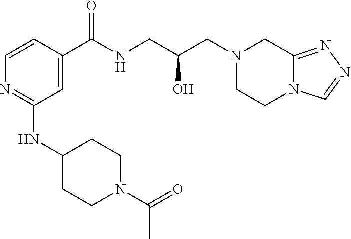 [SMILES: CC(=O)N1CCC(Nc2cc(C(=O)NC[C@H](O)CN3CCn4cnnc4C3)ccn2)CC1] | 442.2441 | — |
| 5-8 | 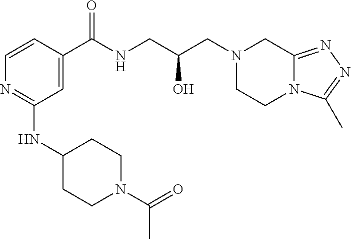 [SMILES: CC(=O)N1CCC(Nc2cc(C(=O)NC[C@H](O)CN3CCn4c(C)nnc4C3)ccn2)CC1] | 456.2597 | — |
| 5-9 | 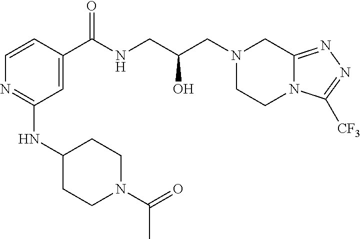 [SMILES: CC(=O)N1CCC(Nc2cc(C(=O)NC[C@H](O)CN3CCn4c(nnc4C(F)(F)F)C3)ccn2)CC1] | 510.2315 | — |
| 5-10 | 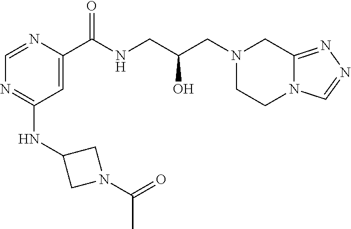 [SMILES: CC(=O)N1CC(Nc2cc(C(=O)NC[C@H](O)CN3CCn4cnnc4C3)ncn2)C1] | 415.2080 | — |
| 5-11 | 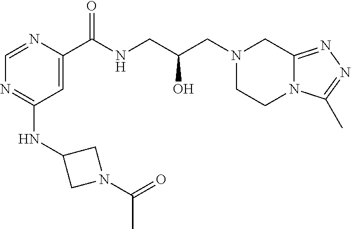 [SMILES: CC(=O)N1CC(Nc2cc(C(=O)NC[C@H](O)CN3CCn4c(C)nnc4C3)ncn2)C1] | 429.2237 | — |
| 5-12 | 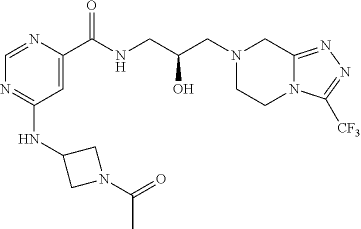 [SMILES: CC(=O)N1CC(Nc2cc(C(=O)NC[C@H](O)CN3CCn4c(nnc4C(F)(F)F)C3)ncn2)C1] | 483.1954 | — |
| 5-13 | 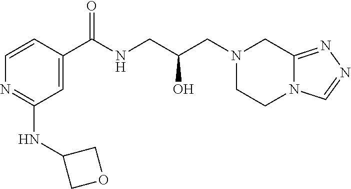 [SMILES: O=C(NC[C@H](O)CN1CCn2cnnc2C1)c1ccnc(NC2COC2)c1] | 373.1862 | — |
| 5-14 | 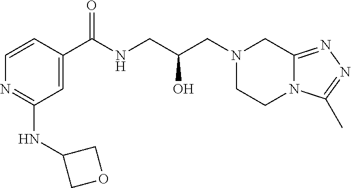 [SMILES: Cc1nnc2n1CCN(C[C@@H](O)CNC(=O)c1ccnc(NC3COC3)c1)C2] | 387.2019 | — |
| 5-15 | 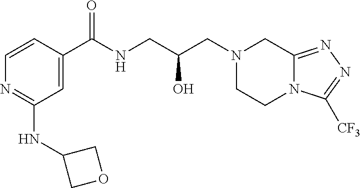 [SMILES: O=C(NC[C@H](O)CN1CCn2c(nnc2C(F)(F)F)C1)c1ccnc(NC2COC2)c1] | 441.1736 | — |
| 5-16 | 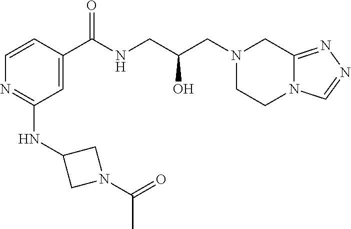 [SMILES: CC(=O)N1CC(Nc2cc(C(=O)NC[C@H](O)CN3CCn4cnnc4C3)ccn2)C1] | 414.2128 | — |
| 5-17 | 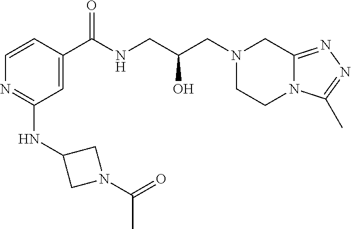 [SMILES: CC(=O)N1CC(Nc2cc(C(=O)NC[C@H](O)CN3CCn4c(C)nnc4C3)ccn2)C1] | 428.2284 | — |
| 5-18 | 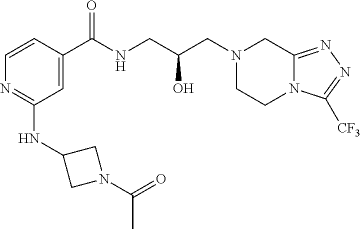 [SMILES: CC(=O)N1CC(Nc2cc(C(=O)NC[C@H](O)CN3CCn4c(nnc4C(F)(F)F)C3)ccn2)C1] | 482.2002 | — |

In certain embodiments, a provided compound is not one of the following compounds:

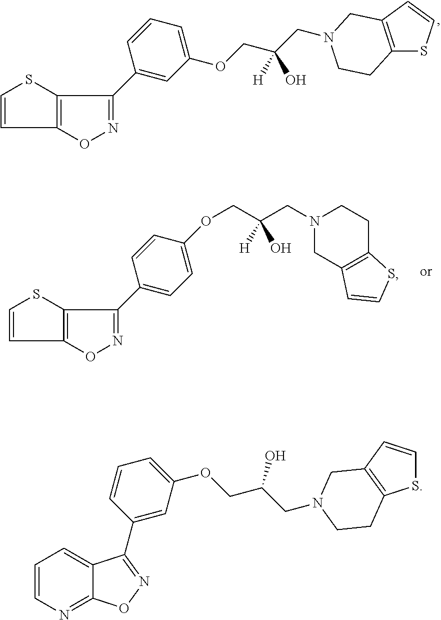 [SMILES: O[C@@H](COc1cccc(-c2noc3ccsc23)c1)CN1CCc2sccc2C1 ; O[C@@H](COc1ccc(-c2noc3ccsc23)cc1)CN1CCc2sccc2C1 ; O[C@@H](COc1cccc(-c2noc3ncccc23)c1)CN1CCc2sccc2C1]

In certain embodiments, a provided compound inhibits PRMT5. In certain embodiments, a provided compound inhibits wild-type PRMT5. In certain embodiments, a provided compound inhibits a mutant PRMT5. In certain embodiments, a provided compound inhibits PRMT5, e.g., as measured in an assay described herein. In certain embodiments, the PRMT5 is from a human. In certain embodiments, a provided compound inhibits PRMT5 at an IC₅₀ less than or equal to 10 μM. In certain embodiments, a provided compound inhibits PRMT5 at an IC₅₀ less than or equal to 1 μM. In certain embodiments, a provided compound inhibits PRMT5 at an IC₅₀ less than or equal to 0.1 μM. In certain embodiments, a provided compound inhibits PRMT5 in a cell at an EC₅₀ less than or equal to 10 μM. In certain embodiments, a provided compound inhibits PRMT5 in a cell at an EC₅₀ less than or equal to 1 μM. In certain embodiments, a provided compound inhibits PRMT5 in a cell at an EC₅₀ less than or equal to 0.1 μM. In certain embodiments, a provided compound inhibits cell proliferation at an EC₅₀ less than or equal to 10 μM. In certain embodiments, a provided compound inhibits cell proliferation at an EC₅₀ less than or equal to 1 μM. In certain embodiments, a provided compound inhibits cell proliferation at an EC₅₀ less than or equal to 0.1 μM. In some embodiments, a provided compound is selective for PRMT5 over other methyltransferases. In certain embodiments, a provided compound is at least about 10-fold selective, at least about 20-fold selective, at least about 30-fold selective, at least about 40-fold selective, at least about 50-fold selective, at least about 60-fold selective, at least about 70-fold selective, at least about 80-fold selective, at least about 90-fold selective, or at least about 100-fold selective for PRMT5 relative to one or more other methyltransferases.

It will be understood by one of ordinary skill in the art that the PRMT5 can be wild-type PRMT5, or any mutant or variant of PRMT5.

In certain embodiments, the PRMT5 is isoform A (GenBank accession no. NP006100) (SEQ ID NO.:1):

MAAMAVGGAG GSRVSSGRDL NCVPEIADTL GAVAKQGFDF
LCMPVFHPRF KREFIQEPAK NRPGPQTRSD LLLSGRDWNT
LIVGKLSPWI RPDSKVEKIR RNSEAAMLQE LNFGAYLGLP
AFLLPLNQED NTNLARVLTN HIHTGHHSSM FWMRVPLVAP
EDLRDDIIEN APTTHTEEYS GEEKTWMWWH NFRTLCDYSK
RIAVALEIGA DLPSNHVIDR WLGEPIKAAI LPTSIFLTNK
KGFPVLSKMH QRLIFRLLKL EVQFIITGTN HHSEKEFCSY
LQYLEYLSQN RPPPNAYELF AKGYEDYLQS PLQPLMDNLE
SQTYEVFEKD PIKYSQYQQA IYKCLLDRVP EEEKDTNVQV
LMVLGAGRGP LVNASLRAAK QADRRIKLYA VEKNPNAVVT
LENWQFEEWG SQVTVVSSDM REWVAPEKAD IIVSELLGSF
ADNELSPECL DGAQHFLKDD GVSIPGEYTS FLAPISSSKL
YNEVRACREK DRDPEAQFEM PYVVRLHNFH QLSAPQPCFT
FSHPNRDPMI DNNRYCTLEF PVEVNTVLHG FAGYFETVLY
QDITLSIRPE THSPGMFSWF PILFPIKQPI TVREGQTICV
RFWRCSNSKK VWYEWAVTAP VCSAIHNPTG RSYTIGL

In certain embodiments, the PRMT5 is isoform B (GenBank accession no. NP001034708) (SEQ ID NO.:2)

MRGPNSGTEK GRLVIPEKQG FDFLCMPVFH PRFKREFIQE
PAKNRPGPQT RSDLLLSGRD WNTLIVGKLS PWIRPDSKVE
KIRRNSEAAM LQELNFGAYL GLPAFLLPLN QEDNTNLARV
LTNHIHTGHH SSMFWMRVPL VAPEDLRDDI IENAPTTHTE
EYSGEEKTWM WWHNFRTLCD YSKRIAVALE IGADLPSNHV
IDRWLGEPIK AAILPTSIFL TNKKGFPVLS KMHQRLIFRL
LKLEVQFIIT GTNHHSEKEF CSYLQYLEYL SQNRPPPNAY
ELFAKGYEDY LQSPLQPLMD NLESQTYEVF EKDPIKYSQY
QQAIYKCLLD RVPEEEKDTN VQVLMVLGAG RGPLVNASLR
AAKQADRRIK LYAVEKNPNA VVTLENWQFE EWGSQVTVVS
SDMREWVAPE KADIIVSELL GSFADNELSP ECLDGAQHFL
KDDGVSIPGE YTSFLAPISS SKLYNEVRAC REKDRDPEAQ
FEMPYVVRLH NFHQLSAPQP CFTFSHPNRD PMIDNNRYCT
LEFPVEVNTV LHGFAGYFET VLYQDITLSI RPETHSPGMF
SWFPILFPIK QPITVREGQT ICVRFWRCSN SKKVWYEWAV
TAPVCSAIHN PTGRSYTIGL

In certain embodiments, the PRMT5 is transcript variant 1 (GenBank accession no. NM_006109).

The present disclosure provides pharmaceutical compositions comprising a compound described herein, e.g., a compound of Formula (A), or a pharmaceutically acceptable salt thereof, as described herein, and optionally a pharmaceutically acceptable excipient. It will be understood by one of ordinary skill in the art that the compounds described herein, or salts thereof, may be present in various forms, such as hydrates, solvates, or polymorphs. In certain embodiments, a provided composition comprises two or more compounds described herein. In certain embodiments, a compound described herein, or a pharmaceutically acceptable salt thereof, is provided in an effective amount in the pharmaceutical composition. In certain embodiments, the effective amount is a therapeutically effective amount. In certain embodiments, the effective amount is an amount effective for inhibiting PRMT5. In certain embodiments, the effective amount is an amount effective for treating a PRMT5-mediated disorder. In certain embodiments, the effective amount is a prophylactically effective amount. In certain embodiments, the effective amount is an amount effective to prevent a PRMT5-mediated disorder.

Pharmaceutically acceptable excipients include any and all solvents, diluents, or other liquid vehicles, dispersions, suspension aids, surface active agents, isotonic agents, thickening or emulsifying agents, preservatives, solid binders, lubricants, and the like, as suited to the particular dosage form desired. General considerations in formulation and/or manufacture of pharmaceutical compositions agents can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Pharmaceutical compositions described herein can be prepared by any method known in the art of pharmacology. In general, such preparatory methods include the steps of bringing a compound described herein (the “active ingredient”) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition of the present disclosure will vary, depending upon the identity, size, and/or condition of the subject treated and further depending upon the route by which the composition is to be administered. By way of example, the composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween 20), polyoxyethylene sorbitan (Tween 60), polyoxyethylene sorbitan monooleate (Tween 80), sorbitan monopalmitate (Span 40), sorbitan monostearate (Span 60], sorbitan tristearate (Span 65), glyceryl monooleate, sorbitan monooleate (Span 80)), polyoxyethylene esters (e.g., polyoxyethylene monostearate (Myrj 45), polyoxyethylene hydrogenated castor oil, polyethoxylated castor oil, polyoxymethylene stearate, and Solutol), sucrose fatty acid esters, polyethylene glycol fatty acid esters (e.g., Cremophor™), polyoxyethylene ethers, (e.g., polyoxyethylene lauryl ether (Brij 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F68, Poloxamer 188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol. Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the compounds described herein are mixed with solubilizing agents such as Cremophor™, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension or emulsion in a nontoxic parenterally acceptable diluent or solvent, for example, as a solution in 1,3-butanediol. Among the acceptable vehicles and solvents that can be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Compositions for rectal or vaginal administration are typically suppositories which can be prepared by mixing the compounds described herein with suitable non-irritating excipients or carriers such as cocoa butter, polyethylene glycol or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may comprise buffering agents.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type can be employed as fillers in soft and hard-filled gelatin capsules using such excipients as lactose or milk sugar as well as high molecular weight polyethylene glycols and the like.

The active ingredient can be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets, and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes.

Dosage forms for topical and/or transdermal administration of a provided compound may include ointments, pastes, creams, lotions, gels, powders, solutions, sprays, inhalants and/or patches. Generally, the active ingredient is admixed under sterile conditions with a pharmaceutically acceptable carrier and/or any desired preservatives and/or buffers as can be required. Additionally, the present disclosure encompasses the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices such as those described in U.S. Pat. Nos. 4,886,499; 5,190,521; 5,328,483; 5,527,288; 4,270,537; 5,015,235; 5,141,496; and 5,417,662. Intradermal compositions can be administered by devices which limit the effective penetration length of a needle into the skin, such as those described in PCT publication WO 99/34850 and functional equivalents thereof. Jet injection devices which deliver liquid vaccines to the dermis via a liquid jet injector and/or via a needle which pierces the stratum corneum and produces a jet which reaches the dermis are suitable. Jet injection devices are described, for example, in U.S. Pat. Nos. 5,480,381; 5,599,302; 5,334,144; 5,993,412; 5,649,912; 5,569,189; 5,704,911; 5,383,851; 5,893,397; 5,466,220; 5,339,163; 5,312,335; 5,503,627; 5,064,413; 5,520,639; 4,596,556; 4,790,824; 4,941,880; 4,940,460; and PCT publications WO 97/37705 and WO 97/13537. Ballistic powder/particle delivery devices which use compressed gas to accelerate vaccine in powder form through the outer layers of the skin to the dermis are suitable. Alternatively or additionally, conventional syringes can be used in the classical mantoux method of intradermal administration.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions. Topically-administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A provided pharmaceutical composition can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers or from about 1 to about 6 nanometers. Such compositions are conveniently in the form of dry powders for administration using a device comprising a dry powder reservoir to which a stream of propellant can be directed to disperse the powder and/or using a self propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) and as much as 100% (w/w) of the active ingredient, and may comprise one or more of the additional ingredients described herein. A provided pharmaceutical composition can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A provided pharmaceutical composition can be prepared, packaged, and/or sold in a formulation for ophthalmic administration. Such formulations may, for example, be in the form of eye drops including, for example, a 0.1/1.0% (w/w) solution and/or suspension of the active ingredient in an aqueous or oily liquid carrier. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form for ease of administration and uniformity of dosage. It will be understood, however, that the total daily usage of provided compositions will be decided by the attending physician within the scope of sound medical judgment. The specific therapeutically effective dose level for any particular subject or organism will depend upon a variety of factors including the disease, disorder, or condition being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general the most appropriate route of administration will depend upon a variety of factors including the nature of the agent (e.g., its stability in the environment of the gastrointestinal tract), and/or the condition of the subject (e.g., whether the subject is able to tolerate oral administration).

The exact amount of a compound required to achieve an effective amount will vary from subject to subject, depending, for example, on species, age, and general condition of a subject, severity of the side effects or disorder, identity of the particular compound(s), mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, a compound described herein may be administered at dosage levels sufficient to deliver from about 0.001 mg/kg to about 1000 mg/kg, from about 0.01 mg/kg to about mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic effect.

In some embodiments, a compound described herein is administered one or more times per day, for multiple days. In some embodiments, the dosing regimen is continued for days, weeks, months, or years.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

It will be also appreciated that a compound or composition, as described herein, can be administered in combination with one or more additional therapeutically active agents. In certain embodiments, a compound or composition provided herein is administered in combination with one or more additional therapeutically active agents that improve its bioavailability, reduce and/or modify its metabolism, inhibit its excretion, and/or modify its distribution within the body. It will also be appreciated that the therapy employed may achieve a desired effect for the same disorder, and/or it may achieve different effects.

The compound or composition can be administered concurrently with, prior to, or subsequent to, one or more additional therapeutically active agents. In certain embodiments, the additional therapeutically active agent is a compound of Formula (A). In certain embodiments, the additional therapeutically active agent is not a compound of Formula (A). In general, each agent will be administered at a dose and/or on a time schedule determined for that agent. In will further be appreciated that the additional therapeutically active agent utilized in this combination can be administered together in a single composition or administered separately in different compositions. The particular combination to employ in a regimen will take into account compatibility of a provided compound with the additional therapeutically active agent and/or the desired therapeutic effect to be achieved. In general, it is expected that additional therapeutically active agents utilized in combination be utilized at levels that do not exceed the levels at which they are utilized individually. In some embodiments, the levels utilized in combination will be lower than those utilized individually.

Exemplary additional therapeutically active agents include, but are not limited to, small organic molecules such as drug compounds (e.g., compounds approved by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells.

Also encompassed by the present disclosure are kits (e.g., pharmaceutical packs). The kits provided may comprise a provided pharmaceutical composition or compound and a container (e.g., a vial, ampule, bottle, syringe, and/or dispenser package, or other suitable container). In some embodiments, provided kits may optionally further include a second container comprising a pharmaceutical excipient for dilution or suspension of a provided pharmaceutical composition or compound. In some embodiments, a provided pharmaceutical composition or compound provided in the container and the second container are combined to form one unit dosage form. In some embodiments, a provided kits further includes instructions for use.

Compounds and compositions described herein are generally useful for the inhibition of PRMT5. In some embodiments, methods of treating PRMT5-mediated disorder in a subject are provided which comprise administering an effective amount of a compound described herein (e.g., a compound of Formula (A)), or a pharmaceutically acceptable salt thereof), to a subject in need of treatment. In certain embodiments, the effective amount is a therapeutically effective amount. In certain embodiments, the effective amount is a prophylactically effective amount. In certain embodiments, the subject is suffering from a PRMT5-mediated disorder. In certain embodiments, the subject is susceptible to a PRMT5-mediated disorder.

As used herein, the term “PRMT5-mediated disorder” means any disease, disorder, or other pathological condition in which PRMT5 is known to play a role. Accordingly, in some embodiments, the present disclosure relates to treating or lessening the severity of one or more diseases in which PRMT5 is known to play a role.

In some embodiments, the present disclosure provides a method of inhibiting PRMT5 comprising contacting PRMT5 with an effective amount of a compound described herein (e.g., a compound of Formula (A)) or a pharmaceutically acceptable salt thereof. The PRMT5 may be purified or crude, and may be present in a cell, tissue, or subject. Thus, such methods encompass both inhibition of in vitro and in vivo PRMT5 activity. In certain embodiments, the method is an in vitro method, e.g., such as an assay method. It will be understood by one of ordinary skill in the art that inhibition of PRMT5 does not necessarily require that all of the PRMT5 be occupied by an inhibitor at once. Exemplary levels of inhibition of PRMT5 include at least 10% inhibition, about 10% to about 25% inhibition, about 25% to about 50% inhibition, about 50% to about 75% inhibition, at least 50% inhibition, at least 75% inhibition, about 80% inhibition, about 90% inhibition, and greater than 90% inhibition.

In some embodiments, provided is a method of inhibiting PRMT5 activity in a subject in need thereof comprising administering to the subject an effective amount of a compound described herein (e.g., a compound of Formula (A)), or a pharmaceutically acceptable salt thereof, or a pharmaceutical composition thereof.

In certain embodiments, provided is a method of altering gene expression in a cell which comprises contacting a cell with an effective amount of a compound of Formula (A), or a pharmaceutically acceptable salt thereof. In certain embodiments, the cell in culture in vitro. In certain embodiments, the cell is in an animal, e.g., a human. In certain embodiments, the cell is in a subject in need of treatment.

In certain embodiments, provided is a method of altering transcription in a cell which comprises contacting a cell with an effective amount of a compound of Formula (A), or a pharmaceutically acceptable salt thereof. In certain embodiments, the cell in culture in vitro. In certain embodiments, the cell is in an animal, e.g., a human. In certain embodiments, the cell is in a subject in need of treatment.

In certain embodiments, a method is provided of selecting a therapy for a subject having a disease associated with PRMT5-mediated disorder or mutation comprising the steps of determining the presence of PRMT5-mediated disorder or gene mutation in the PRMT5 gene or and selecting, based on the presence of PRMT5-mediated disorder a gene mutation in the PRMT5 gene a therapy that includes the administration of a provided compound. In certain embodiments, the disease is cancer.

In certain embodiments, a method of treatment is provided for a subject in need thereof comprising the steps of determining the presence of PRMT5-mediated disorder or a gene mutation in the PRMT5 gene and treating the subject in need thereof, based on the presence of a PRMT5-mediated disorder or gene mutation in the PRMT5 gene with a therapy that includes the administration of a provided compound. In certain embodiments, the subject is a cancer patient.

In some embodiments, a provided compound is useful in treating a proliferative disorder, such as cancer, a benign neoplasm, an autoimmune disease, or an inflammatory disease. For example, while not being bound to any particular mechanism, PRMT5 has been shown to be involved in cyclin D1 dysregulated cancers. Increased PRMT5 activity mediates key events associated with cyclin D1-dependent neoplastic growth including CUL4 repression, CDT1 overexpression, and DNA re-replication. Further, human cancers harboring mutations in Fbx4, the cyclin D1 E3 ligase, exhibit nuclear cyclin D1 accumulation and increased PRMT5 activity. See, e.g., Aggarwal et al., Cancer Cell. (2010) 18(4):329-40. Additionally, PRMT5 has also been implicated in accelerating cell cycle progression through G1 phase and modulating regulators of G1; for example, PRMT5 may upregulate cyclin-dependent kinase (CDK) 4, CDK6, and cyclins D1, D2 and E1. Moreover, PRMT5 may activate phosphoinositide 3-kinase (PI3K)/AKT signaling. See, e.g., Wei et al., Cancer Sci. (2012) 103(9):1640-50. PRMT5 has been reported to play a role in apoptosis through methylation of E2F-1. See, e.g., Cho et al., EMBO J. (2012) 31:1785-1797; Zheng et al., Mol. Cell. (2013) 52:37-51. PRMT5 has been reported to be an essential regulator of splicing and affect the alternative splicing of ‘sensor’ mRNAs that can then lead to defects in downstream events such as apoptosis. See, e.g., Bezzi et al., Genes Dev. (2013) 27:1903-1916. PRMT5 has been reported to play a role in the RAS-ERK pathway. See, e.g., Andrew-Perez et al., Sci Signal. (2011) Sep. 13; 4(190)ra58 doi: 10.1126/scisignal.2001936. PRMT5 has been reported to affect C/EBPb target genes through interaction with the Mediator complex and hence affect cellular differentiation and inflammatory response. See, e.g., Tsutsui et al., J. Biol. Chem. (2013) 288:20955-20965. PRMT5 has been shown to methylate HOXA9 essential for ELAM expression during the EC inflammatory response. See, e.g., Bandyopadhyay et al., Mol. Cell. Biol. (2012) 32:1202-1203. Thus in some embodiments, the inhibition of PRMT5 by a provided compound is useful in treating the following non-limiting list of cancers: breast cancer, esophageal cancer, bladder cancer, lung cancer, hematopoietic cancer, lymphoma, medulloblastoma, rectum adenocarcinoma, colon adenocarcinoma, gastric cancer, pancreatic cancer, liver cancer, adenoid cystic carcinoma, lung adenocarcinoma, head and neck squamous cell carcinoma, brain tumors, hepatocellular carcinoma, renal cell carcinoma, melanoma, oligodendroglioma, ovarian clear cell carcinoma, and ovarian serous cystadenocarcinoma. See, e.g., Pal et al., EMBO J. (2007) 26:3558-3569 (mantle cell lymphoma); Wang et al., Mol. Cell Biol. (2008) 28:6262-77 (chronic lymphocytic leukemia (CLL)); and Tae et al., Nucleic Acids Res. (2011) 39:5424-5438.

In some embodiments, the inhibition of PRMT5 by a provided compound is useful in treating prostate cancer and lung cancer, in which PRMT5 has been shown to play a role. See, e.g., Gu et al., PLoS One 2012; 7(8):e44033; Gu et al., Biochem. J. (2012) 446:235-241. In some embodiments, a provided compound is useful to delay the onset of, slow the progression of, or ameliorate the symptoms of cancer. In some embodiments, a provided compound is administered in combination with other compounds, drugs, or therapeutics to treat cancer.

In some embodiments, compounds described herein are useful for treating a cancer including, but not limited to, acoustic neuroma, adenocarcinoma, adrenal gland cancer, anal cancer, angiosarcoma (e.g., lymphangiosarcoma, lymphangioendotheliosarcoma, hemangiosarcoma), appendix cancer, benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma; medulloblastoma), bronchus cancer, carcinoid tumor, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer (e.g., uterine cancer, uterine sarcoma), esophageal cancer (e.g., adenocarcinoma of the esophagus, Barrett's adenocarinoma), Ewing sarcoma, eye cancer (e.g., intraocular melanoma, retinoblastoma), familiar hypereosinophilia, gall bladder cancer, gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC), throat cancer (e.g., laryngeal cancer, pharyngeal cancer, nasopharyngeal cancer, oropharyngeal cancer)), hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., “Waldenstrim's macroglobulinemia”), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma); a mixture of one or more leukemia/lymphoma as described above; and multiple myeloma (MM)), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leiomyosarcoma (LMS), mastocytosis (e.g., systemic mastocytosis), myelodysplastic syndrome (MDS), mesothelioma, myeloproliferative disorder (MPD) (e.g., polycythemia Vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), papillary adenocarcinoma, pancreatic cancer (e.g., pancreatic andenocarcinoma, intraductal papillary mucinous neoplasm (IPMN), Islet cell tumors), penile cancer (e.g., Paget's disease of the penis and scrotum), pinealoma, primitive neuroectodermal tumor (PNT), prostate cancer (e.g., prostate adenocarcinoma), rectal cancer, rhabdomyosarcoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), urethral cancer, vaginal cancer, and vulvar cancer (e.g., Paget's disease of the vulva).

In some embodiments, a provided compound is useful in treating a metabolic disorder, such as diabetes or obesity. For example, while not being bound to any particular mechanism, a role for PRMT5 has been recognized in adipogenesis. Inhibition of PRMT5 expression in multiple cell culture models for adipogenesis prevented the activation of adipogenic genes, while overexpression of PRMT5 enhanced adipogenic gene expression and differentiation. See, e.g., LeBlanc et al., Mol Endocrinol. (2012) 26:583-597. Additionally, it has been shown that adipogenesis plays a pivotal role in the etiology and progression of diabetes and obesity. See, e.g., Camp et al., Trends Mol Med. (2002) 8:442-447. Thus in some embodiments, the inhibition of PRMT5 by a provided compound is useful in treating diabetes and/or obesity.

In some embodiments, a provided compound is useful to delay the onset of, slow the progression of, or ameliorate the symptoms of, diabetes. In some embodiments, the diabetes is Type 1 diabetes. In some embodiments, the diabetes is Type 2 diabetes. In some embodiments, a provided compound is useful to delay the onset of, slow the progression of, or ameliorate the symptoms of, obesity. In some embodiments, a provided compound is useful to help a subject lose weight. In some embodiments, a provided compound could be used in combination with other compounds, drugs, or therapeutics, such as metformin and insulin, to treat diabetes and/or obesity.

In some embodiments, a provided compound is useful in treating a blood disorder, e.g., a hemoglobinopathy, such as sickle cell disease or β-thalassemia. For example, while not being bound to any particular mechanism, PRMT5 is a known repressor of γ-globin gene expression, and increased fetal γ-globin (HbF) levels in adulthood are associated with symptomatic amelioration in sickle cell disease and β-thalassemia. See, e.g., Xu et al., Haematologica. (2012) 97:1632-1640; Rank et al. Blood. (2010) 116:1585-1592. Thus in some embodiments, the inhibition of PRMT5 by a provided compound is useful in treating a blood disorder, such as a hemoglobinopathy such as sickle cell disease or β-thalassemia.

In some embodiments, a provided compound is useful to delay the onset of, slow the progression of, or ameliorate the symptoms of, sickle cell disease. In some embodiments, a provided compound is useful to delay the onset of, slow the progression of, or ameliorate the symptoms of, β-thalassemia. In some embodiments, a provided compound could be used in combination with other compounds, drugs, or therapeutics, to treat a hemoglobinopathy such as sickle cell disease or β-thalassemia.

In some embodiments, a provided compound is useful in treating inflammatory and autoimmune disease. PRMT5 is reported to activate NFkB signaling pathway through the methylation of p65. PRMT5 is reported to interact with Death receptor 4 and Death receptor 5 contributing to TRAIL-induced activation of inhibitor or kB kinase (IKK) and nuclear factor-kB (NF-kB). See, e.g., Tanaka et al., Mol. Cancer. Res. (2009) 7:557-569; Wei et al., Proc. Nat'l. Acad. Sci. USA (2013) 110:13516-21.

The term “inflammatory disease” refers to those diseases, disorders or conditions that are characterized by signs of pain (dolor, from the generation of noxious substances and the stimulation of nerves), heat (calor, from vasodilatation), redness (rubor, from vasodilatation and increased blood flow), swelling (tumor, from excessive inflow or restricted outflow of fluid), and/or loss of function (functio laesa, which can be partial or complete, temporary or permanent. Inflammation takes on many forms and includes, but is not limited to, acute, adhesive, atrophic, catarrhal, chronic, cirrhotic, diffuse, disseminated, exudative, fibrinous, fibrosing, focal, granulomatous, hyperplastic, hypertrophic, interstitial, metastatic, necrotic, obliterative, parenchymatous, plastic, productive, proliferous, pseudomembranous, purulent, sclerosing,

seroplastic, serous, simple, specific, subacute, suppurative, toxic, traumatic, and/or ulcerative inflammation.

Exemplary inflammatory diseases include, but are not limited to, inflammation associated with acne, anemia (e.g., aplastic anemia, haemolytic autoimmune anaemia), asthma, arteritis (e.g., polyarteritis, temporal arteritis, periarteritis nodosa, Takayasu's arteritis), arthritis (e.g., crystalline arthritis, osteoarthritis, psoriatic arthritis, gouty arthritis, reactive arthritis, rheumatoid arthritis and Reiter's arthritis), ankylosing spondylitis, amylosis, amyotrophic lateral sclerosis, autoimmune diseases, allergies or allergic reactions, atherosclerosis, bronchitis, bursitis, chronic prostatitis, conjunctivitis, Chagas disease, chronic obstructive pulmonary disease, cermatomyositis, diverticulitis, diabetes (e.g., type I diabetes mellitus, type 2 diabetes mellitus), a skin condition (e.g., psoriasis, eczema, burns, dermatitis, pruritus (itch)), endometriosis, Guillain-Barre syndrome, infection, ischaemic heart disease, Kawasaki disease, glomerulonephritis, gingivitis, hypersensitivity, headaches (e.g., migraine headaches, tension headaches), ileus (e.g., postoperative ileus and ileus during sepsis), idiopathic thrombocytopenic purpura, interstitial cystitis (painful bladder syndrome), gastrointestinal disorder (e.g., selected from peptic ulcers, regional enteritis, diverticulitis, gastrointestinal bleeding, eosinophilic gastrointestinal disorders (e.g., eosinophilic esophagitis, eosinophilic gastritis, eosinophilic gastroenteritis, eosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischaemic colitis, diversion colitis, Behcet's syndrome, indeterminate colitis) and inflammatory bowel syndrome (IBS)), lupus, multiple sclerosis, morphea, myeasthenia gravis, myocardial ischemia, nephrotic syndrome, pemphigus vulgaris, pernicious aneaemia, peptic ulcers, polymyositis, primary biliary cirrhosis, neuroinflammation associated with brain disorders (e.g., Parkinson's disease, Huntington's disease, and Alzheimer's disease), prostatitis, chronic inflammation associated with cranial radiation injury, pelvic inflammatory disease, reperfusion injury, regional enteritis, rheumatic fever, systemic lupus erythematosus, schleroderma, scierodoma, sarcoidosis, spondyloarthopathies, Sjogren's syndrome, thyroiditis, transplantation rejection, tendonitis, trauma or injury (e.g., frostbite, chemical irritants, toxins, scarring, burns, physical injury), vasculitis, vitiligo and Wegener's granulomatosis.

In certain embodiments, the inflammatory disease is an acute inflammatory disease (e.g., for example, inflammation resulting from infection). In certain embodiments, the inflammatory disease is a chronic inflammatory disease (e.g., conditions resulting from asthma, arthritis and inflammatory bowel disease). The compounds may also be useful in treating inflammation associated with trauma and non-inflammatory myalgia. The compounds may also be useful in treating inflammation associated with cancer.

Exemplary autoimmune diseases, include, but are not limited to, arthritis (including rheumatoid arthritis, spondyloarthopathies, gouty arthritis, degenerative joint diseases such as osteoarthritis, systemic lupus erythematosus, Sjogren's syndrome, ankylosing spondylitis, undifferentiated spondylitis, Behcet's disease, haemolytic autoimmune anaemias, multiple sclerosis, amyotrophic lateral sclerosis, amylosis, acute painful shoulder, psoriatic, and juvenile arthritis), asthma, atherosclerosis, osteoporosis, bronchitis, tendonitis, bursitis, skin condition (e.g., psoriasis, eczema, burns, dermatitis, pruritus (itch)), enuresis, eosinophilic disease, gastrointestinal disorder (e.g., selected from peptic ulcers, regional enteritis, diverticulitis, gastrointestinal bleeding, eosinophilic gastrointestinal disorders (e.g., eosinophilic esophagitis, eosinophilic gastritis, eosinophilic gastroenteritis, eosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischaemic colitis, diversion colitis, Behcet's syndrome, indeterminate colitis) and inflammatory bowel syndrome (IBS)), and disorders ameliorated by a gastroprokinetic agent (e.g., ileus, postoperative ileus and ileus during sepsis; gastroesophageal reflux disease (GORD, or its synonym GERD); eosinophilic esophagitis, gastroparesis such as diabetic gastroparesis; food intolerances and food allergies and other functional bowel disorders, such as non-ulcerative dyspepsia (NUD) and non-cardiac chest pain (NCCP, including costo-chondritis)).

In some embodiments, a provided compound is useful in somatic cell reprogramming, such as reprogramming somatic cells into stem cells. See, e.g., Nagamatsu et al., J Biol Chem. (2011) 286:10641-10648. In some embodiments, a provided compound is useful in germ cell development, and are thus envisioned useful in the areas of reproductive technology and regenerative medicine. See, e.g., Ancelin et al., Nat. Cell. Biol. (2006) 8:623-630.

In some embodiments, compounds described herein can prepared using methods as shown in general Schemes 1, 2, and 3 by ring opening of a chiral or racemic epoxide group to form an amino alcohol moiety. A ring opening step can be performed in either direction as shown in Schemes 1 and 2.

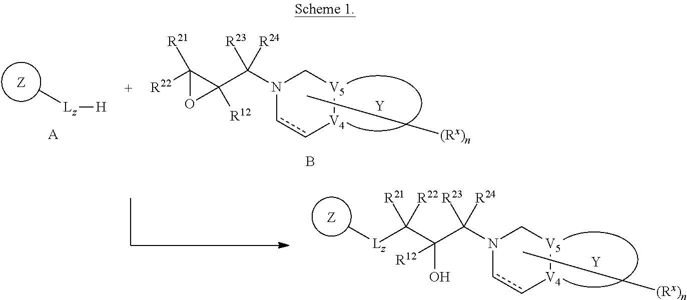

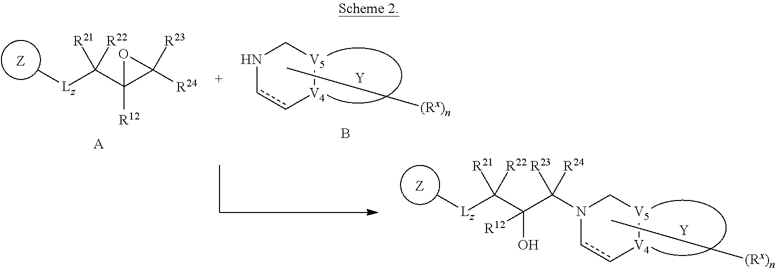

As shown in Scheme 3, epoxide (A) may be hydrolyzed to form intermediate (B). The primary alcohol of intermediate (B) may be changed into a suitable leaving group (e.g., a halogen) for Sn2 displacement to provide intermediate (C). Intermediate (C) may then be reacted with amine (D) to form the final target.

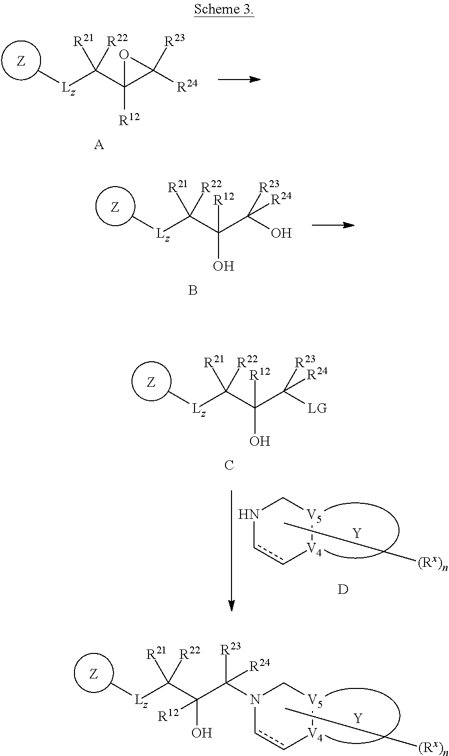

In some embodiments, compounds described herein can prepared using methods shown in general Scheme 4. Compound B can be prepared via ring opening of a chiral or racemic epoxide group. This amino alcohol intermediate can be coupled to form an amide via normal amide coupling methodology using a carboxylic acid A wherein Z₁ is hydrogen or via amination of an ester of intermediate A when Z₁ is an optionally substituted aliphatic group.

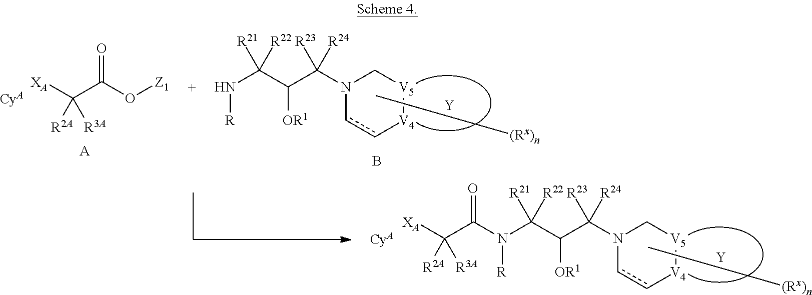

In some embodiments, compounds described herein can prepared using methods shown in general Scheme 5. Compound B can be prepared via ring opening of a chiral or racemic epoxide group. This amino alcohol intermediate can be coupled to form an amide via normal amide coupling methodology using a carboxylic acid A wherein Z₁ is hydrogen or via amination of an ester of intermediate A when Z₁ is an optionally substituted aliphatic group.

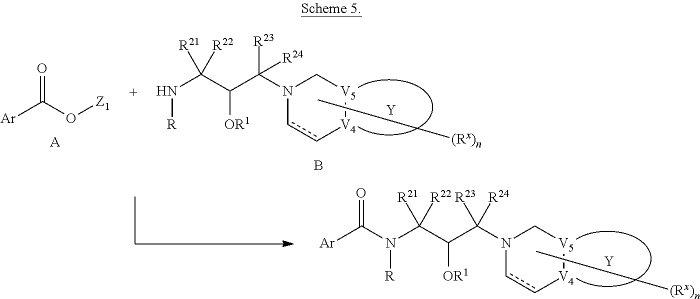

In some embodiments, compounds described herein can prepared using methods shown in general Scheme 6, which describes ring opening of a chiral or racemic epoxide group to form the amino alcohol moiety linker.

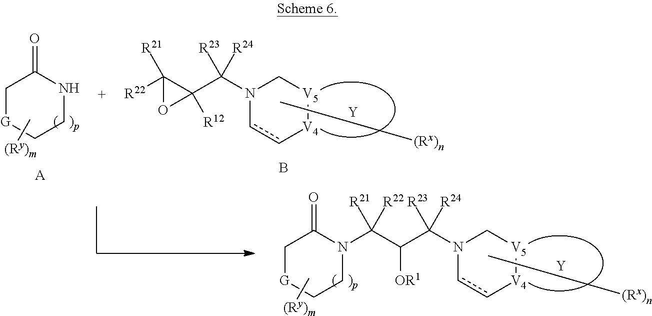

### EXAMPLES

In order that the invention described herein may be more fully understood, the following examples are set forth. It should be understood that these examples are for illustrative purposes onlyd nd are not to be construed as limiting this invention in any manner.

### Synthetic Methods

### Example 1. General Procedure for the Preparation of Compound 1-4, 1-5, and 2-6

### Step 1: Ethyl 2-(quinolin-8-yloxy)acetate

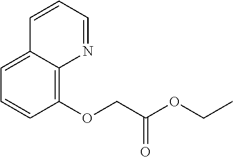 [SMILES: CCOC(=O)COc1cccc2cccnc12]

Ethyl bromoacetate (9.17 mL, 82.67 mmol) and K₂CO₃ (19.04 g, 137.78 mmol) were added to a solution of quinolin-8-ol (10 g, 68.89 mmol) in acetonitrile (100 mL). The mixture was then heated at 80° C. overnight. The solid inorganic residue was filtered off and washed 3 times with acetonitrile (3×25 mL). The combined organics were evaporated to dryness and the deep red residue was purified by flash chromatography on SiO₂. Product rich fractions were combined and evaporated to dryness to afford 15.17 g (95.2%) of ethyl 2-(quinolin-8-yloxy)acetate as a dark red solid. 1H NMR (500 MHz, CDCl₃, δ): 8.95 (dd, J=4.2, 1.7 Hz, 1H), 8.14 (dd, J=8.3, 1.7 Hz, 1H), 7.48-7.40 (m, 3H), 6.98 (dd, J=6.9, 1.9 Hz, 1H), 4.97 (s, 2H), 4.27 (q, J=7.1 Hz, 2H), 1.26 (t, J=7.2 Hz, 3H). LCMS (m/z): 232 (M+1).

### Step 2: N-(2,3-dihydroxypropyl)-2-(quinolin-8-yloxy)acetamide

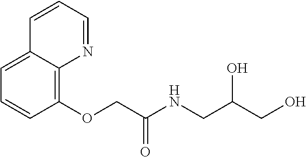 [SMILES: O=C(COc1cccc2cccnc12)NCC(O)CO]

Ethyl 2-(quinolin-8-yloxy)acetate (5 g, 21.62 mmol), 3-aminopropane-1,2-diol (2.56 g, 28.11 mmol) and N,N-diisopropylethylamine (4.9 mL, 28.11 mmol) were heated in ethanol (250 mL) in a pressure vessel at 100° C. overnight. LCMS of the reaction mixture indicated 75% conversion to the product. Further 3-aminopropane-1,2-diol (0.5 eq.) and N,N-diisopropylethylamine (0.5 eq.) were added and the reaction was continued as before. The total reaction time was 48 h. LCMS analysis showed 95% conversion to the desired product. The solvent was removed in vacuo and the residue was purified by flash chromatography on SiO₂ to give 4.42 g (74%) of N-(2,3-dihydroxypropyl)-2-(quinolin-8-yloxy)acetamide as a yellow solid. 1H NMR (500 MHz, DMSO-d₆, δ): 8.91 (dd, J=4.1, 1.7 Hz, 1H), 8.36 (dd, J=8.3, 1.7 Hz, 1H), 8.28 (t, J=5.6 Hz, 1H), 7.62-7.56 (m, 2H), 7.53 (t, J=7.9 Hz, 1H), 7.27 (dd, J=7.7, 1.1 Hz, 1H), 4.84 (d, J=5.0 Hz, 1H), 4.75 (s, 2H), 4.57 (t, J=5.8 Hz, 1H), 3.54 (dq, J=10.6, 5.3 Hz, 1H), 3.37 (m, 1H), 3.33-3.25 (m, 2H), 3.12-3.05 (m, 1H). LCMS (m/z): 277 (M+1).

### Step 3: N-(2-hydroxy-3-iodopropyl)-2-(quinolin-8-yloxy)acetamide

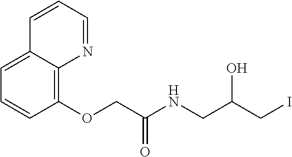 [SMILES: O=C(COc1cccc2cccnc12)NCC(O)CI]

To a stirred mixture of N-(2,3-dihydroxypropyl)-2-(quinolin-8-yloxy)acetamide (4.42 g, 16 mmol), triphenylphosphane (5.04 g, 19.2 mmol) and 1H-imidazole (1.31 g, 19.2 mmol) in dimethylformamide (75 mL) was added a solution of iodine (4.87 g, 19.2 mmol) in dimethylformamide (10 mL) dropwise at 0° C. The mixture was allowed to warm to ambient temperature and was left to stir overnight. The solvent was removed in vacuo and the residue triturated with dichloromethane. The precipitated solid was filtered off, washed with dichloromethane, and dried under vacuum to afford 2.91 g (47.1%) of N-(2-hydroxy-3-iodopropyl)-2-(quinolin-8-yloxy)acetamide as a yellow solid. 1H NMR (500 MHz, DMSO-d₆, δ): 8.91 (dd, J=4.1, 1.7 Hz, 1H), 8.37 (dd, J=8.3, 1.7 Hz, 2H), 7.64-7.56 (m, 2H), 7.53 (t, J=7.9 Hz, 1H), 7.26 (dd, J=7.7, 1.0 Hz, 1H), 5.46 (d, J=5.1 Hz, 1H), 4.76 (s, 2H), 3.54 (h, J=5.5 Hz, 1H), 3.31-3.29 (m, 1H), 3.26 (dd, J=10.2, 4.7 Hz, 1H), 3.22-3.13 (m, 2H). LCMS (m/z): 387 (M+1).

### Step 4: Preparation of Compounds 1-4, 1-5, and 2-6 via N-alkylation with N-(2-hydroxy-3-iodopropyl)-2-(quinolin-8-yloxy)acetamide

All of the amines were converted to the corresponding free bases by SCX-2 column prior to the reaction. N-(2-hydroxy-3-iodopropyl)-2-(quinolin-8-yloxy)acetamide (1 eq.) and the selected amine (free base, 2 eq.) were suspended in methanol and the mixture was heated at 80° C. for approximately 24 hrs. Upon consumption of starting material by LCMS, the solvent was removed in vacuo and the residue submitted directly for basic pH prep HPLC.

### N-(2-hydroxy-3-{4H,5H,6H,7H-thieno[3,2-c]pyridin-5-yl}propyl)-2-(quinolin-8-yloxy)acetamide (Compound 1-4)

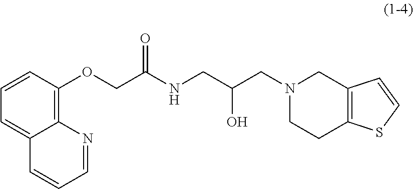 [SMILES: O=C(COc1cccc2cccnc12)NCC(O)CN1CCc2sccc2C1]

N-(2-hydroxy-3-iodopropyl)-2-(quinolin-8-yloxy)acetamide (1 eq), 4,5,6,7-tetrahydrothieno[3,2-c]pyridine (free base, 2 eq) were suspended in MeOH and the mixture was heated at 80° C. for about 24 hrs in total. Once no starting material left by LC/MS, the solvent was removed and the residue submitted directly for High pH prep HPLC. Scale: 140 mg of iodide. Yield=45 mg (32%). ¹H NMR (500 MHz, DMSO-d6) δ 8.90 (dd, J=4.1, 1.7 Hz, 1H), 8.36 (dd, J=8.3, 1.7 Hz, 1H), 8.29 (t, J=5.7 Hz, 1H), 7.62-7.56 (m, 2H), 7.52 (t, J=7.9 Hz, 1H), 7.29-7.22 (m, 2H), 6.75 (d, J=5.1 Hz, 1H), 4.87 (s, 1H), 4.76 (s, 2H), 3.79 (p, J=6.0 Hz, 1H), 3.47 (s, 2H), 3.42-3.37 (m, 1H), 3.21-3.10 (m, 1H), 2.78-2.66 (m, 4H), 2.44 (d, J=6.1 Hz, 2H). MS m/z 398 (M++H).

### N-(2-hydroxy-3-{4H,5H,6H,7H-thieno[2,3-c]pyridin-6-yl}propyl)-2-(quinolin-8-yloxy)acetamide (Compound 1-5)

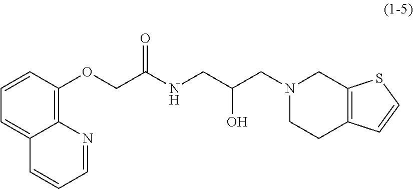 [SMILES: O=C(COc1cccc2cccnc12)NCC(O)CN1CCc2ccsc2C1]

N-(2-hydroxy-3-iodopropyl)-2-(quinolin-8-yloxy)acetamide (1 eq), 4,5,6,7-tetrahydrothieno[2,3-c]pyridine (free base, 2 eq) were suspended in MeOH and the mixture was heated at 80° C. for about 24 hrs in total. Once no starting material left by LC/MS, the solvent was removed and the residue submitted directly for High pH prep HPLC. Scale: 140 mg of iodide. Yield=42 mg (29%). ¹H NMR (500 MHz, DMSO-d6) δ 8.89 (dd, J=4.1, 1.7 Hz, 1H), 8.36 (dd, J=8.3, 1.6 Hz, 1H), 8.28 (t, J=5.7 Hz, 1H), 7.62-7.55 (m, 2H), 7.52 (t, J=7.9 Hz, 1H), 7.29-7.21 (m, 2H), 6.77 (d, J=5.0 Hz, 1H), 4.86 (s, 1H), 4.75 (s, 2H), 3.77 (p, J=5.9 Hz, 1H), 3.60 (s, 2H), 3.40-3.35 (m, 1H), 3.19-3.11 (m, 1H), 2.73-2.62 (m, 2H), 2.62-2.56 (m, 2H), 2.44 (d, J=6.1 Hz, 2H). MS m/z 398 (M++H).

### N-[2-hydroxy-3-(5,6,7,8-tetrahydro-1,6-naphthyridin-6-yl)propyl]-2-(quinolin-8-yloxy)acetamide (Compound 2-6)

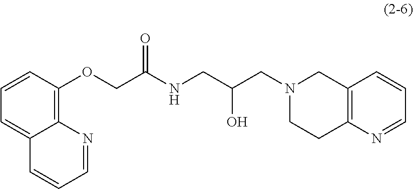 [SMILES: O=C(COc1cccc2cccnc12)NCC(O)CN1CCc2ncccc2C1]

N-(2-hydroxy-3-iodopropyl)-2-(quinolin-8-yloxy)acetamide and 5,6,7,8-tetrahydro-1,6-naphthyridine (97.28 mg, 0.73 mmol) were suspended in methanol and the mixture was heated at 80° C. for 24 hrs. The solvent was then removed in vacuo and the residue purified by flash chromatography on SiO₂, eluting with a gradient of 0 to 10% 7M NH₃/methanol in dichloromethane. The product rich fractions were combined and concentrated in vacuo. The residue was further purified by basic pH prep HPLC to afford 27 mg (19%) of N-[2-hydroxy-3-(5,6,7,8-tetrahydro-1,6-naphthyridin-6-yl)propyl]-2-(quinolin-8-yloxy)acetamide as a white powder after evaporation and drying under high vacuum.

Scale: 140 mg of iodide; Yield=27 mg (19%); 1H NMR (500 MHz, CD₃OD, δ): 8.86 (dd, J=4.3, 1.7 Hz, 1H), 8.37 (dd, J=8.3, 1.6 Hz, 1H), 8.22 (dd, J=4.9, 1.4 Hz, 1H), 7.63-7.51 (m, 3H), 7.45 (d, J=6.7 Hz, 1H), 7.25 (dd, J=7.5, 1.4 Hz, 1H), 7.12 (dd, J=7.7, 4.9 Hz, 1H), 4.73 (d, J=1.2 Hz, 2H), 4.15-4.06 (m, 1H), 3.72 (s, 2H), 3.54 (dd, J=13.6, 5.1 Hz, 1H), 3.42 (dd, J=13.6, 6.8 Hz, 1H), 2.97 (t, J=5.7 Hz, 2H), 2.90 (t, J=5.6 Hz, 2H), 2.69-2.58 (m, 2H). LCMS (m/z): 393 (M+1).

### Example 2. General Procedure in the Preparation of Compounds 2-5, 2-7, 2-8, and 3-10

All of the amines (200 mg of the HCl salt) were converted to the corresponding free base by SCX-2 column prior to the reaction. A mixture of the amine and N-(2-hydroxy-3-iodopropyl)-2-(quinolin-8-yloxy)acetamide (140 mg, 0.36 mmol) were suspended in methanol (5 mL) and heated at 80° C. for 24 h. The solvent was partially evaporated in vacuo and the residue purified by flash chromatography on SiO₂, once with 0 to 10% 7M ammonia in methanol/dichloromethane, and twice further with 0-10% methanol/dichloromethane.

### N-[2-hydroxy-3-(5,6,7,8-tetrahydro-1,7-naphthyridin-7-yl)propyl]-2-(quinolin-8-yloxy)acetamide (Compound 2-5)

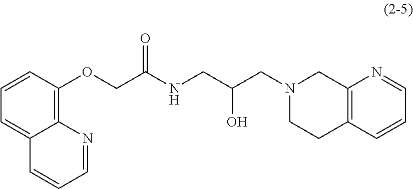 [SMILES: O=C(COc1cccc2cccnc12)NCC(O)CN1CCc2cccnc2C1]

Only 2 columns were run, the second included washing with 10% 7M ammonia in methanol in dichloromethane followed by slurrying with minimal methanol. Yield=17 mg (12%); ¹H NMR (500 MHz, DMSO-d₆, δ): 8.89 (dd, J=4.1, 1.7 Hz, 1H), 8.35 (dd, J=8.3, 1.6 Hz, 1H), 8.30-8.25 (m, 2H), 7.62-7.55 (m, 2H), 7.51 (t, J=7.9 Hz, 1H), 7.46-7.41 (m, 1H), 7.27-7.22 (m, 1H), 7.11 (dd, J=7.7, 4.7 Hz, 1H), 4.87 (d, J=4.8 Hz, 1H), 4.74 (s, 2H), 3.82 (h, J=6.3, 5.7 Hz, 1H), 3.67-3.56 (m, 2H), 3.42-3.35 (m, 1H), 3.22-3.14 (m, 1H), 2.77 (t, J=5.5 Hz, 2H), 2.68 (dq, J=11.3, 5.4 Hz, 2H), 2.46 (dd, J=6.1, 1.7 Hz, 2H). LCMS (m/z): 393 (M+1).

### N-[2-hydroxy-3-(1,2,3,4-tetrahydro-2,6-naphthyridin-2-yl)propyl]-2-(quinolin-8-yloxy)acetamide (Compound 2-7)

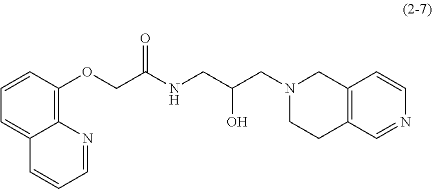 [SMILES: O=C(COc1cccc2cccnc12)NCC(O)CN1CCc2cnccc2C1]

Yield=16 mg (11%); ¹H NMR (500 MHz, CD₃OD, δ): 8.87 (dd, J=4.3, 1.6 Hz, 1H), 8.39 (dd, J=8.3, 1.6 Hz, 1H), 8.17 (s, 1H), 8.14 (d, J=5.2 Hz, 1H), 7.64-7.59 (m, 2H), 7.58-7.54 (m, 1H), 7.26 (dd, J=7.5, 1.2 Hz, 1H), 7.05 (d, J=5.2 Hz, 1H), 4.78-4.70 (m, 2H), 4.13-4.06 (m, 1H), 3.72 (s, 2H), 3.53 (dd, J=13.6, 5.2 Hz, 1H), 3.43 (dd, J=13.6, 6.6 Hz, 1H), 2.91-2.81 (m, 4H), 2.66 (dd, J=12.9, 5.1 Hz, 1H), 2.61 (dd, J=12.9, 7.2 Hz, 1H). LCMS (m/z): 393 (M+1).

### N-[2-hydroxy-3-(1,2,3,4-tetrahydro-2,7-naphthyridin-2-yl)propyl]-2-(quinolin-8-yloxy)acetamide (Compound 2-8)

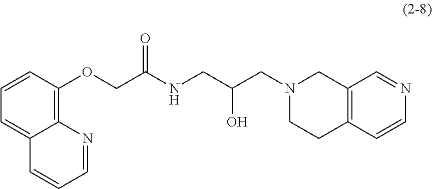 [SMILES: O=C(COc1cccc2cccnc12)NCC(O)CN1CCc2ccncc2C1]

Scale=2×140 mg. Only the first 2 columns were run; Yield=19 mg (6%); ¹H NMR (500 MHz, CD₃OD, δ): 8.87 (dd, J=4.3, 1.6 Hz, 1H), 8.39 (dd, J=8.3, 1.6 Hz, 1H), 8.17 (s, 1H), 8.15 (d, J=5.1 Hz, 1H), 7.64-7.58 (m, 2H), 7.58-7.54 (m, 1H), 7.26 (dd, J=7.5, 1.2 Hz, 1H), 7.07 (d, J=5.1 Hz, 1H), 4.78-4.71 (m, 2H), 4.14-4.07 (m, 1H), 3.72 (s, 2H), 3.54 (dd, J=13.6, 5.2 Hz, 1H), 3.43 (dd, J=13.6, 6.7 Hz, 1H), 2.90 (t, J=5.8 Hz, 2H), 2.83 (td, J=6.9, 6.1, 3.7 Hz, 2H), 2.67 (dd, J=12.9, 5.1 Hz, 1H), 2.62 (dd, J=12.9, 7.2 Hz, 1H). LCMS (m/z): 393 (M+1).

### N-(2-hydroxy-3-{3H,4H,5H,6H,7H-imidazo[4,5-c]pyridin-5-yl}propyl)-2-(quinolin-8-yloxy)acetamide (Compound 3-10)

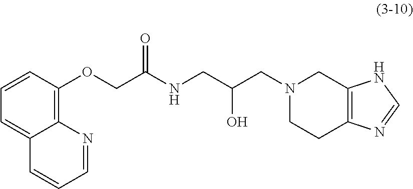 [SMILES: O=C(COc1cccc2cccnc12)NCC(O)CN1CCc2nc[nH]c2C1]

The mix of N-(2-hydroxy-3-iodopropyl)-2-(quinolin-8-yloxy)acetamide (140 mg; 0.36 mmol) and 4,5,6,7-tetrahydro-3H-imidazo[4,5-c]pyridine (2 eq) were suspended in MeOH (5 mL) and heated at 80° C. for 24 h. The solvent was partially evaporated and the residue chromatographed with 0-10% 7M ammonia in MeOH/DCM. Yield=50 mg (35%). ¹H NMR (500 MHz, Methanol-d4) δ 8.88 (dd, J=4.3, 1.7 Hz, 1H), 8.38 (dd, J=8.3, 1.6 Hz, 1H), 7.63-7.52 (m, 3H), 7.47 (s, 1H), 7.26 (dd, J=7.4, 1.4 Hz, 1H), 4.75 (s, 2H), 4.11-4.05 (m, 1H), 3.66-3.58 (m, 2H), 3.50 (dd, J=13.6, 5.0 Hz, 1H), 3.40 (dd, J=13.6, 6.8 Hz, 1H), 2.94-2.84 (m, 2H), 2.74-2.61 (m, 4H). MS m/z 382 (M+H).

### Example 3. Preparation of 1-(3-((cyclopentylamino)methyl)phenoxy)-3-(3-phenyl-6,7-dihydro-1H-pyrazolo[4,3-c]pyridin-5(4H)-yl)propan-2-ol (Compound 3-9)

### Step 1: 3-(oxiran-2-ylmethoxy)benzaldehyde

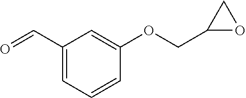 [SMILES: O=Cc1cccc(OCC2CO2)c1]

Sodium hydride (60% in mineral oil, 983 mg, 24.57 mmol) was added in portions to a stirred and cooled (0° C.) solution of 3-hydroxybenzaldehyde (2.0 g, 16.38 mmol) in dimethylformamide (30 mL) and the mixture was then stirred at 0° C. for 0.5 h before a solution of 2-(bromomethyl)oxirane (2.69 mg, 19.65 mmol) in dimethylformamide (5 mL) was added. Stirring was continued for 5 h at ambient temperature. The solvent then was removed in vacuo and the residue was dissolved in ethyl acetate (100 mL), washed with water (30 mL) and the separated organic layer was dried over sodium sulfate. The solution was filtered, and the filtrate concentrated in vacuo. The crude product (2.1 g, 72%) was used in next step without further purification. LCMS (m/z): 179.1 (M+1).

### Step 2: N-(3-(oxiran-2-ylmethoxy)benzyl)cyclopentanamine

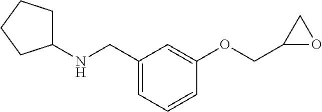 [SMILES: c1cc(CNC2CCCC2)cc(OCC2CO2)c1]

To a solution of 3-(oxiran-2-ylmethoxy)benzaldehyde (1.0 g, 5.61 mmol) in methanol (15 mL) was added cyclopentanamine (502 mg, 5.89 mmol). After addition, the mixture was stirred at ambient temperature for 4 h and then NaBH₄ (318 mg, 8.42 mmol) was added. The resulting mixture was stirred for another 1 h before quenching by addition of aqueous 1N HCl, adjusting to pH 6-7. The solution was diluted with ethyl acetate (30 mL) and the organic layer was washed with water (10 mL). The separated organic layer was dried over sodium sulfate, filtered, and concentrated in vacuo. The crude product (1.1 g, 79%) was used in next step without further purification. LCMS (m/z): 248.2 (M+1).

### Step 3: tert-butyl cyclopentyl(3-(oxiran-2-ylmethoxy)benzyl)carbamate

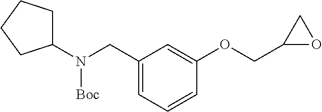 [SMILES: CC(C)(C)OC(=O)N(Cc1cccc(OCC2CO2)c1)C1CCCC1]

To a solution of N-(3-(oxiran-2-ylmethoxy)benzyl)cyclopentanamine (1.0 g, 4.04 mmol) in tetrahydrofuran (30 mL) was added Boc₂O (1.32 g, 6.06 mmol) and triethylamine (614 mg, 6.06 mmol). The reaction mixture was stirred at ambient temperature for 12 h. The solvent was then removed in vacuo and the residue was dissolved in ethyl acetate and washed with water. The separated organic layer was dried over sodium sulfate, filtered, and concentrated in vacuo. The resulting crude material was purified by flash chromatography on SiO₂ with 10-30% of ethyl acetate in hexanes to afford the final product (1.2 g, 86%). LCMS (m/z): 348.2 (M+1).

### Step 4: tert-butyl cyclopentyl(3-(2-hydroxy-3-(3-phenyl-6,7-dihydro-1H-pyrazolo[4,3-c]pyridin-5(4H)-yl)propoxy)benzyl)carbamate

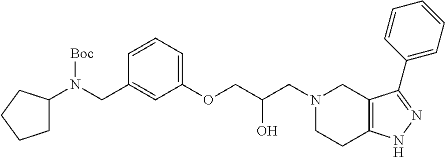 [SMILES: CC(C)(C)OC(=O)N(Cc1cccc(OCC(O)CN2CCc3[nH]nc(-c4ccccc4)c3C2)c1)C1CCCC1]

A solution of tert-butyl cyclopentyl(3-(oxiran-2-ylmethoxy)benzyl)carbamate (200 mg, 0.576 mmol) and 3-phenyl-4,5,6,7-tetrahydro-1H-pyrazolo[4,3-c]pyridine (114.8 mg, 0.576 mmol) in ethanol (3 mL) was heated to 100° C. under microwave for 30 min. The solution was concentrated and the residue was used in the next step without further purification.

### Step 5: 1-(3-((cyclopentylamino)methyl)phenoxy)-3-(3-phenyl-6,7-dihydro-1H-pyrazolo[4,3-c]pyridin-5(4H-yl)propan-2-ol (Compound 3-9

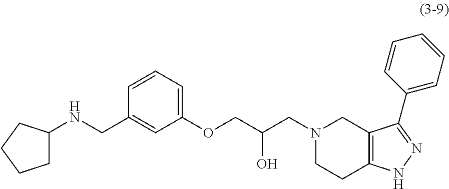 [SMILES: OC(COc1cccc(CNC2CCCC2)c1)CN1CCc2[nH]nc(-c3ccccc3)c2C1]

To a solution of tert-butyl cyclopentyl(3-(2-hydroxy-3-(3-phenyl-6,7-dihydro-1H-pyrazolo[4,3-c]pyridin-5(4H)-yl)propoxy)benzyl)carbamate (220 mg, 0.462 mmol) in dichloromethane (9 mL) was added TFA (3 mL). The resulting solution was stirred for 2 h and evaporated. The crude product was purified by preparative HPLC to give the product as a TFA salt (17.9 mg, 8.7%). ¹H NMR (400 MHz, CD₃OD, δ): 7.63 (d, J=7.2 Hz, 2H), 7.57-7.52 (m, 3H), 7.39 (t, J=8.0 Hz, 1H), 7.22 (s, 1H), 7.10 (d, J=7.6 Hz, 1H), 7.05 (d, J=8.4 Hz, 1H), 4.80-4.70 (m, 2H), 4.56 (s, 1H), 4.17 (s, 2H), 4.11 (d, J=4.8 Hz, 2H), 3.73-3.65 (m, 2H), 3.59-3.54 (m, 2H), 2.19-2.10 (m, 2H), 1.84-1.81 (m, 2H), 1.75-1.62 (m, 4H). LCMS (m/z): 446.9 (M+1).

### Example 4: Preparation of 1-(3-((cyclopentylamino)methyl)phenoxy)-3-(7,8-dihydro-1,6-naphthyridin-6(5H)-yl)propan-2-ol (Compound 2-1)

### Step 1: tert-butyl cyclopentyl(3-(3-(7,8-dihydro-1,6-naphthyridin-6(5H)-yl)-2-hydroxypropoxy)benzyl)carbamate

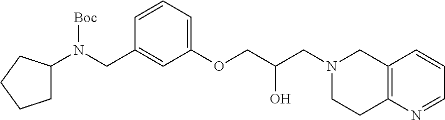 [SMILES: CC(C)(C)OC(=O)N(Cc1cccc(OCC(O)CN2CCc3ncccc3C2)c1)C1CCCC1]

The solution of tert-butyl cyclopentyl(3-(oxiran-2-ylmethoxy)benzyl)carbamate (200 mg, 0.576 mmol) and 5,6,7,8-tetrahydro-1,6-naphthyridine (77.3 mg, 0.576 mmol) in ethanol (3 mL) was heated to 100° C. under microwave for 30 min. The solution was concentrated in vacuo and the residue was used in next step without further purification.

### Step 2: 1-(3-((cyclopentylamino)methyl)phenoxy)-3-(7,8-dihydro-1,6-naphthyridin-6(5H)-yl)propan-2-ol(Compound 2-1)

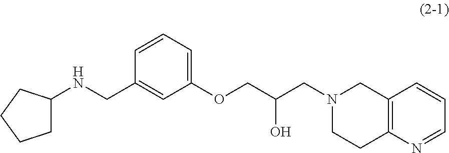 [SMILES: OC(COc1cccc(CNC2CCCC2)c1)CN1CCc2ncccc2C1]

To a solution of tert-butyl cyclopentyl(3-(3-(7,8-dihydro-1,6-naphthyridin-6(5H)-yl)-2-hydroxypropoxy)benzyl)carbamate (250 mg, 0.519 mmol) in dichloromethane (9 mL) was added TFA (3 mL). The solution was stirred for 2 h and evaporated. The crude product was purified by preparative HPLC to give the HCl salt of the desired target compound (21.7 mg, 10.9%). ¹H NMR (400 MHz, CD₃OD, δ): 8.86 (d, J=5.6 Hz, 1H), 8.57 (d, J=7.6 Hz, 1H), 8.05-8.01 (m, 1H), 7.40 (t, J=7.6 Hz, 1H), 7.27 (s, 1H), 7.12-7.05 (m, 2H), 5.04 (s, 1H), 4.94 (s, 1H), 4.64-4.61 (m, 1H), 4.18 (s, 2H), 4.13 (d, J=5.2 Hz, 2H), 4.09-4.01 (m, 1H), 3.76-3.55 (m, 6H), 2.20-2.13 (m, 2H), 1.84-1.64 (m, 6H). LCMS (m/z): 382.0 (M+1).

### Example 5: Preparation of 1-(3-(1-methyl-1H-benzo[d]imidazol-6-yl)phenoxy)-3-(3-phenyl-6,7-dihydro-1H-pyrazolo[4,3-c]pyridin-5(4H)-yl)propan-2-ol (Compound 3-6)

### Step 1: 5-bromo-N-methyl-2-nitroaniline

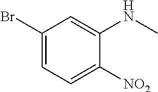

To a solution of 4-bromo-2-fluoro-1-nitrobenzene (10 g, 45.7 mmol) in DMSO (50 mL) were added triethylamine (18.47 g, 183 mmol) and methylamine hydrochloride (6.1 g, 91.4 mmol). The reaction mixture was heated at 120° C. for 3 hours. After cooling, the mixture was extracted with ethyl acetate (100 mL×3), the combined organic layers washed with brine, dried over Na₂SO₄, and concentrated in vacuo to yield a crude product which was used in next step without further purification (10.5 g, 98%). LCMS (m/z): 231.1 (M+1).

### Step 2: 5-bromo-N1-methylbenzene-1,2-diamine

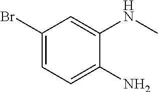

To a solution of 5-bromo-N-methyl-2-nitroaniline (10 g, 43.5 mmol) in ethanol/water (700 mL) were added Fe (14.6 g, 261 mmol) and ammonium chloride (14 g, 261 mmol). The reaction mixture was heated at 60° C. under the atmosphere of nitrogen for 4 h. The solid was removed by filtration. The filtrate was concentrated in vacuo then dissolved in ethyl acetate (300 mL), washed with brine, dried over Na₂SO₄, and concentrated in vacuo to give the crude product which was used in next step without further purification (7.9 g, 91%). LCMS (m/z): 202.1 (M+1).

### Step 3: 6-bromo-1-methyl-1H-benzo[d]imidazole

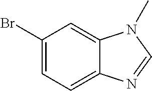

To a solution of 5-bromo-N1-methylbenzene-1,2-diamine (7.4 g, 37 mmol) in HC(OMe)₃ (100 mL) was added TsOH (0.36 g, 1.9 mmol). The reaction mixture was heated at 100° C. for 4 h and the solvent was removed in vacuo. The residue was dissolved in ethyl acetate (200 mL), washed with brine, dried over Na₂SO₄, and concentrated. The crude product was used in next step without further purification (7.3 g, 93%). LCMS (m/z): 212.1 (M+1).

### Step 4: 1-methyl-6-(4,4,5,5-tetramethyl-1,3,2-dioxaborolan-2-yl)-1H-benzo[d]imidazole

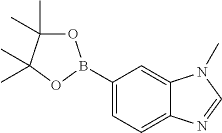 [SMILES: Cn1cnc2ccc(B3OC(C)(C)C(C)(C)O3)cc21]

To a solution of 6-bromo-1-methyl-1H-benzo[d]imidazole (5 g, 23.8 mmol) in dioxane (60 mL) was added 4,4,4′,4′,5,5,5′,5′-octamethyl-2,2′-bi(1,3,2-dioxaborolane) (9.1 g, 35.7 mmol), Pd(dppf)Cl₂ (0.5 g) and KOAc (4.67 g, 47.6 mmol). The reaction mixture was heated at 100° C. under nitrogen for 3 h until the reaction appeared complete by TLC analysis. The solvent was evaporated and the resulting residue was purified by flash chromatography on SiO₂ to afford the desired product (6 g, 93%) as a pale solid. LCMS (m/z): 259.1 (M+1).

### Step 5: 3-(1-methyl-1H-benzo[d]imidazol-6-yl)phenol

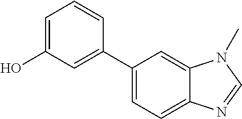 [SMILES: Cn1cnc2ccc(-c3cccc(O)c3)cc21]

To a solution of 1-methyl-6-(4,4,5,5-tetramethyl-1,3,2-dioxaborolan-2-yl)-1H-benzo[d]imidazole (6 g, 23.1 mmol) in dioxane/water (50 mL) were added 3-bromophenol (4.8 g, 27.7 mmol), Pd(dppf)Cl₂ (0.3 g), and Cs₂CO₃ (15 g, 46.2 mmol). The reaction mixture was heated at 100° C. under nitrogen for 3 h. The solvent was evaporated and residue was purified by flash chromatography on SiO₂ to afford 3-(1-methyl-1H-benzo[d]imidazol-6-yl)phenol (4.8 g, 92%). LCMS (m/z): 225.1 (M+1).

### Step 6: 1-methyl-6-(3-(oxiran-2-ylmethoxy)phenyl)-1H-benzo[d]imidazole

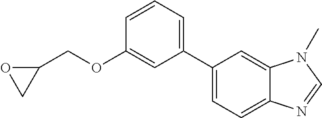 [SMILES: Cn1cnc2ccc(-c3cccc(OCC4CO4)c3)cc21]

To a solution of sodium hydride (60% in mineral oil, 268 mg, 6.69 mmol) in dimethylformamide (5 mL) was added 3-(1-methyl-1H-benzo[d]imidazol-6-yl)phenol (500 mg, 2.23 mmol) at ambient temperature. After addition, the resulting solution was stirred for 30 min and then 2-(chloromethyl)oxirane (246 mg, 2.68 mmol) was added. The mixture was stirred for 16 h until the reaction was complete. The mixture was the treated with water (50 mL), the aqueous layer extracted with ethyl acetate (30 mL×2), and the combined organic layers washed with brine (30 mL), dried over Na₂SO₄, and concentrated in vacuo to give the title compound (500 mg, 80%) as colorless oil which was used in next step without further purification.

### Step 7: 1-(3-(1-methyl-1H-benzo[d]imidazol-6-yl)phenoxy)-3-(3-phenyl-6,7-dihydro-1H-pyrazolo[4,3-c]pyridin-5(4H)-yl)propan-2-ol (Compound 3-6)

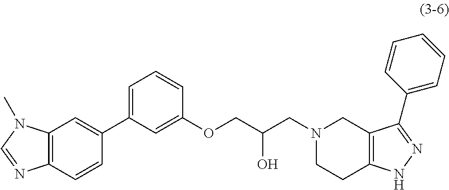 [SMILES: Cn1cnc2ccc(-c3cccc(OCC(O)CN4CCc5[nH]nc(-c6ccccc6)c5C4)c3)cc21]

To a solution of 1-methyl-6-(3-(oxiran-2-ylmethoxy)phenyl)-1H-benzo[d]imidazole (100 mg, 0.357 mmol) in methanol (5 mL) was added 3-phenyl-4,5,6,7-tetrahydro-1H-pyrazolo[4,3-c]pyridine (84 mg, 0.357 mmol) at 25° C. The mixture was heated at reflux for 16 h until the reaction was complete. After cooling to ambient temperature, the mixture was evaporated to dryness under reduced pressure and the crude residue was purified by preparative HPLC to yield the TFA salt of the desired product 1-(3-(1-methyl-1H-benzo[d]imidazol-6-yl)phenoxy)-3-(3-phenyl-6,7-dihydro-1H-pyrazolo[4,3-c]pyridin-5(4H)-yl)propan-2-ol as a white solid (80 mg, 46.7%). ¹H NMR (400 MHz, CD₃OD, δ): 9.39 (s, 1H), 8.15 (s, 1H), 7.96 (s, 1H), 7.60-7.35 (m, 8H), 7.08 (dd, J₁=8.0 Hz, J₂=1.6 Hz, 1H), 4.72 (br. s, 1H), 4.59-4.55 (m, 1H), 4.19 (s, 3H), 4.17-4.16 (m, 2H), 3.87 (br. s, 2H), 3.67-3.56 (m, 2H), 3.25-3.22 (m, 2H). LCMS (m/z): 480.3 (M+1).

### Example 6: Preparation of 1-(7,8-dihydro-1,6-naphthyridin-6(5H)-yl)-3-(3-(1-methyl-1H-benzo[d]imidazol-6-yl)phenoxy)propan-2-ol (Compound 2-4)

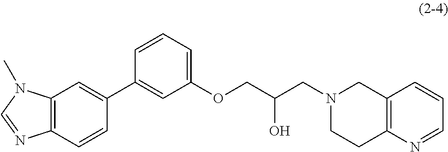 [SMILES: Cn1cnc2ccc(-c3cccc(OCC(O)CN4CCc5ncccc5C4)c3)cc21]

To the solution of 1-methyl-6-(3-(oxiran-2-ylmethoxy)phenyl)-1H-benzo[d]imidazole (100 mg, 0.38 mmol) in EtOH (5 mL) was added 5,6,7,8-tetrahydro-1,6-naphthyridine (80 mg, 0.6 mmol) and the resulting solution was then heated at 80° C. for 5 h. The solvent was then evaporated to dryness and the residue purified by Pre-HPLC to afford the desired product (40 mg, yield: 25.4%). ¹H NMR (400 MHz, CD₃OD, δ): 8.41 (d, J=4.4 Hz, 1H), 8.29 (br.s, 1 h), 7.78 (S, 1H), 7.74 (d, J=8.4 Hz, 1H), 7.63 (d, J=8.0 Hz, 1H), 7.59 (d, J=8.4 Hz, 1H), 7.42-7.27 (m, 4H), 6.99 (d, J=8.0 Hz, 1H), 4.42 (br.s, 1H), 4.20-4.13 (m, 4H), 3.93 (s, 3H), 3.39 (br.s, 2H), 3.18 (br.s, 4H); LCMS:415.2 (M+1).

### Example 7: Preparation of (S)-6-((1-acetylpiperidin-4-yl)amino)-N-(3-(5,6-dihydro-1,7-naphthyridin-7(8H)-yl)-2-hydroxypropyl)pyrimidine-4-carboxamide (Compound 2-11)

### Step 1: methyl 3-methylpicolinate

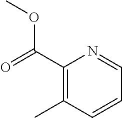

A mixture of 2-bromo-3-methylpyridine (5.0 g, 29.0 mmol), Pd(dppf)Cl₂ (2.1 g, 2.9 mmol), and triethylamine (8.8 g, 87 mmol) in methanol (250 mL) was stirred at 80° C. under CO atmosphere (50 psi) for 16 h. The mixture was filtered and the filtrate concentrated in vacuo, then purified by column chromatography on SiO₂ to give the desired product (4.1 g, 93.6%). LCMS (m/z): 152.0 (M+1).

### Step 2: methyl 3-(bromomethyl)picolinate

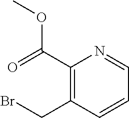

A mixture of methyl 3-methylpicolinate (4.1 g, 27.1 mmol), NBS (5.8 g, 32.5 mmol), and AIBN (100 mg, 0.61 mmol) in carbon tetrachloride (55 mL) was stirred at 90° C. for 16 h under nitrogen. The mixture was filtered and concentrated before being purified by column chromatography to give the desired product (5.0 g, 80.6%). 1H NMR (400 MHz, CDCl₃, δ): 8.67 (dd, J=1.6, 4.6 Hz, 1H), 7.91 (dd, J=1.5, 7.9 Hz, 1H), 7.48 (dd, J=4.6, 7.9 Hz, 1H), 4.95 (s, 2H), 4.07-4.03 (m, 3H). LCMS (m/z): 229.9 (M+1).

### Step 3: methyl 3-(cyanomethyl)picolinate

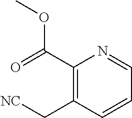

To a solution of methyl 3-(bromomethyl)picolinate (6.0 g, 26.0 mmol) in acetonitrile (200 mL) was added TBAF (10.2 g, 39.0 mmol) and TMSCN (5.2 g, 52.0 mmol) at 0° C. After addition, the mixture was stirred at ambient temperature for 16 h, until completion of the reaction by LCMS analysis. The mixture was diluted with dichloromethane (300 mL) and washed with brine (60 mL×2). The organic layer was then dried over Na₂SO₄ and concentrated. The crude product was purified by flash chromatography on SiO₂ to give the desired product (2.3 g, 50.3%). 1H NMR (400 MHz, CDCl₃, δ): 8.76 (dd, J=1.5, 4.6 Hz, 1H), 8.04 (td, J=0.8, 8.0 Hz, 1H), 7.58 (dd, J=4.6, 8.0 Hz, 1H), 4.31 (s, 2H), 4.04 (s, 3H). LCMS (m/z): 177.0 (M+1).

### Step 4: 6,7-dihydro-1,7-naphthyridin-8(5H)-one

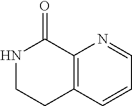 [SMILES: O=C1NCCc2cccnc21]

A mixture of methyl 2-(cyanomethyl)nicotinate (2.3 g, 13.0 mmol) and Raney Ni (400 mg) in a mixture solution of ethanol (40 mL) and water (40 mL) was hydrogenated at 50° C. under hydrogen at 50 psi for 16 h. The mixture was then filtered, and the filtrate concentrated to give the desired product which was used without further purification (2.1 g, 109.4%).

### Step 5: 5,6,7,8-tetrahydro-1,7-naphthyridine

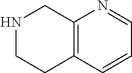

To a solution of 6,7-dihydro-1,7-naphthyridin-8(5H)-one (2.1 g, 14.2 mmol) in a mixture solution of tetrahydrofuran (300 mL) and dichloromethane (100 mL) was added BH₃.Me₂S (10 M, 14.2 mL, 142 mmol) dropwise at 0° C. After addition, the resulting mixture was heated to reflux and stirred until completion of the reaction. The mixture was cooled to −78° C. and quenched by addition of methanol (30 mL). The solution was stirred at ambient temperature and then HCl/methanol (20 mL) was added. The resulting mixture was stirred at ambient temperature for a further 16 h at which point the resulting mixture was concentrated in vacuo to give the crude product which was used in the next step without further purification (2.0 g). LCMS (m/z): 135.1 (M+1).

### Step 6: (R)-7-(oxiran-2-ylmethyl)-5,6,7,8-tetrahydro-1,7-naphthyridine

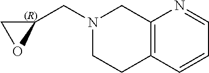

To a solution of 5,6,7,8-tetrahydro-1,7-naphthyridine (2.0 g, 14.9 mmol) in dimethylformamide (20 mL) was added triethylamine (1.5 g, 14.9 mmol), KF (3.4 g, 59.6 mmol) and (S)-oxiran-2-ylmethyl 3-nitrobenzenesulfonate (4.6 g, 17.9 mmol). The mixture was stirred at 30° C. for 16 h before being filtered. The filtrate was evaporated to give the crude product which was used in next step without further purification. LCMS (m/z): 191.1 (M+1).

### Step 7: (S)-1-amino-3-(5,6-dihydro-1,7-naphthyridin-7(8H)-yl)propan-2-ol

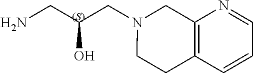

To a solution of (R)-7-(oxiran-2-ylmethyl)-5,6,7,8-tetrahydro-1,7-naphthyridine (2.7 g, 14.2 mmol) in dimethylformamide (20 mL) and ethanol (40 mL) was added ammonium hydroxide (100 mL). The mixture was stirred at 70° C. for 3 h. The reaction solution was concentrated, and the residue dissolved in methanol (30 mL) and filtered. The filtrate was concentrated to give the desired crude product (3.0 g) which was used in the next step without further purification. LCMS (m/z): 208.2 (M+1).

### Step 8: (S)-6-chloro-N-(3-(5,6-dihydro-1,7-naphthyridin-7(8H)-yl)-2-hydroxypropyl)pyrimidine-4-carboxamide

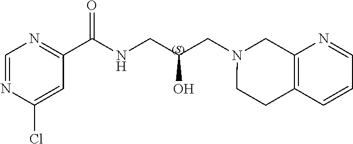

To a solution of (S)-1-amino-3-(5,6-dihydro-1,7-naphthyridin-7(8H)-yl)propan-2-ol (2.9 g, 14.2 mmol) in a solution of dichloromethane (40 mL) and dimethylformamide (6 mL) was added triethylamine (4.3 g, 42.6 mmol) followed by a solution of 6-chloropyrimidine-4-carbonyl chloride (3.0 g, 17.0 mmol) in dichloromethane (8 mL) at −20° C. Upon completion of the addition, the mixture was warmed to 30° C. and stirred for 30 min. The reaction mixture was diluted with water (30 mL) and extracted with dichloromethane (50 mL×2). The combined organic layers were concentrated in vacuo, and the residue was purified by flash chromatography on SiO₂ to give the desired product (1.5 g, 30.6%). LCMS (m/z): 348.1 (M+1).

### Step 9: (S)-6-((1-acetylpiperidin-4-yl)amino)-N-(3-(5,6-dihydro-1,7-naphthyridin-7(8H)-yl)-2-hydroxypropyl)pyrimidine-4-carboxamide (Compound 2-11)

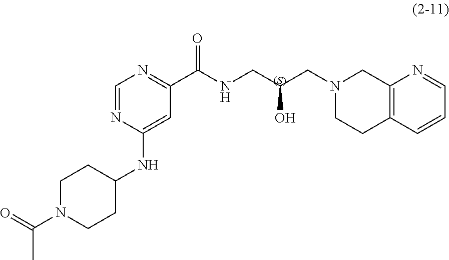

To a solution of (S)-6-chloro-N-(3-(5,6-dihydro-1,7-naphthyridin-7(8H)-yl)-2-hydro xylpropyl)pyrimidine-4-carboxamide (150 mg, 0.43 mmol) in 2-propanol (15 mL) were added N,N-diisopropylethylamine (166 mg, 1.29 mmol) and 1-(4-aminopiperidin-1-yl)ethanone (122 mg, 0.86 mmol). Following the addition, the mixture was stirred at 100° C. for 3 h, at which time LCMS analysis indicated completion of the reaction. The solvent was evaporated and the residue purified by preparative HPLC to give the TFA salt of the desired compound (62 mg, 32%) as a white solid. ¹H NMR (400 MHz, CD₃OD, δ): 8.58 (s, 1H), 8.53-8.44 (m, 1H), 7.79 (d, J=7.8 Hz, 1H), 7.42 (dd, J=4.8, 7.8 Hz, 1H), 7.20 (br. s., 1H), 4.59 (s, 2H), 4.49 (d, J=12.4 Hz, 1H), 4.44-4.25 (m, 2H), 3.98 (d, J=14.1 Hz, 1H), 3.73 (br. s, 2H), 3.60-3.40 (m, 4H), 3.29-3.22 (m, 2H), 2.94-2.86 (m, 1H), 2.20-1.97 (m, 6H), 1.60-1.43 (m, 2H). LCMS (m/z): 454.1 (M+1).

### Example 8: Preparation of (S)-6-((1-acetylpiperidin-4-yl)amino)-N-(3-(3,4-dihydro-2,7-naphthyridin-2(1H)-yl)-2-hydroxypropyl)pyrimidine-4-carboxamide (Compound 2-23)

### Step 1: 4-methylnicotinonitrile

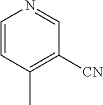

A mixture of 3-bromo-4-methylpyridine (9 g, 0.052 mol), Zn(CN)₂ (3.7 g, 0.031 mol), Pd₂(dba)₃ (2.4 g, 2.6 mmol), dppf (2.9 g, 5.2 mmol), and Zn (0.34 g, 0.052 mol) in dimethylformamide (100 mL) was stirred at 100° C. under a nitrogen atmosphere for 16 h. The mixture was filtered and the filtrate concentrated in vacuo. The residue was purified by flash chromatography on SiO₂ to give the desired product (5 g, 82%). LCMS (m/z): 119.1 (M+1).

### Step 2: ethyl 2-(3-cyanopyridin-4-yl)acetate

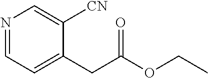

To a mixture of 4-methylnicotinonitrile (2.3 g, 19.5 mmol) and Et₂CO₃ (23 g, 195 mmol) was added sodium hydride (60% in mineral oil, 3.8 g, 97.5 mmol) at 0° C. Following the addition, the mixture was heated to reflux for 16 h, at which time LCMS indicated completion of the reaction. The resulting mixture was cooled to 0° C. and then quenched by addition of saturated aqueous ammonium chloride (50 mL). The aqueous layer was extracted with ethyl acetate (100 mL×3), the combined organic layers were dried over Na₂SO₄ and concentrated in vacuo, and the crude product purified by flash chromatography on SiO₂ to give the desired product (1.25 g, 34%). ¹H NMR (400 MHz, CDCl₃, δ): 8.89 (s, 1H), 8.77 (d, J=5.1 Hz, 1H), 7.43 (d, J=5.1 Hz, 1H), 4.24 (q, J=7.2 Hz, 2H), 3.90 (s, 2H), 1.34-1.28 (m, 3H). LCMS (m/z): 191.1 (M+1).

### Step 3: 1,2-dihydro-2,7-naphthyridin-3(4H)-one

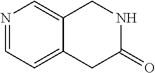 [SMILES: O=C1Cc2ccncc2CN1]

A mixture of ethyl 2-(3-cyanopyridin-4-yl)acetate (1.25 g, 6.6 mmol) and Raney Ni (1.2 g) in a solution of ethanol (20 mL) and water (20 mL) was hydrogenated (50 psi) at 50° C. for 16 h. After cooling, the mixture was filtered and the filtrate concentrated in vacuo to give the crude product (750 mg, 77%). ¹H NMR (400 MHz, CDCl₃): 8.58-8.45 (m, 2H), 7.16 (d, J=5.0 Hz, 1H), 6.25 (br. s., 1H), 4.60 (s, 2H), 3.63 (s, 2H). LCMS (m/z): 149.0 (M+1).

### Step 4: 1,2,3,4-tetrahydro-2,7-naphthyridine

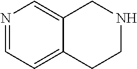

To a solution of 1,2-dihydro-2,7-naphthyridin-3(4H)-one (750 mg, 5.07 mmol) in a solution of tetrahydrofuran (300 mL) and dichloromethane (100 mL) was added BH₃.Me₂S (10 M, 5.1 mL, 51.0 mmol) dropwise at 0° C. Following the addition, the resulting mixture was stirred at reflux for 16 h, at which time LCMS indicated completion of the reaction. The mixture was cooled to −78° C. and the reaction was quenched by addition of methanol (10 mL). The solution was warmed to ambient temperature, whereupon HCl/methanol (20 mL) was added. The resulting mixture was stirred at ambient temperature for 16 h, then concentrated in vacuo to give the crude product (400 mg). LCMS (m/z): 135.1 (M+1).

### Step 5: (R)-2-(oxiran-2-ylmethyl)-1,2,3,4-tetrahydro-2,7-naphthyridine

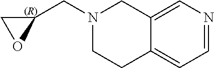

To a solution of 1,2,3,4-tetrahydro-2,7-naphthyridine (582 mg, 4.35 mmol) in dimethylformamide (20 mL) was added triethylamine (605 mg, 4.35 mmol), KF (1 g, 17.4 mmol), and (S)-oxiran-2-ylmethyl 3-nitrobenzenesulfonate (1.3 g, 4.35 mmol). The mixture was stirred at 30° C. for 16 h, then filtered. The solvent was evaporated to give the crude product which was used in the next step without further purification. LCMS (m/z): 191.1 (M+1).

### Step 6: (S)-1-amino-3-(3,4-dihydro-2,7-naphthyridin-2(1H)-yl)propan-2-ol

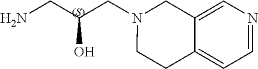

To a solution of (R)-2-(oxiran-2-ylmethyl)-1,2,3,4-tetrahydro-2,7-naphthyridine (826 mg, 4.35 mmol) in dimethylformamide (20 mL) and ethanol (20 mL) was added ammonium hydroxide (40 mL). The mixture was stirred at 70° C. for 3 h, then concentrated in vacuo. The residue was dissolved in methanol (30 mL) and filtered. The filtrate was then concentrated in vacuo to give the crude product (600 mg, 66.7%) which was used in next step without further purification. LCMS (m/z): 208.2 (M+1).

### Step 7: (S)-6-chloro-N-(3-(3,4-dihydro-2,7-naphthyridin-2(1H)-yl)-2-hydroxypropyl)pyrimidine-4-carboxamide

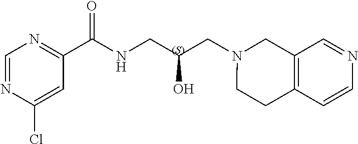

To a solution of (S)-1-amino-3-(3,4-dihydro-2,7-naphthyridin-2(1H)-yl)propan-2-ol (1.4 g, 6.72 mmol) in dichloromethane (40 mL) and DMSO (20 mL) was added triethylamine (2.8 mL, 20.25 mmol), followed by a solution of 6-chloropyrimidine-4-carbonyl chloride (1.2 g, 6.72 mmol) at −20° C. Following the addition, the mixture was stirred at 30° C. for 30 min and the reaction was quenched by addition of water (30 mL). The aqueous layer was extracted with dichloromethane (30 mL×3), and the combined organic layers were washed with brine (30 mL×3), dried over Na₂SO₄, and concentrated in vacuo. The crude product was purified by flash chromatography on SiO₂ to give the desired product (340 mg, 28%). LCMS (m/z): 348.1 (M+1).

### Step 8: (S)-6-((1-acetylpiperidin-4-yl)amino)-N-(3-(3,4-dihydro-2,7-naphthyridin-2(1H)-yl)-2-hydroxypropyl)pyrimidine-4-carboxamide (Compound 2-23)

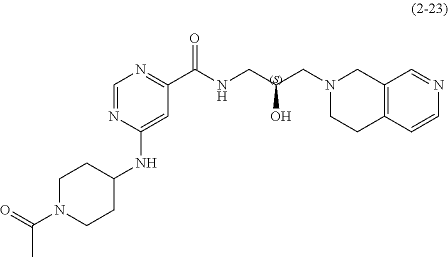

To a solution of (S)-6-chloro-N-(3-(3,4-dihydro-2,7-naphthyridin-2(1H)-yl-2-hydroxy propyl)pyrimidine-4-carboxamide (120 mg, 0.345 mmol) in 2-propanol (15 mL) was added N,N-diisopropylethylamine (145 mg, 1.04 mmol) and 1-(4-aminopiperidin-1-yl)ethanone (123 mg, 0.69 mmol). The resulting mixture was stirred at 100° C. for 3 h, following which time the solvent was removed in vacuo. The residue was purified by preparative HPLC to give the title compound (50 mg, 32%). ¹H NMR (400 MHz, CD₃OD, δ): 8.34-8.15 (m, 3H), 7.22 (d, J=5.1 Hz, 1H), 7.10 (s, 1H), 4.45 (d, J=13.4 Hz, 1H), 4.27-4.02 (m, 2H), 3.95 (d, J=15.3 Hz, 1H), 3.78 (s, 2H), 3.60-3.50 (m, 2H), 3.32-3.25 (m, 1H), 3.05-2.96 (m, 2H), 2.93-2.85 (m, 3H), 2.71 (d, J=6.3 Hz, 2H), 2.18-2.00 (m, 5H), 1.53-1.38 (m, 2H). LCMS (m/z): 454.3 (M+1).

### Example 9: Preparation of (S)-6-((1-acetylpiperidin-4-yl)amino)-N-(3-(3,4-dihydro-2,6-naphthyridin-2(1H)-yl)-2-hydroxypropyl)pyrimidine-4-carboxamide (Compound 2-17)

### Step 1: 3-bromoisonicotinaldehyde

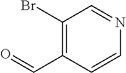

To a solution of diisopropylamine (27.6 g, 273.0 mmol) in tetrahydrofuran (300 mL) was added dropwise a solution of n-butyllithium (91 mL, 228.0 mmol, 2.5 M) in tetrahydrofuran at −78° C. under nitrogen. The mixture was stirred at −78° C. for 1 h, followed by the dropwise addition of 3-bromopyridine (30.0 g, 190.0 mmol). The mixture was stirred at −78° C. for 20 min before dimethylformamide (55.0 g, 760.0 mmol) was added dropwise. After addition, the reaction mixture was stirred at −78° C. for 1 h and was then warmed to ambient temperature before being quenched by addition of saturated aqueous ammonium chloride (150 mL). The aqueous layer was extracted with ethyl acetate (200 mL×3), the combined organic layers were concentrated in vacuo, and the resulting residue was purified by flash chromatography on SiO₂ to give the desired product (12.0 g, 34.0%). ¹H NMR (400 MHz, CD₃OD, δ): 10.37 (s, 1H), 8.91 (s, 1H), 8.80-8.61 (m, 1H), 7.78-7.63 (m, 1H). LCMS (m/z): 187.0 (M+1).

### Step 2: 3-((trimethylsilyl)ethynyl)isonicotinaldehyde

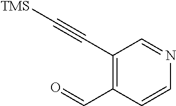 [SMILES: C[Si](C)(C)C#Cc1cnccc1C=O]

A mixture of 3-bromoisonicotinaldehyde (12.0 g, 64.5 mmol), ethynyltrimethylsilane (18.9 g, 193.5 mmol), Pd(dppf)Cl₂ (1.0 g), CuI (500 mg), and triethylamine (9.8 g, 96.7 mmol) in dimethylformamide (60 mL) was stirred at 80° C. for 2 h under nitrogen. The reaction was diluted with water (100 mL) and the aqueous layer was extracted with ethyl acetate (200 mL×3). The combined organic layers were dried and concentrated in vacuo. The resulting residue was purified by flash chromatography to give the desired product (6.0 g, 46.2%). LCMS (m/z): 204.0 (M+1).

### Step 3: 2,6-naphthyridine

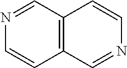

A mixture of 3-((trimethylsilyl)ethynyl)isonicotinaldehyde (6.0 g, 29.5 mmol) and liquid ammonia (60 mL) in ethanol (300 mL) was heated at 80° C. in a sealed tube for 16 h. After cooling to ambient temperature, the reaction mixture was filtered, and the filtrate was concentrated in vacuo. The residue was purified by flash chromatography on SiO₂ to give the desired product (800 mg, 20.9%). ¹H NMR (400 MHz, CD₃OD, δ): 9.39 (s, 2H), 8.69 (d, J=5.8 Hz, 2H), 8.02 (d, J=5.8 Hz, 2H). LCMS (m/z): 131.0 (M+1).

### Step 4: 1,2,3,4-tetrahydro-2,6-naphthyridine

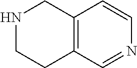

A mixture of 2,6-naphthyridine (800 mg, 6.15 mmol), CaO (413 mg, 7.38 mmol), and PtO₂ (200 mg) in CH₃OCH₂CH₂OH (80 mL) was hydrogenated (50 psi) at 30° C. for 16 h. After the reaction was complete, the mixture was filtered, and the filtrate was concentrated in vacuo to give the crude product (860 mg). ¹H NMR (400 MHz, CD₃OD, δ): 8.42-8.38 (m, 1H), 8.37-8.31 (m, 1H), 7.31-7.21 (m, 1H), 4.37-4.23 (m, 2H), 3.49-3.37 (m, 2H), 3.13-3.03 (m, 2H). LCMS (m/z): 135.0 (M+1).

### Step 5: (R)-2-(oxiran-2-ylmethyl)-1,2,3,4-tetrahydro-2,6-naphthyridine

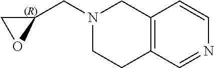

To a solution of 1,2,3,4-tetrahydro-2,6-naphthyridine (1.03 g, 7.7 mmol) in dimethylformamide (20 mL) was added KF (1.8 g, 30.8 mmol) and (S)-oxiran-2-ylmethyl 3-nitro benzenesulfonate (2.4 g, 9.24 mmol). The mixture was stirred at 30° C. for 16 h, whereupon the solvent was evaporated to give the crude product, which was used in the next step without further purification. LCMS (m/z): 191.1 (M+1).

### Step 6: (S)-1-amino-3-(3,4-dihydro-2,6-naphthyridin-2(1H)-yl)propan-2-ol

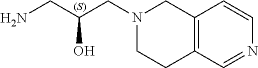

To a solution of (R)-2-(oxiran-2-ylmethyl)-1,2,3,4-tetrahydro-2,6-naphthyridine (1.46 g, 7.7 mmol) in dimethylformamide (20 mL) and ethanol (30 mL) was added ammonium hydroxide (50 mL). The reaction was stirred at 100° C. for 3 h, whereupon the solution was concentrated, and the resulting residue was dissolved in methanol (25 mL) and filtered. The filtrate was concentrated in vacuo to give the crude product (2.0 g) which was used in next step without further purification. LCMS (m/z): 208.2 (M+1).

### Step 7: (S)-6-chloro-N-(3-(3,4-dihydro-2,6-naphthyridin-2(1H)-yl)-2-hydroxypropyl)pyrimidine-4-carboxamide

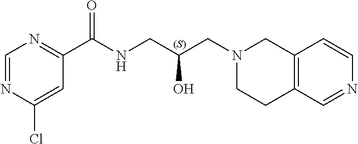

To a solution of (S)-1-amino-3-(3,4-dihydro-2,6-naphthyridin-2(1H)-yl)propan-2-ol (1.59 g, 7.7 mmol) in dichloromethane (10 mL) and dimethylformamide (6 mL) was added triethylamine (1.56 g, 15.4 mmol) followed by a solution of 6-chloropyrimidine-4-carbonyl chloride (1.54 g, 9.24 mmol) in dichloromethane (6 mL) at 0° C. Following the addition, the mixture was stirred at 30° C. for 30 min and then diluted with water (30 mL). The aqueous layer was extracted with dichloromethane (30 mL×2), the combined organic layers were dried and concentrated in vacuo. The resulting residue was purified by flash chromatography on SiO₂ to give the desired product (700 mg, 26.2%). LCMS (m/z): 348.2 (M+1).

### Step 8: (S)-6-((1-acetylpiperidin-4-yl)amino)-N-(3-(3,4-dihydro-2,6-naphthyridin-2(1H)-yl)-2-hydroxypropyl)pyrimidine-4-carboxamide (Compound 2-17)

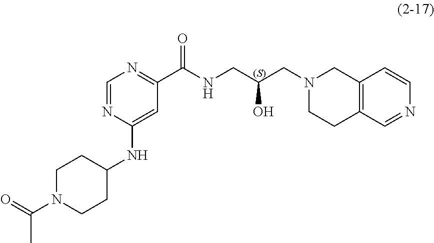

To a solution of (S)-6-chloro-N-(3-(3,4-dihydro-2,6-naphthyridin-2(1H)-yl)-2-hydroxypropyl)pyrimidine-4-carboxamide (150 mg, 0.43 mmol) in 2-propanol (15 mL) was added N,N-diisopropylethylamine (166 mg, 1.29 mmol) and 1-(4-aminopiperidin-1-yl)ethanone (92 mg, 0.65 mmol). The reaction was stirred at 100° C. for 3 h, following which the solvent was removed in vacuo. The resulting residue was purified by preparative HPLC to give the TFA salt of the title compound (30 mg, 15.4%). ¹H NMR (400 MHz, CD₃OD, δ): 8.84-8.72 (m, 1H), 8.71-8.48 (m, 2H), 7.86-7.66 (m, 1H), 7.32-7.07 (m, 1H), 4.81-4.71 (m, 2H), 4.55-4.24 (m, 3H), 4.01-3.91 (m, 1H), 3.84-3.68 (m, 2H), 3.65-3.32 (m, 7H), 2.94-2.79 (m, 1H), 2.13 (s, 5H), 1.66-1.39 (m, 2H). LCMS (m/z): 454.2 (M+1).

### Example 10: Preparation of (S)—N-(3-(7,8-dihydro-1,6-naphthyridin-6(5H)-yl)-2-hydroxypropyl)-6-(oxetan-3-ylamino)pyrimidine-4-carboxamide (Compound 2-12)

### Step 1: methyl 2-methylnicotinate

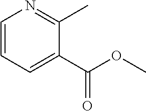

A mixture of 3-bromo-2-methylpyridine (5.0 g, 29.0 mmol), Pd(dppf)Cl₂ (2.1 g, 2.9 mmol), and triethylamine (8.8 g, 87 mmol) in methanol (250 mL) was stirred at 80° C. under a CO atmosphere (50 psi) for 16 h. After cooling, the reaction solution was filtered and the filtrate concentrated in vacuo. The resulting residue was purified by flash chromatography on SiO₂ to give the desired product (4.1 g, 93.6%). ¹H-NMR (400 MHz, CD₃OD, δ): 8.57-8.42 (m, 1H), 8.16-8.00 (m, 1H), 7.17-7.02 (m, 1H), 3.81 (s, 3H), 2.73 (s, 3H). LCMS (m/z): 152.0 (M+1).

### Step 2: methyl 2-(bromomethyl)nicotinate

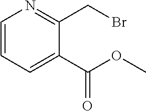

A mixture of methyl 2-methylnicotinate (4.1 g, 27.1 mmol), NBS (5.8 g, 32.5 mmol), AIBN (100 mg, 0.61 mmol) in carbon tetrachloride (55 mL) was stirred at 90° C. for 16 h under nitrogen. Once cooled, the reaction mixture was diluted with water (25 mL) and the aqueous layer was extracted with dichloromethane (50 mL×3). The combined organic layers were concentrated in vacuo, and the resulting residue was purified by flash chromatography on SiO₂ to give the desired product (5.0 g, 80.6%). LCMS (m/z): 229.9 (M+1).

### Step 3: methyl 2-(cyanomethyl)nicotinate

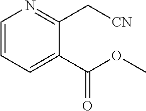

To a solution of methyl 2-(bromomethyl)nicotinate (6.0 g, 26.0 mmol) in acetonitrile (200 mL) was added TBAF (10.2 g, 39.0 mmol) and TMSCN (5.2 g, 52.0 mmol) at 0° C. Following the addition, the reaction was stirred at ambient temperature for 16 h and then diluted with water (30 mL). The aqueous layer was extracted with ethyl acetate (50 mL×3) and the combined organic layers were concentrated. The resulting residue was purified by flash chromatography on SiO₂ to give the desired product (2.3 g, 50.3%). LCMS (m/z): 177.0 (M+1).

### Step 4: 7,8-dihydro-1,6-naphthyridin-5(6H)-one

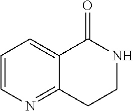 [SMILES: O=C1NCCc2ncccc21]

A mixture of methyl 2-(cyanomethyl)nicotinate (2.3 g, 13.0 mmol) and Raney Ni (400 mg) in methanol (40 mL) and water (40 mL) was hydrogenated (50 psi) at 50° C. for 16 h. The reaction mixture was filtered and the filtrate was concentrated in vacuo to give the crude product (2.1 g). This crude was used in the next step without further purification. LCMS (m/z): 149.0 (M+1).

### Step 5: 5,6,7,8-tetrahydro-1,6-naphthyridine

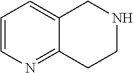

To a solution of 7,8-dihydro-1,6-naphthyridin-5(6H)-one (2.1 g, 14.2 mmol) in tetrahydrofuran (300 mL) and dichloromethane (100 mL) was added BH₃.Me₂S (10 M, 14.2 mL, 142.0 mmol) dropwise at 0° C. Following the addition, the reaction was stirred at reflux for 16 h until completion of the reaction. The reaction was then cooled to −78° C. and quenched by addition of methanol (25 mL). The solution was warmed to ambient temperature, whereupon HCl/methanol (20 mL) was added. The resulting mixture was stirred at 90° C. for another 3 h. The mixture was concentrated in vacuo to give the crude product (2.0 g). LCMS (m/z): 135.1 (M+1).

### Step 6: (R)-6-(oxiran-2-ylmethyl)-5,6,7,8-tetrahydro-1,6-naphthyridine

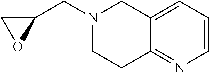 [SMILES: c1cnc2c(c1)CN(C[C@@H]1CO1)CC2]

To a solution of 5,6,7,8-tetrahydro-1,6-naphthyridine (2.0 g, 14.9 mmol) in dimethylformamide (20 mL) was added triethylamine (1.5 g, 14.9 mmol), KF (3.4 g, 59.6 mmol), and (S)-oxiran-2-ylmethyl 3-nitrobenzenesulfonate (4.6 g, 17.9 mmol). The mixture was stirred at 30° C. under nitrogen for 16 h, then evaporated to give the crude product which was used in next step without further purification. LCMS (m/z): 191.1 (M+1).

### Step 7: (S)-1-amino-3-(7,8-dihydro-1,6-naphthyridin-6(5H)-yl)propan-2-ol

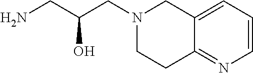 [SMILES: NC[C@H](O)CN1CCc2ncccc2C1]

To a solution of (R)-6-(oxiran-2-ylmethyl)-5,6,7,8-tetrahydro-1,6-naphthyridine (2.7 g, 14.2 mmol) in dimethylformamide (20 mL) and ethanol (40 mL) was added ammonium hydroxide (100 mL). The mixture was stirred at 100° C. for 3 h before being cooled and concentrated in vacuo. The residue was then dissolved in methanol (30 mL) and filtered. The filtrate was concentrated in vacuo to give the crude product (3.0 g), which was used in next step without further purification. LCMS (m/z): 208.2 (M+1).

### Step 8: (S)-6-chloro-N-(3-(7,8-dihydro-1,6-naphthyridin-6(5H)-yl)-2-hydroxypropyl)pyrimidine-4-carboxamide

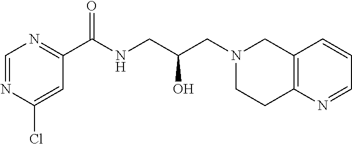 [SMILES: O=C(NC[C@H](O)CN1CCc2ncccc2C1)c1cc(Cl)ncn1]

To a solution of (S)-1-amino-3-(7,8-dihydro-1,6-naphthyridin-6(5H)-yl)propan-2-ol (2.9 g, 14.2 mmol) in dichloromethane (40 mL) and dimethylformamide (6 mL) was added triethylamine (4.3 g, 42.6 mmol) and a solution of 6-chloropyrimidine-4-carbonyl chloride (3.0 g, 17.0 mmol) in dichloromethane (8 mL) at 0° C. Following the addition, the mixture was stirred at ambient temperature for 30 min and then diluted with water (30 mL). The aqueous layer was then extracted with dichloromethane (50 mL×3) and the combined organic layers were concentrated in vacuo. The residue was purified by flash chromatography on SiO₂ to give the desired product (1.5 g, 30.6%). LCMS (m/z): 348.1 (M+1).

### Step 9: (S)—N-(3-(7,8-dihydro-1,6-naphthyridin-6(5H)-yl)-2-hydroxypropyl)-6-(oxetan-3-ylamino)pyrimidine-4-carboxamide (Compound 2-12)

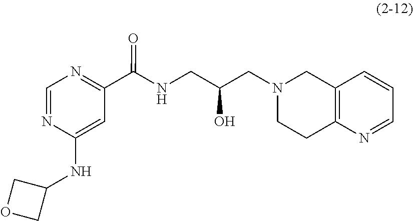 [SMILES: O=C(NC[C@H](O)CN1CCc2ncccc2C1)c1cc(NC2COC2)ncn1]

To a solution of (S)-6-chloro-N-(3-(7,8-dihydro-1,6-naphthyridin-6(5H)-yl)-2-hydro xylpropyl)pyrimidine-4-carboxamide (150 mg, 0.43 mmol) in 2-propanol (15 mL) was added N,N-diisopropylethylamine (166 mg, 1.29 mmol) and oxetan-3-amine (63 mg, 0.86 mmol). The reaction was stirred at 100° C. for 3 h, whereupon the solvents were removed in vacuo. The resulting residue was purified by preparative HPLC to give the title compound (80 mg, 48.5%). ¹H-NMR (400 MHz, CD₃OD, δ): 8.36-8.25 (m, 1H), 8.25-8.16 (m, 1H), 7.53-7.44 (m, 1H), 7.23-7.15 (m, 1H), 7.14-7.04 (m, 1H), 5.17-5.01 (m, 1H), 4.97-4.92 (m, 2H), 4.59 (s, 2H), 4.13-3.98 (m, 1H), 3.75 (s, 2H), 3.60-3.45 (m, 2H), 3.08-2.87 (m, 4H), 2.76-2.59 (m, 2H). LCMS (m/z): 385.1 (M+1).

### Example 11: Preparation of (S)-6-((1-acetylazetidin-3-yl)amino)-N-(3-(7,8-dihydro-1,6-naphthyridin-6(5H)-yl)-2-hydroxypropyl)pyrimidine-4-carboxamide (Compound 2-9)

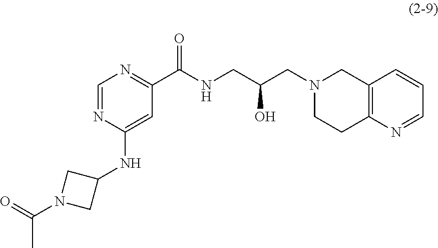 [SMILES: CC(=O)N1CC(Nc2cc(C(=O)NC[C@H](O)CN3CCc4ncccc4C3)ncn2)C1]

To a solution of (S)-6-chloro-N-(3-(7,8-dihydro-1,6-naphthyridin-6(5H)-yl)-2-hydroxypropyl)pyrimidine-4-carboxamide (150 mg, 0.43 mmol) in 2-propanol (15 mL) was added N,N-diisopropylethylamine (166 mg, 1.29 mmol) and 1-(3-aminoazetidin-1-yl)ethanone (98 mg, 0.86 mmol). The mixture was stirred at 100° C. for 3 h, whereupon the solvent was removed in vacuo. The resulting residue was purified by preparative HPLC to give the title compound as the TFA salt (30 mg, 16.4%). ¹H-NMR (400 MHz, CD₃OD, δ): 8.68-8.51 (m, 2H), 8.06-7.93 (m, 1H), 7.66-7.55 (m, 1H), 7.31-7.18 (m, 1H), 4.86-4.75 (m, 1H), 4.74-4.65 (m, 2H), 4.64-4.54 (m, 1H), 4.45-4.30 (m, 2H), 4.19-4.07 (m, 1H), 3.98-3.88 (m, 1H), 3.87-3.76 (m, 2H), 3.63-3.51 (m, 2H), 3.49-3.33 (m, 4H), 1.90 (s, 3H). LCMS (m/z): 426.2 (M+1).

### Example 12: Preparation of (S)-6-((1-acetylpiperidin-4-yl)amino)-N-(3-(7,8-dihydro-1,6-naphthyridin-6(5H)-yl)-2-hydroxypropyl)pyrimidine-4-carboxamide (Compound 2-14)

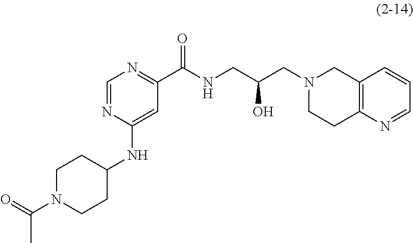 [SMILES: CC(=O)N1CCC(Nc2cc(C(=O)NC[C@H](O)CN3CCc4ncccc4C3)ncn2)CC1]

To a solution of (S)-6-chloro-N-(3-(7,8-dihydro-1,6-naphthyridin-6(5H)-yl)-2-hydroxypropyl)pyrimidine-4-carboxamide (150 mg, 0.43 mmol) in 2-propanol (15 mL) was added N,N-diisopropylethylamine (166 mg, 1.29 mmol) and 1-(4-aminopiperidin-1-yl)ethanone (122 mg, 0.86 mmol). The mixture was stirred at 100° C. for 3 h, whereupon the solvent was removed in vacuo. The resulting residue was purified by preparative HPLC to give the title compound as the TFA salt (83 mg, 41.0%). ¹H-NMR (400 MHz, CD₃OD, δ): 8.70-8.53 (m, 2H), 8.11-7.95 (m, 1H), 7.72-7.56 (m, 1H), 7.34-7.14 (m, 1H), 4.78-4.62 (m, 2H), 4.59-4.24 (m, 3H), 4.05-3.92 (m, 1H), 3.90-3.76 (m, 2H), 3.62-3.31 (m, 7H), 2.96-2.80 (m, 1H), 2.21-1.98 (m, 5H), 1.68-1.42 (m, 2H). LCMS (m/z): 454.2 (M+1).

### Example 13: Preparation of (S)—N-(2-hydroxy-3-(3-phenyl-6,7-dihydro-1H-pyrazolo[4,3-c]pyridin-5(4H)-yl)propyl)-6-(oxetan-3-ylamino)pyrimidine-4-carboxamide (Compound 3-17)

### Step 1: tert-butyl 3-benzoyl-4-oxopiperidine-1-carboxylate

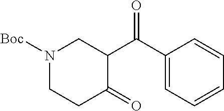 [SMILES: CC(C)(C)OC(=O)N1CCC(=O)C(C(=O)c2ccccc2)C1]

To a cooled solution (−30° C.) of tert-butyl 4-oxopiperidine-1-carboxylate (4.0 g, 20 mmol) in tetrahydrofuran (50 mL) was added LiHMDS (20 mL, IM) over 15 min. After stirring for a further 10 min at this temperature, benzyl chloride (2.8 g, 20 mmol) in 10 mL tetrahydrofuran was then added carefully. The mixture was then stirred for another 3 h at −30° C. before addition of aq. ammonium chloride to quench the reaction. The solution was then diluted with ethyl acetate and the organic layer washed with water, dried over anhydrous Na₂SO₄, filtered, and evaporated to yield the crude product (5.5 g, 90%) which used directly in the next step. LCMS (m/z): 304.1 (M+1).

### Step 2: tert-butyl 3-phenyl-6,7-dihydro-1H-pyrazolo[4,3-c]pyridine-5(4H)-carboxylate

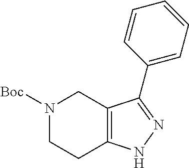 [SMILES: CC(C)(C)OC(=O)N1CCc2[nH]nc(-c3ccccc3)c2C1]

To a solution of tert-butyl 3-benzoyl-4-oxopiperidine-1-carboxylate (5.5 g, 18 mmol) in ethanol (50 mL) was added hydrazine (80%, 5 mL, 80 mmol) at 20° C. After stirring for 30 min, the solvent was evaporated and the resulting residue then dissolved in ethyl acetate (50 mL), washed with brine, dried over anhydrous Na₂SO₄, and filtered. The filtrate was evaporated and the resulting residue purified by flash chromatography on SiO₂ to afford the desired product (5.0 g, 92%) as a pale yellow oil. LCMS (m/z): 300.1 (M+1).

### Step 3: 1-benzyl 5-tert-butyl 3-phenyl-6,7-dihydro-1H-pyrazolo[4,3-c]pyridine-1,5(4H)-dicarboxylate

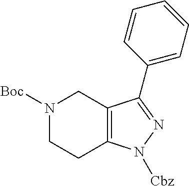 [SMILES: CC(C)(C)OC(=O)N1CCc2c(c(-c3ccccc3)nn2C(=O)OCc2ccccc2)C1]

To a solution of tert-butyl 3-phenyl-6,7-dihydro-1H-pyrazolo[4,3-c]pyridine-5(4H)-carboxylate (4.5 g, 15 mmol) and triethylamine (4.5 g, 45 mmol) in dichloromethane (100 mL) was added Cbz-Cl (3.0 g, 18 mmol) at 0° C. The mixture was then stirred at ambient temperature for 12 h before evaporation of the solvent. The resulting crude product was purified by flash chromatography on SiO₂ to give the desired product (3.1 g, 47%). LCMS (m/z): 434.2 (M+1).

### Step 4: benzyl 3-phenyl-4,5,6,7-tetrahydro-1H-pyrazolo[4,3-c]pyridine-1-carboxylate

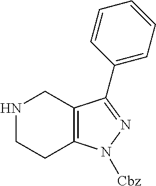 [SMILES: O=C(OCc1ccccc1)n1nc(-c2ccccc2)c2c1CCNC2]

To a solution of 1-benzyl 5-tert-butyl 3-phenyl-6,7-dihydro-1H-pyrazolo[4,3-c]pyridine-1,5(4H)-dicarboxylate (1.5 g, 3.5 mmol) in ethyl acetate (30 mL) was added 4N HCl/ethyl acetate (10 mL) and the combined mixture was stirred at ambient temperature for 12 h. The reaction mixture was then concentrated in vacuo to give the crude product (900 mg, 81%), which was used in the next step without further purification. LCMS (m/z): 334.2 (M+1).

### Step 5: (R)-benzyl 5-(oxiran-2-ylmethyl)-3-phenyl-4,5,6,7-tetrahydro-1H-pyrazolo[4,3-c]pyridine-1-carboxylate

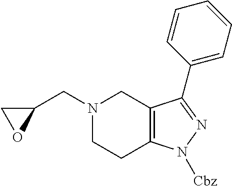 [SMILES: O=C(OCc1ccccc1)n1nc(-c2ccccc2)c2c1CCN(C[C@@H]1CO1)C2]

A mixture of benzyl 3-phenyl-4,5,6,7-tetrahydro-H-pyrazolo[4,3-c]pyridine-1-carboxylate (900 mg, 2.4 mmol), (S)-oxiran-2-ylmethyl 3-nitrobenzenesulfonate (751 mg, 2.9 mmol), triethylamine (970 mg, 9.6 mmol), and KF (557 mg, 9.6 mmol) in tetrahydrofuran (50 mL) was stirred at 25° C. for 16 h until completion of the reaction. The mixture was filtered and the filtrate concentrated in vacuo to give the crude product (1.1 g, 118%) which was used in the next step without further purification. LCMS (m/z): 390.2 (M+1).

### Step 6: (S)-benzyl 5-(3-amino-2-hydroxypropyl)-3-phenyl-4,5,6,7-tetrahydro-1H-pyrazolo[4,3-c]pyridine-1-carboxylate

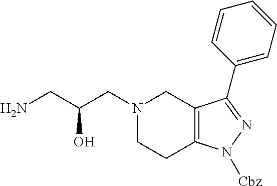 [SMILES: NC[C@H](O)CN1CCc2c(c(-c3ccccc3)nn2C(=O)OCc2ccccc2)C1]

A solution of (R)-benzyl 5-(oxiran-2-ylmethyl)-3-phenyl-4,5,6,7-tetrahydro-H-pyrazolo[4,3-c]pyridine-1-carboxylate (900 mg, 2.3 mmol) in NH₃/ethanol (2 N, 100 mL) was heated at 80° C. in a sealed tube for 4 h. The reaction was cooled to ambient temperature and the solvent was removed in vacuo to give the crude product (1 g, 107%), which was used in next step without further purification. LCMS (m/z): 407.2 (M+1).

### Step 7: (S)-1-amino-3-(3-phenyl-6,7-dihydro-1H-pyrazolo[4,3-c]pyridin-5(4H)-yl)propan-2-ol

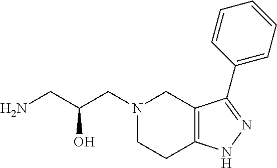 [SMILES: NC[C@H](O)CN1CCc2[nH]nc(-c3ccccc3)c2C1]

To a solution of (S)-benzyl 5-(3-amino-2-hydroxypropyl)-3-phenyl-4,5,6,7-tetrahydro-1H-pyrazolo[4,3-c]pyridine-1-carboxylate (1 g, 2.5 mmol) in methanol (50 mL) was added Pd/C (100 mg) under a nitrogen atmosphere and the mixture was stirred under a hydrogen atmosphere (50 psi) at 25° C. for 16 h. After the reaction, the catalyst was filtered off and the filtrate was concentrated in vacuo to give the crude product (800 mg, 117%). LCMS (m/z): 273.1 (M+1).

### Step 8: (S)-6-chloro-N-(2-hydroxy-3-(3-phenyl-6,7-dihydro-1H-pyrazolo[4,3-c]pyridin-5(4H)-yl)propyl)pyrimidine-4-carboxamide

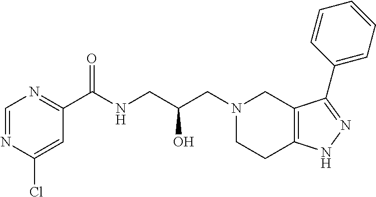 [SMILES: O=C(NC[C@H](O)CN1CCc2[nH]nc(-c3ccccc3)c2C1)c1cc(Cl)ncn1]

A mixture of (S)-1-amino-3-(3-phenyl-6,7-dihydro-1H-pyrazolo[4,3-c]pyridin-5(4H)-yl)propan-2-ol (800 mg, 2.9 mmol), 6-chloropyrimidine-4-carbonyl chloride (510 mg, 2.9 mmol), and triethylamine (1 mL) in dichloromethane (50 mL) was stirred at ambient temperature for 4 h. The mixture was then poured into 50 mL of ice-water, the aqueous layer was extracted with dichloromethane (50 mL×3), and the combined organic layers were dried (Na₂SO₄), filtered, and evaporated. The crude material was purified by preparative TLC to give the desired product (400 mg, 36%). LCMS (m/z): 413.1 (M+1).

### Step 9: (S)—N-(2-hydroxy-3-(3-phenyl-6,7-dihydro-1H-pyrazolo[4,3-c]pyridin-5(4H)-yl)propyl)-6-(oxetan-3-ylamino)pyrimidine-4-carboxamide (Compound 3-17)

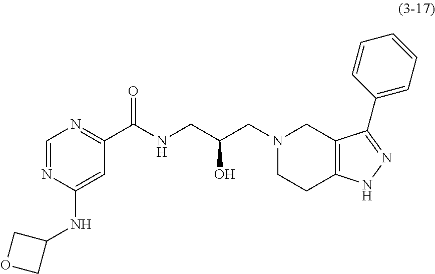 [SMILES: O=C(NC[C@H](O)CN1CCc2[nH]nc(-c3ccccc3)c2C1)c1cc(NC2COC2)ncn1]

A mixture of (S)-6-chloro-N-(2-hydroxy-3-(3-phenyl-6,7-dihydro-1H-pyrazolo[4,3-c]pyridin-5(4H)-yl)propyl)pyrimidine-4-carboxamide (100 mg, 0.24 mmol), oxetan-3-amine (21 mg, 0.29 mmol), and triethylamine (0.1 mL) in 2-propanol (10 mL) was stirred at 80° C. for 12 h. The solvent was evaporated and the resulting residue was purified by preparative HPLC to give the title compound (39.8 mg, 37%) as white solid. ¹H-NMR (400 MHz, CD₃OD, δ): 8.33 (s, 1H), 7.64-7.49 (m, 2H), 7.43 (s, 1H), 7.38-7.27 (m, 1H), 7.19-7.02 (m, 1H), 5.17-5.06 (m, 1H), 4.97 (t, J=6.78, 2 H), 4.61 (t, J=6.27 Hz, 2H), 4.12-4.02 (m, 1H), 3.80 (s, 2H), 3.53 (dd, J=5.52, 3.76 Hz, 2H), 3.02-2.92 (m, 2H), 2.88 (d, J=5.27 Hz, 2H), 2.76 (d, J=6.02 Hz, 2H). LCMS (m/z): 450.2 (M+1).

### Example 14: Preparation of (S)-6-((1-acetylazetidin-3-yl)amino)-N-(2-hydroxy-3-(3-phenyl-6,7-dihydro-1H-pyrazolo[4,3-c]pyridin-5(4H)-yl)propyl)pyrimidine-4-carboxamide (Compound 3-11)

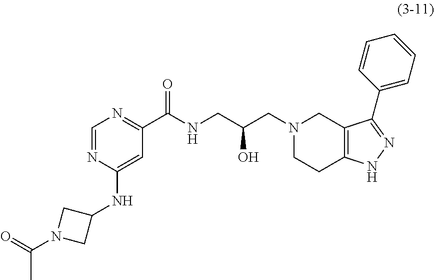 [SMILES: CC(=O)N1CC(Nc2cc(C(=O)NC[C@H](O)CN3CCc4[nH]nc(-c5ccccc5)c4C3)ncn2)C1]

A mixture of (S)-6-chloro-N-(2-hydroxy-3-(3-phenyl-6,7-dihydro-1H-pyrazolo[4,3-c]pyridin-5(4H)-yl)propyl)pyrimidine-4-carboxamide (100 mg, 0.24 mmol), 1-(3-aminoazetidin-1-yl) ethanone (33 mg, 0.29 mmol), and triethylamine (0.1 mL) in 2-propanol (10 mL) was stirred at 80° C. for 12 h. The solvent was evaporated and the resulting residue was purified by preparative HPLC to give the title compound (51.6 mg, 43%) as a white solid. ¹H-NMR (400 MHz, CD₃OD, δ): 8.43-8.30 (m, 1H), 7.64-7.51 (m, 2H), 7.49-7.39 (m, 2H), 7.38-7.29 (m, 1H), 7.18-7.06 (m, 1H), 4.80-4.68 (m, 1H), 4.62-4.54 (m, 1H), 4.39-4.31 (m, 1H), 4.01 (s, 2H), 3.94-3.86 (m, 1H), 3.80 (s, 2H), 3.58-3.47 (m, 2H), 3.03-2.92 (m, 2H), 2.87 (s, 2H), 2.76 (d, J=6.02 Hz, 2H), 1.91 (s, 3H). LCMS (m/z): 491.2 (M+1).

### Example 15: Preparation of (S)-6-((1-acetylpiperidin-4-yl)amino)-N-(2-hydroxy-3-(3-phenyl-6,7-dihydro-1H-pyrazolo[4,3-c]pyridin-5(4H)-yl)propyl)pyrimidine-4-carboxamide (Compound 3-12)

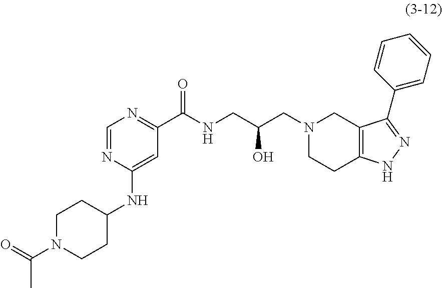 [SMILES: CC(=O)N1CCC(Nc2cc(C(=O)NC[C@H](O)CN3CCc4[nH]nc(-c5ccccc5)c4C3)ncn2)CC1]

A mixture of (S)-6-chloro-N-(2-hydroxy-3-(3-phenyl-6,7-dihydro-1H-pyrazolo[4,3-c]pyridin-5(4H)-yl)propyl)pyrimidine-4-carboxamide (100 mg, 0.24 mmol), 1-(4-aminopiperidin-1-yl) ethanone (45 mg, 0.32 mmol), and triethylamine (0.1 mL) in 2-propanol (10 mL) was stirred at 80° C. for 12 h. The solvent was evaporated and the resulting residue was purified by preparative HPLC to give the TFA salt of the title compound (91 mg, 73%) as white solid. ¹H-NMR (400 MHz, CD₃OD, δ): 8.65-8.54 (m, 1H), 7.59-7.54 (m, 2H), 7.53-7.48 (m, 2H), 7.46-7.40 (m, 1H), 7.33-7.09 (m, 1H), 4.78-4.57 (m, 2H), 4.55-4.46 (m, 1H), 4.44-4.24 (m, 2H), 4.20-3.88 (m, 2H), 3.86-3.46 (m, 5H), 3.43-3.35 (m, 1H), 3.25-3.14 (m, 2H), 2.96-2.81 (m, 1H), 2.18-2.14 (m, 3H), 2.05 (s, 2H), 1.63-1.44 (m, 2H). LCMS (m/z): 519.2 (M+1).

### Example 16: Preparation of (S)—N-(3-(6,7-dihydrothieno[3,2-c]pyridin-5(4H)-yl)-2-hydroxypropyl)-6-(oxetan-3-ylamino)pyrimidine-4-carboxamide (Compound 1-1)

### Step 1: (R)-5-(oxiran-2-ylmethyl)-4,5,6,7-tetrahydrothieno[3,2-c]pyridine

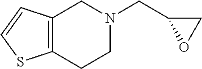 [SMILES: c1cc2c(s1)CCN(C[C@@H]1CO1)C2]

To a solution of 4,5,6,7-tetrahydrothieno[3,2-c]pyridine hydrochloride (4 g, 22.83 mmol) in tetrahydrofuran (100 mL) was added KF (4 g, 68.85 mmol), trimethylamine (3.5 g, 34.65 mmol), and (S)-oxiran-2-ylmethyl 3-nitrobenzenesulfonate (8 g, 30.86 mmol) at 0° C. Following the addition, the resulting mixture was stirred at ambient temperature for 16 h. Upon completion of the reaction, the mixture was filtered and the filtrate concentrated in vacuo to give the crude product (8 g) as a yellow oil that was used without further purification. LCMS (m/z): 196.1 (M+1).

### Step 2: (S)-1-amino-3-(6,7-dihydrothieno[3,2-c]pyridin-5(4H)-yl)propan-2-ol

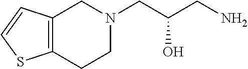 [SMILES: NC[C@H](O)CN1CCc2sccc2C1]

To a solution of (R)-5-(oxiran-2-ylmethyl)-4,5,6,7-tetrahydrothieno[3,2-c]pyridine (8 g crude, 22.83 mmol) in ethanol (50 mL) was added ammonium hydroxide (50 mL, 400 mmol). The resulting mixture was heated at reflux for 12 h. After cooling, the solvent was evaporated and the residue purified by flash chromatography to give the desired product (6 g, crude). LCMS (m/z): 213.1 (M+1).

### Step 3: (S)-6-chloro-N-(3-(6,7-dihydrothieno[3,2-c]pyridin-5(4H)-yl)-2-hydroxypropyl)pyrimidine-4-carboxamide

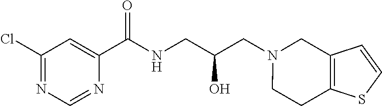 [SMILES: O=C(NC[C@H](O)CN1CCc2sccc2C1)c1cc(Cl)ncn1]

To a solution of (S)-1-amino-3-(6,7-dihydrothieno[3,2-c]pyridin-5(4H)-yl)propan-2-ol (6 g, 22.83 mmol) in dichloromethane (200 mL) was added triethylamine (10 g, 99 mmol) and 6-chloropyrimidine-4-carbonyl chloride (10 g, 56.5 mmoL) at 0° C. Following the addition, the reaction was stirred for 30 min and then quenched by addition of water (30 mL). The aqueous layer was extracted with dichloromethane (100 mL×3) and the combined organic layers were concentrated in vacuo. The resulting residue was purified by flash chromatography on SiO₂ to give the desired product (350 mg, 4.4%). LCMS (m/z): 353.1 (M+1).

### Step 4: (S)—N-(3-(6,7-dihydrothieno[3,2-c]pyridin-5(4H)-yl)-2-hydroxypropyl)-6-(oxetan-3-ylamino)pyrimidine-4-carboxamide (Compound 1-1)

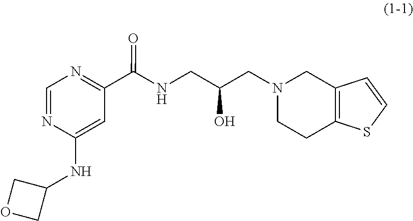 [SMILES: O=C(NC[C@H](O)CN1CCc2sccc2C1)c1cc(NC2COC2)ncn1]

To a solution of (S)-6-chloro-N-(3-(6,7-dihydrothieno[3,2-c]pyridin-5(4H)-yl)-2-hydroxypropyl) pyrimidine-4-carboxamide (100 mg, 0.28 mmol) in 2-propanol (10 mL) was added triethylamine (200 mg, 1.98 mmol) and oxetan-3-amine (30 mg, 0.41 mmol) at 30° C. The mixture was stirred at 100° C. for 3 h, cooled to ambient temperature, and concentrated in vacuo. The resulting residue was purified by preparative HPLC to give the title compound (57.9 mg, 52.6%) as a white solid. ¹H-NMR (400 MHz, CD₃OD, a): 8.33 (s, 1H), 7.13 (d, J=4.77 Hz, 2H), 6.72 (d, J=5.02 Hz, 1H), 5.09 (br. s, 1H), 4.95 (t, J=6.90 Hz, 2H), 4.60 (t, J=6.27 Hz, 2H), 4.04 (quin, J=5.96 Hz, 1H), 3.63 (s, 2H), 3.58-3.43 (m, 2H), 2.93-2.85 (m, 4H), 2.67 (d, J=6.27 Hz, 2H). LCMS (m/z): 390.2 (M+1).

### Example 17: Preparation of (S)-6-((1-acetylazetidin-3-yl)amino)-N-(3-(6,7-dihydrothieno[3,2-c]pyridin-5(4H)-yl)-2-hydroxypropyl)pyrimidine-4-carboxamide (Compound 1-2)

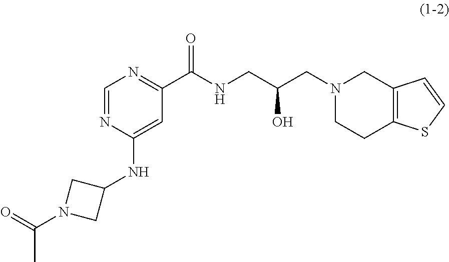 [SMILES: CC(=O)N1CC(Nc2cc(C(=O)NC[C@H](O)CN3CCc4sccc4C3)ncn2)C1]

To a solution of (S)-6-chloro-N-(3-(6,7-dihydrothieno[3,2-c]pyridin-5(4H)-yl)-2-hydroxypropyl) pyrimidine-4-carboxamide (100 mg, 0.28 mmol) in 2-propanol (10 mL) was added triethylamine (200 mg, 1.98 mmol) and 1-(3-aminoazetidin-1-yl)ethanone (60 mg, 0.42 mmol) at 30° C. The mixture was stirred at 100° C. for 3 h, cooled to ambient temperature, and concentrated in vacuo. The resulting residue was purified by preparative HPLC to give the title compound (26.2 mg, 20.4%) as a white solid. ¹H-NMR (400 MHz, CD₃OD, δ): 8.38 (s, 1H), 7.13 (d, J=5.27 Hz, 2H), 6.72 (d, J=5.02 Hz, 1H), 4.74 (br. s, 1H), 4.57 (t, J=8.41 Hz, 1H), 4.40-4.29 (m, 1H), 4.11-4.00 (m, 2H), 3.88 (dd, J=10.29, 5.27 Hz, 1H), 3.64 (s, 2H), 3.51 (qd, J=13.38, 5.77 Hz, 2H), 2.96-2.85 (m, 4H), 2.68 (d, J=6.02 Hz, 2H), 1.89 (s, 3H). LCMS (m/z): 431.2 (M+1).

### Example 18: Preparation of (S)-6-((1-acetylpiperidin-4-yl)amino)-N-(3-(6,7-dihydrothieno[3,2-c]pyridin-5(4H)-yl)-2-hydroxypropyl)pyrimidine-4-carboxamide (Compound 1-3)

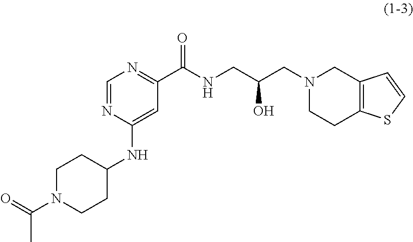 [SMILES: CC(=O)N1CCC(Nc2cc(C(=O)NC[C@H](O)CN3CCc4sccc4C3)ncn2)CC1]

To a solution of (S)-6-chloro-N-(3-(6,7-dihydrothieno[3,2-c]pyridin-5(4H)-yl)-2-hydroxypropyl) pyrimidine-4-carboxamide (150 mg, 0.28 mmol) in 2-propanol (10 mL) was added triethylamine (200 mg, 1.98 mmol) and 1-(4-aminopiperidin-1-yl)ethanone (100 mg, 0.88 mmol) at 30° C. The mixture was stirred at 100° C. for 3 h, cooled to ambient temperature, and concentrated in vacuo. The resulting residue was purified by preparative HPLC to give the title compound (33 mg, 17.8%) as a white solid. ¹H-NMR (400 MHz, CD₃OD, δ): 8.38-8.30 (m, 1H), 7.14 (d, J=5.14 Hz, 1H), 7.08 (s, 1H), 6.73 (d, J=5.15 Hz, 1H), 4.43 (d, J=12.42 Hz, 1H), 4.14 (br. s, 1H), 4.05 (quin, J=5.96 Hz, 1H), 3.93 (d, J=14.05 Hz, 1H), 3.65 (s, 2H), 3.58-3.43 (m, 2H), 3.30-3.24 (m, 1H), 2.95-2.87 (m, 5H), 2.68 (d, J=6.15 Hz, 2H), 2.12 (s, 3H), 2.10-1.97 (m, 2H), 1.55-1.36 (m, 2H). LCMS (m/z): 459.2 (M+1).

### Example 19: 1-(3-((cyclopentylamino)methyl)phenoxy)-3-(3,4-dihydro-2,7-naphthyridin-2(1H)-yl)propan-2-ol (Compound 2-2)

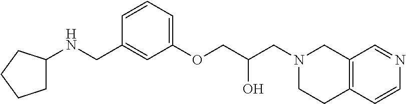 [SMILES: OC(COc1cccc(CNC2CCCC2)c1)CN1CCc2ccncc2C1]

### Step 1: tert-butyl cyclopentyl(3-hydroxybenzyl)carbamate

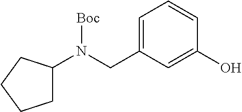 [SMILES: CC(C)(C)OC(=O)N(Cc1cccc(O)c1)C1CCCC1]

To a stirring solution of 3-hydroxybenzaldehyde (2.0 g, 16.4 mmol) and cyclopentanamine (1.4 g, 16.4 mmol) in MeOH (50 ml) was added AcOH (0.1 mL) and the reaction mixture stirred at 20° C. for 30 min. NaCNBH₃ (1.26 g, 20 mmol) was then added and the resulting solution was stirred at 20° C. for 12 h. Boc₂O (3.57 g, 16.4 mmol) and TEA (3 mL) were then added and the solution was stirred at 20° C. for another 2 h until LCMS showed the reaction complete. The solution was then diluted with water and taken up with DCM (300 mL), washed with aq. NH₄Cl and water then the organic phase was dried, solvent removed and the residue purified with column chromatographic separation to afford the desired product as white solid (4.19 g, yield: 88%). LCMS: 292.2 (M+1).

### Step 2: tert-butyl cyclopentyl(3-(oxiran-2-ylmethoxy)benzyl)carbamate

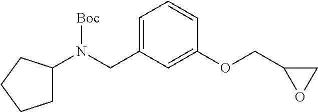 [SMILES: CC(C)(C)OC(=O)N(Cc1cccc(OCC2CO2)c1)C1CCCC1]

To a stirring solution of tert-butyl cyclopentyl(3-hydroxybenzyl)carbamate (500 mg, 1.72 mmol) in acetonitrile was added K₂CO₃ (276 mg, 2 mmol) followed by 2-(bromomethyl)oxirane (247 mg, 1.8 mmol) and the resulting mixture was stirred at 60° C. for 3 h. Upon completion of the reaction the reaction solvent was removed and the residue purified with column separation to afford the desired product as a colorless oil (447 mg, yield 75%). LCMS: 348.2 (M+1).

### Step 3: 1-(3-((cyclopentylamino)methyl)phenoxy)-3-(3,4-dihydro-2,7-naphthyridin-2(1H)-yl)propan-2-ol

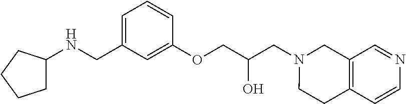 [SMILES: OC(COc1cccc(CNC2CCCC2)c1)CN1CCc2ccncc2C1]

To a solution of tert-butyl cyclopentyl(3-(oxiran-2-ylmethoxy)benzyl)carbamate (200 mg, 0.58 mmol) in EtOH (5 mL) was added 1,2,3,4-tetrahydro-2,7-naphthyridine (100 mg, 0.75 mmol) and the resulting solution was then heated at 80° C. for 5 h. The mixtures was then cooled to 20° C., and HCl in MeOH (4N, 2 mL) was then added before stirring the reaction mixture for a further another 3 h at 20° C. Once the reaction was complete by LCMS analysis the solvents were evaporated to dryness and the residue purified by Pre-HPLC to afford the desired product (95 mg, yield: 43.0%). ¹H NMR (CD₃OD, 400 MHz) δ (ppm): 8.82 (s, 1H), 8.73 (d, J=6.0 Hz, 1H), 7.94 (d, J=6.0 Hz, 1H), 7.40 (dd, J₁=J₂=8 Hz, 1H), 7.16-7.04 (m, 3H), 4.84 (s, 2H), 4.60-4.57 (m, 1H), 4.17 (s, 2H), 4.12-4.10 (m, 2H), 3.87-3.80 (m, 2H), 3.65-3.52 (m, 5H), 2.19-2.12 (m, 2H), 1.84-1.73 (m, 6H); LCMS:382.1 (M+1).

### Example 20: (S)—N-(3-(6,7-dihydro-1H-imidazo[4,5-c]pyridin-5(4H)-yl)-2-hydroxypropyl)-6-(oxetan-3-ylamino)pyrimidine-4-carboxamide (Compound 3-38)

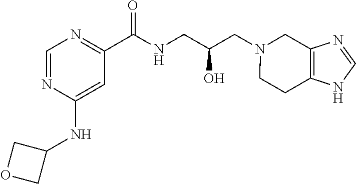 [SMILES: O=C(NC[C@H](O)CN1CCc2[nH]cnc2C1)c1cc(NC2COC2)ncn1]

### Step 1: 1-((2-(trimethylsilyl)ethoxy)methyl)-4,5,6,7-tetrahydro-1H-imidazo[4,5-c]pyridine

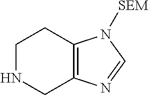 [SMILES: C[Si](C)(C)CCOCn1cnc2c1CCNC2]

To a solution of tert-butyl 1-((2-(trimethylsilyl)ethoxy)methyl)-6,7-dihydro-1H-imidazo[4,5-c]pyridine-5(4H)-carboxylate (5.0 g, 14.2 mmol) in DCM (50 mL) was added TFA (8 mL). The mixture was stirred at 25° C. for 16 h. The mixture was then evaporated to give the target compound 1-((2-(trimethylsilyl)ethoxy)methyl)-4,5,6,7-tetrahydro-1H-imidazo[4,5-c]pyridine (3.59 g, yield: 100%).

### Step 2: (R)-5-(oxiran-2-ylmethyl)-1-((2-(trimethylsilyl)ethoxy)methyl)-4,5,6,7-tetrahydro-1H-imidazo[4,5-c]pyridine

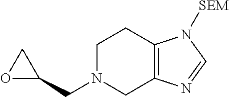 [SMILES: C[Si](C)(C)CCOCn1cnc2c1CCN(C[C@@H]1CO1)C2]

To a solution of 1-((2-(trimethylsilyl)ethoxy)methyl)-4,5,6,7-tetrahydro-1H-imidazo[4,5-c]pyridine (3.59 g, 14.2 mmol) in THF (50 mL) was added (S)-oxiran-2-ylmethyl 3-nitrobenzenesulfonate (3.68 g, 14.2 mmol) and KF (3.29 g, 56.8 mmol). The mixture was stirred at 25° C. for 16 hours. The reaction mixture was filtered and the mixture was used directly for the next step.

### Step 3: (S)-1-amino-3-(3-((2-(trimethylsilyl)ethoxy)methyl)-6,7-dihydro-3H-imidazo[4,5-c]pyridin-5(4H)-yl)propan-2-ol

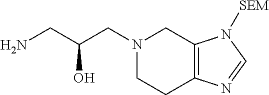 [SMILES: C[Si](C)(C)CCOCn1cnc2c1CN(C[C@@H](O)CN)CC2]

To the solution pf (R)-5-(oxiran-2-ylmethyl)-1-((2-(trimethylsilyl)ethoxy)methyl)-4,5,6,7-tetrahydro-1H-imidazo[4,5-c]pyridine in THF (50 mL) and EtOH (50 mL) was added NH₃.H₂O (30 mL). The mixture was stirred at 25° C. for 16 h. TLC showed the reaction completed and the solvent was evaporated, residue was purified by column separation to afford desired product (2.0 g, 43% two steps). LCMS (m/z): 327.2 [M+H]⁺

### Step 4: (S)-6-chloro-N-(2-hydroxy-3-(3-((2-(trimethylsilyl)ethoxy)methyl)-6,7-dihydro-3H-imidazo[4,5-c]pyridin-5(4H)-yl)propyl)pyrimidine-4-carboxamide

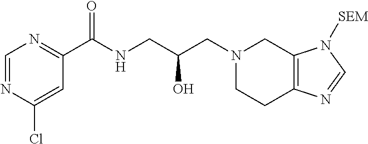 [SMILES: C[Si](C)(C)CCOCn1cnc2c1CN(C[C@@H](O)CNC(=O)c1cc(Cl)ncn1)CC2]

To a solution of (S)-1-amino-3-(3-((2-(trimethylsilyl)ethoxy)methyl)-6,7-dihydro-3H-imidazo[4,5-c]pyridin-5(4H)-yl)propan-2-ol (3.26 g, 10 mmol) in DCM (50 mL) was added TEA (2.02 g, 20 mmol). The solution was cooled to 0° C. and 6-chloropyrimidine-4-carbonyl chloride (1.77 g, 10 mmol) was then added. The solution was stirred at 25° C. for 1 h, then the solution was then taken up with DCM, washed with H₂O (50 mL) and the DCM layer was combined and concentrated. The residue was purified by silica column (DCM/MeOH=10:1) to give the (S)-6-chloro-N-(2-hydroxy-3-(3-((2-(trimethylsilyl)ethoxy)methyl)-6,7-dihydro-3H-imidazo[4,5-c]pyridin-5(4H)-yl)propyl)pyrimidine-4-carboxamide. (850 mg, yield: 18.2%). LCMS (m/z): 467.1 [M+H]⁺

### Step 5: (S)—N-(3-(6,7-dihydro-1H-imidazo[4,5-c]pyridin-5(4H)-yl)-2-hydroxypropyl)-6-(oxetan-3-ylamino)pyrimidine-4-carboxamide

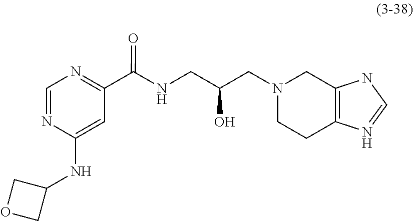 [SMILES: O=C(NC[C@H](O)CN1CCc2nc[nH]c2C1)c1cc(NC2COC2)ncn1]

To a solution of (S)-6-chloro-N-(2-hydroxy-3-(3-((2-(trimethylsilyl)ethoxy)methyl)-6,7-dihydro-3H-imidazo[4,5-c]pyridin-5(4H)-yl)propyl)pyrimidine-4-carboxamide (300 mg, 0.64 mmol) in DCM (10 mL) was added TFA (2 mL). The mixture was stirred at 25° C. for 2 h until the reaction was shown to be complete by TLC analysis. The solvent was evaporated to give the crude (S)-6-chloro-N-(3-(6,7-dihydro-1H-imidazo[4,5-c]pyridin-5(4H)-yl)-2-hydroxypropyl)pyrimidine-4-carboxamide. This crude was re-dissolved in i-PrOH (5 mL), oxetan-3-amine (46.1 mg, 0.64 mmol) and TEA (129.3 mg, 1.28 mmol) were added and the mixture was heated at 80° C. for 16 hours. After that, the reaction mixture was concentrated to dryness and the residue was purified by prep-HPLC (basic condition with ammonium) to give the target compound (S)—N-(3-(6,7-dihydro-1H-imidazo[4,5-c]pyridin-5(4H)-yl)-2-hydroxypropyl)-6-(oxetan-3-ylamino)pyrimidine-4-carboxamide. (22.3 mg, yield: 9.3%). ¹H NMR (400 MHz, CD₃OD-d₄): δ (ppm): =8.46 (s, 1H), 8.12 (s, 1H), 7.16 (br. s., 1H), 5.12 (br. s., 1H), 4.96 (t, J=6.8 Hz, 2H), 4.61 (t, J=6.3 Hz, 2H), 4.35-4.21 (m, 2H), 3.61-3.46 (m, 4H), 3.28-3.12 (m, 2H), 3.08-2.93 (m, 2H). LCMS (m/z): 374.2 [M+H]⁺

### Example 21: (S)-6-((1-acetylazetidin-3-yl)amino)-N-(3-(6,7-dihydro-1H-imidazo[4,5-c]pyridin-5(4H)-yl)-2-hydroxypropyl)pyrimidine-4-carboxamide (Compound 3-33)

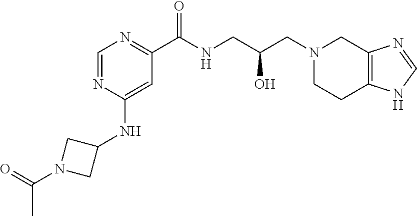 [SMILES: CC(=O)N1CC(Nc2cc(C(=O)NC[C@H](O)CN3CCc4[nH]cnc4C3)ncn2)C1]

To a solution of (S)-6-chloro-N-(2-hydroxy-3-(3-((2-(trimethylsilyl)ethoxy)methyl)-6,7-dihydro-3H-imidazo[4,5-c]pyridin-5(4H)-yl)propyl)pyrimidine-4-carboxamide (250 mg, 0.54 mmol) in i-PrOH (5 mL) was added 1-(3-aminoazetidin-1-yl)ethanone (61.6 mg, 0.54 mmol) and TEA (109.1 mg, 1.08 mmol). The mixture was stirred at 80° C. for 16 hours. The reaction mixture was then evaporated to dryness and the residue was re-dissolved in DCM (10 mL), TFA (2 mL) was added. The mixture was stirred at 25° C. for another 16 h. TLC showed the reaction worked well. The solvent was evaporated and the residue was purified by prep-HPLC to give the target. (14.1 mg, yield: 6.3%). ¹H NMR (400 MHz, CD₃OD-d₄): δ (ppm): =8.65 (s, 1H), 8.53 (s, 1H), 7.18 (br. s., 1H), 4.78 (br. s., 1H), 4.58 (t, J=8.4 Hz, 1H), 4.51 (s, 2H), 4.38-4.28 (m, 2H), 4.10 (dd, J=5.1, 8.9 Hz, 1H), 3.89 (dd, J=5.0, 10.0 Hz, 1H), 3.71 (br. s., 2H), 3.60-3.37 (m, 4H), 3.11 (d, J=5.8 Hz, 2H), 1.90 (s, 3H). LCMS (m/z): 415.3 [M+H]⁺

### Example 22: (S)-6-((1-acetylpiperidin-4-yl)amino)-N-(3-(6,7-dihydro-1H-imidazo[4,5-c]pyridin-5(4H)-yl)-2-hydroxypropyl)pyrimidine-4-carboxamide (Compound 3-34)

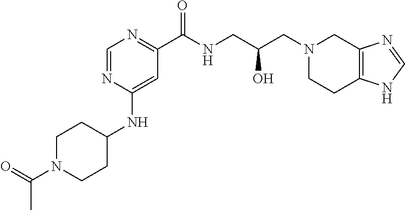 [SMILES: CC(=O)N1CCC(Nc2cc(C(=O)NC[C@H](O)CN3CCc4[nH]cnc4C3)ncn2)CC1]

To a solution of (S)-6-chloro-N-(2-hydroxy-3-(3-((2-(trimethylsilyl)ethoxy)methyl)-6,7-dihydro-3H-imidazo[4,5-c]pyridin-5(4H)-yl)propyl)pyrimidine-4-carboxamide (250 mg, 0.54 mmol) in i-PrOH (5 mL) was added 1-(4-aminopiperidin-1-yl)ethanone (76.9 mg, 0.54 mmol) and TEA (109.1 mg, 1.08 mmol). The mixture was stirred at 80° C. for 16 hours. The reaction mixture was evaporated and the residue was dissolved in DCM (10 mL), TFA (2 mL) was then added. The mixture was stirred at 25° C. for 16 h. The mixture was then evaporated to dryness and the residue was purified by prep-HPLC to give the desired product (23.7 mg, yield: 10%). ¹H NMR (400 MHz, CD₃OD-d₄): δ (ppm): =8.72 (s, 1H), 8.51 (s, 1H), 7.15 (br. s., 1H), 4.55 (s, 2H), 4.45 (d, J=13.1 Hz, 1H), 4.36-4.15 (m, 2H), 3.95 (d, J=13.6 Hz, 1H), 3.74 (br. s., 2H), 3.62-3.34 (m, 4H), 3.27-3.02 (m, 3H), 2.89 (t, J=11.5 Hz, 1H), 2.18-1.98 (m, 5H), 1.60-1.39 (m, 2H). LCMS (m/z): 443.3 [M+H]⁺

### Example 23: (S)-6-((1-acetylpiperidin-4-yl)amino)-N-(2-hydroxy-3-(3-methyl-4,5-dihydrothieno[2,3-c]pyridin-6(7H)-yl)propyl)pyrimidine-4-carboxamide (Compound 1-10)

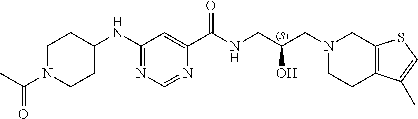

### Step 1: 2,3-dibromo-4,5,6,7-tetrahydrothieno[2,3-c]pyridine

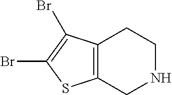 [SMILES: Brc1sc2c(c1Br)CCNC2]

To a solution of 4,5,6,7-tetrahydrothieno[2,3-c]pyridine (220 mg, 1.58 mmol) in AcOH (5 mL) was added Br₂ (1.26 g, 7.91 mmol). The mixture was stirred at 80° C. for 16 h. The solid was precipitated and collected by filtration to give the 2,3-dibromo-4,5,6,7-tetrahydrothieno[2,3-c]pyridine (200 mg, yield: 42.6%). LCMS (m/z): 297.9 [M+H]⁺

### Step 2: 3-bromo-4,5,6,7-tetrahydrothieno[2,3-c]pyridine

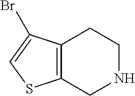

To a solution of 2,3-dibromo-4,5,6,7-tetrahydrothieno[2,3-c]pyridine (200 mg, 0.673 mmol) in AcOH (5 mL) was added Zn dust (88.1 mg, 1.35 mmol) and HCl(0.1 mL). The mixture was stirred at 80° C. for 16 h. The solvent was then evaporated and the residue was purified by preparative TLC (DCM/MeOH=10:1) to give 3-bromo-4,5,6,7-tetrahydrothieno[2,3-c]pyridine (120 mg, yield: 81.8%). LCMS (m/z): 220.0 [M+H]⁺

### Step 3: 3-methyl-4,5,6,7-tetrahydrothieno[2,3-c]pyridine

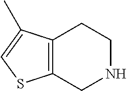

To a solution of 3-bromo-4,5,6,7-tetrahydrothieno[2,3-c]pyridine (500 mg, 2.29 mmol) in dioxane (10 mL) and H₂O (2 mL) was added methylboronic acid (206.1 mg, 3.44 mmol), Pd(dppf)Cl₂ (168.1 mg, 0.23 mmol) and K₂CO₃ (632 mg, 4.58 mmol). The resulting mixture was stirred at 100° C. for 16 hours. The reaction mixture was filtered and the filtrate was concentrated and the residue was purified by preparative TLC (DCM/MeOH=10:1) to give 3-methyl-4,5,6,7-tetrahydrothieno[2,3-c]pyridine (190 mg, yield: 57.1%). LCMS (m/z): 154.0 [M+H]⁺

### Step 4: (R)-3-methyl-6-(oxiran-2-ylmethyl)-4,5,6,7-tetrahydrothieno[2,3-c]pyridine

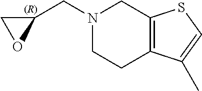

To a mixture of 3-methyl-4,5,6,7-tetrahydrothieno[2,3-c]pyridine (190 mg, 1.24 mmol), KF (288 mg, 4.96 mmol) and K₂CO₃ (171 mg, 1.24 mmol) in THF (40 mL) was added (S)-oxiran-2-ylmethyl 3-nitrobenzenesulfonate (386 mg, 1.49 mmol) at 16° C. The mixture was stirred at 40° C. for 30 h, at which time LCMS showed the completion of the reaction. The mixture was filtered and concentrated to give the crude product (510 mg, crude), which was used in next step without further purification. LCMS (m/z): 210.1 [M+H]⁺.

### Step 5: (S)-1-amino-3-(3-methyl-4,5-dihydrothieno[2,3-c]pyridin-6(7H)-yl)propan-2-ol

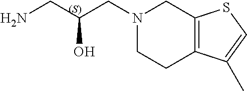

To the stirring solution of (R)-1-methyl-5-(oxiran-2-ylmethyl)-4,5,6,7-tetrahydro-1H-pyrazolo [4,3-c]pyridine (510 mg, crude) in EtOH (50 mL) was added NH₃/H₂O (100 mL) at 18° C. The mixture was stirred at 18° C. for 12 h. TLC showed the reaction completed, then solution was concentrated to give the cured product (370 g, crude), which was used directly in next step without further purification. LCMS (m/z): 227.1 [M+H]⁺.

### Step 6: (S)-6-chloro-N-(2-hydroxy-3-(3-methyl-4,5-dihydrothieno[2,3-c]pyridin-6(7H)-yl)propyl)pyrimidine-4-carboxamide

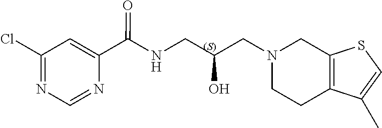

To a stirring solution of (S)-1-amino-3-(1-methyl-6,7-dihydro-1H-pyrazolo[4,3-c]pyridin-5(4H)-yl)propan-2-ol (370 mg, crude) in DCM (20 mL) and Et₃N (1 mL) was added 6-chloropyrimidine-4-carbonyl chloride (219 mg, 1.24 mmol) at 17° C. The resulting solution was stirred at this temperature for 3 h, LCMS showed the reaction completed, the reaction mixture was diluted with water (50 mL), extracted with DCM (30 mL×3), the organic layer was combined and solvent was evaporated. The residue was then purified by preparative TLC to give the compound as brown oil (160 mg, 35.3%). LCMS (m/z): 367.1 [M+H]⁺.

### Step 7: (S)-6-((1-acetylpiperidin-4-yl)amino)-N-(2-hydroxy-3-(3-methyl-4,5-dihydrothieno[2,3-c]pyridin-6(7H)-yl)propyl)pyrimidine-4-carboxamide

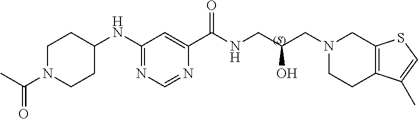

To a mixture of (S)-6-chloro-N-(2-hydroxy-3-(1-methyl-6,7-dihydro-1H-pyrazolo[4,3-c]pyridine-5(4H)-yl)propyl)pyrimidine-4-carboxamidel (80 mg, 0.22 mmol) and 1-(4-aminopiperidin-1-yl) ethanone (62 mg, 0.44 mmol) in i-PrOH (10 mL) was added Et₃N (0.1 mL) at 19° C. The mixture was stirred at 90° C. for 12 h or until the reaction was shown to be complete by LCMS analysis. The reaction mixture was then concentrated to give the crude product which was purified by prep-HPLC to give the target compound (S)-6-((1-acetylpiperidin-4-yl)amino)-N-(2-hydroxy-3-(3-methyl-4,5-dihydrothieno[2,3-c]pyridin-6(7H)-yl)propyl)pyrimidine-4-carboxamide as yellow oil (15.5 mg, 14.9%). ¹H NMR (MeOD, 400 MHz) δ (ppm): 8.37 (s, 1H) 7.11 (s, 1H) 6.83 (s, 1H) 4.46 (d, J=13.55 Hz, 1H) 4.19 (br. s., 1H) 4.09 (dt, J=11.98, 5.93 Hz, 1H) 3.95 (d, J=12.80 Hz, 1H) 3.86 (s, 2H) 3.53-3.58 (m, 1H) 3.45-3.51 (m, 1H) 3.28 (br. s., 1H) 2.96-3.06 (m, 2H) 2.91 (t, J=11.29 Hz, 1H) 2.73-2.82 (m, 2H) 2.64-2.72 (m, 2H) 2.13 (d, J=6.78 Hz, 6H) 1.94-2.10 (m, 1H) 1.40-1.54 (m, 2H). LCMS (m/z): 473.2 [M+H]⁺.

### Example 24: (S)—N-(2-hydroxy-3-(3-methyl-6,7-dihydrothieno[3,2-c]pyridin-5(4H)-yl)propyl)-6-(oxetan-3-ylamino)pyrimidine-4-carboxamide (Compound 1-11)

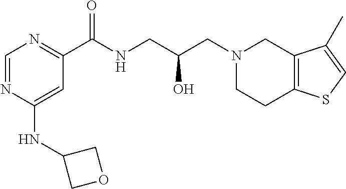 [SMILES: Cc1csc2c1CN(C[C@@H](O)CNC(=O)c1cc(NC3COC3)ncn1)CC2]

### Step 1: tert-butyl 6,7-dihydrothieno[3,2-c]pyridine-5(4H)-carboxylate

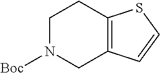 [SMILES: CC(C)(C)OC(=O)N1CCc2sccc2C1]

To a stirring solution of 4,5,6,7-tetrahydrothieno[3,2-c]pyridine (5.6 g, 40.3 mmol), and Et₃N (6.1 g, 60.4 mmol) in MeOH (100 mL) was added Boc₂O (10.5 g, 48.4 mmol) in 30 min at 0° C. The mixture was stirred at 20° C. for 16 hours. The reaction mixture was concentrated after the starting material was consumed and the residue re-dissolved in DCM, the organic solution washed with water and aq. HCl (0.5 M), the organic phases was dried and concentrated to give 8.9 g (yield: 92.4%) of tert-butyl 6,7-dihydrothieno[3,2-c]pyridine-5(4H)-carboxylate as white solid. 1H NMR (400 MHz, CDCl₃) δ (ppm): 7.14 (d, J=5.0 Hz, 1H), 6.81 (d, J=5.0 Hz, 1H), 4.52 (br. s, 2H), 3.75 (br. s, 2H), 2.87 (br. s, 2H), 1.51 (s, 9H). LCMS (m/z): 240.2 [M+H]⁺

### Step 2: 2,3-dibromo-4,5,6,7-tetrahydrothieno[3,2-c]pyridine

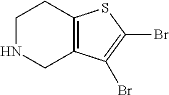 [SMILES: Brc1sc2c(c1Br)CNCC2]

To a stirring solution of tert-butyl 6,7-dihydrothieno[3,2-c]pyridine-5(4H)-carboxylate (8.9 g, 37.2 mmol) in CHCl₃ (100 mL) was added Br₂ (23.8 g, 149 mmol) at 0° C. Then the mixture was stirred at 60° C. for 16 hours. Reaction was then quenched by aq. Na₂SO₃, and extracted with DCM, organic layer was washed with aq.Na₂CO₃ and water, dried and concentrated to give (8.3 g, yield: 75.5%) of crude 2,3-dibromo-4,5,6,7-tetrahydrothieno[3,2-c]pyridine which was used in next step without purification. LCMS (m/z): 297.9 [M+H]⁺

### Step 3: tert-butyl 2,3-dibromo-6,7-dihydrothieno[3,2-c]pyridine-5(4H)-carboxylate

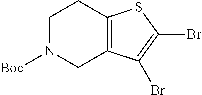 [SMILES: CC(C)(C)OC(=O)N1CCc2sc(Br)c(Br)c2C1]

To the stirring solution of crude 2, 3-dibromo-4,5,6,7-tetrahydrothieno[3,2-c]pyridine (8.3 g, 28.2 mmol) in DCM (100 mL) and Et₃N (6.1 g, 60.4 mmol) was added Boc₂O (7.4 g, 33.8 mmol) in 30 min at 0° C. and the resulting solution was then stirred at 20° C. for another 3 h, and taken up with DCM, washed with aq. HCl. The organic layer was dried and concentrated to give 6.7 g (yield: 60.2%) of crude tert-butyl 2,3-dibromo-6,7-dihydrothieno[3,2-c]pyridine-5(4H)-carboxylate as white solid. LCMS (m/z): 397.9 [M+H]⁺

### Step 4: 3-bromo-4,5,6,7-tetrahydrothieno[3,2-c]pyridine

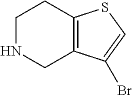

To a stirring mixture of tert-butyl 2,3-dibromo-6,7-dihydrothieno[3,2-c]pyridine-5(4H)-carboxylate (6.7 g, 16.9 mmol) in AcOH/H₂O (50 mL/50 mL) was added Zn dust (4.7 g, 84.8 mmol). Then the mixture was stirred at 60° C. for 16 hours. The reaction mixture was concentrated to give crude 3-bromo-4,5,6,7-tetrahydrothieno[3,2-c]pyridine which was used in next step without purified. LCMS (m/z): 218.2/220.2 [M+H]⁺

### Step 5: tert-butyl 3-bromo-6,7-dihydrothieno[3,2-c]pyridine-5(4H)-carboxylate

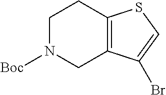 [SMILES: CC(C)(C)OC(=O)N1CCc2scc(Br)c2C1]

The mixture of crude 3-bromo-4,5,6,7-tetrahydrothieno[3,2-c]pyridine and Boc₂O (4.3 g, 20 mmol), Et3N (2.1 g, 21 mmol) in MeOH (100 mL) was stirred at 20° C. for 2 hours. The reaction mixture was concentrated and the residue was dissolved in DCM, the mixture was washed by water and aq. HCl (0.5 M), the organic phases was concentrated and the residue was purified by column to give 4.7 g (yield: 86.2%) of tert-butyl 3-bromo-6,7-dihydrothieno[3,2-c]pyridine-5(4H)-carboxylate as white solid. ¹H NMR (400 MHz, CDCl₃) δ (ppm): 7.11 (s, 1H), 4.39 (br. s, 2H), 3.80-3.65 (m, 2H), 2.84 (br. s., 2H), 1.54-1.50 (m, 9H). LCMS (m/z): 318.2/320.2 [M+H]⁺

### Step 6: tert-butyl 3-methyl-6,7-dihydrothieno[3,2-c]pyridine-5(4H)-carboxylate

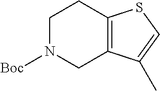 [SMILES: Cc1csc2c1CN(C(=O)OC(C)(C)C)CC2]

The mixture of crude tert-butyl 3-bromo-6,7-dihydrothieno[3,2-c]pyridine-5(4H)-carboxylate (4.7 g, 14.8 mmol) and 2,4,4,5,5-pentamethyl-1,3,2-dioxaborolane (3.15 g, 22.2 mmol), H₂O (360 mg, 22 mmol), Pd(dppf)₂Cl₂ (0.5 g, 10% w) in dioxane (100 mL) was stirred at 100° C. for 2 hours. The reaction mixture was concentrated and the residue was purified by column chromatography to give 2.0 g (yield: 86.2%) of tert-butyl 3-methyl-6,7-dihydrothieno[3,2-c]pyridine-5(4H)-carboxylate as white solid. 1H NMR (400 MHz, CDCl₃) δ (ppm): 6.75 (s, 1H), 4.39 (br. s, 2H), 3.72 (br. s, 2H), 2.83 (br. s, 2H), 2.13 (s, 3H), 1.517 (m, 9H). LCMS (m/z): 254.2 [M+H]⁺

### Step 7: 3-methyl-4,5,6,7-tetrahydrothieno[3,2-c]pyridine

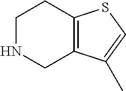

To a stirring solution of tert-butyl 3-methyl-6,7-dihydrothieno[3,2-c]pyridine-5(4H)-carboxylate (2.2 g, 8.7 mmol) in EA (30 mL) was added 4N HCl/ethyl acetate (5 mL) at 0° C., then the mixture was stirred at 25° C. for 12 h. The reaction was concentrated to give the crude product (1.5 g, 115% yield). LCMS (m/z): 154.2 [M+H]⁺

### Step 8: (R)-3-methyl-5-(oxiran-2-ylmethyl)-4,5,6,7-tetrahydrothieno[3,2-c]pyridine

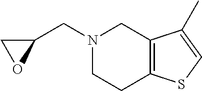

A mixture of 3-methyl-4,5,6,7-tetrahydrothieno[3,2-c]pyridine (1.3 g, 8.7 mmol), (S)-oxiran-2-ylmethyl 3-nitrobenzenesulfonate(2.7 g, 10.4 mmol), TEA (1.8 g, 17.6 mmol) and KF (5.5 g, 34.8 mmol) in THF (50 mL) was stirred at 25° C. for 16 h. The solid was filter and the filtrate was concentrated to give the crude product (2.0 g, 111% yield), which was used in the next step without further purification.

### Step 9: (S)-1-amino-3-(3-methyl-6,7-dihydrothieno[3,2-c]pyridin-5(4H)-yl)propan-2-ol

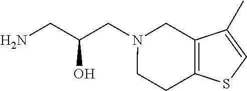

To a solution of (R)-3-methyl-5-(oxiran-2-ylmethyl)-4,5,6,7-tetrahydrothieno[3,2-c]pyridine (1.8 g, 8.7 mmol) in EtOH (100 mL) was added NH₃.H₂O (50 mL) and stirred at 25° C. for 16 h. After the starting material was consumed, the reaction was cooled to room temperature and the solvent removed under vacuum to give the crude product which was then purified by chromatography on silica gel (DCM:MeOH 10:1) to give the desired product (1.5 g, 79% yield). LCMS (m/z): 227.1 [M+H]⁺

### Step 10: (S)-6-chloro-N-(2-hydroxy-3-(3-methyl-6,7-dihydrothieno[3,2-c]pyridin-5(4H)-yl)propyl)pyrimidine-4-carboxamide

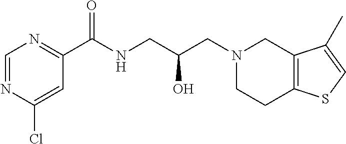 [SMILES: Cc1csc2c1CN(C[C@@H](O)CNC(=O)c1cc(Cl)ncn1)CC2]

To the stirring solution of (S)-1-amino-3-(3-methyl-6,7-dihydrothieno[3,2-c]pyridin-5(4H)-yl)propan-2-ol (1 g, 4.4 mmol) in DCM (50 mL) and TEA (1 mL) was added 6-chloropyrimidine-4-carbonyl chloride (774 mg, 4.4 mmol, in 10 mL DCM) at 0° C., the mixture was stirred at 25° C. for another 4 h. After that, the reaction solution was poured into 50 mL of ice-water, extracted with DCM (50 mL×3) and dried over Na₂SO₄. The solvent was removed by concentration to give the crude product and then purified by preperative-TLC separation to give the desired product (450 mg, 28% yield); LCMS (m/z): 367.0 (M+1).

### Step 11: (S)—N-(2-hydroxy-3-(3-methyl-6,7-dihydrothieno[3,2-c]pyridin-5(4H)-yl)propyl)-6-(oxetan-3-ylamino)pyrimidine-4-carboxamide

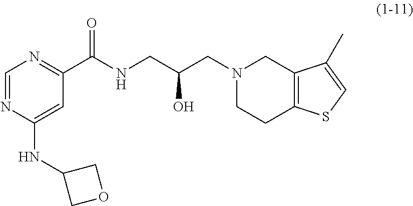 [SMILES: Cc1csc2c1CN(C[C@@H](O)CNC(=O)c1cc(NC3COC3)ncn1)CC2]

A mixture of (S)-6-chloro-N-(2-hydroxy-3-(3-methyl-6,7-dihydrothieno[3,2-c]pyridin-5(4H)-yl)propyl)pyrimidine-4-carboxamide (150 mg, 0.4 mmol), oxetan-3-amine (58 mg, 0.8 mmol) and TEA (0.1 mL) in i-PrOH (10 mL) was stirred at 80° C. for 12 h. Then the solvent was removed to give the crude product and purified by HPLC separation to give the desired product (33.9 mg, 21% yield). ¹H NMR (400 MHz, METHANOL-d4) δ (ppm): 8.35 (s, 1H), 7.15 (br. s., 1H), 6.76 (s, 1H), 5.12 (br. s., 1H), 4.98 (t, J=6.9 Hz, 2H), 4.62 (t, J=6.3 Hz, 2H), 4.08 (quin, J=5.9 Hz, 1H), 3.60-3.46 (m, 4H), 2.95-2.83 (m, 4H), 2.72 (d, J=6.0 Hz, 2H), 2.07 (s, 3H); LCMS (m/z): 404.1 (M+1).

### Example 25: (S)-6-((1-acetylazetidin-3-yl)amino)-N-(2-hydroxy-3-(3-methyl-6,7-dihydrothieno[3,2-c]pyridin-5(4H)-yl)propyl)pyrimidine-4-carboxamide (Compound 1-12)

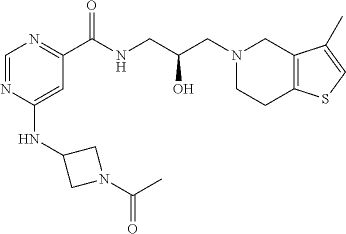 [SMILES: CC(=O)N1CC(Nc2cc(C(=O)NC[C@H](O)CN3CCc4scc(C)c4C3)ncn2)C1]

A mixture of (S)-6-chloro-N-(2-hydroxy-3-(3-methyl-6,7-dihydrothieno[3,2-c]pyridin-5(4H)-yl)propyl)pyrimidine-4-carboxamide (150 mg, 0.4 mmol), 1-(3-aminoazetidin-1-yl)ethanone (91 mg, 0.8 mmol) and TEA (0.2 mL) in i-PrOH (10 mL) was stirred at 80° C. for 12 h. Then the solvent was removed to give the crude product which purified by Pre-HPLC separation to give the desired product (48.6 mg, 27% yield). 1H NMR (400 MHz, METHANOL-d4) δ (ppm): 8.39 (s, 1H), 7.16 (br. s., 1H), 6.76 (s, 1H), 5.12 (br. s., 1H), 4.98 (t, J=6.9 Hz, 2H), 4.62 (t, J=6.3 Hz, 2H), 4.08 (quin, J=5.9 Hz, 1H), 3.60-3.46 (m, 4H), 2.95-2.83 (m, 4H), 2.72 (d, J=6.0 Hz, 2H), 2.07 (s, 3H). LCMS (m/z): 445.2 (M+1)

### Example 26: (S)-6-((1-acetylpiperidin-4-yl)amino)-N-(2-hydroxy-3-(3-methyl-6,7-dihydrothieno[3,2-c]pyridin-5(4H)-yl)propyl)pyrimidine-4-carboxamide (Compound 1-13)

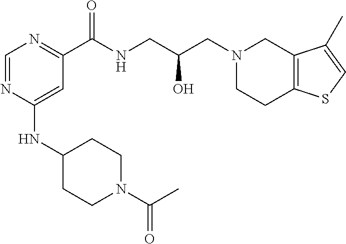 [SMILES: CC(=O)N1CCC(Nc2cc(C(=O)NC[C@H](O)CN3CCc4scc(C)c4C3)ncn2)CC1]

A mixture of (S)-6-chloro-N-(2-hydroxy-3-(3-methyl-6,7-dihydrothieno[3,2-c]pyridin-5(4H)-yl)propyl)pyrimidine-4-carboxamide (150 mg, 0.4 mmol), 1-(4-aminopiperidin-1-yl)ethanone (113 mg, 0.8 mmol) and TEA (0.1 mL) in i-PrOH (10 mL) was stirred at 80° C. for 12 h. Then the solvent was removed to give the crude product and purified by Pre-HPLC separation to give the desired product (54.1 mg, 28% yield). ¹H NMR (400 MHz, METHANOL-d4) δ (ppm): 8.34 (s, 1H), 7.10 (s, 1H), 6.76 (s, 1H), 4.45 (d, J=13.6 Hz, 1H), 4.18 (br. s., 1H), 4.11-4.04 (m, 1H), 3.95 (d, J=13.8 Hz, 1H), 3.62-3.52 (m, 3H), 3.52-3.37 (m, 1H), 3.32-3.23 (m, 1H), 2.94 (d, J=3.8 Hz, 1H), 2.91-2.78 (m, 4H), 2.72 (d, J=6.0 Hz, 2H), 2.19-2.12 (m, 3H), 2.12-1.89 (m, 5H), 1.60-1.38 (m, 2H); LCMS (m/z): 459.1 (M+1).

### Example 27: (S)-2-((1-acetylpiperidin-4-yl)amino)-N-(2-hydroxy-3-(3-methyl-6,7-dihydrothieno[3,2-c]pyridin-5(4H)-yl)propyl)isonicotinamide (Compound 1-14)

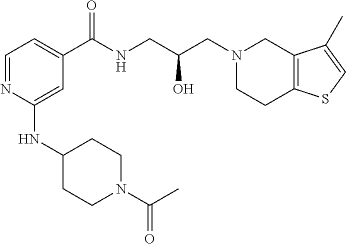 [SMILES: CC(=O)N1CCC(Nc2cc(C(=O)NC[C@H](O)CN3CCc4scc(C)c4C3)ccn2)CC1]

A mixture of 2-((1-acetylpiperidin-4-yl)amino)isonicotinic acid (174 mg, 0.66 mmol), (S)-1-amino-3-(3-methyl-6,7-dihydrothieno[3,2-c]pyridin-5(4H)-yl)propan-2-ol (150 mg, 0.66 mmol), HATU (259 mg, 0.66 mmol) and TEA (1 mL) in DCM (10 mL) was stirred at 20° C. for 12 h. Then the solvent was removed to give the crude product and purified by Pre-HPLC separation to give the desired product (29 mg, 9.4% yield). 1H NMR (400 MHz, METHANOL-d₄) δ (ppm): 7.97 (d, J=5.5 Hz, 1H), 6.88 (s, 1H), 6.81-6.73 (m, 2H), 4.44 (d, J=13.6 Hz, 1H), 4.10 (td, J=6.0, 11.9 Hz, 1H), 4.01-3.90 (m, 2H), 3.56 (s, 2H), 3.53-3.43 (m, 2H), 3.31-3.25 (m, 1H), 2.97-2.84 (m, 5H), 2.79-2.68 (m, 2H), 2.14 (s, 3H), 2.08 (s, 3H), 2.04 (d, J=14.8 Hz, 1H), 1.55-1.45 (m, 1H), 1.44-1.35 (m, 1H); LCMS (m/z): 472.3 (M+1).

### Example 28: (S)-2-((1-acetylpiperidin-4-yl)amino)-N-(3-(2,3-dimethyl-6,7-dihydrothieno[3,2-c]pyridin-5(4H)-yl)-2-hydroxypropyl)isonicotinamide (Compound 1-15)

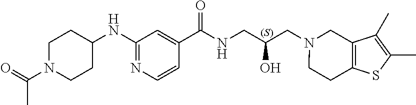

### Step 1: tert-butyl 2-methyl-6,7-dihydrothieno[3,2-c]pyridine-5(4H)-carboxylate

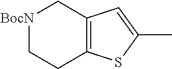 [SMILES: Cc1cc2c(s1)CCN(C(=O)OC(C)(C)C)C2]

Tert-butyl 6,7-dihydrothieno[3,2-c]pyridine-5(4H)-carboxylate (10.0 g, 41.8 mmol) was dissolved in THF (150 mL) and the solution was cooled to −78° C. under dry ice-acetone bath, n-BuLi (25 mL, 62.8 mmol) was added dropwise under N₂. After that the reaction solution was stirred at −78° C. for 30 min and MeI (8.9 g, 62.8 mmol) was then added at this temperature. The resulting mixture was allowed to warm to 20° C. and stirred at this temperature for 4 h under N₂. The reaction was quenched with water (20 mL), and the mixture was diluted with EA and washed with water. The organic phase was dried and concentrated, and the residue was purified by column chromatography (petroleum ether:ethyl acetate=20:1) to give the desired product (9.0 g, 84.9%). LCMS (m/z): 198.1 [M+H−56]⁺

### Step 2: 3-bromo-2-methyl-4,5,6,7-tetrahydrothieno[3,2-c]pyridine

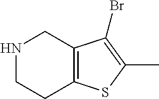

To a solution of tert-butyl 2-methyl-6,7-dihydrothieno[3,2-c]pyridine-5(4H)-carboxylate (10.0 g, 39.5 mmol) in CHCl₃ (100 mL) was added dropwise Br₂ (12.6 g, 79.0 mmol) at 20° C. The stirring solution was then heated at 70° C. for 16 h. The reaction was cooled to 20° C., and a solution of KOH (10 g) in EtOH (100 mL) was added dropwise to make the pH 8-9. The mixture was stirred at 80° C. for another 2 h and cooled to 20° C., extracted with water (60 mL*1). The aqueous layer was concentrated to give the desired product (9.5 g, crude). LCMS (m/z): 232.9 [M+H]⁺

### Step 3: tert-butyl 3-bromo-2-methyl-6,7-dihydrothieno[3,2-c]pyridine-5(4H)-carboxylate

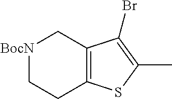 [SMILES: Cc1sc2c(c1Br)CN(C(=O)OC(C)(C)C)CC2]

To a solution of 3-bromo-2-methyl-4,5,6,7-tetrahydrothieno[3,2-c]pyridine (9.2 g, 39.5 mmol) and Et₃N (4.0 g, 39.5 mmol) in MeOH (50 mL) was added Boc₂O (8.6 g, 39.5 mmol) in 20 mL MeOH sat 0° C. The resulting solution was stirred at 20° C. for 16 h. after that, the solution was concentrated, and the residue was diluted with water (30 mL) and extracted with EA (20 mL×3). The organic layer was concentrated and the residue was purified by column chromatography (petroleum ether:ethyl acetate=10:1) to give the desired product (5.6 g, 42.7%). LCMS (m/z): 277.9 [M+H−56]⁺

### Step 4: tert-butyl 2,3-dimethyl-6,7-dihydrothieno[3,2-c]pyridine-5(4H)-carboxylate

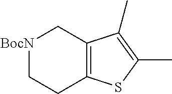 [SMILES: Cc1sc2c(c1C)CN(C(=O)OC(C)(C)C)CC2]

A mixture of tert-butyl 3-bromo-2-methyl-6,7-dihydrothieno[3,2-c]pyridine-5 (4H)-carboxylate (5.0 g, 15.0 mmol), methylboronic acid (2.7 g, 45.0 mmol), K₂CO₃ (4.1 g, 30.0 mmol) and Pd(dppf)Cl₂ (100 mg) in dioxane (100 mL) and H₂O (20 mL) was stirred at 100° C. for 4 h under N₂. The reaction solution was filtered, and the filtrate was concentrated. The residue was washed with water (30 mL) and extracted with EtOAc (20 mL×3). The organic layer was concentrated, and the residue was purified by column chromatography (petroleum ether:ethyl acetate=20:1) to give the desired product (2.0 g, 50%). LCMS (m/z): 212.1 [M+H−56]⁺

### Step 5: 2,3-dimethyl-4,5,6,7-tetrahydrothieno[3,2-c]pyridine

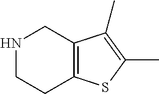

To a solution of tert-butyl 2,3-dimethyl-6,7-dihydrothieno[3,2-c]pyridine-5 (4H)-carboxylate (2.0 g, 7.5 mmol) in EtOAc (50 mL) was added dropwise EA.HCl (15 mL) at 0° C. The mixture was stirred at 18° C. for 4 h. The reaction solution was concentrated to give the desired product (1.5 g, crude). LCMS (m/z): 168.1 [M+H]⁺

### Step 6: (R)-2,3-dimethyl-5-(oxiran-2-ylmethyl)-4,5,6,7-tetrahydrothieno[3,2-c]pyridine

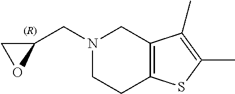

To a solution of 2,3-dimethyl-4,5,6,7-tetrahydrothieno[3,2-c]pyridine (1.25 g, 7.5 mmol) in DMF (20 mL) was added KF (1.8 g, 30.0 mmol) and (S)-oxiran-2-ylmethyl 3-nitrobenzenesulfonate (3.9 g, 15.0 mmol). The mixture was stirred at 18° C. under N₂ for 16 h. The reaction solution was used in next step. LCMS (m/z): 224.2 [M+H]⁺

### Step 7: (S)-1-amino-3-(2,3-dimethyl-6,7-dihydrothieno[3,2-c]pyridin-5(4H)-yl)propan-2-ol

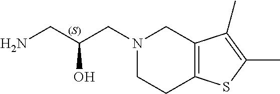

To a solution of (R)-2,3-dimethyl-5-(oxiran-2-ylmethyl)-4,5,6,7-tetrahydrothieno [3,2-c]pyridine (1.67 g, 7.5 mmol) in DMF (20 mL) and EtOH (20 mL) was added NH₃.H₂O (40 mL). The mixture was stirred at 100° C. for 4 h. The reaction solution was concentrated, and the residue was re-dissolved in MeOH (30 mL) and filtered. The filtrate was concentrated, residue was purified by column chromatography (DCM:MeOH=10:1) to give the desired product (1.1 g, 61.1%). LCMS (m/z): 241.1 [M+H]⁺

### Step 8: (S)-2-((1-acetylpiperidin-4-yl)amino)-N-(3-(2,3-dimethyl-6,7-dihydrothieno[3,2-c]pyridin-5(4H)-yl)-2-hydroxypropyl)isonicotinamide

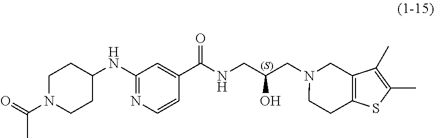

To a stirring solution of 2-((1-acetylpiperidin-4-yl)amino)isonicotinic acid (197 mg, 0.75 mmol) in DCM (20 mL) and Et₃N (127 mg, 1.25 mmol) was added HATU (356 mg, 0.94 mmol), the resulting solution was stirred at 15° C. for 30 min, (S)-1-amino-3-(2,3-dimethyl-6,7-dihydrothieno[3,2-c]pyridin-5(4H)-yl) propan-2-ol (150 mg, 0.63 mmol) in 2 mL DCM was then added. The mixture was stirred at 15° C. for another 16 h. Solution was concentrated, and the residue was purified by Pre-HPLC to give the title compound (40 mg, 13.3%). ¹HNMR (CD₃OD, 400 MHz) δ (ppm): 7.95 (d, J=5.5 Hz, 1H), 6.86 (s, 1H), 6.78-6.68 (m, 1H), 4.42 (d, J=13.6 Hz, 1H), 4.07 (quin, J=6.0 Hz, 1H), 4.02-3.84 (m, 2H), 3.56-3.39 (m, 4H), 3.30-3.21 (m, 1H), 2.96-2.75 (m, 5H), 2.74-2.62 (m, 2H), 2.27 (s, 3H), 2.12 (s, 3H), 2.09-1.98 (m, 2H), 1.92 (s, 3H), 1.52-1.31 (m, 2H). LCMS (m/z): 486.3 [M+H]⁺

### Example 29: (S)—N-(3-(6,7-dihydrothiazolo[5,4-c]pyridin-5(4H)-yl)-2-hydroxypropyl)-6-(oxetan-3-ylamino)pyrimidine-4-carboxamide (Compound 1-16)

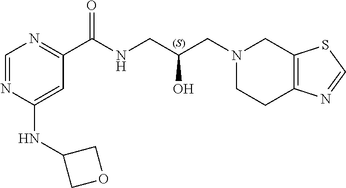

### Step 1: tert-butyl 3-bromo-4-oxopiperidine-1-carboxylate

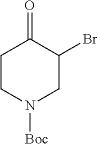 [SMILES: CC(C)(C)OC(=O)N1CCC(=O)C(Br)C1]

To the stirring solution of tert-butyl 4-oxopiperidine-1-carboxylate (10 g, 50 mmol) in DCM 100 mL, Br₂ was slowly added (8.0 g, 50 mmol) at 0° C. and the solution was stirred at 0° C. for 6 h or until the reaction complete by TLC analysis. Na₂SO₃ (aq.) was then added, the mixture was extracted with DCM, organic phase was washed with aq. NaHCO₃ and then separated. Boc₂O (10.9 g, 50 mmol) and TEA (7 ml) was then added, the resulting solution was stirred at 0° C. for a further 2 h, Solvents were then evaporated and the residue used directly to the next step without further purification (5.0 g, yield: 36.0%).

### Step 2: tert-butyl 2-amino-6,7-dihydrothiazolo[5,4-c]pyridine-5(4H)-carboxylate

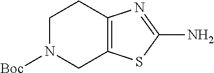 [SMILES: CC(C)(C)OC(=O)N1CCc2nc(N)sc2C1]

To the stirring solution of tert-butyl 3-bromo-4-oxopiperidine-1-carboxylate (5.0 g, 18 mmol) in DMF (50 mL) was added thiourea (1.37 g, 18 mmol), resulting solution was then heated at 120° C. for 3 h. The solvents were evaporated and the residue purified by column separation to afford desired product as pale yellow oil (2.2 g, yield: 47%). LCMS: 256.1 (M+1).

### Step 3: tert-butyl 2-bromo-6,7-dihydrothiazolo[5,4-c]pyridine-5(4H)-carboxylate

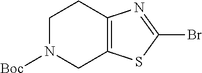 [SMILES: CC(C)(C)OC(=O)N1CCc2nc(Br)sc2C1]

To a stirring solution of tert-butyl 2-amino-6,7-dihydrothiazolo[5,4-c]pyridine-5(4H)-carboxylate (2.0 g, 7.84 mmol) in DCM (30 mL) was added tert-butyl nitrite (1.24 g, 12 mmol) and CuBr₂ (1.78 g, 8 mmol). The solution was stirred at 0° C. for 3 h. Once LCMS showed the reaction to be complete, solvents were then evaporated and the residue was purified with column separation to afford desired product as white solid (1.1 g, yield: 44%); LCMS: 318.9/320.9 (M+1).

### Step 3: tert-butyl 6,7-dihydrothiazolo[5,4-c]pyridine-5(4H)-carboxylate

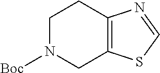 [SMILES: CC(C)(C)OC(=O)N1CCc2ncsc2C1]

To the solution of tert-butyl 2-bromo-6,7-dihydrothiazolo[5,4-c]pyridine-5(4H)-carboxylate (1.1 g, 3.45 mmol) in methanol (20 mL) was added wet Pd/C (100 mg) and potassium hydroxide (280 mg, 5 mmol), the resulting mixture was then hydrogenated under H₂ with a balloon for 1 h, TLC and LCMS showed the reaction completed. The mixture was filtered, the filtrated was collected and solvent was evaporated to dryness. The residue was then re-dissolved in DCM, washed with water, dried and concentrated to afford desired product as colorless oil (800 mg, yield: 96.6%). LCMS: 241.1 (M+1).

### Step 4: 4,5,6,7-tetrahydrothiazolo[5,4-c]pyridine

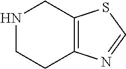

To a solution of tert-butyl 6,7-dihydrothiazolo[5,4-c]pyridine-5(4H)-carboxylate (1.5 g, 6.3 mmol) in EA (30 mL) was added 4N HCl/EA (5 mL) at 0° C., the resulting solution was stirred at 25° C. for 12 h. Solvent was evaporated to give the crude product (900 mg, 102% yield).

### Step 5: (R)-5-(oxiran-2-ylmethyl)-4,5,6,7-tetrahydrothiazolo[5,4-c]pyridine

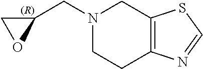

To the stirring solution of 4,5,6,7-tetrahydrothiazolo[5,4-c]pyridine (882 mg, 6.3 mmol) in THF (50 mL) and TEA (1.2 g, 12.6 mmol), was added (S)-oxiran-2-ylmethyl 3-nitrobenzenesulfonate(1.9 g, 7.6 mmol) and KF (1.4 g, 25.2 mmol), the resulting mixture was stirred at 25° C. for 16 h. The solid was filtered off and the filtrate concentrated to give the crude product (1.3 g, 108% yield), which was used directly to the next step without further purification. LCMS (m/z): 197.1 (M+1).

### Step 6: (S)-1-amino-3-(6,7-dihydrothiazolo[5,4-c]pyridin-5(4H)-yl)propan-2-ol

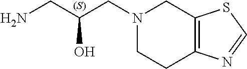

To a solution of (R)-5-(oxiran-2-ylmethyl)-4,5,6,7-tetrahydrothiazolo[5,4-c]pyridine (1.3 g, 6.6 mmol) in EtOH (100 mL) was added NH₃.H₂O (50 mL), solution was stirred at 25° C. for 16 h. After the reaction was cooled to room temperature, the solvent was removed to give the crude product which was then purified by chromatography on silica gel to give the desired product (350 mg, 25% yield). LCMS (m/z): 214.1 (M+1).

### Step 7: (S)-6-chloro-N-(3-(6,7-dihydrothiazolo[5,4-c]pyridin-5(4H)-yl)-2-hydroxypropyl)pyrimidine-4-carboxamide

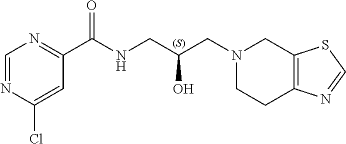

To a stirring solution of (S)-1-amino-3-(6,7-dihydrothiazolo[5,4-c]pyridin-5(4H)-yl)propan-2-ol (250 mg, 1.17 mmol) in DCM (50 mL) and TEA (1 mL) was added 6-chloropyrimidine-4-carbonyl chloride (207 mg, 1.17 mmol) at 0° C. The solution was then stirred at 25° C. for 4 h after which, the mixture was poured into 50 mL ice-water and extracted with DCM (50 mL×3), organic phase was combined and dried over Na₂SO₄. Solvent was then evaporated and residue purified by preperative-TLC separation to give the desired product (240 mg, 58% yield); LCMS (m/z): 354.1 (M+1).

### Step 8: (S)—N-(3-(6,7-dihydrothiazolo[5,4-c]pyridin-5(4H)-yl)-2-hydroxypropyl)-6-(oxetan-3-ylamino)pyrimidine-4-carboxamide

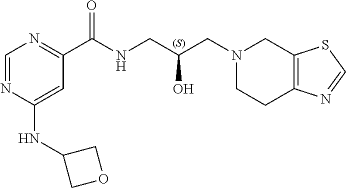

A mixture of (S)-6-chloro-N-(3-(6,7-dihydrothiazolo[5,4-c]pyridin-5(4H)-yl)-2-hydroxypropyl) pyrimidine-4-carboxamide (80 mg, 0.23 mmol), oxetan-3-amine (33 mg, 0.46 mmol) and TEA (0.1 mL) in i-PrOH (10 mL) was stirred at 80° C. for 12 h. TLC showed the reaction completed and solvent was evaporated to dryness. The residue was then purified by HPLC separation to afford the desired product (45 mg, 50% yield). 1H NMR (400 MHz, METHANOL-d₄) δ (ppm): 8.85 (s, 1H), 8.34 (s, 1H), 7.15 (br. s., 1H), 5.12 (br. s., 1H), 4.97 (t, J=6.9 Hz, 2H), 4.62 (t, J=6.3 Hz, 2H), 4.05 (quin, J=5.9 Hz, 1H), 3.88 (s, 2H), 3.61-3.54 (m, 1H), 3.53-3.45 (m, 1H), 3.05-2.98 (m, 2H), 2.95 (d, J=5.5 Hz, 2H), 2.78-2.70 (m, 2H); LCMS (m/z): 391.2 (M+1).

### Example 30: (S)-6-((1-acetylazetidin-3-yl)amino)-N-(3-(6,7-dihydrothiazolo[5,4-c]pyridin-5(4H)-yl)-2-hydroxypropyl)pyrimidine-4-carboxamide (Compound 1-17)

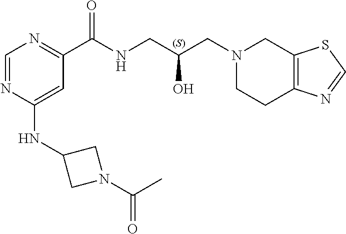

A mixture of (S)-6-chloro-N-(3-(6,7-dihydrothiazolo[5,4-c]pyridin-5(4H)-yl)-2-hydroxypropyl) pyrimidine-4-carboxamide (80 mg, 0.23 mmol), 1-(3-aminoazetidin-1-yl)ethanone (52 mg, 0.46 mmol) and TEA (0.1 mL) in i-PrOH (10 mL) was stirred at 80° C. for 12 h or until shown to be complete by TLC analysis. The solvent was evaporated to dryness and the residue was then purified by HPLC separation to afford the desired product (24 mg, 24% yield). 1H NMR (400 MHz, METHANOL-d4) δ (ppm): 8.86 (s, 1H), 8.38 (s, 1H), 7.16 (br. s., 1H), 4.77 (br. s., 1H), 4.59 (t, J=8.4 Hz, 1H), 4.43-4.31 (m, 1H), 4.13-4.02 (m, 2H), 3.95-3.85 (m, 3H), 3.62-3.49 (m, 2H), 3.05-2.91 (m, 4H), 2.74 (d, J=5.8 Hz, 2H), 1.91 (s, 3H); LCMS (m/z): 432.2 (M+1).

### Example 31: (S)-6-((1-acetylpiperidin-4-yl)amino)-N-(3-(6,7-dihydrothiazolo[5,4-c]pyridin-5(4H)-yl)-2-hydroxypropyl)pyrimidine-4-carboxamide (Compound 1-18)

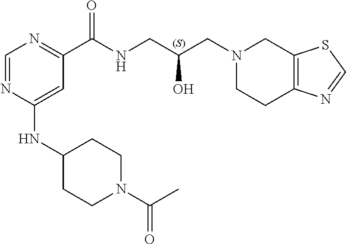

A mixture of (S)-6-chloro-N-(3-(6,7-dihydrothiazolo[5,4-c]pyridin-5(4H)-yl)-2-hydroxypropyl) pyrimidine-4-carboxamide (80 mg, 0.23 mmol), 1-(4-aminopiperidin-1-yl)ethanone (65 mg, 0.46 mmol) and TEA (0.1 mL) in i-PrOH (10 mL) was stirred at 80° C. for 12 h. TLC showed the reaction completed and solvent was evaporated to dryness, residue was then purified by Pre-HPLC separation to afford the desired product (29 mg, 27% yield). ¹H NMR (400 MHz, METHANOL-d4) δ (ppm): 8.86 (s, 1H), 8.34 (s, 1H), 7.10 (s, 1H), 4.45 (d, J=13.6 Hz, 1H), 4.18 (br. s., 1H), 4.05 (quin, J=6.0 Hz, 1H), 3.95 (d, J=13.8 Hz, 1H), 3.88 (s, 2H), 3.60-3.54 (m, 1H), 3.53-3.45 (m, 1H), 3.32-3.22 (m, 1H), 3.06-2.98 (m, 2H), 2.96 (d, J=5.8 Hz, 2H), 2.93-2.86 (m, 1H), 2.74 (d, J=6.0 Hz, 2H), 2.14 (s, 3H), 2.10 (d, J=13.8 Hz, 1H), 2.02 (d, J=11.0 Hz, 1H), 1.56-1.48 (m, 1H), 1.47-1.38 (m, 1H), 1.10-1.13 (m, 1H); LCMS (m/z): 460.2 (M+1).

### Example 32: (S)-2-((1-acetylpiperidin-4-yl)amino)-N-(3-(6,7-dihydrothiazolo[5,4-c]pyridin-5(4H)-yl)-2-hydroxypropyl)isonicotinamide (Compound 1-19)

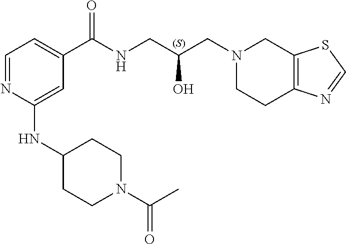

To a stirring solution of 2-((1-acetylpiperidin-4-yl)amino)isonicotinic acid (100 mg, 0.35 mmol) in DCM (10 mL) was added HATU (160 mg, 0.4 mmol) and TEA (1 mL) at 20° C., the resulting solution was then stirred at this temperature for 10 min, (S)-1-amino-3-(6,7-dihydrothiazolo[5,4-c]pyridin-5(4H)-yl)propan-2-ol (75 mg, 0.35 mmol) was then added and solution was stirred for another 12 h. TLC showed the reaction completed and solvent was evaporated to dryness, residue was then purified by Pre-HPLC separation to afford the desired product (72 mg, 45% yield). 1H NMR (400 MHz, METHANOL-d4) δ (ppm): 9.07 (s, 1H), 8.00 (d, J=6.5 Hz, 1H), 7.49 (s, 1H), 7.22 (dd, J=1.4, 6.7 Hz, 1H), 4.76 (br. s., 2H), 4.54 (d, J=14.3 Hz, 1H), 4.38 (d, J=6.0 Hz, 1H), 4.03 (d, J=14.3 Hz, 1H), 3.98-3.90 (m, 1H), 3.83 (br. s., 2H), 3.64-3.56 (m, 1H), 3.54-3.44 (m, 2H), 3.43-3.34 (m, 2H), 3.31-3.23 (m, 2H), 2.97-2.83 (m, 2H), 2.20-2.05 (m, 5H), 1.72-1.59 (m, 1H), 1.57-1.45 (m, 1H); LCMS (m/z): 459.1 (M+1).

### Example 33: (S)—N-(2-hydroxy-3-(2-methyl-6,7-dihydrothiazolo[5,4-c]pyridin-5(4H)-yl)propyl)-6-(oxetan-3-ylamino)pyrimidine-4-carboxamide (Compound 1-8)

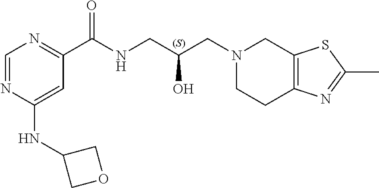

### Step 1: benzyl 3-bromo-4-oxo-piperidine-1-carboxylate

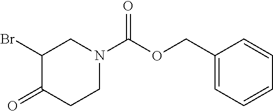 [SMILES: O=C1CCN(C(=O)OCc2ccccc2)CC1Br]

To a solution of benzyl 4-oxo-piperidine-1-carboxylate (3.00 g, 12.9 mmol) in chloroform (30 mL) was added bromine (0.7 mL) at 0° C. After stirring for 12 h at 15-20° C., water was added, mixture was extracted with DCM, and the organic layer was combined and washed with brine, dried over anhydrous sodium sulfate, and then concentrated to give the crude product (3.16 g, 79% yield).

### Step 2: benzyl 2-methyl-6,7-dihydro-4H-thiazolo[5,4-c]pyridine-5-carboxylate

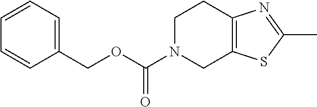 [SMILES: Cc1nc2c(s1)CN(C(=O)OCc1ccccc1)CC2]

To a solution of benzyl 3-bromo-4-oxo-piperidine-1-carboxylate (3.16 g, 10 mmol) in DMF (15 mL) was added thioacetamide (2.95 g, 38.8 mmol). The resulting mixture was then heated at 100° C. for 1.5 hour, and diluted with 200 mL water, extracted with EA (50 mL×3). The organic layer was combined and washed with brine, dried over anhydrous sodium sulfate, and then concentrated. The residue was purified by silica gel column chromatography (chloroform/methanol, 97:3) to get the title compound (1.65 g, 57% yield) as colorless oil. LCMS (m/z): 289.1 (M+1).

### Step 3: 2-methyl-4,5,6,7-tetrahydrothiazolo[5,4-c]pyridine hydrobromide

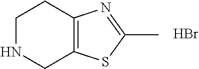

Benzyl 2-methyl-6,7-dihydro-4H-thiazolo[5,4-c]pyridine-5-carboxylate (1.65 g, 5.7 mmol) was dissolved in 25% hydrogen bromide/acetic acid (10.0 mL). After stirring at room temperature for 30 minutes, the precipitated solid was collected by filtration and washed with methanol. The title compound (1.02 g, 77% yield) was obtained as a light yellow solid. LCMS (m/z): 155.2 (M+1).

### Step 4: (R)-2-methyl-5-(oxiran-2-ylmethyl)-4,5,6,7-tetrahydrothiazolo[5,4-c]pyridine

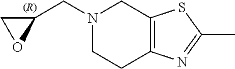

A stirring solution of 2-methyl-4,5,6,7-tetrahydrothiazolo[5,4-c]pyridine (550 mg, 3.57 mmol) in THF (50 mL) was added (S)-oxiran-2-ylmethyl 3-nitrobenzenesulfonate(1.1 g, 4.3 mmol), TEA (721 mg, 7.14 mmol) and KF (421 mg, 7.14 mmol). The resulting mixture was stirred at 25° C. for 16 h. Precipitate was filtered off and the filtrate was concentrated to give the crude product (800 mg, 108% yield), which was used directly in the next step without further purification.

### Step 5: (S)-1-amino-3-(2-methyl-6,7-dihydrothiazolo[5,4-c]pyridin-5(4H)-yl)propan-2-ol

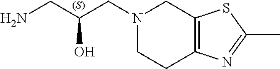

To a solution of (R)-2-methyl-5-(oxiran-2-ylmethyl)-4,5,6,7-tetrahydrothiazolo[5,4-c]pyridine (800 mg, 3.8 mmol) in EtOH (100 mL) was added NH₃.H₂O (50 mL) and was stirred at 25° C. for 16 h. After the reaction was cooled to room temperature, the solvent was removed by concentration under vacuum and the residue was purified by chromatography on silica gel to give the desired product (300 mg, 34.8% yield). LCMS (m/z): 228.2 (M+1).

### Step 6: (S)-6-chloro-N-(2-hydroxy-3-(2-methyl-6,7-dihydrothiazolo[5,4-c]pyridin-5(4H)-yl)propyl)pyrimidine-4-carboxamide

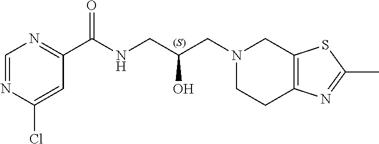

To the stirring solution of (S)-1-amino-3-(2-methyl-6,7-dihydrothiazolo[5,4-c]pyridin-5(4H)-yl)propan-2-ol (200 mg, 0.88 mmol) in DCM (50 mL) and TEA (1 mL) was added the solution of 6-chloropyrimidine-4-carbonyl chloride (177 mg, 1.0 mmol, in 2 mL DCM) at 0° C., the resulting solution was stirred at 25° C. for 4 h. After that, the mixture was poured into 50 mL ice-water, extracted with DCM (50 mL×3). The organic phase was combined and dried over anhydrous Na₂SO₄. The solvent was removed by vacuum to give the crude product which was purified by preperative-TLC separation to give the desired product (300 mg, 93% yield); LCMS (m/z): 368.1 (M+1).

### Step 7: (S)—N-(2-hydroxy-3-(2-methyl-6,7-dihydrothiazolo[5,4-c]pyridin-5(4H)-yl)propyl)-6-(oxetan-3-ylamino)pyrimidine-4-carboxamide

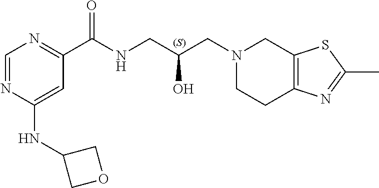

A mixture of (S)-6-chloro-N-(2-hydroxy-3-(2-methyl-6,7-dihydrothiazolo[5,4-c]pyridin-5(4H)-yl)propyl)pyrimidine-4-carboxamide (100 mg, 0.27 mmol), oxetan-3-amine (39 mg, 0.54 mmol) and TEA (0.1 mL) in i-PrOH (10 mL) was stirred at 80° C. for 12 h or until shown to be complete by TLC analysis. The solvent was removed and the residue was then purified by HPLC separation to give the desired product (17 mg, 15.6% yield). 1H NMR (400 MHz, METHANOL-d4) δ (ppm): 8.36 (s, 1H), 7.15 (br. s., 1H), 5.13 (br. s., 1H), 4.98 (t, J=6.9 Hz, 2H), 4.62 (t, J=6.3 Hz, 2H), 4.04 (t, J=5.8 Hz, 1H), 3.78 (s, 2H), 3.53 (dq, J=5.8, 13.6 Hz, 2H), 3.01-2.93 (m, 2H), 2.85 (d, J=5.5 Hz, 2H), 2.71 (d, J=5.8 Hz, 2H), 2.66 (s, 3H); LCMS (m/z): 405.2 (M+1).

### Example 34: (S)-6-((1-acetylazetidin-3-yl)amino)-N-(2-hydroxy-3-(2-methyl-6,7-dihydrothiazolo[5,4-c]pyridin-5(4H)-yl)propyl)pyrimidine-4-carboxamide (Compound 1-6)

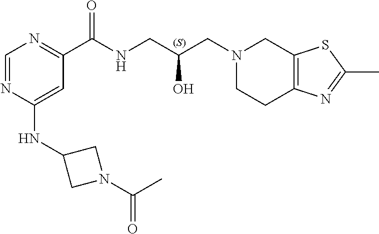

A mixture of (S)-6-chloro-N-(2-hydroxy-3-(2-methyl-6,7-dihydrothiazolo[5,4-c]pyridin-5(4H)-yl)propyl)pyrimidine-4-carboxamide (100 mg, 0.27 mmol), 1-(3-aminoazetidin-1-yl)ethanone (61 mg, 0.54 mmol) and TEA (0.1 mL) in i-PrOH (10 mL) was stirred at 80° C. for 12 h. TLC showed the reaction completed and solvent was removed, residue was then purified by HPLC separation to give the desired product (18 mg, 15% yield). ¹H NMR (400 MHz, METHANOL-d4) δ (ppm): 8.40 (s, 1H), 7.16 (br. s., 1H), 4.77 (br. s., 1H), 4.59 (t, J=8.4 Hz, 1H), 4.36 (t, J=9.0 Hz, 1H), 4.10 (dd, J=5.0, 9.0 Hz, 1H), 4.04 (t, J=5.9 Hz, 1H), 3.90 (dd, J=5.0, 10.3 Hz, 1H), 3.60-3.48 (m, 2H), 3.01-2.92 (m, 2H), 2.85 (t, J=5.6 Hz, 2H), 2.71 (d, J=6.3 Hz, 2H), 2.66 (s, 3H), 1.91 (s, 3H); LCMS (m/z): 446.2 (M+1).

### Example 35: (S)-6-((1-acetylpiperidin-4-yl)amino)-N-(2-hydroxy-3-(2-methyl-6,7-dihydrothiazolo[5,4-c]pyridin-5(4H)-yl)propyl)pyrimidine-4-carboxamide (Compound 1-7)

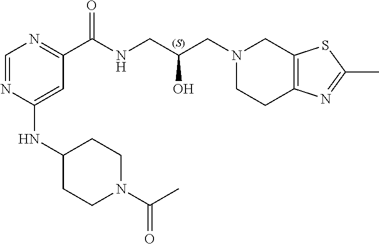

A mixture of (S)-6-chloro-N-(2-hydroxy-3-(2-methyl-6,7-dihydrothiazolo[5,4-c]pyridin-5(4H)-yl)propyl)pyrimidine-4-carboxamide (100 mg, 0.27 mmol), 1-(4-aminopiperidin-1-yl)ethanone (76 mg, 0.54 mmol) and TEA (0.1 mL) in i-PrOH (10 mL) was stirred at 80° C. for 12 h. TLC showed the reaction completed and solvent was removed and residue was then purified by Pre-HPLC separation to give the desired product (21 mg, 16% yield). ¹H NMR (400 MHz, METHANOL-d4) δ (ppm): 8.35 (s, 1H), 7.10 (s, 1H), 4.45 (d, J=13.3 Hz, 1H), 4.18 (br. s., 1H), 4.04 (quin, J=5.9 Hz, 1H), 3.95 (d, J=13.6 Hz, 1H), 3.78 (s, 2H), 3.58-3.47 (m, 2H), 3.03-2.83 (m, 6H), 2.71 (d, J=6.0 Hz, 2H), 2.66 (s, 3H), 2.14 (s, 3H), 2.10 (d, J=13.6 Hz, 1H), 2.03 (d, J=11.0 Hz, 1H), 1.56-1.48 (m, 1H), 1.47-1.38 (m, 1H); LCMS (m/z): 474.3 (M+1).

### Example 36: (S)—N-(3-(2-amino-6,7-dihydrothiazolo[4,5-c]pyridin-5(4H)-yl)-2-hydroxypropyl)-6-(oxetan-3-ylamino)pyrimidine-4-carboxamide (Compound 1-20)

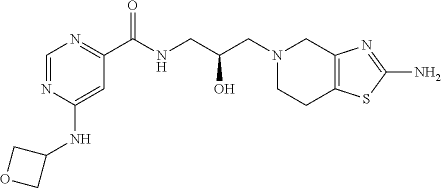 [SMILES: Nc1nc2c(s1)CCN(C[C@@H](O)CNC(=O)c1cc(NC3COC3)ncn1)C2]

### Step 1: tert-butyl 2-(((allyloxy)carbonyl)amino)-6,7-dihydrothiazolo[4,5-c]pyridine-5(4H)-carboxylate

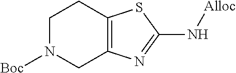

To a stirring solution of tert-butyl 2-amino-6,7-dihydrothiazolo[4,5-c]pyridine-5(4H)-carboxylate (3.0 g, 11.76 mmol) in DCM (30 mL) and pyridine (10 mL) was added AllocCl (2.84 g, 23.53 mmol) at 0° C. The reacting solution was stirred at 50° C. for 16 h. LCMS showed the reaction worked well, the mixture was then taken up with DCM and washed with H₂O, and the DCM layer was evaporated and the residue was purified by silica column (DCM/MeOH=50:1) to give the tert-butyl 2-(((allyloxy)carbonyl)amino)-6,7-dihydrothiazolo[4,5-c]pyridine-5(4H)-carboxylate (3.3 g, yield: 82.7%). ¹H NMR (400 MHz, CDCl₃-d): δ (ppm): 6.10-5.96 (m, 1H), 5.45-5.26 (m, 2H), 4.79 (d, J=6.0 Hz, 2H), 4.50 (br. s., 2H), 3.72 (br. s., 2H), 2.77 (br. s., 2H), 1.52-1.46 (m, 9H).

### Step 2: allyl (4,5,6,7-tetrahydrothiazolo[4,5-c]pyridin-2-yl)carbamate

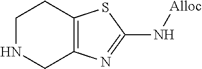

To a solution of tert-butyl 2-(((allyloxy)carbonyl)amino)-6,7-dihydrothiazolo [4,5-c]pyridine-5(4H)-carboxylate (3.3 g, 9.73 mmol) in DCM (40 mL) was added TFA (8 mL). The mixture was stirred at 25° C. for 3 h. TLC showed the reaction completed. Solvent was then evaporated to give the desired product (2.33 g crude, yield: 100%) which was used directly to the next step without further purification.

### Step 3: (R)-allyl (5-(oxiran-2-ylmethyl)-4,5,6,7-tetrahydrothiazolo[4,5-c]pyridin-2-yl)carbamate

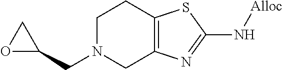

To a solution of allyl (4,5,6,7-tetrahydrothiazolo[4,5-c]pyridin-2-yl)carbamate (2.33 g, 9.75 mmol) in THF (50 mL) was added (S)-oxiran-2-ylmethyl 3-nitrobenzenesulfonate (3.03 g, 11.70 mmol) and KF (2.83 g, 48.75 mmol). The mixture was stirred at 25° C. for 16 hours. The reaction mixture was filtered and the mixture was used directly for the next step.

### Step 4: (S)-allyl (5-(3-amino-2-hydroxypropyl)-4,5,6,7-tetrahydrothiazolo[4,5-c]pyridin-2-yl) carbamate

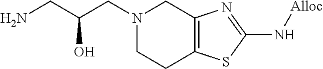

(R)-allyl(5-(oxiran-2-ylmethyl)-4,5,6,7-tetrahydrothiazolo[4,5-c]pyridin-2-yl)carbamate (9.75 mmol) was dissolved in NH₃.H₂O (30 mL) and EtOH (30 mL). The mixture was stirred at 25° C. for 16 h. Solvent was evaporated, residue was purified with column separation to give the desired product (1.2 g, yield: 40% two steps), LCMS (m/z): 313.1 [M+H]⁺

### Step 5: (S)-allyl (5-(3-(6-chloropyrimidine-4-carboxamido)-2-hydroxypropyl)-4,5,6,7-tetrahydrothiazolo[4,5-c]pyridin-2-yl)carbamate

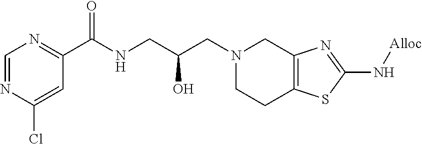

To a stirring solution of (S)-allyl (5-(3-amino-2-hydroxypropyl)-4,5,6,7-tetrahydrothiazolo[4,5-c]pyridin-2-yl) carbamate (2.0 g, 6.41 mmol) in DCM (20 mL) was added TEA (1.29 g, 12.82 mmol), solution was cooled to 0° C. and 6-chloropyrimidine-4-carbonyl chloride (1.13 g, 6.41 mmol) was added in 10 min. The mixture was stirred at 25° C. for 1 h, and taken up with DCM, washed with H₂O. The DCM layer was combined and evaporated, the residue was purified by silica column (DCM/MeOH=20:1) to give the desired product. (1.0 g, yield: 34.5%). LCMS (m/z): 453.1 [M+H]⁺.

### Step 7: (S)—N-(3-(2-amino-6,7-dihydrothiazolo[4,5-c]pyridin-5(4H)-yl)-2-hydroxypropyl)-6-(oxetan-3-ylamino)pyrimidine-4-carboxamide

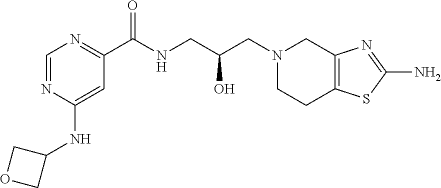 [SMILES: Nc1nc2c(s1)CCN(C[C@@H](O)CNC(=O)c1cc(NC3COC3)ncn1)C2]

To a solution of (S)-allyl (5-(3-(6-chloropyrimidine-4-carboxamido)-2-hydroxypropyl)-4,5,6,7-tetrahydrothiazolo[4,5-c]pyridin-2-yl)carbamate (300 mg, 0.664 mmol) in i-PrOH (5 mL) was added oxetan-3-amine (47.8 mg, 0.664 mmol) and TEA (134.1 mg, 1.33 mmol). The mixture was stirred at 80° C. for 16 hours. TLC showed the reaction completed, and solvent was evaporated, the residue was purified by prep-TLC (DCM/MeOH=10:1) to give the (S)-allyl (5-(2-hydroxy-3-(6-(oxetan-3-ylamino)pyrimidine-4-carboxamido)propyl)-4,5,6,7-tetrahydrothiazolo[4,5-c]pyridin-2-yl)carbamate (70 mg, yield: 21.6%). The (S)-allyl (5-(2-hydroxy-3-(6-(oxetan-3-ylamino)pyrimidine-4-carboxamido)propyl)-4,5,6,7-tetrahydrothiazolo[4,5-c]pyridin-2-yl)carbamate (70 mg, 0.143 mmol), 1,3-dimethylpyrimidine-2,4,6(1H,3H,5H)-trione (111.7 mg, 0.716 mmol), Pd(PPh₃)₄ (33.0 mg, 0.03 mmol) were dissolved in THF (10 mL). The mixture was stirred at 25° C. for 16 hours under N₂, LCMS showed the reaction completed and the solution was evaporated and the residue was purified by prep-HPLC to give the target title compound. (50.8 mg, yield: 87.7%). ¹H NMR (400 MHz, CD₃OD-d₄): δ (ppm): 8.40 (s, 1H), 7.14 (br. s., 1H), 5.11 (br. s., 1H), 4.96 (t, J=6.8 Hz, 2H), 4.60 (t, J=6.3 Hz, 2H), 4.04-3.97 (m, 1H), 3.54-3.44 (m, 4H), 2.86 (qd, J=5.7, 11.0 Hz, 2H), 2.66 (d, J=6.0 Hz, 4H). LCMS (m/z): 406.2 [M+H]⁺

### Example 37: S)—N-(2-hydroxy-3-(2-methyl-6,7-dihydrooxazolo[4,5-c]pyridin-5(4H)-yl)propyl)-6-(oxetan-3-ylamino)pyrimidine-4-carboxamide (Compound 4-1)

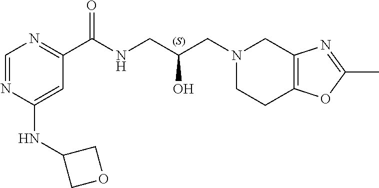

### Step 1: Benzyl 4-(hydroxyimino)piperidine-1-carboxylate

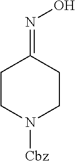 [SMILES: O=C(OCc1ccccc1)N1CCC(=NO)CC1]

A mixture of benzyl 4-oxopiperidine-1-carboxylate (10 g, 43 mmol), hydroxylamine hydrochloride (5.9 g, 86 mmol) and sodium acetate (7.4 g, 90 mmol) in EtOH (200 mL) was stirred at 80° C. for 3 h. The solvent was removed and mixture was diluted with water (100 mL), then extracted with EA (50 mL×3). The organic phase was combined and dried over Na₂SO₄. Solvent was then removed to give the crude product (10.5 g, 99% yield) which was used without further purification.

### Step 2: Benzyl 4-((tosyloxy)imino)piperidine-1-carboxylate

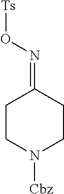 [SMILES: Cc1ccc(S(=O)(=O)ON=C2CCN(C(=O)OCc3ccccc3)CC2)cc1]

To the stirring solution of benzyl 4-(hydroxyimino) piperidine-1-carboxylate (10.5 g, 43 mmol) in DCM (200 mL) and TEA (8.6 g, 86 mmol) was added TsCl (9.8 g, 51.6 mmol) in portions at 0° C. and the resulting solution heated at 40° C. for 3 h. Solution was then diluted with water and washed with aq. NaHCO₃. The organic layer was combined and dried, solvent was removed by concentration and the residue was purified by chromatography on silica gel to give the desired product (16 g, 93% yield).

### Step 3: benzyl 3-amino-4-oxopiperidine-1-carboxylate hydrochloride

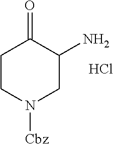 [SMILES: NC1CN(C(=O)OCc2ccccc2)CCC1=O ; Cl]

To the stirring solution of t-BuOK (3.6 g, 32 mmol) in EtOH (30 mL) and toluene (120 mL) was added benzyl 4-((tosyloxy)imino)piperidine-1-carboxylate (8 g, 20 mmol) slowly at 0° C., the resulting mixture was stirred at this temperature for 2 h. After that the temperature was allowed to warm to 25° C. and stirred for another 3 h. Then the mixture was next cooled to 0° C. and 5 mL HCl (aq) was added and the solution was stirred at 25° C. for 3 h. The solid was precipitated and collected by filtration to give the desired product 1.5 g, 18.6% yield). LCMS (m/z): 249.2 (M+1).

### Step 4: Benzyl 3-acetamido-4-oxopiperidine-1-carboxylate

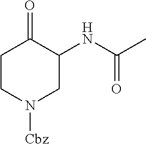 [SMILES: CC(=O)NC1CN(C(=O)OCc2ccccc2)CCC1=O]

To the stirring solution of benzyl 3-amino-4-oxopiperidine-1-carboxylate hydrochloride (1.5 g, 5.2 mmol) in DCM (20 mL) and TEA (1.1 g, 10.4 mmol) was added acetic anhydride (1.0 g, 10.4 mmol) at 0° C., the resulting solution was stirred at this temperature for 3 h. The solvent was removed by concentration and the crude was purified by chromatography on silica gel to give the desired product (1.5 g, 100% yield). LCMS (m/z): 291.2 (M+1).

### Step 5: benzyl 2-methyl-6,7-dihydrooxazolo[4,5-c]pyridine-5(4H)-carboxylate

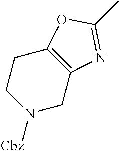 [SMILES: Cc1nc2c(o1)CCN(C(=O)OCc1ccccc1)C2]

A mixture of benzyl 3-acetamido-4-oxopiperidine-1-carboxylate (1.5 g, 5.2 mmol), Burgess reagent (2.5 g, 10.4 mmol) in THF (10 mL) was stirred at 120° C. for 30 min. The solvent was removed by concentration and the crude product was purified by chromatography on silica gel to give the desired product (0.8 g, 57% yield). 1H NMR (400 MHz, METHANOL-d₄ δ (ppm): 7.37-7.30 (m, 5H), 5.18-5.16 (m, 2H), 4.40 (br. s., 2H), 3.81 (t, J=5.8 Hz, 2H), 2.71 (br. s., 2H), 2.42 (s, 3H); LCMS (m/z): 273.1 (M+1).

### Step 6: 2-methyl-4,5,6,7-tetrahydrooxazolo[4,5-c]pyridine

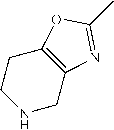

To a solution of benzyl 2-methyl-6,7-dihydrooxazolo[4,5-c]pyridine-5(4H)-carboxylate (0.8 g, 2.9 mmol) in MeOH (30 mL) was added Pd/C (100 mg) under N₂ atmosphere, then the mixture was stirred under hydrogen atmosphere (20 Psi) for 14 h. The catalyst was filter off and the filtrate was concentrated to give the desired product (400 mg, 100% yield). LCMS (m/z): 139.1 (M+1).

### Step 7: (R)-2-methyl-5-(oxiran-2-ylmethyl)-4,5,6,7-tetrahydrooxazolo[4,5-c]pyridine

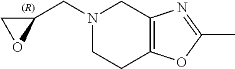

To the solution of 2-methyl-4,5,6,7-tetrahydrooxazolo[4,5-c]pyridine (400 mg, 2.9 mmol) in THF (50 mL) was added (S)-oxiran-2-ylmethyl 3-nitrobenzenesulfonate (0.9 g, 3.5 mmol) and KF (672 mg, 11.6 mmol), the resulting mixture was stirred at 25° C. for 16 h. The solid was filter off and the filtrate was concentrated to give the crude product (600 mg, 107% yield), which was used directly in the next step without further purification. LCMS (m/z): 195.1 (M+1).

### Step 8: (S)-1-amino-3-(2-methyl-6,7-dihydrooxazolo[4,5-c]pyridin-5(4H)-yl)propan-2-ol

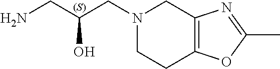

To a solution of (R)-2-methyl-5-(oxiran-2-ylmethyl)-4,5,6,7-tetrahydrooxazolo[4,5-c]pyridine (410 mg, 3.0 mmol) in EtOH (50 mL) was added NH₃.H₂O (50 mL), and the solution was stirred at 45° C. for 16 h. After the reaction mixture was cooled to room temperature and the solvent was removed by concentration, the residue was purified by chromatography on silica gel (DCM:MeOH=10:1) to give the crude product (450 mg, 74% yield). LCMS (m/z): 121.2 (M+1).

### Step 9: (S)-6-chloro-N-(2-hydroxy-3-(2-methyl-6,7-dihydrooxazolo[4,5-c]pyridin-5(4H)-yl)propyl)pyrimidine-4-carboxamide

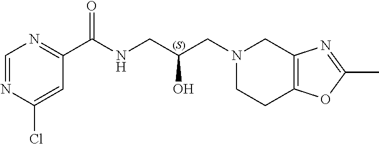

To a stirring solution of (S)-1-amino-3-(2-methyl-6,7-dihydrooxazolo[4,5-c]pyridin-5(4H)-yl)propan-2-ol (350 mg, 1.66 mmol) in DCM (50 mL) and TEA (1 mL) was added 6-chloropyrimidine-4-carbonyl chloride (293 mg, 1.66 mmol) at 0° C., the resulting solution was stirred at 25° C. for 4 h. After the reaction, the mixture was poured into 50 mL ice-water, extracted with DCM (50 mL×3) and dried over Na₂SO₄. Solvent was removed by concentration to give the crude product and then purified by preparative TLC separation to give the desired product (270 mg, 48.6% yield); LCMS (m/z): 352.1 (M+1).

### Step 10: (S)—N-(2-hydroxy-3-(2-methyl-6,7-dihydrooxazolo[4,5-c]pyridin-5(4H)-yl)propyl)-6-(oxetan-3-ylamino)pyrimidine-4-carboxamide

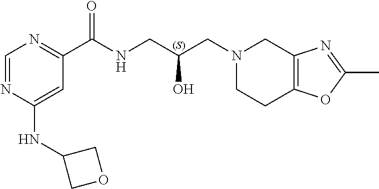

A mixture of (S)-6-chloro-N-(2-hydroxy-3-(2-methyl-6,7-dihydrooxazolo[4,5-c]pyridin-5(4H)-yl)propyl)pyrimidine-4-carboxamide (90 mg, 0.25 mmol), oxetan-3-amine (36 mg, 0.5 mmol) and TEA (0.1 mL) in i-PrOH (10 mL) was stirred at 80° C. for 12 h or until the reaction was complete by TLC analysis. The solvent was removed under vacuum, residue was then purified by HPLC separation to give the desired product (34 mg, 35% yield). ¹H NMR (400 MHz, METHANOL-d4) δ (ppm): 8.40 (s, 1H), 7.16 (br. s., 1H), 5.13 (br. s., 1H), 4.98 (t, J=6.9 Hz, 2H), 4.62 (t, J=6.3 Hz, 2H), 4.03 (t, J=5.8 Hz, 1H), 3.61-3.53 (m, 1H), 3.53-3.42 (m, 3H), 3.01-2.91 (m, 2H), 2.74 (t, J=5.3 Hz, 2H), 2.70 (d, J=6.0 Hz, 2H), 2.42 (s, 3H); LCMS (m/z): 389.2 (M+1).

### Example 38: (S)-6-((1-acetylazetidin-3-yl)amino)-N-(2-hydroxy-3-(2-methyl-6,7-dihydrooxazolo[4,5-c]pyridin-5(4H)-yl)propyl)pyrimidine-4-carboxamide (Compound

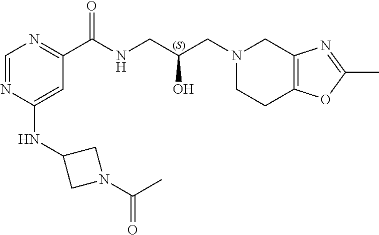

A mixture of (S)-6-chloro-N-(2-hydroxy-3-(2-methyl-6,7-dihydrooxazolo[4,5-c]pyridin-5(4H)-yl)propyl)pyrimidine-4-carboxamide (90 mg, 0.25 mmol), 1-(3-aminoazetidin-1-yl)ethanone (55 mg, 0.5 mmol) and TEA (0.1 mL) in i-PrOH (10 mL) was stirred at 80° C. for 12 h. TLC showed the reaction completed and solvent was removed under vacuum, residue was then purified by Pre-HPLC separation to give the desired product (39.6 mg, 37% yield) 1H NMR (400 MHz, METHANOL-d4) δ (ppm): 8.45 (s, 1H), 7.17 (br. s., 1H), 4.78 (br. s., 1H), 4.60 (t, J=8.4 Hz, 1H), 4.37 (t, J=9.0 Hz, 1H), 4.10 (dd, J=5.0, 9.0 Hz, 1H), 4.03 (t, J=5.8 Hz, 1H), 3.91 (dd, J=5.3, 10.3 Hz, 1H), 3.59-3.45 (m, 4H), 3.02-2.90 (m, 2H), 2.78-2.66 (m, 4H), 2.43 (s, 3H), 1.91 (s, 3H); LCMS (m/z): 430.2 (M+1).

### Example 39: (S)-6-((1-acetylpiperidin-4-yl)amino)-N-(2-hydroxy-3-(2-methyl-6,7-dihydrooxazolo[4,5-c]pyridin-5(4H)-yl)propyl)pyrimidine-4-carboxamide (Compound

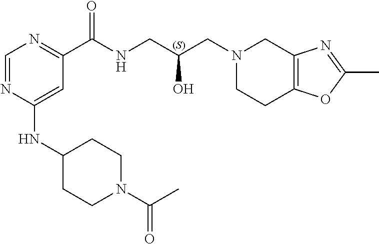

A mixture of (S)-6-chloro-N-(2-hydroxy-3-(2-methyl-6,7-dihydrooxazolo[4,5-c]pyridin-5(4H)-yl)propyl)pyrimidine-4-carboxamide (90 mg, 0.25 mmol), 1-(4-aminopiperidin-1-yl)ethanone (64 mg, 0.5 mmol) and TEA (0.1 mL) in i-PrOH (10 mL) was stirred at 80° C. for 12 h. TLC showed the reaction completed and solvent was removed under vacuum, residue was then purified by Pre-HPLC separation to give the desired product (43.4 mg, 36% yield). ¹H NMR (400 MHz, MeOD) δ (ppm): 8.40 (s, 1H), 7.11 (s, 1H), 4.45 (d, J=13.3 Hz, 1H), 4.19 (br. s., 1H), 4.07-4.00 (m, 1H), 3.95 (d, J=14.1 Hz, 1H), 3.59-3.51 (m, 3H), 3.50-3.43 (m, 1H), 3.32-3.26 (m, 1H), 2.99-2.93 (m, 2H), 2.93-2.86 (m, 1H), 2.75 (br. s., 2H), 2.71 (d, J=6.0 Hz, 2H), 2.43 (s, 3H), 2.14 (s, 3H), 2.12-1.98 (m, 2H), 1.57-1.48 (m, 1H), 1.48-1.38 (m, 1H); LCMS (m/z): 458.3 (M+1).

### Example 40: (S)-2-((1-acetylpiperidin-4-yl)amino)-N-(2-hydroxy-3-(2-methyl-6,7-dihydrooxazolo[4,5-c]pyridin-5(4H)-yl)propyl)isonicotinamide (Compound 4-4)

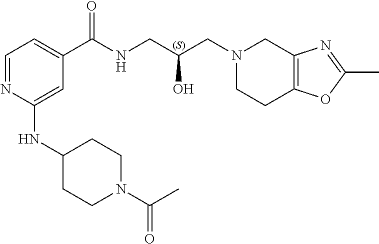

A mixture of 2-((1-acetylpiperidin-4-yl)amino)isonicotinic acid (123 mg, 0.47 mmol), (S)-1-amino-3-(2-methyl-6,7-dihydrooxazolo[4,5-c]pyridin-5(4H)-yl)propan-2-ol (100 mg, 0.47 mmol), HATU (214 mg, 0.56 mmol) and TEA (1 mL) in DCM (10 mL) was stirred at 20° C. for 12 h. TLC showed the reaction completed and solvent was removed under vacuum, residue was then purified by Pre-HPLC separation to give the desired product (16.3 mg, 7.6% yield). ¹H NMR (400 MHz, METHANOL-d4) δ (ppm): 8.03 (s, 1H), 6.89 (s, 1H), 6.82 (dd, J=1.3, 5.3 Hz, 1H), 4.44 (d, J=12.0 Hz, 1H), 4.08-4.02 (m, 1H), 4.01-3.88 (m, 2H), 3.57-3.49 (m, 3H), 3.40 (dd, J=6.8, 13.6 Hz, 1H), 3.32-3.25 (m, 1H), 3.00-2.95 (m, 2H), 2.94-2.86 (m, 1H), 2.74 (d, J=4.8 Hz, 2H), 2.72-2.62 (m, 2H), 2.43 (s, 3H), 2.14 (s, 3H), 2.11-1.99 (m, 2H), 1.54-1.45 (m, 1H), 1.44-1.35 (m, 1H); LCMS (m/z): 457.3 (M+1).

### Example 41: (S)—N-(2-hydroxy-3-(1-methyl-6,7-dihydro-1H-imidazo[4,5-c]pyridin-5(4H)-yl)propyl)-6-(oxetan-3-ylamino)pyrimidine-4-carboxamide (Compound 3-25) (S)—N-(2-hydroxy-3-(3-methyl-6,7-dihydro-3H-imidazo[4,5-c]pyridin-5(4H)-yl)propyl)-6-(oxetan-3-ylamino)pyrimidine-4-carboxamide (Compound 3-26)

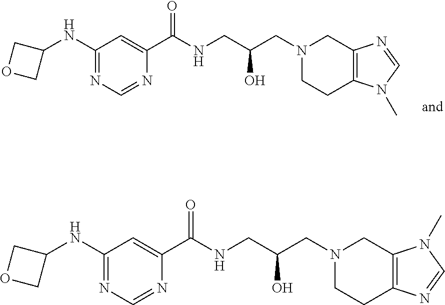 [SMILES: Cn1cnc2c1CCN(C[C@@H](O)CNC(=O)c1cc(NC3COC3)ncn1)C2 ; Cn1cnc2c1CN(C[C@@H](O)CNC(=O)c1cc(NC3COC3)ncn1)CC2]

### Step 1: 4,5,6,7-tetrahydro-1H-imidazo[4,5-c]pyridine

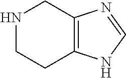

A mixture of 2-(1H-imidazol-5-yl)ethanamine hydrochloride (10.0 g, 54.3 mmol) and (CH₂O)n (2.1 g, 70.6 mmol) in H₂O (150 mL) was stirred at 100° C. for 16 h. The reaction solution was concentrated to give the desired product (7.0 g, 104.5%). LCMS (m/z): 124.2 [M+H]⁺

### Step 2: di-tert-butyl 6,7-dihydro-1H-imidazo[4,5-c]pyridine-1,5(4H)-dicarboxylate

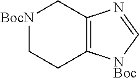 [SMILES: CC(C)(C)OC(=O)N1CCc2c(ncn2C(=O)OC(C)(C)C)C1]

To a solution of 4,5,6,7-tetrahydro-1H-imidazo[4,5-c]pyridine (6.7 g, 54.3 mmol) in THF (80 mL) and H₂O (80 mL) was added Na₂CO₃ (11.5 g, 108.6 mmol), Boc₂O (23.7 g, 108.6 mmol) was then added in portions at 0° C. The resulting solution was stirred at this temperature for 3 h, and diluted with water, extracted with EtOAc (50 mL×3). The organic layer was combined, dried and the residue was purified by column separation to afford the desired product (17.5 g, 100%). 1H NMR (CD₃OD, 400 MHz) δ (ppm): 8.09 (s, 1H), 4.55 (s, 2H), 3.58 (t, J=5.5 Hz, 2H), 2.53 (br. s., 2H), 1.62-1.53 (m, 9H), 1.42 (s, 9H). LCMS (m/z): 324.0 [M+H]⁺

### Step 3: tert-butyl 6,7-dihydro-1H-imidazo[4,5-c]pyridine-5(4H)-carboxylate

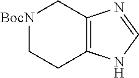 [SMILES: CC(C)(C)OC(=O)N1CCc2[nH]cnc2C1]

To a solution of di-tert-butyl 6,7-dihydro-1H-imidazo[4,5-c]pyridine-1,5(4H)-dicarboxylate (17.5 g, 54.3 mmol) in MeOH (70 mL) was added 15% aqueous of NaOH (20 mL). The mixture was stirred at 26° C. for 30 min. Solution was concentrated to remove MeOH and the residue diluted with water (60 mL) and extracted with EtOAc (50 mL×3). The organic layer was concentrated, and the residue was purified by column chromatography to give the desired product (8.0 g, 66.1%). LCMS (m/z): 224.0 [M+H]⁺

### Step 4: tert-butyl 1-methyl-6,7-dihydro-1H-imidazo[4,5-c]pyridine-5(4H)-carboxylate and tert-butyl 3-methyl-6,7-dihydro-3H-imidazo[4,5-c]pyridine-5(4H)-carboxylate

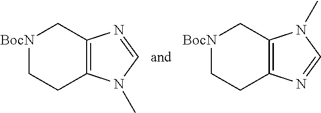 [SMILES: Cn1cnc2c1CCN(C(=O)OC(C)(C)C)C2 ; "and" ; Cn1cnc2c1CN(C(=O)OC(C)(C)C)CC2]

To a solution of tert-butyl 6,7-dihydro-1H-imidazo[4,5-c]pyridine-5(4H)-carboxylate (8.0 g, 35.9 mmol) in THF (100 mL) was added NaH (1.7 g, 42.3 mmol) at 0° C. The mixture was stirred at 26° C. for 10 min. MeI (6.0 g, 42.3 mmol) was added and the resulting mixture was stirred at 26° C. for 16 h. Reaction was then quenched with water (50 mL) and extracted with EtOAc (40 mL×3). The organic layer was concentrated to give the desired mixture (8.5 g, 100%). LCMS (m/z): 238.2 [M+H]⁺

### Step 5: 1-methyl-4,5,6,7-tetrahydro-1H-imidazo[4,5-c]pyridine and 3-methyl-4,5,6,7-tetrahydro-3H-imidazo[4,5-c]pyridine

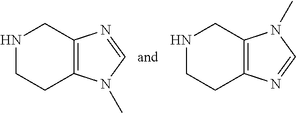

To a solution of a mixture (8.5 g, 35.9 mmol) of tert-butyl 1-methyl-6,7-dihydro-1H-imidazo[4,5-c]pyridine-5(4H)-carboxylate and tert-butyl 3-methyl-6,7-dihydro-3H-imidazo[4,5-c]pyridine-5(4H)-carboxylate in MeOH (60 mL) was added MeOH.HCl (15 mL) at 0° C. The mixture was stirred at 26° C. for 16 h. The reaction solution was then concentrated under vacuum to give the desired product (5.5 g, 112.2%). LCMS (m/z): 138.0 [M+H]⁺

### Step 6: (R)-1-methyl-5-(oxiran-2-ylmethyl)-4,5,6,7-tetrahydro-1H-imidazo[4,5-c]pyridine and (R)-3-methyl-5-(oxiran-2-ylmethyl)-4,5,6,7-tetrahydro-3H-imidazo[4,5-c]pyridine

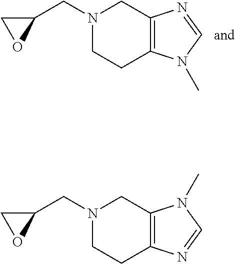

To a solution of a mixture (4.9 g, 35.9 mmol) of 1-methyl-4,5,6,7-tetrahydro-1H-imidazo[4,5-c]pyridine and 3-methyl-4,5,6,7-tetrahydro-3H-imidazo[4,5-c] pyridine in DMF (60 mL) was added KF (8.3 g, 143.6 mmol) and (S)-oxiran-2-ylmethyl 3-nitrobenzenesulfonate (11.1 g, 43.1 mmol). The resulting mixture was stirred at 26° C. for 16 h under N₂. and the reaction solution was used directly in next step without further purification. LCMS (m/z): 194.2 [M+H]⁺

### Step 7: (S)-1-amino-3-(1-methyl-6,7-dihydro-1H-imidazo[4,5-c]pyridin-5(4H)-yl)propan-2-ol and (S)-1-amino-3-(3-methyl-6,7-dihydro-3H-imidazo[4,5-c]pyridin-5(4H)-yl)propan-2-ol

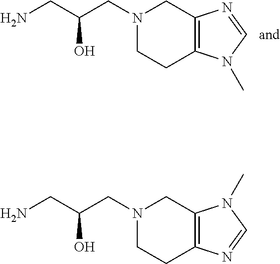

To a stirring solution of (R)-1-methyl-5-(oxiran-2-ylmethyl)-4,5,6,7-tetrahydro-1H-imidazo[4,5-c]pyridine and (R)-3-methyl-5-(oxiran-2-ylm ethyl)-4,5,6,7-tetrahydro-3H-imidazo[4,5-c]pyridine (6.9 g, 35.9 mmol) in DMF (60 mL) and EtOH (60 mL) was added aq ammonia (60 mL). The mixture was stirred at 100° C. for 4 h. TLC showed the reaction completed and the reaction solution was concentrated, residue was re-dissolved in MeOH (60 mL) and solid was filtered off. The filtrate was concentrated, and the residue was purified by column chromatography to give the desired mixture (6.0 g, 79.5%). LCMS (m/z): 211.2 [M+H]⁺

### Step 8: (S)-6-chloro-N-(2-hydroxy-3-(1-methyl-6,7-dihydro-1H-imidazo[4,5-c]pyridin-5(4H)-yl)propyl)pyrimidine-4-carboxamide and (S)-6-chloro-N-(2-hydroxy-3-(3-methyl-6,7-dihydro-3H-imidazo[4,5-c]pyridin-5(4H)-yl)propyl)pyrimidine-4-carboxamide

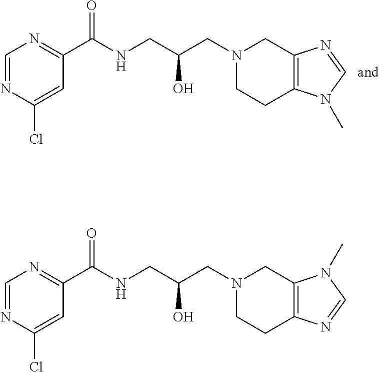 [SMILES: Cn1cnc2c1CCN(C[C@@H](O)CNC(=O)c1cc(Cl)ncn1)C2 ; "and" ; Cn1cnc2c1CN(C[C@@H](O)CNC(=O)c1cc(Cl)ncn1)CC2]

To a solution of (S)-1-amino-3-(1-methyl-6,7-dihydro-1H-imidazo[4,5-c]pyridin-5(4H)-yl)propan-2-ol and (S)-1-amino-3-(3-meth yl-6,7-dihydro-3H-imidazo[4,5-c]pyridin-5(4H)-yl)propan-2-ol (4.0 g, 19.0 mmol) in DCM (50 mL) and Et₃N (7.7 g, 76.0 mmol) was added a solution of 6-chloropyrimidine-4-carbonyl chloride (4.0 g, 22.8 mmol) in DCM (10 mL) at 0° C., and the resulting mixture was stirred at 23° C. for 2 h. Once TLC analysis showed the reaction to be complete, the reaction solution was concentrated, residue was purified by column chromatography (DCM:MeOH=10:1) to give the desired product (2.0 g, 30%). LCMS (m/z): 351.1 [M+H]⁺

### Step 9: (S)—N-(2-hydroxy-3-(1-methyl-6,7-dihydro-1H-imidazo[4,5-c]pyridin-5(4H)-yl)propyl)-6-(oxetan-3-ylamino)pyrimidine-4-carboxamide and (S)—N-(2-hydroxy-3-(3-methyl-6,7-dihydro-3H-imidazo[4,5-c]pyridin-5(4H)-yl)propyl)-6-(oxetan-3-ylamino)pyrimidine-4-carboxamide

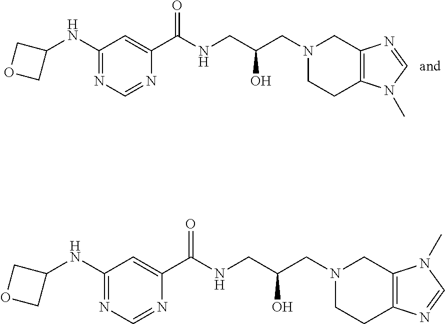 [SMILES: Cn1cnc2c1CCN(C[C@@H](O)CNC(=O)c1cc(NC3COC3)ncn1)C2 ; "and" ; Cn1cnc2c1CN(C[C@@H](O)CNC(=O)c1cc(NC3COC3)ncn1)CC2]

To a solution of (S)-6-chloro-N-(2-hydroxy-3-(1-methyl-6,7-dihydro-1H-imidazo[4,5-c]pyridin-5(4H)-yl)propyl)pyrimidine-4-carboxamide and (S)-6-chloro-N-(2-hydroxy-3-(3-methyl-6,7-dihydro-3H-imidazo[4,5-c] pyridin-5 (4H)-yl)propyl)pyrimidine-4-carboxamide (500 mg, 1.43 mmol) in i-PrOH (20 mL) and DIPEA (369 mg, 2.86 mmol) was added oxetan-3-amine (125 mg, 1.71 mmol). The resulting mixture was stirred at 80° C. for 16 h or until the reaction was shown to be complete by TLC analysis. The reaction solution was then concentrated, and the residue was purified by HPLC to give (S)-6-chloro-N-(2-hydroxy-3-(1-methyl-6,7-dihydro-1H-imidazo[4,5-c]pyridin-5 (4H)-yl)propyl)pyrimidine-4-carboxamide (85 mg, 15.4%). ¹HNMR (CD₃OD, 400 MHz) δ (ppm): 8.38 (s, 1H), 7.45 (s, 1H), 7.14 (br. s., 1H), 5.11 (br. s., 1H), 4.96 (t, J=6.8 Hz, 2H), 4.60 (t, J=6.1 Hz, 2H), 4.04 (t, J=5.8 Hz, 1H), 3.58 (s, 3H), 3.57-3.50 (m, 3H), 3.50-3.39 (m, 1H), 3.00-2.82 (m, 2H), 2.68 (d, J=6.0 Hz, 4H). LCMS (m/z): 388.2 [M+H]⁺

Also from the separation, (S)-6-chloro-N-(2-hydroxy-3-(3-methyl-6,7-dihydro-3H-imidazo[4,5-c]pyridin-5(4H)-yl)propyl)pyrimidine-4-carboxamide (39 mg, 7.0%). ¹HNMR (CH₃OD, 400 MHz) δ (ppm): 8.36 (s, 1H), 7.45 (s, 1H), 7.14 (br. s., 1H), 5.11 (br. s., 1H), 5.00-4.94 (m, 2H), 4.60 (t, J=6.3 Hz, 2H), 4.05 (t, J=5.8 Hz, 1H), 3.64 (s, 2H), 3.57 (d, J=5.5 Hz, 1H), 3.54 (s, 3H), 3.51-3.41 (m, 1H), 2.97-2.80 (m, 2H), 2.78-2.55 (m, 4H). LCMS (m/z): 388.2 [M+H]⁺

### Example 42: (S)-6-((1-acetylpiperidin-4-yl)amino)-N-(2-hydroxy-3-(1-methyl-6,7-dihydro-1H-imidazo[4,5-c]pyridin-5(4H)-yl)propyl)pyrimidine-4-carboxamide (Compound 3-35) and (S)-6-((1-acetylpiperidin-4-yl)amino)-N-(2-hydroxy-3-(3-methyl-6,7-dihydro-3H-imidazo[4,5-c]pyridin-5(4H)-yl)propyl)pyrimidine-4-carboxamide (Compound 3-30)

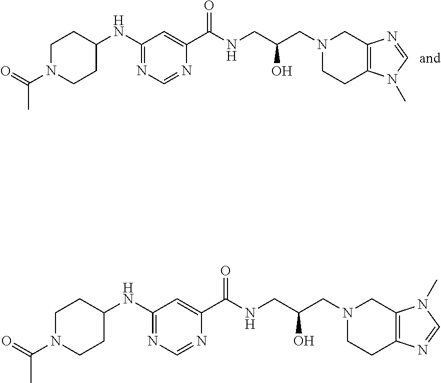 [SMILES: CC(=O)N1CCC(Nc2cc(C(=O)NC[C@H](O)CN3CCc4c(ncn4C)C3)ncn2)CC1 ; "and" ; CC(=O)N1CCC(Nc2cc(C(=O)NC[C@H](O)CN3CCc4ncn(C)c4C3)ncn2)CC1]

To a solution of (S)-6-chloro-N-(2-hydroxy-3-(1-methyl-6,7-dihydro-1H-imidazo[4,5-c]pyridin-5(4H)-yl)propyl)pyrimidine-4-carboxamide and (S)-6-chloro-N-(2-hydroxy-3-(3-methyl-6,7-dihydro-3H-imidazo[4,5-c] pyridin-5 (4H)-yl)propyl) pyrimidine-4-carboxamide (500 mg, 1.43 mmol) in i-PrOH (20 mL) was added DIPEA (369 mg, 2.86 mmol) and 1-(4-aminopiperidin-1-yl)ethanone (305 mg, 2.15 mmol). The solution was stirred at 100° C. for 16 h. Once complete by TLC analysis the reaction solution was concentrated and the residue purified by HPLC to give (S)-6-((1-acetylpiperidin-4-yl)amino)-N-(2-hydroxy-3-(1-methyl-6,7-dihydro-1H-imidazo[4,5-c]pyridin-5 (4H)-yl)propyl)pyrimidine-4-carboxamide (49 mg, 7.5%). ¹HNMR (CD₃OD, 400 MHz) δ (ppm): 8.37 (s, 1H), 7.45 (s, 1H), 7.08 (s, 1H), 4.43 (d, J=13.3 Hz, 1H), 4.17 (br. s., 1H), 4.03 (quin, J=5.9 Hz, 1H), 3.93 (d, J=13.8 Hz, 1H), 3.62-3.50 (m, 6H), 3.48-3.39 (m, 1H), 3.30-3.21 (m, 1H), 2.97-2.84 (m, 3H), 2.75-2.58 (m, 4H), 2.12 (s, 3H), 2.09-1.97 (m, 2H), 1.56-1.35 (m, 2H). LCMS (m/z): 457.3 [M+H]⁺

Also from the separation, (S)-6-((1-acetylpiperidin-4-yl)amino)-N-(2-hydroxy-3-(3-methyl-6,7-dihydro-3H-imidazo[4,5-c]pyridin-5 (4H)-yl)propyl)pyrimidine-4-carboxamide (27 mg, 4.1%). ¹HNMR (CH₃OD, 400 MHz) δ (ppm): 8.92 (s, 1H), 8.59 (s, 1H), 7.20 (br. s., 1H), 4.65 (s, 2H), 4.48 (d, J=12.5 Hz, 1H), 4.37 (br. s., 2H), 3.97 (d, J=13.8 Hz, 1H), 3.88-3.83 (m, 3H), 3.82-3.68 (m, 2H), 3.62-3.46 (m, 3H), 3.43-3.34 (m, 1H), 3.30-3.24 (m, 1H), 3.15 (d, J=5.3 Hz, 2H), 2.87 (t, J=11.5 Hz, 1H), 2.16-1.99 (m, 5H), 1.68-1.38 (m, 2H). LCMS (m/z): 457.3 [M+H]⁺

### Example 43: (S)-2-((1-acetylpiperidin-4-yl)amino)-N-(2-hydroxy-3-(1-methyl-6,7-dihydro-1H-imidazo[4,5-c]pyridin-5(4H)-yl)propyl)isonicotinamide (Compound 3-22) and (S)-2-((1-acetylpiperidin-4-yl)amino)-N-(2-hydroxy-3-(3-methyl-6,7-dihydro-3H-imidazo[4,5-c]pyridin-5(4H)-yl)propyl)isonicotinamide (Compound 3-24)

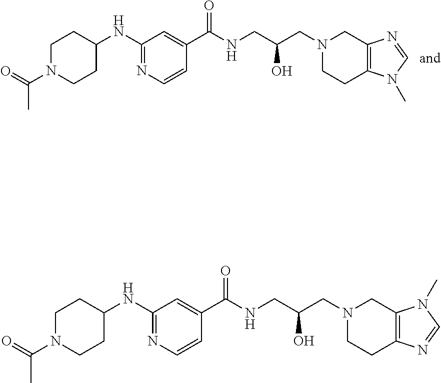 [SMILES: CC(=O)N1CCC(Nc2cc(C(=O)NC[C@H](O)CN3CCc4c(ncn4C)C3)ccn2)CC1 ; "and" ; CC(=O)N1CCC(Nc2cc(C(=O)NC[C@H](O)CN3CCc4ncn(C)c4C3)ccn2)CC1]

To a solution of 2-((1-acetylpiperidin-4-yl)amino)isonicotinic acid (500 mg, 1.9 mmol) in DCM (15 mL) was added Et₃N (385 mg, 3.8 mmol) and HATU (1.08 g, 2.9 mmol), solution then turned clear and stirred at 23° C. for 15 min, the mixture (483 mg, 2.3 mmol) of (S)-1-amino-3-(1-methyl-6,7-dihydro-1H-imidazo[4,5-c]pyridin-5(4H)-yl)propan-2-ol and (S)-1-amino-3-(3-methyl-6,7-dihydro-3H-imidazo[4,5-c]pyridin-5(4H)-yl)propan-2-ol was then added. The mixture was stirred at 23° C. for another 4 h. LCMS showed the reaction completed and the reaction solution was then concentrated and the residue purified by HPLC separation to give (S)-2-((1-acetylpiperidin-4-yl)amino)-N-(2-hydroxy-3-(1-methyl-6,7-dihydro-1H-imidazo[4,5-c]pyridin-5(4H)-yl)propyl)isonicotinamide (61 mg, 7.0%). ¹H NMR (CD₃OD, 400 MHz) δ (ppm): 8.00 (d, J=5.5 Hz, 1H), 7.48 (s, 1H), 6.88 (s, 1H), 6.81-6.75 (m, 1H), 4.44 (d, J=13.6 Hz, 1H), 4.07 (t, J=5.8 Hz, 1H), 4.02-3.88 (m, 2H), 3.67 (d, J=5.5 Hz, 1H), 3.63-3.49 (m, 6H), 3.48-3.39 (m, 1H), 3.30 (br. s., 1H), 3.03-2.84 (m, 3H), 2.78-2.61 (m, 4H), 2.17-1.99 (m, 5H), 1.56-1.33 (m, 2H). LCMS (m/z): 456.3 [M+H]⁺

Also from the separation, (S)-2-((1-acetylpiperidin-4-yl)amino)-N-(2-hydroxy-3-(3-methyl-6,7-dihydro-3H-imidazo[4,5-c]pyridin-5(4H)-yl)propyl)isonicotinamide (35 mg, 4.0%).

¹H NMR (CD₃OD, 400 MHz) δ (ppm): 8.01 (d, J=5.5 Hz, 1H), 7.47 (s, 1H), 6.88 (s, 1H), 6.79 (d, J=5.5 Hz, 1H), 4.44 (d, J=13.1 Hz, 1H), 4.08 (quin, J=5.9 Hz, 1H), 4.03-3.89 (m, 2H), 3.66 (s, 2H), 3.61-3.49 (m, 4H), 3.47-3.39 (m, 1H), 3.32-3.23 (m, 1H), 2.98-2.86 (m, 3H), 2.82-2.62 (m, 4H), 2.18-1.98 (m, 5H), 1.54-1.34 (m, 2H). LCMS (m/z): 456.3 [M+H]⁺

### Example 44: (S)-2-((1-acetylpiperidin-4-yl)amino)-N-(2-hydroxy-3-(1-methyl-4,5-dihydro-1H-pyrazolo[3,4-c]pyridin-6(7H)-yl)propyl)isonicotinamide (Compound 3-37)

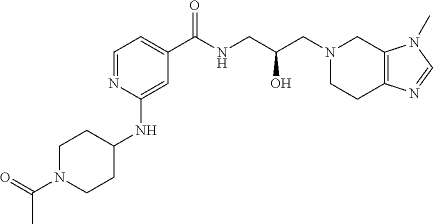 [SMILES: CC(=O)N1CCC(Nc2cc(C(=O)NC[C@H](O)CN3CCc4ncn(C)c4C3)ccn2)CC1]

### Step 1: (E)-1-benzyl-4-((dimethylamino)methylene)piperidin-3-one

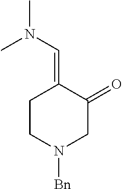 [SMILES: CN(C)/C=C1\CCN(Cc2ccccc2)CC1=O]

To a solution of 1-benzylpiperidin-3-one (5.0 g×3, 26.42 mmol×3) in DMF (40 mL×3) was added DMF-DMA (17.6 g×3, 132.10 mmol×3). The reaction mixture was stirred and heated at 70° C. for 16 h, at which time TLC showed the reaction was finished. The reaction mixture was quenched with water (600 mL) and extracted with EtOAc (250 mL×6). The organic layers were dried over Na₂SO₄, concentrated and purified by silica gel column chromatography (DCM:MeOH=100:1˜20:1) to give product (E)-1-benzyl-4-((dimethylamino)methylene)piperidin-3-one (7.38 g, yield: 38.2%) as a yellow oil. LCMS (m/z): 245.2 [M+H]⁺

### Step 2: 6-benzyl-4,5,6,7-tetrahydro-1H-pyrazolo[3,4-c]pyridine

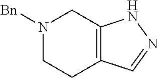 [SMILES: c1ccc(CN2CCc3cn[nH]c3C2)cc1]

To a solution of (E)-1-benzyl-4-((dimethylamino)methylene)piperidin-3-one (7.38 g, 30.246 mmol) in EtOH (80 mL) was added N₂H₄.H₂O (3.02 g, 60.492 mmol). After addition, the mixture was stirred and heated at 90° C. for 1 h, at which time TLC showed the completion of the reaction. The mixture was concentrated and purified by flash chromatography (DCM:MeOH=100:1˜20:1) to give the product 6-benzyl-4,5,6,7-tetrahydro-2H-pyrazolo[3,4-c]pyridine (5.3 g, yield: 82.3%) as a light yellow solid. 1H NMR (400 MHz, CDCl₃) δ (ppm): 10.53 (br. s., 1H), 7.43-7.38 (m, 2H), 7.38-7.33 (m, 2H), 7.31-7.28 (m, 2H), 3.75 (s, 2H), 3.64 (s, 2H), 2.79-2.73 (m, 2H), 2.71-2.65 (m, 2H).

### Step 3: 4,5,6,7-tetrahydro-1H-pyrazolo[3,4-c]pyridine

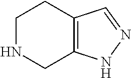

To a solution of 6-benzyl-4,5,6,7-tetrahydro-2H-pyrazolo[3,4-c]pyridine (5.3 g, 24.850 mmol) in MeOH (50 mL) was added wet 10% Pd(OH)₂/C (1.0 g). The reaction mixture was stirred at 20° C. under H₂ (50 Psi) for 48 h, at which time TLC showed the completion of the reaction. The mixture was filtered and concentrated to give the compound 4,5,6,7-tetrahydro-1H-pyrazolo [3,4-c]pyridine (2.99 g, yield: 97.8%) as a white solid. This crude was used in next step without further purification.

### Step 4: tert-butyl 4,5-dihydro-1H-pyrazol[3,4-c]pyridine-6(7H)-carboxylate

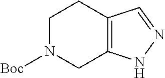 [SMILES: CC(C)(C)OC(=O)N1CCc2cn[nH]c2C1]

To the solution of 4,5,6,7-tetrahydro-1H-pyrazolo[3,4-c]pyridine (2.99 g, 24.277 mmol) in DCM (50 mL) was added TEA (7.37 g, 72.831 mmol) and Boc₂O (5.29 g, 24.277 mmol) at 0° C. After addition, the mixture was stirred at 15° C. for 2 h, at which time TLC showed the completion of the reaction. The reaction mixture was diluted with DCM and washed with brine, solvent was removed and residue was purified by silica gel column chromatography (DCM:MeOH=100:1˜30:1) to give the product tert-butyl 4,5-dihydro-1H-pyrazolo[3,4-c]pyridine-6(7H)-carboxylate (4.3 g, yield: 79.6%) as a yellow oil. 1H NMR (400 MHz, METHANOL-d4) δ (ppm): 7.40 (s, 1H), 4.54 (s, 2H), 3.64 (t, J=5.6 Hz, 2H), 2.62 (t, J=5.6 Hz, 2H), 1.48 (s, 9H).

### Step 5: tert-butyl 1-methyl-4,5-dihydro-1H-pyrazolo[3,4-c]pyridine-6(7H)-carboxylate

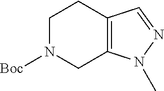 [SMILES: Cn1ncc2c1CN(C(=O)OC(C)(C)C)CC2]

The mixture of tert-butyl 4,5-dihydro-1H-pyrazolo[3,4-c]pyridine-6(7H)-carboxylate (4.0 g, 17.937 mmol) in dry DMF (100 mL) was added NaH (861 mg, 35.874 mmol) at 0° C., after stirring at this temperature for 10 min, MeI (3.82 g, 26.906 mmol) was added and the resulting mixture was stirred at 15° C. for 16 h, at which time TLC showed the completion of the reaction. The reaction mixture was quenched with aq.NH₄Cl (200 mL) at 0° C., diluted with water and was extracted with DCM (100 mL×3). The organic layers were dried over Na₂SO₄, concentrated and purified by silica gel column chromatography (PE:EA=100:1˜1:1) to give tert-butyl 1-methyl-4,5-dihydro-1H-pyrazolo[3,4-c]pyridine-6(7H)-carboxylate (4.1 g, yield: 97.4%) as a light yellow oil. LCMS (m/z): 182.1 [M+H]⁺

### Step 6: 1-methyl-4,5,6,7-tetrahydro-1H-pyrazolo[3,4-c]pyridine

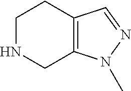

To a solution of tert-butyl 1-methyl-4,5-dihydro-1H-pyrazolo[3,4-c]pyridine-6(7H)-carboxylate (4.1 g, 17.30 mmol) in MeOH (100 mL) was added 4M HCl.EtOAc (20 mL) at 0° C. The reaction mixture was stirred at 15° C. for 16 h, at which time TLC showed the completion of the reaction. The mixture was concentrated to give a the title compound 1-methyl-4,5,6,7-tetrahydro-1H-pyrazolo[3,4-c]pyridine (3.5 g, HCl salt) as a white solid. This crude mixture was used in next step without further purification.

### Step 7: (R)-1-methyl-6-(oxiran-2-ylmethyl)-4,5,6,7-tetrahydro-1H-pyrazolo[3,4-c]pyridine

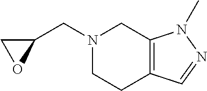

To a solution of 1-methyl-4,5,6,7-tetrahydro-1H-pyrazolo[3,4-c]pyridine (3200 mg, 23.327 mmol) and KF (5420 mg, 93.308 mmol) in THF (350 mL) was added (S)-oxiran-2-ylmethyl 3-nitrobenzenesulfonate (6050 mg, 23.327 mmol) and K₂CO₃ (6450 mg, 46.654 mmol). The reaction mixture was stirred at 25° C. for 24 h. Then the mixture was filtered and washed with EtOAc. The organic layer was concentrated to give compound (R)-1-methyl-6-(oxiran-2-ylmethyl)-4,5,6,7-tetrahydro-1H-pyrazolo[3,4-c]pyridine (4.51 g, crude) as a yellow oil. This crude was used in next step without further purification.

### Step 8: (S)-1-amino-3-(1-methyl-4,5-dihydro-1H-pyrazolo[3,4-c]pyridin-6(7H)-yl)propan-2-ol

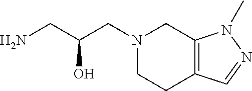

To a solution of compound (R)-1-methyl-6-(oxiran-2-ylmethyl)-4,5,6,7-tetrahydro-1H-pyrazolo [3,4-c]pyridine (4.51 g, 23.338 mmol) in DMF (50 mL) was added NH₃.H₂O (500 mL). The reaction mixture was stirred at 50° C. for 16 h. The reaction was concentrated to give product (S)-1-amino-3-(1-methyl-4,5-dihydro-1H-pyrazolo[3,4-c]pyridin-6(7H)-yl)propan-2-ol (4.91 g, crude) as a yellow oil. This crude was used in next step without further purification.

### Step 9: (S)-tert-butyl (2-hydroxy-3-(1-methyl-4,5-dihydro-1H-pyrazolo[3,4-c]pyridin-6(7H)-yl)propyl)carbamate

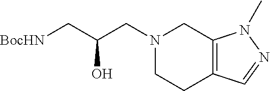 [SMILES: Cn1ncc2c1CN(C[C@@H](O)CNC(=O)OC(C)(C)C)CC2]

The mixture of (S)-1-amino-3-(1-methyl-4,5-dihydro-1H-pyrazolo[3,4-c]pyridin-6(7H)-yl) propan-2-ol (4.91 g crude, 23.381 mmol) in water (200 mL) was added K₂CO₃ (6.45 g, 46.762 mmol) and Boc₂O (3.77 g, 17.294 mmol). After addition, the mixture was stirred at 15° C. for 18 h, at which time TLC showed the completion of the reaction. The reaction mixture was extracted with EtOAc (250 mL×2) and the organic layers were concentrated and purified by silica gel column chromatography (DCM:MeOH=100:1˜20:1) to give the product (S)-tert-butyl (2-hydroxy-3-(1-methyl-4,5-dihydro-1H-pyrazolo[3,4-c]pyridin-6(7H)-yl)propyl)carbamate (2.71 g, yield: 50.4%) as a yellow oil. LCMS (m/z): 311.2 [M+H]⁺

### Step 10: (S)-1-amino-3-(1-methyl-4,5-dihydro-1H-pyrazolo[3,4-c]pyridin-6(7H)-yl)propan-2-ol

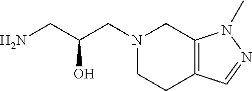

To a solution of (S)-tert-butyl (2-hydroxy-3-(1-methyl-4,5-dihydro-1H-pyrazolo[3,4-c]pyridin-6(7H)-yl)propyl)carbamate (2.71 g, 8.742 mmol) in MeOH (130 mL) was added 4M HCl.EtOAc (15 mL) at 0° C. The reaction mixture was stirred at 15° C. for 16 h, solid was precipitated and collected by filtration to afford desired product (2.7 g, HCl salt) as a white solid. LCMS (m/z): 211.2 [M+H]⁺

### Step 11: (S)-2-((1-acetylpiperidin-4-yl)amino)-N-(2-hydroxy-3-(1-methyl-4,5-dihydro-1H-pyrazolo[3,4-c]pyridin-6(7H)-yl)propyl)isonicotinamide

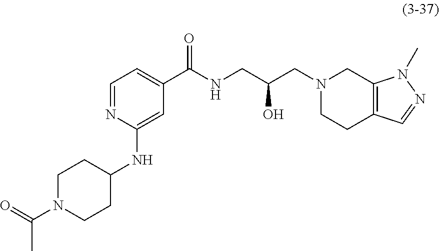 [SMILES: CC(=O)N1CCC(Nc2cc(C(=O)NC[C@H](O)CN3CCc4cnn(C)c4C3)ccn2)CC1]

To the stirring solution of compound 2-((1-acetylpiperidin-4-yl)amino)isonicotinic acid (150 mg, 0.570 mmol) in DCM (10 mL) was added TEA (288 mg, 2.849 mmol) and HATU (325 mg, 0.855 mmol), the mixture was stirred at 15° C. for 20 min, a solution of (S)-1-amino-3-(1-methyl-4,5-dihydro-1H-pyrazolo[3,4-c]pyridin-6(7H)-yl)propan-2-ol (360 mg, 1.170 mmol, in 5 mL DCM) was then added and the resulting solution was stirred at 15° C. for another 16 h, at which time TLC showed the completion of the reaction. Then the reaction mixture was concentrated and the residue purified by prep-HPLC and prep-SFC to give the title compound (S)-2-((1-acetylpiperidin-4-yl)amino)-N-(2-hydroxy-3-(1-methyl-4,5-dihydro-1H-pyrazolo[3,4-c]pyridin-6(7H)-yl)propyl)isonicotinamide (8.6 mg, yield: 3.3%) as a white solid. 1H NMR (400 MHz, METHANOL-d4) δ (ppm): 8.00 (d, J=5.6 Hz, 1H), 7.23 (s, 1H), 6.86 (s, 1H), 6.78 (dd, J=1.6, 5.6 Hz, 1H), 4.43 (d, J=12.0 Hz, 1H), 4.12-4.03 (m, 1H), 4.00-3.89 (m, 2H), 3.73-3.66 (m, 5H), 3.57-3.51 (m, 1H), 3.45-3.37 (m, 1H), 3.30-3.23 (m, 1H), 2.94-2.80 (m, 3H), 2.76-2.66 (m, 2H), 2.65-2.58 (m, 2H), 2.14-1.99 (m, 5H), 1.51-1.34 (m, 2H). LCMS (m/z): 456.3 [M+H]⁺

### Example 45: (S)-2-((1-acetylpiperidin-4-yl)amino)-N-(3-(5,6-dihydro-1,7-naphthyridin-7(8H)-yl)-2-hydroxypropyl)isonicotinamide (Compound 2-21)

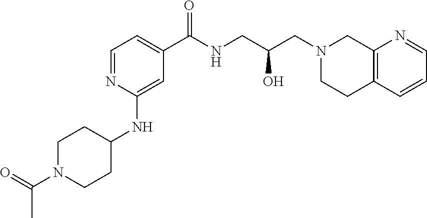 [SMILES: CC(=O)N1CCC(Nc2cc(C(=O)NC[C@H](O)CN3CCc4cccnc4C3)ccn2)CC1]

### Step 1: methyl 3-methylpicolinate

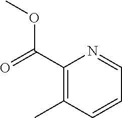

A mixture of 2-bromo-3-methylpyridine (5.0 g, 29.0 mmol), Pd(dppf)Cl₂ (2.1 g, 2.9 mmol) and Et₃N (8.8 g, 87 mmol) in MeOH (250 mL) was stirred at 80° C. and 50 Psi under CO atmosphere for 16 h. The mixture was filtered and the filtrate was concentrated. The residue was purified by column chromatography (PE:EA=5:1) to give the desired product (4.1 g, 93.6%). LCMS (m/z): 152.0 [M+H]⁺

### Step 2: methyl 3-(bromomethyl)picolinate

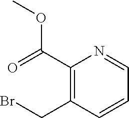

A mixture of methyl 3-methylpicolinate (4.1 g, 27.1 mmol), NBS (5.8 g, 32.5 mmol) (5.8 g, 32.5 mmol), AIBN (100 mg, 0.61 mmol) in CCl₄ (55 mL) was stirred at 90° C. for 16 h under N₂. The mixture was filtered and the filtrate was concentrated in vacuum to give the crude product. The crude product was purified by column chromatography (PE:EA=5:1) to give the desired product (5.0 g, 80.6%). 1H NMR (400 MHz, CDCl₃) δ (ppm): 8.67 (dd, J=1.6, 4.6 Hz, 1H), 7.91 (dd, J=1.5, 7.9 Hz, 1H), 7.48 (dd, J=4.6, 7.9 Hz, 1H), 4.95 (s, 2H), 4.07-4.03 (m, 3H). LCMS (m/z): 229.9 [M+H]⁺

### Step 3: methyl 3-(cyanomethyl)picolinate

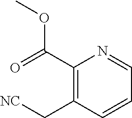

To a solution of methyl 3-(bromomethyl)picolinate (6.0 g, 26.0 mmol) in CH₃CN (200 mL) was added TBAF (10.2 g, 39.0 mmol) and TMSCN (5.2 g, 52.0 mmol) at 0° C. The mixture was stirred at 30° C. for 16 h under N₂. The solution was then diluted with DCM and washed with sat. NaCl. The organic layer was dried over Na₂SO₄ and concentrated in vacuum to give the crude product. This crude product was purified by column chromatography (PE:EA=5:1-2:1) to afford the desired product (2.3 g, 50.3%). ¹H NMR (400 MHz, CDCl₃) δ (ppm): 8.76 (dd, J=1.5, 4.6 Hz, 1H), 8.04 (td, J=0.8, 8.0 Hz, 1H), 7.58 (dd, J=4.6, 8.0 Hz, 1H), 4.31 (s, 2H), 4.04 (s, 3H). LCMS (m/z): 177.0 [M+H]⁺

### Step 4: 6, 7-dihydro-1,7-naphthyridin-8(5H)-one

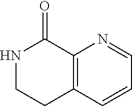 [SMILES: O=C1NCCc2cccnc21]

A mixture of methyl 2-(cyanomethyl)nicotinate (2.3 g, 13.0 mmol) and Raney Ni (400 mg) in EtOH (40 mL) and H₂O (40 mL) was hydrogenated at 50° C. under H₂ and 50 Psi for 16 h. The mixture was filtered, and the filtrate was concentrated to give the desired product (2.1 g, 109.4%). 1H NMR (400 MHz, CD₃OD) δ (ppm): 8.60 (d, J=3.4 Hz, 1H), 7.89-7.75 (m, 1H), 7.53 (dd, J=4.8, 7.8 Hz, 1H), 3.65-3.49 (m, 2H), 3.10 (t, J=6.7 Hz, 2H). LCMS (m/z): 149.0 [M+H]⁺

### Step 5: 5,6,7,8-tetrahydro-1,7-naphthyridine

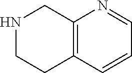

To a solution of 6,7-dihydro-1,7-naphthyridin-8(5H)-one (2.1 g, 14.2 mmol) in a mixed solution of THF (300 mL) and DCM (100 mL) was added dropwise BH₃.Me₂S (14.2 mL, 142 mmol, 10 M) at 0° C. The mixture was stirred at 80° C. for 16 h. Reaction was quenched with MeOH at −78° C. and stirred at 30° C. for 30 min. HCl/MeOH (20 mL) was added and the mixture was stirred at 30° C. for another 16 h. The resulting solution was concentrated in vacuum to give the desired product (2.0 g, crude). LCMS (m/z): 135.1 [M+H]⁺

### Step 5: (R)-7-(oxiran-2-ylmethyl)-5,6,7,8-tetrahydro-1,7-naphthyridine

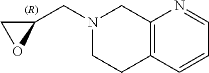

To a solution of 5,6,7,8-tetrahydro-1,7-naphthyridine (2.0 g, 14.9 mmol) in DMF (20 mL) was added Et₃N (1.5 g, 14.9 mmol), KF (3.4 g, 59.6 mmol) and (S)-oxiran-2-ylmethyl 3-nitrobenzenesulfonate (4.6 g, 17.9 mmol). The mixture was stirred at 30° C. for 16 h. The reaction mixture was filtered and the filtrate was used in next step. LCMS (m/z): 191.1 [M+H]⁺

### Step 6: (S)-1-amino-3-(5,6-dihydro-1,7-naphthyridin-7(8H)-yl)propan-2-ol

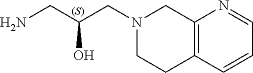

To a solution of (R)-7-(oxiran-2-ylmethyl)-5,6,7,8-tetrahydro-1,7-naphthyridine (2.7 g, 14.2 mmol) in DMF (20 mL) and EtOH (40 mL) was added NH₃H₂O (100 mL). The mixture was stirred at 70° C. for 3 h. After that, the reaction solution was concentrated, and the residue was re-dissolved in MeOH (30 mL) and filtered. The filtrate was concentrated to give the desired product (3.0 g, crude). The crude product was used in next step without further purification. LCMS (m/z): 208.2 [M+H]⁺

### Step 7: (S)-2-((1-acetylpiperidin-4-yl)amino)-N-(3-(5,6-dihydro-1,7-naphthyridin-7(8H)-yl)-2-hydroxypropyl)isonicotinamide

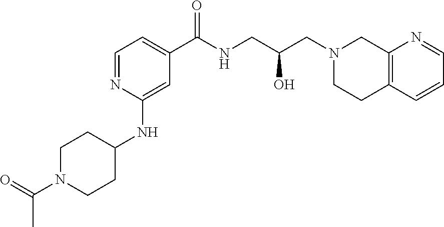 [SMILES: CC(=O)N1CCC(Nc2cc(C(=O)NC[C@H](O)CN3CCc4cccnc4C3)ccn2)CC1]

To a solution of 2-((1-acetylpiperidin-4-yl)amino)isonicotinic acid (300 mg, 1.14 mmol) in DCM (10 mL) and DIPEA (0.61 mL, 3.42 mmol) was added Bop-Cl (318 mg, 1.25 mmol) at 25° C. The resulting solution was stirred at 25° C. for 30 mins and (S)-1-amino-3-(5,6-dihydro-1,7-naphthyridin-7(8H)-yl)propan-2-ol (235 mg, 1.14 mmol) was added and stirred at 25° C. for another 16 h, and concentrated in vacuum to give the crude product. The crude product was purified by prep-HPLC to give the desired product (100 mg, 19%). ¹H NMR (400 MHz, MeOD) δ (ppm): 8.30 (d, J=4.8 Hz, 1H), 7.99 (d, J=5.5 Hz, 1H), 7.62 (d, J=7.8 Hz, 1H), 7.24 (dd, J=4.9, 7.7 Hz, 1H), 6.92-6.85 (m, 1H), 6.80 (dd, J=1.6, 5.5 Hz, 1H), 4.44 (d, J=11.8 Hz, 1H), 4.16-4.06 (m, 1H), 4.01-3.92 (m, 2H), 3.81 (s, 2H), 3.57 (dd, J=5.1, 13.6 Hz, 1H), 3.44 (dd, J=6.7, 13.6 Hz, 1H), 3.32-3.26 (m, 1H), 2.97-2.89 (m, 5H), 2.73-2.67 (m, 2H), 2.15-2.02 (m, 5H), 1.51-1.36 (m, 2H). LCMS (m/z): 453.3 [M+H]⁺

### Example 46: (S)-2-((1-acetylpiperidin-4-yl)amino)-N-(3-(3,4-dihydro-2,7-naphthyridin-2(1H)-yl)-2-hydroxypropyl)isonicotinamide (Compound 2-19)

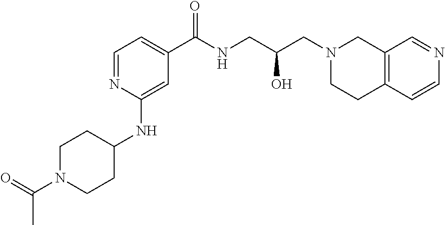 [SMILES: CC(=O)N1CCC(Nc2cc(C(=O)NC[C@H](O)CN3CCc4ccncc4C3)ccn2)CC1]

### Step 1: 4-methylnicotinonitrile

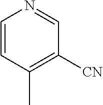

A mixture of 3-bromo-4-methylpyridine (9 g, 0.052 mol), Zn(CN)₂ (3.7 g, 0.031 mol), Pd₂(dba)₃ (2.4 g, 2.6 mmol), dppf (2.9 g, 5.2 mmol) and Zn (0.34 g, 0.052 mol) in DMF (100 mL) was stirred at 100° C. under N₂ atmosphere for 16 h. The mixture was filtered and the filtrate was concentrated in vacuum to give the crude product. The crude product was purified by column to give the desired product (5 g, yield: 82%). LCMS (m/z): 119.1 [M+H]+.

### Step 2: ethyl 2-(3-cyanopyridin-4-yl)acetate

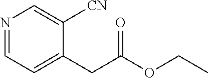

To a solution of 4-methylnicotinonitrile (2.3 g, 19.5 mmol) and Et₂CO₃ (23 g, 195 mmol) in THF (50 mL) was added NaH (3.8 g, 97.5 mmol) at 0° C. The resulting mixture was stirred under 60° C. for 16 h. Then the reaction was quenched with aq.NH₄Cl (50 mL) at 0° C. and extracted with EA (100 mL×2). The combined organic layers was dried over Na₂SO₄ and concentrated in vacuum to give the crude product. The crude product was purified by column to give the desired product (1.25 g, yield: 34%). 1H NMR (400 MHz, CDCl₃) δ (ppm): 8.89 (s, 1H), 8.77 (d, J=5.1 Hz, 1H), 7.43 (d, J=5.1 Hz, 1H), 4.24 (q, J=7.2 Hz, 2H), 3.90 (s, 2H), 1.34-1.28 (m, 3H). LCMS (m/z): 191.1 [M+H]⁺.

### Step 3: 1,2-dihydro-2,7-naphthyridin-3(4H)-one

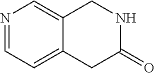 [SMILES: O=C1Cc2ccncc2CN1]

A mixture of ethyl 2-(3-cyanopyridin-4-yl)acetate (1.25 g, 6.6 mmol) and Raney Ni (1.2 g) in a mixed solution of EtOH (20 mL) and H₂O (20 mL) was hydrogenated at 50° C. under H₂ (50 Psi) for 16 h. The mixture was filtered, and the filtrate was concentrated to give the desired product (750 mg, 77%). 1H NMR (400 MHz, CDCl₃) δ (ppm): 8.58-8.45 (m, 2H), 7.16 (d, J=5.0 Hz, 1H), 6.25 (br. s., 1H), 4.60 (s, 2H), 3.63 (s, 2H). LCMS (m/z): 149.0 [M+H]⁺

### Step 4: 1,2,3,4-tetrahydro-2,7-naphthyridine

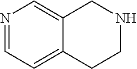

To a solution of 1,2-dihydro-2,7-naphthyridin-3(4H)-one (750 mg, 5.07 mmol) in THF (300 mL) and DCM (100 mL) was added dropwise BH₃.Me₂S (5.07 mL, 50.7 mmol, 10 M) at 0° C. The mixture was stirred at 80° C. for 16 h, quenched with MeOH at −78° C. and stirred at 30° C. for 30 min. HCl/MeOH (20 mL) was added and the mixture was stirred at 30° C. for another 16 h. The resulting mixture was concentrated in vacuum to give the desired product (400 mg, crude). LCMS (m/z): 135.1 [M+H]⁺

### Step 5: (R)-2-(oxiran-2-ylmethyl)-1,2,3,4-tetrahydro-2,7-naphthyridine

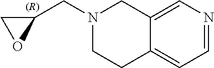

To the solution of 1,2,3,4-tetrahydro-2,7-naphthyridine (582 mg, 4.35 mmol) in DMF (20 mL) was added Et₃N (605 mg, 4.35 mmol), KF (1 g, 17.4 mmol) and (S)-oxiran-2-ylmethyl 3-nitrobenzenesulfonate (1.3 g, 4.35 mmol). The mixture was stirred at 30° C. for 16 h. The reaction mixture was filtered and the filtrate was used directly in next step without purification. LCMS (m/z): 191.1 [M+H]⁺

### Step 6: (S)-1-amino-3-(3,4-dihydro-2,7-naphthyridin-2(1H)-yl)propan-2-ol

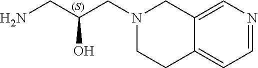

To a solution of (R)-2-(oxiran-2-ylmethyl)-1,2,3,4-tetrahydro-2,7-naphthyridine (826 mg, 4.35 mmol) in DMF (20 mL) and EtOH (20 mL) was added NH₃.H₂O (40 mL). The mixture was stirred at 70° C. for 3 h. LCMS showed the reaction completed and the solution was concentrated. The residue was re-dissolved in MeOH (30 mL) and filtered. The filtrate was concentrated to give the desired product (600 mg, 66.7%). The crude product was used in next step without further purification. LCMS (m/z): 208.2 [M+H]⁺.

### Step 7: (S)-2-((1-acetylpiperidin-4-yl)amino)-N-(3-(3,4-dihydro-2,7-naphthyridin-2(1H)-yl)-2-hydroxypropyl)isonicotinamide

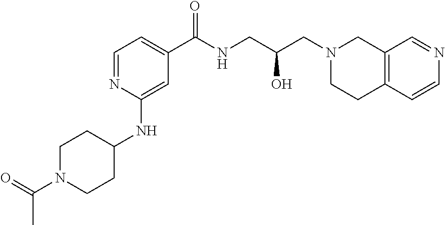 [SMILES: CC(=O)N1CCC(Nc2cc(C(=O)NC[C@H](O)CN3CCc4ccncc4C3)ccn2)CC1]

To a solution of 2-((1-acetylpiperidin-4-yl)amino)isonicotinic acid (300 mg, 1.14 mmol) in DCM (10 mL) and DIPEA (0.61 mL, 3.42 mmol) was added Bop-Cl (318 mg, 1.25 mmol) at 25° C. The mixture was stirred at 25° C. for 30 mins and (S)-1-amino-3-(3,4-dihydro-2,7-naphthyridin-2(1H)-yl)propan-2-ol (235 mg, 1.14 mmol) was added. The resulting mixture was stirred at 25° C. for another 16 h. The solvent was then removed and the residue purified by prep-HPLC to give the desired product (100 mg, 19%). 1H NMR (400 MHz, METHANOL-d₄) δ (ppm): 8.34-8.19 (m, 2H), 7.99 (d, J=5.4 Hz, 1H), 7.21 (d, J=5.1 Hz, 1H), 6.89 (s, 1H), 6.81 (dd, J=1.4, 5.4 Hz, 1H), 4.44 (d, J=13.6 Hz, 1H), 4.12 (quin, J=6.0 Hz, 1H), 4.02-3.91 (m, 2H), 3.81 (s, 2H), 3.57 (dd, J=5.0, 13.6 Hz, 1H), 3.43 (dd, J=6.7, 13.6 Hz, 1H), 3.32-3.25 (m, 1H), 3.01-2.86 (m, 5H), 2.75-2.64 (m, 2H), 2.14 (s, 4H), 2.11-2.01 (m, 2H), 1.52-1.34 (m, 2H). LCMS (m/z): 453.3 [M+H]⁺

### Example 47: (S)-2-((1-acetylpiperidin-4-yl)amino)-N-(3-(7,8-dihydro-1,6-naphthyridin-6(5H)-yl)-2-hydroxypropyl)isonicotinamide (Compound 2-20)

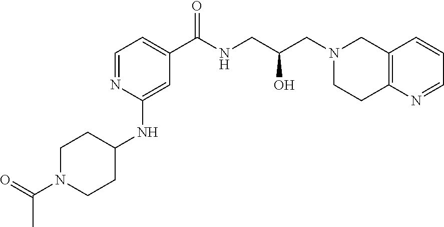 [SMILES: CC(=O)N1CCC(Nc2cc(C(=O)NC[C@H](O)CN3CCc4ncccc4C3)ccn2)CC1]

### Step 1: Methyl 2-methylnicotinate

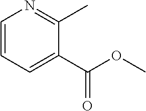

A mixture of 3-bromo-2-methylpyridine (5.0 g, 29.0 mmol), Pd(dppf)Cl₂ (2.1 g, 2.9 mmol) and Et₃N (8.8 g, 87 mmol) in MeOH (250 mL) was stirred at 80° C. and 50 Psi under CO for 16 h. Solid was then filtered out and the filtrate was concentrated. The residue was purified by column chromatography (PE:EA=5:1) to give the desired product (4.1 g, 93.6%). ¹H NMR (CD₃OD, 400 MHz) δ (ppm): 8.57-8.42 (m, 1H), 8.08 (d, J=8.0 Hz, 1H), 7.10 (dd, J=4.8, 7.8 Hz, 1H), 3.81 (s, 3H), 2.73 (s, 3H). LCMS (m/z): 152.0 [M+H]⁺

### Step 2: methyl 2-(bromomethyl)nicotinate

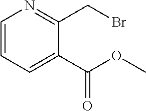

A mixture of methyl 2-methylnicotinate (4.1 g, 27.1 mmol), NBS (5.8 g, 32.5 mmol) (5.8 g, 32.5 mmol) AIBN (100 mg, 0.61 mmol) in CCl₄ (55 mL) was stirred at 90° C. for 16 h under N₂. The solution was diluted with water (25 mL) and extracted with DCM (15 mL×3). The organic layer was combined and concentrated, and the residue was purified by column chromatography (PE:EA=5:1) to give the desired product (5.0 g, 80.6%). LCMS (m/z): 229.9 [M+H]⁺

### Step 3: Methyl 2-(cyanomethyl)nicotinate

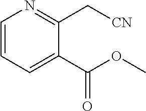

To a solution of methyl 2-(bromomethyl)nicotinate (6.0 g, 26.0 mmol) in CH₃CN (200 mL) was added TBAF (10.2 g, 39.0 mmol) and TMSCN (5.2 g, 52.0 mmol) at 0° C. The mixture was then stirred at 30° C. for 16 h under N₂. The solution of the reaction was washed with water (30 mL) and extracted with EtOAc (15 mL×3). The organic layer was concentrated, and the residue was purified by column chromatography (PE:EA=5:1-2:1) to give the desired product (2.3 g, 50.3%). LCMS (m/z): 177.0 [M+H]⁺

### Step 4: 7,8-dihydro-1,6-naphthyridin-5(6H)-one

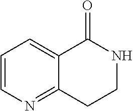 [SMILES: O=C1NCCc2ncccc21]

Methyl 2-(cyanomethyl)nicotinate (2.3 g, 13.0 mmol) and Raney Ni (400 mg) in a mixed solution of MeOH (40 mL) and H₂O (40 mL) was hydrogenated at 50° C. under H₂ (50 Psi) for 16 h. Solid was filtered out, and the filtrate was concentrated to give the desired product (2.1 g crude, 109.4%). LCMS (m/z): 149.0 [M+H]⁺

### Step 5: 5,6,7,8-tetrahydro-1,6-naphthyridine

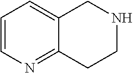

To a solution of 7,8-dihydro-1,6-naphthyridin-5(6H)-one (2.1 g, 14.2 mmol) in a mixture solution of THF (300 mL) and DCM (100 mL) was added dropwise BH₃.Me₂S (14.2 mL, 142 mmol, 10M) at 0° C. The solution was stirred at 90° C. for 16 h. The reaction solution was cooled to 30° C. and MeOH (25 mL) was added slowly, and then stirred at 30° C. for 15 min. HCl in 1,4-dioxane (4M, 20 mL) was added dropwise at 0° C., the mixture was stirred at 90° C. for 3 h. The reaction solution was cooled to 30° C. again and concentrated to give the desired product (2.0 g crude, 105.3%). LCMS (m/z): 135.1 [M+H]⁺

### Step 6: (R)-6-(oxiran-2-ylmethyl)-5,6,7,8-tetrahydro-1,6-naphthyridine

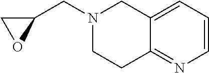 [SMILES: c1cnc2c(c1)CN(C[C@@H]1CO1)CC2]

To a solution of 5,6,7,8-tetrahydro-1,6-naphthyridine (2.0 g, 14.9 mmol) in DMF (20 mL) was added Et₃N (1.5 g, 14.9 mmol), KF (3.4 g, 59.6 mmol) and (S)-oxiran-2-ylmethyl 3-nitrobenzenesulfonate (4.6 g, 17.9 mmol). The mixture was stirred at 30° C. under N₂ for 16 h. Once the reaction was deemed complete, the reaction solution was used directly to the next step. LCMS (m/z): 191.1 [M+H]⁺

### Step 7: (S)-1-amino-3-(7,8-dihydro-1,6-naphthyridin-6(5H)-yl)propan-2-ol

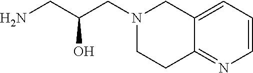 [SMILES: NC[C@H](O)CN1CCc2ncccc2C1]

To a solution of (R)-6-(oxiran-2-ylmethyl)-5,6,7,8-tetrahydro-1,6-naphthyridine in DMF (20 mL) and EtOH (20 mL) was added NH₃.H₂O (50 mL). The mixture was stirred at 100° C. for 3 h. The reaction solution was concentrated and the residue was dissolved in MeOH (30 mL) and filtered. The filtrate was concentrated, and the residue purified by column chromatography (DCM:MeOH=10:1) to give the desired product (500 mg, 16.2% two steps). LCMS (m/z): 208.2 [M+H]⁺

### Step 8: (S)-2-((1-acetylpiperidin-4-yl)amino)-N-(3-(7,8-dihydro-1,6-naphthyridin-6(5H)-yl)-2-hydroxypropyl)isonicotinamide

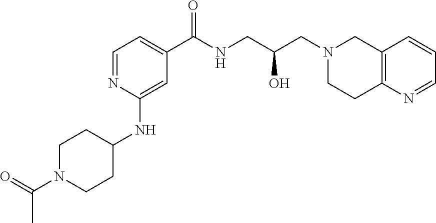 [SMILES: CC(=O)N1CCC(Nc2cc(C(=O)NC[C@H](O)CN3CCc4ncccc4C3)ccn2)CC1]

To a solution of 2-((1-acetylpiperidin-4-yl)amino)isonicotinic acid (329 mg, 1.25 mmol) in DCM (20 mL) was added Et₃N (253 mg, 2.5 mmol), HATU (712 mg, 1.9 mmol) and (S)-1-amino-3-(7,8-dihydro-1,6-naphthyridin-6(5H)-yl)propan-2-ol (300 mg, 1.5 mmol). The mixture was stirred at 23° C. for 16 h. TLC analysis showed the reaction complete and the solution was concentrated. The residue was then purified by Prep-HPLC to give the title compound (40 mg, 7.0%). ¹H NMR (CD₃OD, 400 MHz) δ (ppm): 8.60 (br. s., 1H), 7.99 (d, J=6.8 Hz, 1H), 7.91 (br. s., 1H), 7.58-7.42 (m, 2H), 7.22 (d, J=6.5 Hz, 1H), 4.68 (br. s., 2H), 4.53 (d, J=13.6 Hz, 1H), 4.41 (d, J=5.3 Hz, 1H), 4.06-3.91 (m, 2H), 3.84 (br. s., 2H), 3.65-3.45 (m, 3H), 3.45-3.35 (m, 4H), 2.95-2.86 (m, 1H), 2.24-2.03 (m, 5H), 1.71-1.46 (m, 2H). LCMS (m/z): 453.3 [M+H]⁺

### Example 48: (S)-2-((1-acetylpiperidin-4-yl)amino)-N-(3-(6,7-dihydro-1H-imidazo[4,5-c]pyridin-5(4H)-yl)-2-hydroxypropyl)isonicotinamide (Compound 3-36)

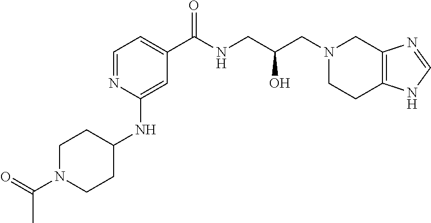 [SMILES: CC(=O)N1CCC(Nc2cc(C(=O)NC[C@H](O)CN3CCc4[nH]cnc4C3)ccn2)CC1]

To a stirring solution of 2-((1-acetylpiperidin-4-yl)amino)isonicotinic acid (150 mg, 0.57 mmol) in DCM (10 mL) was added HATU (216.6 mg, 0.57 mmol) and TEA (172.7 mg, 1.71 mmol) at 25° C. The solution was stirred at this temperature for 10 min, before (S)-1-amino-3-(3-((2-(trimethylsilyl)ethoxy)methyl)-6,7-dihydro-3H-imidazo[4,5-c]pyridin-5 (4H)-yl)propan-2-ol (185.8 mg, 0.57 mmol) was added. The mixture was stirred at 25° C. for another 3 h. Next, the mixture was taken up with DCM (50 mL), washed with H₂O (20 mL). The DCM layer was evaporated to dryness and the residue was re-dissolved in DCM (10 mL) and TFA (2 mL) was added. The mixture was stirred at 25° C. for 16 h. After that, the mixture was evaporated and the residue was purified by prep-HPLC to give the desired product (17.9 mg, yield: 7.1%). 1H NMR (400 MHz, CD₃OD-d₄): δ (ppm)=8.59 (s, 1H), 7.98 (d, J=6.8 Hz, 1H), 7.41 (s, 1H), 7.16 (d, J=6.5 Hz, 1H), 4.50 (br. s., 3H), 4.32 (d, J=5.5 Hz, 1H), 4.03-3.90 (m, 2H), 3.70 (br. s., 2H), 3.62-3.34 (m, 5H), 3.16-3.07 (m, 2H), 2.89 (t, J=11.5 Hz, 1H), 2.18-2.01 (m, 5H), 1.65-1.45 (m, 2H). LCMS (m/z): 442.3 [M+H]⁺

### Example 49: (S)-6-((1-acetylazetidin-3-yl)amino)-N-(2-hydroxy-3-(1-methyl-6,7-dihydro-1H-pyrazolo[4,3-c]pyridin-5(4H)-yl)propyl)pyrimidine-4-carboxamide (Compound 3-31)

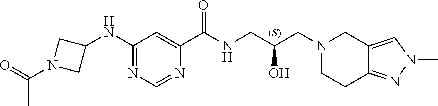

### Step 1: (Z)-tert-butyl 3-((dimethylamino)methylene)-4-oxopiperidine-1-carboxylate

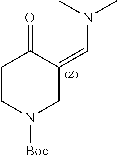 [SMILES: CN(C)/C=C1/CN(C(=O)OC(C)(C)C)CCC1=O]

A solution of tert-butyl 4-oxopiperidine-1-carboxylate (15 g, 75.4 mmol) in DMF-DMA (100 mL) was stirred at 120° C. for 4 h. The reaction mixture was cooled and concentrated to remove the DMF-DMA. The residue was used then directly in the next step without further purification. LCMS (m/z): 255.2 [M+H]⁺.

### Step 2: tert-butyl 6,7-dihydro-1H-pyrazolo[4,3-c]pyridine-5(4H)-carboxylate

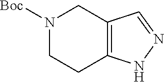 [SMILES: CC(C)(C)OC(=O)N1CCc2[nH]ncc2C1]

To a solution of (Z)-tert-butyl 3-((dimethylamino)methylene)-4-oxopiperidine-1-carboxylate (8.8 g, 34.6 mmol) in EtOH (50 mL) was added N₂H₄/H₂O (200 mL) at 20° C. The mixture was stirred at 20° C. for 12 h. After that, the reaction mixture was concentrated in vacuum. The residue mixture was purified with column separation to afford desired product (7 g, Yield 95%). LCMS (m/z): 224.2 [M+H]⁺.

### Step 3: tert-butyl 1-methyl-1,4,6,7-tetrahydro-5H-pyrazolo[4,3-c]pyridine-5-carboxylate and tert-butyl 2-methyl-2,4,6,7-tetrahydro-5H-pyrazolo[4,3-c]pyridine-5-carboxylate

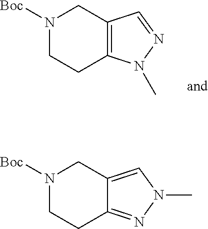 [SMILES: Cn1ncc2c1CCN(C(=O)OC(C)(C)C)C2 ; Cn1cc2c(n1)CCN(C(=O)OC(C)(C)C)C2]

To a stirred solution of tert-butyl 6,7-dihydro-1H-pyrazolo[4,3-c]pyridine-5(4H)-carboxylate (4 g, 17.9 mmol) in THF (100 mL) was added NaH (859 mg, 35.8 mmol) at 0° C. The mixture was stirred at 0° C. for 2 h, then MeI (5 g, 35.8 mmol) was added dropwise at 0° C. The mixture was stirred at 0° C. for 3 h, and then the 50 mL of H₂O was added dropwise to the mixture. The resulting mixture was extracted with DCM (100 mL×3) and the combined organic layer was concentrated under reduce pressure to give the crude mixture of products as yellow solid (4 g, crude), which was used in next step without further purification. LCMS (m/z): 238.3 [M+H]+.

### Step 4: 1-methyl-4,5,6,7-tetrahydro-1H-pyrazolo[4,3-c]pyridine and 2-methyl-4,5,6,7-tetrahydro-2H-pyrazolo[4,3-c]pyridine

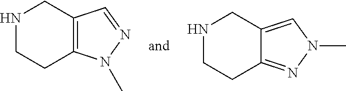

To a solution of tert-butyl 1-methyl-1,4,6,7-tetrahydro-5H-pyrazolo[4,3-c]pyridine-5-carboxylate and tert-butyl 2-methyl-2,4,6,7-tetrahydro-5H-pyrazolo[4,3-c]pyridine-5-carboxylate (4 g, crude) in DCM (40 mL) was added HCl/MeOH (10 mL) dropwise at 0° C. After addition, the mixture was warmed up to 20° C. slowly, and the stirring was continued for 3 h. The solid was precipitate and collected by filtration to give the crude product mixture as a yellow solid (2.8 g, crude). LCMS (m/z): 138.2 [M+H]⁺.

### Step 5: (R)-1-methyl-5-(oxiran-2-ylmethyl)-4,5,6,7-tetrahydro-1H-pyrazolo[4,3-c]pyridine and (R)-2-methyl-5-(oxiran-2-ylmethyl)-4,5,6,7-tetrahydro-2H-pyrazolo[4,3-c]pyridine

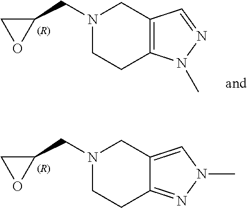

To a mixture of 1-methyl-4,5,6,7-tetrahydro-1H-pyrazolo[4,3-c]pyridine and 2-methyl-4,5,6,7-tetrahydro-2H-pyrazolo[4,3-c]pyridine (2.8 g, crude) and KF (3.1 g, 53.7 mmol) in THF (200 mL) was added (S)-oxiran-2-ylmethyl 3-nitrobenzenesulfonate (9.2 g, 35.8 mmol) at 20° C. The mixture was stirred at 40° C. for 16 h, at which time LCMS showed the completion of the reactions. The mixture was filtered and concentrated to give the crude mixed product (4.9 g, crude), which was used in next step without further purification. LCMS (m/z): 194.2 [M+H]⁺.

### Step 6: (S)-1-amino-3-(1-methyl-1,4,6,7-tetrahydro-5H-pyrazolo[4,3-c]pyridin-5-yl)propan-2-ol and (S)-1-amino-3-(2-methyl-2,4,6,7-tetrahydro-5H-pyrazolo[4,3-c]pyridin-5-yl)propan-2-ol

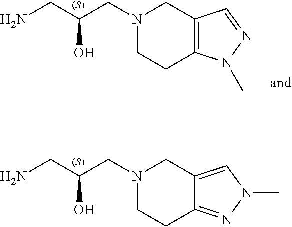

To a mixture of (R)-1-methyl-5-(oxiran-2-ylmethyl)-4,5,6,7-tetrahydro-1H-pyrazolo[4,3-c]pyridine and (R)-2-methyl-5-(oxiran-2-ylmethyl)-4,5,6,7-tetrahydro-2H-pyrazolo[4,3-c]pyridine (4.9 g, crude) in EtOH (50 mL) was added NH₃/H₂O (100 mL) at 20° C. The mixture was stirred at 40° C. for 12 h, at which time LCMS showed the completion of the reactions. The mixture was concentrated to give the crude mixed product (1.16 g, crude), which was used in next step without further purification. LCMS (m/z): 211.2 [M+H]⁺.

### Step 7: (S)-6-chloro-N-(2-hydroxy-3-(1-methyl-6,7-dihydro-1H-pyrazolo[4,3-c]pyridin-5(4H)-yl)propyl)pyrimidine-4-carboxamide and (S)-6-chloro-N-(2-hydroxy-3-(2-methyl-2,4,6,7-tetrahydro-5H-pyrazolo[4,3-c]pyridin-5-yl)propyl)pyrimidine-4-carboxamide

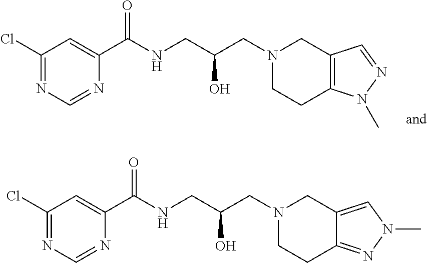 [SMILES: Cn1ncc2c1CCN(C[C@@H](O)CNC(=O)c1cc(Cl)ncn1)C2 ; Cn1cc2c(n1)CCN(C[C@@H](O)CNC(=O)c1cc(Cl)ncn1)C2]

To a mixture of (S)-1-amino-3-(1-methyl-1,4,6,7-tetrahydro-5H-pyrazolo[4,3-c]pyridin-5-yl)propan-2-ol and (S)-1-amino-3-(2-methyl-2,4,6,7-tetrahydro-5H-pyrazolo[4,3-c]pyridin-5-yl)propan-2-ol (1.16 g, crude) in DCM (40 mL) was added Et₃N (2 mL) at 17° C. The mixture was stirred at 17° C. for 0.5 h, and then 6-chloropyrimidine-4-carbonyl chloride (800 mg, 4.5 mmol) was added at 17° C. LCMS showed the reaction completed, the reaction mixture was diluted with water (50 mL), extracted with DCM (100 mL×3). The combined organic layer was concentrated to give the crude product which were then purified by column chromatography on silica gel to give the mixture of title compounds as a yellow oil (1.2 g, 75.9%). LCMS (m/z): 351.2 [M+H]⁺.

### Step 8: (S)-6-((1-acetylazetidin-3-yl)amino)-N-(2-hydroxy-3-(1-methyl-6,7-dihydro-1H-pyrazolo[4,3-c]pyridin-5(4H)-yl)propyl)pyrimidine-4-carboxamide

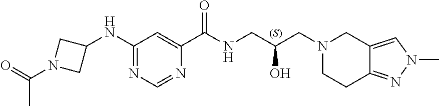

To a mixture of (S)-6-chloro-N-(2-hydroxy-3-(1-methyl-6,7-dihydro-1H-pyrazolo[4,3-c]pyridin-5(4H)-yl)propyl)pyrimidine-4-carboxamide and (S)-6-chloro-N-(2-hydroxy-3-(2-methyl-2,4,6,7-tetrahydro-5H-pyrazolo [4,3-c]pyridin-5-yl)propyl)pyrimidine-4-carboxamide (350 mg, 1 mmol) and 1-(3-aminoazetidin-1-yl)ethanone (171 mg, 1.5 mmol) in i-PrOH (20 mL) was added Et₃N (1 mL) at 25° C. The mixture was stirred at 80° C. for 12 h. LCMS showed the completion of the reactions, the reaction mixture was concentrated to give a crude mixture of products. The title compound (S)-6-((1-acetylazetidin-3-yl)amino)-N-(2-hydroxy-3-(1-methyl-6,7-dihydro-1H-pyrazolo [4,3-c]pyridin-5 (4H)-yl)propyl)pyrimidine-4-carboxamide was isolated after purification by prep-HPLC first and then further purification by SFC as white solid (78 mg, 18.2%). 1H NMR (CD3OD, 400 MHz) δ (ppm): 8.41 (s, 1H) 7.25-7.32 (m, 1H) 7.16 (br. s., 1H) 4.77 (br. s., 1H) 4.59 (t, J=8.41 Hz, 1H) 4.36 (t, J=9.03 Hz, 1H) 4.02-4.12 (m, 2H) 3.90 (dd, J=10.16, 5.14 Hz, 1H) 3.82 (s, 3H) 3.63 (s, 2H) 3.46-3.57 (m, 2H) 2.93 (tq, J=11.29, 5.94 Hz, 2H) 2.76-2.83 (m, 2H) 2.70 (d, J=6.02 Hz, 2H) 1.91 (s, 3H). LCMS (m/z): 429.2 [M+H]⁺.

### Biological Assays

### PRMT5 Biochemical Assay

General Materials.

S-adenosylmethionine (SAM), S-adenosylhomocysteine (SAH), bicine, KCl, Tween20, dimethylsulfoxide (DMSO), bovine skin gelatin (BSG), and Tris(2-carboxyethyl)phosphine hydrochloride solution (TCEP) were purchased from Sigma-Aldrich at the highest level of purity possible. ³H-SAM was purchase from American Radiolabeled Chemicals with a specific activity of 80 Ci/mmol. 384-well streptavidin Flashplates were purchased from PerkinElmer.

Substrates.

Peptide representative of human histone H4 residues 1-15 was synthesized with a C-terminal linker-affinity tag motif and a C-terminal amide cap by 21^{st} Century Biochemicals. The peptide was high high-performance liquid chromatography (HPLC) purified to greater than 95% purity and confirmed by liquid chromatography mass spectrometry (LC-MS). The sequence was Ac-SGRGKGGKGLGKGGA[K-Biot]-amide (SEQ ID NO.:3).

Molecular Biology:

Full-length human PRMT5 (NM_006109.3) transcript variant 1 clone was amplified from a fetal brain cDNA library, incorporating flanking 5′ sequence encoding a FLAG tag (MDYKDDDDK) (SEQ ID NO.:4) fused directly to Ala 2 of PRMT5. Full-length human MEP50 (NM_024102) clone was amplified from a human testis cDNA library incorporating a 5′ sequence encoding a 6-histidine tag (MHHHHHH) (SEQ ID NO.:5) fused directly to Arg 2 of MEP50. The amplified genes were sublconed into pENTR/D/TEV (Life Technologies) and subsequently transferred by Gateway™ attL×attR recombination to pDEST8 baculvirus expression vector (Life Technologies).

Protein Expression.

Recombinant baculovirus and Baculovirus-Infected Insect Cells (BIIC) were generated according to Bac-to-Bac kit instructions (Life Technologies) and Wasilko, 2006, respectively. Protein over-expression was accomplished by infecting exponentially growing Spodoptera frugiperda (SF9) cell culture at 1.2×10⁶ cell/ml with a 5000 fold dilution of BIIC stock. Infections were carried out at 27° C. for 72 hours, harvested by centrifugation, and stored at −80° C. for purification.

Protein Purification.

Expressed full-length human Flag-PRMT5/6His-MeP50 protein complex was purified from cell paste by NiNTA agarose affinity chromatography after a five hour equilibration of the resin with buffer containing 50 mM Tris-HCL, pH 8.0, 25 mM NaCl, and 1 mM TCEP at 4° C., to minimize the adsorption of tubulin impurity by the resin. Flag-PRMT5/6His-MeP50 was eluted with 300 mM Imidazole in the same buffer. The purity of recovered protein was 87%. Reference: Wasilko, D. J. and S. E. Lee: “TIPS: titerless infected-cells preservation and scale-up” Bioprocess J., 5 (2006), pp. 29-32.

Predicted Translations:

Flag-PRMT5
(SEQ ID NO.: 6)
MDYKDDDDKA AMAVGGAGGS RVSSGRDLNC VPEIADTLGA
VAKQGFDFLC MPVFHPRFKR EFIQEPAKNR PGPQTRSDLL
LSGRDWNTLI VGKLSPWIRP DSKVEKIRRN SEAAMLQELN
FGAYLGLPAF LLPLNQEDNT NLARVLTNHI HTGHHSSMFW
MRVPLVAPED LRDDIIENAP TTHTEEYSGE EKTWMWWHNF
RTLCDYSKRI AVALEIGADL PSNHVIDRWL GEPIKAAILP
TSIFLTNKKG FPVLSKMHQR LIFRLLKLEV QFIITGTNHH
SEKEFCSYLQ YLEYLSQNRP PPNAYELFAK GYEDYLQSPL
QPLMDNLESQ TYEVFEKDPI KYSQYQQAIY KCLLDRVPEE
EKDTNVQVLM VLGAGRGPLV NASLRAAKQA DRRIKLYAVE
KNPNAVVTLE NWQFEEWGSQ VTVVSSDMRE WVAPEKADII
VSELLGSFAD NELSPECLDG AQHFLKDDGV SIPGEYTSFL
APISSSKLYN EVRACREKDR DPEAQFEMPY VVRLHNFHQL
SAPQPCFTFS HPNRDPMIDN NRYCTLEFPV EVNTVLHGFA
GYFETVLYQD ITLSIRPETH SPGMFSWFPI LFPIKQPITV
REGQTICVRF WRCSNSKKVW YEWAVTAPVC SAIHNPTGRS
YTIG L
6His-MEP50
(SEQ ID NO.: 7)
MHHHHHHRKE TPPPLVPPAA REWNLPPNAP ACMERQLEAA
RYRSDGALLL GASSLSGRCW AGSLWLFKDP CAAPNEGFCS
AGVQTEAGVA DLTWVGERGI LVASDSGAVE LWELDENETL
IVSKFCKYEH DDIVSTVSVL SSGTQAVSGS KDICIKVWDL
AQQVVLSSYR AHAAQVTCVA ASPHKDSVFL SCSEDNRILL
WDTRCPKPAS QIGCSAPGYL PTSLAWHPQQ SEVFVFGDEN
GTVSLVDTKS TSCVLSSAVH SQCVTGLVFS PHSVPFLASL
SEDCSLAVLD SSLSELFRSQ AHRDFVRDAT WSPLNHSLLT
TVGWDHQVVH HVVPTEPLPA PGPASVTE

General Procedure for PRMT5/MEP50 Enzyme Assays on Peptide Substrates.

The assays were all performed in a buffer consisting of 20 mM Bicine (pH=7.6), 1 mM TCEP, 0.005% BSG, and 0.002% Tween20, prepared on the day of use. Compounds in 100% DMSO (1 ul) were spotted into a polypropylene 384-well V-bottom plates (Greiner) using a Platemate Plus outfitted with a 384-channel head (Thermo Scientific). DMSO (1 ul) was added to Columns 11, 12, 23, 24, rows A-H for the maximum signal control and 1 ul of SAH, a known product and inhibitor of PRMT5/MEP50, was added to columns 11, 12, 23, 24, rows I-P for the minimum signal control. A cocktail (40 ul) containing the PRMT5/MEP50 enzyme and the peptide was added by Multidrop Combi (Thermo-Fisher). The compounds were allowed to incubate with PRMT5/MEP50 for 30 min at 25 degrees Celsius, then a cocktail (10 ul) containing ³H-SAM was added to initiate the reaction (final volume=51 ul). The final concentrations of the components were as follows: PRMT5/MEP50 was 4 nM, ³H-SAM was 75 nM, peptide was 40 nM, SAH in the minimum signal control wells was 100 uM, and the DMSO concentration was 1%. The assays were stopped by the addition of non-radioactive SAM (10 ul) to a final concentration of 600 uM, which dilutes the ³H-SAM to a level where its incorporation into the peptide substrate is no longer detectable. 50 ul of the reaction in the 384-well polypropylene plate was then transferred to a 384-well Flashplate and the biotinylated peptides were allowed to bind to the streptavidin surface for at least 1 hour before being washed three times with 0.1% Tween20 in a Biotek ELx405 plate washer. The plates were then read in a PerkinElmer TopCount plate reader to measure the quantity of ³H-labeled peptide bound to the Flashplate surface, measured as disintegrations per minute (dpm) or alternatively, referred to as counts per minute (cpm).

% inhibition calculation

$$\% inh = 100 - ( \frac{{dpm}_{cmpd} - {dpm}_{m i n}}{{dpm}_{ma x} - {dpm}_{m i n}} ) \times 100$$

Where dpm=disintegrations per minute, cmpd=signal in assay well, and min and max are the respective minimum and maximum signal controls.

### Four-parameter IC50 fit

$$Y = Bottom + \frac{( Top - Bottom )}{( 1 + {( \frac{X}{{IC}_{50}} )}^{Hill Coefficient}}$$

Where top and bottom are the normally allowed to float, but may be fixed at 100 or 0 respectively in a 3-parameter fit. The Hill Coefficient normally allowed to float but may also be fixed at 1 in a 3-parameter fit. Y is the % inhibition and X is the compound concentration.

### Z-138 Methylation Assay

Z-138 suspension cells were purchased from ATCC (American Type Culture Collection, Manassas, Va.). RPMI/Glutamax medium, penicillin-streptomycin, heat inactivated fetal bovine serum, and D-PBS were purchased from Life Technologies, Grand Island, N.Y., USA. Odyssey blocking buffer, 800CW goat anti-rabbit IgG (H+L) antibody, and Licor Odyssey infrared scanner were purchased from Licor Biosciences, Lincoln, Nebr., USA. Symmetric di-methyl arginine antibody was purchased from EMD Millipore, Billerica, Mass., USA. 16% Paraformaldehyde was purchased from Electron Microscopy Sciences, Hatfield, Pa., USA.

Z-138 suspension cells were maintained in growth medium (RPMI 1640 supplemented with 10% v/v heat inactivated fetal bovine serum and 100 units/mL penicillin-streptomycin) and cultured at 37° C. under 5% CO₂.

Cell Treatment, In Cell Western (ICW) for detection of Symmetric di-Methyl Arginine and DNA content.

Z-138 cells were seeded in assay medium at a concentration of 50,000 cells per mL to a 384-well cell culture plate with 50 μL per well. Compound (100 nL) from 384 well source plates was added directly to 384 well cell plate. Plates were incubated at 37° C., 5% CO₂ for 96 hours. After four days of incubation, 40 μL of cells from incubated plates were added to poly-D-lysine coated 384 well culture plates (BD Biosciences 356697). Plates were incubated at room temperature for 30 minutes then incubated at 37° C., 5% CO₂ for 5 hours. After the incubation, 40 μL per well of 8% paraformaldehyde in PBS (16% paraformaldahyde was diluted to 8% in PBS) was added to each plate and incubated for 30 minutes. Plates were transferred to a Biotek 405 plate washer and washed 5 times with 100 μL per well of wash buffer (IX PBS with 0.1% Triton X-100 (v/v)). Next 30 pLper well of Odyssey blocking buffer were added to each plate and incubated 1 hour at room temperature. Blocking buffer was removed and 20 μL per well of primary antibody was added (symmetric di-methyl arginine diluted 1:100 in Odyssey buffer with 0.1% Tween 20 (v/v)) and plates were incubated overnight (16 hours) at 4° C. Plates were washed 5 times with 100 μL per well of wash buffer. Next 20 μL per well of secondary antibody was added (1:200 800CW goat anti-rabbit IgG (H+L) antibody, 1:1000 DRAQ5 (Biostatus limited) in Odyssey buffer with 0.1% Tween 20 (v/v)) and incubated for 1 hour at room temperature. The plates were washed 5 times with 100 μL per well wash buffer then 1 time with 100 μL per well of water. Plates were allowed to dry at room temperature then imaged on the Licor Odyssey machine which measures integrated intensity at 700 nm and 800 nm wavelengths. Both 700 and 800 channels were scanned.

Calculations:

First, the ratio for each well was determined by:

$$( \frac{symmetric di \text{-} methyl Arginine 800 nm value}{DRAQ 5 \; 700 nm value} )$$

Each plate included fourteen control wells of DMSO only treatment (minimum inhibition) as well as fourteen control wells for maximum inhibition treated with 3 μM of a reference compound (Background wells). The average of the ratio values for each control type was calculated and used to determine the percent inhibition for each test well in the plate. Reference compound was serially diluted three-fold in DMSO for a total of nine test concentrations, beginning at 3 μM. Percent inhibition was determined and IC₅₀ curves were generated using triplicate wells per concentration of compound.

$$Percent Inhibition = 100 - ( ( \frac{( Individual Test Sample Ratio ) - ( Background Avg Ratio )}{( Minimum Inhibition Ratio ) - ( Background Average Ratio )} ) * 100 )$$

### Z-138 Proliferation Assay

Z-138 suspension cells were purchased from ATCC (American Type Culture Collection, Manassas, Va.). RPMI/Glutamax medium, penicillin-streptomycin, heat inactivated fetal bovine serum were purchased from Life Technologies, Grand Island, N.Y., USA. V-bottom polypropylene 384-well plates were purchased from Greiner Bio-One, Monroe, N.C., USA. Cell culture 384-well white opaque plates were purchased from Perkin Elmer, Waltham, Mass., USA. Cell-Titer Glo® was purchased from Promega Corporation, Madison, Wis., USA. SpectraMax M5 plate reader was purchased from Molecular Devices LLC, Sunnyvale, Calif., USA.

Z-138 suspension cells were maintained in growth medium (RPMI 1640 supplemented with 10% v/v heat inactivated fetal bovine serum and cultured at 37° C. under 5% CO₂. Under assay conditions, cells were incubated in assay medium (RPMI 1640 supplemented with 10% v/v heat inactivated fetal bovine serum and 100 units/mL penicillin-streptomycin) at 37° C. under 5% CO₂.

For the assessment of the effect of compounds on the proliferation of the Z-138 cell line, exponentially growing cells were plated in 384-well white opaque plates at a density of 10,000 cells/ml in a final volume of 50 μl of assay medium. A compound source plate was prepared by performing triplicate nine-point 3-fold serial dilutions in DMSO, beginning at 10 mM (final top concentration of compound in the assay was 20 μM and the DMSO was 0.2%). A 100 nL aliquot from the compound stock plate was added to its respective well in the cell plate. The 100% inhibition control consisted of cells treated with 200 nM final concentration of staurosporine and the 0% inhibition control consisted of DMSO treated cells. After addition of compounds, assay plates were incubated for 5 days at 37° C., 5% CO₂, relative humidity >90%.

Cell viability was measured by quantitation of ATP present in the cell cultures, adding 35 μl of Cell Titer Glo® reagent to the cell plates. Luminescence was read in the SpectraMax M5 microplate reader. The concentration of compound inhibiting cell viability by 50% was determined using a 4-parametric fit of the normalized dose response curves.

Results for certain compounds described herein are shown in Table 2.

TABLE 2
Biological Assay Results

| Cmpd No | Biochemical IC₅₀ | ICW EC₅₀ | Proliferation EC₅₀ |
|---|---|---|---|
| 1-1 | A | A | C |
| 1-2 | A | A | B |
| 1-3 | A | A | B |
| 1-4 | A | A | C |
| 1-5 | A | B | G |
| 1-6 | C | — | G |
| 1-7 | C | F | G |
| 1-8 | * | F | G |
| 1-9 | C | — | — |
| 1-10 | A | — | — |
| 1-11 | B | A | C |
| 1-12 | A | A | B |
| 1-13 | A | A | B |
| 1-14 | A | A | B |
| 1-15 | A | — | — |
| 1-16 | * | F | G |
| 1-17 | * | F | G |
| 1-18 | C | F | G |
| 1-19 | C | F | G |
| 1-20 | * | F | G |
| 1-21 | * | — | — |
| 1-22 | * | — | — |
| 1-23 | * | F | G |
| 1-24 | A | — | — |
| 1-25 | * | — | — |
| 1-26 | B | — | — |
| 1-27 | C | F | G |
| 1-28 | C | F | G |
| 1-29 | C | F | G |
| 1-30 | C | B | G |
| 1-31 | A | — | — |
| 1-32 | C | F | G |
| 1-33 | C | F | G |
| 1-34 | B | F | G |
| 1-35 | B | F | G |
| 1-36 | C | F | G |
| 1-37 | C | F | G |
| 1-38 | C | F | G |
| 1-39 | A | — | — |
| 2-1 | E | — | *** |
| 2-2 | E | — | — |
| 2-3 | E | — | — |
| 2-4 | D | — | — |
| 2-5 | C | F | G |
| 2-6 | C | F | G |
| 2-7 | C | F | G |
| 2-8 | C | F | G |
| 2-9 | B | B | G |
| 2-10 | C | — | C |
| 2-11 | B | B | G |
| 2-12 | B | G | C |
| 2-13 | B | B | C |
| 2-14 | A | B | C |
| 2-15 | C | F | G |
| 2-16 | B | F | G |
| 2-17 | B | F | G |
| 2-18 | B | B | B |
| 2-19 | B | F | G |
| 2-20 | B | B | G |
| 2-21 | B | B | G |
| 2-22 | C | F | G |
| 2-23 | C | F | G |
| 2-24 | C | F | G |
| 3-1 | E | — | — |
| 3-2 | E | — | — |
| 3-3 | E | — | — |
| 3-4 | E | — | — |
| 3-5 | E | — | — |
| 3-6 | D | — | — |
| 3-7 | E | — | — |
| 3-8 | E | — | — |
| 3-9 | D | — | D |
| 3-10 | * | F | G |
| 3-11 | C | F | G |
| 3-12 | * | F | G |
| 3-13 | E | — | — |
| 3-14 | E | — | *** |
| 3-15 | E | — | — |
| 3-16 | E | — | — |
| 3-17 | * | F | G |
| 3-18 | E | — | — |
| 3-19 | * | F | — |
| 3-20 | C | — | — |
| 3-21 | C | F | G |
| 3-22 | * | F | G |
| 3-23 | C | F | G |
| 3-24 | * | F | G |
| 3-25 | * | F | G |
| 3-26 | * | F | G |
| 3-27 | * | F | G |
| 3-28 | * | F | G |
| 3-29 | * | F | G |
| 3-30 | * | F | G |
| 3-31 | * | F | G |
| 3-32 | E | — | — |
| 3-33 | * | F | G |
| 3-34 | * | F | G |
| 3-35 | * | F | G |
| 3-36 | * | F | G |
| 3-37 | B | — | — |
| 3-38 | * | F | G |
| 3-39 | * | — | G |
| 3-40 | * | F | G |
| 3-41 | * | F | G |
| 3-42 | * | — | — |
| 3-43 | C | — | — |
| 3-44 | * | F | G |
| 3-45 | * | F | G |
| 3-46 | * | F | G |
| 3-47 | * | F | G |
| 3-48 | * | — | — |
| 3-49 | C | — | — |
| 3-50 | * | F | G |
| 3-51 | * | F | G |
| 3-52 | C | F | G |
| 3-53 | C | F | G |
| 3-54 | * | F | G |
| 3-55 | B | — | — |
| 3-56 | * | F | G |
| 3-57 | * | F | G |
| 3-58 | * | F | G |
| 3-59 | * | — | — |
| 3-60 | B | — | — |
| 4-1 | * | F | G |
| 4-2 | * | F | G |
| 4-3 | * | F | G |
| 4-4 | * | F | G |

“A” indicates an IC₅₀ or EC₅₀ < 0.100 μM
“B” indicates an IC₅₀ or EC₅₀ of 0.101-1.000 μM
“C” indicates an IC₅₀ or EC₅₀ of 1.001-10.000 μM
“D” indicates an IC₅₀ or EC₅₀ of 10.001-50 μM
“F” indicates an IC₅₀ or EC₅₀ > 1 μM
“G” indicates an IC₅₀ or EC₅₀ > 5 μM
“*” indicates an IC₅₀ or EC₅₀ > 10 μM
“**” indicates an IC₅₀ or EC₅₀ > 20 μM
“***” indicates an IC₅₀ or EC₅₀ > 40 μM
“E” indicates an IC₅₀ or EC₅₀ > 50 μM
“—” indicates no data

### OTHER EMBODIMENTS

The foregoing has been a description of certain non-limiting embodiments of the invention. Those of ordinary skill in the art will appreciate that various changes and modifications to this description may be made without departing from the spirit or scope of the present invention, as defined in the following claims.

What is claimed is:

1. A compound of Formula (A): 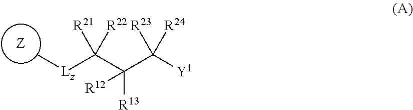
or a pharmaceutically acceptable salt thereof,
wherein:
R¹² is hydrogen, halogen, or optionally substituted C₁₋₃alkyl;
R¹³ is hydrogen, halogen, optionally substituted C₁₋₃alkyl, —NR^{A1}R^{A2}, or —OR¹;
R^{A1} and R^{A2} are each independently hydrogen, optionally substituted C₁₋₃ alkyl, a nitrogen protecting group, or R^{A1} and R^{A2} are taken together with the intervening nitrogen atom to form an optionally substituted 3-6 membered heterocyclic ring;
R¹ is hydrogen, R^{z}, or —C(O)R^{z}, wherein R^{z} is optionally substituted C₁₋₆ alkyl;
L_{z} is a linker or is absent;
Ring Z is an optionally substituted, monocyclic or bicyclic, saturated, partially unsaturated, or aromatic ring having 0-4 heteroatoms independently selected from nitrogen, oxygen, and sulfur;
R²¹, R²², R²³, and R²⁴ are independently hydrogen, halo, or optionally substituted aliphatic;
Y¹ is of formula (x) or formula (y) 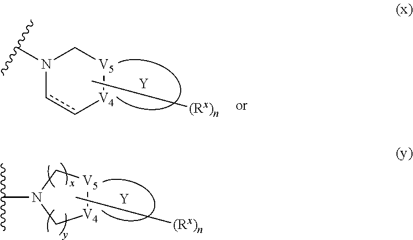
Ring Y is a 5- to 6-membered heteroaryl ring;
each instance of V₄ and V₅ is independently C or N;
each R^{x} is independently selected from the group consisting of halo, —CN, optionally substituted aliphatic, —OR′, —N(R″)₂, optionally substituted aryl, optionally substituted heteroaryl, and if attached to a nitrogen atom, a nitrogen protecting group;
R′ is hydrogen or optionally substituted aliphatic;
R″ is hydrogen or optionally substituted aliphatic, or two R″ are taken together with their intervening atoms to form a heterocyclic ring;
n is 0, 1, 2, 3, 4, 5, 6, 7, or 8; [symbol]
corresponds to a single or double bond; and
x is 0 and y is 2, 3, or 4; or
x is 1 and y is 1; or
x is 1 and y is 3.

2. The compound of claim 1, wherein Y¹ is of formula (x-1) 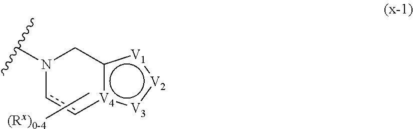
wherein:
each instance of V₁, V₂, and V₃ is independently O, S, N, NH, NR^{x}, CH, or CR^{x}; and
V₄ is N or C.

3. The compound of claim 1, wherein Y¹ is of formula (y-1) 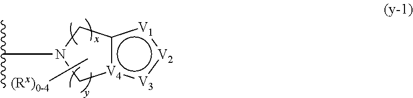
wherein:
each instance of V₁, V₂, and V₃ is independently O, S, N, NH, NR^{x}, CH, or CR^{x}; and
V₄ is N or C.

4. The compound of claim 3, wherein Y¹ is of formula (x-1a):

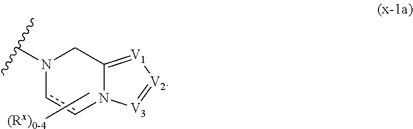

5. The compound of claim 4, wherein Y¹ is of formula (x-1b):

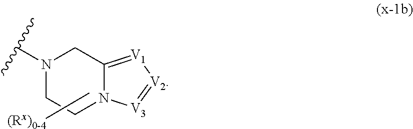

6. The compound of claim 5, wherein Y¹ is of formula (x-1c):

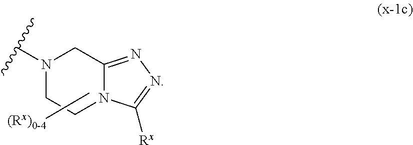

7. The compound of claim 1, wherein Y¹ is of formula (i), (ii), or (iii): 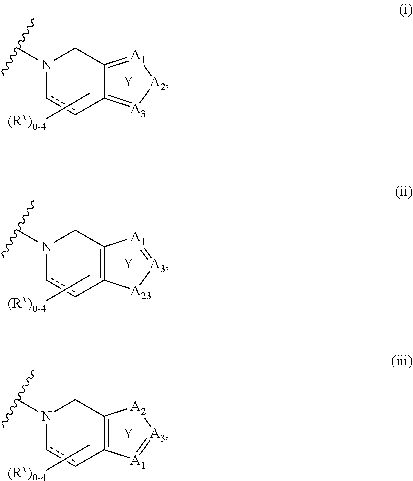
wherein:
each instance of A₁ and A₃ is independently N, CH, or CR^{x}; and
A₂ is O, S, NH, or NR^{x}.

8. The compound of claim 7, wherein Y¹ is of formula (i-a), (ii-a), or (iii-a):

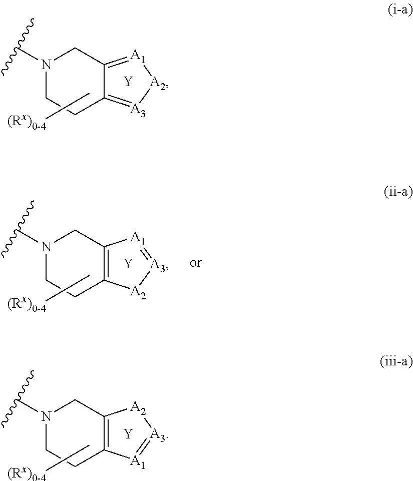

9. The compound of claim 8, wherein Y¹ is selected from the group consisting of: 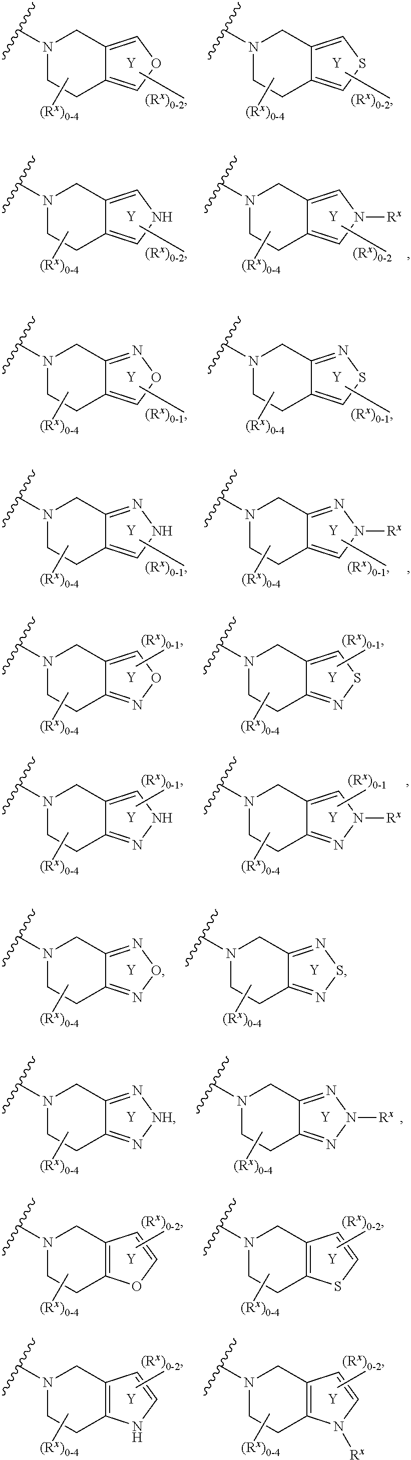 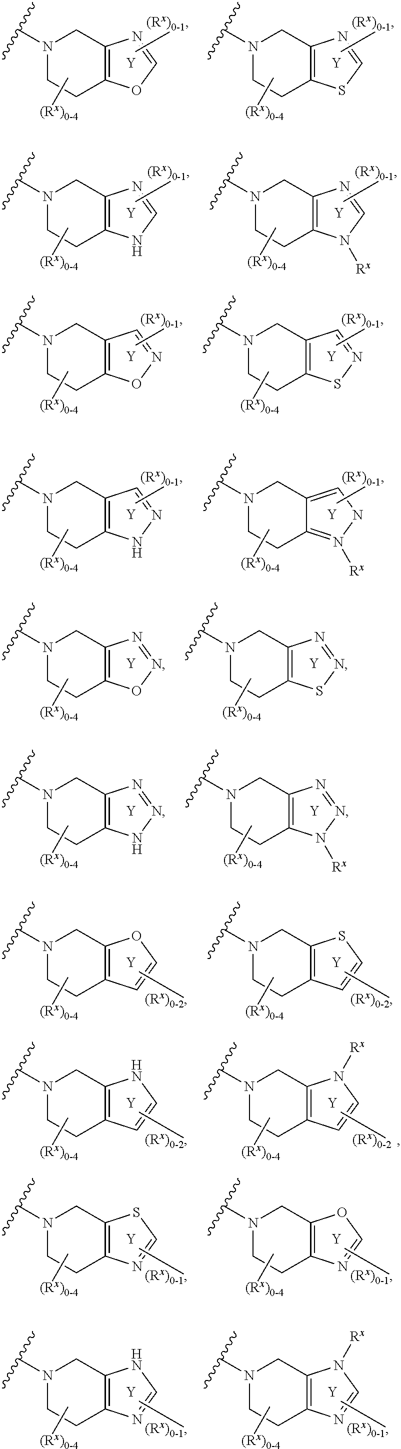 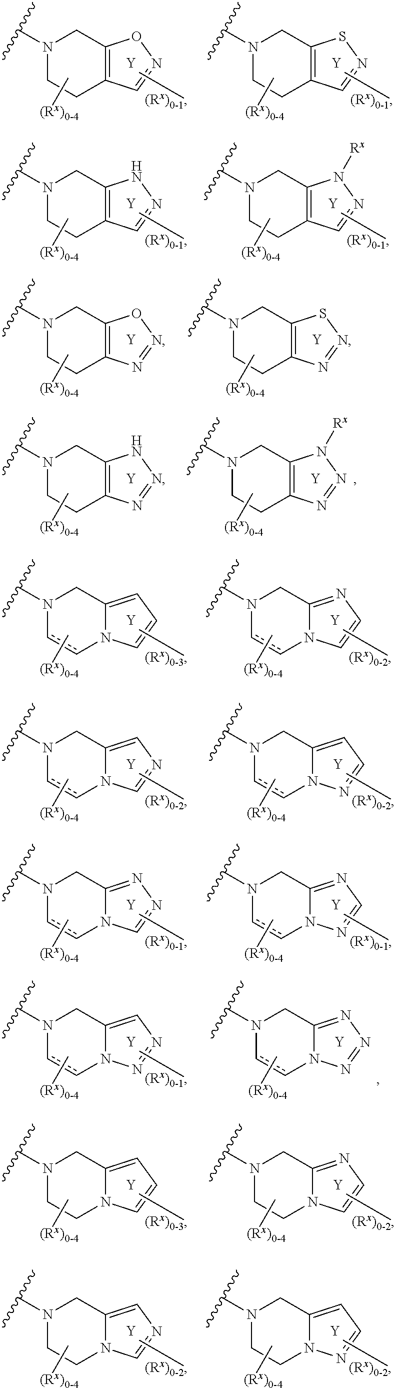 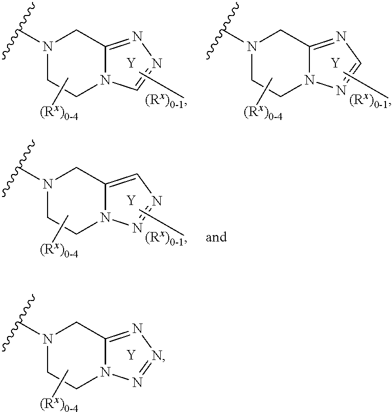
wherein the ring system fused to Ring Y comprises 0, 1, 2, 3, or 4 R^{x} substituents, and Ring Y comprises 0, 1, or 2 R^{x} substituents, as valency permits.

10. The compound of claim 1, wherein Y¹ is selected from the group consisting of: 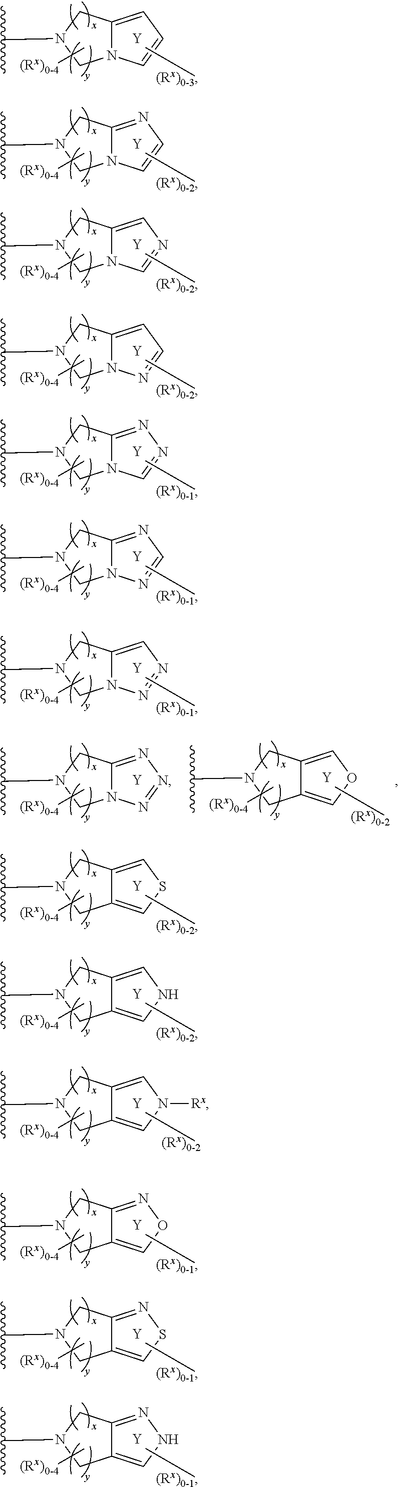 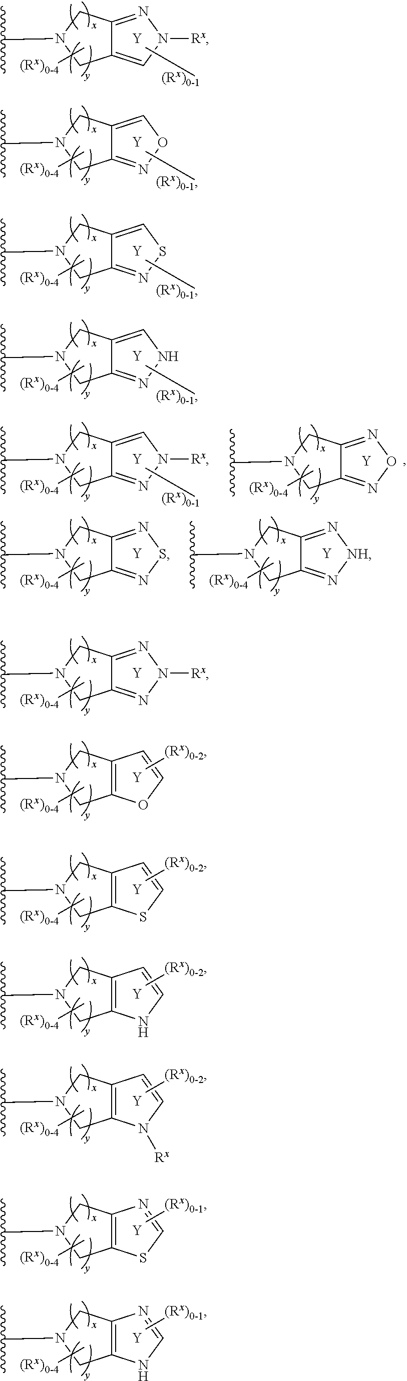 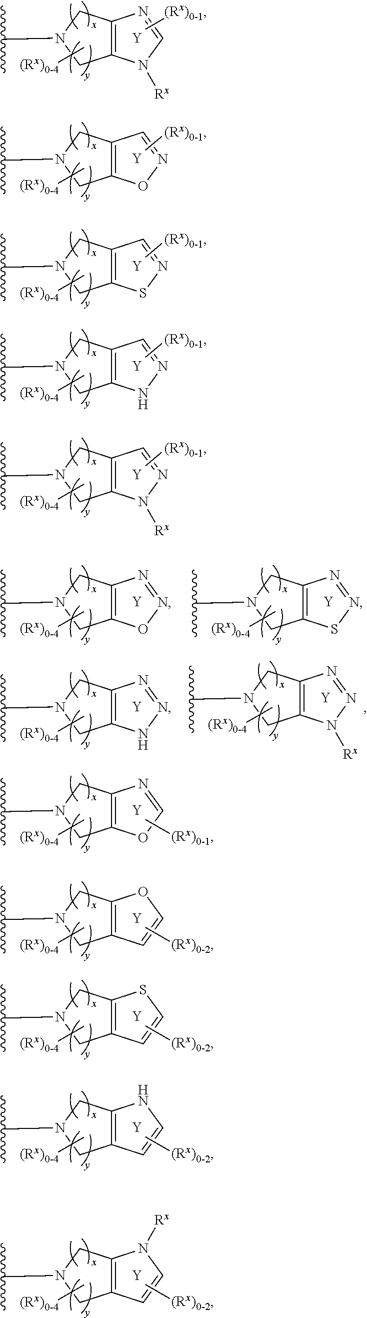 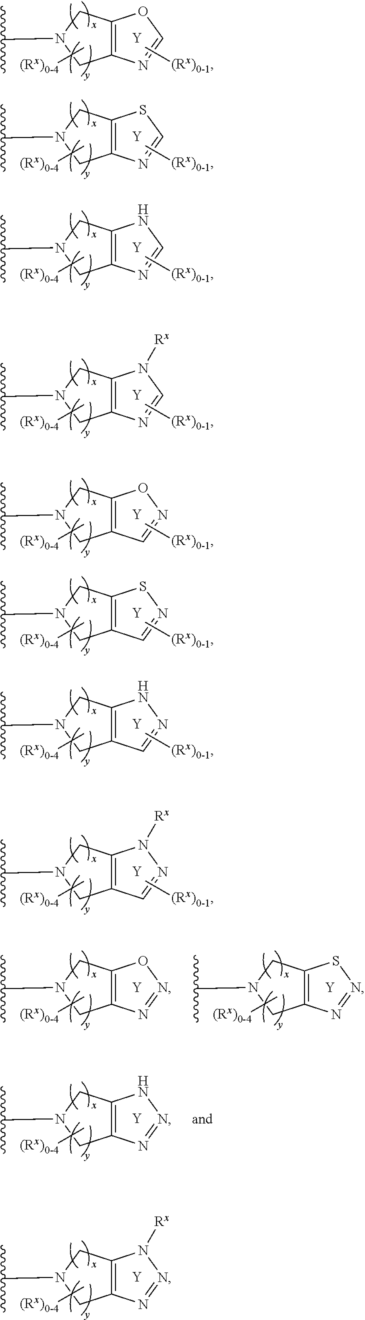
wherein the ring system fused to Ring Y comprises 0, 1, 2, 3, or 4 R^{x} substituents, and Ring Y comprises 0, 1, 2, or 3 R^{x} substituents, as valency permits.

11. The compound of claim 1, wherein Y¹ is of formula (iv): 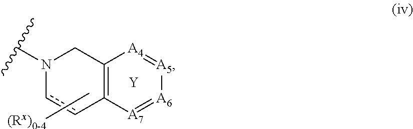
wherein each instance of A₄, A₅, A₆, and A₇ is independently N, CH, or CR^{x}, provided at least one of A₄, A₅, A₆, and A₇ is N.

12. The compound of claim 12, wherein Y¹ is of formula (iv-a):

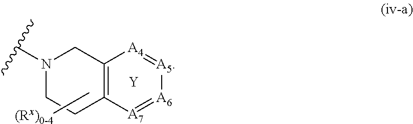

13. The compound of claim 13, wherein Y¹ is selected from the group consisting of: 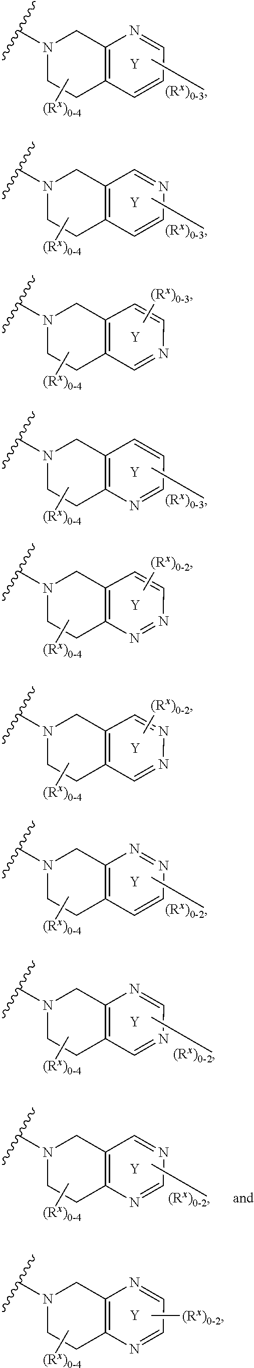
wherein the ring system fused to Ring Y comprises 0, 1, 2, 3, or 4 R^{x} substituents, and Ring Y comprises 0, 1, 2, or 3 R^{x} substituents, as valency permits.

14. The compound of claim 1, wherein Y¹ is selected from the group consisting of: 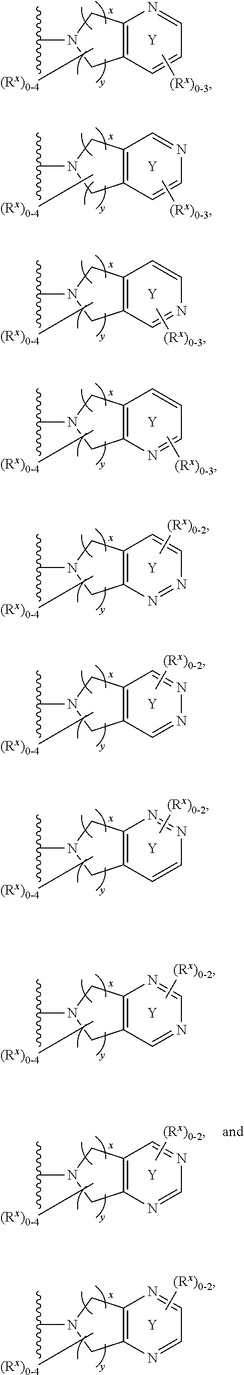
wherein the ring system fused to Ring Y comprises 0, 1, 2, 3, or 4 R^{x} substituents, and Ring Y comprises 0, 1, 2, or 3 R^{x} substituents, as valency permits.

15. The compound of claim 1, wherein the compound is of Formula: 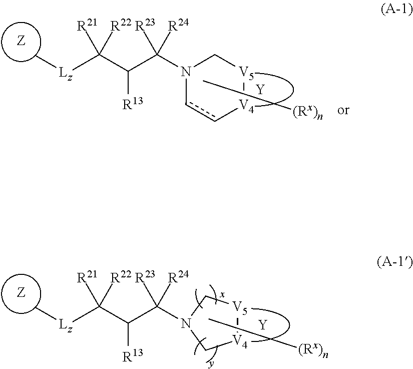
or a pharmaceutically acceptable salt thereof.

16. The compound of claim 1, wherein the compound is of Formula: 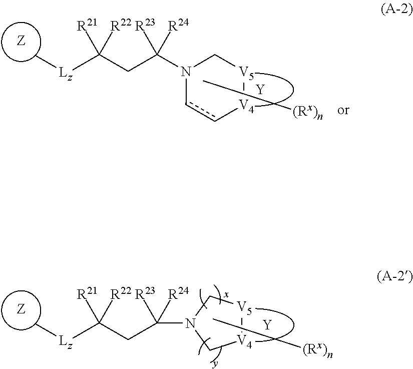
or a pharmaceutically acceptable salt thereof.

17. The compound of claim 1, wherein the compound is of Formula (A-3): 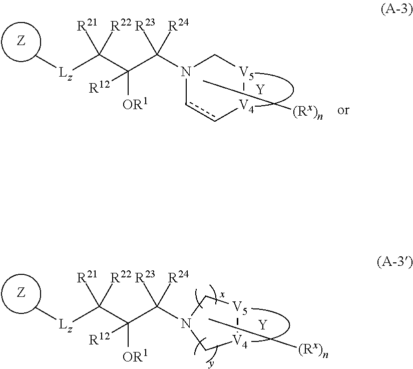
or a pharmaceutically acceptable salt thereof.

18. The compound of claim 1, wherein the compound is of Formula: 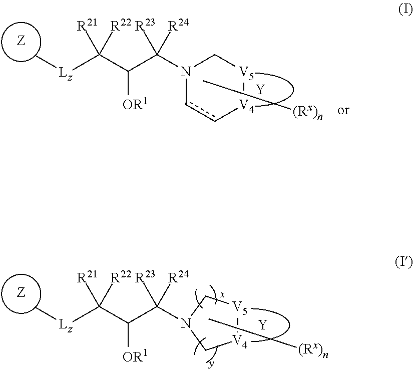
or a pharmaceutically acceptable salt thereof.

19. The compound of any one of claims 1-18, wherein L_{z} is a linker:
—X_{A}—C(R^{2A})(R^{3A})C(═O)N(R)—
wherein:
X_{A} is a bond, —O—, —N(R)—, —CR^{4A}R^{5A}—, —O—CR^{4A}R^{5A}, —N(R)—CR^{4A}R^{5A}—, —O—CR^{4A}R^{5A}O—, —N(R)—CR^{4A}R^{5A}—O, —N(R)—CR^{4A}R^{5A}—N(R)—, —O—CR^{4A}R^{5A}—N(R)—, —CR^{4A}R^{5A}—O—, —CR^{4A}R^{5A}—N(R)—, —O—CR^{4A}R^{5A}—CR^{6A}R^{7A}—, —N(R)—CR^{4A}R^{5A}—CR^{6A}R^{7A}—, —CR^{6A}R^{7A}—CR^{4A}R^{5A}—O—, —CR^{6A}R^{7A}—CR^{4A}R^{5A}—N(R)—, or —CR^{6A}R^{7A}—CR^{4A}R^{5A}—;
R is independently hydrogen or optionally substituted C₁₋₆ aliphatic;
R^{4A} and R^{5A} are independently selected from the group consisting of hydrogen, halo, —CN, —NO₂, optionally substituted aliphatic, optionally substituted carbocyclyl, optionally substituted phenyl, optionally substituted heterocyclyl, optionally substituted heteroaryl, —OR^{A}, —N(R^{B})₂, —SR^{A}, —C(═O)R^{A}, —C(O)OR^{A}, —C(O)SR^{A}, —C(O)N(R^{B})₂, —C(O)N(R^{B})N(R^{B})₂, —OC(O)R^{A}, —OC(O)N(R^{B})₂, —NR^{B}C(O)R^{A}, —NR^{B}C(O)N(R^{B})₂, —NR^{B}C(O)N(R^{B})N(R^{B})₂, —NR^{B}C(O)OR^{A}, —SC(O)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(O)R^{A}, —OS(O)₂R^{A}, —SO₂R^{A}, —NR^{B}SO₂R^{A}, or —SO₂N(R^{B})₂; or R^{4A} and R^{5A} are taken together with their intervening atoms to form an optionally substituted carbocyclic or heterocyclic ring;
R^{6A} and R^{7A} are independently selected from the group consisting of hydrogen, halo, —CN, —NO₂, optionally substituted aliphatic, optionally substituted carbocyclyl, optionally substituted phenyl, optionally substituted heterocyclyl, optionally substituted heteroaryl, —OR^{A}, —N(R^{B})₂, —SR^{A}, —C(═O)R^{A}, —C(O)OR^{A}, —C(O)SR^{A}, —C(O)N(R^{B})₂, —C(O)N(R^{B})N(R^{B})₂, —OC(O)R^{A}, —OC(O)N(R^{B})₂, —NR^{B}C(O)R^{A}, —NR^{B}C(O)N(R^{B})₂, —NR^{B}C(O)N(R^{B})N(R^{B})₂, —NR^{B}C(O)OR^{A}, —SC(O)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(O)R^{A}, —OS(O)₂R^{A}, —SO₂R^{A}, —NR^{B}SO₂R^{A}, or —SO₂N(R^{B})₂; or R^{6A} and R^{7A} are taken together with their intervening atoms to form an optionally substituted carbocyclic or heterocyclic ring;
R^{2A} and R^{3A} are independently selected from the group consisting of hydrogen, halo, —CN, —NO₂, optionally substituted aliphatic, optionally substituted carbocyclyl, optionally substituted phenyl, optionally substituted heterocyclyl, optionally substituted heteroaryl, —OR^{A}, —N(R^{B})₂, —SR^{A}, —C(═O)R^{A}, —C(O)OR^{A}, —C(O)SR^{A}, —C(O)N(R^{B})₂, —C(O)N(R^{B})N(R^{B})₂, —OC(O)R^{A}, —OC(O)N(R^{B})₂, —NR^{B}C(O)R^{A}, —NR^{B}C(O)N(R^{B})₂, —NR^{B}C(O)N(R^{B})N(R^{B})₂, —NR^{B}C(O)OR^{A}, —SC(O)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(O)R^{A}, —OS(O)₂R^{A}, —SO₂R^{A}, —NR^{B}SO₂R^{A}, or —SO₂N(R^{B})₂; or R^{2A} and R^{3A} are taken together with their intervening atoms to form an optionally substituted carbocyclic or heterocyclic ring;
each R^{A} is independently selected from the group consisting of hydrogen, optionally substituted aliphatic, optionally substituted carbocyclyl, optionally substituted heterocyclyl, optionally substituted aryl, and optionally substituted heteroaryl; and
each R^{B} is independently selected from the group consisting of hydrogen, optionally substituted aliphatic, optionally substituted carbocyclyl, optionally substituted heterocyclyl, optionally substituted aryl, and optionally substituted heteroaryl, or two R^{B} groups are taken together with their intervening atoms to form an optionally substituted heterocyclic ring.

20. The compound of any one of claims 1-18, wherein L_{z} is a linker L_{B}, wherein L_{B} is —N(R)C(O)—, —C(O)N(R)—, —N(R)C(O)N(R)—, —N(R)C(O)O—, or —OC(O)N(R)—, and each R is independently hydrogen or optionally substituted C₁₋₆ aliphatic.

21. The compound of any one of claims 1-18, wherein L_{z} is a linker L_{D}, wherein:
L_{D} is the linker L_{B} wherein L_{B} is —N(R)C(O)—, —C(O)N(R)—, —N(R)C(O)N(R)—, —N(R)C(O)O—, or —OC(O)N(R)— and each R is independently hydrogen or optionally substituted C₁₋₆ aliphatic; or
L_{D} is a linker selected from the group consisting of —O—, —N(R)—, —C(R^{2A})(R^{3A})—, —O—CR^{2A}R^{3A}, —N(R)—CR^{2A}R^{3A}—, —O—CR^{2A}R^{3A}—O—, —N(R)—CR^{2A}R^{3A}—O, —N(R)—CR^{2A}R^{3A}—N(R)—, —O—CR^{2A}R^{3A}—N(R)_, —CR^{2A}R^{3A}—O—, —CR^{2A}R^{3A}—N(R), —O—CR^{2A}R^{3A}—CR⁹R¹⁰—, —N(R)—CR^{2A}R^{3A}CR⁹R¹⁰—, —CR^{2A}R^{3A}—CR⁹R¹⁰—O—, —CR^{2A}R^{3A}—CR⁹R—N(R)—, or —CR^{2A}R^{3A}—CR⁹R¹⁰—;
each R is independently hydrogen or optionally substituted C₁₋₆ aliphatic;
R^{2A} and R^{3A} are independently selected from the group consisting of hydrogen, halo, —CN, —NO₂, optionally substituted aliphatic, optionally substituted carbocyclyl; optionally substituted phenyl, optionally substituted heterocyclyl, optionally substituted heteroaryl, —OR^{A}, —N(R^{B})₂, —SR^{A}, —C(═O)R^{A}, —C(O)OR^{A}, —C(O)SR^{A}, —C(O)N(R^{B})₂, —C(O)N(R^{B})N(R^{B})₂, —OC(O)R^{A}, —OC(O)N(R^{B})₂, —NR^{B}C(O)R^{A}, —NR^{B}C(O)N(R^{B})₂, —NR^{B}C(O)N(R^{B})N(R^{B})₂, —NR^{B}C(O)OR^{A}, —SC(O)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(O)R^{A}, —OS(O)₂R^{A}, —SO₂R^{A}, —NR^{B}SO₂R^{A}, or —SO₂N(R^{B})₂; or R^{2A} and R^{3A} are taken together with their intervening atoms to form an optionally substituted carbocyclic or heterocyclic ring;
each R^{A} is independently selected from the group consisting of hydrogen, optionally substituted aliphatic, optionally substituted carbocyclyl, optionally substituted heterocyclyl, optionally substituted aryl, and optionally substituted heteroaryl;
each R^{B} is independently selected from the group consisting of hydrogen, optionally substituted aliphatic, optionally substituted carbocyclyl, optionally substituted heterocyclyl, optionally substituted aryl, and optionally substituted heteroaryl, or two R^{B} groups are taken together with their intervening atoms to form an optionally substituted heterocyclic ring; and
R⁹ and R¹⁰ are independently selected from the group consisting of hydrogen, halo, —CN, —NO₂, optionally substituted aliphatic, optionally substituted carbocyclyl; optionally substituted phenyl, optionally substituted heterocyclyl, optionally substituted heteroaryl, —OR^{A}, —N(R^{B})₂, —SR^{A}, —C(═O)R^{A}, —C(O)OR^{A}, —C(O)SR^{A}, —C(O)N(R^{B})₂, —C(O)N(R^{B})N(R^{B})₂, —OC(O)R^{A}, —OC(O)N(R^{B})₂, —NR^{B}C(O)R^{A}, —NR^{B}C(O)N(R^{B})₂, —NR^{B}C(O)N(R^{B})N(R^{B})₂, —NR^{B}C(O)OR^{A}, —SC(O)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(O)R^{A}, —OS(O)₂R^{A}, —SO₂R^{A}, —NR^{B}SO₂R^{A}, or —SO₂N(R^{B})₂; or R⁹ and R¹⁰ are taken together with their intervening atoms to form an optionally substituted carbocyclic or heterocyclic ring.

22. The compound of any one of claims 1-18, wherein L_{z} is absent.

23. The compound of any one of claims 1-22, wherein Ring Z is a group Cy^{A}, wherein:
Cy^{A} is a monocyclic or bicyclic, saturated, partially unsaturated, or aromatic ring having 0-4 heteroatoms independently selected from nitrogen, oxygen, and sulfur, wherein Cy^{A} is substituted with 0, 1, 2, 3, or 4 R^{y} groups; and
each R^{y} is independently selected from the group consisting of halo, —CN, —NO₂, optionally substituted aliphatic, optionally substituted carbocyclyl, optionally substituted aryl, optionally substituted heterocyclyl, optionally substituted heteroaryl, —OR^{A}, —N(R^{B})₂, —SR^{A}, —C(═O)R^{A}, —C(O)OR^{A}, —C(O)SR^{A}, —C(O)N(R^{B})₂, —C(O)N(R^{B})N(R^{B})₂, —OC(O)R^{A}, —OC(O)N(R^{B})₂, —NR^{B}C(O)R^{A}, —NR^{B}C(O)N(R^{B})₂, —NR^{B}C(O)N(R^{B})N(R^{B})₂, —NR^{B}C(O)OR^{A}, —SC(O)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(O)R^{A}, —OS(O)₂R^{A}, —SO₂R^{A}, —NR^{B}SO₂R^{A}, or —SO₂N(R^{B})₂.

24. The compound of claim 23, wherein the compound is of Formula (A-I^{A}): 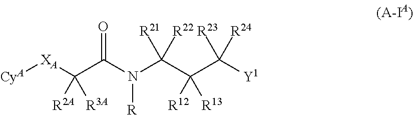
or a pharmaceutically acceptable salt thereof.

25. The compound of claim 24, wherein the compound is a compound of formula: 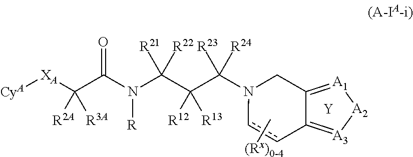
or a pharmaceutically acceptable salt thereof, wherein each instance of A₁ and A₃ is independently N, CH, or CR^{x}, and A₂ is O, S, NH, or NR^{x}.

26. The compound of claim 24, wherein the compound is a compound of formula: 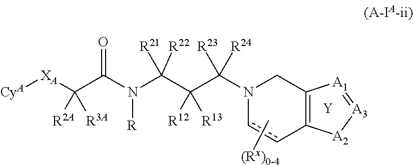
or a pharmaceutically acceptable salt thereof, wherein each instance of A₁ and A₃ is independently N, CH, or CR^{x}, and A₂ is O, S, NH, or NR^{x}.

27. The compound of claim 24, wherein the compound is a compound of formula: 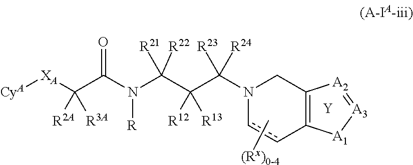
or a pharmaceutically acceptable salt thereof, wherein each instance of A₁ and A₃ is independently N, CH, or CR^{x}, and A₂ is O, S, NH, or NR^{x}.

28. The compound of claim 24, wherein the compound is a compound of formula: 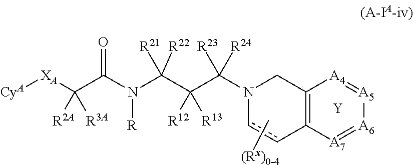
or a pharmaceutically acceptable salt thereof, wherein each instance of A₄, A₅, A₆, and A₇ is independently N, CH, or CR^{x}, provided at least one of A₄, A₅, A₆, and A₇ is N.

29. The compound of any one of claims 1-18, wherein Ring Z is a group Ar, wherein:
Ar is a monocyclic or bicyclic aromatic ring having 0-4 heteroatoms independently selected from nitrogen, oxygen, and sulfur, wherein Ar is substituted with 0, 1, 2, 3, 4, or 5 R^{y} groups, as valency permits;
each R^{y} is independently selected from the group consisting of halo, —CN, —NO₂, optionally substituted aliphatic, optionally substituted carbocyclyl, optionally substituted aryl, optionally substituted heterocyclyl, optionally substituted heteroaryl, —OR^{A}, —N(R^{B})₂, —SR^{A}, —C(═O)R^{A}, —C(O)OR^{A}, —C(O)SR^{A}, —C(O)N(R^{B})₂, —C(O)N(R^{B})N(R^{B})₂, —OC(O)R^{A}, —OC(O)N(R^{B})₂, —NR^{B}C(O)R^{A}, —NR^{B}C(O)N(R^{B})₂, —NR^{B}C(O)N(R^{B})N(R^{B})₂, —NR^{B}C(O)OR^{A}, —SC(O)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(O)R^{A}, —OS(O)₂R^{A}, —SO₂R^{A}, —NR^{B}SO₂R^{A}, or —SO₂N(R^{B})₂;
each R^{A} is independently selected from the group consisting of hydrogen, optionally substituted aliphatic, optionally substituted carbocyclyl, optionally substituted heterocyclyl, optionally substituted aryl, and optionally substituted heteroaryl; and
each R^{B} is independently selected from the group consisting of hydrogen, optionally substituted aliphatic, optionally substituted carbocyclyl, optionally substituted heterocyclyl, optionally substituted aryl, and optionally substituted heteroaryl, or two R^{B} groups are taken together with their intervening atoms to form an optionally substituted heterocyclic ring.

30. The compound of claim 29, wherein the compound is of Formula (A-I^{B}): 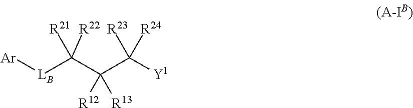
or a pharmaceutically acceptable salt thereof.

31. The compound of any one of claims 1-22, wherein Ring Z is Ring C of formula: 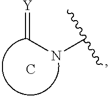
wherein:
Ring C is an optionally substituted, 5- to 12-membered, monocyclic or bicyclic, heterocyclyl or heteroaryl having 1-4 heteroatoms independently selected from nitrogen, oxygen, and sulfur; and
Y is O or S.

32. The compound of claim 31, wherein the compound is of Formula (A-I^{C}): 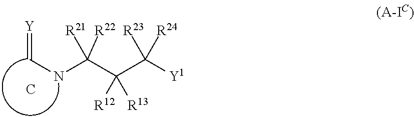
or a pharmaceutically acceptable salt thereof.

33. The compound of any one of claims 1-22, wherein Ring Z is Ring A of formula: 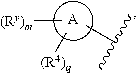
wherein:
Ring A is a monocyclic or bicyclic, saturated, partially unsaturated, or aromatic ring having 0-4 heteroatoms independently selected from nitrogen, oxygen, and sulfur;
R⁴ is -L₁-Cy^{D};
L₁ is a bond, —O—, —S—, —N(R)—, —C(O)—, —C(O)N(R)—, —N(R)C(O)N(R)—, —N(R)C(O)—, —N(R)C(O)O—, —OC(O)N(R)—, —SO₂—, —SO₂N(R)—, —N(R)SO₂—, —OC(O)—, —C(O)O—, or an optionally substituted, straight or branched, C₁₋₆ aliphatic chain wherein one, two, or three methylene units of L₁ are optionally and independently replaced by —O—, —S—, —N(R)—, —C(O)—, —C(O)N(R)—, —N(R)C(O)N(R)—, —N(R)C(O)—, —N(R)C(O)O—, —OC(O)N(R)—, —SO₂—, —SO₂N(R)—, —N(R)SO₂—, —OC(O)—, or —C(O)O—;
each R is independently hydrogen or optionally substituted C₁₋₆ aliphatic;
Cy^{D} is an optionally substituted, monocyclic, bicyclic or tricyclic, saturated, partially unsaturated, or aromatic ring having 0-4 heteroatoms independently selected from nitrogen, oxygen, and sulfur;
each R^{y} is independently selected from the group consisting of halo, —CN, —NO₂, optionally substituted aliphatic, optionally substituted carbocyclyl; optionally substituted aryl, optionally substituted heterocyclyl, optionally substituted heteroaryl, —OR^{A}, —N(R^{B})₂, —SR^{A}, —C(═O)R^{A}, —C(O)OR^{A}, —C(O)SR^{A}, —C(O)N(R^{B})₂, —C(O)N(R^{B})N(R^{B})₂, —OC(O)R^{A}, —OC(O)N(R^{B})₂, —NR^{B}C(O)R^{A}, —NR^{B}C(O)N(R^{B})₂, —NR^{B}C(O)N(R^{B})N(R^{B})₂, —NR^{B}C(O)OR^{A}, —SC(O)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(O)R^{A}, —OS(O)₂R^{A}, —SO₂R^{A}, —NR^{B}SO₂R^{A}, or —SO₂N(R^{B})₂;
m is 0, 1, 2, 3, 4, 5, 6, 7, or 8, as valency permits; and
q is 0 or 1.

34. The compound of claim 33, wherein the compound is of Formula (A-I^{D}): 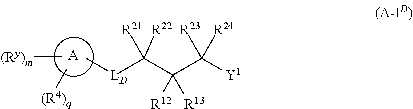
or a pharmaceutically acceptable salt thereof.

35. The compound of claim 34, wherein the compound is of Formula (A-V^{D}): 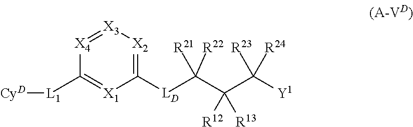
or a pharmaceutically acceptable salt thereof, wherein X₁, X₂, X₃, and X₄ are independently selected from the group consisting of N, CH, and CR^{y}, provided that at least one of X₂, X₃, and X₄ is not N

36. The compound of claim 35, wherein the compound is a compound of formula: 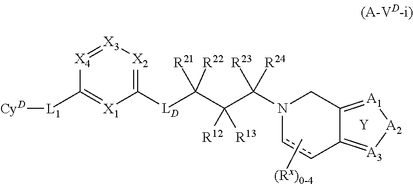
or a pharmaceutically acceptable salt thereof, wherein each instance of A₁ and A₃ is independently N, CH, or CR^{x}, and A₂ is O, S, NH, or NR^{x}.

37. The compound of claim 35, wherein the compound is a compound of formula: 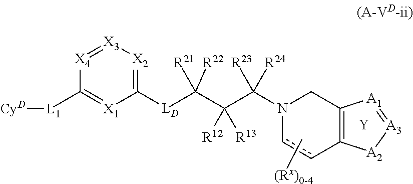
or a pharmaceutically acceptable salt thereof, wherein each instance of A₁ and A₃ is independently N, CH, or CR^{x}, and A₂ is O, S, NH, or NR^{x}.

38. The compound of claim 35, wherein the compound is a compound of formula: 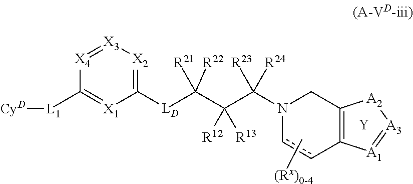
or a pharmaceutically acceptable salt thereof, wherein each instance of A₁ and A₃ is independently N, CH, or CR^{x}, and A₂ is O, S, NH, or NR^{x}.

39. The compound of claim 35, wherein the compound is a compound of formula: 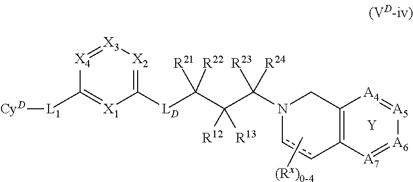
or a pharmaceutically acceptable salt thereof, wherein each instance of A₄, A₅, A₆, and A₇ is independently N, CH, or CR^{x}, provided at least one of A₄, A₅, A₆, and A₇ is N.

40. The compound of claim 35, wherein the compound is of Formula: 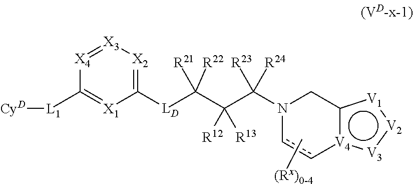
or a pharmaceutically acceptable salt thereof,
wherein:
each instance of V₁, V₂, and V₃ is independently O, S, N, NH, NR^{x}, CH, or CR^{x}; and
V₄ is N or C.

41. The compound of claim 35, wherein the compound is of Formula: 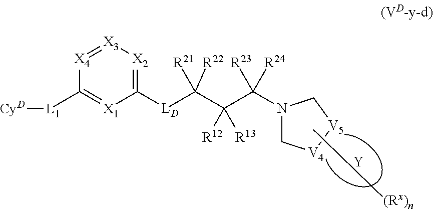
or a pharmaceutically acceptable salt thereof.

42. The compound of any one of claims 1-15 and 19-41, wherein R¹² is hydrogen and R¹³ is —OR¹.

43. The compound of any one of claims 1-15 and 19-41, wherein R¹² is optionally substituted C₁₋₃alkyl and R¹³ is —OR¹.

44. The compound of any one of claims 1-15 and 19-41, wherein R¹² is hydrogen and R¹³ is hydrogen.

45. The compound of any one of claims 1-15 and 19-41, wherein R¹² is hydrogen and R¹³ is halogen.

46. The compound of any one of claims 1-15 and 19-41, wherein the carbon attached to R¹² has (S)-stereochemistry.

47. The compound of any one of claims 1-15 and 19-41, wherein the carbon attached to R¹² has (R)-stereochemistry.

48. The compound of claims 1-47, wherein the compound is selected from the group consisting of the compounds in Table 1A, 1B, 1C, 1D, and 1E and pharmaceutically acceptable salts thereof.

49. A pharmaceutical composition comprising a compound of any one of the preceding claims or a pharmaceutically acceptable salt thereof, and a pharmaceutically acceptable excipient.

50. A kit or packaged pharmaceutical comprising a compound of any one of the preceding claims or a pharmaceutically acceptable salt thereof, and instructions for use thereof.

51. A method of inhibiting PRMT5 comprising contacting a cell with an effective amount of a compound of any one of the preceding claims or a pharmaceutically acceptable salt thereof.

52. A method of altering gene expression comprising contacting a cell with an effective amount of a compound of any one of the preceding claims or a pharmaceutically acceptable salt thereof.

53. A method of altering transcription comprising contacting a cell with an effective amount of a compound of any one of the preceding claims or a pharmaceutically acceptable salt thereof.

54. The method of any one of claims 51-53, wherein the cell is in vitro.

55. The method of any one of claims 51-53, wherein the cell is in a subject.

56. A method of treating a PRMT5-mediated disorder, comprising administering to a subject in need thereof a therapeutically effective amount of a compound of any one of the preceding claims, or a pharmaceutically acceptable salt thereof, or a pharmaceutical composition of claim 49.

57. The method of claim 56, wherein the disorder is a proliferative disorder.

58. The method of claim 57, wherein the proliferative disorder is cancer.

59. The method of claim 58, wherein the cancer is hematopoietic cancer, lung cancer, prostate cancer, melanoma, or pancreatic cancer.

60. The method of claim 56, wherein the disorder is a metabolic disorder.

61. The method of claim 60, wherein the metabolic disorder is diabetes.

62. The method of claim 60, wherein the metabolic disorder is obesity.

63. The method of claim 56, wherein the disorder is a blood disorder.

64. The method of claim 63, wherein the blood disorder is a hemoglobinopathy.

65. The method of claim 63, wherein the blood disorder is sickle cell anemia.

66. The method of claim 63, wherein the blood disorder is β-thalessemia.
